SEQUENCE LISTING (175 sequences)

SEQ ID NO: 1 - DNA, 141 bp, Saccharopolyspora spinosa
gccgcaccaagcgagcaatgccgccccggcggtcccgaccgcgggaccccggggcggtcgcacgtccggggcagcgggacttgtcgatggaacaggtacggcctcaatagatcaggtaccgatgaagggctgttggaatca

SEQ ID NO: 2 - DNA, 198 bp, Saccharopolyspora spinosa
ggaccgagcgggaggcaacgcctcgcgaaggcgaccggggagcaatcccctccagttcggcggcggacgggccgccaccccgcaaggacagtgttcttccgggatcggcggcccgctcgtcacctacccgacaggactccgcctggcacaacaagtcgtacggcggaaagttaacaagtccaggaggacaatccagtg

SEQ ID NO: 3 - DNA, 232 bp, Saccharopolyspora spinosa
gggactgtttgaaagtggctagcgtagcggtgcgggtagcggaacctcagaggccttctcgctctgggatccccgacatcatgaatgccaattcaccaggtcggggctgtcctcgcgagaagacccctgagaacccgcggcggtgcgagttgagtcccacaccgcaagcggctaccgccgcttataagacaggctctaaccgagtgaaaggcgctgagagttgagcaccctc

SEQ ID NO: 4 - DNA, 361 bp, Saccharopolyspora spinosa
tagaaactgttcatcgactggctccgcgtggcggtgcggatagcggaacctaaaattctcgctgtgggatccccgacatcatgaatgccattcacctagtcgggggtgtcctcgcgagatggctcggcgtgtggggtgtcctcgcaggacactgttgagaacccgcggcggcggtgcgagttgcgcgcgtgggctaagcggcttcgccgcttgaaagacaaagacgtagcgggagtgagtgccagggcgggccgttgtccgctttggcactcgcgtccgtttcgggggccggtggtcggcggactgcggggttctggttcgatcttgggtcgtagctccgggtaattcccggggatctacc

SEQ ID NO: 5 - DNA, 303 bp, Saccharopolyspora spinosa
tcgcccacaggacaggaacacagcgtgtcgatgaaacgtcatactggtgttggacgaaaacccagatggagcagtaccgagcaaagtcgacttcgagtggggcatttcgagtgcggtcgatgatcattgacgcgagtggaccggggctcattccccgcaagctggtctttcctgatcgattttgtgaccgacctcgtcgaacgaacggccgtactgtgtgtcaacctcgcgaatcgggccgctagcctggtacctgagtgtgtctgtaaatcacgagcatatggatcgaggcgaacgccagcc

SEQ ID NO: 6 - DNA, 253 bp, Saccharopolyspora spinosa
ctggcgaggtgccgaccataactcgatctacacgagcgtggacttgcaacgttgaccgtttacatccgtgtagatatcctcgtcggagtccggccagggagatggcgtctgcacgtcgacgatgctgcccggcggaccgctttgccagccgaaggatgagcgccgtgacggtgtcgtcaaccaaaccggcccgctgtgcgacgaggcggcttcccgcccgcggcacggggtcacgcacgaaaggagtgcggcg

SEQ ID NO: 7 - DNA, 168 bp, Saccharopolyspora spinosa
tatccacgcgctgtggttctcgtgggcaccctctgcccacagcaaaagaagatctttcgtcgctgcgcagttctagcggaaggatattgtgtagatgcggatttctgatgtaatttttcttgacaagtgatgcggggcaccgcaccacacggcgccgggaggccgaac

SEQ ID NO: 8 - DNA, 363 bp, Saccharopolyspora spinosa
ctgctcgccatactcaacgacattcttccgtcactggaaacgacgaaggcgatgtcgctgcccgaaagtgtggctacaatacgggacctcggcatcgtaatgggctccgtcaagaggcacggcgttgaaccagtggtcgcgattcccgaactggaagcgccgttgcgtgccataggcgaacgcaccggcatgatcccacgggacaccatccatcactacatacggtggaatccgacggggcggcgtgagcgcatgtacacgggcgagccgatggagaagttgctcatggcctctgtgcgcatcagtttgcccaggctgagcgctgctgtcgatgtgtgcaccgctctgcacaccgccgaggcc

SEQ ID NO: 9 - DNA, 300 bp, Saccharopolyspora spinosa
gcctttttgccgaaatcgcgcggccagttcgtccaatgcgcaagtcgcgatctccggcggcgcggtgcgaagcggccgcgcggtgcaggtgaccccacagtctttccggggcggttcttggcggggttcggcgcgaagggattcaagatcgttcttctgaaacgtccaagacatcgatctttgtgccgcttttaacgtgttcgactgcgttgccgccctccgcctcttggcggaactggcagtcttaaggtggaacctgttggcacaatgaggtgccgtcaagcgtggagctgcctgaag

SEQ ID NO: 10 - DNA, 322 bp, Saccharopolyspora spinosa
gcgaacgcaagccatttcggcgcgggccaggccgtgtcgggtcgggctgggtgcgggcgggcttacctgcgcaactctggctgtgcaagggatcgcttatccgccatgccatcaggttgaccagctctccgagtgcagtcgtcggctgccggcagggcgttgacatcgggcgctttgacgccagcggggtgaggtgatgtgtacaagcgccgttgccgacgggtggatcttgctcgcggcttcggcgcagacaggttcggcgaaaactactcttgcgtgctcggataatccgtgctcggattacatgcggaggtggtcaacg

SEQ ID NO: 11 - DNA, 149 bp, Saccharopolyspora spinosa
ctcaggatctggacactaaattccatcttttgggtgaaagttgactggaacgatttagaaggtgacggctttgtgacggggcattgctgtgaaatggttctcacttatgtttacgctcgtctgacgcggcggtgaatgatccgccgcgc

SEQ ID NO: 12 - DNA, 199 bp, Saccharopolyspora spinosa
acgacggagcgaccctaacatcgacacaccggtcgcctcccgtgacagcacgaccgaagaatctaaagctgccctttttaactagagaattctgaacaaaaaggcaagatgtcaccctggtcacaatccggccttccgcgcgcggcattgacgcggtaaagtcccgggtcgccatcgacacgaggcagggtgccctggc

SEQ ID NO: 13 - DNA, 41 bp, Artificial Sequence
  Synthetic promoter P21
tgtgcgggctctaacacgtcctagtatggtaggatgagcaa

SEQ ID NO: 14 - DNA, 41 bp, Artificial Sequence
  Synthetic promoter PA9
ccgggcggcttcctcatgcttgacttgactaggataaaggg

SEQ ID NO: 15 - DNA, 41 bp, Artificial Sequence
  Synthetic promoter PA3
tagcagggctccaaaactaacgcctgatgtaggatcagatg

SEQ ID NO: 16 - DNA, 41 bp, Artificial Sequence
  Synthetic promoter PB4
gctgtaggctgttaatatatttcggtgtgtaggatacgggc

SEQ ID NO: 17 - DNA, 41 bp, Artificial Sequence
  Synthetic promoter PB12
cgggatggcttatgaaggattgtctcacttaggatagagca

SEQ ID NO: 18 - DNA, 41 bp, Artificial Sequence
  Synthetic promoter PB1
cgtcagggctactctggcaaccaagcgattaggattgaagg

SEQ ID NO: 19 - DNA, 41 bp, Artificial Sequence
  Synthetic promoter PC1
actttcggctaaaaagcaattcattcaattaggatggaaga

SEQ ID NO: 20 - DNA, 41 bp, Artificial Sequence
  Synthetic promoter P72
ctaattggctacgtcatagagagattctttaggatgagaaa

SEQ ID NO: 21 - DNA, 44 bp, Artificial Sequence
  Synthetic promoter P-C4-1
ggcaactaggttgacgtatttttccgttaggcctagggtgagtg

SEQ ID NO: 22 - DNA, 44 bp, Artificial Sequence
  Synthetic promoter P-A5-19
tatgcgttgcttgaccaaacctatgtatagggatagggttggtc

SEQ ID NO: 23 - DNA, 44 bp, Artificial Sequence
  Synthetic promoter P-C4-14
ccctcgctggttgacacagttagtcagattgcctacgatttcgt

SEQ ID NO: 24 - DNA, 44 bp, Artificial Sequence
  Synthetic promoter P-D1-7
aattgcccacttgacgttgagagtgaagcaatataggttaacct

SEQ ID NO: 25 - DNA, 242 bp, Saccharopolyspora spinosa
aaccactgcgacgagcggtatttgggggaagtaaagaggtgacccacgactcactgtcggtgatcgatatcgacccggaaacgacttgataacgacgctctgatcagcacaaataccccggatcgaagcacccacccccactgttactgtgatcagcgtcacatgatctcaggtttccgatctccgtgttggttacgtagtgtcgctcctcggtcggccccgaaccgatcagcaaggagcgg

SEQ ID NO: 26 - DNA, 65 bp, Saccharopolyspora spinosa
cagcaaacctgtggaccatcaccaacaccgaaacgtctaatgggcaagtcaaccttcgcggcgaa

SEQ ID NO: 27 - DNA, 201 bp, Saccharopolyspora spinosa
tccggtggcctggtcaaacgccgccgggcacggcgtctggtatctctgaatgtgtgacgatgacggccccctccccccactcgctgttcaacgatcttcccttgcccggtacggtgagctcggcgtggcgtgcgaggcacgcgatttccgccggacaaatccgaatcgcttgaacgcgtaacaccagggctgctgtctgcg

SEQ ID NO: 28 - DNA, 300 bp, Saccharopolyspora spinosa
gcggcaaaccgtcgcagacaccccgaaacgtcgtgatcgtcctgccctactgcccttgtgaagatcgtctcggatcttcgctcgtggtcccacctccattccggtggcctggtcaaacgccgccgggcacggcgtctggtatctctgaatgtgtgacgatgacggccccctccccccactcgctgttcaacgatcttcccttgcccggtacggtgagctcggcgtggcgtgcgaggcacgcgatttccgccggacaaatccgaatcgcttgaacgcgtaacaccagggctgctgtctgcg

SEQ ID NO: 29 - DNA, 220 bp, Saccharopolyspora spinosa
ctgaatgcagccgtaagttattggatcacctaggaatcgggtcacttttcccctgccggaatgtgtgcctgcttacttagcgtgccttgttcacctctcgttcacttcgatggcggcgatcgtccactccgactccttagcgtccgtgtcgagcggccaaagcacgagcctgcgcgaggctcggccgcgcaaccgcagggtttccaactggaggaacgaa

SEQ ID NO: 30 - DNA, 177 bp, Saccharopolyspora spinosa
acttttcccctgccggaatgtgtgcctgcttacttagcgtgccttgttcacctctcgttcacttcgatggcggcgatcgtccactccgactccttagcgtccgtgtcgagcggccaaagcacgagcctgcgcgaggctcggccgcgcaaccgcagggtttccaactggaggaacgaa

SEQ ID NO: 31 - DNA, 200 bp, Saccharopolyspora spinosa
gtgattccggctagactgctactttgcgctgccctctttccgtctgtcctgcaccggaccgtaggatggtgggcgccattgcacccttgacagctgtgttagcggagtgtgacagcggatacggaccccgtcggtcgcattcgccgggcacctttcgccgacgcggctgtagccagttcagagtcccggaaggacgcatc

SEQ ID NO: 32 - DNA, 299 bp, Saccharopolyspora spinosa
tgatcgaagcgtgatctcttgactggcggcgcgcgcgggttcactctagtcctcaacgcggggctgggctgccgtcggtgtgccccctcgacagctggcgtgattccggctagactgctactttgcgctgccctctttccgtctgtcctgcaccggaccgtaggatggtgggcgccattgcacccttgacagctgtgttagcggagtgtgacagcggatacggaccccgtcggtcgcattcgccgggcacctttcgccgacgcggctgtagccagttcagagtcccggaaggacgcatc

SEQ ID NO: 33 - DNA, 240 bp, Saccharopolyspora spinosa
cgctccgccgcatcggttacggcgcttgcactcgactggcgagagtgctaaacacggtattggcactcagcaaggttgagtgccaggtcgggacggtgaggccgtctccggcggtgccaccagacggcgccgccgcacggtcgtccgtcgcgggcaccgagcctggccgagcacgagtcctgccgtggggtgcgcaaacccaccaccgcggcgtccagacaggtggaggaccacaccgca

SEQ ID NO: 34 - DNA, 242 bp, Saccharopolyspora spinosa
accggatcagcagttccacgccgatctaataaggaccaactcggctcggcggaagtccggtaggagcgaagttactgtcctcagaggtctgaggtccagtggaagaggcgacgaaacaaggagattcgtctctcaccgtaaagagtgaaaaaatctagcgaggcggctgacggctttcggttcgacttgcgagtcggctaggttcgtgatcacgaactccgattgaaggtcctaacaggagt

SEQ ID NO: 35 - DNA, 300 bp, Saccharopolyspora spinosa
ctccagccacagccggagcatctccctgatctcgatcaccgaaacctcccggtagctcattccgccaatacgagcggccgagcgaccggaaaccccgcagccacgccgggcggtcccataactggcaaaccaccagcccagacggtcccatcagtggcaaacaggtggtcccatgctcctggcaaaaccggctctgaggtggtcccttactcctggcagccgacaggcgtcgttgcgcagtggcggacagggcacggatgactcctatgaggtagtcgattacgtgctaccgtctacgcc

SEQ ID NO: 36 - DNA, 359 bp, Saccharopolyspora spinosa
ggcggactaagacttaggtctacctaagcgcggtgcaacttaacccagcgttccgcgcggcacaagcaccttgttcgaaaccgggttccaggtcacctggaaacccatttgagaccggatcgacgcactttcccacgcctatacgggtatctggcgaatggcggaatctgactttgggttcggcagtgacctggacttatatgtcgatgtgcgcatcagtcgacgtgatactcgcgactaaccgcaggtgatttgccgaacgggtctgcgtatttccctgcggcgagttagggtgcccttgcttgccttgaacattgctctacctcatcaggactccttcgaagggaagtgagctgctc

SEQ ID NO: 37 - DNA, 433 bp, Saccharopolyspora spinosa
aaccactgcgacgagcggtatttgggggaagtaaagaggtgacccacgactcactgtcggtgatcgatatcgacccggaaacgacttgataacgacgctctgatcagcacaaataccccggatcgaagcacccacccccactgttactgtgatcagcgtcacatgatctcaggtttccgatctccgtgttggttacgtagtgtcgctcctcggtcggccccgaaccgatcagcaaggagcggaagcccgcagcgccgaaccctgtccagcaggcttccagaccccgaaacgaagaacaccggacagggacgggggacccaacacccgggctccccgaagccctaggggtgaagccggctcccccgagccggccgggctgcctctcagcccgaacccgacagctcacctcgcaggcgcggcaggagagaggaac

SEQ ID NO: 38 - DNA, 115 bp, Saccharopolyspora spinosa
gggcgcgccgcaattcgatgacgttcatgcgccgtgtcggggaatcgccggtggcggcgccagcagagactgaacttactggtggtgtgtccaggaatcggaggggcagtaccga

SEQ ID NO: 39 - DNA, 496 bp, Saccharopolyspora spinosa
acttcggaccccagtctctttcccccgattagcgcagcagcccctactcccattggccaggatttggaaaatgcgctgcgtatgtcgatcgccgttgacgtccaacggacttccggcggcaacaatagtgtgtcacggcaggaatgtcacgcgaccatcgaagatctttgggtcgccgcacctggtttcacgcgaacgagtgaaatgcgcgagctccgctcgatcggggtgggccggacctgtacggtgatcaccgttggttctgcggggattcatggggaagatttgcgctggctgtttgcctcctggccggatagttatagtcggtaccgccgcatgcggcggtaaccgcgaattaactgacggctagtttgccgtcttttctctctgtgtgtttcctgctcggttccagaaaattacgagaaggtgaacgttgcagagatcaggcataccggtgttgccaggtggcgcaccaacatcgcagcaggttgggcag

SEQ ID NO: 40 - DNA, 202 bp, Saccharopolyspora spinosa
cactcgttcgcgtgaaaccaggtgcggcgacccaaagatcttcgatggtcgcgtgacattcctgccgtgacacactattgttgccgccggaagtccgttggacgtcaacggcgatcgacatacgcagcgcattttccaaatcctggccaatgggagtaggggctgctgcgctaatcgggggaaagagactggggtccgaagt

SEQ ID NO: 41 - DNA, 379 bp, Saccharopolyspora spinosa
gaactctccgatcgcaattgaacacccgggaagcatgccaagaatcacagaaatctctgatatcccccgggaaacgccgctttcgcaagccaaatcttaggccttccaggtgatggtagcgatcttgacaagcgcgagcaggtcgttcccgctagcctgggctctaccgagtcgggtgtgccgggtagatcgaggatttctgagtcaatgagcgcttctccttgctccgctgtcctgatgtcccgcaccgcatcgaaccagggcaggaaggtgtaaggcgccgagacagcacactgtcccgctgggacgtcataacgcgattcgccacgggcatcgctcatctcctgaaggcaaggcgcgaagactgatcgtcgcctgc

SEQ ID NO: 42 - DNA, 261 bp, Saccharopolyspora spinosa
gcgatcttgacaagcgcgagcaggtcgttcccgctagcctgggctctaccgagtcgggtgtgccgggtagatcgaggatttctgagtcaatgagcgcttctccttgctccgctgtcctgatgtcccgcaccgcatcgaaccagggcaggaaggtgtaaggcgccgagacagcacactgtcccgctgggacgtcataacgcgattcgccacgggcatcgctcatctcctgaaggcaaggcgcgaagactgatcgtcgcctgc

SEQ ID NO: 43 - DNA, 41 bp, Artificial Sequence
  P21_mutant synthetic promoter
  misc_feature (7)..(10): n is a, c, g, or t
  misc_feature (30)..(35): n is a, c, g, or t
tgtgcgnnnnctaacacgtcctagtatggnnnnnngagcaa

SEQ ID NO: 44 - DNA, 40 bp, Saccharopolyspora spinosa
cccggatcgaagcacccacccccactgttactgtgatcag

SEQ ID NO: 45 - DNA, 209 bp, Saccharopolyspora spinosa
aaccactgcgacgagcggtatttgggggaagtaaagaggtgacccacgactcactgtcggtgatcgatatcgacccggaaacgacttgataacgacgctctgatcagcacaaataccccggatcgaagcacccacccccactgttactgtgatcagcgtcacatgatctcaggtttccgatctccgtgttggttacgtagtgtcgctcc

SEQ ID NO: 46 - DNA, 433 bp, Saccharopolyspora spinosa; identical to SEQ ID NO: 37

SEQ ID NO: 47 - DNA, 53 bp, Artificial Sequence
  P21-P1 synthetic promoter
tgtgcgggctctaacacgtcgaagtatggtaggatgagtgttactgtgatcag

SEQ ID NO: 48 - DNA, 52 bp, Artificial Sequence
  P1-P21 synthetic promoter
cccggatcgaagcacccgggctctactgttactgtgatggtaggatgagcaa

SEQ ID NO: 49 - DNA, 332 bp, Saccharopolyspora spinosa
gggcaggcccagcttctcgccgcgccagtcgggccgcgcctcggcggcctgctcgcgcgcgtggttgaatgcgctcttcgggccgtccagccacgaaccgatgactcttcgcacgtcatccagggtacgtgcccctgccatacggccagcggagcatcacgctcggccggtgcgcgcaaccccgaccacccaaacgggcggcagttaacacccacgaaacattcaggtgacgacagggcaacacccctaacataacgtggactacgagccggcggtggaacctttggcgttgcgtcggtgagctgtacgagtgcgtgaaggagccaccgagg

SEQ ID NO: 50 - DNA, 300 bp, Saccharopolyspora spinosa
cacgagcgcgctcgacggaccagctcaccgagaagtaggtcggagcaccgttagcgggaaaagtggggttatcggcgttgcactaagcacgatggaccatttgaggtaatgcgatgtagcccaaccggctggttggcgtgttgatgttgcggttgaatgccgcgttacgcgtcccgggcaaattcgacttaaatgtcgcctgtatcacaattctgttacttctgacggactgtcgcttagagtacctctccgggttcagccagcgataaatagtcgctggctccgtctggggggatggga

SEQ ID NO: 51 - DNA, 247 bp, Saccharopolyspora spinosa
cccggcaattgttgggcccacagcaaaaatcatgctcaagggctcgccctgtaaccgggggacgattgtttgtgggtggtgtgttgtggtgcgggtgccgggtggttgtcctcgcccggtcgctgggttgtggtgtgtcggcgcgctggtgggtcgcggtgcgggcatctagtggggcggaaggcctgatttcggttgctgttggtggtgttctgggttctggcggcgttggggtcgggtgggtgtg

SEQ ID NO: 52 - DNA, 230 bp, Saccharopolyspora spinosa
gctggcggaggggcacgagcgggcgatcgcagcgggtgcgacgccgctgccgcagccgcccgaccagcagggcgcgagcttccgcgtctacgccgaccccgatggtcacccgttctgcatgtgcgcctgcgaggagtgagcgcgctctccggtcaggggcgcaagcagtctgcttgcaggagctagcacttgtggttatcgtcgttggtgaccggagaggtgctagcccg

SEQ ID NO: 53 - DNA, 300 bp, Saccharopolyspora spinosa; identical to SEQ ID NO: 28

SEQ ID NO: 54 - DNA, 300 bp, Saccharopolyspora spinosa
tgtcgccgtgggtagagtgtctggacgaggaagacgtgagctcgcacccaggagatgggcgccaccgcagcgccgggtgatcgagcaggtcaaggggaccccgttttgaccctctgagaacggggcaggtatctttgtttatcggacccgacacccatcgggtcgatccgcatgcccgtgcatgacgtggtcgccagccgctggttgagaccgcgccagcgctgaggacgtgtggaactcctctcaacaaccctctggggtcgttcccattggggcgcatcggcgccgaaaggccgagga

SEQ ID NO: 55 - DNA, 298 bp, Saccharopolyspora spinosa
acccgcgaggcgcccgaaccattgattgcgcaaatttttcacaatccgcgttcgtattacgtcgcttggccacgctccgccgttacggagatagctcatagtcacccaaaagagcgatacgatcatgttcaggtaacaactcgatcgggatagatacccgattgatcgtcccgctgacccgcttgggcggttacgctgcccccgacgacaccactttcggtcgatcagtggcgcggctgattggtcgagagtcccggtgccggtcggggggcacgaccgactccagggaggtagtgac

SEQ ID NO: 56 - DNA, 300 bp, Saccharopolyspora spinosa; identical to SEQ ID NO: 35

SEQ ID NO: 57 - DNA, 300 bp, Saccharopolyspora spinosa
ggtggcacttaccgatccgtcggggacgcgtccttcgagccagtgcttcggtacgccgccgagccagcgcaccggatccggttcgccggtgactacttcgcgccgctggggcagatggaggtagccgtgaccgcgggtaaagacgcggccgaggcggtcatccgtgatcgcgcgggagcgcatcagcgtcgcttcggctcggtcaaacccaggtgaaccccttgacctacctcagggaagcagcgatattcatacgtagacggtagtcgattaccgatcagataccaccctggaggaaga

SEQ ID NO: 58 - DNA, 248 bp, Saccharopolyspora spinosa
ggccggattcctcggcggacaggcgcagccccggctgatcgagaggatcggggtgggggccgaggccgcgatcatcccggcgttcatgggaggacatcggtgtgctcaccgatcccgagcgccgggtgtcgctgatcatgcgcgacggcgtgctggtgaaggaccgcccgacggtgtgatatttgcctgttaaccccgctttcatcccaggtcaagcgcctgcaatacagcgcgggaagcatggtggc

SEQ ID NO: 59 - DNA, 250 bp, Saccharopolyspora spinosa
cggcgcgagcactccagcaccccgagccggggctcgtcggagagcaacagcagcaaccgggcgtccaacgcgtcgagcccctcagcattgggagccatgtcatatcccttgttcaggctgaccaataaagccagcgatttggatcaaatacttatcattttgtgcagcgaaatcaaatactgttgctcaggatgacctgcccggcgcacgctgaccccgtcactgcttcggcgaaacaggggaggacatc

SEQ ID NO: 60 - DNA, 250 bp, Saccharopolyspora spinosa
tgagaccggatcgacgcactttcccacgcctatacgggtatctggcgaatggcggaatctgactttgggttcggcagtgacctggacttatatgtcgatgtgcgcatcagtcgacgtgatactcgcgactaaccgcaggtgatttgccgaacgggtctgcgtatttccctgcggcgagttagggtgcccttgcttgccttgaacattgctctacctcatcaggactccttcgaagggaagtgagctgctc

SEQ ID NO: 61 - DNA, 298 bp, Saccharopolyspora spinosa
tcgctttgacgcggacaccccggcctttttgtcgctcgctagaggtaaaaacgacacacgatcgagcgattaccttttgtgtaacactccaaactcaacggtttccccgacccgtttgtcctgatcaagcggcagatgcgggtgatcgggatcaggtcggtccgcgtcggtggccgcagagtgcgcgttagcgtcccggcggcacttgatctcgcagatcaggctgcccgctgcaggtgcttcccggccttcgccggagctgccgacaacaggtacgccaacgggccaggagctgatc

SEQ ID NO: 62 - DNA, 296 bp, Saccharopolyspora spinosa
caagaagccgaaaggcggcgaactctcggcggcagataagaaaaacaacaaaacgatctcatcgctacgatctgccgtcgagcgatgcatcgcacatttaaagaattggaagatacttgccaccgggtaccgaggacggctcgctgaactctccaacatcatccgcatcgtcacggcgctcgaattctatcgactcggctggtaactcacgtgaataacgctcttcgtgttcagcaaacctgtggaccatcaccaacaccgaaacgtctaatgggcaagtcaaccttcgcggcgaa

SEQ ID NO: 63 - DNA, 300 bp, Saccharopolyspora spinosa; identical to SEQ ID NO: 28

SEQ ID NO: 64 - DNA, 226 bp, Saccharopolyspora spinosa
gggtgatcgagcaggtcaaggggaccccgttttgaccctctgagaacggggcaggtatctttgtttatcggacccgacacccatcgggtcgatccgcatgcccgtgcatgacgtggtcgccagccgctggttgagaccgcgccagcgctgaggacgtgtggaactcctctcaacaaccctctggggtcgttcccattggggcgcatcggcgccgaaaggccgagga

SEQ ID NO: 65 - DNA, 191 bp, Saccharopolyspora spinosa
acggccagcggagcatcacgctcggccggtgcgcgcaaccccgaccacccaaacgggcggcagttaacacccacgaaacattcaggtgacgacagggcaacacccctaacataacgtggactacgagccggcggtggaacctttggcgttgcgtcggtgagctgtacgagtgcgtgaaggagccaccgagg

SEQ ID NO: 66 - DNA, 177 bp, Saccharopolyspora spinosa; identical to SEQ ID NO: 30

SEQ ID NO: 67 - DNA, 299 bp, Saccharopolyspora spinosa; identical to SEQ ID NO: 32

SEQ ID NO: 68 - DNA, 266 bp, Saccharopolyspora spinosa
atcgggacactacgccgcgcactcgcgtgcacggaccactgggctcgttttcccggacaacccgaaaccacgcgccaatttgccgacgcaacgagaactgtgcgacacgacacatccggatcacacgatggtgttaacgaggtgttttttcttcggtttgcctggatatctttcacgccgagtacgccccctcccggctgaacttatggggtgcgcagccggggaaggagcgctctgcttcatcccatccaacaaggagcaacaaa

SEQ ID NO: 69 - DNA, 163 bp, Saccharopolyspora spinosa
ttatatgtcgatgtgcgcatcagtcgacgtgatactcgcgactaaccgcaggtgatttgccgaacgggtctgcgtatttccctgcggcgagttagggtgcccttgcttgccttgaacattgctctacctcatcaggactccttcgaagggaagtgagctgctc

SEQ ID NO: 70 - DNA, 37 bp, Saccharopolyspora spinosa
cccgaaccttcgggggcgggccctcttgcttttcaat

SEQ ID NO: 71 - DNA, 49 bp, Saccharopolyspora spinosa
cgggcaataatacgtgcccggacggtagtgcgagcacgaggtgggtacg

SEQ ID NO: 72 - DNA, 41 bp, Saccharopolyspora spinosa
agtttgtcgaaccggcggcgttcgccggctttaccttgcgc

SEQ ID NO: 73 - DNA, 42 bp, Saccharopolyspora spinosa
ggtttctcgaaccagtgctttgcgtactggttgtcgttgcag

SEQ ID NO: 74 - DNA, 37 bp, Saccharopolyspora erythraea
cggagccagagggcgcctgagtgcctgtttttgatcc

SEQ ID NO: 75 - DNA, 39 bp, Saccharopolyspora erythraea
aaacgcccccggctccggccgggggcgtttttggttgtg

SEQ ID NO: 76 - DNA, 37 bp, Saccharopolyspora erythraea
agacgcaggaggtctcgtgaggggcttttccgcgagc

SEQ ID NO: 77 - DNA, 35 bp, Saccharopolyspora erythraea
cgtgtgacttgtcccactcggggtttttgtcgcga

SEQ ID NO: 78 - DNA, 39 bp, Saccharopolyspora erythraea
ggattcgtccggccgaggccaatcggcttttcggggccc

SEQ ID NO: 79 - DNA, 38 bp, Saccharopolyspora erythraea
gctttcgtcggccgggaacgccctggtgtttcttaccg

SEQ ID NO: 80 - DNA, 38 bp, Saccharopolyspora erythraea
ttgggtggattcacccctaccgggtgtttttctcggct

SEQ ID NO: 81 - DNA, 711 bp, Artificial Sequence
  codon optimized reporter gene DasherGFP
atgacggcattgacggaaggtgcaaaactgtttgagaaagagatcccgtatatcaccgaactggaaggcgacgtcgaaggtatgaaatttatcattaaaggcgagggtaccggtgacgcgaccacgggtaccattaaagcgaaatacatctgcactacgggcgacctgccggtcccgtgggcaaccctggtgagcaccctgagctacggtgttcagtgtttcgccaagtacccgagccacatcaaggatttctttaagagcgccatgccggaaggttatacccaagagcgtaccatcagcttcgaaggcgacggcgtgtacaagacgcgtgctatggttacctacgaacgcggttctatctacaatcgtgtcacgctgactggtgagaactttaagaaagacggtcacattctgcgtaagaacgttgcattccaatgcccgccaagcattctgtatattctgcctgacaccgttaacaatggcatccgcgttgagttcaaccaggcgtacgatattgaaggtgtgaccgaaaaactggttaccaaatgcagccaaatgaatcgtccgttggcgggctccgcggcagtgcatatcccgcgttatcatcacattacctaccacaccaaactgagcaaagaccgcgacgagcgccgtgatcacatgtgtctggtagaggtcgtgaaagcggttgatctggacacgtatcagtaa

SEQ ID NO: 82 - DNA, 792 bp, Artificial Sequence
  codon optimized reporter gene PaprikaRFP
atggtgagcaagggtgaggaactgattaaagagaatatgcgcatgaagctgtacatggaaggcacggtgaataaccaccacttcaaatgcaccagcgagggtgagggtaaaccgtatgaaggcacccaaacgatgcgtatcaaagttgttgagggtggcccgttgccgtttgcgttcgacattttagcgacgagctttatgtatggctctcgtacgtttatcaagtacccgaagggtattccggactttttcaaacaatcttttccagagggtttcacctgggagcgcgtgactcgctacgaagatggcggcgtcgtgaccgttatgcaggatacctccctggaagatggctgcctggtctaccacgttcaggtccgtggtgtcaatttcccgagcaatggtccggttatgcagaagaaaaccaagggttgggaaccgaacaccgagatgttgtatcctgcagatggtggcctggaaggtcgcagcgacatggcattgaaactggtcggtggcggccatctgagctgtagcttcgtgaccacgtatcgttcgaagaagccggcgaagaacctgaaaatgccgggtattcacgcggttgaccaccgtctggagcgcctggaagaatccgacaacgagatgttcgtggtgcaaagagaacatgccgttgcgcgttattgtgatctgccgagcaagctgggccataagctgaacagcggtctgcgtagccgcgctcaggccagcaattccgcggtcgatggtaccgctggtccgggtagcacgggtagccgttaa

SEQ ID NO: 83 - DNA, 1812 bp, Artificial Sequence
  codon optimized reporter gene gusA
atgctgcgccccgtggaaaccccgacgcgcgaaatcaagaagctggacggcctctgggccttctccctggaccgggagaactgcgggatcgaccagcgctggtgggagtccgccctgcaggagtcgcgcgccatcgccgtgccggggagcttcaacgaccagttcgcggacgccgacatccgcaactacgcgggcaacgtgtggtaccagcgcgaggtcttcatcccgaagggctgggcgggccagcggatcgtcctgcgcttcgacgccgtgacccactacggcaaggtctgggtgaacaaccaggaggtcatggaacaccagggcgggtacaccccgttcgaggccgacgtcaccccgtacgtcatcgccggcaagagcgtccgcatcaccgtctgcgtcaacaacgagctgaactggcagacgatcccccccggcatggtcatcaccgacgagaacgggaagaagaagcagagctacttccacgacttcttcaactacgccggcatccaccgctcggtgatgctgtacacgacccccaacacctgggtcgacgacatcacggtggtgacccacgtcgcccaggactgcaaccacgccagcgtggactggcaggtggtggccaacggcgacgtctccgtggagctccgcgacgcggaccagcaggtcgtcgccaccggccaggggacctcgggcaccctgcaggtggtcaacccgcacctctggcagcccggcgagggctacctctacgagctgtgcgtcacggcgaagtcgcagaccgagtgcgacatctaccccctgcgcgtcggcatccggtccgtggccgtcaagggcgagcagttcctgatcaaccacaagcccttctacttcaccggcttcggccgccacgaggacgccgacctccggggcaagggcttcgacaacgtcctgatggtccacgaccacgcgctgatggactggatcggcgccaactcctaccgcacctcccactacccctacgccgaggagatgctcgactgggccgacgagcacgggatcgtcgtgatcgacgagaccgccgccgtcggcttcaacctctcgctcgggatcggcttcgaagcggggaacaagcccaaggagctctactccgaggaagccgtcaacggcgagacccagcaggcccacctgcaggcgatcaaggagctgatcgcgcgcgacaagaaccacccgagcgtcgtcatgtggagcatcgccaacgaaccggacacgcgcccgcagggtgcgcgggaatacttcgccccgctcgccgaagccacccgcaagctcgaccccacgcgccccatcacctgcgtcaacgtgatgttctgcgacgcgcacaccgacaccatctccgacctgttcgacgtcctgtgcctgaaccgctactacggctggtacgtccagtccggggacctggagacggcggaaaaggtgctggagaaggagctcctggcgtggcaggagaagctgcaccagcccatcatcatcacggagtacggggtcgacaccctggccggcctccactccatgtacacggacatgtggagcgaggagtaccagtgcgcctggctggacatgtaccaccgcgtcttcgaccgcgtgagcgcggtcgtgggcgaacaggtctggaacttcgccgacttcgcgacgtcgcagggcatcctgcgcgtggggggcaacaagaagggcatcttcacccgcgaccgcaagcccaagtccgccgccttcctcctgcagaagcggtggaccgggatgaacttcggcgagaagccccagcaggggggcaagcagtga

SEQ ID NO: 84 - DNA, 1740 bp, Saccharopolyspora endophytica
atgccgcgtaagaaccgcgatgaaggcacccgggcgcccaacggcgcgagcagcatctacaagggcaaagacggctactggcacggccgcgtctggatgggcaccaaggacgacggcagtgaggaccgtcgccacaggtcagcgaagagcgaaacagagctcctcaataaggttcgcaagctcgaacgggagcgggacagcggcaaggtgcagaagcctggccgcgcctggaccgtcgagaaatggcttacgcactgggtggagaacatcgccgctcccaccgtgcggccgaccacgatggtcggctaccgcgcctcggtgtataagcatctgatccccggcgtgggcaagcaccggatcgacaggttgcagccggaacacctcgaaaagctctacgccaagatgcagcgcgatggactcaaggccgcgacagcgcacctcgcgcaccggacggtgcgggtcgcgctgaacgaggccaagaagcgacgtcacatcaccgagaacccggccaatatcgcgaagccgcccagggtggacgaggaggagattgtcccgttcacggtggatgaagcccgccggatcctcgcagcagctgcggagacgcggaacggcgctcgctttgtcatcgcgctgacccttggcctgcgcaggggtgaagcactcgggttgaagtggtcggatctctcgatcacctggaagcacggatgccggaaggggagcgcgtgccgggtgggtcgccgagccgagcagtgcggcgagcgtcgcggcagcggcacgctcgtcatccggcgcgcgattcagcagcaggtttggcagcacggttgctcagaggacaagccgtgcgaccaccgctacggcgctcactgcccgcgccggcatagcggcggtgtggtcgtgaccgatgtgaagtccagggcgggtcggcgaaccgtgggccttccgcacccggtggtggaagcgctcgaagagcaccgcgcccgccagcggacagagcgggagaaggcgcgcaacgagtgggacgacgccgattgggtcttcacgaacaggtggggtcgcccggttcatccgaccgttgactacgacgcctggaaggcactgctcagggcagcgaacgtgcgcaacgcgcggttgcacgacgcacgccacaccgcggcgacgatgttgctggtgttgaaggttccgctgcctgcggtcatggaaatcatgggctggtcggaagcctctatggccaagcgctacatgcacgtgccgcacgagctcgtgaccgcgatcgcggaccaggtgggtgacctggtgtggcccgtcccagagaccgaggaggaggcgccaccgcctgaggaggagtgggcgctggacgccaaccaggtggcggcgatccggaagctggccggagctctcccgccgcagttgcgggagcagttcgaggcgctgctgcccggcgacgacgaggacgacggcccgacttcgggagtggtcatccctgcgtaaccagtgcggccagaacccggcctaacggggcctactgagacgaaaactgagactggacatgcgagaggcccggaagcgagatcgcttccgggcctctgacctgcggaggatacgggattcgaacccgtgagggctattaacccaacacgatttccaattccgatggcgcgagtgccagggggtagctgaacgtgccttttgcctggtcagtggcactacggcaacatcaggtgtggcttgatccgtgcgcgt

SEQ ID NO: 85 - protein, 495 aa, Saccharopolyspora endophytica
MPRKNRDEGTRAPNGASSIYKGKDGYWHGRVWMGTKDDGSEDRRHRSAKSETELLNKVRKLERERDSGKVQKPGRAWTVEKWLTHWVENIAAPTVRPTTMVGYRASVYKHLIPGVGKHRIDRLQPEHLEKLYAKMQRDGLKAATAHLAHRTVRVALNEAKKRRHITENPANIAKPPRVDEEEIVPFTVDEARRILAAAAETRNGARFVIALTLGLRRGEALGLKWSDLSITWKHGCRKGSACRVGRRAEQCGERRGSGTLVIRRAIQQQVWQHGCSEDKPCDHRYGAHCPRRHSGGVVVTDVKSRAGRRTVGLPHPVVEALEEHRARQRTEREKARNEWDDADWVFTNRWGRPVHPTVDYDAWKALLRAANVRNARLHDARHTAATMLLVLKVPLPAVMEIMGWSEASMAKRYMHVPHELVTAIADQVGDLVWPVPETEEEAPPPEEEWALDANQVAAIRKLAGALPPQLREQFEALLPGDDEDDGPTSGVVIPA

SEQ ID NO: 86 - DNA, 1525 bp, Saccharopolyspora erythraea
atgccccgcaaacgccgcccagaaggcacccgagcccccaacggcgccagcagcatctactacagcgagacggacggctactggcacgggcgcgtcacgatgggcgtccgcgacgacggcaagcccgaccgtcgccacgtccaagccaagaccgagaccgaggtcatcgataaggtccgcaagctcgaacgtgaccgggatagcggcaacgcgcggaagcctggtcgcgcgtggacagtcgagaagtggctgactcactgggtcgagaacatcgcggtgcactccgttcggtacaagacgcttcagggctaccgaacggcggtctacaagcacctgatccccggtatcggcgcgcaccggatggaccgcatcgagccggagcacttcgagcggttctacgccaggatgcaggccgccggcgccagtgcagggaccgcacatcaggtgcaccggactgccaaaacggcattcaacgaatacttccggcggcagcgcatcaccgggaaccccatcgccttcgtgaaagcgccgcgcgtcgaggaaaaggaagtggagccgttcacgccgcaggaagccaagagcatcatcacggccgcgctcaagcggcgcaacggcgtgcgatacgtcgtcgccttggctctcggttgtcgccaaggcgaagccctggggttcaagtgggaccgcctcgaccgcgggaaccggctttaccgcgtacggcaggcattgcagcggcaggcttggcaacacggatgcgacgacccgcacgcctgcggagcacgacttcatcgggtggcgtgcccggacaactgcacccagcatcgcaaccgcaagagctgcattcgcgacgagaagggccaccaccgtccgtgcccgccgaactgcaccaggcacgcgagcagttgcccgcagcggcacggtggtgggctcgtcgaggtcgacgtgaagtcgaaggctggtcgccggagcttcgttctgccagatgaggtcttcgatctgctgatgcgccacgagcaggcgcagcagcgggagcgcaagcacgccggtagcgagtggcaggaggggggctgggtcttcacccagcccaacggccggccgatcgatccgcggcgcgactggggtgagtggaaggacatcttgggggaggcaggtgttcgggatgctcggctgcacgacgcgcgccacactgcggcgacggtcctcatgctgctccgcgttccagaccgggccgtccaggatcacatgggctggtcctcgatccggatgaaggagcgctacatgcacgtcaccgaggaactgcgacgagagatcgccgatcagctcaacgggtacttctgggacgtcaactgagacggaaagtgagacgaaaagcgcctggtcagggacctgtcgacggcgtttccgctggtagtttcggagccgctgaggggactcgaacccctgaccgtccgcttacaaggcgggcgctctaccaactgagctacagcggcgtgcgctacgtcgcgcgcgaacatcgtaagcgtccacc

SEQ ID NO: 87 - protein, 448 aa, Saccharopolyspora erythraea
MPRKRRPEGTRAPNGASSIYYSETDGYWHGRVTMGVRDDGKPDRRHVQAKTETEVIDKVRKLERDRDSGNARKPGRAWTVEKWLTHWVENIAVHSVRYKTLQGYRTAVYKHLIPGIGAHRMDRIEPEHFERFYARMQAAGASAGTAHQVHRTAKTAFNEYFRRQRITGNPIAFVKAPRVEEKEVEPFTPQEAKSIITAALKRRNGVRYVVALALGCRQGEALGFKWDRLDRGNRLYRVRQALQRQAWQHGCDDPHACGARLHRVACPDNCTQHRNRKSCIRDEKGHHRPCPPNCTRHASSCPQRHGGGLVEVDVKSKAGRRSFVLPDEVFDLLMRHEQAQQRERKHAGSEWQEGGWVFTQPNGRPIDPRRDWGEWKDILGEAGVRDARLHDARHTAATVLMLLRVPDRAVQDHMGWSSIRMKERYMHVTEELRREIADQLNGYFWDVN

SEQ ID NO: 88 - DNA, 2172 bp, Saccharopolyspora erythraea
acgtcacccaactcgccgccacgctcgcctcgctcgcggccctgctcgccgaacagcagcccgccccggaacccgagcccgaaccggccgcccgcaggctgcccaaccgcgtgctgctcacggtcgaggaagcggccaagcaactggggctcggcaggaccaagacctacgcgctggtggcgtctggcgagatcgaatctgtccggatcggtcggctcaggcgcatcccgcgcaccgccatcgacgactacgccgcccgactcatcgcccagcagagcgccgcctgaagggaaccactatggaacaaaagcgcacccgaaaccccaacggtcgatcgacgatctacctcgggaacgacggctactggcacggccgcgtcaccatgggcatcggcgacgacggcaagcctgaccggcgccacgtcaagcgcaaggacaaggacgaagttgtcgaggaggtcggcaagctcgaacgggagcgggactccggcaacgtccgcaagaagggccagccgtggacagtcgagcggtggctgacgcactgggtggagagcatcgcgccgctgacctgccggtacaagaccatgcggggctaccagacggccgtgtacaagcacctcatccccggtttgggcgcgcacaggctcgatcggatccagaaccatccggagtacttcgagaagttctacctgcgaatgatcgagtcgggactgaagccggcgacggctcaccaggtacaccgcacggcgcgaacggctttcggcgaggcgtacaagcggggacgcatccagaggaacccggtttcgatcgcaaaggcacctcgggtggaagaggaggaggtcgaaccgcttgaggtcgaggacatgcagctggtcatcaaggccgccctggaacgccgaaacggcgtccgctacgtcatcgcactggctctcggaactcggcagggcgaatcgctcgcgctgaagtggccgcggctgaaccggcagaagcgcacgctgcggatcaccaaggcactccaacgtcagacgtggaagcacgggtgctctgacccgcatcggtgcggcgcgacctaccacaagaccgagccgtgcaaggcggcctgcaagcggcacacgcgagcttgtccgccgccatgcccgccagcttgcaccgaacacgcccggtggtgcccgcagcgaaccggtggcgggctggtcgaggtcgacgtcaagtcgagggctggacgacggaccgtgacgctgcccgaccaactgttcgacttgatcctcaagcacgaaaagcttcagggggccgaacgggagctcgcgggcacggagtggcacgacggcgagtggatgttcacccagcccaacggcaagccgatcgatccacgtcaggacctcgacgagtggaaagcaatccttgttgaagccggagtccgcgaggcgcggctacatgacgcacggcacaccgccgcgactgtgctgttggtcctcggagtgcccgaccgggtcgtgatggagctgatgggctggtcgtccgtcaccatgaagcagcggtacatgcacgtcatcgactccgtccggaacgacgtagcggaccgcctgaacacctacttctggggcaccaactgagacccagactgagacccaaaacgcccccgtcgagatcgacgggggcgttttggcagctcttggtggtggccaggggcggggtcgaaccgccgaccttccgcttttcaggcggacgctcgtaccaactgagctacctggccgttcgcgcccggctcaaagccgaaccgctgtggcgacccagacgggactcgaacccgcgacctccgccgtgacagggcggcgcgctaaccaactgcgccactgggccatgttctgttgttgcgtacccccaacgggattcgaacccgcgctaccgccttgaaagggcggcgtcctaggccgctagacgatgggggcttggccgattcggaaccgacccggcctcgcctccaaccggctttccctttcggggcgccccgttgggagcagtgaaagcttacgacacaccccccagcgccccacaacgggggggtccccaaacctcacgagcccccgcgcggcccacgcccgccggtcacgtcggtcgccaccatatgccatctgaccagccttttccatcgcctatcctcagtcggcccact

SEQ ID NO: 89 - protein, 437 aa, Saccharopolyspora erythraea
MEQKRTRNPNGRSTIYLGNDGYWHGRVTMGIGDDGKPDRRHVKRKDKDEVVEEVGKLERERDSGNVRKKGQPWTVERWLTHWVESIAPLTCRYKTMRGYQTAVYKHLIPGLGAHRLDRIQNHPEYFEKFYLRMIESGLKPATAHQVHRTARTAFGEAYKRGRIQRNPVSIAKAPRVEEEEVEPLEVEDMQLVIKAALERRNGVRYVIALALGTRQGESLALKWPRLNRQKRTLRITKALQRQTWKHGCSDPHRCGATYHKTEPCKAACKRHTRACPPPCPPACTEHARWCPQRTGGGLVEVDVKSRAGRRTVTLPDQLFDLILKHEKLQGAERELAGTEWHDGEWMFTQPNGKPIDPRQDLDEWKAILVEAGVREARLHDARHTAATVLLVLGVPDRVVMELMGWSSVTMKQRYMHVIDSVRNDVADRLNTYFWGTN

SEQ ID NO: 90 - DNA, 1521 bp, Saccharopolyspora spinosa
atgccacgcaaacgccgcccggaaggcacccgggcacccaacggagccagcagcatctacctcggcaaggacggctactggcacggccgcgtcaccgtcggagttcgcgacgacggtaagcccgaccgccctcacgtccaggccaagaccgaggccgaagtcatcgacaaggtgcgcaagctcgaacgcgatcgcgatgcggggaaggtgcgaaagcctggccgggcctggaccgtcgagaagtggcttacgcactgggtcgagaacatcgccgcgccatccgtccgttacaagacccttcagggctaccgcacggcggtgtacaagcacttgatccccggcatcggcgcgcaccggatcgaccgaattgaaccggagcacttcgagaagctctacgcgaagatgcaggaatccggcgcgaaagcgggaaccgcgcaccaggtgcaccgcaccgctcgggccgcctttaacgaagccttccggcgtcggcacctcaccgaaagcccggtgcggttcgtgaaagcgccgaaggtcgaagaagaggaagtcgagcccttcacgccgaaggaagcccagcagatcattacggccgcgctcaatcgtcgaaacggcgtgcgattcgtgatcgctctcgcactgggctgccgccagggtgaagcgctgggcttcaagtgggaacggctcgaccgggaaaacaggctctaccacgttcggagggcgcttcagcgtcaagcctggcaacacggctgtgaagatccgcacaactgcggtgcgaggttccaccgggttgcttgcgccgagaactgcaagcggcaccgcaatcggaagaactgcattcgcaacgagaagggacacgctcgaccgtgcccgccgaactgcgaccgacacgccagcagctgcccgaaacggcacggcggaggcctgcgcgaggtggatgtgaagtcgaaggctggccgccggcggttcgttcttcctgacgagatcttcgacctgctcatgcggcatgaggaagtccagcggcacgaacgggttcacgccggtaccgagtggcaggagggcggctggatcttcacgcagcccaacggcaggccgatcgatccgcgccgcgattggggcgagtggaaggagatcctcgcggaggccggtgttcgggatgcccggctgcacgacgcgcggcacaccgcagcgacggtgctcatgctgctccgtgttccggaccgggccgttcaggaccacatgggatggtcgtcgatccggatgaaagagcggtacatgcacgtcaccgaggaactgcgccgcgagatcgccgatcagctgaatgggtatttctggaaccccaactgagaccgaaagtgagacggatcgcgcctggtcaccgggtgggcaggcgcgtttccgctggtacggtcggagccgctgaggggactcgaacccctgaccgtccgcttacaaggcgggcgctctaccaactgagctacagcggcatgcacttcgtcgtgcggggacatcgtaagcggcgat

SEQ ID NO: 91 - protein, 447 aa, Saccharopolyspora spinosa
MPRKRRPEGTRAPNGASSIYLGKDGYWHGRVTVGVRDDGKPDRPHVQAKTEAEVIDKVRKLERDRDAGKVRKPGRAWTVEKWLTHWVENIAAPSVRYKTLQGYRTAVYKHLIPGIGAHRIDRIEPEHFEKLYAKMQESGAKAGTAHQVHRTARAAFNEAFRRRHLTESPVRFVKAPKVEEEEVEPFTPKEAQQIITAALNRRNGVRFVIALALGCRQGEALGFKWERLDRENRLYHVRRALQRQAWQHGCEDPHNCGARFHRVACAENCKRHRNRKNCIRNEKGHARPCPPNCDRHASSCPKRHGGGLREVDVKSKAGRRRFVLPDEIFDLLMRHEEVQRHERVHAGTEWQEGGWIFTQPNGRPIDPRRDWGEWKEILAEAGVRDARLHDARHTAATVLMLLRVPDRAVQDHMGWSSIRMKERYMHVTEELRREIADQLNGYFWNPN

SEQ ID NO: 92 - DNA, 1669 bp, Saccharopolyspora spinosa
atgccacgcaagcgccgcccggaaggcacccgggcacccaacggagccagcagcatctacctcggaaacgacggctactggcacggccgcgtcacgatgggaacccgtgacgacggccgccccgaccgacggcatgtccagggcaagaccgaggccgaagtcatagacaaagtgcgcaagctcgaacgcgaccgcgacgccggacggatgcgcaagcctggccgggcctggaccgtcgagaagtggctgatgcactggctggagcacattgcgaagccatcggtccggccgaaaaccgtcgcccggtatcggacttccgtcgagcaatacctgattcctggtctcggtgcgcaccgcatcgaccgcttgcagccggagaacattgagaagctgtacgcaaaattgctcgctcgcgggttggcgccgtccactgtgcaccatgttcaccggactctgcgcgtcgctttcaacgaggcgttcaagcgggaacacatcacgaaaaacccggtcctcgttgcgaaagcgccgaagctggtcgaaccggagatcgagccgttcaccgtggccgaagcacaacgaattctcgatgttgcacggacacggcggaatggtgctcggttcgcactcgcgctcgcgctgggaatgcgccagggcgaagctctcggactcaagtggtccgacctgcgaatcacctggcaccacgggtgcgcatccggactcaccgaagaacagcaggcggccatcgaaatgctcgcgaaggtcgatccgcagcgatggaagcggcctgacgattccgggtgcggattcaaggacgtggaggactgcccgcaggctcacccggccgcgacactgaacattcggcgcgcattgcagcgccacacctggcaacacgggtgcggtgacaaaccgacgtgcggcaagaaacggggcgcggactgcccgcagcgtcatggcggcggcttggccatcgtcccggtgaagtcgagggcggggacgcgctcgatcagcgtgcctgagccgctgattcatgcgttgctcgatcacgacgaggcgcaggatgaggaacggcacttggcccggaacctgtggcacgacgatggatggatgttcgctcagcccaacgggaaggcgacggacccgagggccgactatggcgaatggcgcgagctgctggacgccgcgaaggttcggccggcgcggctgcacgacgcgcggcacaccgccgcgacgatgttgctggttctcaaggtcgcaccacgggcaatcatggacgtgatgggctggtcggaggcgtcgatgctgacccgctacgtccacgtgccggacgagatcaagcagggcatcgcgggccaggtcggcggactgctgtggaaggactggcagcagcccgacgacggcccagacgacgaggacggcggcaccgccgggcaccctgtcccggcctgacgtgcccactgccagaggaggcgtttgagccggaaactgagccggaacgacaccaggcgctttccgtgtccacggaaagcgcctggtgagagcggagccgcctaagggaatcgaacccttgacctacgcattacgagtgcgtcgctctagccgactgagctaaggcggcgttgcacggccaagtgtagcgggccggacctcgccgtcgttcatggccccgact

SEQ ID NO: 93 - protein, 481 aa, Saccharopolyspora spinosa
MPRKRRPEGTRAPNGASSIYLGNDGYWHGRVTMGTRDDGRPDRRHVQGKTEAEVIDKVRKLERDRDAGRMRKPGRAWTVEKWLMHWLEHIAKPSVRPKTVARYRTSVEQYLIPGLGAHRIDRLQPENIEKLYAKLLARGLAPSTVHHVHRTLRVAFNEAFKREHITKNPVLVAKAPKLVEPEIEPFTVAEAQRILDVARTRRNGARFALALALGMRQGEALGLKWSDLRITWHHGCASGLTEEQQAAIEMLAKVDPQRWKRPDDSGCGFKDVEDCPQAHPAATLNIRRALQRHTWQHGCGDKPTCGKKRGADCPQRHGGGLAIVPVKSRAGTRSISVPEPLIHALLDHDEAQDEERHLARNLWHDDGWMFAQPNGKATDPRADYGEWRELLDAAKVRPARLHDARHTAATMLLVLKVAPRAIMDVMGWSEASMLTRYVHVPDEIKQGIAGQVGGLLWKDWQQPDDGPDDEDGGTAGHPVPA

SEQ ID NO: 94 - DNA, 643 bp, Saccharopolyspora erythraea
ggtcggatctcccggttactacaggcacaatggccacggctggccatgcttggtctggtcaggtgtcccactgttctcaagccgcagccgctcagccgacaccgagcggtgtgcatggcacctgaacgcgtcaggcagtgacatttgcccaagcgacatgcccgctcgtgaagcgggggactgtacgagagtacgcagaggccttcacccggtcgaatccggggtgccttcggccgcttcgacgcgggctcggccagtagcctgccacaccgccgatcttggcgcaccgcgatggggccgcttgtcgcccgacgagcgccttgctagcgtgcgcccctgcgatccggcccggcgaggtgggagtcaccgggccccgcgaggtgtgcctgacctcggtcgcagccgtgctgccaggcaggtggggcgttccgggggacccacagcggaacgatcgagtactctgtcgtaccttcgtacacagttgtggacaactctgtggacacctgtgtgggcaggtgcagtcagggactgacccgcaaggcgcagcgctcgccgatcccgctcgtgactcgtcgagtgcggtgtccggcctgggcagcagcgacgcaggtcattgcgcaggcgaggggaggggagcacaccg

SEQ ID NO: 95 - DNA, 11001 bp, Saccharopolyspora erythraea
tctccggtcgcgtcttccgcagggtcggcgcgccgggcggcgggccgggtccgggcgccttcctcgtgccggcggtggacgcttacgatgttcgcgcgcgacgtagcgcacgccgctgtagctcagttggtagagcgcccgccttgtaagcggacggtcaggggttcgagtcccctcagcggctccgaaactaccagcggaaacgccgtcgacaggtccctgaccaggcgcttttcgtctcactttccgtctcagttgacgtcccagaagtacccgttgagctgatcggcgatctctcgtcgcagttcctcggtgacgtgcatgtagcgctccttcatccggatcgaggaccagcccatgtgatcctggacggcccggtctggaacgcggagcagcatgaggaccgtcgccgcagtgtggcgcgcgtcgtgcagccgagcatcccgaacacctgcctcccccaagatgtccttccactcaccccagtcgcgccgcggatcgatcggccggccgttgggctgggtgaagacccagcccccctcctgccactcgctaccggcgtgcttgcgctcccgctgctgcgcctgctcgtggcgcatcagcagatcgaagacctcatctggcagaacgaagctccggcgaccagccttcgacttcacgtcgacctcgacgagcccaccaccgtgccgctgcgggcaactgctcgcgtgcctggtgcagttcggcgggcacggacggtggtggcccttctcgtcgcgaatgcagctcttgcggttgcgatgctgggtgcagttgtccgggcacgccacccgatgaagtcgtgctccgcaggcgtgcgggtcgtcgcatccgtgttgccaagcctgccgctgcaatgcctgccgtacgcggtaaagccggttcccgcggtcgaggcggtcccacttgaaccccagggcttcgccttggcgacaaccgagagccaaggcgacgacgtatcgcacgccgttgcgccgcttgagcgcggccgtgatgatgctcttggcttcctgcggcgtgaacggctccacttccttttcctcgacgcgcggcgctttcacgaaggcgatggggttcccggtgatgcgctgccgccggaagtattcgttgaatgccgttttggcagtccggtgcacctgatgtgcggtccctgcactggcgccggcggcctgcatcctggcgtagaaccgctcgaagtgctccggctcgatgcggtccatccggtgcgcgccgataccggggatcaggtgcttgtagaccgccgttcggtagccctgaagcgtcttgtaccgaacggagtgcaccgcgatgttctcgacccagtgagtcagccacttctcgactgtccacgcgcgaccaggcttccgcgcgttgccgctatcccggtcacgttcgagcttgcggaccttatcgatgacctcggtctcggtcttggcttggacgtggcgacggtcgggcttgccgtcgtcgcggacgcccatcgtgacgcgcccgtgccagtagccgtccgtctcgctgtagtagatgctgctggcgccgttgggggctcgggtgccttctgggcggcgtttgcggggcatggatggttcctctcggtggtcggtttcgtggctggttgtgggcgcgcggggtcgccgcgcgagagcgcggtggggggttagatccggagttagatcggcggaggcgagttagatatctaactcgggggttagatcgttctgctgctgggctgacctgcggaaagttaggcaagttagccgagttacggggttagcgcccaggttgggagcagcctgggaggctcccaactcggggtgagcgagggtgccaggtggctaactcgtctaactcgtctagcgcttcgcccgggtcgccgagcgggccgcgttcggtggcgtggtgggacgctggcccctgtcccgctaggcgctagctcgctggtcggtgccggtccgggccgctagcgctcccgctaggtctagcgggtgtgggcgctaggtctagcggtgcggagccggggtgtgggtacggcggtgccggcgcggtcgagggcgagcatgaggccgcggagtgcggccagtgcggcgaagccaccgagggcgagggtgcgcaccatcccgagggtcaccgcgagcgcagcgatgaggatcacgggcagcaccgcccgccacagcaggacggctaccgcggcggcggtgagcagggcgagggcggcgagcacggccagcacgcggccggtgatgtccagcggggtcatcgtgggtctcccgggtgtggttgtgggcggtcggttcgaacggggtgggtggacggtgcggactgcccggcaccgccgcgcagaggtgtttccgggcggggtgttcacccggtccacctgtccacctgggcggggttgggcctggtggcgggtggtggagtgagtgggtgggcgcgtgggtgggaatccacccggaatcgcggcctccacccctgatccaccgggcatccacttgcccacttgtggggcgttctcgcaggccgtgcccagtgggccgggccagtggatggccagtggatatccactggcccctgccgggctcactggcccgttcactggcctcgcgcggcccggcggtacggggtgagggggtagccgtcccagccgtcccaagcgtcccagtgctggtcaggctgggacggcttgcaggcgtgggacggctcgtgccgtcccagcagtgcgcgccgctggggtcggtgggacggctcgtgccgtcccagggcgtgttgccgtcccgcgctgacctgcggtgggacggctgggacggctgggacggcaccccctcccaccccgatattgggccgactgggcgacgtgacggcggtggtcatgccgcgccccgggccaggcggtcgatgtaggcggtgagcgcttcgacggggacgcggcgggcgtggccgatgcgtaccgagtcgacgtcgccggacttgatgagcttgaacatcgtggtgcggccgaccgacaaccggcgggcggcctgctcgacggtcagcagcagccgcgttgcgtccccggactcccggggccgggggagggaagcgggggccggtgtcgacaaccccggattccgggggtgtcgggcggtgtgcgcggggcgttccctcgttccctgttgtggggtttgcgcaggtcgagccgagggagtggcggagggaacggggtagggagaattccctgcctccccggcggttccctgccgggttccctgtgccgtgacggcggtcgggcggtgggcggtgtgcatggcggctcctgtggggacgaagttgacgaaattacgaactttggcgctgacctggggtttcgtggggagcaaagttcgtagctacgaactttgctttcagggtttgccctggtcaggggcattgttcgtaagttcgtggtgttcgtaggcaatgaagatctcggtggcgcggcctcccttccggccctccgggcggcgctgggtcttggcgatggtgtggtcggcgatgagctggtccagcagcgcttcgatgcgggtcttcctggcgttgccctggaagaagcctgcggtgatctcggtgcgggtgcgtccctgtggcccggcctcgcggatgtaggcggcgagctgggccagcgccgggtcggcttggcggaacaccgcgcgggcggagtcgacggcgtagcggatgaacgccgccgcggcgaccaggtgcgcgggctggatggtgggggtgccgtcgagggcggcgtggatgccggccacccggaggcagttgggtgcggcgcgggagaggaactgctcgatcggtccatcttcggtgctgaagccgccgaactcgacgtagagccgccgccacaggtaggcggcctcgtcgctgaagccgagctgtccgagcgtggcgccctggtccaggcgtgcccgcaggtcgaccgccaggcgctcgatcagcgccggatcggcccccgtgcctgccgggaggaactgggtctgggcgacgaagaccgggagaaaccggttgtaggtgcctccggccatgtcggagtggctgaccttggcgtggaactcgccgggggtgatgtgggtcaggatgccgacgtgggcgtcgcgcacgatgcgggcggtgacgccgagggtggacaggttgccgccctcccacgccgcgcgcagcgtcgccgagagggtgttgccctcgcggcgcatccgcgccatcaccgaggcccactccggttcgaaggccagcagtcgccgatccccggaaggcagcagtgcgcgggggcgcggcttgcctttgccgggctcggtcccgtccccctcgtcttcgtcggcgaacgcctgggtcaggccctcgccggaggtcaggccgctgtggatgttggaggccacgaacccggaatcggcggccgtcaggaggcgtttggcggccgaccagcccgcgcccttgcggccgatgccggtccggccgatgaccatcggccacaccagcagcgggtgccggtcgtcgccgacccgaatgtgtgggcgtccaccgaggtggaccccggttccggccagcagggaggccaggatgttggtggggtcggcctcgctggtcggctgcacctggcggaccaggtcgccgaggaaggtctcgaacatcgcctcatcgcgggcggggagttcggcgcgggcgctggcgatctgagcgagcgcttggcgttcggtttccgggtccggcggcaacatcggtcccgtccctgtgccgggagcctccgggctcgctgtgggctggtcggagtcggagaccaggcgcagccgccgaggctgttccgctggggtggctgcggggtgttcgggcttgctcatgggctaccgctccgatgggtcgtgagtgctgttcagaggggttgggaacccgggtccgcacccgggttcacggcttctgcgccgccttctccggcgggttctcgctgccgttctcggtgatcttggtgaggttcttcccatgcaggtgagaagcggtgagaacccggaggaatcgacctcgattcgtgcaggccaaccaggtggatcgaggccgatcgagtcccggtcgatcgacccccgatccccgctcggctgggtcgttgccgggtctccgggcgactactggcggtgacgctgcggcggtgctcgggtcgggttcactgaggctggaacagtggggggcgggaatccgggtcttaaacccgcaaacccgcaaaagggggtgtgacctggtgttttgtggtgcgggtttggtgcgggtttgcgggtttggtgggctgggtttggcgggtttgcgggtttggtgacggctgtggcgggtttggctccccctcctgtcccggcttctttttcctggtcattggggttggttgcggggtttgcgggtttgcgggtttgctacggcgcttggcgatgtgccgggcggcgtgcccgtggtgacgggcagggcgccggtcacgccgcgtagtgcgaacggtctgcctcgggtccgcagcccgcgttcgctggtggtttcgggctcccaacccgcgcggagttgggagcacccccgtcgaacgcggcgggtcccggggcggctggtctggaggggtgggagacccggcccggtggcggcggttcaaggcccacacggggtgggtggcgggatggcgccaccgggccggggtggtcgggatcggcggggaggaccggcgccggtagtggcttctggccccacacggggtggggtggcggttgcgctaccgggccggggtttgagctgttcgcgaacctgtcgggacgcttccgacagggcaaaacacgccctgtggctgcgaccagcaggaacagccgtttccgctgttcccggcgggtgtgcctgcgaccagcggaaacagtgtgtttgcgctgttccctgggggtgtggccgcgaccagcgggaacgcccgggctagttcggccagatcgcgtccggctcgagctccaccgggtgcaccgccggggtgtcccgtcccggtgccggcgggcgggtgaccgcccggaccgcgtcggcccgggcctgagccagggcgtagcggcgccggtcgaacgtcggcccaccggaggcggccagcaccgccagcccgttctccagccgctcgatcgccttgcggggcgtcttgcggtggaccagggccacgaaccgagccaggtcgtagacctcggcgtcgcgcagctgcgcgtcggtcagcgggcaggcgtagagcacggtcaccacgccatgcacggtcaccttgggcacggtcatcacgcggccaccccttccagctcgatgcgctcggcgccggacaggccgccgatgagcgggtccttcgggctggagaccacctgtggcgtcggggcgccgcgctcgatgtcggcgcgggtgaccaccgggtggttgcgggaccaagcgcccgcgatgcccagcatgcccggggtggtgccgtagacctcggcccagagctgctggtaggtcgacgagaccacccgcacgccgcgttcccactcgccgagcagcccgagcagggtgcccggcgagtgcgcccggatgccccgggaccgggccgcttcggccaggtgctggcaggcggtcttctgcgaccagccccgggctgtgcggtactgccgcagtcgcacgctcatgtgcctcacgccctcgtcagtgcgcggcgcggtgcgggcaccccggggcggccgtagagctcggcgacccggcggcggatcatcatcagcagcgcgtagtccccgctgcccggcttgatgtcctcgatctcttcgcgggcgaggcagatgcggttcgagccctcgtgcaggaaggtctccagcaccatctcgtgtgcgtactggtcgttgccgagttcgcgctcgtccagcaacatcccgccgttgacggcgatccagaacacggcctcgatgtcttccagcgacaccggcacgctcatgctcacctgcatcacgatcggggtgtcgttgtcctcgcggtgggtggggatgaacgtcgcgtggtggtcgccgaccatgccctgtgcccgctcggtgtgcagtcgcatcgtcacgccacctcccccgcggtctcgtcacggtcgtcggcggcggccgactcggtgccggtcacgaacgcctcgtaggcggcctggccgcactgctccaagtagatgttcagggcggagccctcgattcggagggtgcccttgccggtaccgagcttgagggcgtcgagcttcccggactcgatcgcccggtagatggtggccttggagatgttcagcaggtcggcgactgccttgaccttgaacacctggttatcctcgaagcgcatcgtttctccatcggtttcgatgtgcgccctggtcggtgttgcagcactgaccagggctttttcttgtccggctgaggcggttgaggcatctgcaacaaccggaacgagagcaacagtagcgacgagagttaagtgagtcaagtctctgtcgtgacttccttgagttgacgatcgcgtacccttgctgacgtggcaaaacgcgaggtagagaactctcaggcgccctaccgtcaggtcgccgacgccatccgcgcgagcatcacgaacgggacgtacgggccgggggagaagctccctaccggtaaggacttggctgacgagtacggcgtggctatcaacaccgcccgcagcgcgctggaaatcctgcggcaggaggggctgatcgctgtccgccatggccaggggtcgtacgtcctcagccagccggagtccggcggagccgatgccgagaacgctgacgtgcccagcgtcgcggcggtgcatcaacagcttgcggagatcaaccggcgactcgcggcgatcgaggaacgcctgaacgaacttgcccgctgacctgccagcagctacccggcaactgcaccgaagaacgtcttgagcgcgccgaacgtggtcttcacgaactccgcggattcggtcggctgctgcacgaccataaccacgaccacgatcagcacagcccaaccgaacagcttcgggagcaccccgccgccggtcttcatcatcggaaacgactgcttggccatgaccttctccctcgttcagagtgttgactgctttcgctgtcatgcctcaagaatcgatcgactttgcgatgatggggagatgagtcgcgggtgagtcggaggtgagtcgttcgactcacctggctctgaccagggggaacgccaggggagggaccgtgcccgtagaggaacccgaagcaacgcctttgaagcgcgcacgtctcgcgagtggcatgtcgcagatggagacccgcaccaagctccgcgaagtccgacgtcgtcgcggcaagatgcccccgaaagacgtgagcctgaagcggatgtacacggagtgggagcagggtcgcgtccttccgaccgactggcgcgacgaactatgcgaggtcttcgcgctcccgccagcggctctaggcttcgtggacacggcgccgccgccgtctgcattggacattccatcggcgctggagatcaccaggatcgacgccgagatcgtcgaaatgttggagcagcagaccgaccactaccgtctcatggaccggaaggtcggcgcggcgatcatcccgcagaccgtcgcacatgttgagcacatggagaagctgttacgcactgccctgcctggaaagcactcccacctggccgcggtcgcacttgcggagggcgcagccttggcgggctggcaggcgctcgatgcaggtgacgtcacgaaggcgtggaacctgcacgacgtcgcgaaagctgctgcccggcaaggtgaagaccctgctgtgttcgctcacgtgacggctcagcaggcgtacgcgcttctcgacgccggccgggccaccgaagcagtcgagctagtccagtacgcacacacccccgaaatcgcgcggcgtgtcccggctcggcttcgtgcctggcttgcagctgcggaggcggagtttctcgctgccgctggccagcaccgccaggcgctcacgatgctcgatcaggcggccgacgccctacccgaaggcgacaccgatccggaactgcctttcctgatgctgaacagcacgcacctcgcccgttggcgtggtcactgcctcgccaggctaggcgctgacgaggcagtcgatgaccttacgagagccctggagggtagtcaggtcttgtcctcgaagcgggcggagtcgggccttcgggtcgatctcgccttagcgctgcggaagcgtggcgacatcgaaggttcgaaaacgcatgcgcagagagccgcggaactggcgggcagtacgggatcagcacgacagcgcgcacggatcgcgaagctgctcgtggactagctcacctgccgagagccaagtagtggagaaggccgacgagggtgccggagttcttcacctttccctcgcggatcaggttgggcacgttggccacggggacccactcgaacttgccttcgttgacctccgtggcgtctccgaccagctcagcgccccggccgagaaagacgtggtgaggattgcgcagcatgcctactgctggttcgaatgtgactagtggttcgatctttcgaggcttatagccggtttcctcgatcagctcccgcaacgctgtctcctgcgggtcttccccctcgtcgatgatccctccggggagctcccagttccagatatccggcgcgaaccggtgccgccatgccatgagcacatgctcttctgcgtcgtcgagaagcacggtcatcgcaggaggtggaaaccacacagtgtggtgctcgaagcgctctcctgaaggttgcgagatatcggccaagccgacgcgtacccacgcgctctcgtacacaggacgctcaccatgcacggtccacgtgccctcttggtcagccactgatcagcccttctctcggactatcgaaagttcgttctcttggtgttccccggtgcagctagcgagaacggtaggcgtctcgcctatgagcgacggtcaccacggtgacaaggatctgctcgtcgttgatcgagtacagggctcggtattcgccgcgacgggtgctgtgcagttcctcgaatggcgcatcgaggcgcttacccagacggtgcgggttgtcagcgaccggccccgtgaggtggtcgtgcatcgccatggccgcagccaaggggaggcggttcatctggcgcctggcggcagccgtgtagctgatcgtgtagctgcctcgtgggctcattcgttggccgcgcgtgctcggaggtcctctgccatctgatcagcggaaaccgttcggccggctgccatgtcggcgagtccttcgcggaggtcccgtacgagctcggagtcggagagcacttcgatcgtttctcgcagccgatcgatctcgcccgacggcacgacgtcggcaacgtgctcgccatgcgcgatgacgggcacgctctcgccacgttcggcacgctggacggcttcgttccatgacgccaggccatcggcggacacgctcactggctgctcgctcatggtgttcattgtgcggcatcctctcttcggtcgaccagcgccgaaggctggaacagggtttgtcagggagtgccctctaggagctagaccgctggtgaggccgtgaacgccgctctagcggcccctctagatctagaggccgtcacctctagatctagagggctccgtagcggccctcaggccgcctctgacgaaccgcgtgcgtcctgccgaggcctggtggggcgagtagcgccacagccgggagatcggcgctcaagaggccctgagatcgccacagacgccgcgaaggcgtccacccggtaccaggaggccgggcaaccgggttgtgggccgcgccgcgctccgcttcgtcggctcagttcccggctcagttcgcccccgtacaaccacccaaccactgttccagcaattgcgctgaccagcagttttgtacgactttgtgctgatgtgaaccacagaccggagatct

SEQ ID NO: 96 - DNA, 17501 bp, Saccharopolyspora erythraea
ccgcattcggacgccgccttgttggaaggtgcgcgcactggcaatcagtcgaacgtgcgttctatgctttgcgtgctcggtggtggggaagaccgccgggccccgcgggccggtcactgcaccccattggtggcgcggcccgccctttacatcggggcggggttggtgacgagcagagatgaccaatggtggggtacccgccggcctgtgggcgagcccaggcctgtgtgggtttacctgccgttggtcttcccgcctgcgcgggtggtcagcagggaggttccgattggtgtccgggcctacgggatcgacaacaccgccaccgtccctggtgagctgctggcgtggcagttgaccgccactggcgattggtgggctcagatccggctcaccttgcacaaccgcaaccagcgcggcgcgctggagaccgagctgtgggcgcccggagccgccgtacgcccgcggtagcaggtcaatgggtatcgcggtcgccgctgcgcccggcgcgggcgtgttcgacgctggcccgcaacgcctgcatgaggtcgacggcttcggtctcgcgtggcggtgggatgccgggttcgagagtgccgccggcgcgtttggcgtcgatgagtgcctggacctgttgctggtaggtgtcgtggtagtcggccgggtcccagtcgatggccatggcctcgatcaactgcacagccatccgcaactcctgctcggatgcaccgctgtggtcgggcaggctggcaccgagttcgcggtgggggtcgcggacttcgtcggcccagtgcagcaggtagaccgcgagcacctcgtgttcggccttgagcgcggcgaggtactggcggttgcgcatgacgaaggtggcgatccctgcccggttggcggtggccagggcttcccggagcaggccgtagaccttggtgtactgcggtccagatggggcgaggtagtaggtgtcgcggaagtagaccggggcgatctcgtcgaggtcgacgaacccgacgatctccagcgtccgggagcggcccggggcgatgtcctccagttccttcgggtcgacgatgacccagccgtcttcggtggggtagcccttgacgacctcctcgaaaggcacctcctggccggtgcgctcgttgacccgcttctgccgcacccggtcaccggtgccgcgctggacctgatggaagcgaatcgtgtgccgctcgaccgcggtgaacaactgcaccggcaccgacaccagaccgaaacgacagcactccaccccacaccgcagacgccatcagcaccacctccctccagccgacgtgaccgaccccagcaccaactgaccacgggaaccagaggcagaggtcaccccttggcaaaacccccagcccgtcgagaagtccgctctacggcgatctcagaggctcttgagaaagcatgtgcggtcgctcgtccagggccaccggataagggtcaccccgggtcttgggccctgtgtcgcagcctcagcggcgacgaggccacccttctgcgtcagctagccatgccgcgtgccgggctcctaagcctccgcgcgaggtagcttcgatgcatggagctggttccccgcgccgccacgatcgcagagcacttcgtccgcccacttggtcggcgatggctgcacgtccaagctgtcgccgaacgcgcgcatgaactgagccacgcagtgccggccgccgaccgggacatgctcgtggcggctgcctggctgcacgacattggatactcacccgagattggacacaccgggttccatccgctcgatggggcgcggtacctgcaagccgaggactggcccgaggtgctggttaacctcgtcgcgcaccattccggtgcgcggttcgaagcagcagagcgagggatggcaggtgagctagcagagttcccgttcgacgactccccgctgctcgacactcttgcgactgccgacctcacgacgggaccatccggcgaacggctgacctacgacgaacgcatggacgagatcctcagccggtactcacccgacgacccggtgtatcgcacgtggaccaaggcgaggccgatcatcgcggaagccatcgcgcgtaccgaccgccgccttgccggcagtcatccgatgtagggctcggtccgcgcgtcgtcgagagcgtggtcgatacggagccgcatagacgggtggatattgaggccgttgaggtccgctgggggaacccagcggacctctttggtttcactgccgtcctctcgcggcgtgccaccaatccatcggccctcgaagcacagagagaactgctgacggacctcaccgtcgtcataagccatgacatggtgcgggttggtgtacgtgccaacgagcctgacaacctcaatcgtcagcccggtttcctcctggacctcgcggaccacagtgtcggcgatgctctctccagcgtcatgtccgccgccgggtagagcccagaggtcgttatccactttgtggatcagcaagatctcgccgcgctcgttcctcaccgcagccgtcacagacggcaccaccgagttggcctccggcgcgttcggatcgttgaagtagtccacccttgccattcgtgcctcctacgtcggttcgggcgtggcctgctcccacacccggtcaaaggactccatgtagtgcttccacatccgaccaccaggcaactcgcgaaggtgcatgacggggttctgtccggcgagcgcgccgaaggcatgtccgttcacgagcaactgtccgtcgaaccggtagagcgagttgtagaggatcgttccgtgtgtccgcacatcgaccttgggcaggcccgacacctcgctcaggtaacggcgcatcatctgcacgcggccttcgagcccacccgtggtgccttcctccatggctcgttggacgactgcgggcgatttctcatccccgaccacgaagcggaactgaacgccttgttcggccttctgtcggacgatcgggatgatgttgtgctgctcgacaaggaactgccccgagaagacgaggacctccatcctctccttcacgccagcgatcagctcgttccacagcgagtacggcacgtccgaacgtgtcgggtagaggtggaccagctcggtctgcttggtcggctgcgtatggaggtcgcccgccaggtcaggccataggtgtacctcgtcgatgcttagcaaccccgcaagcttcttccgcgtcgatcggtgcggcttccggccgtcgtgggtgatccaccgctcgacggtcttgcggtcgacctgtagacgttctgaaacgtcctcgatcgtcagccccgcagcgttgaaagctgcacgtaggcggtcattcggcactctgcccgctcccctcgcgggacgttcccaggacggattgggacgttacccggtcgatcagaacgtcttcaagatccctgcaaaggccgtgggagcgtcccgcgaattgccgtgaactagagccatcccaggagaaaccaactccaacaaccggcgccctccagggcaacggcggagagagcttaaggagatggcgatggccaagagtccgattccgatgatgaagaccggcggcggtcttctaccgaagttcgttggcgcgctgctcacgctggcgttcctcgccatggtgatcaagcagcccgccgccgcggccgagatgctgaccggcgcgggcgcggcactcggcgcggcggtggaagggctcatgagcttcctgctgcagctcggaaagtagagagcgctcatggcgaagtcctgctacgtcgtgtcgctccccgaccgttcggcgagcattgccaggcgggcttcgaggtcggccagctcagcgctgacgcggtcgacctcgacccggaggccgacgacggcttggcgaacggcgttgatctcccctgcgacgtcgacgtcttccgtgcgatcacggacctgcgcgccaacgtgccgcttgatcgtcacgaggcccgcgtctcgcaccagcttcaccgcggcgatcgccgtgttcaccgccacgtcgtagtgatcggcgagctggcgatacggcggcagtgaagcgccaggagcgaactcgtcagcctcgatccgagcgatcaggtcatcggcgacacgtcggctagctgggcgtttgtcttcggactcagcgctcgcactcacgtgatcagcctacccaaaacgtcttgagacatccggttgacaaggccacccccaacccttaatgtctcaacacatcaaggatgtctcaagacatcagagcttaggagaacgacatggagtacctggacttcgagccgtcggcagatgagctggcgacctgcgaggcagagctggaggactgggagcggctggccgtacgggactactgggccgcggtcatcgccgaagagttgggagcccccgagccgcaggctcggccgtcggtgtggcggtcgatgggcccgggcgagcgggagcgcttgcgccgcaccgcgcggcgggcggccaacaccacgctgcgactggtggccgacaacgtcgcggtcgagcagggctcggcagctcccaacttcggggaggcggcgtgatggccggcctcgagcctgtcgacatcacggcgctgacacggacgtcgattccgctggaggcggtgaaccgcttcaacggcggaacggtgtggagcacgtggggacagttccacctcgctccgtgcgactgccacagcctctactggtctgaccgcgacgtcttctacgtccgcgacggggagttctacgcgtgcccgctgtgggggaccgagtgatggcgcggcagtggccgatcgtcaagacggtgctgtgggacggcacgtgggagttctgccacggccacctcgacgggctgccggtgttcgcctggcgcggtcgcccttgctcggtcccgtcccggctcgagctggccacccgccgccagctccggcagatgggcctgtgccccggtggggcggatccggtggcgctgctgcggttccgccaccgccagccctaccggcgcgaagagcttgccgagctgttccgcatcgacctggccaagcccaagcgcaccgccacccccgcgcagcgggaagcgatcgagcgggcgctgaccgcccgtcgtacgtgcccgacctgcccgcccgggcagcaggtcaagccctactacctgccgacctcggccgggcagtgctgggactgctacctgcccgacaccgccgcggcctgacgaaccacacagcaccaacacgacccatcgcatcaggagaacgagcgtcatgtccgagccccgcatccagatcggcggcaagacctacaccagcgagaactggcacgagatcgccgaggacccgaacgtcaccccggccaacgggcaggtcgtcaacgtcgcccacggcgacaaccacggcatccaggccggcgccatccacggcggcatcaccttcaaccgcaactgaccctcagggagagatttcgtgtccagcatcgacgacatccgcagcgtgctgtcgcaggccaacctccagtccggcgaggtgatggccaccctcgtcgagtcccagcgccacatcgaggccctggccaacatggtcgccaccgtcacccagggctcggacaacgagctggtgcagcgggccctggccaccttcggcctcgcccggacccagctctccgaggtcgtcggcgtcatccacgacggaaccgaccacctcagcaactaccagcagaccctctgaccccgttcccccagcacaccaaccccttcttcaggaggaagccgtcatggccggtatcgaagacgtccgcgcgaacctgtccgccgccaccacccaggccagcgaagcgctctacgcgctcaagcaggccgcgctgaccatcaacgaggtccagcgcgtgctcgacgacaccgtggccagcagcgcccgggagtcggcccagcacgccatcagcgcgttccaccaggcgttcagccaggccgaacaggcccaggagctggtgatctccggcagggactcgatcgacacctacgccgcccagctctaaccccacccgcacatccacccgctctgtcgagaacgcgaggagaacgaaccgcaatgtcgagcatcgaacaggtccgcgccgccatgcagtccgccacctaccgcaccgaacaggtcgtggccgcgctgcagtcctccgcgctggagctggaccagatcgacgccctgctgcagaacgtcggccagggcagcagcaacgagcactacaaccgcggcgcgctggcgctgatccaggccaccgaaccgctgcggcaggccctggagctggtgcgcaccggcgagaccgacctcaacacctacgccgcccagatctagccgcccgcctgcgagtccgaaagggaccaccaccgtgatccagctcgccattaccagcttcggctacctccacggccccgcgccggaggccaccgccgtcatcgacctgcgcaaccacctgcgcgacccccacgtcgaccccgccttccgccagctcaccggcttcgacctcgccgtgcacgacaaggtcctggccgcgcccggcgccgccaatatgcgcgtagccctggcggagttggccgccgccctgctccacaccgggagcgaaaagctcgtgaccatcgccctgggctgcgccggcggacgccaccgctccgtcgtgctggccaacgacctggccaacgtcatgcgtgtctgcggctggcagggcgaactcgaacaccgcgacatcgacaagcccgtcatccaccgcaccaccaagtgagaggagcacatccgatgagcctgaggagtcggaactgggaccgctcgcccgaaaccgagggtgaccgccgcttccacgacctgcgcgacagcggctacaccggcccgatcgaccaggacggaaaccccgtcaccagcggccgggacgccgacatcctccgccgcatggccgaggaacgcggcgaaaccgtcgactggtgaccccgccccggcccggcagccgcaaccaccacgaagcacctgccgggccgggctccccactgatccttcgaatctgcaaggagaagcccgatgctagaccccgagaccggcgacgtgacggtcacgatccccgctgacctcgcggccaagctgagcggcgcctacgaggcgtggcgcgccgccctcgacgacgtcgacgccagcggcgtcgacacggacccaacccatctggctgccctgtggcgggcccgctcgcgtaccgagctggctctcgccgatgtctacggcgagttggcctactccgtcggcccgttcgtgcccctgatcaatgcggtgtttcgcggcgtcgacgcctgccgaggcgccgccgaccgctacgtcgggatcgccgaacgcctcgaagggagggcgtcgtgaatcgcgtcttctcgatgctgcgagtcgcggtccgcttcacgggctgggtggcggtcccggtgctgctgctggtggcgctgtcgctgctgtccggcgtggtgcgcacgaccgccctgggcgccttctgtgtggtcgtggcgctcaaggccgcgatgaccgtgctcgacctggtcggcgagtacgccccgaacccccgttacggcttccgattggaggtgaccggtcgatgaccgcggcgaagagtgcctacccagtgccggtggacgagctgctgccgcaggcccgccagctcgccgacgacctgggcacgatcccgccccgcaaccggctgatgtccgagctgaagatcggggcaccgaaggccaacgcactgctggacaagctcaaggccgacccggacccggccacggcccgtccggcgggtctgcacctggtggctgagggccgggctctgcccgagtcgacgaccgagccggacccggtcagcgacgcccctgccgagcctgccgcaccgaccccgtcggttccgggggcggacccggccacggtcgaggcgcacaccccggcggcggaggggtcgtcggagcaggtcactgcgcccgcagacccgggcacgcccccgggcgaggtccgggagcgcaaggcggtgtcgacgtggccggtgctgctgctggcgctgccggcgttcgtggcgatctggtcgggctgggtgggcctgggcgggctcaccgggttcgggatcgtgcacccgcttccggggatctgggacgagttcgaactcaacaccgcgatcacgctgccgatcggggtggagacctacgccgcctacgccctgcgggtctggctctccgggcaggtcccggcgcgagcccgccacttcgcgaaggtctccgcgctgggctcgctggccctgggcgcgctcggacagatcgcctaccacctgatgaccgcggcaggcatgaccgccgccccgtggtggatcaccaccatcgtcgcctgccttcccgtggccgtgctcggcatgggcgccgccctgacccacctgctgcacacccccgacctggaggtgacccgatgaccacccacgaccacgagccccagcacacccccgaggtcaccgaacccgacaccgaggcgcaggtgttccacctgcccgtcgaacgcgccgagcccgccgacaccgcaggtgccggtgagggtgagggtgaggtgatcgagggcgagatcgtcgagccgccccaggttgaccagcccgagccccgcggcaccgggtccgcgctggcgcggacggagaagcgcgagccgatccttcccagttgggccaaggactcccaggagttcgccgacaccgcacggtgggcgctgggctatgccggacataccgccgggtttcacgcggtgcgctgcccggtctacatcgcccgcatcatcgcccgggtgccgcagggcacgctgcggctgctgcgcgggctggctcgatggttgaccgacgccgaaggccgcccggtgcgcaacgccgcggcccgtcgcgaggacgccacggagtacctgaagctgtccacgcagcgcgacgggcggatccgctcccgcgcggtcctcacggccgggctgggggcggccggcatcgcggcgttcctgctcggccgggcgatgctgcccgagctggtgcagtggtcgatcgtggccgccgcgatcggtgggctgggctggctcggtgctccggcggacaagccgatcgccagccgggcgatcgaggccacccgggtgcccaagctgaccagcgacgcggtgacccgcgcgctggctgcgctggggatttcccagatcaaccaggccatgggcaagggtggggagggcatcggctacccgcggccgatcagccgcgatggcaagggctggcgcgccgacatcgacctgccccacggcgtgaccccgggcgacatcatggaccgccgcgaaaagctcgcgtccggtctgcggcgcccgaccggctgcgtgtggcccgagtccgacaacgccgagcacgccggtcggctggtgctgtgggtcggtgaccaggacatgcgcaaggccaaacagtccgcgtgggcgctgcgcaagggcgtgcaggtcgacctgttccagccccagccgttcggcaccgaccagcgcggccggtgggtcgacctgcggctgatgttcaccagcgtcgcgatcggcgcgattccccgcatgggcaagaccttcgccctgcgcgagctgctgctcatcgccgccctggacccgcgcgccgagctgcacacctacgacctcaagggcaccggcgacctcgacccgctggagaaggtcacccacgcccacggtgtcggcgatgacgacctggacctgcacctggccgacatgcgggccgtgcgcaccgagctgcggcgacgggccaagctcatccggcagttggccaagcagggcctcgccccggagaacaaggtcaccccggagctggcctccacgaagtcgctgggcctgcacccgatcgtcatcggcgtggatgaatgccaggtgtggttcgagcacgccaagtacggcgaggagttcgaagagatctgcactgacctggtgaaacggggtccggcgctgggaatcatcatcatgctggccacccagcgcccggacgccaagagcctgcccaccggcatctcggcgaacgtctccacccggttctgcctgaaggtgcagggccagaccgaaaacgacatggtgctgggcacctcgaagtacaagcagggcgtgcgggccaccacgttcgcctgggccgacaagggcatcggttacctggtcggcgagggctccgacgcccagatcgtgcgcaccgtcgccggactcgacggccccgccgccgaaaaggtcgccgcctacgcccgccacctgcgcgagcaggccggaaccctgtccggacacgccatcggcgagaccgtcacgtccgatgaggaccaccgccgggacacgctgctggacgacatcctcgccgtgaccccggaaaccgaggcgaaggtgtggaacgagaccaccgtggcccggctcgccgagctgcgccccgaggtctacgggagctgggaagccgaccagctctccgccgcgctcaagccccacggcatccgagccaaccgtcaggtgtggggcaccgacgagagcggcgagggccgcaaccgccgcggcttccaccgcgacgacatcaccaaaaccgtcactgaacgtgaccgaggacgggaagcgagctagcccccgccgggtcgctaggtctagcggccggccccgctagacctagcggctccgctagcaacccatctccaccgccaccagcgccctagtccctagcggcgcctggccaaacacgcccaaaaacgcctgctggaggcacctttgacccccgccgacctcgctaacaccagcctgaccctgctcctcggaatggtcacgctctgttactcgctggtgtgtgtgatctggccgttcaaggactgccgcacctgccgcggcaccggccgcctccgctccccgttcctgcgcagctaccgcctctgccccgcctgcgaagccaccggcctgcggctgcgcaccggacgcaaagccgtcaacgccctgcgccgcgtccaccgccgcaaccgcggccactgaaccggcaaccggaaggacaccaccgtgatgcacatcgtcggcagcgcccgctccgccctgcaccagctcggactcaccagcgccaagaccctctgcggcaaaaagctccgccagccagaggacggcgccgcgaagaacgccccgctgtgcccggagtgctaccgccgctccggttggacccacgacaagcgccggcagtgaacaccctccagaaaggacacatcgccatgagcaggcatcgcctcgaaccccgcaaccccaacaacgttcgcgaggtggtcgtcggctgggactcgcccatgcggaccttctacgccgtcgtcgaagaccacagcggcgccatcccggtcgacctcggcgactccatcgagcccgtgctccgtcccggggccgtgctcgatgcggtccgcccctacgccgccatcccgcccggcctcgacgacgagctgctgcgcgacgcgctggccgaccccggcatccgcgccgcctgacccgaaccccgtccacgcggccccgcccgccattccgccacagaacccgggcggggccaccacccctcaaagataggtaaccgtcatgttcgcagagatcgccatccccgtccttaccggcggatgggccgccgtcgccaccgccaccaccatcaactaccgacgccgcacgctgaccgacccggtcaccggcatcggcaaccgcgccgccctctaccgcaccgcccgccgcaccaccgcccgcagcgggctcgtcggactgctcatggtcgacctcgaccggttcaagcagatcaacgacacccacggccaccccttcggcaaccgcgtcctgaccgccatcgccacccgcctgattgagaacacgctgcgcggggagcgcgcggtgcggctgcacggcgacgagttcgcgatctggctcggacgcatcacctccaccgcccgcgccgaaggccgcgccctccagatcgccgacgccctcgccgaacccctccagatcggcggccgtcggctcgtggtgcccggcagcgtcggcgtcgccgtcgcccccgcccgcaccccactcggcgaactgctgaacaccgccgaccagcacatgtaccaggtcaaggccacccaccacctgcccgcactgcccgccggcgagccgcggcgcgcccgcgaccgggccaccccgcccgaccacgccgcctgaaacccgaccacacccacgacaggaggtgactccgcgatgaccgacccgatccggctcgcgcactggctgatcgagcacggcatgtacgtcttcccgctgcgcccctactccaagcgcccgttcggcaactgccgccgctgcaaggacaaccgctgcacccagccgtgcccgtgcctgaccgccgaccgtccctgccacggctacctcgccgccaccaaccagcaccgccgggcccgccgctggttcacccgtatgccggcagccaacgtcggcatcagcaccgacctctccgacacggtcgtgctcgacctcgaccgcaagcccaaggctcccgcagccgccgcgcacgacgtgcccatcctcgtcgccgacggattgggagccctcgacgcaatcaccacgcacgagggcgccgactggcccgacaccctgaccattgccaccccgtccgaagggcgacacctgtacttccgccgccctgcggggttggaggtcgccagcgacgccaacggccgggtcgggcaccagatcgacatccgcgcccagggtggctacgtcgtcgcccccggctgccagatcaccgcaccacccgaagacgtcttcggcacctacacccgcgtatcgaccacagtggacatcgcaccgctgccggactggctgcgccctcgggtcaccccgccacccgcgacaccgaccggaccggggaaggctcccaacctcgggcgaatccgtcacggggacggtcatgagcccggttactggaaaacggtgtggaagagcgtgctcgacaaggtcgagtacgaggacggcgagcgctggaagttggtctacaacgccgcccgccgcctggccaacctcgccgtgcacgacggcgccccctggaccgagcacgaggtgctcgacgagctggaggccgccgcgatccgccgccgcgagcacaccggcaaacccaccgagcccgccaccgcacgccgcaacgcccagcgcggctgggaccgcggcacccacgacggccccgactccctgatcggcctgggcggcgcggcatgagcccgacatcccggaaggaatccctcgtgaccagcccacggcttctggacctgttctgcggcgccggcggcgccggcaagggctacgccgacgccggattcgacgtcgtcggcgtcgacatcgccccccagcccgactacccgttcgagttccaccaggccgacgcgctgaccttcctcgccgcccacggcaccgagttcgacgtcgtgcacgcctcgcctccgtgccaggcgtccagcgcgttgaccaagggcaccaaccgcggccggtcttacccccaattgatcccccagacccgcaccgccctggtgcagctcggggtgccgtgggtgatcgagaacgtcgccggcgccccgatccgcaaggacctcatgctctgcggcgagatgttcggcctcgccgtcctgcggcaccggttcttcgagctgggcggctggaccacgccgagacccccgcaccccgcccaccgtggccgggtctccggaatgcgccacggccagtggttcaccggcccctacttcgccgtctacggcgacggcggcggcaaaggcaccgtcgcccagtggcagcaggccatgggcatcacctggaccgacgtccgcaagtccctcgccgaagccatcccgcccgcctacacccaccacctcggcaccgcactactggccgcccgcgccgccagccctgcggcgacggccgcatgaccacccgatccgcacccgatggaggtgagcacgtgaccacacccgctcagcagcccagcgacggcgccgtcctgctcgacgaactgcacgccaccctgaccaaattcgtgatcctgcccagcccgcaggccatcgacgccgtggtgctctggatcgccgccacccacgcccaacccgcctgggcccacgccccccgcctggtcatccgcgcccccgagaagcgctgcggcaagtcccgactgctcgacatcgtcgaaggcacctgccacgagccgttcctgaccgtcaacgcctccccctccgccgtgtaccggtcgatcagcgacgacccgcccaccatgctcgtcgacgaggccgacaccatcttcggccccgacgccggcaccaacgaagaagtccgcgggctgctcaacgccggacaccagcgcaaccgacccgccaagcgctacgacgccgcatccggccgcgtcgagtccatccccaccttcgccatggccgcactagccggaatcggcgccatgcccgacaccatcgaggaccgcgccgtcatcgtgcgcatgcggcgccgcgcccccggcgaaaccgtcgcgccctaccggcaccgccgcgaccgcccacacctgaccgcgctcgcgaagcggctcgccgcctggctccgcgcctcgatgcccgacctcgaacgcgccgaacccgacatgccgctggaggaccgggccgccgacacctgggaaccgctcatcatcgtcgccgaccacgccggcggcgactggcctacccgagcccgcaacgccgcggtcgacctgctggccgaagccgccgacaacgaccaaggctccctgcggacccggctgctcgtcgactgccgcaccgcattcggtgaccaccccacgctgtccaccaccgaactgctgcgccagctcaactccgaccccgaagcaccctggcccacctacggcaagaccggactcaacgccgccaagctctccaagctgctcgccgaattcgacatccgctccgccaacgtccgcttccccgacggcacccaggccaagggctaccagcgagcccacttcttcgacgcctggacccgctactgccccgacgccccgcacgaccggccagagggggtgccgtcccagccgtcccaagcgtcccaccgcaggtcagagcgggacggcttgaccctctgggacggcatcagccgtcccaacgacgaacccgacccagacctctgggacggcacaagccgtcccaccgcaccgagccgtcccagcctgacctgcattgggacggctgggacggctgggacggacacccctcccagcactaacaccaagggggccgcatgaccacgaacgtcacccaactcgccgccacgctcgcctcgctcgcggccctgctcgccgaacagcagcccgccccggaacccgagcccgaaccggccgcccgcaggctgcccaaccgcgtgctgctcacggtcgaggaagcggccaagcaactggggctcggcaggaccaagacctacgcgctggtggcgtctggcgagatcgaatctgtccggatcggtcggctcaggcgcatcccgcgcaccgccatcgacgactacgccgcccgactcatcgcccagcagagcgccgcctgaagggaaccactatggaacaaaagcgcacccgaaaccccaacggtcgatcgacgatctacctcgggaacgacggctactggcacggccgcgtcaccatgggcatcggcgacgacggcaagcctgaccggcgccacgtcaagcgcaaggacaaggacgaagttgtcgaggaggtcggcaagctcgaacgggagcgggactccggcaacgtccgcaagaagggccagccgtggacagtcgagcggtggctgacgcactgggtggagagcatcgcgccgctgacctgccggtacaagaccatgcggggctaccagacggccgtgtacaagcacctcatccccggtttgggcgcgcacaggctcgatcggatccagaaccatccggagtacttcgagaagttctacctgcgaatgatcgagtcgggactgaagccggcgacggctcaccaggtacaccgcacggcgcgaacggctttcggcgaggcgtacaagcggggacgcatccagaggaacccggtttcgatcgcaaaggcacctcgggtggaagaggaggaggtcgaaccgcttgaggtcgaggacatgcagctggtcatcaaggccgccctggaacgccgaaacggcgtccgctacgtcatcgcactggctctcggaactcggcagggcgaatcgctcgcgctgaagtggccgcggctgaaccggcagaagcgcacgctgcggatcaccaaggcactccaacgtcagacgtggaagcacgggtgctctgacccgcatcggtgcggcgcgacctaccacaagaccgagccgtgcaaggcggcctgcaagcggcacacgcgagcttgtccgccgccatgcccgccagcttgcaccgaacacgcccggtggtgcccgcagcgaaccggtggcgggctggtcgaggtcgacgtcaagtcgagggctggacgacggaccgtgacgctgcccgaccaactgttcgacttgatcctcaagcacgaaaagcttcagggggccgaacgggagctcgcgggcacggagtggcacgacggcgagtggatgttcacccagcccaacggcaagccgatcgatccacgtcaggacctcgacgagtggaaagcaatccttgttgaagccggagtccgcgaggcgcggctacatgacgcacggcacaccgccgcgactgtgctgttggtcctcggagtgcccgaccgggtcgtgatggagctgatgggctggtcgtccgtcaccatgaagcagcggtacatgcacgtcatcgactccgtccggaacgacgtagcggaccgcctgaacacctacttctggggcaccaactgagacccagactgagacccaaaacgcccccgtcgagatcgacgggggcgttttggcagctcttggtggtggccaggggcggggtcgaaccgccgaccttccgcttttcaggcggacgctcgtaccaactgagctacctggccgttcgcgcccggctcaaagccgaaccgctgtggcgacccagacgggactcgaacccgcgacctccgccgtgacagggcggcgcgctaaccaactgcgccactgggccatgttctgttgttgcgtacccccaacgggattcgaacccgcgctaccgccttgaaagggcggcgtcctaggccgctagacgatgggggcttggccgattcggaaccgacccggcctcgcctccaaccggctttccctttcggggcgccccgttgggagcagtgaaagcttacgacacaccccccagcgccccacaacgggggggtccccaaacctcacgagcccccgcgcggcccacgcccgccggtcacgtcggtcgccaccatatgccatctgaccagccttttccatcgcctatcctcagtcggcccact

SEQ ID NO: 97 - DNA, 12 bp, Saccharopolyspora spinosa
aggaggtcccat

SEQ ID NO: 98 - DNA, 25 bp, Saccharopolyspora spinosa
ccaggaatcggaggggcagtaccga

SEQ ID NO: 99 - DNA, 25 bp, Saccharopolyspora spinosa
gcaacttcctggagggaaacgccac

SEQ ID NO: 100 - DNA, 25 bp, Saccharopolyspora spinosa
tcgtcacggcagtgagggattgggc

SEQ ID NO: 101 - DNA, 25 bp, Saccharopolyspora spinosa
cgaaatcccggcgaggaagggcgcg

SEQ ID NO: 102 - DNA, 28 bp, Saccharopolyspora spinosa
cgcctcggcccccttcaggaggagacag

SEQ ID NO: 103 - DNA, 26 bp, Saccharopolyspora spinosa
ctccagacgcccacgcaaggagaccc

SEQ ID NO: 104 - DNA, 19 bp, Saccharopolyspora spinosa
actagtaaggaggtccaac

SEQ ID NO: 105 - DNA, 15 bp, Saccharopolyspora spinosa
aagaggtatatatta

SEQ ID NO: 106 - DNA, 20 bp, Saccharopolyspora spinosa
ccaccgctggaggtatccgg

SEQ ID NO: 107 - DNA, 13 bp, Saccharopolyspora spinosa
aggagagatcggc

SEQ ID NO: 108 - DNA, 12 bp, Saccharopolyspora spinosa
aaagaggagaaa

SEQ ID NO: 109 - DNA, 15 bp, Saccharopolyspora spinosa
attaaagaggagaaa

SEQ ID NO: 110 - DNA, 19 bp, Saccharopolyspora spinosa
agaaggtggaggtcacacc

SEQ ID NO: 111 - DNA, 16 bp, Saccharopolyspora spinosa
aagggctgttggaatc

SEQ ID NO: 112 - DNA, 18 bp, Saccharopolyspora spinosa
attgaggtcgagggtcgg

SEQ ID NO: 113 - DNA, 22 bp, Saccharopolyspora spinosa
ggcggtgaatgatccgccgcgc

SEQ ID NO: 114 - DNA, 20 bp, Saccharopolyspora spinosa
gacgaggaagaggcgccaca

SEQ ID NO: 115 - DNA, 16 bp, Saccharopolyspora spinosa
acgttacgctcgtcgc

SEQ ID NO: 116 - DNA, 19 bp, Saccharopolyspora spinosa
gggacgttacgctcgtcgc

SEQ ID NO: 117 - DNA, 21 bp, Saccharopolyspora spinosa
tcgtgacctcggtgctgaaca

SEQ ID NO: 118 - DNA, 15 bp, Saccharopolyspora spinosa
aggaggaacaatcca

SEQ ID NO: 119 - DNA, 18 bp, Saccharopolyspora spinosa
ccgcaggaagtgagtgac

SEQ ID NO: 120 - DNA, 20 bp, Saccharopolyspora spinosa
cgtgacctcggtgctgaaca

SEQ ID NO: 121 - DNA, 18 bp, Saccharopolyspora spinosa
aattcccggggatctacc

SEQ ID NO: 122 - DNA, 17 bp, Saccharopolyspora spinosa
cgaggcgaacgccagcc

SEQ ID NO: 123 - DNA, 16 bp, Saccharopolyspora spinosa
gcgaaggagagccccc

SEQ ID NO: 124 - DNA, 17 bp, Saccharopolyspora spinosa
ccgaaaggaacgccgac

SEQ ID NO: 125 - DNA, 17 bp, Saccharopolyspora spinosa
gaggaaaggaaaacgaa

SEQ ID NO: 126 - DNA, 18 bp, Saccharopolyspora spinosa
gaacggaagggacgcctg

SEQ ID NO: 127 - DNA, 21 bp, Saccharopolyspora spinosa
cggcgggtcggagaggagtgc

SEQ ID NO: 128 - DNA, 1048 bp, Artificial Sequence
  transposon mutagenesis payload sequence LoF
ctgtctcttatacacatctccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatcgatagccgccccgcagggcgctccgcaggccgcttccggaccactccggaagcggccgtgcggtcggaggtaccagatgtgtataagagacag

SEQ ID NO: 129 - DNA, 1352 bp, Artificial Sequence
  transposon mutagenesis payload sequence Gain of Function - promoter
ctgtctcttatacacatctccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatcgatagccgccccgcagggcgctccgcaggccgcttccggaccactccggaagcggccgtgcggtcggaggtaccggtaccagcccgacccgagcacgcgccggcacgcctggtcgatgtcggaccggagttcgaggtacgcggcttgcaggtccaggaaggggacgtccatgcgagtgtccgttcgagtggcggcttgcgcccgatgctagtcgcggttgatcggcgatcgcaggtgcacgcggtcgatcttgacggctggcgagaggtgcggggaggatctgaccgacgcggtccacacgtggcaccgcgatgctgttgtgggcacaatcgtgccggttggtaggatccccacccaacgcaccccaggaggtcccatagatgtgtataagagacag

SEQ ID NO: 130 - DNA, 3068 bp, Artificial Sequence
  transposon mutagenesis payload sequence Gain of Function - counterselection
ctgtctcttatacacatctggtaccagcccgacccgagcacgcgccggcacgcctggtcgatgtcggaccggagttcgaggtacgcggcttgcaggtccaggaaggggacgtccatgcgagtgtccgttcgagtggcggcttgcgcccgatgctagtcgcggttgatcggcgatcgcaggtgcacgcggtcgatcttgacggctggcgagaggtgcggggaggatctgaccgacgcggtccacacgtggcaccgcgatgctgttgtgggcacaatcgtgccggttggtaggatccccacccaacgcaccccaggaggtcccataagaggtatatattaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgcgcggccggacccgcacacacccgctccagacgcccacgcaaggagacccatgaacatcaagaagttcgccaagcgggcgaccgtcctgaccttcaccaccgccctgctcgcgggcggggccacccaggccttcgccaaggagaacacccagaagccctacaaggagacgtacggggtgtcgcacatcacccgccacgacatgctccagatccccaagcagcagcagagcgagaagtaccaggtcccgcagttcgaccagtccaccatcaagaacatcgaatcggccaagggcctcgacgtgtgggactcctggcccctgcagaacgccgacggcaccgtggccgagtacaacgggtaccacgtggtgttcgccctggcgggctcccccaaggacgccgacgacacctcgatctacatgttctaccagaaggtcggcgacaacagcatcgactcctggaagaacgcgggccgcgtcttcaaggacagcgacaagttcgacgcgaacgacgagatcctgaaggagcagacccaggagtggtccggctccgccaccttcacgtccgacggcaagatccggctcttctacacggacttctccggcacgcactacgggaagcagagcctcaccacggcgcaggtcaacgtgtcgaagtccgacgacaccctcaagatcaacggcgtggaggaccacaagacgatcttcgacggcgacggcaagacctaccagaacgtgcagcagttcatcgacgagggcaactacacgtcgggcgacaaccacacgctgcgcgacccccactacgtggaggacaaggggcacaagtacctggtcttcgaggccaacaccggcaccgacaacggctaccagggcgaggaatccctgttcaacaaggcgtactacggcggcagcacgaacttcttccgcaaggagagccagaagctccagcagtcggccaagaagcgggacgccgagctcgccaacggcgcgctgggcatggtggagctgaacgacgactacacgctgaagaaggtcatgaagccgctcatcacctccaacaccgtgacggacgagatcgagcgggcgaacgtcttcaagatgaacggcaagtggtacctgttcaccgactcccgcggctccaagatgaccatcgacggcatcaactcgaacgacatctacatgctgggttacgtctccaacagcctgaccgggccgtacaagccgctcaacaagaccggcctggtgctccagatgggcctggacccgaacgacgtcaccttcacctactcccacttcgcggtgccccaggcgaagggcaacaacgtggtcatcacctcgtacatgacgaaccggggcttcttcgaggacaagaaggccaccttcgccccctccttcctgatgaacatcaagggcaagaagacctccgtggtgaagaacagcatcctggagcagggccagctcaccgtcaacaactgaggtaccagcccgacccgagcacgcgccggcacgcctggtcgatgtcggaccggagttcgaggtacgcggcttgcaggtccaggaaggggacgtccatgcgagtgtccgttcgagtggcggcttgcgcccgatgctagtcgcggttgatcggcgatcgcaggtgcacgcggtcgatcttgacggctggcgagaggtgcggggaggatctgaccgacgcggtccacacgtggcaccgcgatgctgttgtgggcacaatcgtgccggttggtaggatccccacccaacgcaccccaggaggtcccatagatgtgtataagagacag

SEQ ID NO: 131 - DNA, 1716 bp, Artificial Sequence
  transposon mutagenesis payload sequence Gain of Function - solubility tag
ctgtctcttatacacatctccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatcgatagccgccccgcagggcgctccgcaggccgcttccggaccactccggaagcggccgtgcggtcggaggtaccatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccagatgtgtataagagacag

SEQ ID NO: 132 - DNA, 723 bp, Saccharopolyspora spinosa
atgaccacgttgagcctgcacggggcgacaacgctgctgtacgccgcgccggtctcgaccgagctgctgtcccagctgccgttggacaacctggccgcctacgtcgccacgatggccgccgacctggcggccagggaccgggaacggctggagcagggattggcggcggcggtcgagcgcggcgggccgtggttcgagcgtgaccgctacgagctggcccggtccctcgcgagggccgtccaggtcgagcccgaggcgtccgggtcgagctgagcccggaccccggattcgaaggtttcccggccggtggtgccagtcccgccgccccgtgtcgcacggggtttgaacaccgccaccggccgggttcccaaccgatcagcggaccgtttcggaaccgccgggaacagcacgaaccagctcccaaccccggggttgggagcaatcccggaaccaggttcccggccgccgggaagaagggttcttcaccccttctcacctgcatgggaagaaccccgagaagcggcaccggagaagcaccgagaacccgccgagaacccctctcccgaccgagccgatcgaccgggcgaccgagccgaccagccgggccgccgcagccgaccgggcgaccgagccgaccagccgggccgccgcagccgaccgggcgaccgagccgaccagccgggccgccgcagccgaccgggcgaccgagccggtgacctgcat

SEQ ID NO: 133 - DNA, 1259 bp, Saccharopolyspora spinosa
tcacagcttcctgaagtgctcgatgagccgggagtagttggcgttggcgcggcccgcctcgatcgccctggagaccacggcgacggtgtagcccgggagttcgtcgtcgatgccccgcgcccggctctcgctgactaggtcctcagccgacgacaggtggtgttgcagcgccccgaattccaccgggtactcgccgcgatcgacctcgtcgggccgggacaccggcatcggccgagatcagtccgccccggccgccgatgagttcgagcgtcgcacggtagcggtcgtgggccgactcggcgccgccgaagaacaccacgtcgtccgggtgcccgattacctgagcgatcgtcatgatctgcccgtgcaggtactccgcgcccgctgaggtgggccattccgcgaccgctcgggcctcggccgaggtgccatccgtcaggttgaccacggtgtggccggcgagcgcgtcgcccgccgattccaggagttcgcgcgcggtgctgctgccctacaccgtgacgacgacgagcgggctggccgcgacggcgtcatgaacggtcgcggcccgctgcgccccttggcgaccaggtcgtccgccttcccgctcgtgcggttccacaccgcggtcagtgtccggcggccaggaacgcgctcgcgagcgcgaagcccatctcgcccaaaccgatcacggtcaccgccggtcggctatcgatgttgctggtcatggctcatgccctcgctcgccccaagatgatcgagagcacgctagggacttcgagcgaacgcgaggtcaagacccctgttcagcccggtggcgtcaggcccagggacttgatcaggtcgtggatctcggcccggtagagcttgaccggttccctggtctgggggctcaggcggccgtaggtttccagcgaaaccggcccgtgcatgcgacgccagacgcgccggccgccgtcgcgggctcactgcggcagttcggggaacgactcgcggacctcgtaggccaggcgcgcttcgaagtccgatcagtcgtggtcgccgtcggcctggcgggtgcccgcctgcggccaggcagcggccaccaggccggtgcggccagcgcaagcccggtgcccggcctcggcggcgagcccgccgccgggcggtgcctggtagccggggactggggcgccatagatccgcaaccccacaatccggcgcaccacgtgcacgggcgtgcgcgtcgggctgtgatgcggtcgtgagctgcgatcggccat

SEQ ID NO: 134 - DNA, 1687 bp, Saccharopolyspora spinosa
ctaccgcgcgaatgcgggatcgccgtccagcacgcgaatcgccgcacttgcttgcagtgcaacgacattcagcacatggccgaccgagtcgtgtagttcggcggcgatccggtttcgctcggacaacgcctcgacctgccaccgtaaccactcgtgatcggacggtcccagcagcgtccgggcgagccgcgccaacaccgcgccgacaccctcgaccaggtggaaacccgcgacgatcaagcccaggccgaccagcggcgcccatcaatgcccgatgccgaaagcccggaacggcgagatgccgacgagctcgggcggcagcgtcagcgccggatcggcgagcagcagcaggatgaacggcggaacgatcagcgtcagcaggccgatgccggagccgagcgcgaggtggacgacgagccagcagacggtccgcaaccgatccggccgggtctcggcggcgccggattccggcagcccgagcagggattccgccgtggcatccgacagcgaccgcgtcgccggcagcaacccggtggccaccccggcgagcacgacgaacgcgccttgcgcgagaccgaccgcgacaggccgcaggccgagcccgatgtccgggcccagcagcgccccgagcaccagcccggacaccgccaggtacgccagcggccctgaggatcaggtgcacccatcgccggtacgtggagcgcggtgaagggtctgatcaggcgggacggcatggccacatcgtctctgcggaaccggcgcgcgatcgcaggttccgcacggcttgcggcgcgtcgcccgctggtacaggtagaccgacgacccgagcaccagctcccagcacatccagctcacgtagaacagcgcgccgggccagtagcgcatcccggcgatcgtgttcgacctgccggagatcaggttggggaggaccgtggcgagctggccgggcccccatgcatggatgaaaagcagcgcggcgccgaaccagccgccggcgagcagcagcccgcgcggcgtcggacggccagccaggccgggaacccagcgcggccagatccgcccgcaccggtggacgacggccagttacagcacgatgccgatcagcgcggcgggcacgaccagccaaccgctggcgaagccgtcccgcgccgggtcggggtagtccagtccgatggtgccgcccagcgcccagttggtcttgatcatcagatagggcagcgcgaacacgaggccgagatatccgagccagcgcgccggcggcccggccggcccgccgcagcgttcgcaggcccggtgatctcccggacccgcaggactgtcccggacccgcaggactgtcgcctggaacagcaggatcgcgctcagcaacgcgaaggcgcgggtggcgaacatcggccagccaggggaaagcccatcaggccggacacccgcgacagtcgaacaggatgccgccggtggcccagagcagcagcaggacggccgcgccgttgatcatgatgagcatgcggcgaggtcgcaacggcgacatcagcacggcggtgccgaccacgccgaccgctgcggccagtggtgtcgcccagcccgggagctcgaagaggacgaatccgcgcggcaggacgtgccgcgattcgacggagaggaaggtcagggtcggcgcgatcacggccgcccgactcggcttgagcat

SEQ ID NO: 135 - DNA, 1286 bp, Saccharopolyspora spinosa
gtgatcaaccgggacaccgcgtacttctgggagggcgcggcggccggtgagctgcgcatccagcgctgcggccggtgcgggctgctgcggcacccgccggggccgatgtgcccggaatgcggcgccgcgacgcggacgcacctggtctccgaagggctcggcgaggtctacagctccatcgtccaccaccacccgctgatgccgggcaaggacctaccgctggtcgtggcgctggtcgagctggacgaaggcgtgcgggtactgggcgagctgctcggagtggccccggaggacgtccggatcggccaccgggtggcggtcgacttccagcggatcgacgacgaactggtgctgccgggatggaggctccatgagtgacggaataccggtggcgctggctcggacgcggtcgctcgccgagctgaccatcggcgatgcgctgccggaggagcgcatcgaggtatcgccgaccttcgtagcacgtcagcggcacggttcgggcggcgttgccggaggtcgcgcggtgagcacgatttcgggcagcgcggcgatcgtcgggatcggtgcgacggagttcttcaagggatccgggcgcagcgaaccgcaactggccgccgaggccgttcgggccgccctcgcggacgccgggctggaaccgtccgatgtggatggtctggtgaccttcaacatggacaacaacaccgaaaccgccgtcgtcagggaactgggcatcccgagctgaccttcttcagccgcatccactacggcgacggcgcggcctgcgcgactgtgcagcagaccgccacatggctgtcgccaccggcacggccgacgtcgtggtctgctagcgggcgttcaacgagcggtccgggcgccggttcgggcaggtgcaggcggcggcagcaggcacgccgacctcggcggggctggacaacagctggtcctacccggtcggtggccacgcccggcacgcaggtcgcgatgttcgcccgccgctacctgcacacctacggcgcgaccagcgaagacttcggccgcgtcgcggtggccgaccgaaagtgcgccgcgaccagtcccaacgcctggttctaccagcggccgaccaccctggccgatcaccaggcgtcgcgctgggtcaccgagccgctgcggttgctggactgctgccaggaaagcgacggcggggtggcgatcgtggtgacgagccgggaccgggccgcggacctgcggcactcgccggccgtggtggcggcggcccagggcagcggcgccgaccagttcaccat

SEQ ID NO: 136 - DNA, 1864 bp, Saccharopolyspora spinosa
tcaatgcgacgttaccgggcgggatggcggattcacacctgtttcaagggttttccccgcggccccggctacctcggtcggccgggtacgccggggcgggtttgccacgggtgcgggccccgagcgggcggtactcgacgccgaaggcatccagccggggcaggtggtggtcgaccaggcggcgccggacgtccgcgaagccgcggtcgcggctgctccagggccaagatcccgcatccggtcgtgctcggctccggtcgccgcccggtcgtcgtagccgacgcacaacaaccgcagggtgccctggggcacgacgtaggcggcgttggcggcgatgccggtgtcgagccggtcgaggtattccgcgaccgctgcgccagttccagtcgaaccccggcggatcatcgttccagcccgcgatctgggtgcgcacggtgaccagcgtggcgtcggttaccgggcgtacgacaacccgtcctgtccgatgacctccagcgtcacgccctgcgacaccttcgccgtgtggtcgggtgcggcaaggatctgcaggtcggagtgtgcgtgcatgtcgatgaaaccgggcgcgagcactaggccgtcggcgtcgatcgcgcgttttccgctcaactgtgcgggttcggcgatctcggcgatccgcccgcgctcgatcccgacgtcggcgcggtagccaggcccgccgtctccgtcgacgacgctcgccccgtggaccacgatctccatgccggaacctccccgtgctaggagttcttgcgcagcggtcgtcccaattagaagaacgtgcgctccaggtcgatcacggtgtcgtcgtcgatgaccgggatcaggggtcacttgtcgaaaaccgtgcagggatgggagaggccaaagccgatccagtcgccgatctgcggatcggtgtaccgcatggtcaggagcgtgtgctggtcggccagcgacgtcacctcgcacgaaccggcggccagctcccggatctcgccctccgcgccgcggatcacctgcggttccggcaggtcttggtcgaacgacacgtcccggcggcccacggtcagcagcgccaggccccgctcaggccgggaggtgacctgcgcccacacccgcatcgcggggcggaacggcgattccgcgcccgccagccggtgctcgcggccgaacggcgagcggacccggtagaaaccgtcgtcgtgcagcaggtatccgccgctgcgcagcaccggcacgaccggcagcccggccggccacggctcggtgaacgcctcggcgacctggtcgaagtacgcgctgccgccgcaggtgacgatcacctcgtccaggcccgagaagtggcccgcctgggccagttccgccacgaggtcgcgcaaccgccccaggtatctgtccacagcggacagcgactgctcgctgacgtcgtgccccagcgcgccctcgtagccgccgacccgaccagtcgcagcatcgggctttccagcaccaggtcggcgatctcgcgcgcctcggcgatcgtgcgcgcaccgggccgggcgccgtcgctgccgagctcgaccagcacgtcgacctgccgggacgcgccgtggtgggccagtgcctcggccatcagctgcgctccgcgcaccgagtccacgaagcagctgaactcgaattcggggttggcgtccagttcgtcggccagccaggccagcccgctggggtccagcagttggttggcgagcatgatccgctcgacgccgaaggcgcggtaaacccgcagctggctgacgttcgccgcggtgatgccccacgaaccggtagccggtattgcgtaaaatgcaacatcggttgcgtagaatgccggattggtgtgtaccat

SEQ ID NO: 137 - DNA, 1212 bp, Saccharopolyspora spinosa
ctagcccgcttccacgatgtcgatcgtgcgggcgagcacgtggtgcgggtgcggctggtgggcgttgccccattccacggccgccgaatgcccggcggtcgcgacgatcagcccgacggcccggtcctgccgtggtccgccgaggccgccgtcttcggcgtggatgcccgccgcgtactcgccgctgcggtgcggggcgatgctctgggcgaactgcgcgccgcctcgggcacggtccatgacgagctcgtgggtctcgaggccgcgcagcagctcgccgagcccctggtggttgaggacgacttccacagtggacatcagccggtcaccctttccaggttgatcacgcacacgcccgagcccggcacgaacgggctcaggtagcgctggggtccgccggtcaccgcccagtgggtgccatccggcagcacgatccggtcggtggccagtacgtcggcctccggtccggtgtagacggtcatgcgggcggtgacctgatcggcgctgtcggtgtcctcggtcgaaccgcccggggccagcacgcagccggggatctcgtgctcggtgggcggcagcggatcgccgtagcgattggtgcccgccggtctggagaccgtgagtacctgccccgccaggccctcggggatcacgggacgtccaggatcgcgctgccggtgccgacgctgaaggccgcgccgagcccggctgccttccgtagccggatgcgctgttccctgctgagatccatgccctgggggcccccgctgtcggtataggtcgccgcgtacgggccgatcgtggtcgaggccgcaccgcccggcgtgggcaccgccgctttggccacctccagggccaccgcggtgacgtcgggcggcacagggtcgggcagctcgccgatctcgccgaggatcgccgcgagcgtccaggtgtggatcgctgcgccggtctcggccgagaccgggcggcccaccagcgcggccagggtctcggctgtgaacagctcggccatcacgtgcgccgcgttctccgggccttggccggggcgtccgtgccgggatcctcatccccgccggcctgacgtactccgagctcggccgggagggtgcgcagccgggccgccgtcgcggcgtcgacctcggccacgccatcggtgaaccgcacacccaaatcgcgcacgtacaggccgggatgccgctggcaggtgaacttcac

SEQ ID NO: 138 - DNA, 1070 bp, Saccharopolyspora spinosa
ctaaaccggtctgggcgacacctcgcaccaagcccagtcacgaatgaaggcctggcaggctaccgcgaagcctgccaggccgcttcggttgtcggtcagaagtcgaaggccaccgaggtgggtgttcttgatgcagtcgttgtcgaaggtccgcgagtagctgaccggcctgccctcccgcgtaccggtggcggtcacctccaccgggcgtcgcctccaccgggcgcgagatcatcgggcagggggcgtccggggattgcatgggaatgctgtcggaattcccccgaacccccggcagcagcacgcaggcgccagccgcgtccgggtacgagccaccagcagcgtcgcaggtgagcgtcacgctcgaccgcgcactctgcccccttcgtggtggtcaccccggagtaaaaccgcagcttccgggtgatttgcactccccccaccccggctatccaccccgccggatgtactcgtccacactggacttctctacggaaaaccccgcccgtgatggcaagttcggcggttgacccccggggcgatcgacctgctgaagatggacatcgagggcgccgaggccgacgcgctcgccggaatcggcgatcacgactgggccaggacccggcaggtcgtactcgaagtacatgacttcgccggtgccccggcggccgtgcgcaccacgctggcggaacgcggcttcaccatcaccgcggaccggccggagggcatgctaggcgggctgggtaccaagaccgtttacgcacggcggtgaacgcggcaagatgcggatcatcctgaccagcctgccctactactcccacctggtgccggttgtggtcccggtggcccacgccctgcggcgcgccgggcacaccgtcgccgtcgcgaccgcaccgtacatggcctcggaactctcgcggcactgcgtcgaacacctgccgctgccgaacgtccaaaccctcgaacagctgctcgccgccccgcgttcgtcaccagccccggcatgcccggagcggaaggggaagccgccgaggacaccgcgcgcacccgcgagaatcccggtccgctga

SEQ ID NO: 139 - DNA, 999 bp, Saccharopolyspora spinosa
atggaacatctgccgttcatcgcaacgctgcagccgagctcagctcggctgcggcgggcaaccaagtccgggcactgctgctccagctggaaagcccagggcaccgcagggttggagtgcattcacggatttccactgctgcagcgcgacgtgcgcgccgctgagcggcgagccaaacggatcccggtgccagaggcggccgagcgggaaccgtccgagtgcgtgtgactatgagccagtcctttcgtcctccggtgtcgtctgaatctccgcgccgctattcagtgccaccggtaaccccgcgcggcatatggatcgcgcggcacggcggtcaccgagtggtccacgtgcacctcgtgtgcaccgaggggtcctccacctcaccactgggcgttcaccggacgttgtgcgatctgcggctgggcgacgattgggttcacttccgccgtgtggctccattgccgagctggttgacggtgtgcccgctgtgcttggccagtcccaggatttcgctggacctcgccttgcgcatcgagcggcatctcatcagccgcgaaccgctgaccgccgcacggctgatggatcgccaagacgccaccggattgattccccgagttccgttcaccgacagtctggtattcgaggtcgatcgacatgctcgcgatggaacacccgtcattcgtgctggaacaagcgcgagcgcagcagcagcaccgtacgctgctgcctccggtgtacctcggcgacttccgcacggcgctatacagagcgatgccgaacgtccggtaccacagttgtcccgacgacggcgggatcgcgcacctattcatcatcgacgccggagctgccgactcgtgggtggtctggtggcagcgcgaagacggctcccacaccattgcggaagccggcccgtacgcgctccgcgaccgcatcaaccagatcgctggcgattggttgatccacatcgccaccaccgtggagatcgcgcacatcgcgtga

SEQ ID NO: 140 - DNA, 1103 bp, Saccharopolyspora spinosa
ttagcgctccatgaaaccggtcaagccgcccttgggctcggaccactgctccgcgacgtggtcgacgtggcccggggtttcgccggaacgcagcgcagtcaacagcttctcgcacgcttcccgggggccttcggcgaccacctcgacgcggttcgcgggccggttcgtggcgctgcccaccaggccgagttccagcgcccgggaacgcgtccaccagcggaagccgacgccctgcacccgcccgtgcacccatgcggtcagtcgtgcctgttgcgaatccgtcacgccgggcatgatgccgcacggccgctgcgcgcgcggccctggcttgcgccgatcatcaccactgcccgggcaccggtatgatccgccacgattcgtggtagggccgggtgttctggggcatccgcgcagaccgcccgacgagccgacgagtggggcaccgtggacccgatgttgccggaagacccggacgcccggccgaagcgcctggtgatgctggccgcatcgagcatcgccgtggccaccgtgttcgcgacgaccgccgcgatggtcgccggcgcgcagcagcacgacgcggcgacgccggacttcacgctgaagccgaccgccacgtcccggccggcctcgacgttctccacgccgtcttcgttgctgcccccgccgccgatgagcagcgaaacctcggatatctcgacgacctccagcaggtcgacttcgtccgcttcgaagagcagcgaaacgctcacggagatcccgcctccgatgccgccagagccgcccccgccccacactccgccgcggccgacgaagaccaccacgacacccacgaccacgacgaccaccaccacgacgaccacgaccaccacgaccacggagccgaccgactcagagacctccggcggtgacgactagtgacgttggccgtgcacgcctgccagtcgctgtgctcgtggcaccggactcccaagcagctcaacgggtttccgctgctggcctgtcgcggctgcgggtcgcagtggatccgcagcgagccgtggacgcccatcgaccacaccggccggatccccgacgacgttcgggcggagctcgccgagcgctga

SEQ ID NO: 141 - DNA, 1191 bp, Saccharopolyspora spinosa
ttaggccgtcgcgggttcggcttcgtccgtgcgggcccagcggttcggttcacccgtgatggcgaggcaggtgaccgtgatcagcgcgccgatcagcaggtaggccgcgatcggccaggacgccccgccggaggtcgtgagcagcgcggtagccaccagcggcgacaacccgccgcccagcaccgaaccgctctggtagccgagggaaacacccgtgtagcggaccttcgtggcgaacagtccggcgcagaacgccgccatcggcccgaaaaccgccgccgaaccggcatatcccagcgtcatggccagcacgatcagcgccgggctcgcggtgtccagcagccagaacatcggaaatgcgtacaacgcccggaaaatcccgccgccgagcatcacctgggtgcggcagatgaggtccgacaggtgcgagaacagcagccctcagcagcagccaattcgcccgcaaagctcgccctgactggctccgcctggtggccgacgtcgaggcgggccgcgtaaacgttttgatcatctgggagccgtcccgagcgtcccggctgttatctgcgtggtcgacgctgctggaaacctgccaacggctcggcgtactgatccacgtcaccagccaccagcagacctacgatctcgacaatccgcgccactggcgcacactcgccgaagacggcgttgacgcggtgtacgaagccgagaagacgtagcatcgcgtccgacggggcacaaagtcctccgcggcggccggccgaccgcacgggaaagtcctttacggctaccgccgggtgtacgacccagagacgcgcgcattgggcggaaacgacttcgacgccgacaagctcgtgtctcgcgtcgccgagacggtgcgccgcaccatcgctgacgccacgatcaacgtcgatgtgaacgtgcagcggggggatggcgccaatgtctgaccggctccgcgcctggctgcgcaccaccatccctgcggcatggtccgcgctcgtcgcgtggctgatcgcggccggcgtcccggactggctcaccggcccgctcggcgcggccggtgacgtcctggtcgtactcggcgccctctacgcgctgttgcgctggaccgagccgcacatgccgccgtggctgacccggatactgctcggctccaacacaccacccacctacccgccgaccgagtag

SEQ ID NO: 142 - DNA, 951 bp, Saccharopolyspora spinosa
atgaccggcttgacggtcgatccgctcgacccggcggtggtcccgctccgcgagggccggaccgtcctgggcgcggggttcctggtcgcaccgggtgtggtcgccacctgcgcccacgtggtcggcagagcaacgccggtcgccgatttcccgttgctgcgcggccacgaccacgccgtcgaagtgctgtcgcaggacgacgacctggacgtcgcgatcctgcggctggcggacacaccaccgggagcgctgccggttccggcacgcagaccgcgccgacgttggagctcgacgtgcggcccgcctgggagtcgccgccaggtggtccgaagatcgcgctccgcccgggagatccggaccagtcgctggcgccagaccagcgacggcctctcggtgtgggacctgcggaccagaacgcaggcgcacgcgttccggctcggggtcaccgacctggtcgtgtccgccgacggctccgccgccgcgatgacggaccaggcgaacaactcgatcgggctggtcgacctgatcaagatggattagatcgcgccgctgatcgccccgggcctgagactcatcgggatgtccgtgccgtacttgttcgccaaggacgtcaccgcggtgcaggtccgtttcggactacaagcacgcaaatcgctcaagacgttcccgctggacgggttgtccgtggccgatcgaccggtcgtcacccggacgggtccgtggccacgacgatcaccggcgattcagttgctttgatcgacctggagaacatggccccgttgccgccgctgatcggcaaggtcgacgagatcgaggcctctccggcggctacctgatctccgaggagaccctggacttccaggtctggaacttgcaggaacgccgcctggtcaccgcgatcaccctcgacgacacggattcgcggcgcgccgtcgagaacggcgatctcat

SEQ ID NO: 143 - protein, 236 aa, Artificial Sequence
  codon optimized reporter gene DasherGFP
MTALTEGAKLFEKEIPYITELEGDVEGMKFIIKGEGTGDATTGTIKAKYICTTGDLPVPWATLVSTLSYGVQCFAKYPSHIKDFFKSAMPEGYTQERTISFEGDGVYKTRAMVTYERGSIYNRVTLTGENFKKDGHILRKNVAFQCPPSILYILPDTVNNGIRVEFNQAYDIEGVTEKLVTKCSQMNRPLAGSAAVHIPRYHHITYHTKLSKDRDERRDHMCLVEVVKAVDLDTYQ

SEQ ID NO: 144 - protein, 263 aa, Artificial Sequence
  codon optimized reporter gene PaprikaRFP
MVSKGEELIKENMRMKLYMEGTVNNHHFKCTSEGEGKPYEGTQTMRIKVVEGGPLPFAFDILATSFMYGSRTFIKYPKGIPDFFKQSFPEGFTWERVTRYEDGGVVTVMQDTSLEDGCLVYHVQVRGVNFPSNGPVMQKKTKGWEPNTEMLYPADGGLEGRSDMALKLVGGGHLSCSFVTTYRSKKPAKNLKMPGIHAVDHRLERLEESDNEMFVVQREHAVARYCDLPSKLGHKLNSGLRSRAQASNSAVDGTAGPGSTGSR

SEQ ID NO: 145 - protein, 603 aa, Artificial Sequence
  codon optimized reporter gene gusA
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQGILRVGGNKKGIFTRDRKPKSAAFLLQKRWTGMNFGEKPQQGGKQ

SEQ ID NO: 146 - DNA, 1422 bp, Artificial Sequence
  codon optimized sacB gene
atgaacatcaagaagttcgccaagcgggcgaccgtcctgaccttcaccaccgccctgctcgcgggcggggccacccaggccttcgccaaggagaacacccagaagccctacaaggagacgtacggggtgtcgcacatcacccgccacgacatgctccagatccccaagcagcagcagagcgagaagtaccaggtcccgcagttcgaccagtccaccatcaagaacatcgaatcggccaagggcctcgacgtgtgggactcctggcccctgcagaacgccgacggcaccgtggccgagtacaacgggtaccacgtggtgttcgccctggcgggctcccccaaggacgccgacgacacctcgatctacatgttctaccagaaggtcggcgacaacagcatcgactcctggaagaacgcgggccgcgtcttcaaggacagcgacaagttcgacgcgaacgacgagatcctgaaggagcagacccaggagtggtccggctccgccaccttcacgtccgacggcaagatccggctcttctacacggacttctccggcacgcactacgggaagcagagcctcaccacggcgcaggtcaacgtgtcgaagtccgacgacaccctcaagatcaacggcgtggaggaccacaagacgatcttcgacggcgacggcaagacctaccagaacgtgcagcagttcatcgacgagggcaactacacgtcgggcgacaaccacacgctgcgcgacccccactacgtggaggacaaggggcacaagtacctggtcttcgaggccaacaccggcaccgacaacggctaccagggcgaggaatccctgttcaacaaggcgtactacggcggcagcacgaacttcttccgcaaggagagccagaagctccagcagtcggccaagaagcgggacgccgagctcgccaacggcgcgctgggcatggtggagctgaacgacgactacacgctgaagaaggtcatgaagccgctcatcacctccaacaccgtgacggacgagatcgagcgggcgaacgtcttcaagatgaacggcaagtggtacctgttcaccgactcccgcggctccaagatgaccatcgacggcatcaactcgaacgacatctacatgctgggttacgtctccaacagcctgaccgggccgtacaagccgctcaacaagaccggcctggtgctccagatgggcctggacccgaacgacgtcaccttcacctactcccacttcgcggtgccccaggcgaagggcaacaacgtggtcatcacctcgtacatgacgaaccggggcttcttcgaggacaagaaggccaccttcgccccctccttcctgatgaacatcaagggcaagaagacctccgtggtgaagaacagcatcctggagcagggccagctcaccgtcaacaactga

SEQ ID NO: 147 - DNA, 1068 bp, Artificial Sequence
  codon optimized S. erythraea pheS gene
atgtccggtgcgaacgacccctacgaccccaagcaggtggccgcgctgtccgccgaaaccctggaacgggcggtggccgacgcgcgggaagccttcgacaaggccggtgacctcgacgaactggccgccgccaagccggcccacctgggtgaacggagcccgctgctgacggcccggcgggagatcggtgccctgcccccgaaggcccgctccgacgcgggtaagcgcgtgaacgaggcgcgggaggcgatccagggcgccttcgacgagcggcgggcggccctccaggcggaacgcgacgaacgggtgctgcgcgaagaagccgtcgacgtcaccctcccctgggaccgcgtgcccgtgggtgcgcgccacccgatcacccagctgatcgagcacgtggccgacacgttcgtggccatgggttgggaagtcgccgaaggccccgagctcgagaccgaatggttcaacttcgacgccctgaacttcggcaaggaccacccggcgcgcaccatgcaggacaccttctacgtcggtccgaaggaatccggtctcgtcctccggacgcacaccagcccggcccaggtccgcgccctgctggaccgggaactgccggtgtacgtggtgtgccccggccgcaccttccggaccgacgagctggactccacgcacacgccggtcttccaccaggtggaagggctcgccgtggacaagggtctcacgatggcccacctcaagggcacgctcgacgcgttcgcgcgcgtcatgttcggtcccgaatccaagacgcgcctgcgcccgagcttcttcccgttcgccgaaccctcgggtgaagtcgacgtctggttcccgcagaagaagggcggtcccggctgggtcgaatggggcggctgcggtatggtcaacccgaacgtcctgcgcgcctgcggtgtcgaccccgaaacccacaccggtttcggcttcgggatgggtctcgaacggacgctccagttccgcaacggtatcccggacatgcgggacatggtggaaggtgacgtgcagttcacgcagcccttcggtatcgactcctga

SEQ ID NO: 148 - DNA, 1062 bp, Artificial Sequence
  codon optimized S. spinosa pheS gene
gtgtccggcgccaacgacccgtacgacccgaaggaagtggcggcgctctcgccggagacgctggatcgcgcggtggtcgaggcgagcaaggcgttcgccacggcgacggacctggacgcgctcgccgtggtgaagccggcgcatctcggcgatcgtagcccgctgctcaccgcgcgtcgcgaaatcggtgcgctgccgcccaaggcgcgcagcgaagcgggcaagcgcgtgaatgaagcgcgcgaggccatccagtcggcgttcgacgagcgccgcgccgccttgcaggctgagcgcgatgaacgggtcctccgcgaggagaccgttgacgtgaccctgccgtgggaccgggtctccgcgggggcccgccacccgatcacccagctggctgaggatattgaagacacgttcgtggcgatgggttgggaggtcgcggaggggccggagttggaagccgaatggttcaatttcgacgccctgaacttcggtaaggatcatccggcgcgcacgatgcaggacaccttctatgtcgcccccgaaaactcggggctggtcttgcggacccacacgtccccgtcgcaggtccgggccctcctggatcgcgagctgccggtttacgtggtttgtcccggccgtaccttccggacggacgaattggatgcgacccacacgccggtctttagccaagttgaagggctggcggttgacaagggtctgagcatggcccacttgaaggggacgctggatgcgtttgcgcggtcgatgttcggtccggaatcgaagacccggctgcggccgtcgtacttcccgttttcggagccgagcgcggaaatggacgtgtggttcccggagaagaaggggggcgcgggctgggtggagtggggagggtgtggtatggtcaaccccaacgtgctccgcgcgtgcggcgtggacccggaggtctacaccggtttcggtttcggtatgggcctggagcggaccctgatgttccgcaacggcatcccggacatgcgggatatggtcgagggggatgtgcgtttcacgcagccgtttgggatctga

SEQ ID NO: 149 - DNA, 309 bp, Saccharopolyspora erythraea
agcttggtaccagcccgacccgagcacgcgccggcacgcctggtcgatgtcggaccggagttcgaggtacgcggcttgcaggtccaggaaggggacgtccatgcgagtgtccgttcgagtggcggcttgcgcccgatgctagtcgcggttgatcggcgatcgcaggtgcacgcggtcgatcttgacggctggcgagaggtgcggggaggatctgaccgacgcggtccacacgtggcaccgcgatgctgttgtgggcacaatcgtgccggttggtaggatccccacccaacgcaccccaggaggtcccat

SEQ ID NO: 150 - DNA, 77 bp, Artificial Sequence
  terminator sequence for GFP, RFP
atcgatagccgccccgcagggcgctccgcaggccgcttccggaccactccggaagcggccgtgcggtcggaggtacc

SEQ ID NO: 151 - protein, 261 aa, Artificial Sequence
  selection marker gene aac(3)IV conferring resistance to Apramycin
MQYEWRKAELIGQLLNLGVTPGGVLLVHSSFRSVRPLEDGPLGLIEALRAALGPGGTLVMPSWSGLDDEPFDPATSPVTPDLGVVSDTFWRLPNVKRSAHPFAFAAAGPQAEQIISDPLPLPPHSPASPVARVHELDGQVLLLGVGHDANTTLHLAELMAKVPYGVPRHCTILQDGKLVRVDYLENDHCCERFALADRWLKEKSLQKEGPVGHAFARLIRSRDIVATALGQLGRDPLIFLHPPEGGMRRMRCRSPVDWLSS

SEQ ID NO: 152 - protein, 177 aa, Artificial Sequence
  selection marker gene aacC1 conferring resistance to Gentamycin
MLRSSNDVTQQGSRPKTKLGGSSMGIIRTCRLGPDQVKSMRAALDLFGREFGDVATYSQHQPDSDYLGNLLRSKTFIALAAFDQEAVVGALAAYVLPRFEQPRSEIYIYDLAVSGEHRRQGIATALINLLKHEANALGAYVIYVQADYGDDPAVALYTKLGIREEVMHFDIDPSTAT

SEQ ID NO: 153 - protein, 286 aa, Artificial Sequence
  selection marker gene aacC8 conferring resistance to Neomycin B
MDEKELIERAGGPVTRGRLVRDLEALGVGAGDTVMVHTRMSAIGYVVGGPQTVIDAVRDAVGADGTLMAYCGWNDAPPYDLAEWPPAWREAARAEWPAYDPLLSEADRGNGRVPEALRHQPGAVRSRHPDASFVAVGPAAHPLMDDHPWDDPHGPDSPLARLAGAGGRVLLLGAPLDTLTLLHHAEARAEAPGKRFVAYEQPVTVGGRRVWRRFRDVDTSRGVPYGRVVPEGVVPFTVIAQDMLAAGIGRTGRVAAAPVHLFEAADVVRFGVEWIESRMGGAAGGA

SEQ ID NO: 154 - protein, 262 aa, Artificial Sequence
  selection marker gene aadA conferring resistance to Spectinomycin, Streptomycin
MREAVIAEVSTQLSEVVGVIERHLEPTLLAVHLYGSAVDGGLKPHSDIDLLVTVTVRLDETTRRALINDLLETSASPGESEILRAVEVTIVVHDDIIPWRYPAKRELQFGEWQRNDILAGIFEPATIDIDLAILLTKAREHSVALVGPAAEELFDPVPEQDLFEALNETLTLWNSPPDWAGDERNVVLTLSRIWYSAVTGKIAPKDVAADWAMERLPAQYQPVILEARQAYLGQEEDRLASRADQLEEFVHYVKGEITKVVG

SEQ ID NO: 155 - protein, 126 aa, Artificial Sequence
  selection marker gene ble conferring resistance to Bleomycin
MTDQATPNLPSRDFDSTAAFYERLGFGIVFRDAGWMILQRGDLMLEFFAHPGLDPLASWFSCCLRLDDLAEFYRQCKSVGIQETSSGYPRIHAPELQEWGGTMAALVDPDGTLLRLIQNELLAGIS

SEQ ID NO: 156 - protein, 219 aa, Artificial Sequence
  selection marker gene cat conferring resistance to Chloramphenicol
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 157 - protein, 381 aa, Artificial Sequence
  selection marker gene ermE conferring resistance to Erythromycin
MSSSDEQPRPRRRNQDRQHPNQNRPVLGRTERDRNRRQFGQNFLRDRKTIARIAETAELRPDLPVLEAGPGEGLLTRELADRARQVTSYEIDPRLAKSLREKLSGHPNIEVVNADFLTAEPPPEPFAFVGAIPYGITSAIVDWCLEAPTIETATMVTQLEFARKRTGDYGRWSRLTVMTWPLFEWEFVEKVDRRLFKPVPKVDSAIMRLRRRAEPLLEGAALERYESMVELCFTGVGGNIQASLLRKYPRRRVEAALDHAGVGGGAVVAYVRPEQWLRLFERLDQKNEPRGGQPQRGRRTGGRDHGDRRTGGQDRGDRRTGGRDHRDRQASGHGDRRSSGRNRDDGRTGEREQGDQGGRRGPSGGGRTGGRPGRRGGPGQR

SEQ ID NO: 158 - protein, 332 aa, Artificial Sequence
  selection marker gene hyg onferring resistance to Hygromycin
MTQESLLLLDRIDSDDSYASLRNDQEFWEPLARRALEELGLPVPPVLRVPGESTNPVLVGEPDPVIKLFGEHWCGPESLASESEAYAVLADAPVPVPRLLGRGELRPGTGAWPWPYLVMSRMTGTTWRSAMDGTTDRNALLALARELGRVLGRLHRVPLTGNTVLTPHSEVFPELLRERRAATVEDHRGWGYLSPRLLDRLEDWLPDVDTLLAGREPRFVHGDLHGTNIFVDLAATEVTGIVDFTDVYAGDSRYSLVQLHLNAFRGDREILAALLDGAQWKRTEDFARELLAFTFLHDFEVFEETPLDLSGFTDPEELAQFLWGPPDTAPGA

SEQ ID NO: 159 - protein, 264 aa, Artificial Sequence [sequence omitted]

SEQ ID NO: 160 - DNA, 1644 bp, Artificial Sequence
  selection marker gene amdSYM
atgccccagtcctgggaggagctggcggccgacaagcgcgcgcgcctcgcgaagacgatcccggacgagtggaaggtccagacgctgcccgcggaggactccgtgatcgacttccccaagaagtcggggatcctctccgaggcggagctgaagatcaccgaagcctccgcggccgacctggtcagcaagctggcggccggcgagctgaccagcgtcgaagtcaccctggccttctgcaagcgggcggccatcgcgcagcagctcacgaactgcgcccacgagttcttccccgacgccgccctcgcccaggcgcgcgagctggacgagtactacgccaagcacaagcgcccggtggggcccctccacgggctgccgatctcgctgaaggaccagctccgggtcaagggctacgagacctcgatggggtacatctcgtggctgaacaagtacgacgagggcgactccgtcctgaccaccatgctgcggaaggccggcgccgtcttctacgtcaagacctcggtcccgcagaccctcatggtgtgcgagacggtgaacaacatcatcggccggaccgtgaacccccggaacaagaactggtcctgcggcggctcctccggcggggagggggccatcgtcggcatccgcggcggcgtcatcggcgtgggcaccgacatcggcggctccatccgggtgcccgccgccttcaacttcctctacggcctgcgcccgtcccacgggcgcctcccgtacgcgaagatggccaactccatggagggccaggagaccgtgcactcggtggtgggccccatcacccactcggtcgaagacctgcgcctgttcacgaagagcgtcctgggccaggaaccgtggaagtacgacagcaaggtgatcccgatgccgtggcgccagtccgagtcggacatcatcgcctccaagatcaagaacgggggcctgaacatcgggtactacaacttcgacggcaacgtgctcccgcaccccccgatcctgcgcggggtcgagaccacggtggccgccctggccaaggccggccacaccgtcacgccctggaccccgtacaagcacgacttcggccacgacctcatctcccacatctacgcggcggacggcagcgccgacgtgatgcgcgacatctcggcctccggggaaccggcgatccccaacatcaaggacctgctgaaccccaacatcaaggccgtcaacatgaacgagctgtgggacacccacctgcagaagtggaactaccagatggaatacctcgagaagtggcgcgaggccgaggagaaggcgggcaaggagctggacgcgatcatcgccccgatcacccccaccgcggcggtgcggcacgaccagttccggtactacggctacgcctcggtcatcaacctcctggacttcacctccgtcgtcgtcccggtgacgttcgcggacaagaacatcgacaagaagaacgaatcgttcaaggcggtctcggagctggacgccctcgtgcaggaggagtacgacccggaagcctaccacggcgccccggtcgccgtccaggtgatcgggcgccgcctgtcggaggagcgcaccctcgcgatcgccgaggaggtgggcaagctgctgggcaacgtcgtgacgccc

SEQ ID NO: 161 - DNA, 1215 bp, Artificial Sequence
  Counter selection marker tetA gene
atgaaccgcaccgtgatgatggcgctcgtcatcatcttcctcgacgccatgggcatcggcatcatcatgccggtcctgccggccctgctgcgggagttcgtgggcaaggcgaacgtggcggagaactacggcgtcctcctcgcgctgtacgccatgatgcaggtgatcttcgcgccgctgctcggccggtggtcggaccgcatcggccgccggccggtcctgctgctctcgctcctcggggcgaccctggactacgccctcatggcgacggcgtccgtcgtgtgggtcctctacctgggccggctgatcgccggcatcacgggcgccaccggcgccgtcgcggcgtcgacgatcgccgacgtcaccccggaggagtcgcgcacccactggttcggcatgatgggcgcctgcttcggcggcgggatgatcgccggccccgtgatcggcggcttcgccggccagctctcggtgcaggcccccttcatgttcgccgccgccatcaacggcctggccttcctggtgtcgctgttcatcctgcacgagacccacaacgccaaccaggtgtccgacgaactgaagaacgaaaccatcaacgagacgacctcgtcgatccgggagatgatctcccccctgtccggcctgctcgtggtcttcttcatcatccagctgatcggccagatccccgcgaccctctgggtgctgttcggcgaggaacgcttcgcctgggacggcgtgatggtgggggtgtccctcgcggtgttcgggctcacccacgcgctgttccagggcctggcggcgggcttcatcgccaagcacctgggcgagcgcaaggccatcgccgtcgggatcctggccgacggctgcggcctgttcctgctggcggtgatcacccagtcctggatggtctggccggtcctgctgctcctggcctgcggcgggatcaccctgccggcgctccagggcatcatctccgtccgcgtcggccaggtcgcgcaggggcagctgcagggcgtgctgacgtcgctcacccacctcacggccgtgatcgggccgctcgtgttcgccttcctgtactccgcgacccgcgagacctggaacggctgggtgtggatcatcggctgcggcctgtacgtcgtggccctcatcatcctgcgcttcttccaccccggccgggtgatccaccccatcaacaagtccgacgtccagcagcggatc

SEQ ID NO: 162 - DNA, 1251 bp, Artificial Sequence
  Counter selection marker lacY gene
atgtactacctgaagaacaccaacttctggatgttcggcctgttcttcttcttctacttcttcatcatgggcgcctacttcccgttcttccccatctggctgcacgacatcaaccacatctcgaagtcggacaccgggatcatcttcgcggccatctcgctcttctcgctcctgttccagccgctcttcgggctgctctcggacaagctgggcctccgcaagtacctgctctggatcatcacgggcatgctggtcatgttcgcgccgttcttcatcttcatcttcggccccctcctgcagtacaacatcctggtggggtcgatcgtcggcgggatctacctgggcttctgcttcaacgccggggcgccggccgtcgaggccttcatcgagaaggtctcgcgccggtcgaacttcgagttcgggcgcgcccggatgttcggctgcgtgggctgggccctctgcgcctccatcgtgggcatcatgttcacgatcaacaaccagttcgtcttctggctggggtccggctgcgccctcatcctcgcggtgctgctgttcttcgccaagaccgacgccccgagcagcgcgacggtcgcgaacgccgtgggggcgaaccactccgccttctcgctcaagctcgcgctggagctgttccggcagcccaagctgtggttcctgtcgctgtacgtcatcggcgtcagctgcacgtacgacgtgttcgaccagcagttcgccaacttcttcacctcgttcttcgccaccggcgagcagggcacccgggtcttcggctacgtgaccacgatgggggagctgctcaacgcctcgatcatgttcttcgcccccctgatcatcaaccgcatcggcggcaagaacgccctcctcctggccggcaccatcatgtccgtccgcatcatcggctccagcttcgcgacctccgccctggaggtcgtgatcctgaagaccctgcacatgttcgaggtcccgttcctcctggtcggctgcttcaagtacatcacctcccagttcgaggtccgcttctcggccacgatctacctggtctgcttctgcttcttcaagcagctggcgatgatcttcatgagcgtcctcgcgggcaacatgtacgaaagcatcggcttccagggcgcctacctggtgctgggcctggtggccctcggcttcaccctcatcagcgtcttcaccctctccggcccgggcccgctgtccctgctccgccgccaggtcaacgaggtggcg

SEQ ID NO: 163 - DNA, 1419 bp, Artificial Sequence
  Counter selection marker sacB gene
atgaacatcaagaagttcgccaagcgggcgaccgtcctgaccttcaccaccgccctgctcgcgggcggggccacccaggccttcgccaaggagaacacccagaagccctacaaggagacgtacggggtgtcgcacatcacccgccacgacatgctccagatccccaagcagcagcagagcgagaagtaccaggtcccgcagttcgaccagtccaccatcaagaacatcgaatcggccaagggcctcgacgtgtgggactcctggcccctgcagaacgccgacggcaccgtggccgagtacaacgggtaccacgtggtgttcgccctggcgggctcccccaaggacgccgacgacacctcgatctacatgttctaccagaaggtcggcgacaacagcatcgactcctggaagaacgcgggccgcgtcttcaaggacagcgacaagttcgacgcgaacgacgagatcctgaaggagcagacccaggagtggtccggctccgccaccttcacgtccgacggcaagatccggctcttctacacggacttctccggcacgcactacgggaagcagagcctcaccacggcgcaggtcaacgtgtcgaagtccgacgacaccctcaagatcaacggcgtggaggaccacaagacgatcttcgacggcgacggcaagacctaccagaacgtgcagcagttcatcgacgagggcaactacacgtcgggcgacaaccacacgctgcgcgacccccactacgtggaggacaaggggcacaagtacctggtcttcgaggccaacaccggcaccgacaacggctaccagggcgaggaatccctgttcaacaaggcgtactacggcggcagcacgaacttcttccgcaaggagagccagaagctccagcagtcggccaagaagcgggacgccgagctcgccaacggcgcgctgggcatggtggagctgaacgacgactacacgctgaagaaggtcatgaagccgctcatcacctccaacaccgtgacggacgagatcgagcgggcgaacgtcttcaagatgaacggcaagtggtacctgttcaccgactcccgcggctccaagatgaccatcgacggcatcaactcgaacgacatctacatgctgggctacgtctccaacagcctgaccgggccgtacaagccgctcaacaagaccggcctggtgctccagatgggcctggacccgaacgacgtcaccttcacctactcccacttcgcggtgccccaggcgaagggcaacaacgtggtcatcacctcgtacatgacgaaccggggcttcttcgaggacaagaaggccaccttcgccccctccttcctgatgaacatcaagggcaagaagacctccgtggtgaagaacagcatcctggagcagggccagctcaccgtcaacaac

SEQ ID NO: 164 - DNA, 1068 bp, Artificial Sequence; identical to SEQ ID NO: 147
  Counter selection marker pheS gene derived from S. erythraea

SEQ ID NO: 165 - DNA, 1038 bp, Artificial Sequence
  Counter selection marker pheS gene derived from Corynebacterium
atgagcgagatccagctgaccgaggcctcgctcaacgaggccgccgacgccgccatcaaggccttcgacggcgcgcagaacctcgacgaactcgcggcgctgcgccgggaccacctgggcgacgccgcccccatcccgcaggcccgccgctccctcgggaccatcccgaaggaccagcgcaaggacgcgggtcgcttcgtgaacatggccctcggtcgcgcggaaaagcacttcgcccaggtcaaggtggtgctcgaagaaaagcgcaacgcggaggtcctcgagctcgaacgggtggacgtgaccgtcccgaccacccgggaacaggtgggtgcgctccacccgatcaccatcctgaacgaacagatcgcggacatcttcgtcggtatgggctgggaaatcgccgaaggtccggaggtggaggcggagtacttcaacttcgacgcgctcaacttcctccccgaccaccccgcgcgcacgctccaggacaccttccacatcgcgcccgagggttcgcgccaggtgctgcggacgcacacctccccggtgcaggtccgcaccatgctgaaccgcgaagtgcccatctacatcgcgtgcccgggtcgggtgttccgcacggacgaactcgacgcgacccacaccccggtcttccaccagatcgaggggctggcggtcgacaagggtctgacgatggcccacctgcgcgggacgctggaccacctggccaaggagctgttcgggccggaaaccaagacgcgcatgcgctccaactacttcccgttctcggagccctccgcggaggtcgacgtctggttcccgaacaagaagggtggggccggctggatcgaatggggcgggtgcggcatggtgaaccccaacgtcctccgcgccgtgggtgtcgacccggaagagtacaccgggttcggcttcggcatgggcatcgaacggaccctgcagttccgcaacggtctgagcgacatgcgggacatggtcgagggtgacatccggttcacgctcccgttcggcatccaggcctga

SEQ ID NO: 166 - DNA, 668 bp, Artificial Sequence
  GST Solubility Tag
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttcc

SEQ ID NO: 167 - DNA, 150 bp, Saccharopolyspora endophytica
gcgagaggcccggaagcgagatcgcttccgggcctctgacctgcggaggatacgggattcgaacccgtgagggctattaacccaacacgatttccaattccgatggcgcgagtgccagggggtagctgaacgtgccttttgcctggtcag

SEQ ID NO: 168 - DNA, 45 bp, Saccharopolyspora erythraea
tcggagccgctgaggggactcgaacccctgaccgtccgcttacaa

SEQ ID NO: 169 - DNA, 52 bp, Saccharopolyspora spinosa
ggcagctcttggtggtggccaggggcggggtcgaaccgccgaccttccgctt

SEQ ID NO: 170 - DNA, 48 bp, Saccharopolyspora spinosa
tcggagccgctgaggggactcgaacccctgaccgtccgcttacaaggc

SEQ ID NO: 171 - DNA, 76 bp, Saccharopolyspora spinosa
ggagccgcctaagggaatcgaacccttgacctacgcattacgagtgcgtcgctctagccgactgagctaaggcggc

SEQ ID NO: 172 - DNA, 41 bp, Artificial Sequence
  synthetic promoter Pmut-1
tgtgcggtggctaacacgtcctagtatggtatcatgagcaa

SEQ ID NO: 173 - DNA, 41 bp, Artificial Sequence
  synthetic promoter B2
tgtgcgctggctaacacgtcctagtatggtatagtgagcaa

SEQ ID NO: 174 - DNA, 41 bp, Artificial Sequence
  synthetic promoter D1
tgtgcggttcctaacacgtcctagtatggtactatgagcaa

SEQ ID NO: 175 - DNA, 41 bp, Artificial Sequence; identical to SEQ ID NO: 172
  synthetic promoter D2

(19) United States
(12) Patent Application Publication
(10) Pub. No.: US 2020/0115705 A1
(43) Pub. Date: Apr. 16, 2020

(54) A HIGH-THROUGHPUT (HTP) GENOMIC ENGINEERING PLATFORM FOR IMPROVING SACCHAROPOLYSPORA SPINOSA

(71) Applicant: Zymergen Inc., Emeryville, CA (US)

(72) Inventors: Benjamin MASON, San Francisco, CA (US); Alexi GORANOV, Oakland, CA (US); Peter KELLY, Oakland, CA (US); Youngnyun KIM, Oakland, CA (US); Sheetal MODI, Emeryville, CA (US); Nihal PASUMARTHI, Emeryville, CA (US); Benjamin MIJTS, Emeryville, CA (US); Peter ENYEART, Berkeley, CA (US)

(73) Assignee: Zymergen Inc., Emeryville, CA (US)

(21) Appl. No.: 16/620,203

(22) PCT Filed: Jun. 6, 2018
(86) PCT No.: PCT/US2018/036352

§ 371 Date: Dec. 6, 2019

Related U.S. Application Data
(60) Provisional application No. 62/515,934, filed on Jun. 6, 2017.

(51) Int. Cl. C12N 15/10; C12N 15/90; C12N 15/74; C12N 1/20

(52) U.S. Cl. CPC C12N 15/1079; C12N 15/902; C12N 1/20; C12N 15/1058; C12N 15/74

(57) ABSTRACT

The present disclosure provides a HTP microbial genomic engineering platform for Saccharopolyspora spp. that is computationally driven and integrates molecular biology, automation, and advanced machine learning protocols. This integrative platform utilizes a suite of HTP molecular tool sets to create HTP genetic design libraries, which are derived from, inter alia, scientific insight and iterative pattern recognition.

### CROSS-REFERENCE TO RELATED APPLICATION

The present application claims the benefit of priority from U.S. Provisional Patent Application Ser. No. 62/515,934 filed Jun. 6, 2017, which is herein incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ZYMR_013_01 WO_SeqList_ST25.txt. The text file is about 185 KB, was created on Jun. 6, 2018, and is being submitted electronically via EFS-Web.

### FIELD

The present disclosure is directed to high-throughput (HTP) microbial genomic engineering. The disclosed HTP genomic engineering platform is computationally driven and integrates molecular biology, automation, and advanced machine learning protocols. This integrative platform utilizes a suite of HTP molecular tool sets to create HTP genetic design libraries, which are derived from, inter alia, scientific insight and iterative pattern recognition. In particular, the taught platform is capable of performing HTP microbial genomic engineering in heretofore intractable microbial species.

### BACKGROUND

Humans have been harnessing the power of microbial cellular biosynthetic pathways for millennia to produce products of interest, the oldest examples of which include alcohol, vinegar, cheese, and yogurt. These products are still in large demand today and have also been accompanied by an ever increasing repertoire of products producible by microbes. The advent of genetic engineering technology has enabled scientists to design and program novel biosynthetic pathways into a variety of organisms to produce a broad range of industrial, medical, and consumer products. Indeed, microbial cellular cultures are now used to produce products ranging from small molecules, antibiotics, vaccines, insecticides, enzymes, fuels, and industrial chemicals.

Given the large number of products produced by modern industrial microbes, it comes as no surprise that engineers are under tremendous pressure to improve the speed and efficiency by which a given microorganism is able to produce a target product.

A variety of approaches have been used to improve the economy of biologically-based industrial processes by “improving” the microorganism involved. For example, many pharmaceutical and chemical industries rely on microbial strain improvement programs in which the parent strains of a microbial culture are continuously mutated through exposure to chemicals or UV radiation and are subsequently screened for performance increases, such as in productivity, yield and titer. This mutagenesis process is extensively repeated until a strain demonstrates a suitable increase in product performance. The subsequent “improved” strain is then utilized in commercial production.

As alluded to above, identification of improved industrial microbial strains through mutagenesis is time consuming and inefficient. The process, by its very nature, is haphazard and relies upon one stumbling upon a mutation that has a desirable outcome on product output.

Not only are traditional microbial strain improvement programs inefficient, but the process can also lead to industrial strains with a high degree of detrimental mutagenic load. The accumulation of mutations in industrial strains subjected to these types of programs can become significant and may lead to an eventual stagnation in the rate of performance improvement.

This is particularly an issue for microorganisms that many researchers consider “intractable,” i.e. those organisms for which traditional strain engineering tools are either not available or simply not functional. Once such group, the Saccharopolyspora spp., are notoriously difficult organisms to engineer. This is because compared to model system microbes, for which extensive studies have been carried out, and genomic engineering tools are readily available, many important tools for Saccharopolyspora spp. are yet to be created, tested, and/or improved.

Thus, Saccharopolyspora spp. present unique challenges for researchers attempting to improve the microbe for production purposes. These challenges have hampered the field of genomic engineering in Saccharopolyspora spp. and prevented researchers from harnessing the full potential of this microbial system.

Thus, there is a great need in the art for new methods of engineering industrial microbes, which do not suffer from the aforementioned drawbacks inherent with traditional strain improvement programs and greatly accelerate the process of discovering and consolidating beneficial mutations.

Further, there is an urgent need for a method by which to “rehabilitate” industrial strains that have been developed by the antiquated and deleterious processes currently employed in the field of microbial strain improvement.

In addition, the art desperately tools and processes, which are able to perform a HTP genomic engineering process in a traditionally intractable microbial species. Once such genera of microbial species, for which no HTP genomic engineering process is currently available, are the Saccharopolyspora spp.

### SUMMARY OF THE DISCLOSURE

The present disclosure provides a high-throughput (HTP) microbial genomic engineering platform that does not suffer from the myriad of problems associated with traditional microbial strain improvement programs.

Further, the HTP platform taught herein is able to rehabilitate industrial microbes that have accumulated non-beneficial mutations through decades of random mutagenesis-based strain improvement programs.

The HTP platform described herein provides novel microbial engineering tools and processes, which enable researchers to perform HTP genomic engineering in traditionally intractable microbial organisms. For example, the taught platform is the first of its kind that enables HTP genomic engineering in Saccharopolyspora spp. Until now, this group of organisms was not amenable to HTP genomic engineering. Consequently, the disclosed platform will revolutionize the field of genomic engineering in this organismal system.

The disclosed HTP genomic engineering platform is computationally driven and integrates molecular biology, automation, and advanced machine learning protocols. This integrative platform utilizes a suite of HTP molecular tool sets to create HTP genetic design libraries, which are derived from, inter alia, scientific insight and iterative pattern recognition.

The taught HTP genetic design libraries function as drivers of the genomic engineering process, by providing libraries of particular genomic alterations for testing in a microbe. The microbes engineered utilizing a particular library, or combination of libraries, are efficiently screened in a HTP manner for a resultant outcome, e.g. production of a product of interest. This process of utilizing the HTP genetic design libraries to define particular genomic alterations for testing in a microbe and then subsequently screening host microbial genomes harboring the alterations is implemented in an efficient and iterative manner. In some aspects, the iterative cycle or “rounds” of genomic engineering campaigns can be at least 1, 2, 3, 4, 5, 6, 7, 8, 9, 10, 20, 30, 40, 50, 60, 70, 80, 90, 100, or more iterations/cycles/rounds.

Thus, in some aspects, the present disclosure teaches methods of conducting at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 or more “rounds” of HTP genetic engineering (e.g., rounds of SNP swap, PRO swap, STOP swap, or combinations thereof).

In some embodiments, the present disclosure teaches a linear approach, in which each subsequent HTP genetic engineering round is based on genetic variation identified in the previous round of genetic engineering. In other embodiments the present disclosure teaches a non-linear approach, in which each subsequent HTP genetic engineering round is based on genetic variation identified in any previous round of genetic engineering, including previously conducted analysis, and separate HTP genetic engineering branches.

The data from these iterative cycles enables large scale data analytics and pattern recognition, which is utilized by the integrative platform to inform subsequent rounds of HTP genetic design library implementation. Consequently, the HTP genetic design libraries utilized in the taught platform are highly dynamic tools that benefit from large scale data pattern recognition algorithms and become more informative through each iterative round of microbial engineering.

In some embodiments, the genetic design libraries of the present disclosure comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 or more individual genetic changes (e.g., at least X number of promoter:gene combinations in the PRO swap library).

In some embodiments, the present disclosure provides illustrative examples and text describing application of HTP strain improvement methods to microbial strains. In some embodiments, the strain improvement methods of the present disclosure are applicable to any host cell.

In some embodiments, the present disclosure teaches a high-throughput (HTP) method of genomic engineering to evolve a microbe to acquire a desired phenotype, comprising: a) obtaining the genomes of an initial plurality of Saccharopolyspora microbes having perturbed genomes as an initial HTP genetic design Saccharopolyspora strain library, wherein the plurality of Saccharopolyspora microbes have the same genomic strain background, to thereby create an initial HTP genetic design and wherein the Saccharopolyspora strain library comprising comprises individual Saccharopolyspora strains with unique genetic variations; b) screening and selecting individual microbial strains of the initial HTP genetic design microbial strain library for the desired phenotype; c) providing a subsequent plurality of microbes that each comprise a unique combination of genetic variation, said genetic variation selected from the genetic variation present in at least two individual microbial strains screened in the preceding step, to thereby create a subsequent HTP genetic design microbial strain library; d) screening and selecting individual microbial strains of the subsequent HTP genetic design microbial strain library for the desired phenotype; e) repeating steps c)-d) one or more times, in a linear or non-linear fashion, until a microbe has acquired the desired phenotype, wherein each subsequent iteration creates a new HTP genetic design microbial strain library comprising individual microbial strains harboring unique genetic variations that are a combination of genetic variation selected from amongst at least two individual microbial strains of a preceding HTP genetic design microbial strain library.

When the genetic variations are combined, the function and/or identity of the genes that contain the genetic variations can be either considered, or not considered. In some embodiments, the function and/or identity of the genes that contain the genetic variations are not considered. For example, genetic variations of the same gene, or of genes having similar function/structure are selected for combination. In some embodiments, the function and/or identity of the genes that contain the genetic variations are not considered before the genetic variations are combined. In either case, the afterwards screening and selecting step can be carried out to identify engineered Saccharopolyspora strains having desired phenotype, such as improved production of a product of interest.

In some embodiments, the genetic variations are in one or more loci that relate to direct synthesis or metabolism of the product of interest, or loci that relate to regulation of the synthesis or the metabolism. In some embodiments, the genetic variations are in one or more loci that do not relate to direct synthesis or metabolism of the product of interest, and do not relate to regulation of the synthesis or the metabolism. In some embodiments, the genetic variations are randomly picked for the combination without any particular hypothesis of their functions or particular genome combination structure that are preferred. For example, in some embodiments, the purpose of the combination is not to substitute a DNA module in a genomic region that contains repeating segments of the DNA module, such as those in genes encoding a polyketide or a non-ribosomal peptide.

In some embodiments, in step (c) of the foregoing method in which genetic variations from different sources are combined, various techniques can be used. In some embodiments, a homologous recombination plasmid system is used. In some embodiments, Saccharopolyspora microbes that each comprises a unique combination of genetic variations in step (c) are produced by: 1) introducing a plasmid into an individual Saccharopolyspora strain belonging to the initial HTP genetic design Saccharopolyspora strain library, wherein the plasmid comprises (i) a selection marker, (ii) a counterselection marker, (iii) a DNA fragment having homology to the genomic locus of the base Saccharopolyspora strain, and plasmid backbone sequence, wherein the DNA fragment has a genetic variation derived from another individual Saccharopolyspora strain also belonging to the initial HTP genetic design Saccharopolyspora strain library; 2) selecting for Saccharopolyspora strains with integration event based on the presence of the selection marker in the genome; 3) selecting for Saccharopolyspora strains having the plasmid backbone looped out based on the absence of the counterselection marker gene.

In some embodiments, the methods of the disclosure are able to perform targeted genomic editing not only in these areas of genomic modularity, but enable targeted genomic editing across the genome, in any genomic context. Consequently, the targeted genomic editing of the disclosure can edit the S. spinosa genome in any region, and is not bound to merely editing in areas having modularity.

In some embodiments, the plasmid does not comprise a temperature sensitive.

In some embodiments, the selection step 3) is performed without replication of the integrated plasmid.

In some embodiments, the present disclosure teaches that the initial HTP genetic design microbial strain library is at least one selected from the group consisting of a promoter swap microbial strain library, SNP swap microbial strain library, start/stop codon microbial strain library, optimized sequence microbial strain library, a terminator swap microbial strain library, a transposon mutagenesis diversity library, a ribosomal binding site microbial strain library, an anti-metabolite selection/fermentation product resistance microbial library, or any combination thereof. In some embodiments, said microbial libraries are Saccharopolyspora spp. libraries.

In some embodiments, the present disclosure teaches methods of making a subsequent plurality of microbes that each comprise a unique combination of genetic variations, wherein each of the combined genetic variations is derived from the initial HTP genetic design microbial strain library or the HTP genetic design microbial strain library of the preceding step.

In some embodiments, the combination of genetic variations in the subsequent plurality of microbes will comprise a subset of all the possible combinations of the genetic variations in the initial HTP genetic design microbial strain library or the HTP genetic design microbial strain library of the preceding step.

In some embodiments, the present disclosure teaches that the subsequent HTP genetic design microbial strain library is a full combinatorial microbial strain library derived from the genetic variations in the initial HTP genetic design microbial strain library or the HTP genetic design microbial strain library of the preceding step.

For example, if the prior HTP genetic design microbial strain library only had genetic variations A, B, C, and D, then a partial combinatorial of said variations could include a subsequent HTP genetic design microbial strain library comprising three microbes each comprising either the AB, AC, or AD unique combinations of genetic variations (order in which the mutations are represented is unimportant). A full combinatorial microbial strain library derived from the genetic variations of the HTP genetic design library of the preceding step would include six microbes, each comprising either AB, AC, AD, BC, BD, or CD unique combinations of genetic variations.

In some embodiments, the methods of the present disclosure teach perturbing the genome utilizing at least one method selected from the group consisting of: random mutagenesis, targeted sequence insertions, targeted sequence deletions, targeted sequence replacements, transposon mutagenesis, or any combination thereof.

In some embodiments of the presently disclosed methods, the initial plurality of microbes comprise unique genetic variations derived from an industrial production strain microbe. In some embodiments, the microbes are Saccharopolyspora spp.

In some embodiments of the presently disclosed methods, the initial plurality of microbes comprise industrial production strain microbes denoted S1Gen1 and any number of subsequent microbial generations derived therefrom denoted SnGenn. In some embodiments, the microbes are Saccharopolyspora spp.

In some embodiments, the present disclosure teaches a method for generating a SNP swap microbial strain library, comprising the steps of: a) providing a reference microbial strain and a second microbial strain, wherein the second microbial strain comprises a plurality of identified genetic variations selected from single nucleotide polymorphisms, DNA insertions, and DNA deletions, which are not present in the reference microbial strain; b) perturbing the genome of either the reference microbial strain, or the second microbial strain, to thereby create an initial SNP swap microbial strain library comprising a plurality of individual microbial strains with unique genetic variations found within each strain of said plurality of individual microbial strains, wherein each of said unique genetic variations corresponds to a single genetic variation selected from the plurality of identified genetic variations between the reference microbial strain and the second microbial strain. In some embodiments, the microbial strains are Saccharopolyspora strains.

In some embodiments of SNP swap library, the genome of the reference microbial strain is perturbed to add one or more of the identified single nucleotide polymorphisms, DNA insertions, or DNA deletions, which are found in the second microbial strain.

In some embodiments of SNP swap library methods of the present disclosure, the genome of the second microbial strain is perturbed to remove one or more of the identified single nucleotide polymorphisms, DNA insertions, or DNA deletions, which are not found in the reference microbial strain.

In some embodiments, the genetic variations of the SNP swap library will comprise a subset of all the genetic variations identified between the reference microbial strain and the second microbial strain.

In some embodiments, the genetic variations of the SNP swap library will comprise all of the identified genetic variations identified between the reference microbial strain and the second microbial strain.

In some embodiments, the present disclosure teaches a method for rehabilitating and improving the phenotypic performance of an industrial microbial strain, comprising the steps of: a) providing a parental lineage microbial strain and an industrial microbial strain derived therefrom, wherein the industrial microbial strain comprises a plurality of identified genetic variations selected from single nucleotide polymorphisms, DNA insertions, and DNA deletions, not present in the parental lineage microbial strain; b) perturbing the genome of either the parental lineage microbial strain, or the industrial microbial strain, to thereby create an initial SNP swap microbial strain library comprising a plurality of individual microbial strains with unique genetic variations found within each strain of said plurality of individual microbial strains, wherein each of said unique genetic variations corresponds to a single genetic variation selected from the plurality of identified genetic variations between the parental lineage microbial strain and the industrial microbial strain; c) screening and selecting individual microbial strains of the initial SNP swap microbial strain library for phenotype performance improvements over a reference microbial strain, thereby identifying unique genetic variations that confer said microbial strains with phenotype performance improvements; d) providing a subsequent plurality of microbes that each comprise a unique combination of genetic variation, said genetic variation selected from the genetic variation present in at least two individual microbial strains screened in the preceding step, to thereby create a subsequent SNP swap microbial strain library; e) screening and selecting individual microbial strains of the subsequent SNP swap microbial strain library for phenotype performance improvements over the reference microbial strain, thereby identifying unique combinations of genetic variation that confer said microbial strains with additional phenotype performance improvements; and f) repeating steps d)-e) one or more times, in a linear or non-linear fashion, until a microbial strain exhibits a desired level of improved phenotype performance compared to the phenotype performance of the industrial microbial strain, wherein each subsequent iteration creates a new SNP swap microbial strain library comprising individual microbial strains harboring unique genetic variations that are a combination of genetic variation selected from amongst at least two individual microbial strains of a preceding SNP swap microbial strain library. In some embodiments, the microbial strains are Saccharopolyspora strains.

In some embodiments, the present disclosure teaches methods for rehabilitating and improving the phenotypic performance of an industrial microbial strain, wherein the genome of the parental lineage microbial strain is perturbed to add one or more of the identified single nucleotide polymorphisms, DNA insertions, or DNA deletions, which are found in the industrial microbial strain. In some embodiments, the microbial strains are Saccharopolyspora strains.

In some embodiments, the present disclosure teaches methods for rehabilitating and improving the phenotypic performance of an industrial microbial strain, wherein the genome of the industrial microbial strain is perturbed to remove one or more of the identified single nucleotide polymorphisms, DNA insertions, or DNA deletions, which are not found in the parental lineage microbial strain. In some embodiments, the microbial strains are Saccharopolyspora strains.

In some embodiments, the present disclosure teaches a method for generating a promoter swap microbial strain library, said method comprising the steps of: a) providing a plurality of target genes endogenous to a base microbial strain, and a promoter ladder, wherein said promoter ladder comprises a plurality of promoters exhibiting different expression profiles in the base microbial strain; b) engineering the genome of the base microbial strain, to thereby create an initial promoter swap microbial strain library comprising a plurality of individual microbial strains with unique genetic variations found within each strain of said plurality of individual microbial strains, wherein each of said unique genetic variations comprises one of the promoters from the promoter ladder operably linked to one of the target genes endogenous to the base microbial strain. In some embodiments, the microbial strains are Saccharopolyspora strains. In some embodiments, the promoter ladder comprises promoters having the sequences of SEQ ID No. 1 to SEQ ID No. 69, or combination thereof.

In some embodiments, the present disclosure teaches a promoter swap method of genomic engineering to evolve a microbe to acquire a desired phenotype, said method comprising the steps of: a) providing a plurality of target genes endogenous to a base microbial strain, and a promoter ladder, wherein said promoter ladder comprises a plurality of promoters exhibiting different expression profiles in the base microbial strain; b) engineering the genome of the base microbial strain, to thereby create an initial promoter swap microbial strain library comprising a plurality of individual microbial strains with unique genetic variations found within each strain of said plurality of individual microbial strains, wherein each of said unique genetic variations comprises one of the promoters from the promoter ladder operably linked to one of the target genes endogenous to the base microbial strain; c) screening and selecting individual microbial strains of the initial promoter swap microbial strain library for the desired phenotype; d) providing a subsequent plurality of microbes that each comprise a unique combination of genetic variation, said genetic variation selected from the genetic variation present in at least two individual microbial strains screened in the preceding step, to thereby create a subsequent promoter swap microbial strain library; e) screening and selecting individual microbial strains of the subsequent promoter swap microbial strain library for the desired phenotype; f) repeating steps d)-e) one or more times, in a linear or non-linear fashion, until a microbe has acquired the desired phenotype, wherein each subsequent iteration creates a new promoter swap microbial strain library comprising individual microbial strains harboring unique genetic variations that are a combination of genetic variation selected from amongst at least two individual microbial strains of a preceding promoter swap microbial strain library. In some embodiments, the microbial strains are Saccharopolyspora strains.

In some embodiments, the present disclosure teaches a method for generating a terminator swap microbial strain library, said method comprising the steps of: a) providing a plurality of target genes endogenous to a base microbial strain, and a terminator ladder, wherein said terminator ladder comprises a plurality of terminators exhibiting different expression profiles in the base microbial strain; b) engineering the genome of the base microbial strain, to thereby create an initial terminator swap microbial strain library comprising a plurality of individual microbial strains with unique genetic variations found within each strain of said plurality of individual microbial strains, wherein each of said unique genetic variations comprises one of the target genes endogenous to the base microbial strain operably linked to one or more of the terminators from the terminator ladder. In some embodiments, the microbial strains are Saccharopolyspora strains.

In some embodiments, the present disclosure teaches a terminator swap method of genomic engineering to evolve a microbe to acquire a desired phenotype, said method comprising the steps of: a) providing a plurality of target genes endogenous to a base microbial strain, and a terminator ladder, wherein said terminator ladder comprises a plurality of terminators exhibiting different expression profiles in the base microbial strain; b) engineering the genome of the base microbial strain, to thereby create an initial terminator swap microbial strain library comprising a plurality of individual microbial strains with unique genetic variations found within each strain of said plurality of individual microbial strains, wherein each of said unique genetic variations comprises one of the target genes endogenous to the base microbial strain operably linked to one or more of the terminators from the terminator ladder; c) screening and selecting individual microbial strains of the initial terminator swap microbial strain library for the desired phenotype; d) providing a subsequent plurality of microbes that each comprise a unique combination of genetic variation, said genetic variation selected from the genetic variation present in at least two individual microbial strains screened in the preceding step, to thereby create a subsequent terminator swap microbial strain library; e) screening and selecting individual microbial strains of the subsequent terminator swap microbial strain library for the desired phenotype; f) repeating steps d)-e) one or more times, in a linear or non-linear fashion, until a microbe has acquired the desired phenotype, wherein each subsequent iteration creates a new terminator swap microbial strain library comprising individual microbial strains harboring unique genetic variations that are a combination of genetic variation selected from amongst at least two individual microbial strains of a preceding terminator swap microbial strain library. In some embodiments, the microbial strains are Saccharopolyspora strains. In some embodiments, the terminator ladder comprises terminators having the sequences of SEQ ID No. 70 to SEQ ID No. 80, or combination thereof.

In some embodiments, the present disclosure teaches a transposon mutagenesis method of genomic engineering to evolve a microbe to acquire a desired phenotype, said method comprising the steps of: a) providing a transposase enzyme and a DNA payload sequence. In some embodiments, the transposase is functional in Saccharopolyspora spp. In some embodiments, the transpose is derived from EZ-Tn5 transposon system. In some embodiments, the DNA payload sequence is flanked by mosaic elements (ME) that can be recognized by said transposase. In some embodiments, the DNA payload can be a loss-of-function (LoF) transposon, or a gain-of-function (GoF) transposon. In some embodiments, the DNA payload comprises a selection marker. In some embodiments, the DNA payload comprises a counter-selection marker. In some embodiments, the counter-selection marker is used to facilitate loop-out of a DNA payload containing the selectable marker. In some embodiments, the GoF transposon comprises a GoF element. In some embodiments, the GoF transposon comprises a promoter sequence and/or a solubility tag sequence. In some embodiments, the methods further comprise b) combining the transpose and the DNA payload sequence to form a complex, and c) transforming the transpose-DNA payload complex to a microbial strain, thus resulting random integration of the DNA payload sequence in the genome of the microbial strain. Strains comprising the random integration of DNA payload form an initial transposon mutagenesis diversity library. In some embodiments, the methods further comprise d) screening and selecting individual microbial strains of the initial transposon mutagenesis diversity library for the desired phenotype. In some embodiments, the methods further comprise e) providing a subsequent plurality of microbes that each comprise a unique combination of genetic variation, said genetic variation selected from the genetic variation present in at least two individual microbial strains screened in the preceding step, to thereby create a subsequent transposon mutagenesis diversity library. In some embodiments, the methods further comprise f) screening and selecting individual microbial strains of the subsequent transposon mutagenesis diversity library for the desired phenotype. In some embodiments, the methods further comprise g) repeating steps e)-f) one or more times, in a linear or non-linear fashion, until a microbe has acquired the desired phenotype, wherein each subsequent iteration creates a new transposon mutagenesis diversity library comprising individual microbial strains harboring unique genetic variations that are a combination of genetic variation selected from amongst at least two individual microbial strains of a preceding transposon mutagenesis diversity library. In some embodiments, the microbial strains are Saccharopolyspora strains.

In some embodiments, the present disclosure teaches a method for generating a ribosomal binding site (RBS) swap microbial strain library. In some embodiments, said method comprises the steps of: a) providing a plurality of target genes endogenous to a base microbial strain, and a RBS ladder, wherein said RBS ladder comprises a plurality of ribosomal binding site exhibiting different expression profiles in the base microbial strain; b) engineering the genome of the base microbial strain, to thereby create an initial RBS microbial strain library comprising a plurality of individual microbial strains with unique genetic variations found within each strain of said plurality of individual microbial strains, wherein each of said unique genetic variations comprises one of the RBS from the RBS ladder operably linked to one of the target genes endogenous to the base microbial strain. In some embodiments, the microbial strains are Saccharopolyspora strains.

In some embodiments, the present disclosure teaches a ribosomal binding site (RBS) swap method of genomic engineering to evolve a microbe to acquire a desired phenotype, said method comprising the steps of: a) providing a plurality of target genes endogenous to a base microbial strain, and a RBS ladder, wherein said RBS ladder comprises a plurality of RBSs exhibiting different expression profiles in the base microbial strain; b) engineering the genome of the base microbial strain, to thereby create an initial RBS library comprising a plurality of individual microbial strains with unique genetic variations found within each strain of said plurality of individual microbial strains, wherein each of said unique genetic variations comprises one of the RBSs from the RBS ladder operably linked to one of the target genes endogenous to the base microbial strain; c) screening and selecting individual microbial strains of the initial RBS library for the desired phenotype; d) providing a subsequent plurality of microbes that each comprise a unique combination of genetic variation, said genetic variation selected from the genetic variation present in at least two individual microbial strains screened in the preceding step, to thereby create a subsequent RBS library; e) screening and selecting individual microbial strains of the subsequent RBS library for the desired phenotype; f) repeating steps d)-e) one or more times, in a linear or non-linear fashion, until a microbe has acquired the desired phenotype, wherein each subsequent iteration creates a new RBS library comprising individual microbial strains harboring unique genetic variations that are a combination of genetic variation selected from amongst at least two individual microbial strains of a preceding RBS library. In some embodiments, the microbial strains are Saccharopolyspora strains. In some embodiments, the terminator ladder comprises terminators having the sequences of SEQ ID No. 97 to SEQ ID No. 127, or combination thereof.

In some embodiments, the present disclosure teaches a method for generating an anti-metabolite/fermentation product resistance library. In some embodiments, the method comprises the steps of: a) providing a reference microbial strain and a second microbial strain, wherein the second microbial strain comprises a plurality of identifiable genetic variations, such genetic variations can be any type, including but not limited to single nucleotide polymorphisms, DNA insertions, and DNA deletions, which are not present in the reference microbial strain; and b) selecting for more resistant strains in the presence of one or more predetermined product produced by said microbes. In some embodiments, the method further comprises c) analyzing the performance of the selected strains (e.g., the yield of one or more product produced in the strains) and selecting strains having improved performance compared to the reference microbial strain by HTP screening. In some embodiments, the method further comprises d) identifying position and/or sequences of mutations causing the improved performance. These selected strains with confirmed improved performance form the initial anti-metabolite/fermentation product library. Such a library comprises a plurality of individual microbial strains with unique genetic variations found within each strain of said plurality of individual microbial strains, wherein each of said unique genetic variations corresponds to a single genetic variation selected from the plurality of identifiable genetic variations. In some embodiments, the microbial strains are Saccharopolyspora strains. In some embodiments, the predetermined product produced by the microbial strains is any molecule involved in the spinosyn synthesis pathway, or any molecule that can affect the production of spinosyn. In some embodiments, the predetermined products include, but are not limited to spinosyn A, spinosyn B, spinosyn C, spinosyn D, spinosyn E, spinosyn F, spinosyn G, spinosyn H, spinosyn I, spinosyn J, spinosyn K, spinosyn L, spinosyn M, spinosyn N, spinosyn O, spinosyn P, spinosyn Q, spinosyn R, spinosyn S, spinosyn T, spinosyn U, spinosyn V, spinosyn W, spinosyn X, spinosyn Y, norleucine, norvaline, pseudoaglycones (e.g., PSA, PSD, PSJ, PSL, etc., for the different spinosyn compounds), and alpha-Methyl-methionine (aMM).

In some embodiments, the present disclosure teaches iteratively improving the design of candidate microbial strains by (a) accessing a predictive model populated with a training set comprising (1) inputs representing genetic changes to one or more background microbial strains and (2) corresponding performance measures; (b) applying test inputs to the predictive model that represent genetic changes, the test inputs corresponding to candidate microbial strains incorporating those genetic changes; (c) predicting phenotypic performance of the candidate microbial strains based at least in part upon the predictive model; (d) selecting a first subset of the candidate microbial strains based at least in part upon their predicted performance; (e) obtaining measured phenotypic performance of the first subset of the candidate microbial strains; (f) obtaining a selection of a second subset of the candidate microbial strains based at least in part upon their measured phenotypic performance; (g) adding to the training set of the predictive model (1) inputs corresponding to the selected second subset of candidate microbial strains, along with (2) corresponding measured performance of the selected second subset of candidate microbial strains; and (h) repeating (b)-(g) until measured phenotypic performance of at least one candidate microbial strain satisfies a performance metric. In some cases, during a first application of test inputs to the predictive model, the genetic changes represented by the test inputs comprise genetic changes to the one or more background microbial strains; and during subsequent applications of test inputs, the genetic changes represented by the test inputs comprise genetic changes to candidate microbial strains within a previously selected second subset of candidate microbial strains. In some embodiments, the microbial strains are Saccharopolyspora strains.

In some embodiments, selection of the first subset may be based on epistatic effects. This may be achieved by: during a first selection of the first subset: determining degrees of dissimilarity between performance measures of the one or more background microbial strains in response to application of a plurality of respective inputs representing genetic changes to the one or more background microbial strains; and selecting for inclusion in the first subset at least two candidate microbial strains based at least in part upon the degrees of dissimilarity in the performance measures of the one or more background microbial strains in response to application of genetic changes incorporated into the at least two candidate microbial strains. In some embodiments, the microbial strains are Saccharopolyspora strains.

In some embodiments, the present invention teaches applying epistatic effects in the iterative improvement of candidate microbial strains, the method comprising: obtaining data representing measured performance in response to corresponding genetic changes made to at least one microbial background strain; obtaining a selection of at least two genetic changes based at least in part upon a degree of dissimilarity between the corresponding responsive performance measures of the at least two genetic changes, wherein the degree of dissimilarity relates to the degree to which the at least two genetic changes affect their corresponding responsive performance measures through different biological pathways; and designing genetic changes to a microbial background strain that include the selected genetic changes. In some cases, the microbial background strain for which the at least two selected genetic changes are designed is the same as the at least one microbial background strain for which data representing measured responsive performance was obtained. In some embodiments, the microbial strains are Saccharopolyspora strains.

In some embodiments, the present disclosure teaches HTP strain improvement methods utilizing only a single type of genetic microbial library. For example, in some embodiments, the present disclosure teaches HTP strain improvement methods utilizing only SNP swap libraries. In other embodiments, the present disclosure teaches HTP strain improvement methods utilizing only PRO swap libraries. In some embodiments, the present disclosure teaches HTP strain improvement methods utilizing only STOP swap libraries. In some embodiments, the present disclosure teaches HTP strain improvement methods utilizing only Start/Stop Codon swap libraries. In some embodiments, the present disclosure teaches HTP strain improvement methods utilizing only a transposon mutagenesis diversity library. In some embodiments, the present disclosure teaches HTP strain improvement methods utilizing only a ribosomal binding site microbial strain library. In some embodiments, the present disclosure teaches HTP strain improvement methods utilizing only an anti-metabolite selection/fermentation product resistance microbial library. In some embodiments, the microbial strains are Saccharopolyspora strains.

In other embodiments, the present disclosure teaches HTP strain improvement methods utilizing two or more types of genetic microbial libraries. For example, in some embodiments, the present disclosure teaches HTP strain improvement methods combining SNP swap and PRO swap libraries. In some embodiments, the present disclosure teaches HTP strain improvement methods combining SNP swap and STOP swap libraries. In some embodiments, the present disclosure teaches HTP strain improvement methods combining PRO swap and STOP swap libraries. In some embodiments, the present disclosure teaches HTP strain improvement methods combining SNP swap library with a transposon mutagenesis diversity library, a ribosomal binding site microbial strain library, and/or an anti-metabolite selection/fermentation product resistance microbial library. In some embodiments, the present disclosure teaches HTP strain improvement methods combining PRO swap library with a transposon mutagenesis diversity library, a ribosomal binding site microbial strain library, and/or an anti-metabolite selection/fermentation product resistance microbial library. In some embodiments, the present disclosure teaches HTP strain improvement methods combining STOP swap library with a transposon mutagenesis diversity library, a ribosomal binding site microbial strain library, and/or an anti-metabolite selection/fermentation product resistance microbial library. In some embodiments, the present disclosure teaches HTP strain improvement methods combining terminator swap library with a transposon mutagenesis diversity library, a ribosomal binding site microbial strain library, and/or an anti-metabolite selection/fermentation product resistance microbial library. In some embodiments, the present disclosure teaches HTP strain improvement methods combining a transposon mutagenesis diversity library with a ribosomal binding site microbial strain library, and/or an anti-metabolite selection/fermentation product resistance microbial library. In some embodiments, the present disclosure teaches HTP strain improvement methods combining a ribosomal binding site microbial strain library, and an anti-metabolite selection/fermentation product resistance microbial library.

In other embodiments, the present disclosure teaches HTP strain improvement methods utilizing multiple types of genetic microbial libraries. In some embodiments, the genetic microbial libraries are combined to produce combination mutations (e.g., promoter/terminator combination ladders applied to one or more genes). In yet other embodiments, the HTP strain improvement methods of the present disclosure can be combined with one or more traditional strain improvement methods.

In some embodiments, the HTP strain improvement methods of the present disclosure result in an improved host cell. That is, the present disclosure teaches methods of improving one or more host cell properties. In some embodiments the improved host cell property is selected from the group consisting of volumetric productivity, specific productivity, yield or titre, of a product of interest produced by the host cell. In some embodiments the improved host cell property is volumetric productivity. In some embodiments the improved host cell property is specific productivity. In some embodiments the improved host cell property is yield.

In some embodiments, the HTP strain improvement methods of the present disclosure result in a host cell that exhibits a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300% or more of an improvement in at least one host cell property over a control host cell that is not subjected to the HTP strain improvements methods (e.g., an X % improvement in yield or productivity of a biomolecule of interest, incorporating any ranges and subranges therebetween). In some embodiments, the HTP strain improvement methods of the present disclosure are selected from the group consisting of SNP swap, PRO swap, STOP swap, a transposon mutagenesis diversity library, a ribosomal binding site microbial strain library, an anti-metabolite selection/fermentation product resistance microbial library, and combinations thereof.

Thus, in some embodiments, the SNP swap methods of the present disclosure result in a host cell that exhibits a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300% or more of an improvement in at least one host cell property over a control host cell that is not subjected to the SNP swap methods (e.g., an X % improvement in yield or productivity of a biomolecule of interest, incorporating any ranges and subranges therebetween).

Thus, in some embodiments, the PRO swap methods of the present disclosure result in a host cell that exhibits a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300% or more of an improvement in at least one host cell property over a control host cell that is not subjected to the PRO swap methods (e.g., an X % improvement in yield or productivity of a biomolecule of interest, incorporating any ranges and subranges therebetween).

In some embodiments, the terminator swap methods of the present disclosure result in a host cell that exhibits a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300% or more of an improvement in at least one host cell property over a control host cell that is not subjected to the PRO swap methods (e.g., an X % improvement in yield or productivity of a biomolecule of interest, incorporating any ranges and subranges therebetween).

In some embodiments, the transposon mutagenesis methods of the present disclosure result in a host cell that exhibits a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300% or more of an improvement in at least one host cell property over a control host cell that is not subjected to the PRO swap methods (e.g., an X % improvement in yield or productivity of a biomolecule of interest, incorporating any ranges and subranges therebetween).

In some embodiments, the methods of using ribosomal binding site library of the present disclosure result in a host cell that exhibits a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300% or more of an improvement in at least one host cell property over a control host cell that is not subjected to the PRO swap methods (e.g., an X % improvement in yield or productivity of a biomolecule of interest, incorporating any ranges and subranges therebetween). In some embodiments, the anti-metabolite selection/fermentation product resistance methods of the present disclosure result in a host cell that exhibits a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200%, 250%, 300% or more of an improvement in at least one host cell property over a control host cell that is not subjected to the PRO swap methods (e.g., an X % improvement in yield or productivity of a biomolecule of interest, incorporating any ranges and subranges therebetween).

The present disclosure also provides a method for rapid consolidation of genetic changes in two or more microbial strains and for generating genetic diversity in Saccharopolyspora spp. In some embodiments, the method is based on protoplast fusion. In some embodiments, when at least one of the microbial strains contains a “marked” mutation, the method comprises the following steps: (1) choosing parent strains from a pool of engineered strains for consolidation; (2) preparing protoplasts (e.g., removing the cell wall, etc.) from the strains that are to be consolidated; and (3) fusing the strains of interest; (4) recovering of cells. (5) selecting cells which carry the “marked” mutation, and (6) genotyping growing cells for the presence of mutations coming for the other parent strains. Optionally, the method further comprises the step of (7) removing the plasmid form the “marked” mutation. In some embodiments, when none of the microbial strains contains a “marked” mutation, the method comprises the following steps: (1) choosing parent strains from a pool of engineered strains for consolidation; (2) preparing protoplasts (e.g., removing the cell wall, etc.) from the strains that are to be consolidated; and (3) fusing the strains of interest; (4) recovering of cells. (5) selecting cells for the presence of mutations coming from the first parent strain, and (6) selecting cells for the presence of mutations coming for the other parent strains. In some embodiments, the strains are selected based on a phenotype associated with the mutation coming from the first parent strain and/or from the other parent strain. In some embodiments, the strains are selected based on genotyping. In some embodiments, the genotyping step is done in a high-throughput procedure.

In some embodiments, in step (3), to increase the odds of generating useful (novel) combinations of mutants, fewer cells of the stain with “marked” mutation can be used, thus increasing the chances that these “marked” cells would have interacted and fused with cells carrying different mutations. In some embodiments, in step (4), cells are plated on osmotically stabilized media without the use of agar overlay, which simplifies the procedure and allows for easier automation. The osmo-stabilizers are such that allow for the growth of cells which might contain the counter-selection marker gene (e.g., sacB gene). Protoplasted cells are very sensitive to treatment and are easy to kill. This step ensures that enough cells are recovered. The better this step works, the more material can be used for downstream analysis. In some embodiments, in step (5), the step is accomplished by overlaying appropriate antibiotic onto the growing cells. In case neither of the parent cell carries a “marked” mutation, the strains can be genotyped by other means to identify strains of interest. This step could be optional but it ensures that cells that have most likely undergone cell fusion are enriched. It is possible to “mark” multiple loci and this way one can generate the combinations of interest faster, but then multiple plasmids may have to be removed if one would like to have “scarless” strains. In some embodiments, in step (6), the number of colonies to genotype depends on the complexity of the cross as well as the selection scheme. In some embodiments, step (7) is optional and is recommended for additional verification or client delivery. In some embodiments, at the end of engineering cycles for a strain, all plasmid remnants need to be removed. When and how often this is carried out is at the discretion of the user. In some embodiments, the presence of the counter-selectable sacB gene makes this step straightforward. In some embodiments, at least one of the stains has a “marked” mutation. In some embodiments, the number of strains fused during a single consolidation step can be two or more, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, 500, or more. In some embodiments, one or more of the strain for fusing can be tagged by a selection marker at loci of interest.

The present disclosure also provides reporter proteins and related assays for use in Saccharopolyspora spp. In some embodiments, the reporter proteins are selected from group consisting of Dasher GFP (SEQ ID No. 81), Paprika RFP (SEQ ID No. 82), and enzyme beta-glucuronidase (gusA) (SEQ ID No. 83). In some embodiments, nucleotide sequences encoding these reporter genes are codon optimized for either E. coli or Saccharopolyspora spp. In some embodiments, the florescent proteins of the present disclosure have spectra that did not overlap with the spectrum of endogenous florescence observed in Saccharopolyspora spp. In some embodiments, the reporter proteins are used to determine activity of a gene of interest in Saccharopolyspora spp. In some embodiments, the reporter proteins are used to determine the strength of a promoter sequence of interest in Saccharopolyspora spp. Such a promoter can be natural, synthetic, or combinations thereof. The natural promoter can be either native to Saccharopolyspora spp., or heterologous to Saccharopolyspora spp.

In some embodiments, the reporter proteins are used to determine the strength of a terminator sequence of interest in Saccharopolyspora spp. In some embodiments, the reporter proteins are used to determine the strength of a start codon or a stop codon of interest in Saccharopolyspora spp. In some embodiments, the reporter proteins are used to determine the strength of a ribosomal binding site sequence of interest in Saccharopolyspora spp. In some embodiments, the reporter proteins are used to as a marker to determine if a sequence has been looped out from the genome of Saccharopolyspora spp.

The present disclosure also provides neutral integration sites (NISs) for the insertion of genetic elements in Saccharopolyspora spp. These neutral integration sites are genetic loci into which individual genes or multi-gene cassettes can be stably and efficiently integrated within the genome of Saccharopolyspora spp. strains. Integration of sequences into these sites have no or limited effect on growth of the strains. In some embodiments, the neutral integration sites are selected from the group consisting of loci having sequences of SEQ ID No. 132 to SEQ ID No. 142. In some embodiments, unique genetic sequences (i.e., watermarks) can be inserted in the NIS to label a strain or lineage (e.g., for proprietary reasons).

In some embodiments, one or more genetic elements are inserted into a single neutral integration site described herein of Saccharopolyspora spp. In some embodiments, one or more genetic elements are inserted into two or more neutral integration sites described herein of Saccharopolyspora spp., such as 2, 3, 4, 5, 6, 7, 8, 9, 10, or 11 of the neutral integration sites. In some embodiments, Saccharopolyspora spp. strains having genetic element(s) inserted into the neutral integration site(s) have comparable growth compared to a reference strain that does not have the insertion. In some embodiments, Saccharopolyspora spp. strains having genetic element(s) inserted into the neutral integration site(s) have improved performance (e.g., improved yield of one or more molecules of interest, such as a spinosyn) compared to a reference strain that does not have the insertion. In some embodiments, Saccharopolyspora spp. strains having genetic element(s) inserted into the neutral integration site(s) form a diversity library, which can be further combined with other strain libraries described in the present disclosure to create and select for new strains having improved performance compared to a reference strain. In some embodiments, Saccharopolyspora spp. strains having genetic element(s) inserted into the neutral integration site(s) can be further mutagenized and selected for additional, new strains having desired phenotypes.

The present disclosure also provides methods for transferring genetic material from donor microorganism cells to recipient cells of a Saccharopolyspora microorganism. In some embodiments, wherein the method comprises the steps of: (1) subculturing recipient cells to mid-exponential phase (optional); (2) subculturing donor cells to mid-exponential phase (optional); (3) combining donor and recipient cells; (4) plating donor and recipient cell mixture on a conjugation media; (5) incubating plates to allow cells to conjugate; (6) applying antibiotic selection against donor cells; (7) applying antibiotic selection against non-integrated recipient cells; and (8) further incubating plates to allow for the outgrowth of integrated recipient cells. In some embodiments, the donor microorganism cells are E. coli cells. In some embodiments, the recipient microorganism cells are Saccharopolyspora sp. cells, such as Saccharopolyspora spinosa.

In some embodiments, at least two, three, four, five, six, seven or more of the following conditions are utilized: (1) recipient cells are washed; (2) donor cells and recipient cells are conjugated at a temperature of about 30° C.; (3) recipient cells are sub-cultured for at least about 48 hours before conjugating; (4) the ratio of donor cells:recipient cells for conjugation is about 1:0.6 to 1:1.0; (5) an antibiotic drug for selection against the donor cells is delivered to the mixture about 15 to 24 hours after the donor cells and the recipient cells are mixed; (6) an antibiotic drug for selection against the recipient cells is delivered to the mixture about 40 to 48 hours after the donor cells and the recipient cells are mixed; (7) the conjugation media plated with donor and recipient cell mixture is dried for at least about 3 hours to 10 hours; (8) the conjugation media comprises at least about 3 g/L glucose; (9) the concentration of donor cells is about OD600=0.4; and (10) the concentration of recipient cells is about OD540=13.0.

In some embodiments, the antibiotic drug for selection against the donor cells is a drug that the donor cells are sensitive to, while the recipient cells are resistant to. In some embodiments, the antibiotic drug for selection against the recipient cells is a drug that the donor cells are resistant to, while the recipient cells are sensitive to.

In some embodiments, the antibiotic drug for selection against the donor cells is nalidixic, and the concentration is about 50 to about 150 μg/ml. In some embodiments, the antibiotic drug for selection against the donor cells is spectinomycin, and the concentration is about 10 to about 300 μg/ml.

In some embodiments, the antibiotic drug for selection against the donor cells is nalidixic, and the concentration is about 100 μg/ml.

In some embodiments, the antibiotic drug for selection against the recipient cells is apramycin, and the concentration is about 50 to about 250 μg/ml.

In some embodiments, the antibiotic drug for selection against the recipient cells is apramycin, and the concentration is about 100 μg/ml.

In some embodiments, the method is performed in a high-throughput process. In some embodiments, the method is performed on a 48-well Q-trays.

In some embodiments, the high-throughput process is automated.

In some embodiments, the mixture of donor cells and recipient cells is a liquid mixture, and ample volume of the liquid mixture is plated on the medium with a rocking motion, wherein the liquid mixture is dispersed over the whole area of the medium.

In some embodiments, the method comprises automated process of transferring exconjugants by colony picking with yeast pins for subsequent inoculation of recipient cells with integrated DNA provided by the donor cells.

In some embodiments, the colony picking is performed in either a dipping motion, or a stirring motion.

In some embodiments, the conjugating media is a modified ISP4 media comprising about 3-10 g/L glucose.

In some embodiments, the total number of donor cells or recipient cells in the mixture is about 5×10⁶ to about 9×10⁶. In some embodiments, concentration of the donor cells used for conjugation is about OD 0.1 to about OD 0.6.

In some embodiments, the method is performed with at least two, three, four, five, six, or seven of the following conditions: (1) recipient cells are washed before conjugating; (2) donor cells and recipient cells are conjugated at a temperature of about 30° C.; (3) recipient cells are sub-cultured for at least about 48 hours before conjugating; (4) the ratio of donor cells:recipient cells for conjugation is about 1:0.8; (5) an antibiotic drug for selection against the donor cells is delivered to the mixture about 20 hours after the donor cells and the recipient cells are mixed; (6) the amount of the donor cells or the amount of the recipient cells in the mixture is about 7×10⁶, and (7) the conjugation media comprises about 6 g/L glucose

The present disclosure also provides methods of targeted genomic editing in a Saccharopolyspora strain, resulting in a scarless Saccharopolyspora strain containing a genetic variation at a targeted genomic locus. In some embodiments, the methods comprises a) introducing a plasmid into a Saccharopolyspora strain, said plasmid comprising: (i) a selection marker, (ii) a counterselection marker, (iii) a DNA fragment containing a genetic variation to be integrated into the Saccharopolyspora genome at a target locus, said DNA fragment having homology arms to the target genomic locus flanking the desired genetic variation, and (iv) plasmid backbone sequence.

In some embodiments, the methods of targeted genomic editing in a Saccharopolyspora strain further comprises b) selecting for a Saccharopolyspora strain that has undergone an initial homologous recombination and has the genetic variation integrated into the target locus based on the presence of the selection marker in the genome; and c) selecting for a Saccharopolyspora strain that has the genetic variation integrated into the target locus, but has undergone an additional homologous recombination that loops-out the plasmid backbone, based on the absence of the counterselection marker. In some embodiments, the selection step b) and the selection step c) are performed simultaneously. In some embodiments, the selection step b) and the selection step c) are performed sequentially. As a result of the selections, the DNA fragment containing a genetic variation is integrated into the Saccharopolyspora genome at the target locus of selected Saccharopolyspora strains, while the selection marker, the counter-selection marker, and/or the plasmid backbone sequence are “looped-out” from the genome of the selected Saccharopolyspora strains.

The targeted genomic locus may comprise any region of the Saccharopolyspora genome. In some embodiments, the targeted genomic locus comprises a genomic region that does not contain repeating segments of encoding DNA modules.

In some embodiments, the plasmid for targeted genomic editing does not comprise a temperature sensitive replicon.

In some embodiments, the plasmid for targeted genomic editing does not comprise an origin of replication.

In some embodiments, the selection step (c) is performed without replication of the integrated plasmid.

In some embodiments, the plasmid is a single homologous recombination vector. In some embodiments, the plasmid is a double homologous recombination vector.

In some embodiments, the counterselection marker is a sacB gene or a pheS gene.

In some embodiments, the sacB gene or pheS gene is codon-optimized for Saccharopolyspora spinosa.

In some embodiments, the sacB gene comprises the sequence of SEQ ID NO. 146. In some embodiments, the pheS gene comprises the sequence of SEQ ID NO. 147 or SEQ ID NO. 148.

In some embodiments, the plasmid is introduced into the Saccharopolyspora strain by transformation.

In some embodiments, the transformation is a protoplast transformation.

In some embodiments, the plasmid is introduced into the Saccharopolyspora strain by conjugation, wherein the Saccharopolyspora strain is a recipient cell, and a donor cell comprising the plasmid transfers the plasmid to the Saccharopolyspora strain. In some embodiments, the conjugation is based on an E. coli donor cell comprising the plasmid. In some embodiments, the target locus is a locus associated with production of a compound of interest in the Saccharopolyspora strain. In some embodiments, the compound of interest is a spinosyn.

The resulting Saccharopolyspora strain has edited genome may have one or more desired traits, such as improved production of a compound of interest. In some embodiments, the resulting Saccharopolyspora strain has increased production of a compound of interest compared to a control strain without the genomic editing.

In some embodiments, the method is performed as a high-throughput procedure.

The foregoing high-throughput (HTP) methods can involve the utilization of at least one piece of automated equipment (e.g. a liquid handler or plate handler machine) to carry out at least one step of said method. The HTP methods of the present disclosure provide a faster and less labor-intensive way of genomic engineering of a microbe (e.g., a Saccharopolyspora species), as the methods can be carried out in a large scale with less human resource. For example, in some embodiments, any method of the present disclosure is performed on a 48-well plate, a 96-well plate, a 192 well plate, a 384-well plate, etc., so that multiple strains are created and/or tested simultaneously, rather than one by one. The methods save a lot of time compared to other methods in which no automated equipment is used. In some embodiments, the methods are about 10 times, 20 times, 30 times, 40 times, 50 ties, 60 times, 70 times, 80 times, 90 times, 100 times, 150 times, 200 times, 250 times, 300 times or more faster compared to other methods in which no automated equipment is used, when the same or less human resource is used in the methods of the present disclosure.

### BRIEF DESCRIPTION OF THE FIGURES

FIG. 1 depicts a DNA recombination method of the present disclosure for increasing variation in diversity pools. DNA sections, such as genome regions from related species, can be cut via physical or enzymatic/chemical means. The cut DNA regions are melted and allowed to reanneal, such that overlapping genetic regions prime polymerase extension reactions. Subsequent melting/extension reactions are carried out until products are reassembled into chimeric DNA, comprising elements from one or more starting sequences.

FIG. 2 outlines methods of the present disclosure for generating new host organisms with selected sequence modifications (e.g., 100 SNPs to swap). Briefly, the method comprises (1) desired DNA inserts are designed and generated by combining one or more synthesized oligos in an assembly reaction, (2) DNA inserts are cloned into transformation plasmids, (3) completed plasmids are transferred into desired production strains, where they are integrated into the host strain genome, and (4) selection markers and other unwanted DNA elements are looped out of the host strain. Each DNA assembly step may involve additional quality control (QC) steps, such as cloning plasmids into E. coli bacteria for amplification and sequencing.

FIG. 3 depicts assembly of transformation plasmids of the present disclosure, and their integration into host organisms. The insert DNA is generated by combining one or more synthesized oligos in an assembly reaction. DNA inserts containing the desired sequence are flanked by regions of DNA homologous to the targeted region of the genome. These homologous regions facilitate genomic integration, and, once integrated, form direct repeat regions designed for looping out vector backbone DNA in subsequent steps. Assembled plasmids contain the insert DNA, and optionally, one or more selection markers.

FIG. 4 depicts procedure for looping-out selected regions of DNA from host strains. Direct repeat regions of the inserted DNA and host genome can “loop out” in a recombination event. Cells counter selected for the selection marker contain deletions of the loop DNA flanked by the direct repeat regions.

FIG. 5 depicts an embodiment of the strain improvement process of the present disclosure. Host strain sequences containing genetic modifications (Genetic Design) are tested for strain performance improvements in various strain backgrounds (Strain Build). Strains exhibiting beneficial mutations are analyzed (Hit ID and Analysis) and the data is stored in libraries for further analysis (e.g., SNP swap libraries, PRO swap libraries, and combinations thereof, among others). Selection rules of the present disclosure generate new proposed host strain sequences based on the predicted effect of combining elements from one or more libraries for additional iterative analysis.

FIG. 6A to FIG. 6B depicts the DNA assembly, transformation, and strain screening steps of one of the embodiments of the present disclosure. FIG. 6A depicts the steps for building DNA fragments, cloning said DNA fragments into vectors, transforming said vectors into host strains, and looping out selection sequences through counter selection. FIG. 6B depicts the steps for high-throughput culturing, screening, and evaluation of selected host strains. This figure also depicts the optional steps of culturing, screening, and evaluating selected strains in culture tanks.

FIG. 7 depicts one embodiment of the automated system of the present disclosure. The present disclosure teaches use of automated robotic systems with various modules capable of cloning, transforming, culturing, screening and/or sequencing host organisms.

FIG. 8 depicts an overview of an embodiment of the host strain improvement program of the present disclosure.

FIG. 9 is a representation of the genome of Saccharopolyspora spinosa, comprising around 8.4 million base pairs (adopted from Galm and Sparks, “Natural product derived insecticides: discovery and development of spinetoram” J. Ind Microbiol Biotechnol. 2015, DOI 10.1007/s10295-015-1710-x), which is incorporated by reference in its entirety for all purposes.

FIG. 10 depicts a transformation experiment of the present disclosure in Corynebacterium. DNA inserts ranging from 0.5 kb to 5.0 kb are targeted for insertion into various regions (shown as relative positions 1-24) of the genome of a microbial strain. Light color indicates successful integration, while darker color indicates insertion failure.

FIG. 11 depicts a first-round SNP swapping experiment according to the methods of the present disclosure. (1) all the SNPs from C will be individually and/or combinatorially cloned into the base A strain (“wave up” A to C). (2) all the SNPs from C will be individually and/or combinatorially removed from the commercial strain C (“wave down” C to A). (3) all the SNPs from B will be individually and/or combinatorially cloned into the base A strain (wave up A to B). (4) all the SNPs from B will be individually and/or combinatorially removed from the commercial strain B (wave down B to A). (5) all the SNPs unique to C will be individually and/or combinatorially cloned into the commercial B strain (wave up B to C). (6) all the SNPs unique to C will be individually and/or combinatorially removed from the commercial strain C (wave down C to B).

FIG. 12A to FIG. 12D illustrate example gene targets involved in spinosyn synthesis, which can be utilized in a promoter swap process. FIG. 12A is a graphic representation of the spinosyn biosynthetic gene cluster including genes that reside at other genomic loci. FIG. 12B is the biosynthetic assembly of the spinosyn polyketide scaffold.

FIG. 12C represents cross-linking and tailoring reactions to form the final spinosyn A and D molecules. FIG. 12D represents fermentation-based production of spinosyn J with subsequent synthetic conversion into spinetoram via 3′-O-ethylation and 5,6-double bond reduction. All figures are adopted from Galm and Sparks, 2015.

FIG. 13 illustrates an exemplary promoter library that is being utilized to conduct a promoter swap process for the identified gene targets. Promoters utilized in the PRO swap (i.e. promoter swap) process are those found in Example 4 and Table 1. Non-limiting examples of pathway targets are depicted in the left box and the varying expression strength of members of the promoter ladder are depicted in the middle box. As one can see, the promoters provide a “ladder” of expression strength that ranges from strong to weak.

FIG. 14 illustrates that promoter swapping genetic outcomes depend on the particular gene being targeted.

FIG. 15 depicts exemplary HTP promoter swapping data showing average fluorescence of promoter strains grown for 48 hours in seed media (non-production conditions_presented as fold change relative to PermE*, a non-native promoter previously characterized in S. spinosa. The relative strengths span an approximate 50-fold dynamic range. Three native promoters are among the five strongest promoters in the ladder and P1 is approximately 5-fold stronger than PermE* and ˜2× stronger than the next strongest promoter. Also, the relative strengths of the synthetic promoters is similar to results reported in the literature for Streptomyces. A and B represent different strains of S. spinosa. The X-axis represents different promoters, and the Y-axis includes relative strength of each promoter as measured by fluorescence. The taught PRO swap molecular tool can be utilized to optimize and/or increase the production of any compound of interest. One of skill in the art would understand how to choose target genes, encoding the production of a desired compound, and then utilize the taught PRO swap procedure. One of skill in the art would readily appreciate that the demonstrated data exemplifying lysine yield increases taught herein, along with the detailed disclosure presented in the application, enables the PRO swap molecular tool to be a widely applicable advancement in HTP genomic engineering.

FIG. 16 is a summary of log-transformed normalized fluorescence measured in promoter ladder strains (Strain A and Strain B) grown in Zymergen's 96-well plate model (production-relevant conditions). These strains have different promoter>GFP expression cassettes integrated in the host genome. Shaded boxes indicate strains that were evaluated during the first rounds of promoter evaluation and represented internal controls in later experiments. The lower bar indicates the average fluorescence baseline.

FIG. 17 depicts improved spinosyn J+L titer in strains engineered with promoters P21 and P1 described in Table 8. Particularly, 7000225635 contains P1 promoter in strain_B_3 g05097; 7000206640contains P21 promoter in strain_B_3 g00920; 7000206509 contains P1 promoter in strain_B_3 g02509; 7000206745 contains P21 promoter in strain_B_3 g07456; 7000206752 contains P21 promoter in strain_B_3 g07766; and 7000235481 contains P21 promoter in strain_B_3 g04679. Each strain ID represents a promoter swap at a given gene (with the genotypes represented above), and therefore each strain ID refers to a specific strain genotype. Each dot represents a well or sample of that strain tested in our high-throughput assay (i.e., they are all individual data points collected on the same strain). Selected promoter swap strains showed improvement over parent strain (700153593) when tested in high-throughput assay for spinosyn production. Strains were engineered by using conjugation to introduce a plasmid containing a selectable marker, the promoter-gene pair, and homology regions to integrate into the genome at a neutral site (see counterselectable marker section in the present disclosure for more details on the method).

FIG. 18 illustrates an example of the distribution of relative strain performances for the input data under consideration done in Coynebacterium by using the method described in the present disclosure. However, similar procedures have been customized for Saccharopolyspora and are being successfully carried out by the inventors. A relative performance of zero indicates that the engineered strain performed equally well to the in-plate base strain. The processes described herein are designed to identify the strains that are likely to perform significantly above zero.

FIG. 19 depicts the DNA assembly and transformation steps of one of the embodiments of the present disclosure. The flow chart depicts the steps for building DNA fragments, cloning said DNA fragments into vectors, transforming said vectors into host strains, and looping out selection sequences through counter selection.

FIG. 20 depicts the steps for high-throughput culturing, screening, and evaluation of selected host strains. This figure also depicts the optional steps of culturing, screening, and evaluating selected strains in culture tanks.

FIG. 21 depicts expression profiles of illustrative promoters exhibiting a range of regulatory expression, according to the promoter ladders of the present disclosure. Promoter A expression peaks at the lag phase of bacterial cultures, while promoter B and C peak at the exponential and stationary phase, respectively.

FIG. 22 depicts expression profiles of illustrative promoters exhibiting a range of regulatory expression, according to the promoter ladders of the present disclosure. Promoter A expression peaks immediately upon addition of a selected substrate, but quickly returns to undetectable levels as the concentration of the substrate is reduced. Promoter B expression peaks immediately upon addition of the selected substrate and lowers slowly back to undetectable levels together with the corresponding reduction in substrate. Promoter C expression peaks upon addition of the selected substrate, and remains highly expressed throughout the culture, even after the substrate has dissipated.

FIG. 23 depicts expression profiles of illustrative promoters exhibiting a range of constitutive expression levels, according to the promoter ladders of the present disclosure. Promoter A exhibits the lowest expression, followed by increasing expression levels promoter B and C, respectively.

FIG. 24 diagrams an embodiment of LIMS system of the present disclosure for strain improvement.

FIG. 25 diagrams a cloud computing implementation of embodiments of the LIMS system of the present disclosure.

FIG. 26 depicts an embodiment of the iterative predictive strain design workflow of the present disclosure.

FIG. 27 diagrams an embodiment of a computer system, according to embodiments of the present disclosure.

FIG. 28 depicts the workflow associated with the DNA assembly according to one embodiment of the present disclosure. This process is divided up into 4 stages: parts generation, plasmid assembly, plasmid QC, and plasmid preparation for transformation. During parts generation, oligos designed by Laboratory Information Management System (LIMS) are ordered from an oligo sequencing vendor and used to amplify the target sequences from the host organism via PCR. These PCR parts are cleaned to remove contaminants and assessed for success by fragment analysis, in silico quality control comparison of observed to theoretical fragment sizes, and DNA quantification. The parts are transformed into yeast along with an assembly vector and assembled into plasmids via homologous recombination. Assembled plasmids are isolated from yeast and transformed into E. coli for subsequent assembly quality control and amplification. During plasmid assembly quality control, several replicates of each plasmid are isolated, amplified using Rolling Circle Amplification (RCA), and assessed for correct assembly by enzymatic digest and fragment analysis. Correctly assembled plasmids identified during the QC process are hit picked to generate permanent stocks and the plasmid DNA extracted and quantified prior to transformation into the target host organism.

FIG. 29 is a flowchart illustrating the consideration of epistatic effects in the selection of mutations for the design of a microbial strain, according to embodiments of the disclosure.

FIG. 30 illustrates an example of the protocol for consolidating two Saccharopolyspora spp. strains through protoplast fusion.

FIG. 31A to FIG. 31D shows schematic of dasherGFP and paprikaRFP fluorescence spectra (FIG. 31A and FIG. 31B, respectively) and relative fluorescence of a mixed (1:1) culture of GFP and RFP strains (FIG. 31C and FIG. 31D, respectively). The fluorescent excitation and emission spectra of dasherGFP is distinct from paprikaRFP, enabling GFP or RFP fluorescence to be measured from a sample expressing both reporter (bottom panels, Mix (1:1)) without significant interference from the other reporter. Bottom Left: relative GFP fluorescence of an ermE*>RFP, ermE*>GFP strain and a 1:1 mix of both strains. In the RFP strain there is little to no detectable fluorescence in the GFP channel relative to that measured from the ermE*>GFP strain and the mixed culture produces a signal that is (as expected) approximately ½ the GFP strain alone. Bottom Right: similarly, when the optimal parameters for RFP fluorescence are used (top right) a strong fluorescence signal is detected for the ermE*>RFP strain, but little to no signal is observed for the ermE*>GFP strain and the 1:1 mix, again, produces a fluorescent signal that is approximately ½ that of the ermE*>RFP strain. Thus, the fluorescent reporters DasherGFP and PaprikaRFP work in S. spinosa and have distinct fluorescence signatures. The fluorescent excitation and emission spectra of DasherGFP is distinct from PaprikaRFP, enabling GFP or RFP fluorescence to be measured from a sample expressing both reporter (bottom panels, Mix (1:1)) without significant interference from the other reporter.

FIG. 32 shows schematic depicting the design of the bi-cistronic, dual reporter test cassette and relative fluorescence expected for a functional transcription terminator and the no-terminator (NoT) control. The terminator test cassette consists of a two fluorescent, reporter proteins—dasherGFP (GFP) and paprikaRFP (RFP)—arranged in tandem. Bi-cistronic expression of these reporters is driven by the ermE* promoter. Expression of the downstream reporter (RFP) is enabled by the upstream ribosomal binding site (RBS). When a non-functional terminator sequence is present the expression of RFP and GFP is similar to that observed when a terminator is absent (the NoT control). However, when a functional transcription terminator is inserted between the GFP and RFP genes the expression of RFP is attenuated. The percent attenuation, relative to GFP after normalization (using the fluorescence of the NoT control) indicates the strength of the terminator sequence.

FIG. 33 shows results of terminator functionality tests. Bars represent average (+1 s.d.) relative GFP or RFP fluorescence of S. spinosa terminator (T1-T12) or No-Terminator (NoT) cassette strains after 48 hours of growth in liquid culture. Fluorescence, of replicate cultures, was measured in 96-well assay plates on a Tecan Infinite M1000 Pro (Life Sciences) plate reader. Fluorescence was normalized to OD (OD540) and reported as relative fluorescence (as a proportion of GFP or RFP fluorescence of the NoT, control cultures). Attenuation of the GFP fluorescence relative to NoT reflects the influence of the terminator sequence on expression of the upstream gene (dasherGFP), presumably by influencing the stability of the mRNA. The attenuation of RFP fluorescence, relative to GFP, within a strain reflects the strength of the terminator—its ability to terminate transcription. Of the sequences tested, T1 performed the best, resulting in approximately an 86% reduction in expression of RFP, relative to GFP while <30% reduction in GFP expression. In contrast, T2, T4 and T8 appear to be non-functional as transcription terminators as they failed to attenuate expression of RFP. Bars represent means+/−1 SD.

FIG. 34 shows a correlation plot of relative normalized GFP vs relative normalized RFP fluorescence for each of the terminators and two strain backgrounds. The dashed line represents a 1:1 correlation. Points below the line indicate strains for which GFP>RFP (indicate attenuation of RFP fluorescence). Distance below this line (red shading) indicates relative terminator strength. Density ellipses indicate 90% confidence intervals. This plot allows visualization of relative terminator strengths.

FIG. 35 illustrates that the gusA reporter works in S. spinosa. The bars indicate mean gusA activity (+/−1 stdev), as indicated by absorbance at 405 nm, after incubation of cell free lysate from ermE*>gusA strains created in two different parent strains (A and B). The absorbance at 405 nm is proportional to yellow color resulting from the enzymatic activity of gusA acting upon 4-Nitrophenyl β-D-glucuronide substrate.

FIG. 36 illustrates endogenous fluorescence of S. spinosa. The figure represents relative fluorescence measured by fluorescence scans of a culture S. spinosa cells after washing with PBS. Curves represent fluorescence resulting from excitation at 20 nm intervals from 350-690 nm. Fluorescence is relatively strong below 500 nm but decreases with increasing excitation wavelength. In the range relevant for DasherGFP and PaprikaRFP the endogenous fluorescence is minimal. For these experiments DasherGFP was excited at 505 nm and emission was captured between 525-545 nm. This is most comparable to the curve beginning at ˜510 nm. PaprikaRFP was excited at 564 nm and fluorescence was captured between 585-610 nm. In this rang almost no endogenous fluorescence is observed.

FIG. 37 illustrates plasmid maps of pCM32, pSE101 and pSE211. (1) Plasmid maps of pCM32 (left) and the conjugation plasmid containing the pCM32 excisionase (xis), integrase (int) and attachment site (attP). The boxed part indicates the region of the plasmid that was cloned into the conjugation vector to test integration (from Chen et al., Applied Microbiology and Biotechnology. PMID 26260388 DOI: 10.1007/s00253-015-6871-z); (2) a linear map of S. erythraea plasmid pSE101. The integrase (int) and attachment site (attP) are shown at the left end of the map (from Te Poele et al., (2008) Actinomycete integrative and conjugative elements. Antonie Van Leeuwenhoek 94, 127-143); (3) a linear map of S. erythraea plasmid pSE211. The integrase (int) and attachment site (attP) are shown at the left end of the map (from Te Poele et al.).

FIG. 38 shows results of a nucleotide blast (Blastn) of the pCM32 attachment site against the S. spinosa genome. A site with greater than 99% identity (149/150 bp) is found in S. spinosa.

FIG. 39 shows results of a nucleotide blast (Blastn) of the pSE101 attachment site against the S. spinosa genome. A site with greater than 94% identity (104/111 bp) and 100% identity in the core 76 nucleotides is found in S. spinosa.

FIG. 40 shows results of a nucleotide blast (Blastn) of the pSE211 attachment site against the S. spinosa genome. A site with greater than 88% identity (122/138 bp) and 100% identity in the core 76 nucleotides is found in S. spinosa.

FIG. 41A shows Linear maps of S. erythraea replicating plasmids (AICEs) pSE101 and pSE211 (adopted from Te Poele et al., (2008) Actinomycete integrative and conjugative elements. Antonie Van Leeuwenhoek 94, 127-143), which are self-replicating plasmids to be used in S. spinosa. Arrows with diagonal lines represent genes thought to be involved in DNA replication. FIG. 41B shows schematic of an exemplary replicating plasmid containing the S. erythraea chromosomal origin of replication. To test whether the S. erythraea origin of replication can maintain replication of a plasmid in S. spinosa, the S. erythraea origin of replication will be cloned into a plasmid containing a kanamycin resistance gene, an E. coli origin of replication (pBR322) and an origin of transfer (oriT) to enable delivery of the plasmid by conjugation.

FIG. 42 shows schematic of the plasmid design, assay used for evaluation of functionality, and results of our RBS library screen. We designed and built 32 integration plasmids (31 containing and RBS and a No-RBS control). These were constructed by scarlessly cloning each RBS into a S. spinosa integration backbone between the ermE* promoter and the gene encoding levansucrase (sacB). Resulting strains were grown for 48 hours in liquid culture and serial dilutions were plated onto TSA and TSA+5% sucrose Omni Trays. If the RBS was functional, sacB was expressed leading to toxicity (absence of growth) when grown on sucrose. By comparing growth of strains containing the RBSs to a positive (strain containing the sacB RBS) and negative (No-RBS) controls, we were able to determine the relative strength of the RBS. Using this assay we identified 19 function—16 “functional” and 3 “less functional” RBSs. Results of these analysis is shown in FIG. 43A to FIG. 43E below.

FIG. 43A to FIG. 43E depict RBSs function analysis results of sucrose sensitivity assays—comparison of growth on TSA+Kan100 vs. TSA+Kan100+5% sucrose for S. spinosa RBS loop-in strains.

FIG. 44 depicts linear maps of plasmids for transposon mutagenesis in S. spinosa. Loss-of-Function (LoF) transposon, Gain-of-Function (GoF) transposon, and Gain-of-Function (GoF) Recyclable Transposon are shown.

FIG. 45 depicts an example of section of the heat map of average gene expression across the S. spinosa genome that was used to identify potential neutral integration sites.

FIG. 46 depicts an example showing that the presence of a product (e.g., Spinosyn J/L) inhibits S. spinosa growth at 1/100th the concentration in tanks.

FIG. 47 depicts selection of strains in the presence of spinosyn J/L produced isolates that grow better than the parent in the presence of spinosyn J/L.

FIG. 48A and FIG. 48B shows that selections on both spinosyn J/L (FIG. 48A) and aMM (FIG. 48B) produced strains with better performance than parent in HTP plate fermentation model.

FIG. 49A to FIG. 49C depict the process of creating scarless Saccharopolyspora spinosa strains using sacB or pheS as the counterselection mark. FIG. 49A shows introducing plasmid into S. spinosa genome using homologous recombination. FIG. 49B shows selecting for single-crossover integration events using positive selection. FIG. 49C shows using negative selection to obtain strains that have recombined to lose plasmid backbone, thus creating a scarless engineered strain.

FIG. 50 is a demonstration that sacB confers sensitivity of S. spinosa to the respective counterselection agent sucrose. Strains with or without sacB gene were tested for sucrose sensitivity at 5%. A culture dilution series were spotted in six replicates onto TSA/Kan100 and TSA or TSA/Kan100 containing 5% sucrose. It causes restrictive growth of strain expressing the gene on selective media containing 5% sucrose. “*” in the figure indicates this strain was subcultured with no selection.

FIG. 51 is a demonstration that pheS confers sensitivity of S. spinosa to the respective counterselection agent 4CP in strain A. Strain A/PheS(SS) and strain A/Phe(SE) were tested for 4CP sensitivity at 2 g/L. A culture dilution series were spotted in six replicates onto TSA/Kan100 and TSA/Kan100 containing 4CP. SE denotes pheS gene from S. erythraea, and SS denotes pheS gene from S. spinosa. After two weeks of incubation, both PheS expressing strain A-derivatives are growth inhibited on TSA/Kan100-4CP, but unaffected on TSA/Kan100. This indicates that PheS(SS) and PheS(SE) have the potential to serve as counterselection markers in S. spinosa.

FIG. 52 shows strain QC results of strains engineered in HTP using sacB as the counterselection marker. 62 engineered strain A and 14 engineered strain B were made.

FIG. 53 is a similarity matrix computed using the correlation measure done in Coynebacterium. However, similar procedures have been customized for Saccharopolyspora and are being successfully carried out by the inventors. The matrix is a representation of the functional similarity between SNP variants. The consolidation of SNPs with low functional similarity is expected to have a higher likelihood of improving strain performance, as opposed to the consolidation of SNPs with higher functional similarity.

FIG. 54A to FIG. 54B depicts the results of an epistasis mapping experiment done in Coynebacterium. However, similar procedures have been customized for Saccharopolyspora and are being successfully carried out by the inventors. Combination of SNPs and PRO swaps with low functional similarities yields improved strain performance. FIG. 54A depicts a dendrogram clustered by functional similarity of all the SNPs/PRO swaps. FIG. 54B depicts host strain performance of consolidated SNPs as measured by product yield. Greater cluster distance correlates with improved consolidation performance of the host strain.

FIG. 55 shows factors considered to improve conjugation efficiency using a design of experiment (DOE) approach.

FIG. 56A to FIG. 56B shows growth of E. coli S17+SS015 donor cells in HTP format (FIG. 56A), and results from conjugation experiment using E. coli S17+SS015 donor cells in HTP format (FIG. 56B).

FIG. 57 shows colonies identified using Qpix parameters for detection described in HTP Conjugation protocol.

FIG. 58 shows growth of S. spinosa cultures, inoculated from patches, after growth in HTP format.

FIG. 59 shows results of conjugation experiments completed through course of DOE-based optimization.

FIG. 60 shows conditions determined to be implicated in conjugation efficiency per JMP partition modeling analysis.

FIG. 61 depicts improved spinosyn J+L titer in strains engineered with SNP swap as described herein. SNP swap (SNPSWP) strains were engineered by identifying SNPs present in a late strain compared to an early (pre-mutagenesis) strain lineage and removing these from the late strain (7000153593). Selected SNPSWP strains showed improvement over parent strain (7000153593) when tested in high-throughput assay for spinosyn production. In this case, 7000153593 is both a “late strain” and the parent strain of the resulting SNPSWPs. “Late strain” is mentioned because of the principle of SNP swiping relying on early and late lineages.

FIG. 62 depicts improved spinosyn J+L titer in strains engineered with terminators as described herein. Terminator insertion strains were engineered by introducing the terminators listed in Table 9 about 25 bp in front of a number of gene targets. Select terminator insertion strains showed improvement over parent strain (7000153593) when tested in high-throughput assay for spinosyn production.

FIG. 63 depicts improved spinosyn J+L titer in strains engineered with RBS sequences as described herein. RBS swap (RBSSWP) strains were engineered by introducing the RBSs listed in Table 11 about 0 to 15 bp in front of core biosynthetic gene targets. Select RBSSWP strains showed improvement over parent strain (7000153593) when tested in high-throughput assay for spinosyn production.

FIG. 64A to FIG. 64C depict that multiple backbones were cloned to include different configurations of selection markers and genetic elements to control expression (terminators and promoters), which may alter strain engineering efficacy in different strain backgrounds. In some cases, backbones were cloned with homology arms at different sites of integration to test the effect of genomic site on backbone efficacy Promoters pD1-7, Perm2, and Perm8 and Terminator A_T are previously characterized promoters; other genetic elements listed here are cited in this work.

FIG. 65 depicts expression cassette used to evaluate the application of the terminator library for the knock down (attenuation or prevention) of gene expression.

FIG. 66A to FIG. 66B depict insertion of terminators between promoters and the coding sequence of GFP result in attenuation of GFP expression (fluorescence). Normalized GFP fluorescence of strains (means+/−95% confidence intervals) with genomic integration of the terminator knockdown GFP test cassettes are shown. FIG. 66A shows expression of strains with T1, T3, T5, T11 and T12 (SEQ ID Nos. 70, 72, 74, 79 & 80) inserted between a strong promoter (SEQ ID No. 25) and GFP. “None” (left column) indicates the no-terminator control strain. FIG. 66B shows expression of strains with T1, T3, T5 and T12 (SEQ ID Nos. 70, 72, 74 & 80) inserted between a moderately strong promoter (SEQ ID No. 33) and GFP. “None” (left column) indicates the no-terminator control strain. Standard deviations are indicated by the horizontal dashes, typically observed above and below the diamonds. Circles on the rights side of the figure indicate significant differences between groups (non-overlapping/intersecting circles indicate groups that are significantly different from each other) based on Tukey-Kramer HSD test of all pairs.

FIG. 67 depicts product titer (spinosyns J+L) of strain B-derived strains with SNPswap payloads integrated at the indicated neutral site. Strains with integration at sites 1, 2, 3, 4, 6, 9 & 10 have similar product titers and do not differ from the expected titer (average titer of strain B; higher bar on the figure). Integration at neutral site 7 appears to have a negative impact on product titer. Mean diamonds indicate the group mean and 95% confidence interval. Standard deviations are indicated by the horizontal dashes, typically observed above and below the diamonds. Circles on the rights side of the figure indicate significant differences between groups (non-overlapping/intersecting circles indicate groups that are significantly different from each other) based on Tukey-Kramer HSD test of all pairs.

FIG. 68 depicts comparison of GFP expression when integrated at the indicated neutral sites. Data represents normalized fluorescence of WT and B-derived strain with a GFP expression cassette—a strong promoter (SEQ ID No. 25) driving expression of GFP (SEQ ID No. 81)—integrated at the indicted neutral sites. P1-control indicates fluorescence of this cassette integrated at previously reported neutral site. Expression is similar at most sites. Only NS7 was significantly different from other neutral sites we evaluated (NS2, NS3, NS4, NS6, and NS10). Standard deviations are indicated by the horizontal dashes, typically observed above and below the diamonds. Circles on the rights side of the figure indicate significant differences between groups (non-overlapping/intersecting circles indicate groups that are significantly different from each other) based on Tukey-Kramer HSD test of all pairs

FIG. 69 depicts that strains engineered by anti-metabolite selection were tested for performance of spinosyn production. All strains showed reduction in performance of spinosyn production with respect to parent. This approach needs optimization to identify strains.

### DETAILED DESCRIPTION

### Definitions

While the following terms are believed to be well understood by one of ordinary skill in the art, the following definitions are set forth to facilitate explanation of the presently disclosed subject matter.

The term “a” or “an” refers to one or more of that entity, i.e. can refer to a plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein the terms “cellular organism” “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure refers to the “microorganisms” or “cellular organisms” or “microbes” of lists/tables and figures present in the disclosure. This characterization can refer to not only the identified taxonomic genera of the tables and figures, but also the identified taxonomic species, as well as the various novel and newly identified or designed strains of any organism in said tables or figures. The same characterization holds true for the recitation of these terms in other parts of the Specification, such as in the Examples.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

The terms “genetically modified host cell,” “recombinant host cell,” and “recombinant strain” are used interchangeably herein and refer to host cells that have been genetically modified by the cloning and transformation methods of the present disclosure. Thus, the terms include a host cell (e.g., bacteria, yeast cell, fungal cell, CHO, human cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring organism from which it was derived. It is understood that in some embodiments, the terms refer not only to the particular recombinant host cell in question, but also to the progeny or potential progeny of such a host cell

The term “wild-type microorganism” or “wild-type host cell” describes a cell that occurs in nature, i.e. a cell that has not been genetically modified.

The term “genetically engineered” may refer to any manipulation of a host cell's genome (e.g. by insertion, deletion, mutation, or replacement of nucleic acids).

The term “control” or “control host cell” refers to an appropriate comparator host cell for determining the effect of a genetic modification or experimental treatment. In some embodiments, the control host cell is a wild type cell. In other embodiments, a control host cell is genetically identical to the genetically modified host cell, save for the genetic modification(s) differentiating the treatment host cell. In some embodiments, the present disclosure teaches the use of parent strains as control host cells (e.g., the S₁ strain that was used as the basis for the strain improvement program). In other embodiments, a host cell may be a genetically identical cell that lacks a specific promoter or SNP being tested in the treatment host cell.

The term “production strain” or “production microbe” as used herein refers to a host cell that comprises one or more genetic differences from a wild-type or control host cell organism that improve the performance of the production strain (e.g., that make the strain a better candidate for commercial production of one or more compounds). In some embodiments the production strain will be a strain currently used in commercial production. In some embodiments, the production strain will be an organism that has undergone one or more rounds of mutations/genetic engineering to improve the properties of the strain.

As used herein, the term “allele(s)” means any of one or more alternative forms of a gene, all of which alleles relate to at least one trait or characteristic. In a diploid cell, the two alleles of a given gene occupy corresponding loci on a pair of homologous chromosomes.

As used herein, the term “locus” (loci plural) means a specific place or places or a site on a chromosome where for example a gene or genetic marker is found.

As used herein, the term “genetically linked” refers to two or more traits that are co-inherited at a high rate during breeding such that they are difficult to separate through crossing.

A “recombination” or “recombination event” as used herein refers to a chromosomal crossing over or independent assortment.

As used herein, the term “phenotype” refers to the observable characteristics of an individual cell, cell culture, organism, or group of organisms which results from the interaction between that individual's genetic makeup (i.e., genotype) and the environment.

As used herein, the term “chimeric” or “recombinant” when describing a nucleic acid sequence or a protein sequence refers to a nucleic acid, or a protein sequence, that links at least two heterologous polynucleotides, or two heterologous polypeptides, into a single macromolecule, or that re-arranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “endogenous” or “endogenous gene,” refers to the naturally occurring gene, in the location in which it is naturally found within the host cell genome. In the context of the present disclosure, operably linking a heterologous promoter to an endogenous gene means genetically inserting a heterologous promoter sequence in front of an existing gene, in the location where that gene is naturally present. An endogenous gene as described herein can include alleles of naturally occurring genes that have been mutated according to any of the methods of the present disclosure.

As used herein, the term “exogenous” is used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458.

For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3^{rd} ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

As used herein, “promoter” refers to a DNA sequence capable of controlling the expression of a coding sequence or functional RNA. In some embodiments, the promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

“Operably linked” means in this context the sequential arrangement of the promoter polynucleotide according to the disclosure with a further oligo- or polynucleotide, resulting in transcription of said further polynucleotide.

The term “product of interest” or “biomolecule” as used herein refers to any product produced by microbes from feedstock. In some cases, the product of interest may be a small molecule, enzyme, peptide, amino acid, organic acid, synthetic compound, fuel, alcohol, etc. For example, the product of interest or biomolecule may be any primary or secondary extracellular metabolite. The primary metabolite may be, inter alia, ethanol, citric acid, lactic acid, glutamic acid, glutamate, lysine, spinosyns, spinetoram, threonine, tryptophan and other amino acids, vitamins, polysaccharides, etc. The secondary metabolite may be, inter alia, an antibiotic compound like penicillin, or an immunosuppressant like cyclosporin A, a plant hormone like gibberellin, a statin drug like lovastatin, a fungicide like griseofulvin, etc. The product of interest or biomolecule may also be any intracellular component produced by a microbe, such as: a microbial enzyme, including: catalase, amylase, protease, pectinase, glucose isomerase, cellulase, hemicellulase, lipase, lactase, streptokinase, and many others. The intracellular component may also include recombinant proteins, such as: insulin, hepatitis B vaccine, interferon, granulocyte colony-stimulating factor, streptokinase and others.

The term “carbon source” generally refers to a substance suitable to be used as a source of carbon for cell growth. Carbon sources include, but are not limited to, biomass hydrolysates, starch, sucrose, cellulose, hemicellulose, xylose, and lignin, as well as monomeric components of these substrates. Carbon sources can comprise various organic compounds in various forms, including, but not limited to polymers, carbohydrates, acids, alcohols, aldehydes, ketones, amino acids, peptides, etc. These include, for example, various monosaccharides such as glucose, dextrose (D-glucose), maltose, oligosaccharides, polysaccharides, saturated or unsaturated fatty acids, succinate, lactate, acetate, ethanol, etc., or mixtures thereof. Photosynthetic organisms can additionally produce a carbon source as a product of photosynthesis. In some embodiments, carbon sources may be selected from biomass hydrolysates and glucose.

The term “feedstock” is defined as a raw material or mixture of raw materials supplied to a microorganism or fermentation process from which other products can be made. For example, a carbon source, such as biomass or the carbon compounds derived from biomass are a feedstock for a microorganism that produces a product of interest (e.g. small molecule, peptide, synthetic compound, fuel, alcohol, etc.) in a fermentation process. However, a feedstock may contain nutrients other than a carbon source.

The term “volumetric productivity” or “production rate” is defined as the amount of product formed per volume of medium per unit of time. Volumetric productivity can be reported in gram per liter per hour (g/L/h).

The term “specific productivity” is defined as the rate of formation of the product. Specific productivity is herein further defined as the specific productivity in gram product per gram of cell dry weight (CDW) per hour (g/g CDW/h). Using the relation of CDW to OD₆₀₀ for the given microorganism specific productivity can also be expressed as gram product per liter culture medium per optical density of the culture broth at 600 nm (OD) per hour (g/L/h/OD).

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product.

The term “titre” or “titer” is defined as the strength of a solution or the concentration of a substance in solution. For example, the titre of a product of interest (e.g. small molecule, peptide, synthetic compound, fuel, alcohol, etc.) in a fermentation broth is described as g of product of interest in solution per liter of fermentation broth (g/L).

The term “total titer” is defined as the sum of all product of interest produced in a process, including but not limited to the product of interest in solution, the product of interest in gas phase if applicable, and any product of interest removed from the process and recovered relative to the initial volume in the process or the operating volume in the process

As used herein, the term “HTP genetic design library” or “library” refers to collections of genetic perturbations according to the present disclosure. In some embodiments, the libraries of the present invention may manifest as i) a collection of sequence information in a database or other computer file, ii) a collection of genetic constructs encoding for the aforementioned series of genetic elements, or iii) host cell strains comprising said genetic elements. In some embodiments, the libraries of the present disclosure may refer to collections of individual elements (e.g., collections of promoters for PRO swap libraries, or collections of terminators for STOP swap libraries). In other embodiments, the libraries of the present disclosure may also refer to combinations of genetic elements, such as combinations of promoter::genes, gene:terminator, or even promoter:gene:terminators. In some embodiments, the libraries of the present disclosure further comprise meta data associated with the effects of applying each member of the library in host organisms. For example, a library as used herein can include a collection of promoter::gene sequence combinations, together with the resulting effect of those combinations on one or more phenotypes in a particular species, thus improving the future predictive value of using said combination in future promoter swaps.

As used herein, the term “SNP” refers to Small Nuclear Polymorphism(s). In some embodiments, SNPs of the present disclosure should be construed broadly, and include single nucleotide polymorphisms, sequence insertions, deletions, inversions, and other sequence replacements. As used herein, the term “non-synonymous” or non-synonymous SNPs” refers to mutations that lead to coding changes in host cell proteins. In some embodiments SNPs of the present disclosure comprise additional copies of one or more genes (e.g., copies of one or more polynucleotides encoding for biosynthetic enzyme genes).

A “high-throughput (HTP)” method of genomic engineering may involve the utilization of at least one piece of automated equipment (e.g. a liquid handler or plate handler machine) to carry out at least one step of said method.

A “scarless genomic editing” or “scarless gene replacement” refers to a method of editing a specific genomic sequence of a given species, without introducing any marker sequence or any plasmid backbone sequence into the genome of the species after the desired genome editing is accomplished. The genomic editing can be a substitution, a deletion, and/or addition of one or more nucleic acids of the genome.

### Traditional Methods of Strain Improvement

Traditional approaches to strain improvement can be broadly categorized into two types of approaches: directed strain engineering, and random mutagenesis.

Directed engineering methods of strain improvement involve the planned perturbation of a handful of genetic elements of a specific organism. These approaches are typically focused on modulating specific biosynthetic or developmental programs, and rely on prior knowledge of the genetic and metabolic factors affecting said pathways. In its simplest embodiments, directed engineering involves the transfer of a characterized trait (e.g., gene, promoter, or other genetic element capable of producing a measurable phenotype) from one organism to another organism of the same, or different species.

Random approaches to strain engineering involve the random mutagenesis of parent strains, coupled with extensive screening designed to identify performance improvements. Approaches to generating these random mutations include exposure to ultraviolet radiation, or mutagenic chemicals such as Ethyl methanesulfonate. Though random and largely unpredictable, this traditional approach to strain improvement had several advantages compared to more directed genetic manipulations. First, many industrial organisms were (and remain) poorly characterized in terms of their genetic and metabolic repertoires, rendering alternative directed improvement approaches difficult, if not impossible.

Second, even in relatively well characterized systems, genotypic changes that result in industrial performance improvements are difficult to predict, and sometimes only manifest themselves as epistatic phenotypes requiring cumulative mutations in many genes of known and unknown function.

Additionally, for many years, the genetic tools required for making directed genomic mutations in a given industrial organism were unavailable, or very slow and/or difficult to use.

The extended application of the traditional strain improvement programs, however, yield progressively reduced gains in a given strain lineage, and ultimately lead to exhausted possibilities for further strain efficiencies. Beneficial random mutations are relatively rare events, and require large screening pools and high mutation rates. This inevitably results in the inadvertent accumulation of many neutral and/or detrimental (or partly detrimental) mutations in “improved” strains, which ultimately create a drag on future efficiency gains.

Another limitation of traditional cumulative improvement approaches is that little to no information is known about any particular mutation's effect on any strain metric. This fundamentally limits a researcher's ability to combine and consolidate beneficial mutations, or to remove neutral or detrimental mutagenic “baggage.”

Other approaches and technologies exist to randomly recombine mutations between strains within a mutagenic lineage. For example, some formats and examples for iterative sequence recombination, sometimes referred to as DNA shuffling, evolution, or molecular breeding, have been described in U.S. patent application Ser. No. 08/198,431, filed Feb. 17, 1994, Serial No. PCT/US95/02126, filed, Feb. 17, 1995, Ser. No. 08/425,684, filed Apr. 18, 1995, Ser. No. 08/537,874, filed Oct. 30, 1995, Ser. No. 08/564,955, filed Nov. 30, 1995, Ser. No. 08/621,859, filed. Mar. 25, 1996, Ser. No. 08/621,430, filed Mar. 25, 1996, Serial No. PCT/US96/05480, filed Apr. 18, 1996, Ser. No. 08/650,400, filed May 20, 1996, Ser. No. 08/675,502, filed Jul. 3, 1996, Ser. No. 08/721,824, filed Sep. 27, 1996, and Ser. No. 08/722,660 filed Sep. 27, 1996; Stemmer, Science 270:1510 (1995); Stemmer et al., Gene 164:49-53 (1995); Stemmer, Bio/Technology 13:549-553 (1995); Stemmer, Proc. Natl. Acad. Sci. U.S.A. 91:10747-10751 (1994); Stemmer, Nature 370:389-391 (1994); Crameri et al., Nature Medicine 2(1):1-3 (1996); Crameri et al., Nature Biotechnology 14:315-319 (1996), each of which is incorporated herein by reference in its entirety for all purposes.

These include techniques such as protoplast fusion and whole genome shuffling that facilitate genomic recombination across mutated strains. For some industrial microorganisms such as yeast and filamentous fungi, natural mating cycles can also be exploited for pairwise genomic recombination. In this way, detrimental mutations can be removed by ‘back-crossing’ mutants with parental strains and beneficial mutations consolidated. Moreover, beneficial mutations from two different strain lineages can potentially be combined, which creates additional improvement possibilities over what might be available from mutating a single strain lineage on its own.

To provide additional improvements beyond traditional strain improvement programs, the present disclosure sets forth a unique HTP genomic engineering platform that is computationally driven and integrates molecular biology, automation, data analytics, and machine learning protocols. This integrative platform utilizes a suite of HTP molecular tool sets that are used to construct HTP genetic design libraries. These genetic design libraries will be elaborated upon below.

The taught HTP platform and its unique microbial genetic design libraries fundamentally shift the paradigm of microbial strain development and evolution. For example, traditional mutagenesis-based methods of developing an industrial microbial strain will eventually lead to microbes burdened with a heavy mutagenic load that has been accumulated over years of random mutagenesis.

The ability to solve this issue (i.e. remove the genetic baggage accumulated by these microbes) has eluded microbial researchers for decades. However, utilizing the HTP platform disclosed herein, these industrial strains can be “rehabilitated,” and the genetic mutations that are deleterious can be identified and removed. Congruently, the genetic mutations that are identified as beneficial can be kept, and in some cases improved upon. The resulting microbial strains demonstrate superior phenotypic traits (e.g., improved production of a compound of interest), as compared to their parental strains.

Furthermore, the HTP platform taught herein is able to identify, characterize, and quantify the effect that individual mutations have on microbial strain performance. This information, i.e. what effect does a given genetic change x have on host cell phenotype y (e.g., production of a compound or product of interest), is able to be generated and then stored in the microbial HTP genetic design libraries discussed below. That is, sequence information for each genetic permutation, and its effect on the host cell phenotype are stored in one or more databases, and are available for subsequent analysis (e.g., epistasis mapping, as discussed below). The present disclosure also teaches methods of physically saving/storing valuable genetic permutations in the form of genetic insertion constructs, or in the form of one or more host cell organisms containing said genetic permutation (e.g., see libraries discussed below.)

When one couples these HTP genetic design libraries into an iterative process that is integrated with a sophisticated data analytics and machine learning process a dramatically different methodology for improving host cells emerges. The taught platform is therefore fundamentally different from the previously discussed traditional methods of developing host cell strains. The taught HTP platform does not suffer from many of the drawbacks associated with the previous methods. These and other advantages will become apparent with reference to the HTP molecular tool sets and the derived genetic design libraries discussed below.

### Genetic Design & Microbial Engineering: A Systematic Combinatorial Approach to Strain Improvement Utilizing a Suite of HTP Molecular Tools and HTP Genetic Design Libraries

As aforementioned, the present disclosure provides a novel HTP platform and genetic design strategy for engineering microbial organisms through iterative systematic introduction and removal of genetic changes across strains. The platform is supported by a suite of molecular tools, which enable the creation of HTP genetic design libraries and allow for the efficient implementation of genetic alterations into a given host strain.

The HTP genetic design libraries of the disclosure serve as sources of possible genetic alterations that may be introduced into a particular microbial strain background. In this way, the HTP genetic design libraries are repositories of genetic diversity, or collections of genetic perturbations, which can be applied to the initial or further engineering of a given microbial strain. Techniques for programming genetic designs for implementation to host strains are described in pending U.S. patent application Ser. No. 15/140,296, entitled “Microbial Strain Design System and Methods for Improved Large Scale Production of Engineered Nucleotide Sequences,” incorporated by reference in its entirety herein.

The HTP molecular tool sets utilized in this platform may include, inter alia: (1) Promoter swaps (PRO Swap), (2) SNP swaps, (3) Start/Stop codon exchanges, (4) STOP swaps, (5) Sequence optimization, (6) transposon mutagenesis diversity libraries, (7) ribosomal binding site (RBS) diversity libraries, and (8) anti-metabolite selection/fermentation product resistance libraries. The HTP methods of the present disclosure also teach methods for directing the consolidation/combinatorial use of HTP tool sets, including (9) Epistasis mapping protocols. As aforementioned, this suite of molecular tools, either in isolation or combination, enables the creation of HTP genetic design host cell libraries.

As will be demonstrated, utilization of the aforementioned HTP genetic design libraries in the context of the taught HTP microbial engineering platform enables the identification and consolidation of beneficial “causative” mutations or gene sections and also the identification and removal of passive or detrimental mutations or gene sections. This new approach allows rapid improvements in strain performance that could not be achieved by traditional random mutagenesis or directed genetic engineering. The removal of genetic burden or consolidation of beneficial changes into a strain with no genetic burden also provides a new, robust starting point for additional random mutagenesis that may enable further improvements.

In some embodiments, the present disclosure teaches that as orthogonal beneficial changes are identified across various, discrete branches of a mutagenic strain lineage, they can also be rapidly consolidated into better performing strains. These mutations can also be consolidated into strains that are not part of mutagenic lineages, such as strains with improvements gained by directed genetic engineering.

In some embodiments, the present disclosure differs from known strain improvement approaches in that it analyzes the genome-wide combinatorial effect of mutations across multiple disparate genomic regions, including expressed and non-expressed genetic elements, and uses gathered information (e.g., experimental results) to predict mutation combinations expected to produce strain enhancements.

In some embodiments, the present disclosure teaches: i) industrial microorganisms, and other host cells amenable to improvement via the disclosed inventions, ii) generating diversity pools for downstream analysis, iii) methods and hardware for high-throughput screening and sequencing of large variant pools, iv) methods and hardware for machine learning computational analysis and prediction of synergistic effects of genome-wide mutations, and v) methods for high-throughput strain engineering.

The following molecular tools and libraries are discussed in terms of illustrative microbial examples. Persons having skill in the art will recognize that the HTP molecular tools of the present disclosure are compatible with any host cell, including eukaryotic cellular, and higher life forms.

Each of the identified HTP molecular tool sets—which enable the creation of the various HTP genetic design libraries utilized in the microbial engineering platform—will now be discussed.

1. Promoter Swaps: A Molecular Tool for the Derivation of Promoter Swap Microbial Strain Libraries

In some embodiments, the present disclosure teaches methods of selecting promoters with optimal expression properties to produce beneficial effects on overall-host strain phenotype (e.g., yield or productivity).

For example, in some embodiments, the present disclosure teaches methods of identifying one or more promoters and/or generating variants of one or more promoters within a host cell, which exhibit a range of expression strengths (e.g. promoter ladders discussed infra), or superior regulatory properties (e.g., tighter regulatory control for selected genes). A particular combination of these identified and/or generated promoters can be grouped together as a promoter ladder, which is explained in more detail below.

The promoter ladder in question is then associated with a given gene of interest. Thus, if one has promoters P₁-P₈ (representing eight promoters that have been identified and/or generated to exhibit a range of expression strengths) and associates the promoter ladder with a single gene of interest in a microbe (i.e. genetically engineer a microbe with a given promoter operably linked to a given target gene), then the effect of each combination of the eight promoters can be ascertained by characterizing each of the engineered strains resulting from each combinatorial effort, given that the engineered microbes have an otherwise identical genetic background except the particular promoter(s) associated with the target gene.

The resultant microbes that are engineered via this process form HTP genetic design libraries.

The HTP genetic design library can refer to the actual physical microbial strain collection that is formed via this process, with each member strain being representative of a given promoter operably linked to a particular target gene, in an otherwise identical genetic background, said library being termed a “promoter swap microbial strain library.”

Furthermore, the HTP genetic design library can refer to the collection of genetic perturbations—in this case a given promoter x operably linked to a given gene y—said collection being termed a “promoter swap library.”

Further, one can utilize the same promoter ladder comprising promoters in Table 1 to engineer microbes, wherein each of the promoters is operably linked to different gene targets. The result of this procedure would be microbes that are otherwise assumed genetically identical, except for the particular promoters operably linked to a target gene of interest. These microbes could be appropriately screened and characterized and give rise to another HTP genetic design library. The characterization of the microbial strains in the HTP genetic design library produces information and data that can be stored in any data storage construct, including a relational database, an object-oriented database or a highly distributed NoSQL database. This data/information could be, for example, a given promoter's effect when operably linked to a given gene target. This data/information can also be the broader set of combinatorial effects that result from operably linking two or more of promoters of the present disclosure to a given gene target.

The aforementioned examples of promoters and target genes is merely illustrative, as the concept can be applied with any given number of promoters that have been grouped together based upon exhibition of a range of expression strengths and any given number of target genes. Persons having skill in the art will also recognize the ability to operably link two or more promoters in front of any gene target. Thus, in some embodiments, the present disclosure teaches promoter swap libraries in which 1, 2, 3 or more promoters from a promoter ladder are operably linked to one or more genes.

In summary, utilizing various promoters to drive expression of various genes in an organism is a powerful tool to optimize a trait of interest. The molecular tool of promoter swapping, developed by the inventors, uses a ladder of promoter sequences that have been demonstrated to vary expression of at least one locus under at least one condition. This ladder is then systematically applied to a group of genes in the organism using high-throughput genome engineering. This group of genes is determined to have a high likelihood of impacting the trait of interest based on any one of a number of methods. These could include selection based on known function, or impact on the trait of interest, or algorithmic selection based on previously determined beneficial genetic diversity. In some embodiments, the selection of genes can include all the genes in a given host. In other embodiments, the selection of genes can be a subset of all genes in a given host, chosen randomly.

The resultant HTP genetic design microbial strain library of organisms containing a promoter sequence linked to a gene is then assessed for performance in a high-throughput screening model, and promoter-gene linkages which lead to increased performance are determined and the information stored in a database. The collection of genetic perturbations (i.e. given promoter x operably linked to a given gene y) form a “promoter swap library,” which can be utilized as a source of potential genetic alterations to be utilized in microbial engineering processing. Over time, as a greater set of genetic perturbations is implemented against a greater diversity of host cell backgrounds, each library becomes more powerful as a corpus of experimentally confirmed data that can be used to more precisely and predictably design targeted changes against any background of interest.

Transcription levels of genes in an organism are a key point of control for affecting organism behavior. Transcription is tightly coupled to translation (protein expression), and which proteins are expressed in what quantities determines organism behavior. Cells express thousands of different types of proteins, and these proteins interact in numerous complex ways to create function. By varying the expression levels of a set of proteins systematically, function can be altered in ways that, because of complexity, are difficult to predict. Some alterations may increase performance, and so, coupled to a mechanism for assessing performance, this technique allows for the generation of organisms with improved function.

In the context of a small molecule synthesis pathway, enzymes interact through their small molecule substrates and products in a linear or branched chain, starting with a substrate and ending with a small molecule of interest. Because these interactions are sequentially linked, this system exhibits distributed control, and increasing the expression of one enzyme can only increase pathway flux until another enzyme becomes rate limiting.

Metabolic Control Analysis (MCA) is a method for determining, from experimental data and first principles, which enzyme or enzymes are rate limiting. MCA is limited however, because it requires extensive experimentation after each expression level change to determine the new rate limiting enzyme. Promoter swapping is advantageous in this context, because through the application of a promoter ladder to each enzyme in a pathway, the limiting enzyme is found, and the same thing can be done in subsequent rounds to find new enzymes that become rate limiting. Further, because the read-out on function is better production of the small molecule of interest, the experiment to determine which enzyme is limiting is the same as the engineering to increase production, thus shortening development time. In some embodiments the present disclosure teaches the application of PRO swap to genes encoding individual subunits of multi-unit enzymes. In yet other embodiments, the present disclosure teaches methods of applying PRO swap techniques to genes responsible for regulating individual enzymes, or whole biosynthetic pathways.

In some embodiments, the promoter swap tool of the present disclosure can is used to identify optimum expression of a selected gene target. In some embodiments, the goal of the promoter swap may be to increase expression of a target gene to reduce bottlenecks in a metabolic or genetic pathway. In other embodiments, the goal o the promoter swap may be to reduce the expression of the target gene to avoid unnecessary energy expenditures in the host cell, when expression of said target gene is not required.

In the context of other cellular systems like transcription, transport, or signaling, various rational methods can be used to try and find out, a priori, which proteins are targets for expression change and what that change should be. These rational methods reduce the number of perturbations that must be tested to find one that improves performance, but they do so at significant cost. Gene deletion studies identify proteins whose presence is critical for a particular function, and important genes can then be over-expressed. Due to the complexity of protein interactions, this is often ineffective at increasing performance. Different types of models have been developed that attempt to describe, from first principles, transcription or signaling behavior as a function of protein levels in the cell. These models often suggest targets where expression changes might lead to different or improved function. The assumptions that underlie these models are simplistic and the parameters difficult to measure, so the predictions they make are often incorrect, especially for non-model organisms. With both gene deletion and modeling, the experiments required to determine how to affect a certain gene are different than the subsequent work to make the change that improves performance. Promoter swapping sidesteps these challenges, because the constructed strain that highlights the importance of a particular perturbation is also, already, the improved strain.

Thus, in particular embodiments, promoter swapping is a multi-step process comprising:

1. Selecting a set of “x” promoters to act as a “ladder.” Ideally these promoters have been shown to lead to highly variable expression across multiple genomic loci, but the only requirement is that they perturb gene expression in some way.

2. Selecting a set of “n” genes to target. This set can be every open reading frame (ORF) in a genome, or a subset of ORFs. The subset can be chosen using annotations on ORFs related to function, by relation to previously demonstrated beneficial perturbations (previous promoter swaps or previous SNP swaps), by algorithmic selection based on epistatic interactions between previously generated perturbations, other selection criteria based on hypotheses regarding beneficial ORF to target, or through random selection. In other embodiments, the “n” targeted genes can comprise non-protein coding genes, including non-coding RNAs.

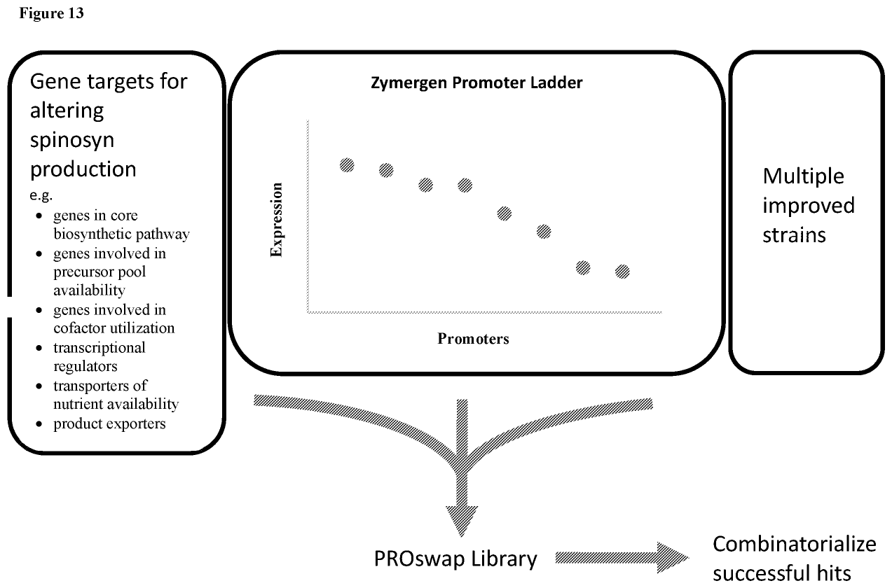

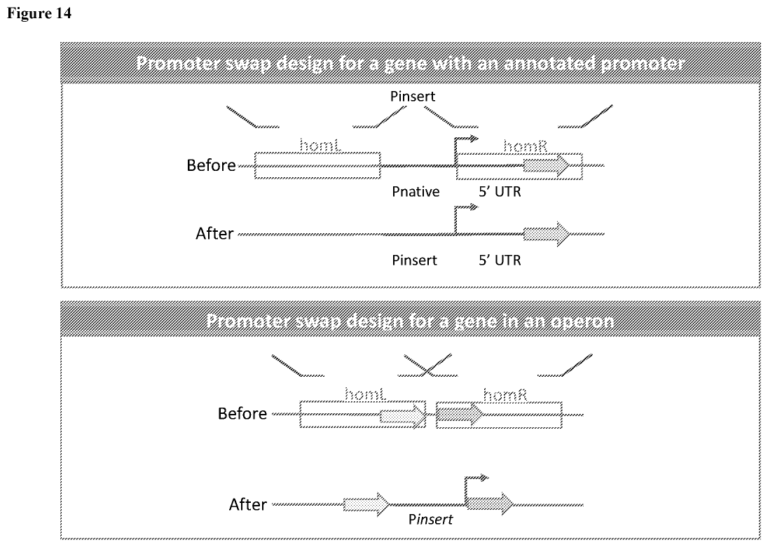

3. High-throughput strain engineering to rapidly-and in some embodiments, in parallel-carry out the following genetic modifications: When a native promoter exists in front of target gene n and its sequence is known, replace the native promoter with each of the x promoters in the ladder. When the native promoter does not exist, or its sequence is unknown, insert each of the x promoters in the ladder in front of gene n (see e.g., FIGS. 13 and 14). Thus, in some embodiments, SNP Swap libraries may be promoter insertion libraries, in which genetic elements without promoters, or with weak promoters are tested with newly added promoters. Such genes for promoter SWP library modification include, but are not limited to: (1) genes in core biosynthetic pathway of a compound of interest, such as a spinosyn; (2) genes involved in precursor pool availability of a compound of interest, such as a gene directly involved in precursor synthesis or regulation of pool availability; (3) genes involved in cofactor utilization; (4) genes encoding with transcriptional regulators; (5) genes encoding transporters of nutrient availability; and (6) product exporters, etc. In this way a “library” (also referred to as a HTP genetic design library) of strains is constructed, wherein each member of the library is an instance of x promoter operably linked to n target, in an otherwise identical genetic context. As previously described combinations of promoters can be inserted, extending the range of combinatorial possibilities upon which the library is constructed.

4. High-throughput screening of the library of strains in a context where their performance against one or more metrics is indicative of the performance that is being optimized.

This foundational process can be extended to provide further improvements in strain performance by, inter alia: (1) Consolidating multiple beneficial perturbations into a single strain background, either one at a time in an interactive process, or as multiple changes in a single step. Multiple perturbations can be either a specific set of defined changes or a partly randomized, combinatorial library of changes. For example, if the set of targets is every gene in a pathway, then sequential regeneration of the library of perturbations into an improved member or members of the previous library of strains can optimize the expression level of each gene in a pathway regardless of which genes are rate limiting at any given iteration; (2) Feeding the performance data resulting from the individual and combinatorial generation of the library into an algorithm that uses that data to predict an optimum set of perturbations based on the interaction of each perturbation; and (3) Implementing a combination of the above two approaches (see FIG. 13).

The molecular tool, or technique, discussed above is characterized as promoter swapping, but is not limited to promoters and can include other sequence changes that systematically vary the expression level of a set of targets. Other methods for varying the expression level of a set of genes could include: a) a ladder of ribosome binding sites (or Kozak sequences in eukaryotes); b) replacing the start codon of each target with each of the other start codons (i.e start/stop codon exchanges discussed infra); c) attachment of various mRNA stabilizing or destabilizing sequences to the 5′ or 3′ end, or at any other location, of a transcript, d) attachment of various protein stabilizing or destabilizing sequences at any location in the protein.

The approach is exemplified in the present disclosure with industrial microorganisms, but is applicable to any organism where desired traits can be identified in a population of genetic mutants. For example, this could be used for improving the performance of CHO cells, yeast, insect cells, algae, as well as multi-cellular organisms, such as plants.

2. SNP Swaps: A Molecular Tool for the Derivation of SNP Swap Microbial Strain Libraries

In certain embodiments, SNP swapping is not a random mutagenic approach to improving a microbial strain, but rather involves the systematic introduction or removal of individual Small Nuclear Polymorphism nucleotide mutations (i.e. SNPs) (hence the name “SNP swapping”) across strains.

The resultant microbes that are engineered via this process form HTP genetic design libraries.

The HTP genetic design library can refer to the actual physical microbial strain collection that is formed via this process, with each member strain being representative of the presence or absence of a given SNP, in an otherwise identical genetic background, said library being termed a “SNP swap microbial strain library.”

Furthermore, the HTP genetic design library can refer to the collection of genetic perturbations—in this case a given SNP being present or a given SNP being absent—said collection being termed a “SNP swap library.”

In some embodiments, SNP swapping involves the reconstruction of host organisms with optimal combinations of target SNP “building blocks” with identified beneficial performance effects. Thus, in some embodiments, SNP swapping involves consolidating multiple beneficial mutations into a single strain background, either one at a time in an iterative process, or as multiple changes in a single step. Multiple changes can be either a specific set of defined changes or a partly randomized, combinatorial library of mutations.

In other embodiments, SNP swapping also involves removing multiple mutations identified as detrimental from a strain, either one at a time in an iterative process, or as multiple changes in a single step. Multiple changes can be either a specific set of defined changes or a partly randomized, combinatorial library of mutations. In some embodiments, the SNP swapping methods of the present disclosure include both the addition of beneficial SNPs, and removing detrimental and/or neutral mutations.

SNP swapping is a powerful tool to identify and exploit both beneficial and detrimental mutations in a lineage of strains subjected to mutagenesis and selection for an improved trait of interest. SNP swapping utilizes high-throughput genome engineering techniques to systematically determine the influence of individual mutations in a mutagenic lineage. Genome sequences are determined for strains across one or more generations of a mutagenic lineage with known performance improvements. High-throughput genome engineering is then used systematically to recapitulate mutations from improved strains in earlier lineage strains, and/or revert mutations in later strains to earlier strain sequences. The performance of these strains is then evaluated and the contribution of each individual mutation on the improved phenotype of interest can be determined. As aforementioned, the microbial strains that result from this process are analyzed/characterized and form the basis for the SNP swap genetic design libraries that can inform microbial strain improvement across host strains.

Removal of detrimental mutations can provide immediate performance improvements, and consolidation of beneficial mutations in a strain background not subject to mutagenic burden can rapidly and greatly improve strain performance. The various microbial strains produced via the SNP swapping process form the HTP genetic design SNP swapping libraries, which are microbial strains comprising the various added/deleted/or consolidated SNPs, but with otherwise identical genetic backgrounds.

As discussed previously, random mutagenesis and subsequent screening for performance improvements is a commonly used technique for industrial strain improvement, and many strains currently used for large scale manufacturing have been developed using this process iteratively over a period of many years, sometimes decades. Random approaches to generating genomic mutations such as exposure to UV radiation or chemical mutagens such as ethyl methanesulfonate were a preferred method for industrial strain improvements because: 1) industrial organisms may be poorly characterized genetically or metabolically, rendering target selection for directed improvement approaches difficult or impossible; 2) even in relatively well characterized systems, changes that result in industrial performance improvements are difficult to predict and may require perturbation of genes that have no known function, and 3) genetic tools for making directed genomic mutations in a given industrial organism may not be available or very slow and/or difficult to use.

However, despite the aforementioned benefits of this process, there are also a number of known disadvantages. Beneficial mutations are relatively rare events, and in order to find these mutations with a fixed screening capacity, mutations rates must be sufficiently high. This often results in unwanted neutral and partly detrimental mutations being incorporated into strains along with beneficial changes. Over time this ‘mutagenic burden’ builds up, resulting in strains with deficiencies in overall robustness and key traits such as growth rates. Eventually ‘mutagenic burden’ renders further improvements in performance through random mutagenesis increasingly difficult or impossible to obtain. Without suitable tools, it is impossible to consolidate beneficial mutations found in discrete and parallel branches of strain lineages.

SNP swapping is an approach to overcome these limitations by systematically recapitulating or reverting some or all mutations observed when comparing strains within a mutagenic lineage. In this way, both beneficial (‘causative’) mutations can be identified and consolidated, and/or detrimental mutations can be identified and removed. This allows rapid improvements in strain performance that could not be achieved by further random mutagenesis or targeted genetic engineering.

Removal of genetic burden or consolidation of beneficial changes into a strain with no genetic burden also provides a new, robust starting point for additional random mutagenesis that may enable further improvements.

In addition, as orthogonal beneficial changes are identified across various, discrete branches of a mutagenic strain lineage, they can be rapidly consolidated into better performing strains. These mutations can also be consolidated into strains that are not part of mutagenic lineages, such as strains with improvements gained by directed genetic engineering.

Other approaches and technologies exist to randomly recombine mutations between strains within a mutagenic lineage. These include techniques such as protoplast fusion and whole genome shuffling that facilitate genomic recombination across mutated strains. For some industrial microorganisms such as yeast and filamentous fungi, natural mating cycles can also be exploited for pairwise genomic recombination. In this way, detrimental mutations can be removed by ‘back-crossing’ mutants with parental strains and beneficial mutations consolidated.

The traditional approaches can be used with SNP swapping methods disclosed herein to combine random mutation discovery with the systematic introduction or removal of individual mutations across strains.

In some embodiments, the present disclosure teaches methods for identifying the SNP sequence diversity present among the organisms of a diversity pool. A diversity pool can be a given number n of microbes utilized for analysis, with said microbes' genomes representing the “diversity pool.”

In particular aspects, a diversity pool may be an original parent strain (S₁) with a “baseline” or “reference” genetic sequence at a particular time point (S₁Gen₁) and then any number of subsequent offspring strains (S₂₋ₙ) that were derived/developed from said S₁ strain and that have a different genome (S₂₋ₙGen₂₋ₙ), in relation to the baseline genome of S₁.

For example, in some embodiments, the present disclosure teaches sequencing the microbial genomes in a diversity pool to identify the SNPs present in each strain. In one embodiment, the strains of the diversity pool are historical microbial production strains. Thus, a diversity pool of the present disclosure can include for example, an industrial reference strain, and one or more mutated industrial strains produced via traditional strain improvement programs.

In some embodiments, the SNPs within a diversity pool are determined with reference to a “reference strain.” In some embodiments, the reference strain is a wild-type strain. In other embodiments, the reference strain is an original industrial strain prior to being subjected to any mutagenesis. The reference strain can be defined by the practitioner and does not have to be an original wild-type strain or original industrial strain. The base strain is merely representative of what will be considered the “base,” “reference” or original genetic background, by which subsequent strains that were derived, or were developed from said reference strain, are to be compared.

Once all SNPS in the diversity pool are identified, the present disclosure teaches methods of SNP swapping and screening methods to delineate (i.e. quantify and characterize) the effects (e.g. creation of a phenotype of interest) of SNPs individually and/or in groups.

In some embodiments, the SNP swapping methods of the present disclosure comprise the step of introducing one or more SNPs identified in a mutated strain (e.g., a strain from amongst S₂₋ₙGen₂₋ₙ) to a reference strain (S₁Gen₁) or wild-type strain (“wave up”).

In other embodiments, the SNP swapping methods of the present disclosure comprise the step of removing one or more SNPs identified in a mutated strain (e.g., a strain from amongst S₂₋ₙGen₂₋ₙ) (“wave down”).

In some embodiments, each generated strain comprising one or more SNP changes (either introducing or removing) is cultured and analyzed under one or more criteria of the present disclosure (e.g., production of a chemical or product of interest). Data from each of the analyzed host strains is associated, or correlated, with the particular SNP, or group of SNPs present in the host strain, and is recorded for future use. Thus, the present disclosure enables the creation of large and highly annotated HTP genetic design microbial strain libraries that are able to identify the effect of a given SNP on any number of microbial genetic or phenotypic traits of interest. The information stored in these HTP genetic design libraries informs the machine learning algorithms of the HTP genomic engineering platform and directs future iterations of the process, which ultimately leads to evolved microbial organisms that possess highly desirable properties/traits.

In some embodiments, the methods described herein can be carried out in a forward genetics procedure. For example, in some embodiments, the function and/or identity of genes that contain the SNPs or another type of genetic variations are not known, or are not considered in determining which SNP or other genetic variations are swapped or combined. Instead, combinations of genetic variations are made without consideration of known or predicted gene functions, but may be influenced by human or machine learning analysis of previous strain performance. Without wishing to be bound by any single theory, the present inventor believes that functionally agnostic screening is effective because it is not limited by human preconceptions and expectations. Thus, in some embodiments, the methods of the present disclosure allow for the discovery of valuable combinations of genetic variations that would not have been considered (and may even have been discouraged by) an “intelligent design” approach to genetic engineering.

In some embodiments, the method described herein can be carried out in a reverse genetics procedure. For example, in some embodiments, the function and/or identity of genes that contain the SNP or another type of genetic variations are already known and considered when the SNP or another type of genetic variations are swapped. For example, in some embodiments, genetic variations in genes involved in the synthesis, conversion, and/or degradation of a compound of interest (e.g., a spinosyn) are particularly selected and combined, with at least some hypothesis why such combinations may lead to improved strains with desired phenotypes. Such gene function and/or identity information include, but are not limited to, (1) genes in core biosynthetic pathway of a compound of interest, such as a spinosyn; (2) genes involved in precursor pool availability of a compound of interest, such as a gene directly involved in precursor synthesis or regulation of pool availability; (3) genes involved in cofactor utilization; (4) genes encoding with transcriptional regulators; (5) genes encoding transporters of nutrient availability; and (6) product exporters, etc.

In some embodiments, the method described herein can be carried out in a hybrid procedure, in which the function and/or identity of at least one gene or genetic variation is considered, while the function and/or identity of at least one gene that contains another genetic variation is not considered, when the genetic variations are combined.

Certain genes contain repeating segments of encoding DNA modules. For example, polyketides and non-ribosomal peptides are found to have modularity (see, US2017/0101659, incorporated by reference in its entirety). Functional protein domains in such proteins are arranged in a repetitive manner (module 1-module 2-module 3 . . . ) leads to repeating segments of DNA on the genome. In some embodiments, at least one genetic variation to be combined is not in a genomic region that contains repeating segments of encoding DNA modules. In some embodiments, the combination of genetic variations does not involve substitution, deletion, or addition of a repeated segment of encoding DNA module in such genes. The methods of the disclosure are able to perform targeted genomic editing not only in these areas of genomic modularity, but enable targeted genomic editing across the genome, in any genomic context. Consequently, the targeted genomic editing of the disclosure can edit the S. spinosa genome in any region, and is not bound to merely editing in areas having modularity.

3. Start/Stop Codon Exchanges: A Molecular Tool for the Derivation of Start/Stop Codon Microbial Strain Libraries

In some embodiments, the present disclosure teaches methods of swapping start and stop codon variants. For example, typical stop codons for S. cerevisiae and mammals are TAA (UAA) and TGA (UGA), respectively. The typical stop codon for monocotyledonous plants is TGA (UGA), whereas insects and E. coli commonly use TAA (UAA) as the stop codon (Dalphin et al. (1996) Nucl. Acids Res. 24: 216-218). In other embodiments, the present disclosure teaches use of the TAG (UAG) stop codons.

The present disclosure similarly teaches swapping start codons. In some embodiments, the present disclosure teaches use of the ATG (AUG) start codon utilized by most organisms (especially eukaryotes). In some embodiments, the present disclosure teaches that prokaryotes use ATG (AUG) the most, followed by GTG (GUG) and TTG (UUG).

In other embodiments, the present invention teaches replacing ATG start codons with TTG. In some embodiments, the present invention teaches replacing ATG start codons with GTG. In some embodiments, the present invention teaches replacing GTG start codons with ATG. In some embodiments, the present invention teaches replacing GTG start codons with TTG. In some embodiments, the present invention teaches replacing TTG start codons with ATG. In some embodiments, the present invention teaches replacing TTG start codons with GTG.

In other embodiments, the present invention teaches replacing TAA stop codons with TAG. In some embodiments, the present invention teaches replacing TAA stop codons with TGA. In some embodiments, the present invention teaches replacing TGA stop codons with TAA. In some embodiments, the present invention teaches replacing TGA stop codons with TAG. In some embodiments, the present invention teaches replacing TAG stop codons with TAA. In some embodiments, the present invention teaches replacing TAG stop codons with TGA.

4. Stop Swap: A Molecular Tool for the Derivation of Optimized Sequence Microbial Strain Libraries

In some embodiments, the present disclosure teaches methods of improving host cell productivity through the optimization of cellular gene transcription. Gene transcription is the result of several distinct biological phenomena, including transcriptional initiation (RNAp recruitment and transcriptional complex formation), elongation (strand synthesis/extension), and transcriptional termination (RNAp detachment and termination). Although much attention has been devoted to the control of gene expression through the transcriptional modulation of genes (e.g., by changing promoters, or inducing regulatory transcription factors), comparatively few efforts have been made towards the modulation of transcription via the modulation of gene terminator sequences.

The most obvious way that transcription impacts on gene expression levels is through the rate of Pol II initiation, which can be modulated by combinations of promoter or enhancer strength and trans-activating factors (Kadonaga, J T. 2004 “Regulation of RNA polymerase II transcription by sequence-specific DNA binding factors” Cell. 2004 Jan. 23; 116(2):247-57). In eukaryotes, elongation rate may also determine gene expression patterns by influencing alternative splicing (Cramer P. et al., 1997 “Functional association between promoter structure and transcript alternative splicing.” Proc Natl Acad Sci USA. 1997 Oct. 14; 94(21):11456-60). Failed termination on a gene can impair the expression of downstream genes by reducing the accessibility of the promoter to Pol II (Greger I H. et al., 2000 “Balancing transcriptional interference and initiation on the GAL7 promoter of Saccharomyces cerevisiae.” Proc Natl Acad Sci USA. 2000 Jul. 18; 97(15):8415-20). This process, known as transcriptional interference, is particularly relevant in lower eukaryotes, as they often have closely spaced genes.

Termination sequences can also affect the expression of the genes to which the sequences belong. For example, studies show that inefficient transcriptional termination in eukaryotes results in an accumulation of unspliced pre-mRNA (see West, S., and Proudfoot, N.J., 2009 “Transcriptional Termination Enhances Protein Expression in Human Cells” Mol Cell. 2009 Feb. 13; 33 (3-9); 354-364). Other studies have also shown that 3′ end processing, can be delayed by inefficient termination (West, S et al., 2008 “Molecular dissection of mammalian RNA polymerase II transcriptional termination.” Mol Cell. 2008 Mar. 14; 29(5):600-10). Transcriptional termination can also affect mRNA stability by releasing transcripts from sites of synthesis.

Termination of Transcription in Prokaryotes

In prokaryotes, two principal mechanisms, termed Rho-independent and Rho-dependent termination, mediate transcriptional termination. Rho-independent termination signals do not require an extrinsic transcription-termination factor, as formation of a stem-loop structure in the RNA transcribed from these sequences along with a series of Uridine (U) residues promotes release of the RNA chain from the transcription complex. Rho-dependent termination, on the other hand, requires a transcription-termination factor called Rho and cis-acting elements on the mRNA. The initial binding site for Rho, the Rho utilization (rut) site, is an extended (^{˜}70 nucleotides, sometimes 80-100 nucleotides) single-stranded region characterized by a high cytidine/low guanosine content and relatively little secondary structure in the RNA being synthesized, upstream of the actual terminator sequence. When a polymerase pause site is encountered, termination occurs, and the transcript is released by Rho's helicase activity.

Terminator Swapping (STOP Swap)

In some embodiments, the present disclosure teaches methods of selecting termination sequences (“terminators”) with optimal expression properties to produce beneficial effects on overall-host strain productivity.

For example, in some embodiments, the present disclosure teaches methods of identifying one or more terminators and/or generating variants of one or more terminators within a host cell, which exhibit a range of expression strengths (e.g. terminator ladders discussed infra). A particular combination of these identified and/or generated terminators can be grouped together as a terminator ladder, which is explained in more detail below.

The terminator ladder in question is then associated with a given gene of interest. Thus, if one has terminators T1-T8 (representing eight terminators that have been identified and/or generated to exhibit a range of expression strengths when combined with one or more promoters) and associates the terminator ladder with a single gene of interest in a host cell (i.e. genetically engineer a host cell with a given terminator operably linked to the 3′ end of to a given target gene), then the effect of each combination of the terminators can be ascertained by characterizing each of the engineered strains resulting from each combinatorial effort, given that the engineered host cells have an otherwise identical genetic background except the particular promoter(s) associated with the target gene. The resultant host cells that are engineered via this process form HTP genetic design libraries.

The HTP genetic design library can refer to the actual physical microbial strain collection that is formed via this process, with each member strain being representative of a given terminator operably linked to a particular target gene, in an otherwise identical genetic background, said library being termed a “terminator swap microbial strain library” or “STOP swap microbial strain library.”

Furthermore, the HTP genetic design library can refer to the collection of genetic perturbations—in this case a given terminator x operably linked to a given gene y—said collection being termed a “terminator swap library” or “STOP swap library.”

Further, one can utilize the same terminator ladder comprising promoters T₁-T₈ to engineer microbes, wherein each of the eight terminators is operably linked to 10 different gene targets. The result of this procedure would be 80 host cell strains that are otherwise assumed genetically identical, except for the particular terminators operably linked to a target gene of interest. These 80 host cell strains could be appropriately screened and characterized and give rise to another HTP genetic design library. The characterization of the microbial strains in the HTP genetic design library produces information and data that can be stored in any database, including without limitation, a relational database, an object-oriented database or a highly distributed NoSQL database. This data/information could include, for example, a given terminators' (e.g., T₁-T₈) effect when operably linked to a given gene target. This data/information can also be the broader set of combinatorial effects that result from operably linking two or more of promoters T₁-T₈ to a given gene target.

The aforementioned examples of eight terminators and 10 target genes is merely illustrative, as the concept can be applied with any given number of promoters that have been grouped together based upon exhibition of a range of expression strengths and any given number of target genes.

In summary, utilizing various terminators to modulate expression of various genes in an organism is a powerful tool to optimize a trait of interest. The molecular tool of terminator swapping, developed by the inventors, uses a ladder of terminator sequences that have been demonstrated to vary expression of at least one locus under at least one condition. This ladder is then systematically applied to a group of genes in the organism using high-throughput genome engineering. This group of genes is determined to have a high likelihood of impacting the trait of interest based on any one of a number of methods. These could include selection based on known function, or impact on the trait of interest, or algorithmic selection based on previously determined beneficial genetic diversity.

The resultant HTP genetic design microbial library of organisms containing a terminator sequence linked to a gene is then assessed for performance in a high-throughput screening model, and promoter-gene linkages which lead to increased performance are determined and the information stored in a database. The collection of genetic perturbations (i.e. given terminator x linked to a given gene y) form a “terminator swap library,” which can be utilized as a source of potential genetic alterations to be utilized in microbial engineering processing. Over time, as a greater set of genetic perturbations is implemented against a greater diversity of microbial backgrounds, each library becomes more powerful as a corpus of experimentally confirmed data that can be used to more precisely and predictably design targeted changes against any background of interest. That is in some embodiments, the present disclosures teaches introduction of one or more genetic changes into a host cell based on previous experimental results embedded within the meta data associated with any of the genetic design libraries of the invention.

Thus, in particular embodiments, terminator swapping is a multi-step process comprising:

1. Selecting a set of “x” terminators to act as a “ladder.” Ideally these terminators have been shown to lead to highly variable expression across multiple genomic loci, but the only requirement is that they perturb gene expression in some way.

2. Selecting a set of “n” genes to target. This set can be every ORF in a genome, or a subset of ORFs. The subset can be chosen using annotations on ORFs related to function, by relation to previously demonstrated beneficial perturbations (previous promoter swaps, STOP swaps, or SNP swaps), by algorithmic selection based on epistatic interactions between previously generated perturbations, other selection criteria based on hypotheses regarding beneficial ORF to target, or through random selection. In other embodiments, the “n” targeted genes can comprise non-protein coding genes, including non-coding RNAs.

3. High-throughput strain engineering to rapidly and in parallel carry out the following genetic modifications: When a native terminator exists at the 3′ end of target gene n and its sequence is known, replace the native terminator with each of the x terminators in the ladder. When the native terminator does not exist, or its sequence is unknown, insert each of the x terminators in the ladder after the gene stop codon.

In this way a “library” (also referred to as a HTP genetic design library) of strains is constructed, wherein each member of the library is an instance of x terminator linked to n target, in an otherwise identical genetic context. As previously described, combinations of terminators can be inserted, extending the range of combinatorial possibilities upon which the library is constructed.

4. High-throughput screening of the library of strains in a context where their performance against one or more metrics is indicative of the performance that is being optimized.

This foundational process can be extended to provide further improvements in strain performance by, inter alia: (1) Consolidating multiple beneficial perturbations into a single strain background, either one at a time in an interactive process, or as multiple changes in a single step. Multiple perturbations can be either a specific set of defined changes or a partly randomized, combinatorial library of changes. For example, if the set of targets is every gene in a pathway, then sequential regeneration of the library of perturbations into an improved member or members of the previous library of strains can optimize the expression level of each gene in a pathway regardless of which genes are rate limiting at any given iteration; (2) Feeding the performance data resulting from the individual and combinatorial generation of the library into an algorithm that uses that data to predict an optimum set of perturbations based on the interaction of each perturbation; and (3) Implementing a combination of the above two approaches.

The approach is exemplified in the present disclosure with industrial microorganisms, but is applicable to any organism where desired traits can be identified in a population of genetic mutants. For example, this could be used for improving the performance of CHO cells, yeast, insect cells, algae, as well as multi-cellular organisms, such as plants.

In some embodiments, provided are a set of terminator sequences that can be used to create terminator swap library according to the present disclosure. This set of terminator sequence includes those described in Table 3, and any functional variants thereof, such as terminator sequences having at least 70%, 75%, 80%, 85%, 90%, 95%, 99% or more identity to SEQ ID No. 70 to SEQ ID No. 80.

5. Transposon Mutagenesis Diversity Libraries: A Molecular Tool for the Derivation of Transposon Mutagenesis Diversity Libraries

Certain tools described in the present disclosure concerns existing polymorphs of genes in microbial strains, but do not create novel mutations that may be useful for improving performance of the microbial strains. The present disclosure teaches a transposon mutagenesis system that randomly create mutations that can be further screened for those leading to improved features of the host strains, which in turn cause beneficial effects on overall-host strain phenotype (e.g., yield or productivity).

For example, in some embodiments, the present disclosure teaches methods of generating and identifying mutations within a host cell, which exhibit a range of expression profiles of one or more genes in the host cell. Any particular mutation generated in this process can be grouped together as a transposon mutagenesis diversity library, which is explained in more detail below.

The resultant microbes that are engineered via this process form HTP genetic design libraries.

The HTP genetic design library can refer to the actual physical microbial strain collection that is formed via this process, with each member strain being representative of a given mutation created by transposon mutagenesis, in an otherwise identical genetic background, said library being termed a “transposon mutagenesis diversity library.”

Furthermore, the HTP genetic design library can refer to the collection of genetic perturbations—in this case a given mutation created by transposon mutagenesis.

Further, also provided are microbes that are otherwise assumed genetically identical, except for the particular mutation created by transposon mutagenesis. These microbes could be appropriately screened and characterized and give rise to another HTP genetic design library. The characterization of the microbial strains in the HTP genetic design library produces information and data that can be stored in any data storage construct, including a relational database, an object-oriented database or a highly distributed NoSQL database. This data/information could be, for example, a mutation's effect on host cell growth or production of a molecule in the host cell. This data/information can also be the broader set of combinatorial effects that result from two or more mutations.

The aforementioned examples of mutations created by transposon mutagenesis is merely illustrative, as the concept can be applied with any given number of mutations that have been grouped together based upon exhibition of a range of expression profile and their impacts on any given number of genes. Persons having skill in the art will also recognize the ability to consolidate a mutation created by transposon mutagenesis with any other mutations. Thus, in some embodiments, the present disclosure teaches libraries in which 1, 2, 3, 4, 5, 6, 7, 8, 9, 10, 20, 30, 40, 50, 60, 70, 80, 90, 100, 200, 300, 400, 500, 600, 700, 800, 900, 1000, or more mutations are consolidated.

In summary, utilizing various mutations created by transposon mutagenesis in an organism is a powerful tool to optimize a trait of interest. The molecular tool of transposon mutagenesis diversity libraries, developed by the inventors, uses a collection of mutations having vary expression profile. This collection is then systematically applied in the organism using high-throughput genome engineering. This group of mutations is determined to have a high likelihood of impacting the trait of interest based on any one of a number of methods. In some embodiments, the libraries contain saturated number of mutations (e.g., in theory each gene in the genome of the microorganism is hit at least once). In some embodiments, genomic locations of the mutations in the transposon mutagenesis libraries are not determined, thus the libraries contains randomly distributed mutations in the genome of the microorganisms. In some embodiments, mutations in the transposon mutagenesis libraries are selected based on associated phenotypes. In some embodiments, mutations in the transposon mutagenesis libraries are characterized and the genomic location of the mutations are determined, and genes disrupted by the mutations are identified. These could include selection based on known function, or impact on the trait of interest, or algorithmic selection based on previously determined beneficial genetic diversity. In some embodiments, the selection of mutations can include all the genes in a given host. In other embodiments, the selection of mutations can be a subset of all genes in a given host, chosen randomly. In other embodiments, the selection of mutations can be a subset of all genes involved in the synthesis of a given molecule, such as a spinosyn in Saccharopolyspora spp.

The resultant HTP genetic design microbial strain library of organisms containing mutations created by transposon mutagenesis is then assessed for performance in a high-throughput screening model, and mutations which lead to increased performance are determined and the information stored in a database. The collection of genetic perturbations (i.e. mutations) form a “transposon mutagenesis library,” which can be utilized as a source of potential genetic alterations to be utilized in microbial engineering processing. Over time, as a greater set of genetic perturbations is implemented against a greater diversity of host cell backgrounds, each library becomes more powerful as a corpus of experimentally confirmed data that can be used to more precisely and predictably design targeted changes against any background of interest.

In some embodiments, the transposon mutagenesis diversity library of the present disclosure can be used to identify optimum expression of a gene target. In some embodiments, the goal may be to increase activity of a target gene to reduce bottlenecks in a metabolic or genetic pathway. In other embodiments, the goal may be to reduce the activity of the target gene to avoid unnecessary energy expenditures in the host cell, when expression of said target gene is not required.

Thus, in particular embodiments, the method of using a transposon mutagenesis diversity library is a multi-step process comprising:

1. Selecting a transposon system for mutagenesis and applying the system in a given microbial strain to generate mutations caused by the transposon. Ideally the system is shown to lead to random integration of transposon into the genome of a selected microbial strain, such as a Saccharopolyspora strain. Such integration perturbs gene expression in some way.

2. High-throughput strain engineering to rapidly select strains having integrated transposon in its genome. In this way a “library” (also referred to as a HTP genetic design library) of strains is constructed, wherein each member of the library is a strain comprising a transposon mutation, otherwise identical genetic context. As previously described combinations of mutations can be consolidated, extending the range of combinatorial possibilities upon which the library is constructed.

3. High-throughput screening of the library of strains in a context where their performance against one or more metrics is indicative of the performance that is being optimized.

This foundational process can be extended to provide further improvements in strain performance by, inter alia: (1) Consolidating multiple beneficial perturbations (mutations) into a single strain background, either one at a time in an iterative process, or as multiple changes in a single step. Multiple perturbations (mutations) can be either a specific set of defined changes or a partly randomized, combinatorial library of changes, regardless of the gene function that has been modified by the mutations; (2) Feeding the performance data resulting from the individual and combinatorial generation of the library into an algorithm that uses that data to predict an optimum set of perturbations based on the interaction of each perturbation; and (3) Implementing a combination of the above two approaches.

In some embodiments, the transposase is functional in Saccharopolyspora spp. In some embodiments, the transpose is derived from EZ-Tn5 transposon system. In some embodiments, the DNA payload sequence is flanked by mosaic elements (ME) that can be recognized by said transposase. In some embodiments, the DNA payload can be a loss-of-function (LoF) transposon, or a gain-of-function (GoF) transposon.

In some embodiments, the DNA payload comprises a selection marker. In some embodiments, selectable markers that can be used in the transposon mutagenesis process of the present disclosure include, but are not limited to aac(3)IV conferring resistance to Apramycin (SEQ ID No. 151), aacC1 conferring resistance to Gentamycin (SEQ ID No. 152), acC8 conferring resistance to Neomycin B (SEQ ID No. 153), aadA conferring resistance to Spectinomycin/Streptomycin (SEQ ID No. 154), ble conferring resistance to Bleomycin (SEQ ID No. 155), cat conferring resistance to Chloramphenicol (SEQ ID No. 156), ermE conferring resistance to Erythromycin (SEQ ID No. 157), hyg onferring resistance to Hygromycin (SEQ ID No. 158), and neo conferring resistance to Kanamycin (SEQ ID No. 159). In some embodiments, the selection marker is used to screen for Saccharopolyspora cells containing the transposon.

In some embodiments, the DNA payload comprises a counter-selection marker. In some embodiments, the counter-selection marker is used to facilitate loop-out of a DNA payload containing the selectable marker. In some embodiments, counter-selection markers that can be used in the transposon mutagenesis process of the present disclosure include, but are not limited to SEQ ID No. 160 (amdSYM), SEQ ID No. 161 (tetA), SEQ ID No. 162 (lacY), SEQ ID No. 163 (sacB), SEQ ID No. 164 (pheS, S. erythraea), SEQ ID No. 165 (pheS, Corynebacterium).

In some embodiments, the methods of the disclosure are able to perform targeted genomic editing not only in these areas of genomic modularity, but enable targeted genomic editing across the genome, in any genomic context. Consequently, the targeted genomic editing of the disclosure can edit the S. spinosa genome in any region, and is not bound to merely editing in areas having modularity.

In some embodiments, the GoF transposon comprises a GoF element. In some embodiments, the GoF transposon comprises a promoter sequence and/or a solubility tag sequence (e.g., SEQ ID No. 166).

In some embodiments, the transposon mutagenesis library of the present disclosure has 95% confidence in hitting every gene at least once. In some embodiments, such library is obtained by screening a number of isolates that is approximately 3× the number of genes in the organism. For S. spinosa, which contains ˜8000 annotated genes, we expect a mutagenesis library size of ˜24,000 members to cover the genome.

In some embodiments, high-throughput screening of the transposon mutagenesis library of strains produces a collection of strains having improved performance compared to a reference strain. In some embodiments, mutations in these collected strains due to the transposon mutagenesis which leads to the improved performance of these collected strains are consolidated to produce new strains with enriched targets of interest. In some embodiments, such strains with enriched targets of interest can be combined with other strains of the present disclosure (e.g., strains with improved performance in the SNP Swap or Promoter Swap libraries) for further directed strain engineering.

6. Ribosomal Binding Site (RBS) Diversity Library: A Molecular Tool for the Derivation of RBS Microbial Strain Libraries

In some embodiments, the present disclosure teaches methods of selecting ribosomal binding sites (RBSs) with optimal expression properties to produce beneficial effects on overall-host strain phenotype (e.g., yield or productivity).

For example, in some embodiments, the present disclosure teaches methods of identifying one or more RBSs and/or generating variants of one or more RBSs within a host cell, which exhibit a range of expression strengths (e.g. RBS ladders discussed infra), or superior regulatory properties (e.g., tighter regulatory control for selected genes). A particular combination of these identified and/or generated RBSs can be grouped together as a RBS ladder, which is explained in more detail below.

The RBS ladder in question in some embodiments is then associated with a given gene of interest. Thus, if one has RBS1 to RBS31 (representing 31 RBSs that have been identified and/or generated to exhibit a range of expression strengths, SEQ ID No. 97 to SEQ ID No. 127) and associates the RBS ladder with a single gene of interest in a microbe (i.e. genetically engineer a microbe with a given RBS operably linked to a given target gene), then the effect of each combination of the 31 RBS can be ascertained by characterizing each of the engineered strains resulting from each combinatorial effort, given that the engineered microbes have an otherwise identical genetic background except the particular RBS(s) associated with the target gene.

The resultant microbes that are engineered via this process form HTP genetic design libraries.

The HTP genetic design library can refer to the actual physical microbial strain collection that is formed via this process, with each member strain being representative of a given RBS operably linked to a particular target gene, in an otherwise identical genetic background, said library being termed a “RBS library.”

Furthermore, the HTP genetic design library can refer to the collection of genetic perturbations—in this case a given RBS x operably linked to a given gene y (and optionally also linked to a given promoter z).

Further, one can utilize the same RBS ladder comprising RBSs in Table 11 to engineer microbes, wherein each of the RBS is operably linked to different gene targets. The result of this procedure would be microbes that are otherwise assumed genetically identical, except for the particular RBSs operably linked to a target gene of interest. These microbes could be appropriately screened and characterized and give rise to another HTP genetic design library. The characterization of the microbial strains in the HTP genetic design library produces information and data that can be stored in any data storage construct, including a relational database, an object-oriented database or a highly distributed NoSQL database. This data/information could be, for example, a given RBS' effect when operably linked to a given gene target. This data/information can also be the broader set of combinatorial effects that result from operably linking two or more of RBS of the present disclosure to a given gene target.

The aforementioned examples of RBSs and target genes is merely illustrative, as the concept can be applied with any given number of RBSs that have been grouped together based upon exhibition of a range of expression strengths and any given number of target genes. Persons having skill in the art will also recognize the ability to operably link two or more RBSs in front of any gene target. Thus, in some embodiments, the present disclosure teaches RBS libraries in which 1, 2, 3 or more RBSs from a RBS ladder are operably linked to one or more genes.

In summary, utilizing various RBSs to drive expression of various genes in an organism is a powerful tool to optimize a trait of interest. The molecular tool of RBS libraries, developed by the inventors, uses a ladder of RBS sequences that have been demonstrated to vary expression of at least one locus under at least one condition. This ladder is then systematically applied to a group of genes in the organism using high-throughput genome engineering. This group of genes is determined to have a high likelihood of impacting the trait of interest based on any one of a number of methods. These could include selection based on known function, or impact on the trait of interest, or algorithmic selection based on previously determined beneficial genetic diversity. In some embodiments, the selection of genes can include all the genes in a given host. In other embodiments, the selection of genes can be a subset of all genes in a given host, chosen randomly.

The resultant HTP genetic design microbial strain library of organisms containing a RBS sequence linked to a gene is then assessed for performance in a high-throughput screening model, and RBS-gene linkages which lead to increased performance are determined and the information stored in a database. The collection of genetic perturbations (i.e. given RBS x operably linked to a given gene y) form a “RBS diversity library,” which can be utilized as a source of potential genetic alterations to be utilized in microbial engineering processing. Over time, as a greater set of genetic perturbations is implemented against a greater diversity of host cell backgrounds, each library becomes more powerful as a corpus of experimentally confirmed data that can be used to more precisely and predictably design targeted changes against any background of interest.

Transcription levels of genes in an organism are a key point of control for affecting organism behavior. Transcription is tightly coupled to translation (protein expression), and which proteins are expressed in what quantities determines organism behavior. Cells express thousands of different types of proteins, and these proteins interact in numerous complex ways to create function. By varying the expression levels of a set of proteins systematically, function can be altered in ways that, because of complexity, are difficult to predict. Some alterations may increase performance, and so, coupled to a mechanism for assessing performance, this technique allows for the generation of organisms with improved function.

In the context of a small molecule synthesis pathway, enzymes interact through their small molecule substrates and products in a linear or branched chain, starting with a substrate and ending with a small molecule of interest. Because these interactions are sequentially linked, this system exhibits distributed control, and increasing the expression of one enzyme can only increase pathway flux until another enzyme becomes rate limiting.

Metabolic Control Analysis (MCA) is a method for determining, from experimental data and first principles, which enzyme or enzymes are rate limiting. MCA is limited however, because it requires extensive experimentation after each expression level change to determine the new rate limiting enzyme. RBS libraries are advantageous in this context, because through the application of a RBS ladder to each enzyme in a pathway, the limiting enzyme is found, and the same thing can be done in subsequent rounds to find new enzymes that become rate limiting. Further, because the read-out on function is better production of the small molecule of interest, the experiment to determine which enzyme is limiting is the same as the engineering to increase production, thus shortening development time. In some embodiments the present disclosure teaches the application of RBS libraries to genes encoding individual subunits of multi-unit enzymes. In yet other embodiments, the present disclosure teaches methods of applying RBS library techniques to genes responsible for regulating individual enzymes, or whole biosynthetic pathways.

In some embodiments, the RBS libraries of the present disclosure can be used to identify optimum expression of a selected gene target. In some embodiments, the goal of the RBS libraries may be to increase expression of a target gene to reduce bottlenecks in a metabolic or genetic pathway. In other embodiments, the goal of the RBS libraries may be to reduce the expression of the target gene to avoid unnecessary energy expenditures in the host cell, when expression of said target gene is not required.

In the context of other cellular systems like transcription, transport, or signaling, various rational methods can be used to try and find out, a priori, which proteins are targets for expression change and what that change should be. These rational methods reduce the number of perturbations that must be tested to find one that improves performance, but they do so at significant cost. Gene deletion studies identify proteins whose presence is critical for a particular function, and important genes can then be over-expressed. Due to the complexity of protein interactions, this is often ineffective at increasing performance. Different types of models have been developed that attempt to describe, from first principles, transcription or signaling behavior as a function of protein levels in the cell. These models often suggest targets where expression changes might lead to different or improved function. The assumptions that underlie these models are simplistic and the parameters difficult to measure, so the predictions they make are often incorrect, especially for non-model organisms. With both gene deletion and modeling, the experiments required to determine how to affect a certain gene are different than the subsequent work to make the change that improves performance. RBS library method sidesteps these challenges, because the constructed strain that highlights the importance of a particular perturbation is also, already, the improved strain.

Thus, in particular embodiments, the method of using RBS libraries is a multi-step process comprising:

1. Selecting a set of “x” RBSs to act as a “ladder.” Ideally these RBSs have been shown to lead to highly variable expression across multiple genomic loci, but the only requirement is that they perturb gene expression in some way.

2. Selecting a set of “n” genes to target. This set can be every open reading frame (ORF) in a genome, or a subset of ORFs. The subset can be chosen using annotations on ORFs related to function, by relation to previously demonstrated beneficial perturbations (previous RBS collections or previous SNP swaps), by algorithmic selection based on epistatic interactions between previously generated perturbations, other selection criteria based on hypotheses regarding beneficial ORF to target, or through random selection. In other embodiments, the “n” targeted genes can comprise non-protein coding genes, including non-coding RNAs.

3. High-throughput strain engineering to rapidly-and in some embodiments, in parallel-carry out the following genetic modifications: When a native RBS exists in front of target gene n and its sequence is known, replace the native RBS with each of the x RBSs in the ladder. When the native RBS does not exist, or its sequence is unknown, insert each of the x RBSs in the ladder in front of gene n. In this way a “library” (also referred to as a HTP genetic design library) of strains is constructed, wherein each member of the library is an instance of x RBS operably linked to n target, in an otherwise identical genetic context. As previously described combinations of RBSs can be inserted, extending the range of combinatorial possibilities upon which the library is constructed.

4. High-throughput screening of the library of strains in a context where their performance against one or more metrics is indicative of the performance that is being optimized.

This foundational process can be extended to provide further improvements in strain performance by, inter alia: (1) Consolidating multiple beneficial perturbations into a single strain background, either one at a time in an interactive process, or as multiple changes in a single step. Multiple perturbations can be either a specific set of defined changes or a partly randomized, combinatorial library of changes. For example, if the set of targets is every gene in a pathway, then sequential regeneration of the library of perturbations into an improved member or members of the previous library of strains can optimize the expression level of each gene in a pathway regardless of which genes are rate limiting at any given iteration; (2) Feeding the performance data resulting from the individual and combinatorial generation of the library into an algorithm that uses that data to predict an optimum set of perturbations based on the interaction of each perturbation; and (3) Implementing a combination of the above two approaches.

The approach is exemplified in the present disclosure with industrial microorganisms, but is applicable to any organism where desired traits can be identified in a population of genetic mutants. For example, this could be used for improving the performance of CHO cells, yeast, insect cells, algae, as well as multi-cellular organisms, such as plants.

In some embodiments, RBS libraries of the present disclosure can be used as a source of genetic diversity. In some embodiments, RBS ladders of the present disclosure when introduced into Saccharopolyspora strains leads to the improved performance of the strains. Such improved strains can be further consolidated with other strains bearing additional genetic diversity of the present disclosure (e.g., strains with improved performance in the SNP Swap or Promoter Swap libraries), to produce new strains with enriched targets of interest. In some embodiments, such strains with enriched targets of interest can be used for further directed strain engineering.

7. Anti-Metabolite Selection/Fermentation Product Resistance Libraries: A Molecular Tool for the Derivation of Polymorph Microbial Strain Libraries

In order to improve production of desired compounds by microbes it is often needed to overcome the end-product inhibition issue. Microbes produce a variety of compounds as a part of the fermentation process. Sometimes the accumulation of such compounds severely inhibits the growth and physiology of the microbes. To improve fermentation and lengthen the time during which the microbe can synthesize the desired metabolites, one has to overcome a) the potential toxicity of the end product, and b) feed-back inhibition of molecular pathways needed for the formation of the desired end-product.

(a) In some embodiments, the present disclosure teaches methods of generating and identifying mutations within a host cell, which exhibit a range of expression profiles of one or more genes in the host cell, particularly mutations that lead to improved resistance to a give metabolite in the host cell or fermentation product, thus improving the performance of the host cell. Any particular mutation identified in this process can be grouped together as an anti-metabolite selection/fermentation product resistance library, which is explained in more detail below.

The resultant microbes that are engineered via this process form HTP genetic design libraries.

The HTP genetic design library can refer to the actual physical microbial strain collection that is formed via this process, with each member strain being representative of a given mutation identified in the process, in an otherwise identical genetic background, said library being termed an “anti-metabolite selection/fermentation product resistance library.”

Furthermore, the HTP genetic design library can refer to the collection of genetic perturbations—in this case a given mutation created by the process described herein.

Further, also provided are microbes that are otherwise assumed genetically identical, except for the particular mutation causing resistance to a given metabolite or a fermentation product. These microbes could be appropriately screened and characterized and give rise to another HTP genetic design library. The characterization of the microbial strains in the HTP genetic design library produces information and data that can be stored in any data storage construct, including a relational database, an object-oriented database or a highly distributed NoSQL database. This data/information could be, for example, a mutation's effect on host cell growth or production of a molecule in the host cell. This data/information can also be the broader set of combinatorial effects that result from two or more mutations.

The aforementioned examples of mutations created by the process is merely illustrative, as the concept can be applied with any given number of mutations that have been grouped together based upon exhibition of a range of expression profile and their impacts on any given number of genes. Persons having skill in the art will also recognize the ability to consolidate a mutation created by the process described herein with any other mutations. Thus, in some embodiments, the present disclosure teaches libraries in which 1, 2, 3, 4, 5, 6, 7, 8, 9, 10, 20, 30, 40, 50, 60, 70, 80, 90, 100, 200, 300, 400, 500, 600, 700, 800, 900, 1000, or more mutations are consolidated.

In summary, utilizing various mutations that cause resistance to a given metabolite or a fermentation product in an organism is a powerful tool to optimize a trait of interest. The molecular tool uses a collection of mutations resistance to a given metabolite or a fermentation product. In some embodiments, such mutations lead to improved performance in the strains, such as increased yield or production of one or more given molecule, such as a spinosyn. This collection is then systematically applied in the organism using high-throughput genome engineering. This group of mutations is determined to have a high likelihood of impacting the trait of interest based on any one of a number of methods. These could include selection based on known function, or impact on the trait of interest, or algorithmic selection based on previously determined beneficial genetic diversity. In some embodiments, the selection of mutations can include all the genes in a given host. In other embodiments, the selection of mutations can be a subset of all genes in a given host, chosen randomly. In other embodiments, the selection of mutations can be a subset of all genes involved in the synthesis of a given molecule, such as a spinosyn in Saccharopolyspora spp.

The resultant HTP genetic design microbial strain library of organisms containing mutations that cause resistance to a given metabolite or a fermentation product is then assessed for performance in a high-throughput screening model, and mutations which lead to increased performance are determined and the information stored in a database. The collection of genetic perturbations (i.e. mutations) form a “anti-metabolite selection/fermentation product resistance library,” which can be utilized as a source of potential genetic alterations to be utilized in microbial engineering processing. Over time, as a greater set of genetic perturbations is implemented against a greater diversity of host cell backgrounds, each library becomes more powerful as a corpus of experimentally confirmed data that can be used to more precisely and predictably design targeted changes against any background of interest.

In some embodiments, the anti-metabolite selection/fermentation product resistance diversity libraries of the present disclosure can be used to identify optimum expression of a gene target. In some embodiments, the goal may be to increase activity of a target gene to reduce bottlenecks in a metabolic or genetic pathway. In other embodiments, the goal may be to reduce the activity of the target gene to avoid unnecessary energy expenditures in the host cell, when expression of said target gene is not required.

Thus, in particular embodiments, a method of applying anti-metabolite selection/fermentation product resistance library is a multi-step process comprising:

1. High-throughput strain engineering to rapidly select strains that are resistant to one or more given metabolite or fermentation products in the host strain. Ideally the system is shown to identify strains with all types of polymorphs, regardless whether the polymorphs are related to synthesis of the given metabolite or fermentation product.

2. High-throughput strain engineering to rapidly select strains that indeed have improved performance (e.g., increased yield or production of a given metabolite or a fermentation product). In this way a “library” (also referred to as a HTP genetic design library) of strains is constructed, wherein each member of the library is a strain comprising one or more beneficial polymorphs, otherwise identical genetic context. As previously described combinations of polymorphs can be consolidated, extending the range of combinatorial possibilities upon which the library is constructed.

3. High-throughput screening of the library of strains in a context where their performance against one or more metrics is indicative of the performance that is being optimized.

In some embodiments, the method also comprises the step of determining the strategy for the initial selecting step 1 as described above, such as selecting for preferred metabolite/fermentation product that cause cell growth inhibition, proper concentration of metabolite/fermentation product.

In some embodiments, anti-metabolite selection/fermentation product resistance libraries of the present disclosure can be used as a source of genetic diversity. In some embodiments, mutations that lead to improved resistance to a metabolite or a fermentation product identified by the methods of the present disclosure lead to the improved performance of the strains. Such improved strains can be further consolidated with other strains bearing additional genetic diversity of the present disclosure (e.g., strains with improved performance in the SNP Swap or Promoter Swap libraries, or the transposon mutagenesis libraries), to produce new strains with enriched targets of interest. In some embodiments, such strains with enriched targets of interest can be used for further directed strain engineering.

8. Sequence Optimization: A Molecular Tool for the Derivation of Optimized Sequence Microbial Strain Libraries

In one embodiment, the methods of the provided disclosure comprise codon optimizing one or more genes expressed by the host organism. Methods for optimizing codons to improve expression in various hosts are known in the art and are described in the literature (see U.S. Pat. App. Pub. No. 2007/0292918, incorporated herein by reference in its entirety). Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence.

Protein expression is governed by a host of factors including those that affect transcription, mRNA processing, and stability and initiation of translation. Optimization can thus address any of a number of sequence features of any particular gene. As a specific example, a rare codon induced translational pause can result in reduced protein expression. A rare codon induced translational pause includes the presence of codons in the polynucleotide of interest that are rarely used in the host organism may have a negative effect on protein translation due to their scarcity in the available tRNA pool.

Alternate translational initiation also can result in reduced heterologous protein expression. Alternate translational initiation can include a synthetic polynucleotide sequence inadvertently containing motifs capable of functioning as a ribosome binding site (RBS). These sites can result in initiating translation of a truncated protein from a gene-internal site. One method of reducing the possibility of producing a truncated protein, which can be difficult to remove during purification, includes eliminating putative internal RBS sequences from an optimized polynucleotide sequence.

Repeat-induced polymerase slippage can result in reduced heterologous protein expression. Repeat-induced polymerase slippage involves nucleotide sequence repeats that have been shown to cause slippage or stuttering of DNA polymerase which can result in frameshift mutations. Such repeats can also cause slippage of RNA polymerase. In an organism with a high G+C content bias, there can be a higher degree of repeats composed of G or C nucleotide repeats. Therefore, one method of reducing the possibility of inducing RNA polymerase slippage, includes altering extended repeats of G or C nucleotides.

Interfering secondary structures also can result in reduced heterologous protein expression. Secondary structures can sequester the RBS sequence or initiation codon and have been correlated to a reduction in protein expression. Stemloop structures can also be involved in transcriptional pausing and attenuation. An optimized polynucleotide sequence can contain minimal secondary structures in the RBS and gene coding regions of the nucleotide sequence to allow for improved transcription and translation.

For example, the optimization process can begin by identifying the desired amino acid sequence to be expressed by the host. From the amino acid sequence a candidate polynucleotide or DNA sequence can be designed. During the design of the synthetic DNA sequence, the frequency of codon usage can be compared to the codon usage of the host expression organism and rare host codons can be removed from the synthetic sequence. Additionally, the synthetic candidate DNA sequence can be modified in order to remove undesirable enzyme restriction sites and add or remove any desired signal sequences, linkers or untranslated regions. The synthetic DNA sequence can be analyzed for the presence of secondary structure that may interfere with the translation process, such as G/C repeats and stem-loop structures.

9. Epistasis Mapping—a Predictive Analytical Tool Enabling Beneficial Genetic Consolidations

In some embodiments, the present disclosure teaches epistasis mapping methods for predicting and combining beneficial genetic alterations into a host cell. The genetic alterations may be created by any of the aforementioned HTP molecular tool sets (e.g., promoter swaps, SNP swaps, start/stop codon exchanges, sequence optimization) and the effect of those genetic alterations would be known from the characterization of the derived HTP genetic design microbial strain libraries. Thus, as used herein, the term epistasis mapping includes methods of identifying combinations of genetic alterations (e.g., beneficial SNPs or beneficial promoter/target gene associations) that are likely to yield increases in host performance.

In embodiments, the epistasis mapping methods of the present disclosure are based on the idea that the combination of beneficial mutations from two different functional groups is more likely to improve host performance, as compared to a combination of mutations from the same functional group. See, e.g., Costanzo, The Genetic Landscape of a Cell, Science, Vol. 327, Issue 5964, Jan. 22, 2010, pp. 425-431 (incorporated by reference herein in its entirety).

Mutations from the same functional group are more likely to operate by the same mechanism, and are thus more likely to exhibit negative or neutral epistasis on overall host performance. In contrast, mutations from different functional groups are more likely to operate by independent mechanisms, which can lead to improved host performance and in some instances synergistic effects.

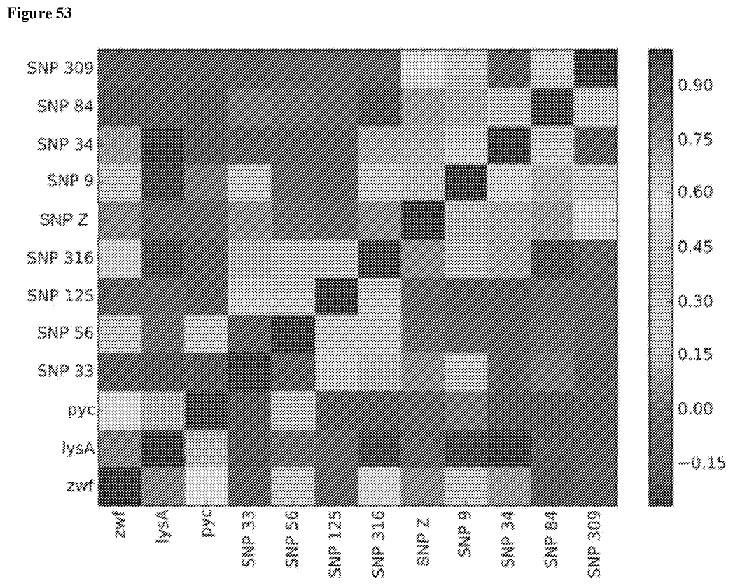

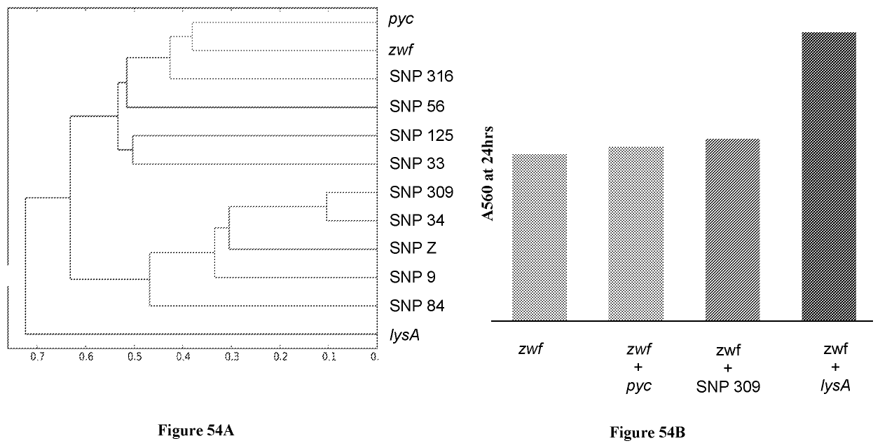

Thus, in some embodiments, the present disclosure teaches methods of analyzing SNP mutations to identify SNPs predicted to belong to different functional groups. In some embodiments, SNP functional group similarity is determined by computing the cosine similarity of mutation interaction profiles (similar to a correlation coefficient, see FIG. 54A). The present disclosure also illustrates comparing SNPs via a mutation similarity matrix (see FIG. 53) or dendrogram (see FIG. 54A).

Thus, the epistasis mapping procedure provides a method for grouping and/or ranking a diversity of genetic mutations applied in one or more genetic backgrounds for the purposes of efficient and effective consolidations of said mutations into one or more genetic backgrounds.

In aspects, consolidation is performed with the objective of creating novel strains which are optimized for the production of target biomolecules. Through the taught epistasis mapping procedure, it is possible to identify functional groupings of mutations, and such functional groupings enable a consolidation strategy that minimizes undesirable epistatic effects.

As previously explained, the optimization of microbes for use in industrial fermentation is an important and difficult problem, with broad implications for the economy, society, and the natural world. Traditionally, microbial engineering has been performed through a slow and uncertain process of random mutagenesis. Such approaches leverage the natural evolutionary capacity of cells to adapt to artificially imposed selection pressure. Such approaches are also limited by the rarity of beneficial mutations, the ruggedness of the underlying fitness landscape, and more generally underutilize the state of the art in cellular and molecular biology.

Modern approaches leverage new understanding of cellular function at the mechanistic level and new molecular biology tools to perform targeted genetic manipulations to specific phenotypic ends. In practice, such rational approaches are confounded by the underlying complexity of biology. Causal mechanisms are poorly understood, particularly when attempting to combine two or more changes that each has an observed beneficial effect. Sometimes such consolidations of genetic changes yield positive outcomes (measured by increases in desired phenotypic activity), although the net positive outcome may be lower than expected and in some cases higher than expected. In other instances, such combinations produce either net neutral effect or a net negative effect. This phenomenon is referred to as epistasis, and is one of the fundamental challenges to microbial engineering (and genetic engineering generally).

As aforementioned, the present HTP genomic engineering platform solves many of the problems associated with traditional microbial engineering approaches. The present HTP platform uses automation technologies to perform hundreds or thousands of genetic mutations at once. In particular aspects, unlike the rational approaches described above, the disclosed HTP platform enables the parallel construction of thousands of mutants to more effectively explore large subsets of the relevant genomic space, as disclosed in U.S. application Ser. No. 15/140,296, entitled Microbial Strain Design System And Methods For Improved Large-Scale Production Of Engineered Nucleotide Sequences, incorporated by reference herein in its entirety. By trying “everything,” the present HTP platform sidesteps the difficulties induced by our limited biological understanding.

However, at the same time, the present HTP platform faces the problem of being fundamentally limited by the combinatorial explosive size of genomic space, and the effectiveness of computational techniques to interpret the generated data sets given the complexity of genetic interactions. Techniques are needed to explore subsets of vast combinatorial spaces in ways that maximize non-random selection of combinations that yield desired outcomes.

Somewhat similar HTP approaches have proved effective in the case of enzyme optimization. In this niche problem, a genomic sequence of interest (on the order of 1000 bases), encodes a protein chain with some complicated physical configuration. The precise configuration is determined by the collective electromagnetic interactions between its constituent atomic components. This combination of short genomic sequence and physically constrained folding problem lends itself specifically to greedy optimization strategies. That is, it is possible to individually mutate the sequence at every residue and shuffle the resulting mutants to effectively sample local sequence space at a resolution compatible with the Sequence Activity Response modeling.

However, for full genomic optimizations for biomolecules, such residue-centric approaches are insufficient for some important reasons. First, because of the exponential increase in relevant sequence space associated with genomic optimizations for biomolecules. Second, because of the added complexity of regulation, expression, and metabolic interactions in biomolecule synthesis. The present inventors have solved these problems via the taught epistasis mapping procedure.

The taught method for modeling epistatic interactions, between a collection of mutations for the purposes of more efficient and effective consolidation of said mutations into one or more genetic backgrounds, is groundbreaking and highly needed in the art.

When describing the epistasis mapping procedure, the terms “more efficient” and “more effective” refers to the avoidance of undesirable epistatic interactions among consolidation strains with respect to particular phenotypic objectives.

As the process has been generally elaborated upon above, a more specific workflow example will now be described.

First, one begins with a library of M mutations and one or more genetic backgrounds (e.g., parent bacterial strains). Neither the choice of library nor the choice of genetic backgrounds is specific to the method described here. But in a particular implementation, a library of mutations may include exclusively, or in combination: SNP swap libraries, Promoter swap libraries, or any other mutation library described herein.

In one implementation, only a single genetic background is provided. In this case, a collection of distinct genetic backgrounds (microbial mutants) will first be generated from this single background. This may be achieved by applying the primary library of mutations (or some subset thereof) to the given background for example, application of a HTP genetic design library of particular SNPs or a HTP genetic design library of particular promoters to the given genetic background, to create a population (perhaps 100's or 1,000's) of microbial mutants with an identical genetic background except for the particular genetic alteration from the given HTP genetic design library incorporated therein. As detailed below, this embodiment can lead to a combinatorial library or pairwise library.

In another implementation, a collection of distinct known genetic backgrounds may simply be given. As detailed below, this embodiment can lead to a subset of a combinatorial library.

In a particular implementation, the number of genetic backgrounds and genetic diversity between these backgrounds (measured in number of mutations or sequence edit distance or the like) is determined to maximize the effectiveness of this method.

A genetic background may be a natural, native or wild-type strain or a mutated, engineered strain. N distinct background strains may be represented by a vector b. In one example, the background b may represent engineered backgrounds formed by applying N primary mutations m₀=(m₁, m₂, . . . m_{N}) to a wild-type background strain b₀ to form the N mutated background strains b=m₀ b₀=(m₁b₀, m₂b₀, . . . , m_{N} b₀), where mᵢb₀ represents the application of mutation mᵢ to background strain b₀.

In either case (i.e. a single provided genetic background or a collection of genetic backgrounds), the result is a collection of N genetically distinct backgrounds. Relevant phenotypes are measured for each background.

Second, each mutation in a collection of M mutations m₁ is applied to each background within the collection of N background strains b to form a collection of M×N mutants. In the implementation where the N backgrounds were themselves obtained by applying the primary set of mutations m₀ (as described above), the resulting set of mutants will sometimes be referred to as a combinatorial library or a pairwise library. In another implementation, in which a collection of known backgrounds has been provided explicitly, the resulting set of mutants may be referred to as a subset of a combinatorial library. Similar to generation of engineered background vectors, in embodiments, the input interface 202 receives the mutation vector m and the background vector b, and a specified operation such as cross product.

Continuing with the engineered background example above, forming the M×N combinatorial library may be represented by the matrix formed by m₁×m₀ b₀, the cross product of m₁ applied to the N backgrounds of b=m₀ b₀, where each mutation in m₁ is applied to each background strain within b. Each ith row of the resulting M×N matrix represents the application of the ith mutation within m₁ to all the strains within background collection b. In one embodiment, m₁=m₀ and the matrix represents the pairwise application of the same mutations to starting strain b₀. In that case, the matrix is symmetric about its diagonal (M=N), and the diagonal may be ignored in any analysis since it represents the application of the same mutation twice.

In embodiments, forming the M×N matrix may be achieved by inputting into the input interface 202 the compound expression m₁×m₀b₀. The component vectors of the expression may be input directly with their elements explicitly specified, via one or more DNA specifications, or as calls to the library 206 to enable retrieval of the vectors during interpretation by interpreter 204. As described in U.S. patent application Ser. No. 15/140,296, entitled “Microbial Strain Design System and Methods for Improved Large Scale Production of Engineered Nucleotide Sequences,” via the interpreter 204, execution engine 207, order placement engine 208, and factory 210, the LIMS system 200 generates the microbial strains specified by the input expression.

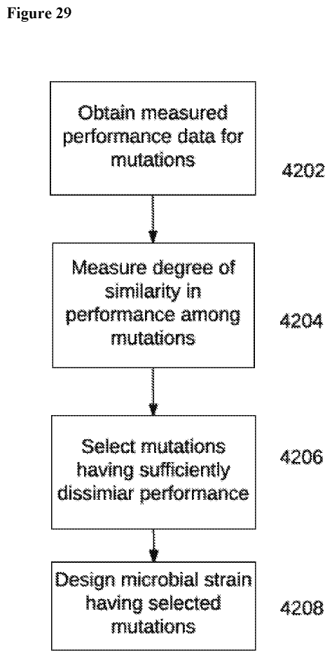

Third, with reference to FIG. 29, the analysis equipment 214 measures phenotypic responses for each mutant within the M×N combinatorial library matrix (4202). As such, the collection of responses can be construed as an M×N Response Matrix R. Each element of R may be represented as rᵢⱼ=y(mᵢ, mⱼ), where y represents the response (performance) of background strain bⱼ within engineered collection b as mutated by mutation mᵢ. For simplicity, and practicality, we assume pairwise mutations where m₁=m₀. Where, as here, the set of mutations represents a pairwise mutation library, the resulting matrix may also be referred to as a gene interaction matrix or, more particularly, as a mutation interaction matrix.

Those skilled in the art will recognize that, in some embodiments, operations related to epistatic effects and predictive strain design may be performed entirely through automated means of the LIMS system 200, e.g., by the analysis equipment 214, or by human implementation, or through a combination of automated and manual means. When an operation is not fully automated, the elements of the LIMS system 200, e.g., analysis equipment 214, may, for example, receive the results of the human performance of the operations rather than generate results through its own operational capabilities. As described elsewhere herein, components of the LIMS system 200, such as the analysis equipment 214, may be implemented wholly or partially by one or more computer systems. In some embodiments, in particular where operations related to predictive strain design are performed by a combination of automated and manual means, the analysis equipment 214 may include not only computer hardware, software or firmware (or a combination thereof), but also equipment operated by a human operator such as that listed in Table 5 below, e.g., the equipment listed under the category of “Evaluate performance.”

Fourth, the analysis equipment 212 normalizes the response matrix. Normalization consists of a manual and/or, in this embodiment, automated processes of adjusting measured response values for the purpose of removing bias and/or isolating the relevant portions of the effect specific to this method. With respect to FIG. 29, the first step 4202 may include obtaining normalized measured data. In general, in the claims directed to predictive strain design and epistasis mapping, the terms “performance measure” or “measured performance” or the like may be used to describe a metric that reflects measured data, whether raw or processed in some manner, e.g., normalized data. In a particular implementation, normalization may be performed by subtracting a previously measured background response from the measured response value. In that implementation, the resulting response elements may be formed as rᵢⱼ=y(mᵢ, mⱼ)−y(mⱼ), where y(mⱼ) is the response of the engineered background strain bⱼ within engineered collection b caused by application of primary mutation mⱼ to parent strain b₀. Note that each row of the normalized response matrix is treated as a response profile for its corresponding mutation. That is, the ith row describes the relative effect of the corresponding mutation mᵢ applied to all the background strains bⱼ for j=1 to N.

With respect to the example of pairwise mutations, the combined performance/response of strains resulting from two mutations may be greater than, less than, or equal to the performance/response of the strain to each of the mutations individually. This effect is known as “epistasis,” and may, in some embodiments, be represented as eᵢⱼ=y(mᵢ, mⱼ)−(y(m)+y(mⱼ)). Variations of this mathematical representation are possible, and may depend upon, for example, how the individual changes biologically interact. As noted above, mutations from the same functional group are more likely to operate by the same mechanism, and are thus more likely to exhibit negative or neutral epistasis on overall host performance. In contrast, mutations from different functional groups are more likely to operate by independent mechanisms, which can lead to improved host performance by reducing redundant mutative effects, for example. Thus, mutations that yield dissimilar responses are more likely to combine in an additive manner than mutations that yield similar responses. This leads to the computation of similarity in the next step.

Fifth, the analysis equipment 214 measures the similarity among the responses—in the pairwise mutation example, the similarity between the effects of the ith mutation and jth (e.g., primary) mutation within the response matrix (4204). Recall that the ith row of R represents the performance effects of the ith mutation mᵢ on the N background strains, each of which may be itself the result of engineered mutations as described above. Thus, the similarity between the effects of the ith and jth mutations may be represented by the similarity sᵢⱼ between the ith and jth rows, ρᵢ and ρⱼ, respectively, to form a similarity matrix S, an example of which is illustrated in FIG. 53. Similarity may be measured using many known techniques, such as cross-correlation or absolute cosine similarity, e.g., sᵢⱼ=abs(cos(ρᵢ, ρⱼ)).

As an alternative or supplement to a metric like cosine similarity, response profiles may be clustered to determine degree of similarity. Clustering may be performed by use of a distance-based clustering algorithms (e.g. k-mean, hierarchical agglomerative, etc.) in conjunction with suitable distance measure (e.g. Euclidean, Hamming, etc.). Alternatively, clustering may be performed using similarity based clustering algorithms (e.g. spectral, min-cut, etc.) with a suitable similarity measure (e.g. cosine, correlation, etc.). Of course, distance measures may be mapped to similarity measures and vice-versa via any number of standard functional operations (e.g., the exponential function). In one implementation, hierarchical agglomerative clustering may be used in conjunction absolute cosine similarity. (See FIG. 54A).

As an example of clustering, let C be a clustering of mutations mᵢ into k distinct clusters. Let C be the cluster membership matrix, where cᵢⱼ is the degree to which mutation i belongs to cluster j, a value between 0 and 1. The cluster-based similarity between mutations i and j is then given by Cᵢ×Cⱼ (the dot product of the ith and jth rows of C). In general, the cluster-based similarity matrix is given by CC^{T} (that is, C times C-transpose). In the case of hard-clustering (a mutation belongs to exactly one cluster), the similarity between two mutations is 1 if they belong to the same cluster and 0 if not.

As is described in Costanzo, The Genetic Landscape of a Cell, Science, Vol. 327, Issue 5964, Jan. 22, 2010, pp. 425-431 (incorporated by reference herein in its entirety), such a clustering of mutation response profiles relates to an approximate mapping of a cell's underlying functional organization. That is, mutations that cluster together tend to be related by an underlying biological process or metabolic pathway. Such mutations are referred to herein as a “functional group.” The key observation of this method is that if two mutations operate by the same biological process or pathway, then observed effects (and notably observed benefits) may be redundant. Conversely, if two mutations operate by distant mechanism, then it is less likely that beneficial effects will be redundant.

Sixth, based on the epistatic effect, the analysis equipment 214 selects pairs of mutations that lead to dissimilar responses, e.g., their cosine similarity metric falls below a similarity threshold, or their responses fall within sufficiently separated clusters, (e.g., in FIG. 53 and FIG. 54A) as shown in FIG. 29 (4206). Based on their dissimilarity, the selected pairs of mutations should consolidate into background strains better than similar pairs.

Based upon the selected pairs of mutations that lead to sufficiently dissimilar responses, the LIMS system (e.g., all of or some combination of interpreter 204, execution engine 207, order placer 208, and factory 210) may be used to design microbial strains having those selected mutations (4208). In embodiments, as described below and elsewhere herein, epistatic effects may be built into, or used in conjunction with the predictive model to weight or filter strain selection.

It is assumed that it is possible to estimate the performance (a.k.a. score) of a hypothetical strain obtained by consolidating a collection of mutations from the library into a particular background via some preferred predictive model. A representative predictive model utilized in the taught methods is provided in the below section entitled “Predictive Strain Design” that is found in the larger section of: “Computational Analysis and Prediction of Effects of Genome-Wide Genetic Design Criteria.”

When employing a predictive strain design technique such as linear regression, the analysis equipment 214 may restrict the model to mutations having low similarity measures by, e.g., filtering the regression results to keep only sufficiently dissimilar mutations. Alternatively, the predictive model may be weighted with the similarity matrix. For example, some embodiments may employ a weighted least squares regression using the similarity matrix to characterize the interdependencies of the proposed mutations. As an example, weighting may be performed by applying the “kernel” trick to the regression model. (To the extent that the “kernel trick” is general to many machine learning modeling approaches, this re-weighting strategy is not restricted to linear regression.)

Such methods are known to one skilled in the art. In embodiments, the kernel is a matrix having elements 1−w*sᵢⱼ where 1 is an element of the identity matrix, and w is a real value between 0 and 1. When w=0, this reduces to a standard regression model. In practice, the value of w will be tied to the accuracy (r² value or root mean square error (RMSE)) of the predictive model when evaluated against the pairwise combinatorial constructs and their associate effects y(mᵢ, mⱼ). In one simple implementation, w is defined as w=1−r². In this case, when the model is fully predictive, w=1−r²=0 and consolidation is based solely on the predictive model and epistatic mapping procedure plays no role. On the other hand, when the predictive model is not predictive at all, w=1−r²=1 and consolidation is based solely on the epistatic mapping procedure. During each iteration, the accuracy can be assessed to determine whether model performance is improving.

It should be clear that the epistatic mapping procedure described herein does not depend on which model is used by the analysis equipment 214. Given such a predictive model, it is possible to score and rank all hypothetical strains accessible to the mutation library via combinatorial consolidation.

In some embodiments, to account for epistatic effects, the dissimilar mutation response profiles may be used by the analysis equipment 214 to augment the score and rank associated with each hypothetical strain from the predictive model. This procedure may be thought of broadly as a re-weighting of scores, so as to favor candidate strains with dissimilar response profiles (e.g., strains drawn from a diversity of clusters). In one simple implementation, a strain may have its score reduced by the number of constituent mutations that do not satisfy the dissimilarity threshold or that are drawn from the same cluster (with suitable weighting). In a particular implementation, a hypothetical strain's performance estimate may be reduced by the sum of terms in the similarity matrix associated with all pairs of constituent mutations associated with the hypothetical strain (again with suitable weighting) Hypothetical strains may be re-ranked using these augmented scores. In practice, such re-weighting calculations may be performed in conjunction with the initial scoring estimation.

The result is a collection of hypothetical strains with score and rank augmented to more effectively avoid confounding epistatic interactions. Hypothetical strains may be constructed at this time, or they may be passed to another computational method for subsequent analysis or use.

Those skilled in the art will recognize that epistasis mapping and iterative predictive strain design as described herein are not limited to employing only pairwise mutations, but may be expanded to the simultaneous application of many more mutations to a background strain. In another embodiment, additional mutations may be applied sequentially to strains that have already been mutated using mutations selected according to the predictive methods described herein. In another embodiment, epistatic effects are imputed by applying the same genetic mutation to a number of strain backgrounds that differ slightly from each other, and noting any significant differences in positive response profiles among the modified strain backgrounds.

### HTP Conjugating Conjugation to Introduce Exogenous DNA

The present disclosure also provides methods for transferring genetic material from donor microorganism cells to recipient cells of a Saccharopolyspora microorganism. The donor microorganism cells can be any suitable donor cells, including but not limited to E. coli cells. The recipient microorganism cells can be a Saccharopolyspora species, such as a S. spinosa strain.

In general, the methods comprise the following steps of: (1) subculturing recipient cells to mid-exponential phase (optional); (2) subculturing donor cells to mid-exponential phase (optional); (3) combining donor and recipient cells; (4) plating donor and recipient cell mixture on conjugation media; (5) incubating plates to allow cells to conjugate; (6) applying antibiotic selection against donor cells; (7) Applying antibiotic selection against non-integrated recipient cells; and (8) further incubating plates to allow for the outgrowth of integrated recipient cells.

Inventors of the present application discovered conditions that can be optimized which lead to surprisingly increased frequency of exogenous DNA conjugation in S. spinosa. Such conditions include, but not limited to (1) recipient cells are washed (e.g., before conjugating); (2) donor cells and recipient cells are conjugated at a relatively lower temperature; (3) recipient cells are sub-cultured for an extended period of time before conjugating; (4) a proper ratio of donor cells:recipient cells for conjugation; (5) a proper timing of delivering an antibiotic drug for selection against the donor cells to the conjugation mixture; (6) a proper timing of an antibiotic drug for selection against the recipient cells to the conjugation mixture; (7) a proper timing of drying the conjugation media plated with donor and recipient cell mixture; (8) a high concentration of glucose; (9) a proper concentration of donor cells; and (10) a proper concentration of recipient.

In some embodiments, at least two, three, four, five, six, seven or more of the following conditions are utilized which lead to increased conjugation:

(1) recipient cells are washed;
(2) donor cells and recipient cells are conjugated at a temperature of about 25° C., 26° C., 27° C., 28° C., 29° C., 30° C., 31°, 32° C., 33° C., such as at 30° C.;
(3) recipient cells are sub-cultured for at least about 40, 41, 42, 43, 44, 45, 46, 47, 48, 49, 50, 51, 52, 53, 54, 55 hours before conjugating, such as for about 48 hours;
(4) the ratio of donor cells:recipient cells for conjugation is about 1:0.5, 1:0.6, 1:0.7, 1:08, 1:0.9, 1:1.0, 1:1.1, 1:1.2, 1:1.3, 1:1.4, 1:1.5, 1:1.6, 1:1.7, 1:1.8 1:1.9 or 1:2.0, such as from about 1:0.6 to 1:1.0;
(5) an antibiotic drug for selection against the donor cells is delivered to the mixture about 15, 16, 17, 18, 19, 20, 21, 22, 23, 24, 25, 26, 27, 28, 29, or 30 hours after the donor cells and the recipient cells are mixed, such as about 24 hours after.
(6) an antibiotic drug for selection against the recipient cells is delivered to the mixture about 35, 36, 37, 38, 39, 40, 41, 42, 43, 44, 45, 46, 47, 48, 49, 50, 51, 52, 53, 54, or 55 hours, such as from about 40 to 48 hours after the donor cells and the recipient cells are mixed;
(7) the conjugation media plated with donor and recipient cell mixture is dried for at least about 1 hour, 2 hours, 3 hours, 4 hours, 5 hours, 6 hours, 7 hours, 8 hours, 9 hours, 10 hours, 11 hours, 12 hours, 13 hours, 14 hours or 15 hours;
(8) the conjugation media comprises at least about 0.5 g/L, 1 g/L, 1.5 g/L, 2 g/L, 2.5 g/L, 3 g/L, 3.5 g/L, 4 g/L, 4.5 g/L, 5 g/L, 5.5 g/L, 6 g/L, 6.5 g/L, 7 g/L, 7.5 g/L, 8 g/L, 8.5 g/L, 9 g/L, 9.5 g/L, 10 g/L, or more glucose;
(9) the concentration of donor cells is about OD600=0.1, 0.15, 0.2, 0.25, 0.30, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.65, 0.70, 0.75, 0.80, 0.85, 0.90, 0.95, 1.0; and
(10) the concentration of recipient cells is about OD540=1.0, 2.0, 3.0, 4.0, 5.0, 6.0, 7.0, 8.0, 9.0, 10.0, 10.5, 11.0, 11.5, 12.0, 12.5, 13.0, 13.5, 14.0, 14.5, or 15.0.

In some embodiments, the total number of donor cells or recipient cells in the mixture is about 5×10⁶, 6×10⁶, 7×10⁶, 8×10⁶, or about 9×10⁶.

In some embodiments, the donor cells are E. coli cells, and the antibiotic drug for selection against the donor cells is nalidixic. In some embodiments, the concentration of nalidixic is about 50, 60, 70, 80, 90, 100, 110, 120, 130, 140, 150, 60, 170, 180, 190, or 200 μg/ml.

In some embodiments, the antibiotic drug for selection against the recipient cells is apramycin, and the concentration is about 50, 60, 70, 80, 90, 100, 110, 120, 130, 140, 150, 60, 170, 180, 190, 200, 210, 220, 230, 240, 250, 260, 270, 280, 290, 300 μg/ml.

The methods as described herein can be performed in a high-throughput process. In some embodiments, the methods are performed on a 48-well Q-trays. In some embodiments, the high-throughput process is partially or fully automated.

In some embodiments, the mixture of donor cells and recipient cells is a liquid mixture, and ample volume of the liquid mixture is plated on the medium with a rocking motion, wherein the liquid mixture is dispersed over the whole area of the medium.

In some embodiments, the method comprises automated process of transferring exconjugants by colony picking with yeast pins for subsequent inoculation of recipient cells with integrated DNA provided by the donor cells. In some embodiments, the colony picking is performed in either a dipping motion, or a stirring motion.

In some embodiments, the method is performed with at least two, three, four, five, six, or seven of the following conditions: (1) recipient cells are washed before conjugating; (2) donor cells and recipient cells are conjugated at a temperature of about 30° C.; (3) recipient cells are sub-cultured for at least about 48 hours before conjugating; (4) the ratio of donor cells:recipient cells for conjugation is about 1:0.8; (5) an antibiotic drug for selection against the donor cells is delivered to the mixture about 20 hours after the donor cells and the recipient cells are mixed; (6) the amount of the donor cells or the amount of the recipient cells in the mixture is about 7×106, and (7) the conjugation media comprises about 6 g/L glucose.

### Pathway Refactoring

The present disclosure provides methods for pathway refactoring. As used herein, the term “pathway refactoring” refers to the process of constructing one or more fully or a partially optimal biosynthetic pathway in a microorganism. In some embodiments, the biosynthetic pathway is associated with synthesis of one or more products of interest, such as spinosyns.

The methods of pathway refactoring can utilize one or more tools of the present disclosure. Without wishing to be bound by any particular theory, the methods of pathway refactoring can fine-tune the activity of one or more genes directly involved in the biosynthetic pathway, or the activity of one or more genes indirectly involved in the biosynthetic pathway (e.g., genes that can indirectly affect the biosynthesis of a given product of interest. In some embodiments, to fine-tune one or more genes involved in the biosynthetic pathway, the methods comprise utilizing one or more genetic diversity libraries of the present disclosure, including but not limited to a promoter ladder library, a RB S ladder library, a terminator library, a stop/start codon library, etc. In some embodiments, the activity of one or more genes involved in the biosynthetic pathway is modified by at least one genetic tool as disclosed herein. In some embodiments, strains bearing modified genes can be screened through the high through put system as described in the present disclosure to identify strains having improved performance compared to a check strain, such as a strain without the modification.

As a result, one, two, three, four, five, six, seven, eight, nine, ten or more genes involved in the biosynthetic pathway are fine-tuned. In some embodiments, any number of genes are fine-tuned. In some embodiments, the fine-tuned genes are in the same signaling pathway or synthetic pathway. In some embodiments, the fine-tuned genes are in different signaling pathways or synthetic pathways. In some embodiments, activity of certain genes is modified as necessary, as long as the modification results in improved performance of the strain. In some embodiments, the activity of one or more genes are up-regulated compared to that in a check strain. In some embodiments, the activity of one or more genes are down-regulated compared to that in a check strain. In some embodiments, the timing of expression of one or more genes is changed compared to that in a check strain. In some embodiments, the location of expression of one or more genes is changed compared to that in a check strain. In some embodiments, the activity of one or more genes involved in the rate determining step (RD S) or rate-limiting step is modified compared to that in a check strain. In some embodiments, one, two, three, four, five, six, seven, eight, nine, ten or more modified gene locus are consolidated to create strains with further fine-tuned biosynthetic pathway.

In some embodiments, the methods of pathway refactoring comprise incorporating genetic material into the genome of a microorganism of the present disclosure. In some embodiments, the microorganism is Saccharopolyspora sp., such as Saccharopolyspora spinosa, and the genetic material is incorporated into a specific position (e.g., a “landing pad”) in the genome of the microorganism. In some embodiments, the specific position is selected from the neutral integration sites (NISs) of the present disclosure as described herein.

In some embodiments, the genetic material is introduced into a microorganism of the present disclosure via a self-replicable vector. In some embodiments, the microorganism is Saccharopolyspora sp., such as Saccharopolyspora spinosa, and the genetic material is introduced into the microorganism through a self-replicating plasmid of the present disclosure as described herein.

### Organisms Amenable to Genetic Design

The disclosed HTP genomic engineering platform is exemplified with industrial microbial cell cultures (e.g., Saccharopolyspora spp.), but is applicable to any host cell organism where desired traits can be identified in a population of genetic mutants.

Thus, as used herein, the term “microorganism” should be taken broadly. It includes, but is not limited to, the two prokaryotic domains, Bacteria and Archaea, as well as certain eukaryotic fungi and protists. However, in certain aspects, “higher” eukaryotic organisms such as insects, plants, and animals can be utilized in the methods taught herein.

Suitable host cells include, but are not limited to: Saccharopolyspora antimicrobia, Saccharopolyspora cavernae, Saccharopolyspora cebuensis, Saccharopolyspora dendranthemae, Saccharopolyspora erythraea, Saccharopolyspora flava, Saccharopolyspora ghardaiensis, Saccharopolyspora gloriosae, Saccharopolyspora gregorii, Saccharopolyspora halophile, Saccharopolyspora halotolerans, Saccharopolyspora hirsute, Saccharopolyspora horde, Saccharopolyspora indica, Saccharopolyspora jiangxiensis, Saccharopolyspora lacisalsi, Saccharopolyspora phatthalungensis, Saccharopolyspora qijiaojingensis, Saccharopolyspora rectivirgula, Saccharopolyspora rosea, Saccharopolyspora shandongensis, Saccharopolyspora spinosa, Saccharopolyspora spinosporotrichia, Saccharopolyspora taberi, Saccharopolyspora thermophile, and Saccharopolyspora tripterygii.

In some embodiments, the host cells are selected from Saccharopolyspora indianesis (ATCC® BAA-2551™), Saccharopolyspora erythraea (Waksman) Labeda (ATCC® 31772™), Saccharopolyspora erythraea (Waksman) Labeda (ATCC® 11912™), Saccharopolyspora rectivirgula (Krasil'nikov and Agre) Korn-Wendisch et al. (ATCC® 29034™), Saccharopolyspora hirsuta subsp. hirsuta Lacey and Goodfellow (ATCC® 27875™), NEB#998 (ATCC® 98102™), Saccharopolyspora hirsuta subsp. kobensis (Iwasaki et al.) Lacey (ATCC® 20501™), Saccharopolyspora rectivirgula (Krasil'nikov and Agre) Korn-Wendisch et al. (ATCC® 29035™), Saccharopolyspora erythraea (Waksman) Labeda (ATCC® 11635D-5™) ATCC® Number: 11635D-5™, Saccharopolyspora taberi (Labeda) Korn-Wendisch et al. (ATCC® 49842™), Saccharopolyspora hirsuta subsp. hirsuta Lacey and Goodfellow (ATCC® 27876™), Saccharopolyspora aurantiaca Etienne et al. (ATCC® 51351™), Saccharopolyspora gregorii Goodfellow et al. (ATCC® 51265™), Saccharopolyspora erythraea (Waksman) Labeda (ATCC® 11635™), Saccharopolyspora rectivirgula (Krasil'nikov and Agre) Korn-Wendisch et al. (ATCC® 33515™), Saccharopolyspora rectivirgula (Krasil'nikov and Agre) Korn-Wendisch et al. (ATCC® 15347™), Saccharopolyspora spinosa Mertz and Yao (ATCC® 49460™), Saccharopolyspora rectivirgula (Krasil'nikov and Agre) Korn-Wendisch et al. (ATCC® 21450™), Saccharopolyspora hordei Goodfellow et al. (ATCC® 49856™), Saccharopolyspora rectivirgula (Krasil'nikov and Agre) Korn-Wendisch et al. (ATCC® 29681™), pIJ43 [MCB1023] (ATCC® 39156™), pOJ31 (ATCC® 77416™), and Saccharopolyspora rectivirgula (21451).

### Generating Genetic Diversity Pools for Utilization in the Genetic Design & HTP Microbial Engineering Platform

In some embodiments, the methods of the present disclosure are characterized as genetic design. As used herein, the term genetic design refers to the reconstruction or alteration of a host organism's genome through the identification and selection of the most optimum variants of a particular gene, portion of a gene, promoter, stop codon, 5′UTR, 3′UTR, ribosomal binding site, terminator, or other DNA sequence to design and create new superior host cells.

In some embodiments, a first step in the genetic design methods of the present disclosure is to obtain an initial genetic diversity pool population with a plurality of sequence variations from which a new host genome may be reconstructed.

In some embodiments, a subsequent step in the genetic design methods taught herein is to use one or more of the aforementioned HTP molecular tool sets (e.g. SNP swapping or promoter swapping) to construct HTP genetic design libraries, which then function as drivers of the genomic engineering process, by providing libraries of particular genomic alterations for testing in a host cell.

Harnessing Diversity Pools from Existing Wild-Type Strains

In some embodiments, the present disclosure teaches methods for identifying the sequence diversity present among microbes of a given wild-type population. Therefore, a diversity pool can be a given number n of wild-type microbes utilized for analysis, with said microbes' genomes representing the “diversity pool.”

In some embodiments, the diversity pools can be the result of existing diversity present in the natural genetic variation among said wild-type microbes. This variation may result from strain variants of a given host cell or may be the result of the microbes being different species entirely. Genetic variations can include any differences in the genetic sequence of the strains, whether naturally occurring or not. In some embodiments, genetic variations can include SNPs swaps, PRO swaps, Start/Stop Codon swaps, STOP swaps, transposon mutagenesis diversity libraries, ribosomal binding site diversity libraries, anti-metabolite selection/fermentation product resistance libraries, among others.

Harnessing Diversity Pools from Existing Industrial Strain Variants

In other embodiments of the present disclosure, diversity pools are strain variants created during traditional strain improvement processes (e.g., one or more host organism strains generated via random mutation and selected for improved yields over the years). Thus, in some embodiments, the diversity pool or host organisms can comprise a collection of historical production strains.

In particular aspects, a diversity pool may be an original parent microbial strain (S₁) with a “baseline” genetic sequence at a particular time point (S₁Gen₁) and then any number of subsequent offspring strains (S₂, S₃, S₄, S₅, etc., generalizable to S₂₋ₙ) that were derived/developed from said S₁ strain and that have a different genome (S₂₋ₙGen₂₋ₙ), in relation to the baseline genome of S₁.

For example, in some embodiments, the present disclosure teaches sequencing the microbial genomes in a diversity pool to identify the SNP's present in each strain. In one embodiment, the strains of the diversity pool are historical microbial production strains. Thus, a diversity pool of the present disclosure can include for example, an industrial base strain, and one or more mutated industrial strains produced via traditional strain improvement programs.

Once all SNPs in the diversity pool are identified, the present disclosure teaches methods of SNP swapping and screening methods to delineate (i.e. quantify and characterize) the effects (e.g. creation of a phenotype of interest) of SNPs individually and in groups. Thus, as aforementioned, an initial step in the taught platform can be to obtain an initial genetic diversity pool population with a plurality of sequence variations, e.g. SNPs. Then, a subsequent step in the taught platform can be to use one or more of the aforementioned HTP molecular tool sets (e.g. SNP swapping) to construct HTP genetic design libraries, which then function as drivers of the genomic engineering process, by providing libraries of particular genomic alterations for testing in a microbe.

In some embodiments, the SNP swapping methods of the present disclosure comprise the step of introducing one or more SNPs identified in a mutated strain (e.g., a strain from amongst S₂₋ₙGen₂₋ₙ) to a base strain (S₁Gen₁) or wild-type strain.

In other embodiments, the SNP swapping methods of the present disclosure comprise the step of removing one or more SNPs identified in a mutated strain (e.g., a strain from amongst S₂₋ₙGen₂₋ₙ).

Creating Diversity Pools Via Mutagenesis

In some embodiments, the mutations of interest in a given diversity pool population of cells can be artificially generated by any means for mutating strains, including mutagenic chemicals, or radiation. The term “mutagenizing” is used herein to refer to a method for inducing one or more genetic modifications in cellular nucleic acid material.

The term “genetic modification” refers to any alteration of DNA. Representative gene modifications include nucleotide insertions, deletions, substitutions, and combinations thereof, and can be as small as a single base or as large as tens of thousands of bases. Thus, the term “genetic modification” encompasses inversions of a nucleotide sequence and other chromosomal rearrangements, whereby the position or orientation of DNA comprising a region of a chromosome is altered. A chromosomal rearrangement can comprise an intrachromosomal rearrangement or an interchromosomal rearrangement.

In one embodiment, the mutagenizing methods employed in the presently claimed subject matter are substantially random such that a genetic modification can occur at any available nucleotide position within the nucleic acid material to be mutagenized. Stated another way, in one embodiment, the mutagenizing does not show a preference or increased frequency of occurrence at particular nucleotide sequences.

The methods of the disclosure can employ any mutagenic agent including, but not limited to: ultraviolet light, X-ray radiation, gamma radiation, N-ethyl-N-nitrosourea (ENU), methyinitrosourea (MNU), procarbazine (PRC), triethylene melamine (TEM), acrylamide monomer (AA), chlorambucil (CHL), melphalan (MLP), cyclophosphamide (CPP), diethyl sulfate (DES), ethyl methane sulfonate (EMS), methyl methane sulfonate (MMS), 6-mercaptopurine (6-MP), mitomycin-C (MMC), N-methyl-N′-nitro-N-nitrosoguanidine (MNNG), ³H₂O, and urethane (UR) (See e.g., Rinchik, 1991; Marker et al., 1997; and Russell, 1990). Additional mutagenic agents are well known to persons having skill in the art, including those described in iephb.nwsu/˜spirov/hazard/mutagen_1st.

In some embodiments, one or more mutagenesis strategies described in the present disclosure can be employed to generate, screen, and consolidate mutations of interest. In some embodiments, genetic tools described in the present disclosure can be used to create genetic diversity. For example, the promoter swap method, the SNP swap method, the start/stop codon swap method, the terminator swap method, the transposon mutagenesis method, the ribosomal binding site method, the anti-metabolite selection/fermentation product resistance method, or any combination thereof, can be utilized as other opportunities to create genetic diversity.

The term “mutagenizing” also encompasses a method for altering (e.g., by targeted mutation) or modulating a cell function, to thereby enhance a rate, quality, or extent of mutagenesis. For example, a cell can be altered or modulated to thereby be dysfunctional or deficient in DNA repair, mutagen metabolism, mutagen sensitivity, genomic stability, or combinations thereof. Thus, disruption of gene functions that normally maintain genomic stability can be used to enhance mutagenesis. Representative targets of disruption include, but are not limited to DNA ligase I (Bentley et al., 2002) and casein kinase I (U.S. Pat. No. 6,060,296).

In some embodiments, site-specific mutagenesis (e.g., primer-directed mutagenesis using a commercially available kit such as the Transformer Site Directed mutagenesis kit (Clontech)) is used to make a plurality of changes throughout a nucleic acid sequence in order to generate nucleic acid encoding a cleavage enzyme of the present disclosure.

The frequency of genetic modification upon exposure to one or more mutagenic agents can be modulated by varying dose and/or repetition of treatment, and can be tailored for a particular application.

Thus, in some embodiments, “mutagenesis” as used herein comprises all techniques known in the art for inducing mutations, including error-prone PCR mutagenesis, oligonucleotide-directed mutagenesis, site-directed mutagenesis, transposon mutagenesis, and iterative sequence recombination by any of the techniques described herein.

Single Locus Mutations to Generate Diversity

In some embodiments, the present disclosure teaches mutating cell populations by introducing, deleting, or replacing selected portions of genomic DNA. Thus, in some embodiments, the present disclosure teaches methods for targeting mutations to a specific locus. In other embodiments, the present disclosure teaches the use of gene editing technologies such as ZFNs, TALENS, or CRISPR, to selectively edit target DNA regions.

In other embodiments, the present disclosure teaches mutating selected DNA regions outside of the host organism, and then inserting the mutated sequence back into the host organism. For example, in some embodiments, the present disclosure teaches mutating native or synthetic promoters to produce a range of promoter variants with various expression properties (see promoter ladder infra). In other embodiments, the present disclosure is compatible with single gene optimization techniques, such as ProSAR (Fox et al. 2007. “Improving catalytic function by ProSAR-driven enzyme evolution.” Nature Biotechnology Vol 25 (3) 338-343, incorporated by reference herein).

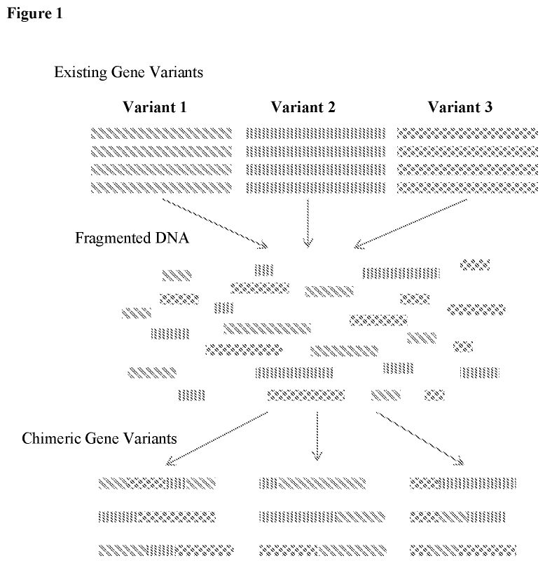

In some embodiments, the selected regions of DNA are produced in vitro via gene shuffling of natural variants, or shuffling with synthetic oligos, plasmid-plasmid recombination, virus plasmid recombination, virus-virus recombination. In other embodiments, the genomic regions are produced via error-prone PCR (see e.g., FIG. 1).

In some embodiments, generating mutations in selected genetic regions is accomplished by “reassembly PCR.” Briefly, oligonucleotide primers (oligos) are synthesized for PCR amplification of segments of a nucleic acid sequence of interest, such that the sequences of the oligonucleotides overlap the junctions of two segments. The overlap region is typically about 10 to 100 nucleotides in length. Each of the segments is amplified with a set of such primers. The PCR products are then “reassembled” according to assembly protocols. In brief, in an assembly protocol, the PCR products are first purified away from the primers, by, for example, gel electrophoresis or size exclusion chromatography. Purified products are mixed together and subjected to about 1-10 cycles of denaturing, reannealing, and extension in the presence of polymerase and deoxynucleoside triphosphates (dNTP's) and appropriate buffer salts in the absence of additional primers (“self-priming”). Subsequent PCR with primers flanking the gene are used to amplify the yield of the fully reassembled and shuffled genes.

In some embodiments of the disclosure, mutated DNA regions, such as those discussed above, are enriched for mutant sequences so that the multiple mutant spectrum, i.e. possible combinations of mutations, is more efficiently sampled. In some embodiments, mutated sequences are identified via a mutS protein affinity matrix (Wagner et al., Nucleic Acids Res. 23(19):3944-3948 (1995); Su et al., Proc. Natl. Acad. Sci. (U.S.A.), 83:5057-5061 (1986)) with a preferred step of amplifying the affinity-purified material in vitro prior to an assembly reaction. This amplified material is then put into an assembly or reassembly PCR reaction as described in later portions of this application.

Promoter Ladders

Promoters regulate the rate at which genes are transcribed and can influence transcription in a variety of ways. Constitutive promoters, for example, direct the transcription of their associated genes at a constant rate regardless of the internal or external cellular conditions, while regulatable promoters increase or decrease the rate at which a gene is transcribed depending on the internal and/or the external cellular conditions, e.g. growth rate, temperature, responses to specific environmental chemicals, and the like. Promoters can be isolated from their normal cellular contexts and engineered to regulate the expression of virtually any gene, enabling the effective modification of cellular growth, product yield and/or other phenotypes of interest.

In some embodiments, the present disclosure teaches methods for producing promoter ladder libraries for use in downstream genetic design methods. For example, in some embodiments, the present disclosure teaches methods of identifying one or more promoters and/or generating variants of one or more promoters within a host cell, which exhibit a range of expression strengths, or superior regulatory properties. A particular combination of these identified and/or generated promoters can be grouped together as a promoter ladder, which is explained in more detail below.

In some embodiments, the present disclosure teaches the use of promoter ladders. In some embodiments, the promoter ladders of the present disclosure comprise promoters exhibiting a continuous range of expression profiles. For example, in some embodiments, promoter ladders are created by: identifying natural, native, or wild-type promoters that exhibit a range of expression strengths in response to a stimuli, or through constitutive expression (see e.g., FIG. 13 and FIGS. 21-23). These identified promoters can be grouped together as a promoter ladder.

In some embodiments, promoter ladders comprise at least two promoters with different expression profiles. In some embodiments, promoter ladders comprise at least three promoters with different expression profiles. In some embodiments, promoter ladders comprise at least four promoters with different expression profiles. In some embodiments, promoter ladders comprise at least five promoters with different expression profiles. In some embodiments, promoter ladders comprise at least six promoters with different expression profiles. In some embodiments, promoter ladders comprise at least seven promoters with different expression profiles.

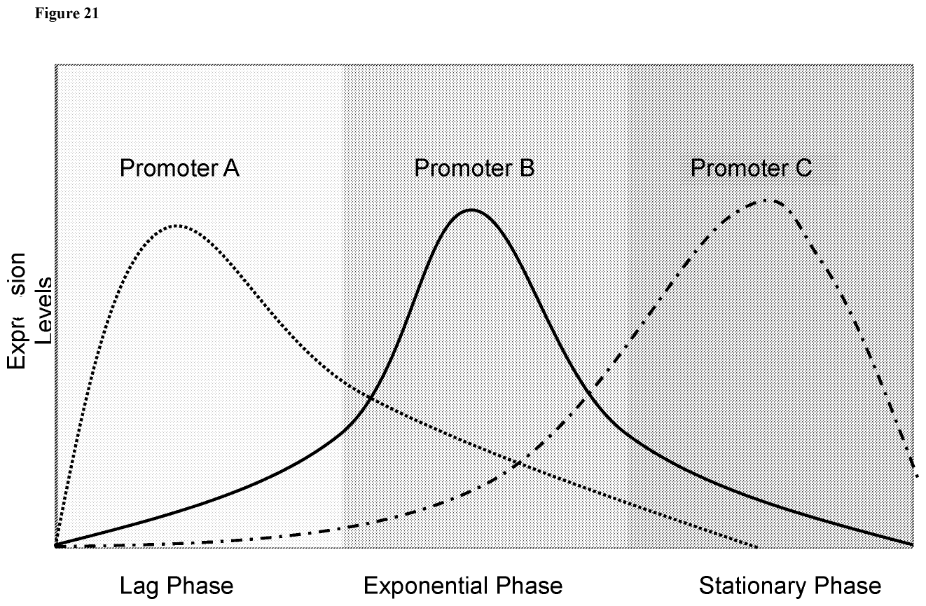

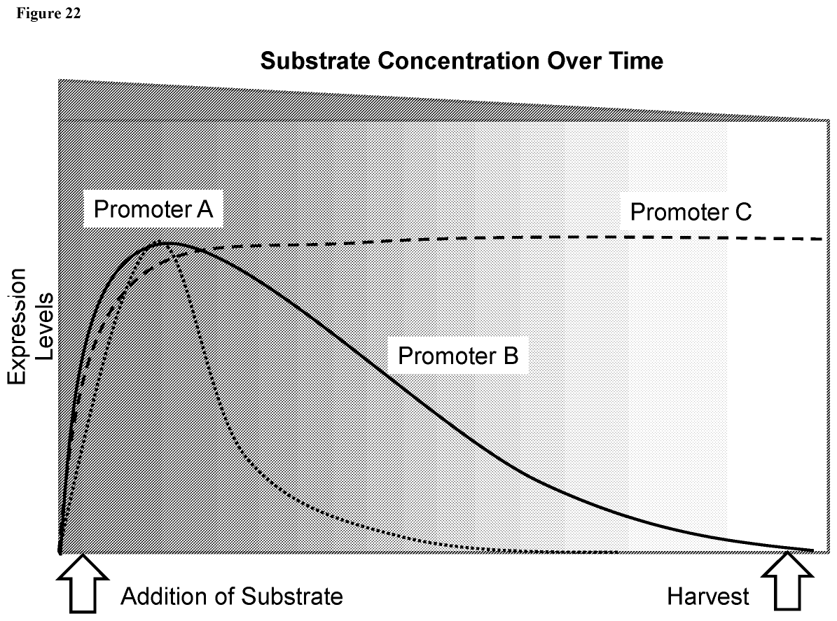

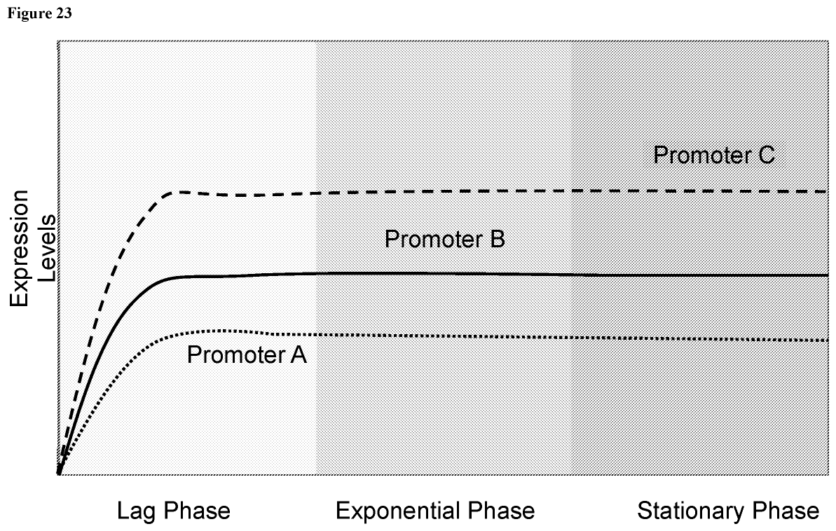

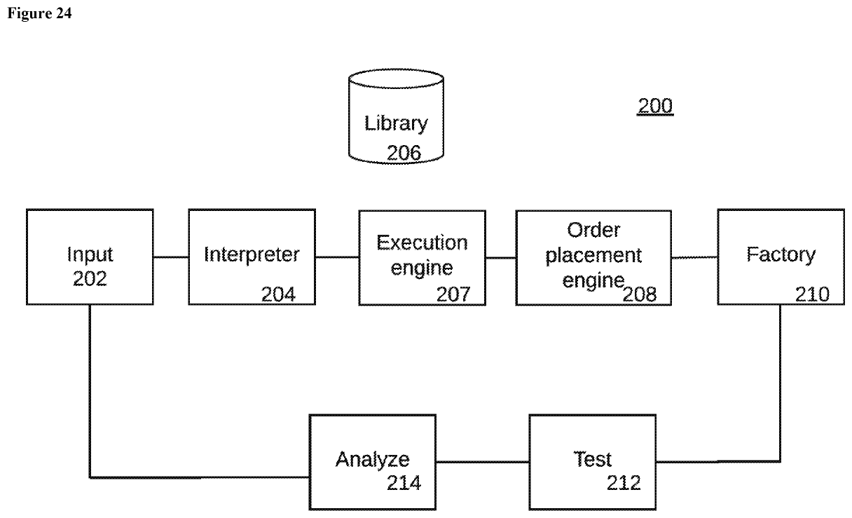

In other embodiments, the present disclosure teaches the creation of promoter ladders exhibiting a range of expression profiles across different conditions. For example, in some embodiments, the present disclosure teaches creating a ladder of promoters with expression peaks spread throughout the different stages of a fermentation (see e.g., FIG. 21). In other embodiments, the present disclosure teaches creating a ladder of promoters with different expression peak dynamics in response to a specific stimulus (see e.g., FIG. 22). Persons skilled in the art will recognize that the regulatory promoter ladders of the present disclosure can be representative of any one or more regulatory profiles.

In some embodiments, the promoter ladders of the present disclosure are designed to perturb gene expression in a predictable manner across a continuous range of responses. In some embodiments, the continuous nature of a promoter ladder confers strain improvement programs with additional predictive power. For example, in some embodiments, swapping promoters or termination sequences of a selected metabolic pathway can produce a host cell performance curve, which identifies the most optimum expression ratio or profile; producing a strain in which the targeted gene is no longer a limiting factor for a particular reaction or genetic cascade, while also avoiding unnecessary over expression or misexpression under inappropriate circumstances. In some embodiments, promoter ladders are created by: identifying natural, native, or wild-type promoters exhibiting the desired profiles. In other embodiments, the promoter ladders are created by mutating naturally occurring promoters to derive multiple mutated promoter sequences. Each of these mutated promoters is tested for effect on target gene expression. In some embodiments, the edited promoters are tested for expression activity across a variety of conditions, such that each promoter variant's activity is documented/characterized/annotated and stored in a database. The resulting edited promoter variants are subsequently organized into promoter ladders arranged based on the strength of their expression (e.g., with highly expressing variants near the top, and attenuated expression near the bottom, therefore leading to the term “ladder”).

In some embodiments, the present disclosure teaches promoter ladders that are a combination of identified naturally occurring promoters and mutated variant promoters.

In some embodiments, the present disclosure teaches methods of identifying natural, native, or wild-type promoters that satisfied both of the following criteria: 1) represented a ladder of constitutive promoters; and 2) could be encoded by short DNA sequences, ideally less than 100 base pairs. In some embodiments, constitutive promoters of the present disclosure exhibit constant gene expression across two selected growth conditions (typically compared among conditions experienced during industrial cultivation). In some embodiments, the promoters of the present disclosure will consist of a ˜20, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, or more base pairs core promoter. In some embodiments, there is a 5′ UTR. In some embodiments, the 5′UTR is between about 5, 10, 15, 20, 25, 30, 35, 40, 45, 50, 55, 60, 65, 70, 75, 80, 85, 90, 95, 100, or more base pairs in length.

In some embodiments, one or more of the aforementioned identified naturally occurring promoter sequences are chosen for gene editing. In some embodiments, the natural promoters are edited via any of the mutation methods described supra. In other embodiments, the promoters of the present disclosure are edited by synthesizing new promoter variants with the desired sequence.

The entire disclosure of U.S. Patent Application No. 62/264,232, filed on Dec. 7, 2015 and PCT WO 2017/100376, filed on Dec. 7, 2016, each of which is hereby incorporated by reference in its entirety for all purposes.

A non-exhaustive list of the promoters of the present disclosure is provided in the below Table 1.

TABLE 1
Selected promoter sequences of the present disclosure.

| SEQ ID No. | Promoter Name |
|---|---|
| 1 | P7160 |
| 2 | P7253 |
| 3 | P6681 |
| 4 | P6316 |
| 5 | P6806 |
| 6 | P3159 |
| 7 | P0757 |
| 8 | P5011 |
| 9 | P1409 |
| 10 | P4735 |
| 11 | P2900 |
| 12 | P0801 |
| 13 | P21 |
| 14 | PA9 |
| 15 | PA3 |
| 16 | PB4 |
| 17 | PB12 |
| 18 | PB1 |
| 19 | PC1 |
| 20 | P72 |
| 21 | P-C4-1 |
| 22 | P-A5-19 |
| 23 | P-C4-14 |
| 24 | P-D1-7 |
| 25 | P1 |
| 26 | P2 |
| 27 | P3 |
| 28 | P3v2 |
| 29 | P4 |
| 30 | P4v2 |
| 31 | P5 |
| 32 | P5v2 |
| 33 | P6 |
| 34 | P7 |
| 35 | P8 |
| 36 | P9 |
| 37 | PspnA |
| 38 | PspnAv2 |
| 39 | PspnF |
| 40 | PspnG |
| 41 | PspnQ |
| 42 | PspnQv2 |
| 43 | P21_mutant |
| 44 | P1_core |
| 45 | P1(−33) |
| 46 | P1 + ribswtch |
| 47 | P21-P1 |
| 48 | P1-P21 |
| 49 | P1765 |
| 50 | P3747 |
| 51 | P5078 |
| 52 | P7419 |
| 53 | P7156 (P3) |
| 54 | P7256 |
| 55 | P1941 |
| 56 | P3405 (P8) |
| 57 | P3407 |
| 58 | P2428 |
| 59 | P0927 |
| 60 | P0889 |
| 61 | P0186 |
| 62 | P3702_v2 |
| 63 | P7156_v2 |
| 64 | P7256_v2 |
| 65 | P1765_v2 |
| 66 | P7539_v2 |
| 67 | P7276_v2 |
| 68 | P0941_v2 |
| 69 | P0889_v2 |

In some embodiments, the promoters of the present disclosure exhibit at least 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% sequence identity with a promoter from the above Table 1.

Terminator Ladders

In some embodiments, the present disclosure teaches methods of improving genetically engineered host strains by providing one or more transcriptional termination sequences at a position 3′ to the end of the RNA encoding element. In some embodiments, the present disclosure teaches that the addition of termination sequences improves the efficiency of RNA transcription of a selected gene in the genetically engineered host. In other embodiments, the present disclosure teaches that the addition of termination sequences reduces the efficiency of RNA transcription of a selected gene in the genetically engineered host. Thus in some embodiments, the terminator ladders of the present disclosure comprises a series of terminator sequences exhibiting a range of transcription efficiencies (e.g., one weak terminator, one average terminator, and one strong promoter).

A transcriptional termination sequence may be any nucleotide sequence, which when placed transcriptionally downstream of a nucleotide sequence encoding an open reading frame, causes the end of transcription of the open reading frame. Such sequences are known in the art and may be of prokaryotic, eukaryotic or phage origin. Examples of terminator sequences include, but are not limited to, PTH-terminator, pET-T7 terminator, T3-T_{ϕ} terminator, pBR322-P4 terminator, vesicular stomatitus virus terminator, rrnB-T1 terminator, rrnC terminator, TTadc transcriptional terminator, and yeast-recognized termination sequences, such as Matα (α-factor) transcription terminator, native α-factor transcription termination sequence, ADR1transcription termination sequence, ADH2transcription termination sequence, and GAPD transcription termination sequence. A non-exhaustive listing of transcriptional terminator sequences may be found in the iGEM registry, which is available at: http://partsregistry.org/Terminators/Catalog.

In some embodiments, transcriptional termination sequences may be polymerase-specific or nonspecific, however, transcriptional terminators selected for use in the present embodiments should form a ‘functional combination’ with the selected promoter, meaning that the terminator sequence should be capable of terminating transcription by the type of RNA polymerase initiating at the promoter. For example, in some embodiments, the present disclosure teaches a eukaryotic RNA pol II promoter and eukaryotic RNA pol II terminators, a T7 promoter and T7 terminators, a T3 promoter and T3 terminators, a yeast-recognized promoter and yeast-recognized termination sequences, etc., would generally form a functional combination. The identity of the transcriptional termination sequences used may also be selected based on the efficiency with which transcription is terminated from a given promoter. For example, a heterologous transcriptional terminator sequence may be provided transcriptionally downstream of the RNA encoding element to achieve a termination efficiency of at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% from a given promoter.

In some embodiments, efficiency of RNA transcription from the engineered expression construct can be improved by providing nucleic acid sequence forms a secondary structure comprising two or more hairpins at a position 3′ to the end of the RNA encoding element. Not wishing to be bound by a particular theory, the secondary structure destabilizes the transcription elongation complex and leads to the polymerase becoming dissociated from the DNA template, thereby minimizing unproductive transcription of non-functional sequence and increasing transcription of the desired RNA. Accordingly, a termination sequence may be provided that forms a secondary structure comprising two or more adjacent hairpins. Generally, a hairpin can be formed by a palindromic nucleotide sequence that can fold back on itself to form a paired stem region whose arms are connected by a single stranded loop. In some embodiments, the termination sequence comprises 2, 3, 4, 5, 6, 7, 8, 9, 10 or more adjacent hairpins. In some embodiments, the adjacent hairpins are separated by 0, 1, 2, 3, 4, 5, 6, 7, 8, 9, 10, 11, 12, 13, 14, or 15 unpaired nucleotides. In some embodiments, a hairpin stem comprises 4, 5, 6, 7, 8, 9, 10, 11, 12, 13, 14, 15, 16, 17, 18, 19, 20, 21, 22, 23, 24, 25, 26, 27, 28, 29, 30 or more base pairs in length. In certain embodiments, a hairpin stem is 12 to 30 base pairs in length. In certain embodiments, the termination sequence comprises two or more medium-sized hairpins having stem region comprising about 9 to 25 base pairs. In some embodiments, the hairpin comprises a loop-forming region of 1, 2, 3, 4, 5, 6, 7, 8, 9, or 10 nucleotides. In some embodiments, the loop-forming region comprises 4-8 nucleotides. Not wishing to be bound by a particular theory, stability of the secondary structure can be correlated with termination efficiency. Hairpin stability is determined by its length, the number of mismatches or bulges it contains and the base composition of the paired region. Pairings between guanine and cytosine have three hydrogen bonds and are more stable compared to adenine-thymine pairings, which have only two. The G/C content of a hairpin-forming palindromic nucleotide sequence can be at least 60%, at least 65%, at least 70%, at least 75%, at least 80%, at least 85%, at least 90% or more. In some embodiments, the G/C content of a hairpin-forming palindromic nucleotide sequence is at least 80%. In some embodiments, the termination sequence is derived from one or more transcriptional terminator sequences of prokaryotic, eukaryotic or phage origin. In some embodiments, a nucleotide sequence encoding a series of 4, 5, 6, 7, 8, 9, 10 or more adenines (A) are provided 3′ to the termination sequence.

In some embodiments, the present disclosure teaches the use of a series of tandem termination sequences. In some embodiments, the first transcriptional terminator sequence of a series of 2, 3, 4, 5, 6, 7, or more may be placed directly 3′ to the final nucleotide of the dsRNA encoding element or at a distance of at least 1-5, 5-10, 10-15, 15-20, 20-25, 25-30, 30-35, 35-40, 40-45, 45-50, 50-100, 100-150, 150-200, 200-300, 300-400, 400-500, 500-1,000 or more nucleotides 3′ to the final nucleotide of the dsRNA encoding element. The number of nucleotides between tandem transcriptional terminator sequences may be varied, for example, transcriptional terminator sequences may be separated by 0, 1, 2, 3, 4, 5, 6, 7, 8, 9, 10, 10-15, 15-20, 20-25, 25-30, 30-35, 35-40, 40-45, 45-50 or more nucleotides. In some embodiments, the transcriptional terminator sequences may be selected based on their predicted secondary structure as determined by a structure prediction algorithm. Structural prediction programs are well known in the art and include, for example, CLC Main Workbench.

Persons having skill in the art will recognize that the methods of the present disclosure are compatible with any termination sequence. In some embodiments, the present disclosure teaches use of annotated Saccharopolyspora spp. terminators. In other embodiments, the present disclosure teaches use of transcriptional terminator sequences found in the iGEM registry, which is available at: http://partsregistry.org/Terminators/Catalog. A non-exhaustive listing of transcriptional terminator sequences of the present disclosure is provided in Table 2 below.

TABLE 2
Non-exhaustive list of termination sequences of the present disclosure.

| | | | |
|---|---|---|---|
| Name | Description | Direction | Length |

E. coli

| | | | |
|---|---|---|---|
| BBa_B0010 | T1 from E. coli rrnB | Forward | 80 |
| BBa_B0012 | TE from coliphageT7 | Forward | 41 |
| BBa_B0013 | TE from coliphage T7 (+/−) | Forward | 47 |
| BBa_B0015 | double terminator (B0010-B0012) | Forward | 129 |
| BBa_B0017 | double terminator (B0010-B0010) | Forward | 168 |
| BBa_B0053 | Terminator (His) | Forward | 72 |
| BBa_B0055 | -- No description -- | | 78 |
| BBa_B1002 | Terminator (artificial, | Forward | 34 |
| | small, % T ~= 85%) | | |
| BBa_B1003 | Terminator (artificial, small, % T ~= 80) | Forward | 34 |
| BBa_B1004 | Terminator (artificial, small, % T ~= 55) | Forward | 34 |
| BBa_B1005 | Terminator (artificial, | Forward | 34 |
| | small, % T ~= 25% | | |
| BBa_B1006 | Terminator (artificial, large, % T ~> 90) | Forward | 39 |
| BBa_B1010 | Terminator (artificial, large, % T ~< 10) | Forward | 40 |
| BBa_I11013 | Modification of biobricks part BBa_B0015 | | 129 |
| BBa_I51003 | -- No description -- | | 110 |
| BBa_J61048 | [rnpB-T1] Terminator | Forward | 113 |
| BBa_K1392970 | Terminator + Tetr Promoter + T4 | | 623 |
| | Endolysin | | |
| BBa_K1486001 | Arabinose promoter + CpxR | Forward | 1924 |
| BBa_K1486005 | Arabinose promoter + sfGFP − CpxR | Forward | 2668 |
| | [Cterm] | | |
| BBa_K1486009 | CxpR & Split IFP1.4 [Nterm + Nterm] | Forward | 3726 |
| BBa_K780000 | Terminator for Bacillus subtilis | | 54 |
| BBa_K864501 | T22, P22 late terminator | Forward | 42 |
| BBa_K864600 | T0 (21 imm) transcriptional terminator | Forward | 52 |
| BBa_K864601 | Lambda t1 transcriptional terminator | Forward | |
| BBa_B0011 | LuxICDABEG (+/−) | Bidirectional | 46 |
| BBa_B0014 | double terminator (B0012-B0011) | Bidirectional | 95 |
| BBa_B0021 | LuxICDABEG (+/−), reversed | Bidirectional | 46 |
| BBa_B0024 | double terminator (B0012-B0011), | Bidirectional | 95 |
| | reversed | | |
| BBa_B0050 | Terminator (pBR322, +/−) | Bidirectional | 33 |
| BBa_B0051 | Terminator (yciA/tonA, +/−) | Bidirectional | 35 |
| BBa_B1001 | Terminator (artifical, small, % T ~= 90) | Bidirectional | 34 |
| BBa_B1007 | Terminator (artificial, large, % T ~= 80) | Bidirectional | 40 |
| BBa_B1008 | Terminator (artificial, large, % T ~= 70) | Bidirectional | 40 |
| BBa_B1009 | Terminator (artificial, | Bidirectional | 40 |
| | large, % T ~= 40%) | | |
| BBa_K187025 | terminator in pAB, BioBytes plasmid | | 60 |
| BBa_K259006 | GFP-Terminator | Bidirectional | 823 |
| BBa_B0020 | Terminator (Reverse B0010) | Reverse | 82 |
| BBa_B0022 | TE from coliphageT7, reversed | Reverse | 41 |
| BBa_B0023 | TE from coliphage T7, reversed | Reverse | 47 |
| BBa_B0025 | double terminator (B0015), reversed | Reverse | 129 |
| BBa_B0052 | Terminator (rrnC) | Forward | 41 |
| BBa_B0060 | Terminator (Reverse B0050) | Bidirectional | 33 |
| BBa_B0061 | Terminator (Reverse B0051) | Bidirectional | 35 |
| BBa_B0063 | Terminator (Reverse B0053) | Reverse | 72 |

Yeast and other Eukaryotes

| | | | |
|---|---|---|---|
| BBa_J63002 | ADH1 terminator from S. cerevisiae | Forward | 225 |
| BBa_K110012 | STE2 terminator | Forward | 123 |
| BBa_K1462070 | cyc1 | | 250 |
| BBa_K1486025 | ADH1 Terminator | Forward | 188 |
| BBa_K392003 | yeast ADH1 terminator | | 129 |
| BBa_K801011 | TEF1 yeast terminator | | 507 |
| BBa_K801012 | ADH1 yeast terminator | | 349 |
| BBa_Y1015 | CycE1 | | 252 |
| BBa_J52016 | eukaryotic -- derived from SV40 early | Forward | 238 |
| | poly A signal sequence | | |
| BBa_J63002 | ADH1 terminator from S. cerevisiae | Forward | 225 |
| BBa_K110012 | STE2 terminator | Forward | 123 |
| BBa_K1159307 | 35S Terminator of Cauliflower Mosaic | | 217 |
| | Virus (CaMV) | | |
| BBa_K1462070 | cyc1 | | 250 |
| BBa_K1484215 | nopaline synthase terminator | | 293 |
| BBa_K1486025 | ADH1 Terminator | Forward | 188 |
| BBa_K392003 | yeast ADH1 terminator | | 129 |
| BBa_K404108 | hGH terminator | | 481 |
| BBa_K404116 | hGH_[AAV2]-right-ITR | | 632 |
| BBa_K678012 | SV40 poly A, terminator for | | 139 |
| | mammalian cells | | |
| BBa_K678018 | hGH poly A, terminator for | | 635 |
| | mammalian cells | | |
| BBa_K678019 | BGH poly A, mammalian terminator | | 233 |
| BBa_K678036 | trpC terminator for Aspergillus | | 759 |
| | nidulans | | |
| BBa_K678037 | T1-motni, terminator for Aspergillus | | 1006 |
| | niger | | |
| BBa_K678038 | T2-motni, terminator for Aspergillus | | 990 |
| | niger | | |
| BBa_678039 | T3-motni, terminator for Aspergillus | | 889 |
| | niger | | |
| BBa_K801011 | TEF1 yeast terminator | | 507 |
| BBa_K801012 | ADH1 yeast terminator | | 349 |
| BBa_Y1015 | CycE1 | | 252 |

A non-exhaustive list of additional terminator sequences of the present disclosure is provided in the below Table 3. Each of the terminator sequences can be referred to as a heterologous terminator or heterologous terminator polynucleotide.

TABLE 3
Selected terminator sequences
of the present disclosure.

| | Associated | | | Size |
|---|---|---|---|---|
| ID | Gene | Sequence | Source | (bp) |
| T1 | (elongation | CCCGAACCTTCGGGG | S. | 37 |
| | factor | GCGGGCCCTCTTGCT | spinosa | |
| | tu) | TTTCAAT | | |
| | | (SEQ ID No. 70) | | |
| T2 | (Leucyl | CGGGCAATAATACGT | S. | 49 |
| | amino- | GCCCGGACGGTAGTG | spinosa | |
| | peptidase) | CGAGCACGAGGTGGG | | |
| | | TACG | | |
| | | (SEQ ID No. 71) | | |
| T3 | (cytochrome | AGTTTGTCGAACCGG | S. | 41 |
| | P450 | CGGCGTTCGCCGGcT | spinosa | |
| | hydroxylase) | TTACCTTGCGC | | |
| | | (SEQ ID No. 72) | | |
| T4 | (F0F1 ATP | GGTTTCTCGAACCAG | S. | 42 |
| | synthase | TGCTTTGCGTACTGG | spinosa | |
| | subunit | TTGTCGTTGCAG | | |
| | beta) | (SEQ ID No. 73) | | |
| T5 | (FAD-linked | CGGAGCCAGAGGGCG | S. | 37 |
| | oxido- | CCTGAGTGCCTGTTT | erythraea | |
| | reductase) | TTGATCC | | |
| | | (SEQ ID No. 74) | | |
| T6 | (phospho- | AAACGCCCCCGGCTC | S. | 39 |
| | ribosyl- | CGGCCGGGGGCgTTT | erythraea | |
| | transferase) | TTGGTTGTG | | |
| | | (SEQ ID No. 75) | | |
| T7 | (ATP-binding | AGACGCAGGAGGTCT | S. | 37 |
| | protein) | CGTGAGGGGCTTTTC | erythraea | |
| | | CGCGAGC | | |
| | | (SEQ ID No. 76) | | |
| T8 | SACE_0757 | CGTGTGACTTGTCCC | S. | 35 |
| | (50 s | ACTCGGGGTTTTTGT | erythraea | |
| | Ribosomal | CGCGA | | |
| | protein L32) | (SEQ ID No. 77) | | |
| T9 | (tRNA-Arg) | GGATTCGTCCGGCCG | S. | 39 |
| | | AGGCCAATCGGCTTT | erythraea | |
| | | TCGGGGCCC | | |
| | | (SEQ ID No. 78) | | |
| T11 | (lsr2) | GCTTTCGTCGGCCGG | S. | 38 |
| | | GAACGCCCTGGTGTT | erythraea | |
| | | TCTTACCG | | |
| | | (SEQ ID No. 79) | | |
| T12 | (AraC) | TTGGGTGGATTCACC | S. | 38 |
| | | CCTACCGGGTGTTTT | erythraea | |
| | | TCTCGGCT | | |
| | | (SEQ ID No. 80) | | |
| NoT | None | — | — | 0 |

In some embodiments, the terminator of the present disclosure exhibit at least 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% sequence identity with a terminator from the above Table 3.

Hypothesis-Driven Diversity Pools and Hill Climbing

The present disclosure teaches that the HTP genomic engineering methods of the present disclosure do not require prior genetic knowledge in order to achieve significant gains in host cell performance. Indeed, the present disclosure teaches methods of generating diversity pools via several functionally agnostic approaches, including random mutagenesis, and identification of genetic diversity among pre-existing host cell variants (e.g., such as the comparison between a wild type host cell and an industrial variant).

In some embodiments however, the present disclosure also teaches hypothesis-driven methods of designing genetic diversity mutations that will be used for downstream HTP engineering. That is, in some embodiments, the present disclosure teaches the directed design of selected mutations. In some embodiments, the directed mutations are incorporated into the engineering libraries of the present disclosure (e.g., SNP swap, PRO swap, STOP swap, transposon mutagenesis diversity libraries, ribosomal binding site diversity libraries, anti-metabolite selection/fermentation product resistance libraries).

In some embodiments, the present disclosure teaches the creation of directed mutations based on gene annotation, hypothesized (or confirmed) gene function, or location within a genome. The diversity pools of the present disclosure may include mutations in genes hypothesized to be involved in a specific metabolic or genetic pathway associated in the literature with increased performance of a host cell. In other embodiments, the diversity pool of the present disclosure may also include mutations to genes present in an operon associated with improved host performance. In yet other embodiments, the diversity pool of the present disclosure may also include mutations to genes based on algorithmic predicted function, or other gene annotation.

In some embodiments, the present disclosure teaches a “shell” based approach for prioritizing the targets of hypothesis-driven mutations. The shell metaphor for target prioritization is based on the hypothesis that only a handful of primary genes are responsible for most of a particular aspect of a host cell's performance (e.g., production of a single biomolecule). These primary genes are located at the core of the shell, followed by secondary effect genes in the second layer, tertiary effects in the third shell, and . . . etc. For example, in one embodiment the core of the shell might comprise genes encoding critical biosynthetic enzymes within a selected metabolic pathway (e.g., production of citric acid). Genes located on the second shell might comprise genes encoding for other enzymes within the biosynthetic pathway responsible for product diversion or feedback signaling. Third tier genes under this illustrative metaphor would likely comprise regulatory genes responsible for modulating expression of the biosynthetic pathway, or for regulating general carbon flux within the host cell.

The present disclosure also teaches “hill climb” methods for optimizing performance gains from every identified mutation. In some embodiments, the present disclosure teaches that random, natural, or hypothesis-driven mutations in HTP diversity libraries can result in the identification of genes associated with host cell performance. For example, the present methods may identify one or more beneficial SNPs located on, or near, a gene coding sequence. This gene might be associated with host cell performance, and its identification can be analogized to the discovery of a performance “hill” in the combinatorial genetic mutation space of an organism.

In some embodiments, the present disclosure teaches methods of exploring the combinatorial space around the identified hill embodied in the SNP mutation. That is, in some embodiments, the present disclosure teaches the perturbation of the identified gene and associated regulatory sequences in order to optimize performance gains obtained from that gene node (i.e., hill climbing). Thus, according to the methods of the present disclosure, a gene might first be identified in a diversity library sourced from random mutagenesis, but might be later improved for use in the strain improvement program through the directed mutation of another sequence within the same gene.

The concept of hill climbing can also be expanded beyond the exploration of the combinatorial space surrounding a single gene sequence. In some embodiments, a mutation in a specific gene might reveal the importance of a particular metabolic or genetic pathway to host cell performance. For example, in some embodiments, the discovery that a mutation in a single RNA degradation gene resulted in significant host performance gains could be used as a basis for mutating related RNA degradation genes as a means for extracting additional performance gains from the host organism. Persons having skill in the art will recognize variants of the above describe shell and hill climb approaches to directed genetic design. High-throughput Screening.

Cell Culture and Fermentation

Cells of the present disclosure can be cultured in conventional nutrient media modified as appropriate for any desired biosynthetic reactions or selections. In some embodiments, the present disclosure teaches culture in inducing media for activating promoters. In some embodiments, the present disclosure teaches media with selection agents, including selection agents of transformants (e.g., antibiotics), or selection of organisms suited to grow under inhibiting conditions (e.g., high ethanol conditions). In some embodiments, the present disclosure teaches growing cell cultures in media optimized for cell growth. In other embodiments, the present disclosure teaches growing cell cultures in media optimized for product yield. In some embodiments, the present disclosure teaches growing cultures in media capable of inducing cell growth and also contains the necessary precursors for final product production (e.g., high levels of sugars for ethanol production).

Culture conditions, such as temperature, pH and the like, are those suitable for use with the host cell selected for expression, and will be apparent to those skilled in the art. As noted, many references are available for the culture and production of many cells, including cells of bacterial, plant, animal (including mammalian) and archaebacterial origin. See e.g., Sambrook, Ausubel (all supra), as well as Berger, Guide to Molecular Cloning Techniques, Methods in Enzymology volume 152 Academic Press, Inc., San Diego, Calif.; and Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelle et al., (1989) In Vitro Cell Dev. Biol. 25:1016-1024, all of which are incorporated herein by reference. For plant cell culture and regeneration, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg N.Y.); Jones, ed. (1984) Plant Gene Transfer and Expression Protocols, Humana Press, Totowa, N.J. and Plant Molecular Biology (1993) R. R. D. Croy, Ed. Bios Scientific Publishers, Oxford, U.K. ISBN 0 12 198370 6, all of which are incorporated herein by reference. Cell culture media in general are set forth in Atlas and Parks (eds.) The Handbook of Microbiological Media (1993) CRC Press, Boca Raton, Fla., which is incorporated herein by reference. Additional information for cell culture is found in available commercial literature such as the Life Science Research Cell Culture Catalogue from Sigma-Aldrich, Inc (St Louis, Mo.) (“Sigma-LSRCCC”) and, for example, The Plant Culture Catalogue and supplement also from Sigma-Aldrich, Inc (St Louis, Mo.) (“Sigma-PCCS”), all of which are incorporated herein by reference.

The culture medium to be used must in a suitable manner satisfy the demands of the respective strains. Descriptions of culture media for various microorganisms are present in the “Manual of Methods for General Bacteriology” of the American Society for Bacteriology (Washington D.C., USA, 1981).

The present disclosure furthermore provides a process for fermentative preparation of a product of interest, comprising the steps of: a) culturing a microorganism according to the present disclosure in a suitable medium, resulting in a fermentation broth; and b) concentrating the product of interest in the fermentation broth of a) and/or in the cells of the microorganism.

In some embodiments, the present disclosure teaches that the microorganisms produced may be cultured continuously—as described, for example, in WO 05/021772—or discontinuously in a batch process (batch cultivation) or in a fed-batch or repeated fed-batch process for the purpose of producing the desired organic-chemical compound. A summary of a general nature about known cultivation methods is available in the textbook by Chmiel (Bioprozeßtechnik. 1: Einführung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)).

In some embodiments, the cells of the present disclosure are grown under batch or continuous fermentations conditions.

Classical batch fermentation is a closed system, wherein the compositions of the medium is set at the beginning of the fermentation and is not subject to artificial alternations during the fermentation. A variation of the batch system is a fed-batch fermentation which also finds use in the present disclosure. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art.

Continuous fermentation is a system where a defined fermentation medium is added continuously to a bioreactor and an equal amount of conditioned medium is removed simultaneously for processing and harvesting of desired biomolecule products of interest. In some embodiments, continuous fermentation generally maintains the cultures at a constant high density where cells are primarily in log phase growth. In some embodiments, continuous fermentation generally maintains the cultures at a stationary or late log/stationary, phase growth. Continuous fermentation systems strive to maintain steady state growth conditions.

Methods for modulating nutrients and growth factors for continuous fermentation processes as well as techniques for maximizing the rate of product formation are well known in the art of industrial microbiology.

For example, a non-limiting list of carbon sources for the cultures of the present disclosure include, sugars and carbohydrates such as, for example, glucose, sucrose, lactose, fructose, maltose, molasses, sucrose-containing solutions from sugar beet or sugar cane processing, starch, starch hydrolysate, and cellulose; oils and fats such as, for example, soybean oil, sunflower oil, groundnut oil and coconut fat; fatty acids such as, for example, palmitic acid, stearic acid, and linoleic acid; alcohols such as, for example, glycerol, methanol, and ethanol; and organic acids such as, for example, acetic acid or lactic acid.

A non-limiting list of the nitrogen sources for the cultures of the present disclosure include, organic nitrogen-containing compounds such as peptones, yeast extract, meat extract, malt extract, corn steep liquor, soybean flour, and urea; or inorganic compounds such as ammonium sulfate, ammonium chloride, ammonium phosphate, ammonium carbonate, and ammonium nitrate. The nitrogen sources can be used individually or as a mixture.

A non-limiting list of the possible phosphorus sources for the cultures of the present disclosure include, phosphoric acid, potassium dihydrogen phosphate or dipotassium hydrogen phosphate or the corresponding sodium-containing salts.

The culture medium may additionally comprise salts, for example in the form of chlorides or sulfates of metals such as, for example, sodium, potassium, magnesium, calcium and iron, such as, for example, magnesium sulfate or iron sulfate, which are necessary for growth.

Finally, essential growth factors such as amino acids, for example homoserine and vitamins, for example thiamine, biotin or pantothenic acid, may be employed in addition to the abovementioned substances.

In some embodiments, the pH of the culture can be controlled by any acid or base, or buffer salt, including, but not limited to sodium hydroxide, potassium hydroxide, ammonia, or aqueous ammonia; or acidic compounds such as phosphoric acid or sulfuric acid in a suitable manner. In some embodiments, the pH is generally adjusted to a value of from 6.0 to 8.5, preferably 6.5 to 8.

In some embodiments, the cultures of the present disclosure may include an anti-foaming agent such as, for example, fatty acid polyglycol esters. In some embodiments the cultures of the present disclosure are modified to stabilize the plasmids of the cultures by adding suitable selective substances such as, for example, antibiotics.

In some embodiments, the culture is carried out under aerobic conditions. In order to maintain these conditions, oxygen or oxygen-containing gas mixtures such as, for example, air are introduced into the culture. It is likewise possible to use liquids enriched with hydrogen peroxide. The fermentation is carried out, where appropriate, at elevated pressure, for example at an elevated pressure of from 0.03 to 0.2 MPa. The temperature of the culture is normally from 20° C. to 45° C. and preferably from 25° C. to 40° C., particularly preferably from 30° C. to 37° C. In batch or fed-batch processes, the cultivation is preferably continued until an amount of the desired product of interest (e.g. an organic-chemical compound) sufficient for being recovered has formed. This aim can normally be achieved within 10 hours to 160 hours. In continuous processes, longer cultivation times are possible. The activity of the microorganisms results in a concentration (accumulation) of the product of interest in the fermentation medium and/or in the cells of said microorganisms.

In some embodiments, the culture is carried out under anaerobic conditions.

Screening

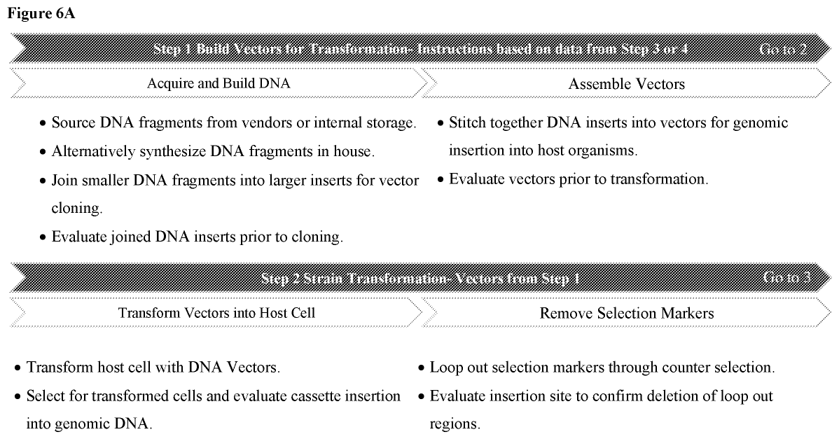

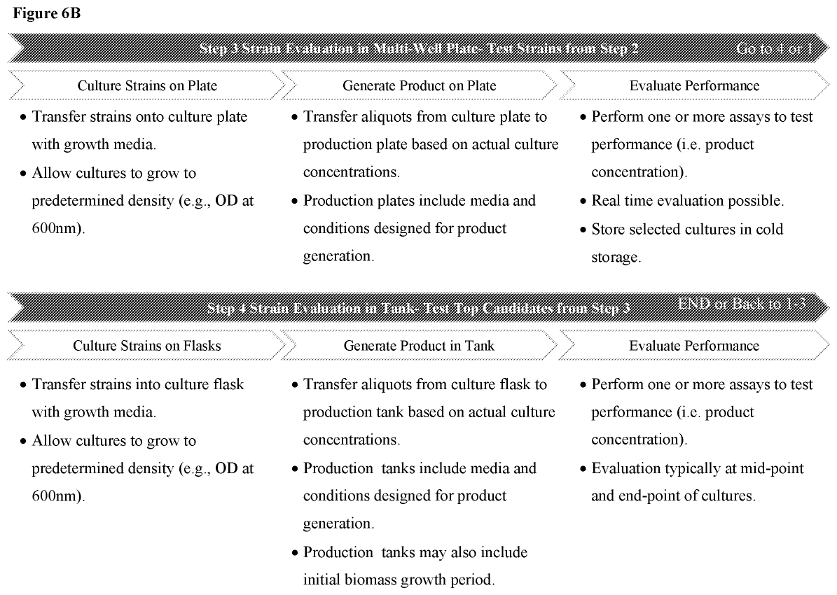

In some embodiments, the present disclosure teaches high-throughput initial screenings. In other embodiments, the present disclosure also teaches robust tank-based validations of performance data (see FIG. 6B).

In some embodiments, the high-throughput screening process is designed to predict performance of strains in bioreactors. As previously described, culture conditions are selected to be suitable for the organism and reflective of bioreactor conditions. Individual colonies are picked and transferred into 96 well plates and incubated for a suitable amount of time. Cells are subsequently transferred to new 96 well plates for additional seed cultures, or to production cultures. Cultures are incubated for varying lengths of time, where multiple measurements may be made. These may include measurements of product, biomass or other characteristics that predict performance of strains in bioreactors. High-throughput culture results are used to predict bioreactor performance.

In some embodiments, the tank-based performance validation is used to confirm performance of strains isolated by high throughput screening. Candidate strains are screened using bench scale fermentation reactors for relevant strain performance characteristics such as productivity or yield.

### Product Recovery and Quantification

Methods for screening for the production of products of interest are known to those of skill in the art and are discussed throughout the present specification. Such methods may be employed when screening the strains of the disclosure.

In some embodiments, the present disclosure teaches methods of improving strains designed to produce non-secreted intracellular products. For example, the present disclosure teaches methods of improving the robustness, yield, efficiency, or overall desirability of cell cultures producing intracellular enzymes, oils, pharmaceuticals, or other valuable small molecules or peptides. The recovery or isolation of non-secreted intracellular products can be achieved by lysis and recovery techniques that are well known in the art, including those described herein.

For example, in some embodiments, cells of the present disclosure can be harvested by centrifugation, filtration, settling, or other method. Harvested cells are then disrupted by any convenient method, including freeze-thaw cycling, sonication, mechanical disruption, or use of cell lysing agents, or other methods, which are well known to those skilled in the art.

The resulting product of interest, e.g. a polypeptide, may be recovered/isolated and optionally purified by any of a number of methods known in the art. For example, a product polypeptide may be isolated from the nutrient medium by conventional procedures including, but not limited to: centrifugation, filtration, extraction, spray-drying, evaporation, chromatography (e.g., ion exchange, affinity, hydrophobic interaction, chromatofocusing, and size exclusion), or precipitation. Finally, high performance liquid chromatography (HPLC) can be employed in the final purification steps. (See for example Purification of intracellular protein as described in Parry et al., 2001, Biochem. J. 353:117, and Hong et al., 2007, Appl. Microbiol. Biotechnol. 73:1331, both incorporated herein by reference).

In addition to the references noted supra, a variety of purification methods are well known in the art, including, for example, those set forth in: Sandana (1997) Bioseparation of Proteins, Academic Press, Inc.; Bollag et al. (1996) Protein Methods, 2^{nd}, Edition, Wiley-Liss, NY; Walker (1996) The Protein Protocols Handbook Humana Press, NJ; Harris and Angal (1990) Protein Purification Applications: A Practical Approach, IRL Press at Oxford, Oxford, England; Harris and Angal Protein Purification Methods: A Practical Approach, IRL Press at Oxford, Oxford, England; Scopes (1993) Protein Purification: Principles and Practice 3^{rd} Edition, Springer Verlag, NY; Janson and Ryden (1998) Protein Purification: Principles, High Resolution Methods and Applications, Second Edition, Wiley-VCH, NY; and Walker (1998) Protein Protocols on CD-ROM, Humana Press, NJ, all of which are incorporated herein by reference.

In some embodiments, the present disclosure teaches the methods of improving strains designed to produce secreted products. For example, the present disclosure teaches methods of improving the robustness, yield, efficiency, or overall desirability of cell cultures producing valuable small molecules or peptides.

In some embodiments, immunological methods may be used to detect and/or purify secreted or non-secreted products produced by the cells of the present disclosure. In one example approach, antibody raised against a product molecule (e.g., against an insulin polypeptide or an immunogenic fragment thereof) using conventional methods is immobilized on beads, mixed with cell culture media under conditions in which the endoglucanase is bound, and precipitated. In some embodiments, the present disclosure teaches the use of enzyme-linked immunosorbent assays (ELISA).

In other related embodiments, immunochromatography is used, as disclosed in U.S. Pat. Nos. 5,591,645, 4,855,240, 4,435,504, 4,980,298, and Se-Hwan Paek, et al., “Development of rapid One-Step Immunochromatographic assay, Methods”, 22, 53-60, 2000), each of which are incorporated by reference herein. A general immunochromatography detects a specimen by using two antibodies. A first antibody exists in a test solution or at a portion at an end of a test piece in an approximately rectangular shape made from a porous membrane, where the test solution is dropped. This antibody is labeled with latex particles or gold colloidal particles (this antibody will be called as a labeled antibody hereinafter). When the dropped test solution includes a specimen to be detected, the labeled antibody recognizes the specimen so as to be bonded with the specimen. A complex of the specimen and labeled antibody flows by capillarity toward an absorber, which is made from a filter paper and attached to an end opposite to the end having included the labeled antibody. During the flow, the complex of the specimen and labeled antibody is recognized and caught by a second antibody (it will be called as a tapping antibody hereinafter) existing at the middle of the porous membrane and, as a result of this, the complex appears at a detection part on the porous membrane as a visible signal and is detected.

In some embodiments, the screening methods of the present disclosure are based on photometric detection techniques (absorption, fluorescence). For example, in some embodiments, detection may be based on the presence of a fluorophore detector such as GFP bound to an antibody. In other embodiments, the photometric detection may be based on the accumulation on the desired product from the cell culture. In some embodiments, the product may be detectable via UV of the culture or extracts from said culture.

Persons having skill in the art will recognize that the methods of the present disclosure are compatible with host cells producing any desirable biomolecule product of interest. Table 4 below presents a non-limiting list of the product categories, biomolecules, and host cells, included within the scope of the present disclosure. These examples are provided for illustrative purposes, and are not meant to limit the applicability of the presently disclosed technology in any way.

TABLE 4
A non-limiting list of the host cells and products of interest of the present disclosure.

| Product category | Products | Host category | Hosts |
|---|---|---|---|
| Amino acids | Lysine | Bacteria | Corynebacterium glutamicum |
| Amino acids | Methionine | Bacteria | Escherichia coli |
| Amino acids | MSG | Bacteria | Corynebacterium glutamicum |
| Amino acids | Threonine | Bacteria | Escherichia coli |
| Amino acids | Threonine | Bacteria | Corynebacterium glutamicum |
| Amino acids | Tryptophan | Bacteria | Corynebacterium glutamicum |
| Enzymes | Enzymes (11) | Filamentous | Trichoderma reesei |
| | | fungi | |
| Enzymes | Enzymes (11) | Fungi | Myceliopthora thermophila |
| | | | (C1) |
| Enzymes | Enzymes (11) | Filamentous | Aspergillus oryzae |
| | | fungi | |
| Enzymes | Enzymes (11) | Filamentous | Aspergillus niger |
| | | fungi | |
| Enzymes | Enzymes (11) | Bacteria | Bacillus subtilis |
| Enzymes | Enzymes (11) | Bacteria | Bacillus licheniformis |
| Enzymes | Enzymes (11) | Bacteria | Bacillus clausii |
| Flavor & | Agarwood | Yeast | Saccharomyces cerevisiae |
| Fragrance | | | |
| Flavor & | Ambrox | Yeast | Saccharomyces cerevisiae |
| Fragrance | | | |
| Flavor & | Nootkatone | Yeast | Saccharomyces cerevisiae |
| Fragrance | | | |
| Flavor & | Patchouli oil | Yeast | Saccharomyces cerevisiae |
| Fragrance | | | |
| Flavor & | Saffron | Yeast | Saccharomyces cerevisiae |
| Fragrance | | | |
| Flavor & | Sandalwood oil | Yeast | Saccharomyces cerevisiae |
| Fragrance | | | |
| Flavor & | Valencene | Yeast | Saccharomyces cerevisiae |
| Fragrance | | | |
| Flavor & | Vanillin | Yeast | Saccharomyces cerevisiae |
| Fragrance | | | |
| Food | CoQ10/Ubiquinol | Yeast | Schizosaccharomyces pombe |
| Food | Omega 3 fatty | Microalgae | Schizochytrium |
| | acids | | |
| Food | Omega 6 fatty | Microalgae | Schizochytrium |
| | acids | | |
| Food | Vitamin B12 | Bacteria | Propionibacterium |
| | | | freudenreichii |
| Food | Vitamin B2 | Filamentous | Ashbya gossypii |
| | | fungi | |
| Food | Vitamin B2 | Bacteria | Bacillus subtilis |
| Food | Erythritol | Yeast-like | Torula coralline |
| | | fungi | |
| Food | Erythritol | Yeast-like | Pseudozyma tsukubaensis |
| | | fungi | |
| Food | Erythritol | Yeast-like | Moniliella pollinis |
| | | fungi | |
| Food | Steviol | Yeast | Saccharomyces cerevisiae |
| | glycosides | | |
| Hydrocolloids | Diutan gum | Bacteria | Sphingomonassp |
| Hydrocolloids | Gellan gum | Bacteria | Sphingomonas elodea |
| Hydrocolloids | Xanthan gum | Bacteria | Xanthomonas campestris |
| Intermediates | 1,3-PDO | Bacteria | Escherichia coli |
| Intermediates | 1,4-BDO | Bacteria | Escherichia coli |
| Intermediates | Butadiene | Bacteria | Cupriavidus necator |
| Intermediates | n-butanol | Bacteria | Clostridium acetobutylicum |
| | | (obligate | |
| | | anaerobe) | |
| Organic acids | Citric acid | Filamentous | Aspergillus niger |
| | | fungi | |
| Organic acids | Citric acid | Yeast | Pichia guilliermondii |
| Organic acids | Gluconic acid | Filamentous | Aspergillus niger |
| | | fungi | |
| Organic acids | Itaconic acid | Filamentous | Aspergillus terreus |
| | | fungi | |
| Organic acids | Lactic acid | Bacteria | Lactobacillus |
| Organic acids | Lactic acid | Bacteria | Geobacillus |
| | | | thermoglucosidasius |
| Organic acids | LCDAs- | Yeast | Candida |
| | DDDA | | |
| Polyketides/Ag | Spinosad | Bacteria | Saccharopolyspora spinosa |
| Polyketides/Ag | Spinetoram | Bacteria | Saccharopolyspora spinosa |
| isoflavone | genistein | Bacteria | Saccharopolyspora erythraea |
| Enzymes | choline oxidase | Bacteria | Streptomyces, |
| | | | Thermoactinomyces or |
| | | | Saccharopolyspora |
| Pharmaceutical | Coumamidine | Bacteria | Saccharopolyspora sp. |
| composition | compounds | | |
| inhibitor of nematode | ivermectin | Bacteria | Saccharopolyspora erythraea |
| larval development | aglycone | | |
| inhibitor of enzyme | HMG-CoA | Bacteria | Saccharopolyspora sp. |
| | reductase | | |
| | inhibitors | | |
| Organic acids | carboxylic acid | Bacteria | Saccharopolyspora hirsuta |
| | isomers | | |
| antibiotic | Erythromycin | Bacteria | Saccharopolyspora erythraea |

In some embodiments, the host cell is a Saccharopolyspora sp. In some embodiments, the Saccharopolyspora sp is a Saccharopolyspora spinosa strain. Products of interest produced in Saccharopolyspora spp. is provided in Table 4.1 below.

TABLE 4.1
A non-limiting list of products of interest in Saccharopolyspora spp. of the present
disclosure

| Product name | Structure |
|---|---|
| Spinosyn A | 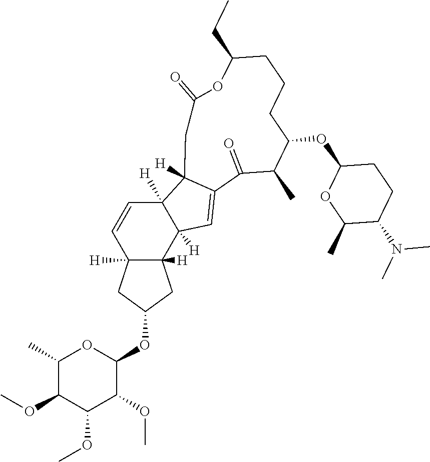 |
| Spinosyn B | 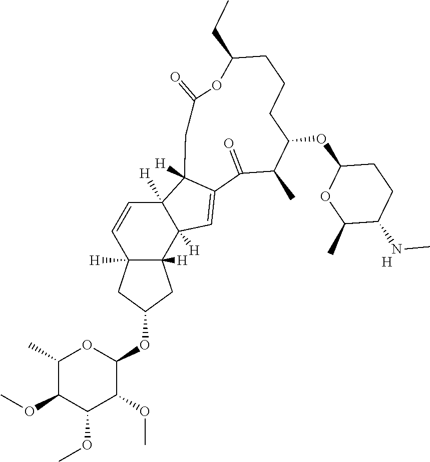 |
| Spinosyn C | 4″-di-N-demethyl-spinosyn A |
| Spinosyn D | 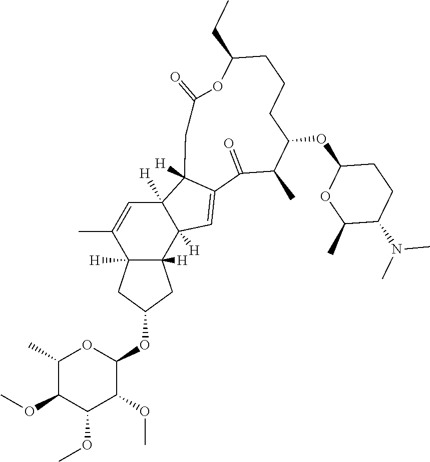 |
| Spinosyn E | 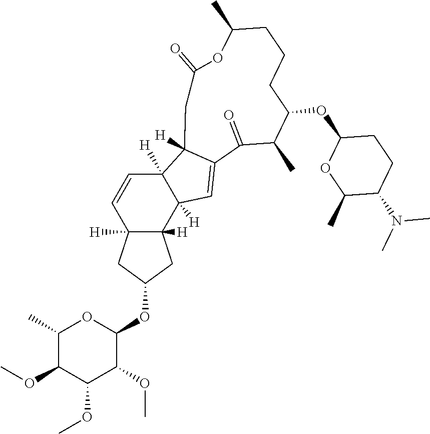 |
| Spinosyn F | 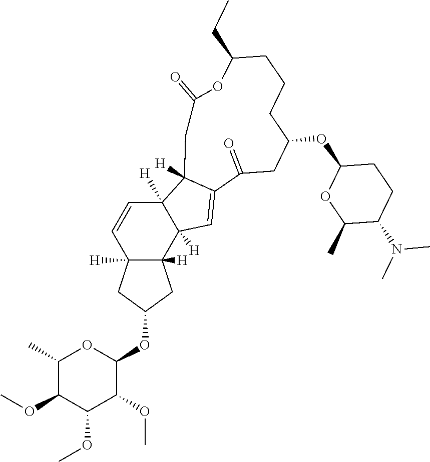 |
| Spinosyn G | 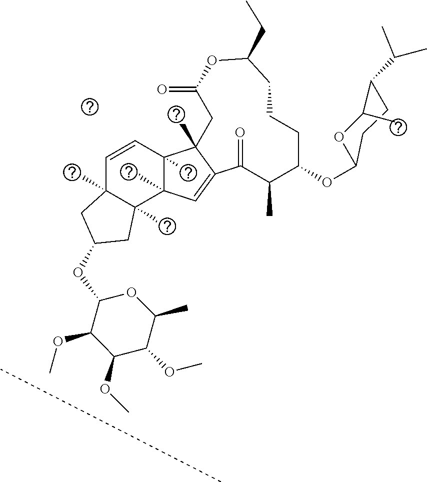 |
| Spinosyn H | 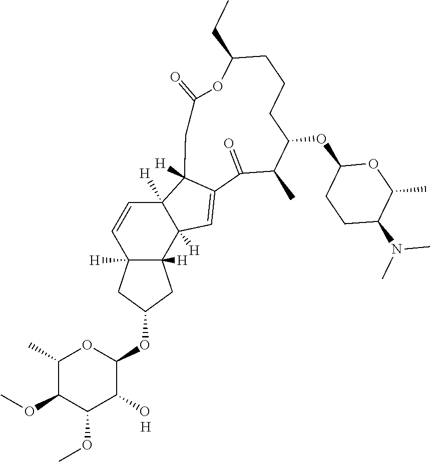 |
| Spinosyn I | N/A |
| Spinosyn J | 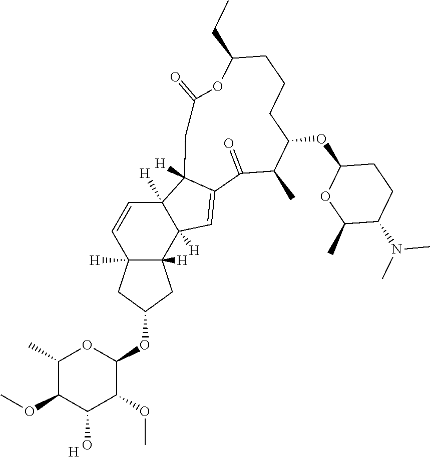 |
| Spinosyn K | 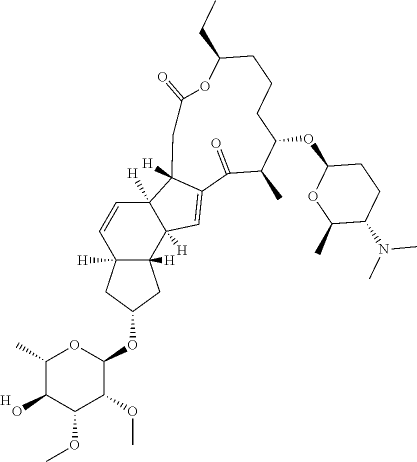 |
| Spinosyn L | 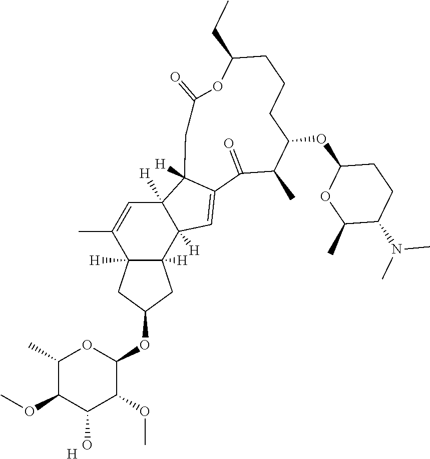 |
| Spinosyn M | 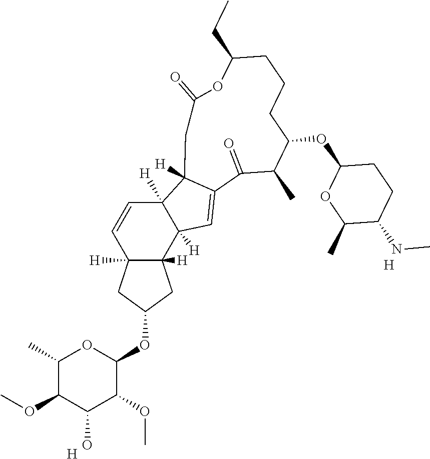 |
| Spinosyn N | 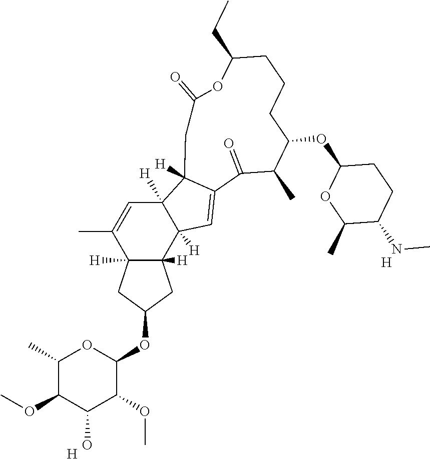 |
| Spinosyn O | 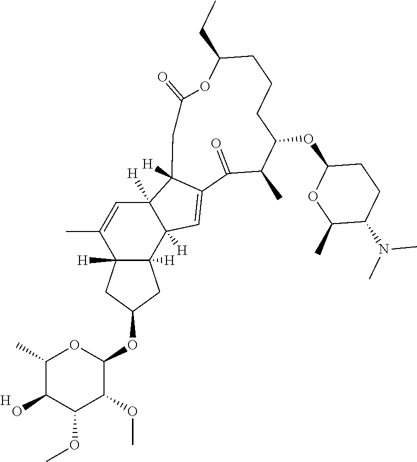 |
| Spinosyn P | 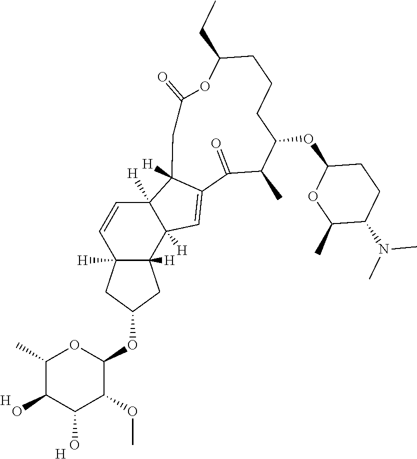 |
| Spinosyn Q | 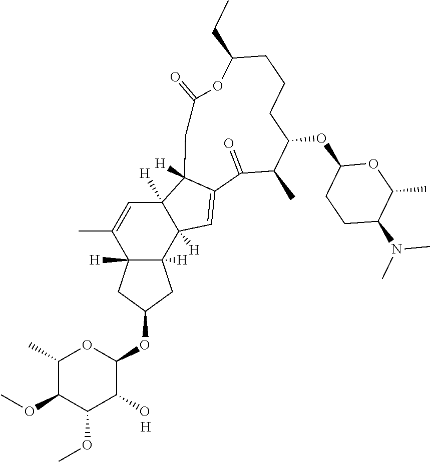 |
| Spinosyn R | 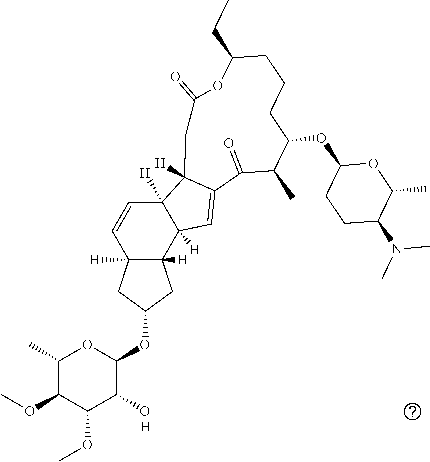 |
| Spinosyn S | 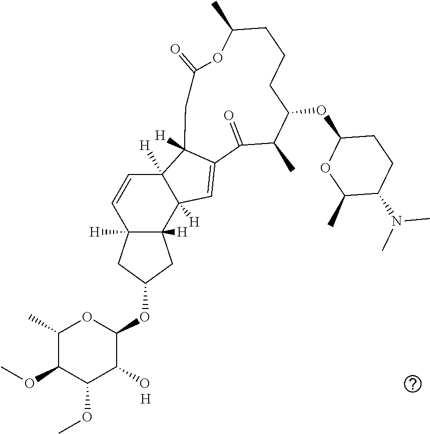 |
| Spinosyn T | 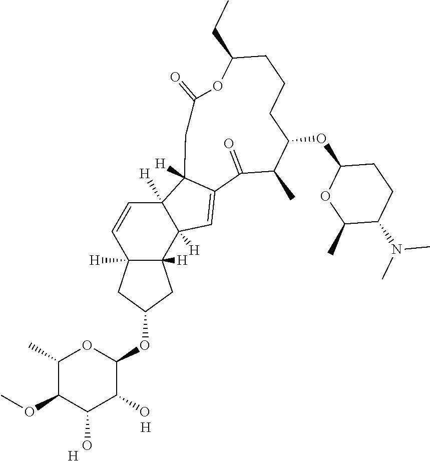 |
| Spinosyn U | 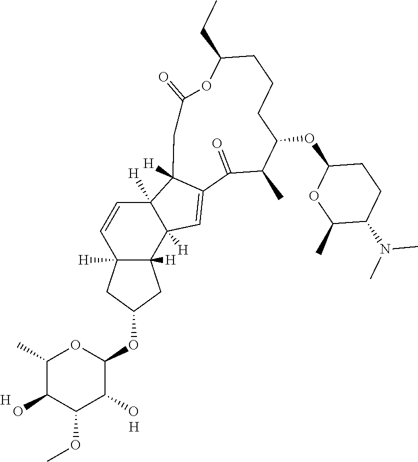 |
| Spinosyn V | 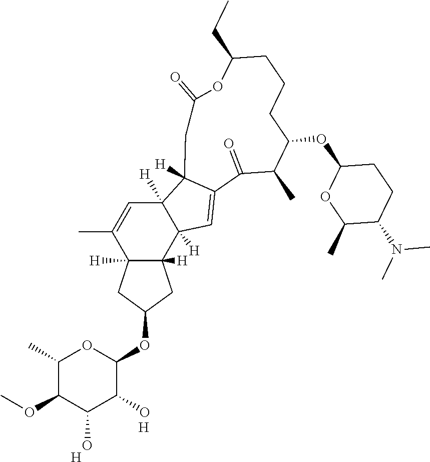 |
| Spinosyn W | 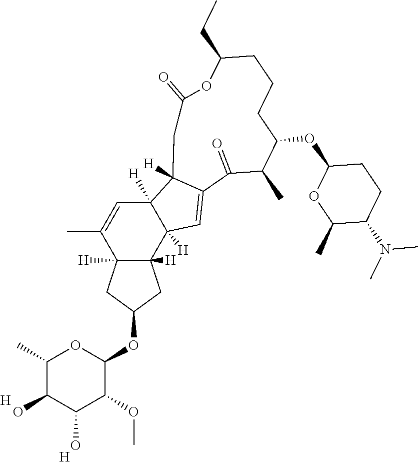 |
| Spinosyn X | N/A |
| Spinosyn Y | 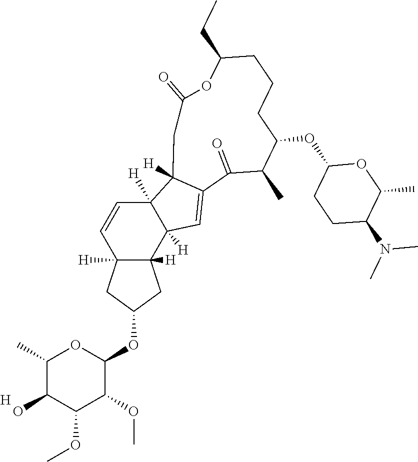 |

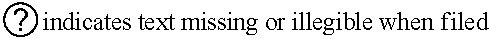

The spinosyns are a large family of unprecedented compounds produced from fermentation of two species of Saccharopolyspora. Their core structure is a polyketide-derived tetracyclic macrolide appended with two saccharides. They show potent insecticidal activities against many commercially significant species that cause extensive damage to crops and other plants. They also show activity against important external parasites of livestock, companion animals and human S. spinosa d is a defined combination of the two principal fermentation factors, spinosyns A and D. Both spinosyn A and spinosyn D are the two most abundant fermentation components for S. spinosa. Structure-activity relationships (SARs) have been extensively studied, leading to development of a semisynthetic second-generation derivative, spinetoram (Kirst, The Journal of Antibiotics (2010) 63, 101-111). Numerous structurally related compounds from various spinosyn fermentations have now been isolated and identified. Their structures fall into several general categories of single-type changes in the aglycone or saccharides of spinosyn A. Some factors have either one additional or one missing C-methyl group relative to spinosyn A, which would occur biosynthetically by interchanges of acetate and propionate at appropriate times during formation of the polyketide framework. In addition to spinosyn D (6-methyl-spinosyn A), other single C-methyl-modified factors include spinosyn E (16-demethyl-spinosyn A) and spinosyn F (22-demethyl-spinosyn A). Modifications of the two saccharides include spinosyn H (2′-O-demethyl-spinosyn A), spinosyn J (3′-O-demethyl-spinosyn A), spinosyn B (4″-N-demethyl-spinosyn A) and spinosyn C (4″-di-N-demethyl-spinosyn A). Another structural change is replacement of the aminosugar, D-forosamine, by a different saccharide such as L-ossamine (spinosyn G). In recent years, the spinosad biosynthetic pathway has been clarified more accuracy: spnA, spnB, spnC, spnD, and spnE responsible for type I polyketide synthase; spnF, spnJ, spnL, and spnM for modifying the polyketide synthase product (Kim et al., “Enzyme-catalysed 4+2 cycloaddition is a key step in the biosynthesis of spinosyn A”. Nature. 2011, 473: 109-112); spnG, spnH, spnI, and spnK for rhamnose attachment and methylation (Kim et al., “Biosynthesis of spinosyn in Saccharopolyspora spinosa: synthesis of permethylated rhamnose and characterization of the functions of SpnH, SpnI and SpnK.” J Am Chem Soc. 2010, 132: 2901-2903); spnP, spnO, spnN, spnQ, spnR, and spnS for forosamine biosynthesis; gtt, gdh, epi, and kre for rhamnose biosynthesis (Madduri et al. “Rhamnose biosynthesis pathway supplies precursors for primary and secondary metabolism in Saccharopolyspora spinosa.” J Bacteriol. 2001, 183: 5632-5638) and beside the spinosad gene cluster four genes ORF-L16, ORF-R1, and ORF-R2, have no effect on spinosad biosynthesis. These genes are among the potential targets of the genetic engineering methods described herein. Additional genes involved in spinosyn synthesis are described in U.S. Pat. Nos. 7,626,010, 8,624,009, which is herein incorporated by reference in its entirety for all purposes.

Spinetoram is a chemically modified spinosyns J/L mixture. The mixture comprises two primary factors 3′-O-ethyl-5,6-dihydro spinosyns J, and 3′O-ethyl spinosyns L. Spinetoram has broader spectrum and more potent compared to spinosad, and has improved residual activity in the field. The creation of spinetoram is a result of an artificial neural network (ANN) based strategy in which molecule designs employs software that mimics neural connections in the mammalian brain to recognize patterns and can be used to estimate activities of suggested molecular modifications. Consequently, it was found that certain alkyl substitution patterns on the rhamnose moiety, in particular the 2′,3′, 4′-tir-O-ethyl spinosyns A analog would represent a promising modification. Further, it was indicated that rhamnose-3′-O-ethylation would represent the major contributor to activity enhancement over 2′- or 4′-O-ethylations. Ultimately, spinetoram was created (Sparks et al., 2008, Neural network-based QSAR and insecticide discovery: spinetoram. J Comput Aid Mol Des 22:393-401. doi:10.1007/s10822-008-9205-8).

In some embodiments, the product of interest is spinosad. Spinosad is a novel mode-of-action insecticide derived from a family of natural products obtained by fermentation of S. spinosa. Spinosyns occur in over 20 natural forms, and over 200 synthetic forms (spinosoids) have been produced in the lab (Watson, Gerald (31 May 2001). “Actions of Insecticidal Spinosyns on gama-Aminobutyric Acid Responses for Small-Diameter Cockroach Neurons”. Pesticide Biochemistry and Physiology. 71: 20-28, incorporated by reference in its entirety). Spinosad contains a mix of two spinosoids, spinosyn A, the major component, and spinosyn D (the minor component), in a roughly 17:3 ratio.

In some embodiments, molecules that can be used to screen for mutant Saccharopolyspora strains include, but are not limited to: 1) molecules involved in the spinosyn synthesis pathway (e.g., a spinosyn); 2) molecules involved in the SAM/methionine pathway (e.g., alpha-methyl methionine (aMM) or norleucine); 3) molecules involved in the lysine production pathway (e.g., thialysine or a mixture of alpha-ketobytarate and aspartate hydoxymate); 4) molecules involved in the tryptophan pathway (e.g., azaserine or 5-fuoroindole); 5) molecules involved in the threonine pathway (e.g., beta-hydroxynorvaline); 6) molecules involved in the acetyl-CoA production pathway (e.g., cerulenin); and 7) molecules involved in the de-novo or salvage purine and pyrimidine pathways (e.g., purine or a pyrimidine analogs).

In some embodiments, the concentration of the spinosyn used for screening is about 10 μg/ml, 20 μg/ml, 30 μg/ml, 40 μg/ml, 50 μg/ml, 60 μg/ml, 70 μg/ml, 80 μg/ml, 90 μg/ml, 100 μg/ml, 200 μg/ml, 300 μg/ml, 400 μg/ml, 500 μg/ml, 600 μg/ml, 700 μg/ml, 800 μg/ml, 900 μg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, or more.

In some embodiments, the concentration of aMM used for screening is about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, or more.

In some embodiments, the exact concentration of a molecule used for screening may be empirically determined, depending on the strain used. In general, base strains would be more sensitive than strains that have been engineered.

Genetic tools, resources, compositions, methods, and strains for Saccharopolyspora spp. can be found in U.S. Pat. Nos. 6,960,453, 6,270,768, 5,631,155, 5,670,364, 5,554,519, 5,187,088, 5,202,242, 6,616,953, 5,171,740, 6,420,177, 8,624,009, 7,626,010, 5,124,258, 5,362,634, 6,043,064, 4,293,651, 4,389,486, 6,627,427, 5,663,067, 5,081,023, 6,780,633, 6,004,787, 6,365,399, 5,801,032, 8,741,603, 4,328,307, 4,425,430, 7,022,526, 5,234,828, 5,786,181, 5,153,128, 8,841,092, 4,251,511, 9,309,524, 6,437,151, 5,908,764,8,911,970, 5,824,513, 6,524,841, 7,198,922, 6,200,813, 9,334,514, 5,496,931, 7,630,836, 5,198,360, 6,710,189, 6,251,636, 7,807,418, 6,780,620, 6,500,960, and 7,459,294, each of which is herein incorporated by reference in its entirety for all purposes.

### Selection Criteria and Goals

The selection criteria applied to the methods of the present disclosure will vary with the specific goals of the strain improvement program. The present disclosure may be adapted to meet any program goals. For example, in some embodiments, the program goal may be to maximize single batch yields of reactions with no immediate time limits. In other embodiments, the program goal may be to rebalance biosynthetic yields to produce a specific product, or to produce a particular ratio of products. In other embodiments, the program goal may be to modify the chemical structure of a product, such as lengthening the carbon chain of a polymer. In some embodiments, the program goal may be to improve performance characteristics such as yield, titer, productivity, by-product elimination, tolerance to process excursions, optimal growth temperature and growth rate. In some embodiments, the program goal is improved host performance as measured by volumetric productivity, specific productivity, yield or titre, of a product of interest produced by a microbe.

In other embodiments, the program goal may be to optimize synthesis efficiency of a commercial strain in terms of final product yield per quantity of inputs (e.g., total amount of ethanol produced per pound of sucrose). In other embodiments, the program goal may be to optimize synthesis speed, as measured for example in terms of batch completion rates, or yield rates in continuous culturing systems. In other embodiments, the program goal may be to increase strain resistance to a particular phage, or otherwise increase strain vigor/robustness under culture conditions.

In some embodiments, strain improvement projects may be subject to more than one goal. In some embodiments, the goal of the strain project may hinge on quality, reliability, or overall profitability. In some embodiments, the present disclosure teaches methods of associated selected mutations or groups of mutations with one or more of the strain properties described above.

Persons having ordinary skill in the art will recognize how to tailor strain selection criteria to meet the particular project goal. For example, selections of a strain's single batch max yield at reaction saturation may be appropriate for identifying strains with high single batch yields. Selection based on consistency in yield across a range of temperatures and conditions may be appropriate for identifying strains with increased robustness and reliability.

In some embodiments, the selection criteria for the initial high-throughput phase and the tank-based validation will be identical. In other embodiments, tank-based selection may operate under additional and/or different selection criteria. For example, in some embodiments, high-throughput strain selection might be based on single batch reaction completion yields, while tank-based selection may be expanded to include selections based on yields for reaction speed.

(a) In some embodiments, the selection method involves selecting strains that are resistant to one or more specific metabolites and/or one or more fermentation product of a Saccharopolyspora spp. In some embodiments, a collection of strains which comprise various genetic polymorphs are screened against a given molecule. The collection of strains can be any strain library described in the present disclosure, or combinations thereof. The molecule against which the selection is made can be any final product produced by the strains, or an intermedia product that affects strain growth, or the yield of a final product. For example, in some embodiments, the molecule can be a spinosyn of interest, such as those in Table 4.1 above, or any molecule which affect the production of a spinosyn. Essentially, selection is made for more resistant strains in the presence of one or more predetermined product produced by a. In some embodiments, the method further comprises c) analyzing the performance of the selected strains (e.g., the yield of one or more product produced in the strains) and selecting strains having improved performance compared to the reference microbial strain by HTP screening. In some embodiments, the method further comprises d) identifying position and/or sequences of mutations causing the improved performance. These selected strains with confirmed improved performance form the initial anti-metabolite/fermentation product resistance library. Such a library comprises a plurality of individual microbial strains with unique genetic variations found within each strain of said plurality of individual microbial strains, wherein each of said unique genetic variations corresponds to a single genetic variation selected from the plurality of identifiable genetic variations. In some embodiments, the microbial strains are Saccharopolyspora strains. In some embodiments, the predetermined product produced by the microbial strains is any molecule involved in the spinosyn synthesis pathway, or any molecule that can impact the production of spinosyn. In some embodiments, the predetermined products include, but are not limited to spinosyn A, spinosyn B, spinosyn C, spinosyn D, spinosyn E, spinosyn F, spinosyn G, spinosyn H, spinosyn I, spinosyn J, spinosyn K, spinosyn L, spinosyn M, spinosyn N, spinosyn O, spinosyn P, spinosyn Q, spinosyn R, spinosyn S, spinosyn T, spinosyn U, spinosyn V, spinosyn W, spinosyn X, spinosyn Y, norleucine, norvaline, pseudoaglycones (e.g., PSA, PSD, PSJ, PSL, etc., for the different spinosyn compounds), and/or alpha-Methyl-methionine (aMM).

Sequencing

In some embodiments, the present disclosure teaches whole-genome sequencing of the organisms described herein. In other embodiments, the present disclosure also teaches sequencing of plasmids, PCR products, and other oligos as quality controls to the methods of the present disclosure. Sequencing methods for large and small projects are well known to those in the art.

In some embodiments, any high-throughput technique for sequencing nucleic acids can be used in the methods of the disclosure. In some embodiments, the present disclosure teaches whole genome sequencing. In other embodiments, the present disclosure teaches amplicon sequencing ultra deep sequencing to identify genetic variations. In some embodiments, the present disclosure also teaches novel methods for library preparation, including tagmentation (see WO/2016/073690). DNA sequencing techniques include classic dideoxy sequencing reactions (Sanger method) using labeled terminators or primers and gel separation in slab or capillary; sequencing by synthesis using reversibly terminated labeled nucleotides, pyrosequencing; 454 sequencing; allele specific hybridization to a library of labeled oligonucleotide probes; sequencing by synthesis using allele specific hybridization to a library of labeled clones that is followed by ligation; real time monitoring of the incorporation of labeled nucleotides during a polymerization step; polony sequencing; and SOLiD sequencing.

In one aspect of the disclosure, high-throughput methods of sequencing are employed that comprise a step of spatially isolating individual molecules on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)).

In another embodiment, the methods of the present disclosure comprise amplifying the isolated molecules either before or after they are spatially isolated on a solid surface. Prior amplification may comprise emulsion-based amplification, such as emulsion PCR, or rolling circle amplification. Also taught is Solexa-based sequencing where individual template molecules are spatially isolated on a solid surface, after which they are amplified in parallel by bridge PCR to form separate clonal populations, or clusters, and then sequenced, as described in Bentley et al (cited above) and in manufacturer's instructions (e.g. TruSeq™ Sample Preparation Kit and Data Sheet, Illumina, Inc., San Diego, Calif., 2010); and further in the following references: U.S. Pat. Nos. 6,090,592; 6,300,070; 7,115,400; and EP0972081B1; which are incorporated by reference.

In one embodiment, individual molecules disposed and amplified on a solid surface form clusters in a density of at least 10⁵ clusters per cm²; or in a density of at least 5×10⁵ per cm²; or in a density of at least 10⁶ clusters per cm². In one embodiment, sequencing chemistries are employed having relatively high error rates. In such embodiments, the average quality scores produced by such chemistries are monotonically declining functions of sequence read lengths. In one embodiment, such decline corresponds to 0.5 percent of sequence reads have at least one error in positions 1-75; 1 percent of sequence reads have at least one error in positions 76-100; and 2 percent of sequence reads have at least one error in positions 101-125.

### Computational Analysis and Prediction of Effects of Genome-Wide Genetic Design Criteria

In some embodiments, the present disclosure teaches methods of predicting the effects of particular genetic alterations being incorporated into a given host strain. In further aspects, the disclosure provides methods for generating proposed genetic alterations that should be incorporated into a given host strain, in order for said host to possess a particular phenotypic trait or strain parameter. In given aspects, the disclosure provides predictive models that can be utilized to design novel host strains.

In some embodiments, the present disclosure teaches methods of analyzing the performance results of each round of screening and methods for generating new proposed genome-wide sequence modifications predicted to enhance strain performance in the following round of screening

In some embodiments, the present disclosure teaches that the system generates proposed sequence modifications to host strains based on previous screening results. In some embodiments, the recommendations of the present system are based on the results from the immediately preceding screening. In other embodiments, the recommendations of the present system are based on the cumulative results of one or more of the preceding screenings.

In some embodiments, the recommendations of the present system are based on previously developed HTP genetic design libraries. For example, in some embodiments, the present system is designed to save results from previous screenings, and apply those results to a different project, in the same or different host organisms.

In other embodiments, the recommendations of the present system are based on scientific insights. For example, in some embodiments, the recommendations are based on known properties of genes (from sources such as annotated gene databases and the relevant literature), codon optimization, transcriptional slippage, uORFs, or other hypothesis driven sequence and host optimizations.

In some embodiments, the proposed sequence modifications to a host strain recommended by the system, or predictive model, are carried out by the utilization of one or more of the disclosed molecular tools sets comprising: (1) Promoter swaps, (2) SNP swaps, (3) Start/Stop codon exchanges, (4) Sequence optimization, (5) Stop swaps, and (5) Epistasis mapping.

The HTP genetic engineering platform described herein is agnostic with respect to any particular microbe or phenotypic trait (e.g. production of a particular compound). That is, the platform and methods taught herein can be utilized with any host cell to engineer said host cell to have any desired phenotypic trait. Furthermore, the lessons learned from a given HTP genetic engineering process used to create one novel host cell, can be applied to any number of other host cells, as a result of the storage, characterization, and analysis of a myriad of process parameters that occurs during the taught methods.

As alluded to in the epistatic mapping section, it is possible to estimate the performance (a.k.a. score) of a hypothetical strain obtained by consolidating a collection of mutations from a HTP genetic design library into a particular background via some preferred predictive model. Given such a predictive model, it is possible to score and rank all hypothetical strains accessible to the mutation library via combinatorial consolidation. The below section outlines particular models utilized in the present HTP platform.

Predictive Strain Design

Described herein is an approach for predictive strain design, including: methods of describing genetic changes and strain performance, predicting strain performance based on the composition of changes in the strain, recommending candidate designs with high predicted performance, and filtering predictions to optimize for second-order considerations, e.g. similarity to existing strains, epistasis, or confidence in predictions.

Inputs to Strain Design Model

In one embodiment, for the sake of ease of illustration, input data may comprise two components: (1) sets of genetic changes and (2) relative strain performance. Those skilled in the art will recognize that this model can be readily extended to consider a wide variety of inputs, while keeping in mind the countervailing consideration of overfitting. In addition to genetic changes, some of the input parameters (independent variables) that can be adjusted are cell types (genus, species, strain, phylogenetic characterization, etc.) and process parameters (e.g., environmental conditions, handling equipment, modification techniques, etc.) under which fermentation is conducted with the cells.

The sets of genetic changes can come from the previously discussed collections of genetic perturbations termed HTP genetic design libraries. The relative strain performance can be assessed based upon any given parameter or phenotypic trait of interest (e.g. production of a compound, small molecule, or product of interest).

Cell types can be specified in general categories such as prokaryotic and eukaryotic systems, genus, species, strain, tissue cultures (vs. disperse cells), etc. Process parameters that can be adjusted include temperature, pressure, reactor configuration, and medium composition. Examples of reactor configuration include the volume of the reactor, whether the process is a batch or continuous, and, if continuous, the volumetric flow rate, etc. One can also specify the support structure, if any, on which the cells reside. Examples of medium composition include the concentrations of electrolytes, nutrients, waste products, acids, pH, and the like.

Sets of Genetic Changes from Selected HTP Genetic Design Libraries to be Utilized in the Initial Linear Regression Model that Subsequently is Used to Create the Predictive Strain Design Model

To create a predictive strain design model, genetic changes in strains of the same microbial species are first selected. The history of each genetic change is also provided (e.g., showing the most recent modification in this strain lineage—“last change”). Thus, comparing this strain's performance to the performance of its parent represents a data point concerning the performance of the “last change” mutation.

Built Strain Performance Assessment

The goal of the taught model is to predict strain performance based on the composition of genetic changes introduced to the strain. To construct a standard for comparison, strain performance is computed relative to a common reference strain, by first calculating the median performance per strain, per assay plate. Relative performance is then computed as the difference in average performance between an engineered strain and the common reference strain within the same plate. Restricting the calculations to within-plate comparisons ensures that the samples under consideration all received the same experimental conditions.

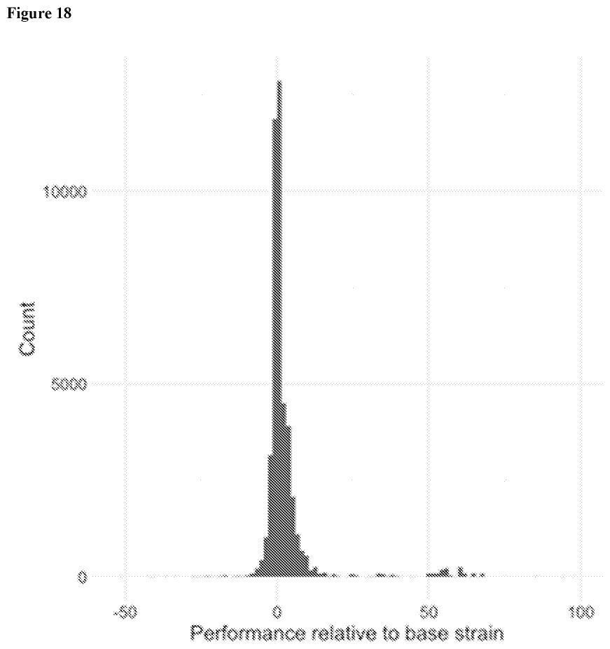

FIG. 18 shows an example in which the distribution of relative strain performances for the input data is under consideration. This was done in Coynebacterium by using the method described in the present disclosure. However, similar procedures have been customized for Saccharopolyspora and are being successfully carried out by the inventors. A relative performance of zero indicates that the engineered strain performed equally well to the in-plate base or “reference” strain. Of interest is the ability of the predictive model to identify the strains that are likely to perform significantly above zero. Further, and more generally, of interest is whether any given strain outperforms its parent by some criteria. In practice, the criteria can be a product titer meeting or exceeding some threshold above the parent level, though having a statistically significant difference from the parent in the desired direction could also be used instead or in addition. The role of the base or “reference” strain is simply to serve as an added normalization factor for making comparisons within or between plates.

A concept to keep in mind is that of differences between: parent strain and reference strain. The parent strain is the background that was used for a current round of mutagenesis. The reference strain is a control strain run in every plate to facilitate comparisons, especially between plates, and is typically the “base strain” as referenced above. But since the base strain (e.g., the wild-type or industrial strain being used to benchmark overall performance) is not necessarily a “base” in the sense of being a mutagenesis target in a given round of strain improvement, a more descriptive term is “reference strain.”

In summary, a base/reference strain is used to benchmark the performance of built strains, generally, while the parent strain is used to benchmark the performance of a specific genetic change in the relevant genetic background.

Ranking the Performance of Built Strains with Linear Regression

The goal of the disclosed model is to rank the performance of built strains, by describing relative strain performance, as a function of the composition of genetic changes introduced into the built strains. As discussed throughout the disclosure, the various HTP genetic design libraries provide the repertoire of possible genetic changes (e.g., genetic perturbations/alterations) that are introduced into the engineered strains. Linear regression is the basis for the currently described exemplary predictive model.

Genetic changes and their effect on relative performance is then input for regression-based modeling. The strain performances are ranked relative to a common base strain, as a function of the composition of the genetic changes contained in the strain.

Linear Regression to Characterize Built Strains

Linear regression is an attractive method for the described HTP genomic engineering platform, because of the ease of implementation and interpretation. The resulting regression coefficients can be interpreted as the average increase or decrease in relative strain performance attributable to the presence of each genetic change.

For example, in some embodiments, this technique allows us to conclude that changing the original promoter to another promoter improves relative strain performance by approximately 1, 2, 3, 4, 5, 6, 7, 8, 9, 10 or more units on average and is thus a potentially highly desirable change, in the absence of any negative epistatic interactions (note: the input is a unit-less normalized value).

The taught method therefore uses linear regression models to describe/characterize and rank built strains, which have various genetic perturbations introduced into their genomes from the various taught libraries.

Predictive Design Modeling

The linear regression model described above, which utilized data from constructed strains, can be used to make performance predictions for strains that haven't yet been built.

The procedure can be summarized as follows: generate in silico all possible configurations of genetic changes→use the regression model to predict relative strain performance→order the candidate strain designs by performance. Thus, by utilizing the regression model to predict the performance of as-yet-unbuilt strains, the method allows for the production of higher performing strains, while simultaneously conducting fewer experiments.

Generate Configurations

When constructing a model to predict performance of as-yet-unbuilt strains, the first step is to produce a sequence of design candidates. This is done by fixing the total number of genetic changes in the strain, and then defining all possible combinations of genetic changes. For example, one can set the total number of potential genetic changes/perturbations to 29 (e.g. 29 possible SNPs, or 29 different promoters, or any combination thereof as long as the universe of genetic perturbations is 29) and then decide to design all possible 3-member combinations of the 29 potential genetic changes, which will result in 3,654 candidate strain designs.

To provide context to the aforementioned 3,654 candidate strains, consider that one can calculate the number of non-redundant groupings of size r from n possible members using n!/((n−r)!*r!). If r=3, n=29 gives 3,654. Thus, if one designs all possible 3-member combinations of 29 potential changes the results is 3,654 candidate strains.

Predict Performance of New Strain Designs

Using the linear regression constructed above with the combinatorial configurations as input, one can then predict the expected relative performance of each candidate design. For example, the composition of changes for the top 100 predicted strain designs can be summarized in a 2-dimensional map, in which the x-axis lists the pool of potential genetic changes (29 possible genetic changes), and the y-axis shows the rank order. Black cells can be used to indicate the presence of a particular change in the candidate design, while white cells can be used to indicate the absence of that change.

Predictive accuracy should increase over time as new observations are used to iteratively retrain and refit the model. Results from a study by the inventors illustrate the methods by which the predictive model can be iteratively retrained and improved. The quality of model predictions can be assessed through several methods, including a correlation coefficient indicating the strength of association between the predicted and observed values, or the root-mean-square error, which is a measure of the average model error. Using a chosen metric for model evaluation, the system may define rules for when the model should be retrained.

A couple of unstated assumptions to the above model include: (1) there are no epistatic interactions; and (2) the genetic changes/perturbations utilized to build the predictive model were all made in the same background, as the proposed combinations of genetic changes.

Filtering for Second-Order Features

The above illustrative example focused on linear regression predictions based on predicted host cell performance. In some embodiments, the present linear regression methods can also be applied to non-biomolecule factors, such as saturation biomass, resistance, or other measurable host cell features. Thus the methods of the present disclosure also teach in considering other features outside of predicted performance when prioritizing the candidates to build. Assuming there is additional relevant data, nonlinear terms are also included in the regression model.

Closeness with Existing Strains

Predicted strains that are similar to ones that have already been built could result in time and cost savings despite not being a top predicted candidate

Diversity of Changes

When constructing the aforementioned models, one cannot be certain that genetic changes will truly be additive (as assumed by linear regression and mentioned as an assumption above) due to the presence of epistatic interactions. Therefore, knowledge of genetic change dissimilarity can be used to increase the likelihood of positive additivity. If one knows, for example, that the changes from the top ranked strain are on the same metabolic pathway and have similar performance characteristics, then that information could be used to select another top ranking strain with a dissimilar composition of changes. As described in the section above concerning epistasis mapping, the predicted best genetic changes may be filtered to restrict selection to mutations with sufficiently dissimilar response profiles. Alternatively, the linear regression may be a weighted least squares regression using the similarity matrix to weight predictions.

Diversity of Predicted Performance

Finally, one may choose to design strains with middling or poor predicted performance, in order to validate and subsequently improve the predictive models.

Iterative Strain Design Optimization

In embodiments, the order placement engine 208 places a factory order to the factory 210 to manufacture microbial strains incorporating the top candidate mutations. In feedback-loop fashion, the results may be analyzed by the analysis equipment 214 to determine which microbes exhibit desired phenotypic properties (314). During the analysis phase, the modified strain cultures are evaluated to determine their performance, i.e., their expression of desired phenotypic properties, including the ability to be produced at industrial scale. For example, the analysis phase uses, among other things, image data of plates to measure microbial colony growth as an indicator of colony health. The analysis equipment 214 is used to correlate genetic changes with phenotypic performance, and save the resulting genotype-phenotype correlation data in libraries, which may be stored in library 206, to inform future microbial production.

In particular, the candidate changes that actually result in sufficiently high measured performance may be added as rows in the database to tables such as Table 4 above. In this manner, the best performing mutations are added to the predictive strain design model in a supervised machine learning fashion.

LIMS iterates the design/build/test/analyze cycle based on the correlations developed from previous factory runs. During a subsequent cycle, the analysis equipment 214 alone, or in conjunction with human operators, may select the best candidates as base strains for input back into input interface 202, using the correlation data to fine tune genetic modifications to achieve better phenotypic performance with finer granularity. In this manner, the laboratory information management system of embodiments of the disclosure implements a quality improvement feedback loop.

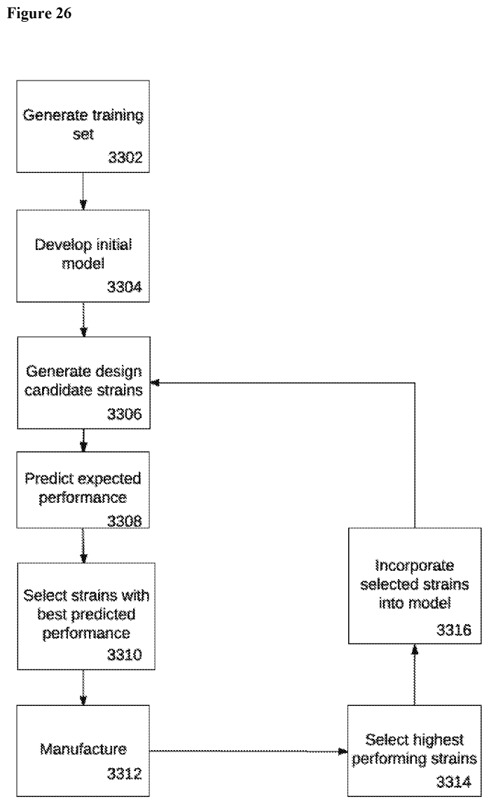

In sum, with reference to the flowchart of FIG. 26 the iterative predictive strain design workflow may be described as follows:
Generate a training set of input and output variables, e.g., genetic changes as inputs and performance features as outputs (3302). Generation may be performed by the analysis equipment 214 based upon previous genetic changes and the corresponding measured performance of the microbial strains incorporating those genetic changes.
Develop an initial model (e.g., linear regression model) based upon training set (3304). This may be performed by the analysis equipment 214.
Generate design candidate strains (3306)
In one embodiment, the analysis equipment 214 may fix the number of genetic changes to be made to a background strain, in the form of combinations of changes. To represent these changes, the analysis equipment 214 may provide to the interpreter 204 one or more DNA specification expressions representing those combinations of changes. (These genetic changes or the microbial strains incorporating those changes may be referred to as “test inputs.”) The interpreter 204 interprets the one or more DNA specifications, and the execution engine 207 executes the DNA specifications to populate the DNA specification with resolved outputs representing the individual candidate design strains for those changes.
Based upon the model, the analysis equipment 214 predicts expected performance of each candidate design strain (3308).
The analysis equipment 214 selects a limited number of candidate designs, e.g., 100, with highest predicted performance (3310).
As described elsewhere herein with respect to epistasis mapping, the analysis equipment 214 may account for second-order effects such as epistasis, by, e.g., filtering top designs for epistatic effects, or factoring epistasis into the predictive model.
Build the filtered candidate strains (at the factory 210) based on the factory order generated by the order placement engine 208 (3312).
The analysis equipment 214 measures the actual performance of the selected strains, selects a limited number of those selected strains based upon their superior actual performance (3314), and adds the design changes and their resulting performance to the predictive model (3316). In the linear regression example, add the sets of design changes and their associated performance as new rows in Table 4.
The analysis equipment 214 then iterates back to generation of new design candidate strains (3306), and continues iterating until a stop condition is satisfied. The stop condition may comprise, for example, the measured performance of at least one microbial strain satisfying a performance metric, such as yield, growth rate, or titer.

In the example above, the iterative optimization of strain design employs feedback and linear regression to implement machine learning. In general, machine learning may be described as the optimization of performance criteria, e.g., parameters, techniques or other features, in the performance of an informational task (such as classification or regression) using a limited number of examples of labeled data, and then performing the same task on unknown data. In supervised machine learning such as that of the linear regression example above, the machine (e.g., a computing device) learns, for example, by identifying patterns, categories, statistical relationships, or other attributes, exhibited by training data. The result of the learning is then used to predict whether new data will exhibit the same patterns, categories, statistical relationships or other attributes.

Embodiments of the disclosure may employ other supervised machine learning techniques when training data is available. In the absence of training data, embodiments may employ unsupervised machine learning. Alternatively, embodiments may employ semi-supervised machine learning, using a small amount of labeled data and a large amount of unlabeled data. Embodiments may also employ feature selection to select the subset of the most relevant features to optimize performance of the machine learning model. Depending upon the type of machine learning approach selected, as alternatives or in addition to linear regression, embodiments may employ for example, logistic regression, neural networks, support vector machines (SVMs), decision trees, hidden Markov models, Bayesian networks, Gram Schmidt, reinforcement-based learning, cluster-based learning including hierarchical clustering, genetic algorithms, and any other suitable learning machines known in the art. In particular, embodiments may employ logistic regression to provide probabilities of classification (e.g., classification of genes into different functional groups) along with the classifications themselves. See, e.g., Shevade, A simple and efficient algorithm for gene selection using sparse logistic regression, Bioinformatics, Vol. 19, No. 17 2003, pp. 2246-2253, Leng, et al., Classification using functional data analysis for temporal gene expression data, Bioinformatics, Vol. 22, No. 1, Oxford University Press (2006), pp. 68-76, all of which are incorporated by reference in their entirety herein.

Embodiments may employ graphics processing unit (GPU) accelerated architectures that have found increasing popularity in performing machine learning tasks, particularly in the form known as deep neural networks (DNN). Embodiments of the disclosure may employ GPU-based machine learning, such as that described in GPU-Based Deep Learning Inference: A Performance and Power Analysis, NVidia Whitepaper, November 2015, Dahl, et al., Multi-task Neural Networks for QSAR Predictions, Dept. of Computer Science, Univ. of Toronto, June 2014 (arXiv:1406.1231 [stat.ML]), all of which are incorporated by reference in their entirety herein. Machine learning techniques applicable to embodiments of the disclosure may also be found in, among other references, Libbrecht, et al., Machine learning applications in genetics and genomics, Nature Reviews: Genetics, Vol. 16, June 2015, Kashyap, et al., Big Data Analytics in Bioinformatics: A Machine Learning Perspective, Journal of Latex Class Files, Vol. 13, No. 9, September 2014, Prompramote, et al., Machine Learning in Bioinformatics, Chapter 5 of Bioinformatics Technologies, pp. 117-153, Springer Berlin Heidelberg 2005, all of which are incorporated by reference in their entirety herein.

### Iterative Predictive Strain Design: Example

The following provides an example application of the iterative predictive strain design workflow outlined above.

An initial set of training inputs and output variables was prepared. This set comprised 1864 unique engineered strains with defined genetic composition. Each strain contained between 5 and 15 engineered changes. A total of 336 unique genetic changes were present in the training.

An initial predictive computer model was developed. The implementation used a generalized linear model (Kernel Ridge Regression with 4th order polynomial kernel). The implementation models two distinct phenotypes (yield and productivity). These phenotypes were combined as weighted sum to obtain a single score for ranking, as shown below. Various model parameters, e.g. regularization factor, were tuned via k-fold cross validation over the designated training data.

The implementation does not incorporate any explicit analysis of interaction effects as described in the Epistasis Mapping section above. However, as those skilled in the art would understand, the implemented generalized linear model may capture interaction effects implicitly through the second, third and fourth order terms of the kernel.

The model is trained against the training set. After training, a significant quality fitting of the yield model to the training data can be demonstrated.

Candidate strains are then generated. This embodiments includes a serial build constraint associated with the introduction of new genetic changes to a parent strain. Here, candidates are not considered simply as a function of the desired number of changes. Instead, the analysis equipment 214 selects, as a starting point, a collection of previously designed strains known to have high performance metrics (“seed strains”). The analysis equipment 214 individually applies genetic changes to each of the seed strains. The introduced genetic changes do not include those already present in the seed strain. For various technical, biological or other reasons, certain mutations are explicitly required, or explicitly excluded

Based upon the model, the analysis equipment 214 predicted the performance of candidate strain designs. The analysis equipment 214 ranks candidates from “best” to “worst” based on predicted performance with respect to two phenotypes of interest (yield and productivity). Specifically, the analysis equipment 214 uses a weighted sum to score a candidate strain:

Score=0.8*yield/max(yields)+0.2*prod/max(prods),

where yield represents predicted yield for the candidate strain,
max(yields) represents the maximum yield over all candidate strains,
prod represents productivity for the candidate strain, and
max(prods) represents the maximum yield over all candidate strains.

The analysis equipment 214 generates a final set of recommendations from the ranked list of candidates by imposing both capacity constraints and operational constraints. In some embodiments, the capacity limit can be set at a given number, such as 48 computer-generated candidate design strains.

The trained model (described above) can be used to predict the expected performance (for yield and productivity) of each candidate strain. The analysis equipment 214 can rank the candidate strains using the scoring function given above. Capacity and operational constraints can be then applied to yield a filtered set of 48 candidate strains. Filtered candidate strains are then built (at the factory 210) based on a factory order generated by the order placement engine 208 (3312). The order can be based upon DNA specifications corresponding to the candidate strains.

In practice, the build process has an expected failure rate whereby a random set of strains is not built.

The analysis equipment 214 can also be used to measure the actual yield and productivity performance of the selected strains. The analysis equipment 214 can evaluate the model and recommended strains based on three criteria: model accuracy; improvement in strain performance; and equivalence (or improvement) to human expert-generated designs.

The yield and productivity phenotypes can be measured for recommended strains and compared to the values predicted by the model.

Next, the analysis equipment 214 computes percentage performance change from the parent strain for each of the recommended strains.

Predictive accuracy can be assessed through several methods, including a correlation coefficient indicating the strength of association between the predicted and observed values, or the root-mean-square error, which is a measure of the average model error. Over many rounds of experimentation, model predictions may drift, and new genetic changes may be added to the training inputs to improve predictive accuracy. For this example, design changes and their resulting performance were added to the predictive model (3316).

### Genomic Design and Engineering as a Service

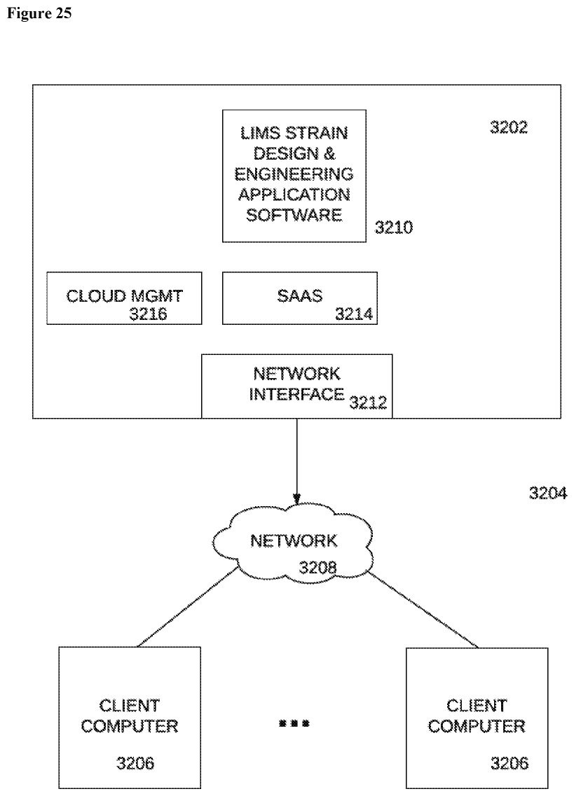

In embodiments of the disclosure, the LIMS system software 3210 of FIG. 25 may be implemented in a cloud computing system 3202 of FIG. 25, to enable multiple users to design and build microbial strains according to embodiments of the present disclosure. FIG. 25 illustrates a cloud computing environment 3204 according to embodiments of the present disclosure. Client computers 3206, such as those illustrated in FIG. 25, access the LIMS system via a network 3208, such as the Internet. In embodiments, the LIMS system application software 3210 resides in the cloud computing system 3202. The LIMS system may employ one or more computing systems using one or more processors, of the type illustrated in FIG. 25. The cloud computing system itself includes a network interface 3212 to interface the LIMS system applications 3210 to the client computers 3206 via the network 3208. The network interface 3212 may include an application programming interface (API) to enable client applications at the client computers 3206 to access the LIMS system software 3210. In particular, through the API, client computers 3206 may access components of the LIMS system 200, including without limitation the software running the input interface 202, the interpreter 204, the execution engine 207, the order placement engine 208, the factory 210, as well as test equipment 212 and analysis equipment 214. A software as a service (SaaS) software module 3214 offers the LIMS system software 3210 as a service to the client computers 3206. A cloud management module 3216 manages access to the LIMS system 3210 by the client computers 3206. The cloud management module 3216 may enable a cloud architecture that employs multitenant applications, virtualization or other architectures known in the art to serve multiple users.

### Genomic Automation

Automation of the methods of the present disclosure enables high-throughput phenotypic screening and identification of target products from multiple test strain variants simultaneously.

The aforementioned genomic engineering predictive modeling platform is premised upon the fact that hundreds and thousands of mutant strains are constructed in a high-throughput fashion. The robotic and computer systems described below are the structural mechanisms by which such a high-throughput process can be carried out.

In some embodiments, the present disclosure teaches methods of improving host cell productivities, or rehabilitating industrial strains. As part of this process, the present disclosure teaches methods of assembling DNA, building new strains, screening cultures in plates, and screening cultures in models for tank fermentation. In some embodiments, the present disclosure teaches that one or more of the aforementioned methods of creating and testing new host strains is aided by automated robotics.

In some embodiments, the present disclosure teaches a high-throughput strain engineering platform as depicted in FIG. 6A-B.

HTP Robotic Systems

In some embodiments, the automated methods of the disclosure comprise a robotic system. The systems outlined herein are generally directed to the use of 96- or 384-well microtiter plates, but as will be appreciated by those in the art, any number of different plates or configurations may be used. In addition, any or all of the steps outlined herein may be automated; thus, for example, the systems may be completely or partially automated.

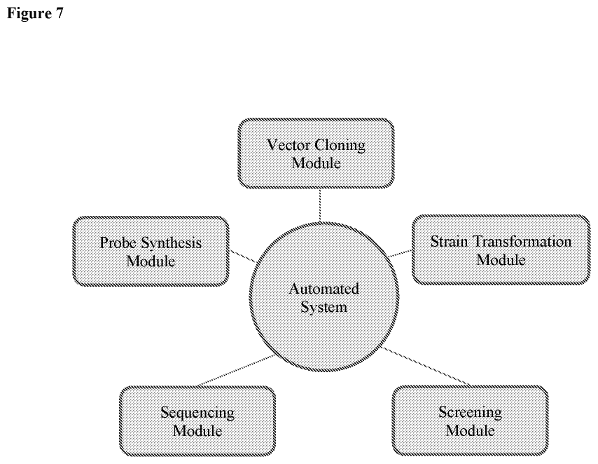

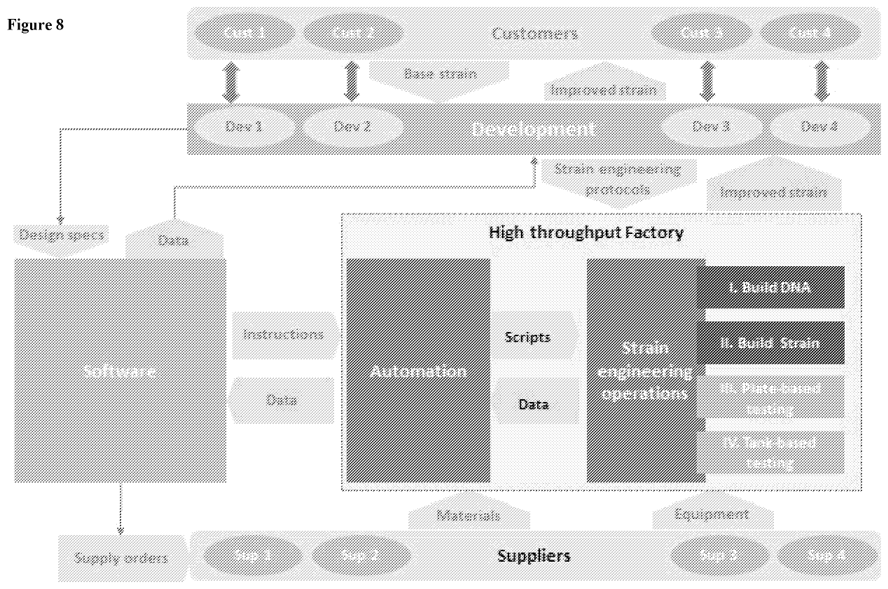

In some embodiments, the automated systems of the present disclosure comprise one or more work modules. For example, in some embodiments, the automated system of the present disclosure comprises a DNA synthesis module, a vector cloning module, a strain transformation module, a screening module, and a sequencing module (see FIG. 7).

As will be appreciated by those in the art, an automated system can include a wide variety of components, including, but not limited to: liquid handlers; one or more robotic arms; plate handlers for the positioning of microplates; plate sealers, plate piercers, automated lid handlers to remove and replace lids for wells on non-cross contamination plates; disposable tip assemblies for sample distribution with disposable tips; washable tip assemblies for sample distribution; 96 well loading blocks; integrated thermal cyclers; cooled reagent racks; microtiter plate pipette positions (optionally cooled); stacking towers for plates and tips; magnetic bead processing stations; filtrations systems; plate shakers; barcode readers and applicators; and computer systems.

In some embodiments, the robotic systems of the present disclosure include automated liquid and particle handling enabling high-throughput pipetting to perform all the steps in the process of gene targeting and recombination applications. This includes liquid and particle manipulations such as aspiration, dispensing, mixing, diluting, washing, accurate volumetric transfers; retrieving and discarding of pipette tips; and repetitive pipetting of identical volumes for multiple deliveries from a single sample aspiration. These manipulations are cross-contamination-free liquid, particle, cell, and organism transfers. The instruments perform automated replication of microplate samples to filters, membranes, and/or daughter plates, high-density transfers, full-plate serial dilutions, and high capacity operation.

In some embodiments, the customized automated liquid handling system of the disclosure is a TECAN machine (e.g. a customized TECAN Freedom Evo).

In some embodiments, the automated systems of the present disclosure are compatible with platforms for multi-well plates, deep-well plates, square well plates, reagent troughs, test tubes, mini tubes, microfuge tubes, cryovials, filters, micro array chips, optic fibers, beads, agarose and acrylamide gels, and other solid-phase matrices or platforms are accommodated on an upgradeable modular deck. In some embodiments, the automated systems of the present disclosure contain at least one modular deck for multi-position work surfaces for placing source and output samples, reagents, sample and reagent dilution, assay plates, sample and reagent reservoirs, pipette tips, and an active tip-washing station.

In some embodiments, the automated systems of the present disclosure include high-throughput electroporation systems. In some embodiments, the high-throughput electroporation systems are capable of transforming cells in 96 or 384-well plates. In some embodiments, the high-throughput electroporation systems include VWR® High-throughput Electroporation Systems, BTX™, Bio-Rad® Gene Pulser MXcell™ or other multi-well electroporation system.

In some embodiments, the integrated thermal cycler and/or thermal regulators are used for stabilizing the temperature of heat exchangers such as controlled blocks or platforms to provide accurate temperature control of incubating samples from 0° C. to 100° C.

In some embodiments, the automated systems of the present disclosure are compatible with interchangeable machine-heads (single or multi-channel) with single or multiple magnetic probes, affinity probes, replicators or pipetters, capable of robotically manipulating liquid, particles, cells, and multi-cellular organisms. Multi-well or multi-tube magnetic separators and filtration stations manipulate liquid, particles, cells, and organisms in single or multiple sample formats.

In some embodiments, the automated systems of the present disclosure are compatible with camera vision and/or spectrometer systems. Thus, in some embodiments, the automated systems of the present disclosure are capable of detecting and logging color and absorption changes in ongoing cellular cultures.

In some embodiments, the automated system of the present disclosure is designed to be flexible and adaptable with multiple hardware add-ons to allow the system to carry out multiple applications. The software program modules allow creation, modification, and running of methods. The system's diagnostic modules allow setup, instrument alignment, and motor operations. The customized tools, labware, and liquid and particle transfer patterns allow different applications to be programmed and performed. The database allows method and parameter storage. Robotic and computer interfaces allow communication between instruments.

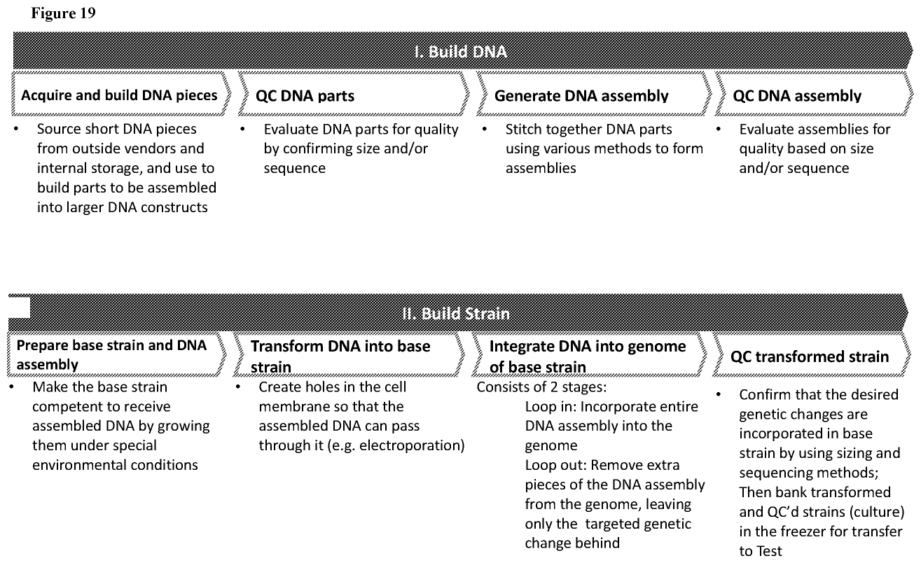

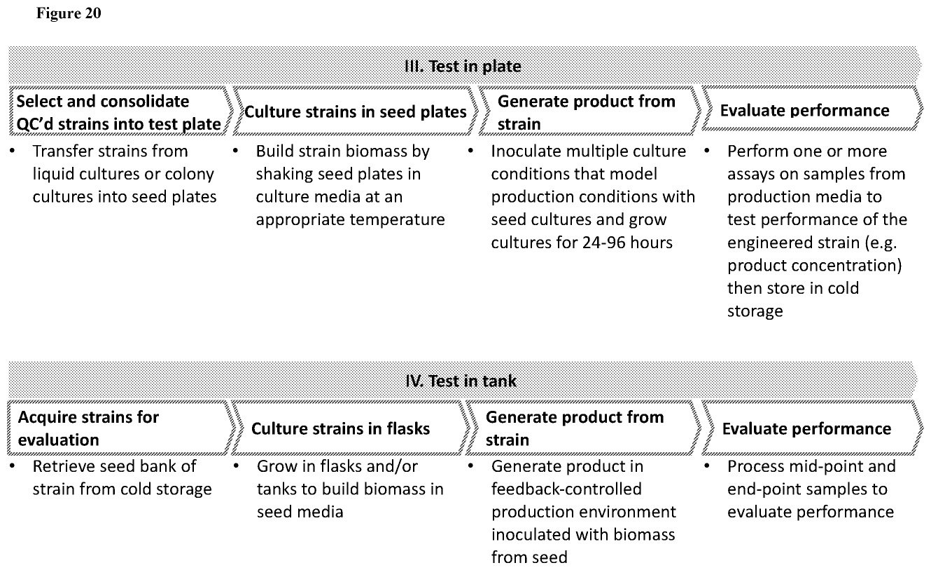

Thus, in some embodiments, the present disclosure teaches a high-throughput strain engineering platform, as depicted in FIG. 19.

Persons having skill in the art will recognize the various robotic platforms capable of carrying out the HTP engineering methods of the present disclosure. Table 5 below provides a non-exclusive list of scientific equipment capable of carrying out each step of the HTP engineering steps of the present disclosure as described in FIG. 19.

TABLE 5
Non-exclusive list of Scientific Equipment Compatible with the HTP engineering
methods of the present disclosure.

| | Equipment | | Compatible Equipment |
|---|---|---|---|
| | Type | Operation(s) performed | Make/Model/Configuration |
| Acquire and build DNA pieces | liquid handlers | Hitpicking (combining by | Hamilton Microlab STAR, |
| | | transferring) | Labcyte Echo 550, Tecan |
| | | primers/templates for | EVO 200, Beckman Coulter |
| | | PCR amplification of | Biomek FX, or equivalents |
| | | DNA parts | |
| | Thermal | PCR amplification of | Inheco Cycler, ABI 2720, ABI |
| | cyclers | DNA parts | Proflex 384, ABI Veriti, or |
| | | | equivalents |
| QC DNA parts | Fragment | gel electrophoresis to | Agilent Bioanalyzer, AATI |
| | analyzers | confirm PCR products of | Fragment Analyzer, or |
| | (capillary | appropriate size | equivalents |
| | electrophoresis) | | |
| | Sequencer | Verifying sequence of | Beckman Ceq-8000, Beckman |
| | (sanger: | parts/templates | GenomeLab ™, or equivalents |
| | Beckman) | | |
| | NGS (next | Verifying sequence of | Illumina MiSeq series |
| | generation | parts/templates | sequences, illumina Hi-Seq, |
| | sequencing) | | Ion torrent, pac bio or other |
| | instrument | | equivalents |
| | nanodrop/plate | assessing concentration | Molecular Devices |
| | reader | of DNA samples | SpectraMax M5, Tecan |
| | | | M1000, or equivalents. |
| Generate DNA assembly | liquid handlers | Hitpicking (combining by | Hamilton Microlab STAR, |
| | | transferring) DNA parts | Labcyte Echo 550, Tecan |
| | | for assembly along with | EVO 200, Beckman Coulter |
| | | cloning vector, addition | Biomek FX, or equivalents |
| | | of reagents for assembly | |
| | | reaction/process | |
| QC DNA assembly | Colony pickers | for inoculating colonies | Scirobotics Pickolo, |
| | | in liquid media | Molecular Devices QPix 420 |
| | liquid handlers | Hitpicking | Hamilton Microlab STAR, |
| | | primers/templates, | Labcyte Echo 550, Tecan |
| | | diluting samples | EVO 200, Beckman Coulter |
| | | | Biomek FX, or equivalents |
| | Fragment | gel electrophoresis to | Agilent Bioanalyzer, AATI |
| | analyzers | confirm assembled | Fragment Analyzer |
| | (capillary | products of appropriate | |
| | electrophoresis) | size | |
| | Sequencer | Verifying sequence of | ABI3730 Thermo Fisher, |
| | (sanger: | assembled plasmids | Beckman Ceq-8000, Beckman |
| | Beckman) | | GenomeLab ™, or equivalents |
| | NGS (next | Verifying sequence of | Illumina MiSeq series |
| | generation | assembled plasmids | sequences, illumina Hi-Seq, |
| | sequencing) | | Ion torrent, pac bio or other |
| | instrument | | equivalents |
| Prepare base strain and DNA | centrifuge | spinning/pelleting cells | Beckman Avanti floor |
| assembly | | | centrifuge, |
| | | | Hettich Centrifuge |
| Transform DNA into base strain | Electroporators | electroporative | BTX Gemini X2, BIO-RAD |
| | | transformation of cells | MicroPulser Electroporator |
| | Ballistic | ballistic transformation of | BIO-RAD PDS1000 |
| | transformation | cells | |
| | Incubators, | for chemical | Inheco Cycler, ABI 2720, ABI |
| | thermal cyclers | transformation/heat | Proflex 384, ABI Veriti, or |
| | | shock | equivalents |
| | Liquid handlers | for combining DNA, | Hamilton Microlab STAR, |
| | | cells, buffer | Labcyte Echo 550, Tecan |
| | | | EVO 200, Beckman Coulter |
| | | | Biomek FX, or equivalents |
| Integrate DNA into | Colony pickers | for inoculating colonies | Scirobotics Pickolo, |
| genome of base strain | | in liquid media | Molecular Devices QPix 420 |
| | Liquid handlers | For transferring cells | Hamilton Microlab STAR, |
| | | onto Agar, transferring | Labcyte Echo 550, Tecan |
| | | from culture plates to | EVO 200, Beckman Coulter |
| | | different culture plates | Biomek FX, or equivalents |
| | | (inoculation into other | |
| | | selective media) | |
| | Platform | incubation with shaking | Kuhner Shaker ISF4-X, |
| | shaker- | of microtiter plate | Infors-ht Multitron Pro |
| | incubators | cultures | |
| QC transformed strain | Colony pickers | for inoculating colonies | Scirobotics Pickolo, |
| | | in liquid media | Molecular Devices QPix 420 |
| | liquid handlers | Hitpicking | Hamilton Microlab STAR, |
| | | primers/templates, | Labcyte Echo 550, Tecan |
| | | diluting samples | EVO 200, Beckman Coulter |
| | | | Biomek FX, or equivalents |
| | Thermal | cPCR verification of | Inheco Cycler, ABI 2720, ABI |
| | cyclers | strains | Proflex 384, ABI Veriti, or |
| | | | equivalents |
| | Fragment | gel electrophoresis to | Infors-ht Multitron Pro, |
| | analyzers | confirm cPCR products | Kuhner ShakerISF4-X |
| | (capillary | of appropriate size | |
| | electrophoresis) | | |
| | Sequencer | Sequence verification of | Beckman Ceq-8000, Beckman |
| | (sanger: | introduced modification | GenomeLab ™, or equivalents |
| | Beckman) | | |
| | NGS (next | Sequence verification of | Illumina MiSeq series |
| | generation | introduced modification | sequences, illumina Hi-Seq, |
| | sequencing) | | Ion torrent, pac bio or other |
| | instrument | | equivalents |
| Select and consolidate QC'd strains into test | Liquid handlers | For transferring from | Hamilton Microlab STAR, |
| plate | | culture plates to different | Labcyte Echo 550, Tecan |
| | | culture plates | EVO 200, Beckman Coulter |
| | | (inoculation into | Biomek FX, or equivalents |
| | | production media) | |
| | Colony pickers | for inoculating colonies | Scirobotics Pickolo, |
| | | in liquid media | Molecular Devices QPix 420 |
| | Platform | incubation with shaking | Kuhner Shaker ISF4-X, |
| | shaker- | of microtiter plate | Infors-ht Multitron Pro |
| | incubators | cultures | |
| Culture strains in seed plates | Liquid handlers | For transferring from | Hamilton Microlab STAR, |
| | | culture plates to different | Labcyte Echo 550, Tecan |
| | | culture plates | EVO 200, Beckman Coulter |
| | | (inoculation into | Biomek FX, or equivalents |
| | | production media) | |
| | Platform | incubation with shaking | Kuhner Shaker ISF4-X, |
| | shaker- | of microtiter plate | Infors-ht Multitron Pro |
| | incubators | cultures | |
| | liquid | Dispense liquid culture | Well mate (Thermo), |
| | dispensers | media into microtiter | Benchcel2R (velocity 11), |
| | | plates | plateloc (velocity 11) |
| | microplate | apply barcoders to plates | Microplate labeler (a2+ cab- |
| | labeler | | agilent), benchcell 6R |
| | | | (velocity 11) |
| Generate product from strain | Liquid handlers | For transferring from | Hamilton Microlab STAR, |
| | | culture plates to different | Labcyte Echo 550, Tecan |
| | | culture plates | EVO 200, Beckman Coulter |
| | | (inoculation into | Biomek FX, or equivalents |
| | | production media) | |
| | Platform | incubation with shaking | Kuhner Shaker ISF4-X, |
| | shaker- | of microtiter plate | Infors-ht Multitron Pro |
| | incubators | cultures | |
| | liquid | Dispense liquid culture | well mate (Thermo), |
| | dispensers | media into multiple | Benchcel2R (velocity 11), |
| | | microtiter plates and seal | plateloc (velocity 11) |
| | | plates | |
| | microplate | Apply barcodes to plates | microplate labeler (a2+ cab- |
| | labeler | | agilent), benchcell 6R |
| | | | (velocity 11) |
| Evaluate performance | Liquid handlers | For processing culture | Hamilton Microlab STAR, |
| | | broth for downstream | Labcyte Echo 550, Tecan |
| | | analytical | EVO 200, Beckman Coulter |
| | | | Biomek FX, or equivalents |
| | UHPLC, HPLC | quantitative analysis of | Agilent 1290 Series UHPLC |
| | | precursor and target | and 1200 Series HPLC with |
| | | compounds | UV and RI detectors, or |
| | | | equivalent; also any LC/MS |
| | LC/MS | highly specific analysis | Agilent 6490 QQQ and 6550 |
| | | of precursor and target | QTOF coupled to 1290 Series |
| | | compounds as well as | UHPLC |
| | | side and degradation | |
| | | products | |
| | Spectrophotometer | Quantification of | Tecan M1000, spectramax |
| | | different compounds | M5, Genesys 10S |
| | | using spectrophotometer | |
| | | based assays | |
| Culture strains in | : | incubation with shaking | Sartorius, DASGIPs |
| flasks | | | (Eppendorf), BIO-FLOs |
| | | | (Sartorius-stedim). Applikon |
| | Platform | | innova 4900, or any |
| | shakers | | equivalent |
| Generate product | Fermenters: DASGIPs (Eppendorf), BIO-FLOs (Sartorius-stedim) | | |
| from strain | | | |
| Evaluate performance | Liquid handlers | For transferring from | Hamilton Microlab STAR, |
| | | culture plates to different | Labcyte Echo 550, Tecan |
| | | culture plates | EVO 200, Beckman Coulter |
| | | (inoculation into | Biomek FX, or equivalents |
| | | production media) | |
| | UHPLC, HPLC | quantitative analysis of | Agilent 1290 Series UHPLC |
| | | precursor and target | and 1200 Series HPLC with |
| | | compounds | UV and RI detectors, or |
| | | | equivalent; also any LC/MS |
| | LC/MS | highly specific analysis | Agilent 6490 QQQ and 6550 |
| | | of precursor and target | QTOF coupled to 1290 Series |
| | | compounds as well as | UHPLC |
| | | side and degradation | |
| | | products | |
| | Flow cytometer | Characterize strain | BD Accuri, Millipore Guava |
| | | performance (measure | |
| | | viability) | |
| | Spectrophotometer | Characterize strain | Tecan M1000, Spectramax |
| | | performance (measure | M5, or other equivalents |
| | | biomass) | |

Computer System Hardware

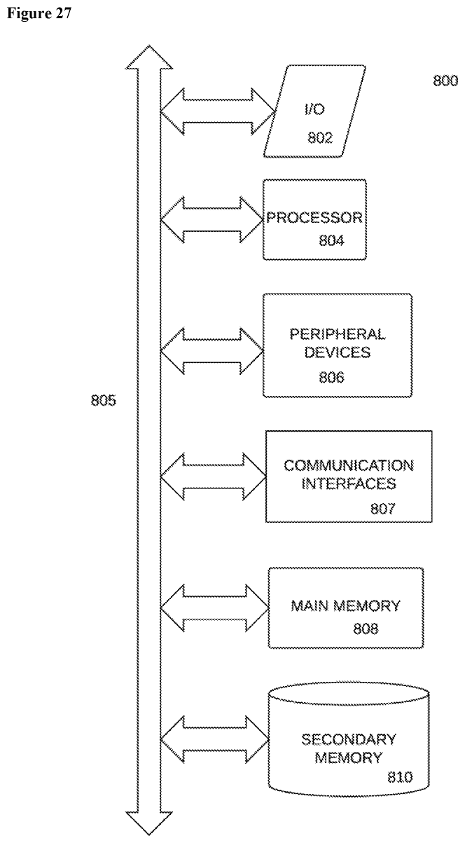

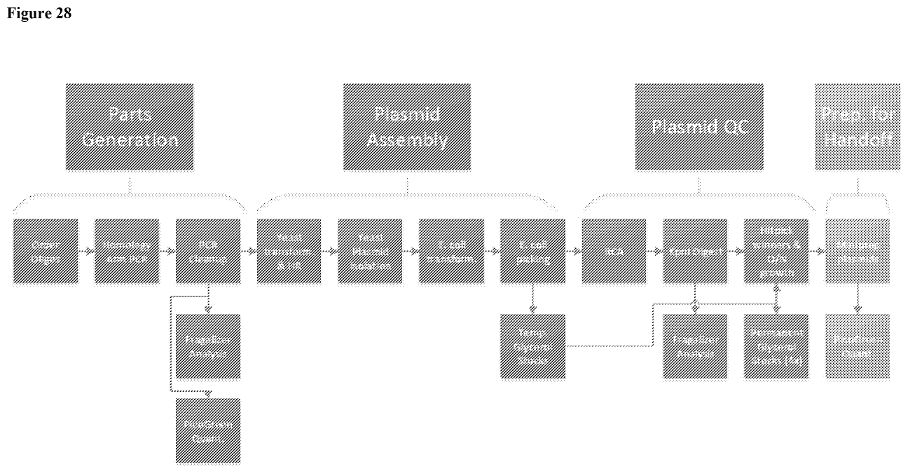

FIG. 27 illustrates an example of a computer system 800 that may be used to execute program code stored in a non-transitory computer readable medium (e.g., memory) in accordance with embodiments of the disclosure. The computer system includes an input/output subsystem 802, which may be used to interface with human users and/or other computer systems depending upon the application. The I/O subsystem 802 may include, e.g., a keyboard, mouse, graphical user interface, touchscreen, or other interfaces for input, and, e.g., an LED or other flat screen display, or other interfaces for output, including application program interfaces (APIs). Other elements of embodiments of the disclosure, such as the components of the LIMS system, may be implemented with a computer system like that of computer system 800.

Program code may be stored in non-transitory media such as persistent storage in secondary memory 810 or main memory 808 or both. Main memory 808 may include volatile memory such as random access memory (RAM) or non-volatile memory such as read only memory (ROM), as well as different levels of cache memory for faster access to instructions and data. Secondary memory may include persistent storage such as solid state drives, hard disk drives or optical disks. One or more processors 804 reads program code from one or more non-transitory media and executes the code to enable the computer system to accomplish the methods performed by the embodiments herein. Those skilled in the art will understand that the processor(s) may ingest source code, and interpret or compile the source code into machine code that is understandable at the hardware gate level of the processor(s) 804. The processor(s) 804 may include graphics processing units (GPUs) for handling computationally intensive tasks. Particularly in machine learning, one or more CPUs 804 may offload the processing of large quantities of data to one or more GPUs 804.

The processor(s) 804 may communicate with external networks via one or more communications interfaces 807, such as a network interface card, WiFi transceiver, etc. A bus 805 communicatively couples the I/O subsystem 802, the processor(s) 804, peripheral devices 806, communications interfaces 807, memory 808, and persistent storage 810. Embodiments of the disclosure are not limited to this representative architecture. Alternative embodiments may employ different arrangements and types of components, e.g., separate buses for input-output components and memory subsystems.

Those skilled in the art will understand that some or all of the elements of embodiments of the disclosure, and their accompanying operations, may be implemented wholly or partially by one or more computer systems including one or more processors and one or more memory systems like those of computer system 800. In particular, the elements of the LIMS system 200 and any robotics and other automated systems or devices described herein may be computer-implemented. Some elements and functionality may be implemented locally and others may be implemented in a distributed fashion over a network through different servers, e.g., in client-server fashion, for example. In particular, server-side operations may be made available to multiple clients in a software as a service (SaaS) fashion, as shown in FIG. 25.

The term component in this context refers broadly to software, hardware, or firmware (or any combination thereof) component. Components are typically functional components that can generate useful data or other output using specified input(s). A component may or may not be self-contained. An application program (also called an “application”) may include one or more components, or a component can include one or more application programs.

Some embodiments include some, all, or none of the components along with other modules or application components. Still yet, various embodiments may incorporate two or more of these components into a single module and/or associate a portion of the functionality of one or more of these components with a different component.

The term “memory” can be any device or mechanism used for storing information. In accordance with some embodiments of the present disclosure, memory is intended to encompass any type of, but is not limited to: volatile memory, nonvolatile memory, and dynamic memory. For example, memory can be random access memory, memory storage devices, optical memory devices, magnetic media, floppy disks, magnetic tapes, hard drives, SIMMs, SDRAM, DIMMs, RDRAM, DDR RAM, SODIMMS, erasable programmable read-only memories (EPROMs), electrically erasable programmable read-only memories (EEPROMs), compact disks, DVDs, and/or the like. In accordance with some embodiments, memory may include one or more disk drives, flash drives, databases, local cache memories, processor cache memories, relational databases, flat databases, servers, cloud based platforms, and/or the like. In addition, those of ordinary skill in the art will appreciate many additional devices and techniques for storing information can be used as memory.

Memory may be used to store instructions for running one or more applications or modules on a processor. For example, memory could be used in some embodiments to house all or some of the instructions needed to execute the functionality of one or more of the modules and/or applications disclosed in this application.

### HTP Microbial Strain Engineering Based Upon Genetic Design Predictions: An Example Workflow

In some embodiments, the present disclosure teaches the directed engineering of new host organisms based on the recommendations of the computational analysis systems of the present disclosure.

In some embodiments, the present disclosure is compatible with all genetic design and cloning methods. That is, in some embodiments, the present disclosure teaches the use of traditional cloning techniques such as polymerase chain reaction, restriction enzyme digestions, ligation, homologous recombination, RT PCR, and others generally known in the art and are disclosed in for example: Sambrook et al. (2001) Molecular Cloning: A Laboratory Manual (3^{rd} ed., Cold Spring Harbor Laboratory Press, Plainview, N.Y.), incorporated herein by reference.

In some embodiments, the cloned sequences can include possibilities from any of the HTP genetic design libraries taught herein, for example: promoters from a promoter swap library, SNPs from a SNP swap library, start or stop codons from a start/stop codon exchange library, terminators from a STOP swap library, or sequence optimizations from a sequence optimization library.

Further, the exact sequence combinations that should be included in a particular construct can be informed by the epistatic mapping function.

In other embodiments, the cloned sequences can also include sequences based on rational design (hypothesis-driven) and/or sequences based on other sources, such as scientific publications.

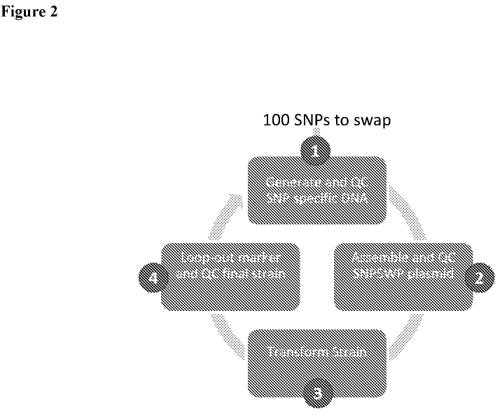

In some embodiments, the present disclosure teaches methods of directed engineering, including the steps of i) generating custom-made SNP-specific DNA, ii) assembling SNP-specific plasmids, iii) transforming target host cells with SNP-specific DNA, and iv) looping out any selection markers (See FIG. 2).

FIG. 6A depicts the general workflow of the strain engineering methods of the present disclosure, including acquiring and assembling DNA, assembling vectors, transforming host cells and removing selection markers.

Build Specific DNA Oligonucleotides

In some embodiments, the present disclosure teaches inserting and/or replacing and/or altering and/or deleting a DNA segment of the host cell organism. In some aspects, the methods taught herein involve building an oligonucleotide of interest (i.e. a target DNA segment), that will be incorporated into the genome of a host organism. In some embodiments, the target DNA segments of the present disclosure can be obtained via any method known in the art, including: copying or cutting from a known template, mutation, or DNA synthesis. In some embodiments, the present disclosure is compatible with commercially available gene synthesis products for producing target DNA sequences (e.g., GeneArt™, GeneMaker™, GenScript™, Anagen™ Blue Heron™, Entelechon™, GeNOsys, Inc., or Qiagen™)

In some embodiments, the target DNA segment is designed to incorporate a SNP into a selected DNA region of the host organism (e.g., adding a beneficial SNP). In other embodiments, the DNA segment is designed to remove a SNP from the DNA of the host organisms (e.g., removing a detrimental or neutral SNP).

In some embodiments, the oligonucleotides used in the inventive methods can be synthesized using any of the methods of enzymatic or chemical synthesis known in the art. The oligonucleotides may be synthesized on solid supports such as controlled pore glass (CPG), polystyrene beads, or membranes composed of thermoplastic polymers that may contain CPG. Oligonucleotides can also be synthesized on arrays, on a parallel microscale using microfluidics (Tian et al., Mol. BioSyst., 5, 714-722 (2009)), or known technologies that offer combinations of both (see Jacobsen et al., U.S. Pat. App. No. 2011/0172127).

Synthesis on arrays or through microfluidics offers an advantage over conventional solid support synthesis by reducing costs through lower reagent use. The scale required for gene synthesis is low, so the scale of oligonucleotide product synthesized from arrays or through microfluidics is acceptable. However, the synthesized oligonucleotides are of lesser quality than when using solid support synthesis (See Tian infra.; see also Staehler et al., U.S. Pat. App. No. 2010/0216648).

A great number of advances have been achieved in the traditional four-step phosphoramidite chemistry since it was first described in the 1980s (see for example, Sierzchala, et al. J. Am. Chem. Soc., 125, 13427-13441 (2003) using peroxy anion deprotection; Hayakawa et al., U.S. Pat. No. 6,040,439 for alternative protecting groups; Azhayev et al, Tetrahedron 57, 4977-4986 (2001) for universal supports; Kozlov et al., Nucleosides, Nucleotides, and Nucleic Acids, 24 (5-7), 1037-1041 (2005) for improved synthesis of longer oligonucleotides through the use of large-pore CPG; and Damha et al., NAR, 18, 3813-3821 (1990) for improved derivatization).

Regardless of the type of synthesis, the resulting oligonucleotides may then form the smaller building blocks for longer oligonucleotides. In some embodiments, smaller oligonucleotides can be joined together using protocols known in the art, such as polymerase chain assembly (PCA), ligase chain reaction (LCR), and thermodynamically balanced inside-out synthesis (TBIO) (see Czar et al. Trends in Biotechnology, 27, 63-71 (2009)). In PCA, oligonucleotides spanning the entire length of the desired longer product are annealed and extended in multiple cycles (typically about 55 cycles) to eventually achieve full-length product. LCR uses ligase enzyme to join two oligonucleotides that are both annealed to a third oligonucleotide. TBIO synthesis starts at the center of the desired product and is progressively extended in both directions by using overlapping oligonucleotides that are homologous to the forward strand at the 5′ end of the gene and against the reverse strand at the 3′ end of the gene.

Another method of synthesizing a larger double stranded DNA fragment is to combine smaller oligonucleotides through top-strand PCR (TSP). In this method, a plurality of oligonucleotides spans the entire length of a desired product and contain overlapping regions to the adjacent oligonucleotide(s). Amplification can be performed with universal forward and reverse primers, and through multiple cycles of amplification a full-length double stranded DNA product is formed. This product can then undergo optional error correction and further amplification that results in the desired double stranded DNA fragment end product.

In one method of TSP, the set of smaller oligonucleotides that will be combined to form the full-length desired product are between 40-200 bases long and overlap each other by at least about 15-20 bases. For practical purposes, the overlap region should be at a minimum long enough to ensure specific annealing of oligonucleotides and have a high enough melting temperature (Tₘ) to anneal at the reaction temperature employed. The overlap can extend to the point where a given oligonucleotide is completely overlapped by adjacent oligonucleotides. The amount of overlap does not seem to have any effect on the quality of the final product. The first and last oligonucleotide building block in the assembly should contain binding sites for forward and reverse amplification primers. In one embodiment, the terminal end sequence of the first and last oligonucleotide contain the same sequence of complementarity to allow for the use of universal primers.

Assembling/Cloning Custom Plasmids

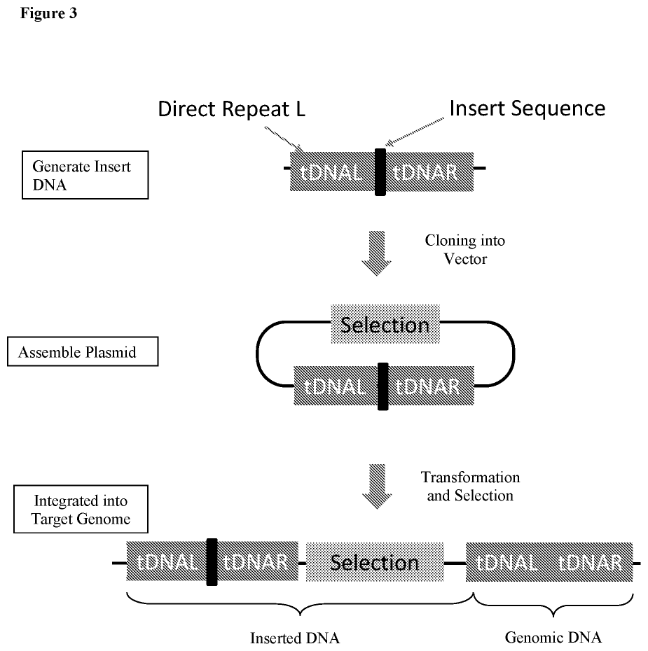

In some embodiments, the present disclosure teaches methods for constructing vectors capable of inserting desired target DNA sections (e.g. containing a particular SNP) into the genome of host organisms. In some embodiments, the present disclosure teaches methods of cloning vectors comprising the target DNA, homology arms, and at least one selection marker (see FIG. 3).

In some embodiments, the present disclosure is compatible with any vector suited for transformation into the host organism. In some embodiments, the present disclosure teaches use of shuttle vectors compatible with a host cell. In one embodiment, a shuttle vector for use in the methods provided herein is a shuttle vector compatible with an E. coli and/or Saccharopolyspora host cell. Shuttle vectors for use in the methods provided herein can comprise markers for selection and/or counter-selection as described herein. The markers can be any markers known in the art and/or provided herein. The shuttle vectors can further comprise any regulatory sequence(s) and/or sequences useful in the assembly of said shuttle vectors as known in the art. The shuttle vectors can further comprise any origins of replication that may be needed for propagation in a host cell as provided herein such as, for example, E. coli or C. glutamicum. The regulatory sequence can be any regulatory sequence known in the art or provided herein such as, for example, a promoter, start, stop, signal, secretion and/or termination sequence used by the genetic machinery of the host cell. In certain instances, the target DNA can be inserted into vectors, constructs or plasmids obtainable from any repository or catalogue product, such as a commercial vector (see e.g., DNA2.0 custom or GATEWAY® vectors). In certain instances, the target DNA can be inserted into vectors, constructs or plasmids obtainable from any repository or catalogue product, such as a commercial vector (see e.g., DNA2.0 custom or GATEWAY® vectors).

In some embodiments, the assembly/cloning methods of the present disclosure may employ at least one of the following assembly strategies: i) type II conventional cloning, ii) type II S-mediated or “Golden Gate” cloning (see, e.g., Engler, C., R. Kandzia, and S. Marillonnet. 2008 “A one pot, one step, precision cloning method with high-throughput capability”. PLos One 3:e3647; Kotera, I., and T. Nagai. 2008 “A high-throughput and single-tube recombination of crude PCR products using a DNA polymerase inhibitor and type IIS restriction enzyme.” J Biotechnol 137:1-7; Weber, E., R. Gruetzner, S. Werner, C. Engler, and S. Marillonnet. 2011 Assembly of Designer TAL Effectors by Golden Gate Cloning. PloS One 6:e19722), iii) GATEWAY® recombination, iv) TOPO® cloning, exonuclease-mediated assembly (Aslanidis and de Jong 1990. “Ligation-independent cloning of PCR products (LIC-PCR).” Nucleic Acids Research, Vol. 18, No. 20 6069), v) homologous recombination, vi) non-homologous end joining, vii) Gibson assembly (Gibson et al., 2009 “Enzymatic assembly of DNA molecules up to several hundred kilobases” Nature Methods 6, 343-345) or a combination thereof. Modular type IIS based assembly strategies are disclosed in PCT Publication WO 2011/154147, the disclosure of which is incorporated herein by reference.

In some embodiments, the present disclosure teaches cloning vectors with at least one selection marker. Various selection marker genes are known in the art often encoding antibiotic resistance function for selection in prokaryotic (e.g., against ampicillin, kanamycin, tetracycline, chloramphenicol, zeocin, spectinomycin/streptomycin) or eukaryotic cells (e.g. geneticin, neomycin, hygromycin, puromycin, blasticidin, zeocin) under selective pressure. Other marker systems allow for screening and identification of wanted or unwanted cells such as the well-known blue/white screening system used in bacteria to select positive clones in the presence of X-gal or fluorescent reporters such as green or red fluorescent proteins expressed in successfully transduced host cells. Another class of selection markers most of which are only functional in prokaryotic systems relates to counter selectable marker genes often also referred to as “death genes” which express toxic gene products that kill producer cells. Examples of such genes include sacB, rpsL(strA), tetAR, pheS, thyA, gata-1, or ccdB, the function of which is described in (Reyrat et al. 1998 “Counterselectable Markers: Untapped Tools for Bacterial Genetics and Pathogenesis.” Infect Immun. 66(9): 4011-4017).

Counter-Selection Markers

The present disclosure also provides counterselection marker for genetic engineering of Saccharopolyspora spp. In some embodiments, the Saccharopolyspora spp. is Saccharopolyspora spinosa. In some embodiments, the counterselection marker is a sacB (levansucrase) gene encoding levansucrase (EC 2.4.1.10), a phenylalanine tRNA synthetase (pheS) gene, or combinations thereof.

In some embodiments, a nucleotide sequence encoding sacB or pheS gene is codon-optimized for Saccharopolyspora spp., such as Saccharopolyspora spinosa. In some embodiments, the nucleotide sequence encoding sacB comprises SEQ ID No. 146. In some embodiments, the nucleotide sequence encoding sacB has at least 70%, 75%, 80%, 85%, 90%, 95%, 96%, 97%, 98%, 99%, or more homology to SEQ ID No. 146. In some embodiments, the nucleotide sequence encoding pheS comprises SEQ ID No. 147 or SEQ ID No. 148. In some embodiments, the nucleotide sequence encoding pheS has at least 70%, 75%, 80%, 85%, 90%, 95%, 96%, 97%, 98%, 99%, or more homology to SEQ ID No. 147 or SEQ ID No. 148.

Also provided are plasmids for genomic integration for Saccharopolyspora spp. comprising a counterselection marker gene of the present disclosure. In some embodiments, the plasmids comprise plasmid backbone, a positive selection marker in addition to the counterselection marker gene, homologous left arm sequence, homologous right arm sequence, and DNA payload (e.g., edited gene to be integrated). The homologous left and right arm sequences enables of homologous recombination between the targeted wild type locus and the DNA payload. In some embodiments, the counterselection marker is a sacB gene or a pheS gene.

Also provided are methods of generating mutant strains of Saccharopolyspora spp. In some embodiments, the methods comprise a) introducing a plasmid comprising a counterselection marker gene of the present disclosure into a parent Saccharopolyspora strain. This can be done by using homologous recombination or any other suitable process. In some embodiments, the methods further comprise b) selecting for strains with integration event using a positive selection (e.g., based on the positive selection marker in the plasmid. In some embodiments, the methods further comprise selecting for strains having the plasmid backbone looped out using a negative selection (e.g., based on the counterselection marker gene). In some embodiments, the resulted Saccharopolyspora strain has better performance compared to the parent strain without the integrated DNA. In some embodiments, the counterselection marker is a sacB gene or a pheS gene.

Levansucrase (EC 2.4.1.10) is an enzyme that catalyzes the chemical reaction

sucrose+(2,6-beta-D-fructosyl)n [symbol] {\displaystyle \rightleftharpoons}\rightleftharpoons glucose+(2,6-beta-D-fructosyl)n+1

The two substrates of this enzyme are sucrose and (2,6-beta-D-fructosyl)n, whereas its two products are glucose and (2,6-beta-D-fructosyl)n+1. This enzyme belongs to the family of glycosyltransferases, specifically the hexosyltransferases. The systematic name of this enzyme class is sucrose:2,6-beta-D-fructan 6-beta-D-fructosyltransferase. Other names in common use include sucrose 6-fructosyltransferase, beta-2,6-fructosyltransferase, and beta-2,6-fructan:D-glucose 1-fructosyltransferase.

### Scarless Targeted Genomic Editing in Saccharopolyspora Strains

Also provided are methods of targeted genomic editing in Saccharopolyspora strain, such as a Saccharopolyspora spinose strain. The methods result in a scarless Saccharopolyspora strain containing a genetic variation at a targeted genomic locus.

In some embodiments, the methods comprise (a) introducing a genomic editing plasmid into a Saccharopolyspora strain. Said genomic editing plasmid comprises (1) a selection marker; (2) a counterselection marker, (3) a DNA fragment bearing one or more desired genetic variations to be introduced into the genome of the; and (4) plasmid backbone sequence. In some embodiments, the DNA fragment bearing one or more desired genetic variations comprises one more genetic variations to be integrated into the Saccharopolyspora genome at a target locus, and homology arms to the target genomic locus flanking the desired genetic variations.

In some embodiments, the methods further comprise (b) selecting for a Saccharopolyspora strain that has undergone an initial homologous recombination and has the genetic variation integrated into the target locus based on the presence of the selection marker in the genome.

In some embodiments, the methods further comprise (c) selecting for a Saccharopolyspora strain that has the genetic variation integrated into the target locus, but has undergone an additional homologous recombination that loops-out the plasmid backbone, based on the absence of the counterselection marker. In some embodiments, the counterselection marker is selected from those described in the present disclosure.

In some embodiments, step (b) and step (c) of the methods are performed simultaneously on same medium. In some embodiments, step (b) and step (c) of the methods are performed sequentially on separate media.

In some embodiments, the targeted genomic locus may comprise any region of the Saccharopolyspora genome, including genomic regions that do not contain repeating segments of encoding DNA modules.

In some embodiments, the genomic editing plasmid does not comprise a temperature sensitive replicon that is functional in the Saccharopolyspora strain.

In some embodiments, the genomic editing plasmid does not comprise an origin of replication that enables self-replication of the plasmid within the Saccharopolyspora strain.

In some embodiments, the selection step (c) is performed without replication of the integrated plasmid.

In some embodiments, the genomic editing plasmid in a Saccharopolyspora strain is introduced into the Saccharopolyspora strain using the conjugation method as described in the present disclosure. In some embodiments, the donor cell delivering the genomic editing plasmid is a E. coli cell. In some embodiments, the recipient cell is a Saccharopolyspora spinosa cell. Alternatively, in some embodiments, the genomic editing plasmid is directly transformed into a Saccharopolyspora strain.

Various homologous recombination plasmids can be used. In some embodiments, the genomic editing plasmid is a single homologous recombination vector. A single homologous recombination plasmid can comprise an “insertion cassette.” An insertion homologous recombination cassette comprises a single region sharing sufficient sequence identity to a target site which promotes a single homologous recombination cross-over event. In specific embodiments, the insertion cassette further comprises a polynucleotide of interest. As only a single cross-over event occurs, the entire insertion cassette—and the plasmid/vector it is contained in—is integrated at the target site. Such insertion cassettes are generally contained on circular vectors/plasmids. See, U.S. Publications 2003/0131370, 2003/0157076, 2003/0188325, and 2004/0107452, Thomas et al. (1987) Cell 51:503-512, and Pennington et al. (1991) Proc. Natl. Acad. Sci. USA 88:9498-9502, each of which is herein incorporated by reference in its entirety.

In some embodiments, the genomic editing plasmid is a double homologous recombination vector. For example, the homologous recombination cassette comprises a “replacement vector.” Replacement homologous recombination cassettes comprise a first and a second region having sufficient sequence identity to a corresponding first and second region of a target site in a eukaryotic cell. A double homologous recombination cross-over event occurs and any polynucleotide internal to the first and second region is integrated at the target site (i.e., homologous recombination between the first region of homology of the cassette and the corresponding first region of the target site and homologous recombination between the second region of homology of the recombination cassette and the corresponding second region of the target site). See, Yang et al. (2014) Applied and Environmental Microbiology 80:3826-3834, Posfai et al. (1999) Nucleic Acids Research 27(2):4409-4415; Graf et al. (2011) Applied and Environmental Microbiology 77:5549-5552, each of which is herein incorporated by reference in its entirety.

Protoplasting Methods

In one embodiment, the methods and systems provided herein make use of the generation of protoplasts from filamentous fungal cells. Suitable procedures for preparation of protoplasts can be any known in the art including, for example, those described in EP 238,023 and Yelton et al. (1984, Proc. Natl. Acad. Sci. USA 81:1470-1474). In one embodiment, protoplasts are generated by treating a culture of filamentous fungal cells with one or more lytic enzymes or a mixture thereof. The lytic enzymes can be a beta-glucanase and/or a polygalacturonase. In one embodiment, the enzyme mixture for generating protoplasts is VinoTaste concentrate. Following enzymatic treatment, the protoplasts can be isolated using methods known in the art such as, for example, centrifugation.

The pre-cultivation and the actual protoplasting step can be varied to optimize the number of protoplasts and the transformation efficiency. For example, there can be variations of inoculum size, inoculum method, pre-cultivation media, pre-cultivation times, pre-cultivation temperatures, mixing conditions, washing buffer composition, dilution ratios, buffer composition during lytic enzyme treatment, the type and/or concentration of lytic enzyme used, the time of incubation with lytic enzyme, the protoplast washing procedures and/or buffers, the concentration of protoplasts and/or polynucleotide and/or transformation reagents during the actual transformation, the physical parameters during the transformation, the procedures following the transformation up to the obtained transformants.

The present disclosure also provides a method for rapid consolidation of genetic changes in two or more microbial strains and for generating genetic diversity in Saccharopolyspora spp. based on protoplast fusion. In some embodiments, when at least one of the microbial strains contains a “marked” mutation, the method comprises the following steps: (1) choosing parent strains from a pool of engineered strains for consolidation; (2) preparing protoplasts (e.g., removing the cell wall, etc.) from the strains that are to be consolidated; and (3) fusing the strains of interest; (4) recovering of cells. (5) selecting cells which carry the “marked” mutation, and (6) genotyping growing cells for the presence of mutations coming for the other parent strains. Optionally, the method further comprises the step of (7) removing the plasmid form the “marked” mutation. In some embodiments, when none of the microbial strains contains a “marked” mutation, the method comprises the following steps: (1) choosing parent strains from a pool of engineered strains for consolidation; (2) preparing protoplasts (e.g., removing the cell wall, etc.) from the strains that are to be consolidated; and (3) fusing the strains of interest; (4) recovering of cells. (5) selecting cells for the presence of mutations coming from the first parent strain, and (6) selecting cells for the presence of mutations coming for the other parent strains. In some embodiments, the strains are selected based on a phenotype associated with the mutation coming from the first parent strain and/or from the other parent strain. In some embodiments, the strains are selected based on genotyping. In some embodiments, the genotyping step is done in a high-throughput procedure.

The method as described herein is extremely efficient compared to traditional methods. For example, the traditional way of combining mutations in Saccharopolyspora spp. is to generate the first mutation into a base strain through integration and counter-selection (˜45 days)) thus generating a mutant strain (Mut1 for example) and then proceed to repeat the process with the next mutation using the Mut1 strain as a recipient and going through the 45 day engineering process again thus generating a new strain with two mutations (e.g. Mut2). However, the method of the present disclosure only requires about less than 14 days, 15 days, 16 days, 17 days, 18 days, 19 days, 20 days, or 21 days to reach the same strain.

In some embodiments, in step (3), to increase the odds of generating useful (novel) combinations of mutants, fewer cells of the stain with “marked” mutation can be used, thus increasing the chances that these “marked” cells would have interacted and fused with cells carrying different mutations. In some embodiments, the ratio of cells of the stain with “marked” mutation to cells of the stain with “unmarked” mutation is about 1:1.5, 1:2, 1:3, 1:4, 1:5, 1:6, 1:7, 1:8, 1:9, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:200, 1:300, 1:400, 1:500, 1:600, 1:700, 1:800, 1:900, 1:1000, or more.

In some embodiments, in step (4), cells are plated on osmotically stabilized media without the use of agar overlay, which simplifies the procedure and allows for easier automation. The osmo-stabilizers are such that allow for the growth of cells which might contain the counter-selection marker gene (e.g., sacB gene). Protoplasted cells are very sensitive to treatment and are easy to kill. This step ensures that enough cells are recovered. The better this step works, the more material can be used for downstream analysis.

In some embodiments, in step (5), the step is accomplished by overlaying appropriate antibiotic onto the growing cells. In case neither of the parent cell carries a “marked” mutation, the strains can be genotyped by other means to identify strains of interest. This step could be optional but it ensures that cells that have most likely undergone cell fusion are enriched. It is possible to “mark” multiple loci and this way one can generate the combinations of interest faster, but then multiple plasmids may have to be removed if one would like to have “scarless” strains.

In some embodiments, in step (6), the number of colonies to genotype depends on the complexity of the cross as well as the selection scheme.

In some embodiments, step (7) is optional and is recommended for additional verification or client delivery. In some embodiments, at the end of engineering cycles for a strain, all plasmid remnants need to be removed. When and how often this is carried out is at the discretion of the user. In some embodiments, the presence of the counter-selectable sacB gene makes this step straightforward. In some embodiments, at least one of the stains has a “marked” mutation. In some embodiments, the number of strains fused during a single consolidation step can be two or more, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, 500, or more. In some embodiments, one or more of the strain for fusing can be tagged by a selection marker at loci of interest. In some embodiments, when one of the parental strain comprises a genetic mutation that is “marked”, while the genetic mutation in the other parental strain is unmarked, the ratio of unmarked strain vs. marked strain is about 10:1, 20:1, 30:1, 40:1, 50:1, 60:1, 70:1, 80:1, 90:1, 100:1, 150:1, 200:1, 250:1, 300:1, or more. In some embodiments, when the parental population has more than 5, 6, 7, 8, 9, 10, 15, 20, 25, 30, 35, 40, 45, 50 or more unmarked strains, equal proportions of each are used. In some embodiments, when live unmarked strain and dead marked strain and are used, the ration of live:dead is about 1:1 or about 1:2 (live:dead).

The methods of the present disclosure contain important improvements compared to method described previously (Practical Streptomyces Genetics, ISBN 0-7084-0623-8). Such improvements include, but are not limited to:
An initial centrifugations for protoplast generation is conducted at higher speed (5000×g vs 1000×g) for shorter time 5 min vs 10 min. This shortens the time required to complete the protocol;
In some embodiments, a YEME media with modified composition is used to accommodate the use of strains with sacB gene. Typical YEME compositions includes sucrose, which his not tolerated by strains with sacB gene. Our modified YEME media substitute sucrose with 1M sorbitol;
In some embodiments, there is no filtration step of running digested cells through cotton wool to separate mycelia from protoplasts. In some embodiments, there are no mycelia left after the enzymatic treatment, so the step is not needed;
In some embodiments, protoplasts are resuspend the produced in about 1:5, 1:6, 1:7, 1:8, 1:9, 1:10, 1:15, 1:20, or less of the volume recommended in Practical Streptomyces Genetics (ISBN 0-7084-0623-8), to remove a subsequent spin step and to make it easier for automation;
In some embodiments, fused protoplasts are recover in a R2YE broth rather than top-agar. This greatly simplifies automation and handling. Agar can solidify and clog tips and needs to be kept worm during the protocol. Broth does not have these complications. This modification does not greatly reduce protoplast viability.
In some embodiments, protoplasts are recovered on R2YE media supplemented with 0.5M sorbitol and 0.5M mannose. This formulation required time and experimentation to develop. The inventors originally tried to use only sorbitol at 1M or 0.5M but it was not effective in stabilizing the protoplasts and cells grow slow in the presence of 1M sorbitol. However, the inventors found out that if the media is supplemented with sorbitol and manose (0.5M each), it works better as an osmotic stabilization media.

In some embodiments, in step (2), cell wall is removed by lysozyme treatment. In some embodiments, about 1 mg/ml, 2 mg/ml. 3 mg/ml, 4 mg/ml, 5 mg/ml, 6 mg/ml, 7 mg/ml, 8 mg/ml, 9 mg/ml, or 10 mg/ml lysozyme in sterile P-buffer is used. In some embodiments, the total incubation time is about 70 min, 75 min, 80 min, 85 min, 90 min, 95 min, or 100 min at 37° C. In some embodiments, the resulted protoplasts are validated by evaluating whether they are lysed by water. In some embodiments, one can determine water sensitivity by microscopy and by outgrowth on osmo-stabilized media.

Transformation of Host Cells

In some embodiments, the vectors of the present disclosure may be introduced into the host cells using any of a variety of techniques, including transformation, transfection, transduction, viral infection, gene guns, or Ti-mediated gene transfer (see Christie, P. J., and Gordon, J. E., 2014 “The Agrobacterium Ti Plasmids” Microbiol SPectr. 2014; 2(6); 10.1128). Particular methods include calcium phosphate transfection, DEAE-Dextran mediated transfection, lipofection, or electroporation (Davis, L., Dibner, M., Battey, I., 1986 “Basic Methods in Molecular Biology”). Other methods of transformation include for example, lithium acetate transformation and electroporation See, e.g., Gietz et al., Nucleic Acids Res. 27:69-74 (1992); Ito et al., J. Bacterol. 153:163-168 (1983); and Becker and Guarente, Methods in Enzymology 194:182-187 (1991). In some embodiments, transformed host cells are referred to as recombinant host strains.

In some embodiments, the present disclosure teaches high-throughput transformation of cells using the 96-well plate robotics platform and liquid handling machines of the present disclosure.

In some embodiments, the present disclosure teaches screening transformed cells with one or more selection markers as described above. In one such embodiment, cells transformed with a vector comprising a kanamycin resistance marker (KanR) are plated on media containing effective amounts of the kanamycin antibiotic. Colony forming units visible on kanamycin-laced media are presumed to have incorporated the vector cassette into their genome. Insertion of the desired sequences can be confirmed via PCR, restriction enzyme analysis, and/or sequencing of the relevant insertion site.

Looping Out of Selected Sequences

In some embodiments, the present disclosure teaches methods of looping out selected regions of DNA from the host organisms. The looping out method can be as described in Nakashima et al. 2014 “Bacterial Cellular Engineering by Genome Editing and Gene Silencing.” Int. J. Mol. Sci. 15(2), 2773-2793. In some embodiments, the present disclosure teaches looping out selection markers from positive transformants. Looping out deletion techniques are known in the art, and are described in (Tear et al. 2014 “Excision of Unstable Artificial Gene-Specific inverted Repeats Mediates Scar-Free Gene Deletions in Escherichia coli.” Appl. Biochem. Biotech. 175:1858-1867). The looping out methods used in the methods provided herein can be performed using single-crossover homologous recombination or double-crossover homologous recombination. In one embodiment, looping out of selected regions as described herein can entail using single-crossover homologous recombination as described herein.

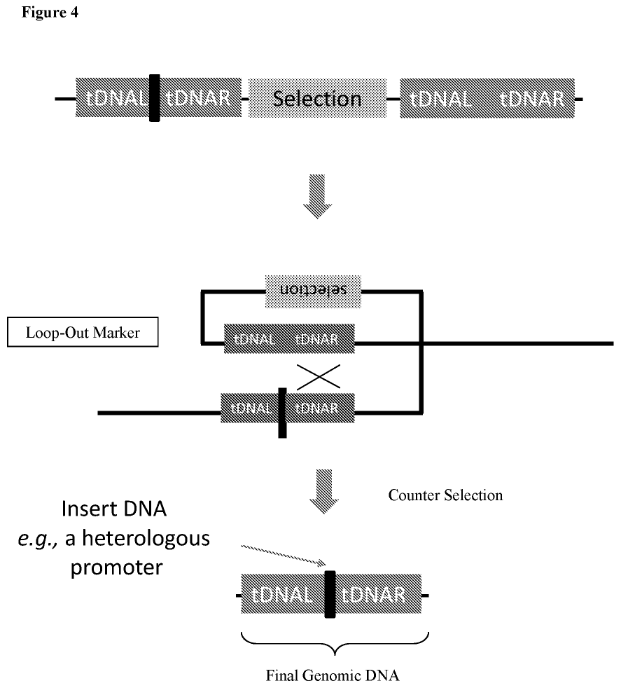

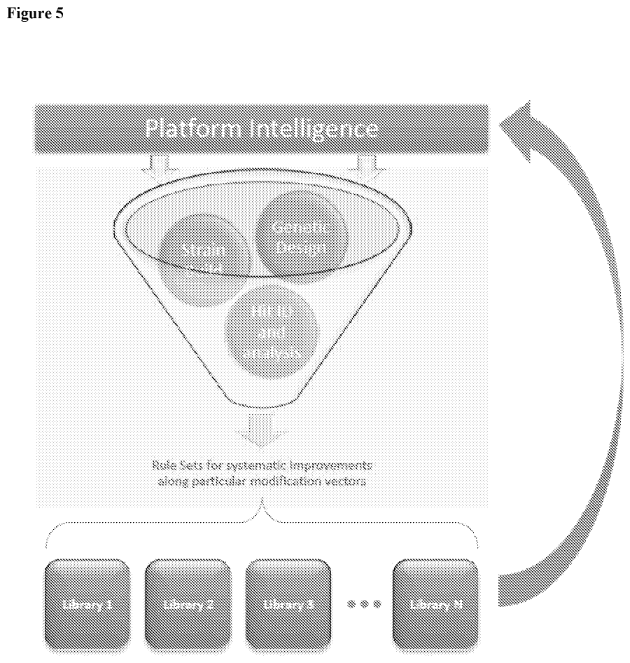

First, loop out vectors are inserted into selected target regions within the genome of the host organism (e.g., via homologous recombination, CRISPR, or other gene editing technique). In one embodiment, single-crossover homologous recombination is used between a circular plasmid or vector and the host cell genome in order to loop-in the circular plasmid or vector such as depicted in FIG. 3. The inserted vector can be designed with a sequence which is a direct repeat of an existing or introduced nearby host sequence, such that the direct repeats flank the region of DNA slated for looping and deletion. Once inserted, cells containing the loop out plasmid or vector can be counter selected for deletion of the selection region (e.g., see FIG. 4; lack of resistance to the selection gene).

Persons having skill in the art will recognize that the description of the loopout procedure represents but one illustrative method for deleting unwanted regions from a genome. Indeed the methods of the present disclosure are compatible with any method for genome deletions, including but not limited to gene editing via CRISPR, TALENS, FOK, or other endonucleases. Persons skilled in the art will also recognize the ability to replace unwanted regions of the genome via homologous recombination techniques.

Neutral Integration Sites

Foreign genes and even entire pathways are often ported into chassis organisms, requiring either plasmid-based expression or identification of a neutral site for genome integration. As genome integration is more stable and predictable compared to plasmid-based expression, this is often the preferred method for modification, particularly for industrial microbial strains.

These neutral integration sites are genetic loci into which individual genes or multi-gene cassettes can be stably and efficiently integrated within the genome of a microbial strains, such as Saccharopolyspora spp. strains. Integration of sequences into these sites have no or limited effect on growth of the strains. As used herein, “neutral integration site” refers to a gene or chromosomal locus, natively present on the chromosome of a microbial cell, whose normal function is not required for the growth of the cell or for the capability of the cell to perform all the functions for a certain biological process. When disrupted by the integration of a DNA sequence not normally present within that gene, the cell harboring a disrupted neutral integration site gene can productively perform the biological process.

In some embodiments, the present disclosure provides neutral integration sites (NISs) in S. spinosa. Such neutral integration sites include, but are not limited to a locus having the sequence of any one of SEQ ID No. 132 to SEQ ID No. 142. These NISs may be conservative among all Saccharopolyspora spp. Thus, loci in Saccharopolyspora spp. other than S. spinosa but sharing homology to the NISs in S. spinosa are also potential neutral integration sites.

Such neutral integration sites have multiple utilities. For example, exogenous DNA fragment having relatively large size can be inserted into a single neutral integration site described herein. Such DNA fragment may have a size of at least 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 25 kb, 30 kb, 40 kb, 50 kb, 60 kb, 70 kb, 80 kb, 90 kb, 100 kb, or more, without affecting the growth of the host cell.

DNA fragment to be integrated into the NISs can be any desired sequence. Such DNA fragment to be integrated may bring new function to the host cell, enhance existing function of the host cell, or reduce the effect of any factor that may negatively affect the host cell grow. For example, Saccharopolyspora spp. strains having genetic element(s) inserted into the neutral integration site(s) may have improved performance (e.g., improved yield of one or more molecules of interest, such as a spinosyn) compared to a reference strain that does not have the insertion.

In some embodiments, the DNA fragment to be integrated comprises sequence homologous and/or heterologous to the host cell. In some embodiments, the DNA fragment to be integrated comprises a selected promoter that is functional in the host cell. In some embodiments, the DNA fragment to be integrated comprises a selected terminator sequence that is functional in the host cell. In some embodiments, the promoters and terminator sequences can be any of the sequences described in the present disclosure, or those known in the field.

In some embodiments, the DNA fragment to be integrated comprises one or more selection marker, which can be used to select for cells comprising the integrated DNA fragment. In some embodiments, the DNA fragment to be integrated comprises a counter-selection marker, which can be used to facilitate loop-out of full or part of the integrated DNA fragment.

In some embodiments, one or more exogenous genes can be integrated into the NIS of Saccharopolyspora spp. as described in the present disclosure, to introduce novel function into the microbial species, such as establishing a novel pathway. In some embodiments, such a novel pathway is a synthetic pathway and/or a signaling transduction pathway that does not exist in natural host cell. In some embodiments the DNA fragment to be integrated contains an attachment site for an integrase, allowing subsequent, efficient, targeted integration of biosynthetic pathways or components thereof. In some embodiments, a DNA fragment comprising a whole gene cluster or a part of a gene cluster encoding one or more gene product(s) that is (are) part of a biosynthetic pathway for secondary metabolites is integrated into a NIS of the present disclosure. Secondary metabolites often play an important role in plant defense against herbivory and other interspecies defenses. Secondary metabolites can have a role in the struggle for nutrients and habitat in a complex microbial environment. In some embodiments, secondary metabolites have biological activity against competing bacteria, fungi, yeast or other organisms. In some embodiments, the secondary metabolites are acting as inhibitors of competitor's nutrient uptake enzymes, or directly display antibacterial or antifungal activity. In some embodiments, the secondary metabolite counters competitor's defence mechanisms and yet others counter competitor's offence mechanism. It is well known that secondary metabolites show incredible wealth of diversity in terms of chemical characteristics. Therefore, humans use some secondary metabolites as medicines, flavorings, and recreational drugs. Secondary metabolites can be divided in the following categories: Small “small molecules”, such as beta-lactams, alkaloids, terpenoids, glycosides, natural phenols, phenazines, biphenyls and dibenzufurans; big “small molecules”, produced by large, modular, “molecular factories”, such as polyketides, complex glycosides, nonribosomal peptides, and hybrids of the above three; and non-“small molecules”—DNA, RNA, ribosome, or polysaccharide “classical” biopolymers, such as ribosomal peptides.

In some embodiments, a NIS of the present disclosure can be incorporated into a vector. A “vector” is a replicon, such as plasmid, phage, bacterial artificial chromosome (BAC) or cosmid, to which another DNA segment (e.g. a foreign gene) may be incorporated so as to bring about the replication of the attached segment, resulting in expression of the introduced sequence. Vectors may comprise a promoter and one or more control elements (e.g., enhancer elements) that are heterologous to the introduced DNA but are recognized and used by the host cell. In some embodiments, said vector can be further incorporated into genome of a different microbial species, thus establishing a NIS in the different microbial species. For example, a NIS of Saccharopolyspora spinosa described in the present disclosure can be incorporated into the genome of a related Saccharopolyspora species.

Integrase

An enzyme called “integrase” recognizes two attachment (att) sites (conserved nucleotide sequences typically located within tRNA genes in the host chromosome), joins the two DNA molecules and catalyzes a DNA double-strand breakage. A rejoining event results in the integration of one of the DNA molecules into the other DNA of the recipient cell. (N. D. Grindley, K. L. Whiteson, P. A. Rice, 2006. Annu. Rev. Biochem. 75, 567-605.) Therefore, integrases can direct target integration of DNA payloads through recognition and attachment at conserved sites.

The present disclosure provides compositions and methods for targeted cloning and/or transferring of DNA fragments from a donor organism into a host cell. In some embodiments, the host cell to be modified comprises sequences identical to, or having homology to att sites that can be recognized by a given integrase. In some embodiments, the host cell to be modified does not comprise sequences identical to, or having homology to att sites that can be recognized by a given integrase. In the second scenarios, sequences identical to, or having homology to att sites can be first inserted in to a neutral integration site in the host cell, such as a NIS described in the present disclosure.

In some embodiments, the integrase is derived from a Saccharopolyspora species. In some embodiments, the integrase is derived from S. endophytica, S. erythraea, or S. spinosa. In some embodiments, the integrase comprises the sequence of SEQ ID Nos 85, 87, 89, 91, 93, or any functional variants thereof.

In some embodiments, the integrase recognizes att sites that are derived from a Saccharopolyspora species. In some embodiments, the att sites are derived from S. endophytica, S. erythraea, or S. spinosa. In some embodiments, the integrase attachment site comprises the sequence of SEQ ID Nos. 167 to 171, or any functional variants thereof.

In some embodiments, DNA fragment to be integrated into the genome of a host cell has a size of at least 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 25 kb, 30 kb, 40 kb, 50 kb, 60 kb, 70 kb, 80 kb, 90 kb, 100 kb, or more.

The present disclosure provides vectors for integrating exogenous DNA into the genome of a host cell, such as a Saccharopolyspora species.

In some embodiments, the vectors comprise sequence(s) encoding an excisionase (xis), an integrase (int), and/or attachment site (attP). In some embodiments, sequence(s) in said vector are derived from S. endophytica. In some embodiments, the vectors are based on pCM32 as described by Chen et al. (“Characterization of the chromosomal integration of Saccharopolyspora plasmid pCM32 and its application to improve production of spinosyn in Saccharopolyspora spinosa.” Applied Microbiology and Biotechnology. PMID 26260388 DOI: 10.1007/s00253-015-6871-z). In some embodiments, sequence(s) in said vector are derived from S. erythraea. In some embodiments, the vectors are based on pSE101 and/or pSE211 as described by Te Poele et al. (“Actinomycete integrative and conjugative elements.” Antonie Van Leeuwenhoek 94, 127-143).

In some embodiments, the vectors of the present disclosure recognize a sequence in the genome of Saccharopolyspora spinosa. In some embodiments, the sequence in the genome of Saccharopolyspora spinosa that can be recognized by an integrase of the present disclosure has the sequence selected from SEQ ID Nos. 167 to 171, or any functional variants thereof. In some embodiments, an att site derived from S. endophytica and/or S. erythraea is introduced into the genome of Saccharopolyspora spinosa. In some embodiments, an att site derived from S. endophytica and/or S. erythraea is introduced into a NIS of Saccharopolyspora spinosa, such as any of those described in the present disclosure.

Additional tools and methods for using integrase are described in WO/2001/051639A2, WO/2013/189843A1, WO/2001/087936A2, WO/2001/083803A1, WO/2001/075116A2, and U.S. Pat. No. 6,569,668, each of which is herein incorporated by reference in its entirety.

Origins of Replication

The present disclosure also provides origins of replication and replicative elements for self-replicating plasmid system that can be used for a Saccharopolyspora species, such as Saccharopolyspora spinosa.

In some embodiments, origins and elements of self-replication enhance the types of genetic engineering and screening that can be performed in Saccharopolyspora spp. In some embodiments, the origins of self-replication are derived from the putative chromosomal origin of replication from S. erythraea (SEQ ID No. 94). In some embodiments, the origins of self-replication are derived from Actinomycete Integrative and Conjugative Elements (AICEs) in replicating plasmids pSE101 and pSE211 from S. erythraea (SEQ ID No. 95 and SEQ ID No. 96, respectively). In some embodiments, an origin for self-replicating of the present disclosure is assembled into a plasmid containing an antibiotic resistance marker, and with or without other genes required for self-replication (e.g., in case of AICEs). The assembled plasmid can be delivered to Saccharopolyspora spp., and antibiotic selection can be used to select for transformants having the self-replicating plasmid.

In some embodiments, an origin of self-replication of the present disclosure can be introduced into a Saccharopolyspora species, such as Saccharopolyspora spinosa. In some embodiments, a DNA fragment comprising the origin of replication has relatively large size, such as at least 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 25 kb, 30 kb, 40 kb, 50 kb, 60 kb, 70 kb, 80 kb, 90 kb, 100 kb, or more.

In some embodiments, DNA fragment comprising the origin of replication to be introduced into a Saccharopolyspora species can bring new function to the host cell, enhance existing function of the host cell, or reduce the effect of any factor that may negatively affect the host cell grow. For example, Saccharopolyspora spp. strains having genetic element(s) inserted into the genome may have improved performance (e.g., improved yield of one or more molecules of interest, such as a spinosyn) compared to a reference strain that does not have the insertion.

In some embodiments, the DNA fragment comprising the origin of replication to be introduced comprises sequence homologous and/or heterologous to the host cell. In some embodiments, the DNA fragment comprising the origin of replication to be introduced comprises a selected promoter that is functional in the host cell. In some embodiments, the DNA fragment to be introduced comprises a selected terminator sequence that is functional in the host cell. In some embodiments, the promoters and terminator sequences can be any of the sequences described in the present disclosure, or those known in the field.

In some embodiments, the DNA fragment comprising the origin of replication to be introduced comprises one or more selection marker, which can be used to select for cells comprising the DNA fragment. In some embodiments, the DNA fragment comprising the origin of replication to be introduced comprises a counter-selection marker, which can be used to facilitate loop-out of full or part of the DNA fragment.

In some embodiments, one or more exogenous genes can be introduced together with the origin of replication into Saccharopolyspora spp., to introduce novel function into the microbial species, such as establishing a novel pathway. In some embodiments, such a novel pathway is a synthetic pathway and/or a signaling transduction pathway that does not exist in natural host cell. In some embodiments, a DNA fragment comprising a whole gene cluster or a part of a gene cluster encoding one or more gene product(s) that is (are) part of a biosynthetic pathway for secondary metabolites.

Reporters

Saccharopolyspora is a largely intractable genus of hosts for which very few molecular biology tools have been established. These tools are extremely important for the development of engineering tools and engineering efforts. The present disclosure also provides reporter proteins and assays for Saccharopolyspora species, such as Saccharopolyspora spinosa. Thus, the present disclosure provides reporter system which has been lacking.

In some embodiments, provided are reporter proteins that are functional in Saccharopolyspora spp. In some embodiments, the reporter proteins are fluorescent proteins and enzyme beta-glucuronidase. In some embodiments, the fluorescent proteins are green fluorescent proteins and red fluorescent proteins. In some embodiments, the reporter proteins are Dasher GFP and Paprika RFP. (ATUM, https://www.atum.bio/products/protein-paintbox?exp=2) and the enzyme beta-glucuronidase (gusA) (Jefferson et al. (1986). “Beta-Glucuronidase from Escherichia coli as a gene-fusion marker”. Proceedings of the National Academy of Sciences of the United States of America. 83 (22): 8447-51).

In some embodiments, genes encoding a reporter protein is codon-optimized. In some embodiments, genes encoding the fluorescent proteins are codon-optimized for E. coli. In some embodiments, the genes encoding the fluorescent proteins have the nucleotide sequence of SEQ ID No. 81 or SEQ ID No. 82). In some embodiments, genes encoding the beta-glucuronidase (gusA) is codon-optimized for expression in S. spinosa, e.g., having the nucleotide sequence of SEQ ID No. 83.

In some embodiments, genes encoding a fluorescent protein is modified to change the fluorescent excitation and emission spectra of the reporter protein.

In some embodiments, two or more fluorescent proteins are used in a single Saccharopolyspora cell. In some embodiments, a green fluorescent protein and a red fluorescent protein are used in a single Saccharopolyspora cell. In some embodiments, the fluorescent excitation and emission spectra of the green fluorescent reporter protein and the red fluorescent reporter protein are distinct from each other.

In some embodiments, a reporter protein of the present disclosure is used to determine activity of a regulatory element for gene expression. In some embodiments, the regulatory element can be a promoter, a ribosomal binding site, a star/stop codon, a terminator, an enhancer, an suppressor, a single strand RNA, a double strand RNA, elements alike, or any combination thereof. For example, when a promoter is operably linked to a sequence encoding a reporter of the present disclosure and expressed in a microbial strain, the strength of the promoter in promoting gene expression can be determined by the fluorescent signal. Similarly, when a sequence encoding a reporter of the present disclosure is operably linked to a terminator sequence, the strength of the terminator in suppressing gene expression can be determined by the fluorescent signal. Thus, in some embodiments, the reporters are useful to determine the strength of a group of promoters, ribosomal binding sites, star/stop codons, terminators, enhancers, suppressors, single strand RNAs, double strand RNAs, and elements alike, thus establish a ladder (library). In some embodiments, a reporter protein of the present disclosure can be used as a screening tool. For example, strains with a given phenotype “marked” by the reporter protein can be sorted based on the presence or absence of the reporter protein, such as by flow cytometry, or observation on plate under excitation spectra.

In some embodiments, a reporter protein of the present disclosure can be fused to an endogenous or an exogenous polypeptide and expressed in Saccharopolyspora cells. In some embodiments, the reporter protein can be used in any way that a user desires.

In some embodiments, a gene encoding a reporter protein of the present disclosure can be linked to a terminator sequence. In some embodiments, the terminator has the sequence of SEQ ID No. 149.

### EXAMPLES

The following examples are given for the purpose of illustrating various embodiments of the disclosure and are not meant to limit the present disclosure in any fashion. Changes therein and other uses which are encompassed within the spirit of the disclosure, as defined by the scope of the claims, will be recognized by those skilled in the art.

A brief table of contents is provided below solely for the purpose of assisting the reader. Nothing in this table of contents is meant to limit the scope of the examples or disclosure of the application.

TABLE 6
Table of Contents For Example Section.

| Example # | Title | Brief Description |
|---|---|---|
| 1 | HTP Transformation of | Describes embodiments of the high |
| | Saccharopolyspora & Demonstration | throughput genetic engineering |
| | of SNP Library Creation | methods of the present disclosure. |
| 2 | HTP Genomic Engineering- | Describes approaches for |
| | Implementation of a SNP Library to | rehabilitating industrial organisms |
| | Rehabilitate/Improve an Industrial | through SNP swap methods of the |
| | Microbial Strain | present disclosure. |
| 3 | HTP Genomic Engineering- | Describes an implementation of SNP |
| | Implementation of a SNP Swap | swap techniques for improving the |
| | Library to Improve Strain Performance | performance of Saccharopolyspora |
| | in Spinosyns Production in | strain producing spinosyns. Also |
| | Saccharopolyspora. | discloses selected second and third |
| | | order mutation consolidations. |
| 4 | HTP Genomic Engineering- | Describes methods for improving the |
| | Implementation of a Promoter Swap | strain performance of host organisms |
| | Library to Improve an Industrial | through PRO swap genetic design |
| | Microbial Strain | libraries of the present disclosure. |
| 5 | HTP Genomic Engineering- | Describes an implementation of PRO |
| | Implementation of a PRO Swap | swap techniques for improving the |
| | Library to Improve Strain Performance | performance of Saccharopolyspora |
| | for Spinosyn Production | strain producing spinosyn. |
| 6 | Epistasis Mapping - An Algorithmic | Describes an embodiment of the |
| | Tool for Predicting Beneficial | automated tools/algorithms of the |
| | Mutation Consolidations | present disclosure for predicting |
| | | beneficial gene mutation |
| | | consolidations. |
| 7 | HTP Genomic Engineering - PRO | Describes and illustrates the ability of |
| | Swap Mutation Consolidation and | the HTP methods of the present |
| | Multi-Factor Combinatorial Testing | disclosure to effectively explore the |
| | | large solution space created by the |
| | | combinatorial consolidation of |
| | | multiple gene/genetic design |
| | | library combinations. |
| 8 | HTP Genomic Engineering- | Describes and illustrates an |
| | Implementation of a Terminator | application of the STOP swap |
| | Library to Improve an Industrial Host | genetic design libraries of the present |
| | Strain | disclosure. |
| 9 | HTP Genomic Engineering - Rapid | Describes methods for rapid |
| | Consolidation of Genetic Changes and for | consolidation of genetic changes and |
| | Generating Genetic Diversity in | for generating genetic diversity in |
| | Saccharopolyspora. | Saccharopolyspora strain |
| | | producing spinosyns. |
| 10 | HTP Genomic Engineering - Reporter | Describes embodiments of |
| | proteins and related assays for use in | utilization and quantitative |
| | Saccharopolyspora | evaluation of three reporter genes |
| | | in Saccharopolyspora spinosa |
| | | This invention also describes the |
| | | optimization and application of a |
| | | colorimetric assay that enables |
| | | quantitative evaluation of GusA |
| | | expression in S. spinosa. |
| 11 | HTP Genomic Engineering - Integrase | Describes an integrase-based |
| | based system for targeted and efficient | system for integration of genetic |
| | genomic integration in | elements into the genome of |
| | Saccharopolyspora spinosa | Saccharopolyspora |
| 12 | Origins of replication for self-replicating | Describes origins of replication and |
| | plasmid systems for Saccharopolyspora | replicative elements having replication |
| | spinosa. | function in Saccharopolyspora |
| 13 | HTP Genomic Engineering- | Describes an implementation of |
| | Implementation of a Terminator Library to | Ribosomal Binding Site techniques |
| | Improve an Industrial Host Strain | for improving the performance of |
| | | Saccharopolyspora strain producing |
| | | spinosyn. |
| 14 | HTP Genomic Engineering- | Describes an implementation of |
| | Implementation of a Transposon | transposon mutagenesis techniques for |
| | Mutagenesis Library to Improve Strain | improving the performance of |
| | Performance in Saccharopolyspora | Saccharopolyspora strain producing |
| | | spinosyns. |
| 15 | Neutral integration sites for the insertion | Describes an implementation of neutral |
| | of genetic elements in Saccharopolyspora | integration sites for integration of |
| | | sequences into the genome of |
| | | Saccharopolyspora |
| 16 | HTP Genomic Engineering- | Describes an implementation of |
| | Implementation of an Anti-metabolite | Anti-metabolite/Fermentation |
| | Selection/Fermentation Product Resistance | product Resistance techniques for |
| | Library to Improve Strain Performance in | improving the performance of |
| | Saccharopolyspora | Saccharopolyspora strain producing |
| | | spinosyn. |
| 17 | HTP Genomic Engineering - Use of sacB | Describes an implementation of methods |
| | or pheS as counterselection markers in S. spinosa | of creating scarless mutant |
| | for the generation of scarless | Saccharopolyspora strain using sacB or |
| | mutant strains | pheS as counterselection markers |
| 18 | HTP Conjugation of | Describes embodiments of the high |
| | Saccharopolyspora & Demonstration | throughput genetic engineering |
| | of Introducing Exogenous DNA into | methods of the present disclosure. |
| | Saccharopolyspora | |

### Example 1: HTP Transformation of Saccharopolyspora & Demonstration of SNP Library Creation

This example illustrates embodiments of the HTP genetic engineering methods of the present disclosure. Host cells are transformed with a variety of SNP sequences of different sizes, all targeting different areas of the genome. The results demonstrate that the methods of the present disclosure are able to generate rapid genetic changes of any kind, across the entire genome of a host cell.

### A. Cloning of Transformation Vectors

A variety of SNPs will be chosen at random from a predetermined Saccharopolyspora strain (e.g., a Saccharopolyspora spinose strain) and are cloned into Saccharopolyspora cloning vectors using yeast homologous recombination cloning techniques to assemble a vector in which each SNP was flanked by direct repeat regions, as described supra in the “Assembling/Cloning Custom Plasmids” section, and as illustrated in FIG. 3.

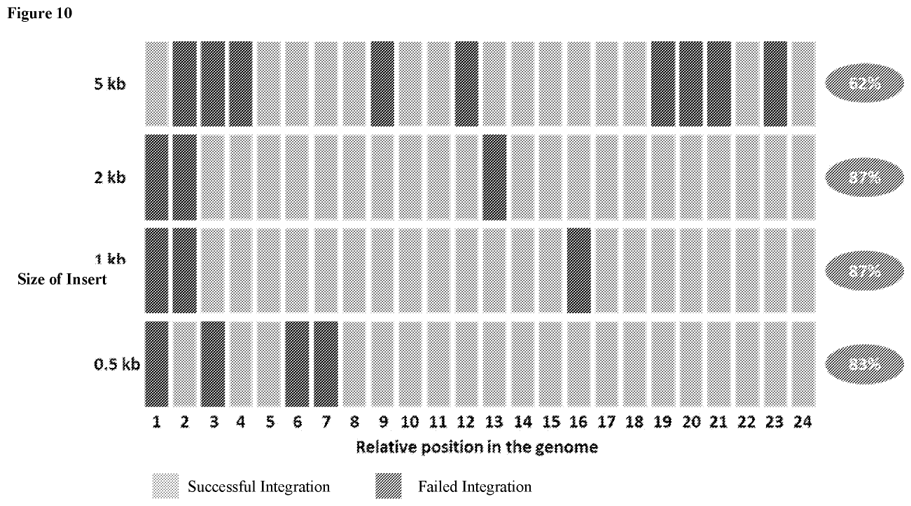

The SNP cassettes for this example will be designed to include a range of homology direct repeat arm lengths ranging from about 0.5 Kb, 1 Kb, 2 Kb, and 5 Kb, or any other desired lengths. Moreover, SNP cassettes will be designed for homologous recombination targeted to various distinct regions of the genome, as described in more detail below. See FIG. 10 for an exemplary transformation experiment demonstrated in Coynebacterium. However, similar procedures have been customized for Saccharopolyspora and are being successfully carried out by the inventors.

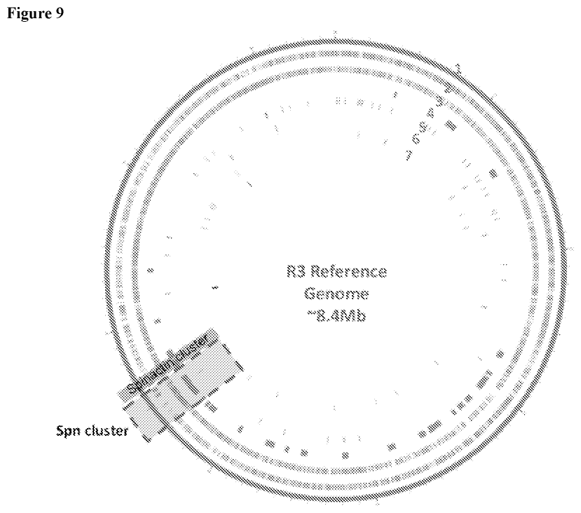

The S. spinosa genome is about 8,581,920 bp in size (see FIG. 9), and contains about 8,302 predicted coding sequences (CDSs), see Pan et al. (JOURNAL OF BACTERIOLOGY, June 2011, p. 3150-3151, doi:10.1128/JB.00344-11). The genome can be arbitrarily divided into equal-sized genetic regions, and SNP cassettes will be designed to target each of the regions.

Each DNA insert will be produced by PCR amplification of homologous regions using commercially sourced oligos and the host strain genomic DNA described above as template. The SNP to be introduced into the genome will be encoded in the oligo tails. PCR fragments will be assembled into the vector backbone using homologous recombination in yeast.

Cloning of each SNP and homology arm into the vector will be conducted according to the HTP engineering workflow described in FIG. 6A-B, FIG. 3, and Table 5.

B. Transformation of Assembled Clones into E. coli

Vectors will be initially transformed into E. coli using standard heat shock transformation techniques in order to identify correctly assembled clones, and to amplify vector DNA for Saccharopolyspora transformation.

For example, transformed E. coli bacteria will be tested for assembly success. Colonies from each E. coli transformation plate will be cultured and tested for correct assembly via PCR. This process will be repeated for each of the transformation locations and for each of the different insert sizes. Results from this experiment will be represented as the number of correct colonies identified out of the colonies that will be tested for each treatment (insert size and genomic location).

C. Transformation of Assembled Clones into Saccharopolyspora

Validated clones will be transformed into Saccharopolyspora spinosa host cells via electroporation. For each transformation, the number of Colony Forming Units (CFUs) per μg of DNA was determined as a function of the insert size. Genome integration will also be analyzed as a function of homology arm length.

Genomic integration efficiency will also be analyzed with respect to the targeted genome location in Saccharopolyspora spinosa transformants.

### D. Looping Out Selection Markers

Cultures of Saccharopolyspora identified as having successful integrations of the insert cassette will be cultured on media to counter select for loop outs of the selection gene. These results will illustrate whether loopout efficiencies remain steady across homology arm lengths of 0.5 kb to 5 kb, or other desired length.

In order to further validate loop out events, colonies exhibiting resistance will be cultured and analyzed via sequencing.

### Example 2: HTP Genomic Engineering—Implementation of a SNP Library to Rehabilitate/Improve an Industrial Microbial Strain

This example illustrates several aspects of the SNP swap libraries of the HTP strain improvement programs of the present disclosure. Specifically, the example illustrates several envisioned approaches for rehabilitating currently existing industrial strains. This example describes the wave up and wave down approaches to exploring the phenotypic solution space created by the multiple genetic differences that may be present between “base,” “intermediate,” and industrial strains.

### A. Identification of SNPs in Diversity Pool

An exemplary strain improvement program using the methods of the present disclosure will be conducted on an industrial production microbial strain, herein referred to as “C.” The diversity pool strains for this program are represented by A, B, and C. Strain A represented the original production host strain, prior to any mutagenesis. Strain C represented the current industrial strain, which has undergone many years of mutagenesis and selection via traditional strain improvement programs. Strain B represented a “middle ground” strain, which had undergone some mutagenesis, and had been the predecessor of strain C.

Strains A, B, and C are sequenced and their genomes will be analyzed for genetic differences between strains. All non-synonymous SNPs will be identified. Of these, certain SNPs will be unique to C, certain SNPs will be additionally shared by B and C, and certain SNPs will be unique to strain B. These SNPs will be used as the diversity pool for downstream strain improvement cycles.

### B. SNP Swapping Analysis

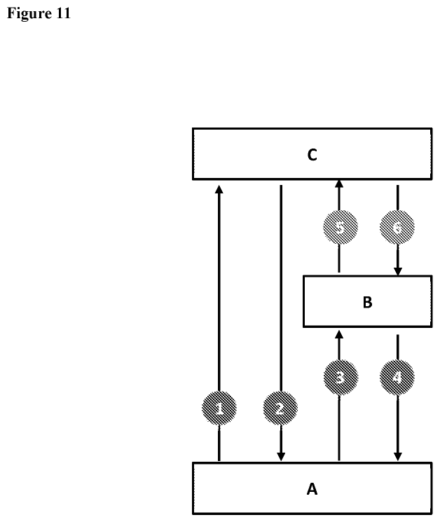

SNPs identified from the diversity pool in Part A of Example 2 will be analyzed to determine their effect on host cell performance. The initial “learning” round of the strain performance will be broken down into six steps as described below, and diagramed in FIG. 11.

First, all the SNPs from C will be individually and/or combinatorially cloned into the base A strain. The purpose of these transformants will be to identify beneficial SNPs.

Second, all the SNPs from C will be individually and/or combinatorially removed from the commercial strain C. The purpose of these transformants will be to identify neutral and detrimental SNPs. Additional optional steps 3-6 are also described below. The first and second steps of adding and subtracting SNPS from two genetic time points (base strain A, and industrial strain C) is herein referred to as “wave,” which comprises a “wave up” (addition of SNPs to a base strain, first step), and a “wave down” (removal of SNPs from the industrial strain, second step). The wave concept extends to further additions/subtractions of SNPS.

Third, all the SNPs from B will be individually and/or combinatorially cloned into the base A strain. The purpose of these transformants will be to identify beneficial SNPs. Several of the transformants will also serve as validation data for transformants produced in the first step.

Fourth, all the SNPs from B will be individually and/or combinatorially removed from the commercial strain B. The purpose of these transformants will be to identify neutral and detrimental SNPs. Several of the transformants will also serve as validation data for transformants produced in the second step.

Fifth, all the SNPs unique to C (i.e., not also present in B) will be individually and/or combinatorially cloned into the commercial B strain. The purpose of these transformants will be to identify beneficial SNPs. Several of the transformants will also serve as validation data for transformants produced in the first and third steps.

Sixth, all the SNPs unique to C will be individually and/or combinatorially removed from the commercial strain C. The purpose of these transformants will be to identify neutral and detrimental SNPs. Several of the transformants will also serve as validation data for transformants produced in the second and fourth steps.

Data collected from each of these steps is used to classify each SNP as prima facie beneficial, neutral, or detrimental.

Alternatively, in another example, Strain A represented the original production host strain, which may already has some, but not too many mutagenesis. Strain C represented the current industrial strain, which has undergone many years of mutagenesis and selection via traditional strain improvement programs. Strain B represented a “middle ground” strain, which is an old industrial strain having much less mutagenesis compared to strain C, but more mutagenesis compared to strain A. Similar steps as described above can be taken out to generate data and be used to classify each SNP. In some embodiments, instead of making all SNPs in each background strains, it is understood that certain set of SNPs can be chosen first and prioritized for further engineering.

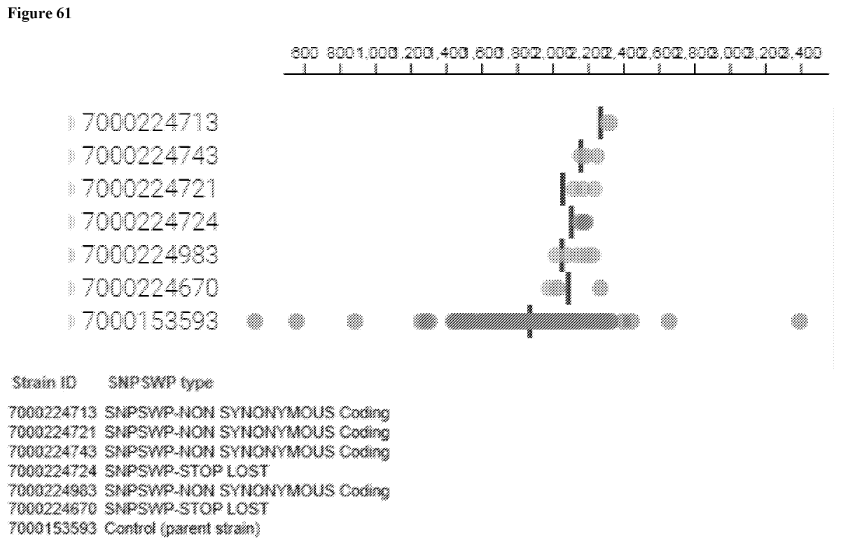

Data demonstrating the utility of this engineering approach is shown in FIG. 61. Mutagenic SNPs were identified in an advanced lineage strain by comparison to the base strain, and using the engineering approaches described above, these SNPs were scarlessly removed from the advanced strain. “SNPswap” strains were tested in comparison to the parent strain (advanced lineage strain) in a plate assay for polyketide productivity, and some strains exhibited an improvement compared to the parent strain.

### C. Utilization of Epistatic Mapping to Determine Beneficial SNP Combinations

Beneficial SNPs identified in Part B of Example 2 will be analyzed via the epistasis mapping methods of the present disclosure, in order to identify SNPs that are likely to improve host performance when combined.

New engineered strain variants will be created using the engineering methods of Example 1 to test SNP combinations according to epistasis mapping predictions. SNPs consolidation may take place sequentially, or may alternatively take place across multiple branches such that more than one improved strain may exist with a subset of beneficial SNPs. SNP consolidation will continue over multiple strain improvement rounds, until a final strain is produced containing the optimum combination of beneficial SNPs, without any of the neutral or detrimental SNP baggage

### Example 3: HTP Genomic Engineering—Implementation of a SNP Swap Library to Improve Strain Performance in Spinosyns Production in Saccharopolyspora

This example provides an illustrative implementation of a portion of the SNP Swap HTP design strain improvement program of Example 2 with the goal of producing yield and productivity improvements of spinosyns production in Saccharopolyspora spinosa.

Section B of this example further illustrates the mutation consolidation steps of the HTP strain improvement program of the present disclosure. The example thus provides experimental results for a first, second, and third round consolidation of the HTP strain improvement methods of the present disclosure.

Mutations for the second and third round consolidations are derived from separate genetic library swaps. These results thus also illustrate the ability for the HTP strain programs to be carried out multi-branch parallel tracks, and the “memory” of beneficial mutations that can be embedded into meta data associated with the various forms of the genetic design libraries of the present disclosure.

As described above, the genomes of a provided base reference strain (Strain A), and a second “engineered” strain (Strain C) were sequenced, and all genetic differences were identified. The base strain was a Saccharopolyspora spinosa variant that had not undergone mutagenesis. The engineered strain was also a Saccharopolyspora spinosa strain that had been produced from the base strain after several rounds of traditional mutation improvement programs.

A. HTP engineering and High Throughput Screening

Each of the identified SNPs will be individually added back into the base strain, according to the cloning and transformation methods of the present disclosure. Each newly created strain comprising a single SNP will be tested for spinosyns yield in small scale cultures designed to assess product titer performance. Small scale cultures will be conducted using media from industrial scale cultures. Product titer will be optically measured at carbon exhaustion (i.e., representative of single batch yield) with a standard colorimetric assay. Reactions will be allowed to proceed to an end point and optical density measured using a Tecan M1000 plate spectrophotometer.

B. Second Round HTP Engineering and High Throughput Screening—Consolidation of SNP Swap Library with Selected PRO Swap Hits

One of the strengths of the HTP methods of the present disclosure is their ability to store HTP genetic design libraries together with information associated with each SNP/Promoter/Terminator/Transposon mutagenesis/anti-metabolite/Start Codon's effects on host cell phenotypes. The present inventors had previously conducted a promoter swap experiment that had identified several promoter swaps in Saccharopolyspora spinosa (see e.g., Example 4).

The present inventors will modify the base strain A of this Example to also include one of the previously identified genetic diversity, such as those in the (1) Promoter swaps (PRO Swap) libraries, (2) SNP swaps libraries, (3) Start/Stop codon exchanges libraries, (4) STOP swaps libraries, (5) Sequence optimization libraries, (6) transposon mutagenesis diversity libraries, (7) ribosomal binding site (RBS) diversity libraries, and (8) anti-metabolite selection/fermentation product resistance libraries. The top genetic diversity identified from the initial screen will be re-introduced into this new base strain to create a new genetic diversity microbial library. As with the previous step, each newly created strain comprising one or more genetic diversities will be tested for spinosyn yield. Selected candidate strains will also tested for a productivity proxy, by measuring spinosyns production.

The results from this second round of SNP swap will identify SNPs capable of increasing base strain yield and productivity of spinosyns in a base strain comprising the promoter swap mutation.

### C. Tank Culture Validation

Strains containing top SNPs identified during the HTP steps above will be cultured into medium sized test fermentation tanks. Briefly, small cultures of each strain will be grown and used to inoculate large cultures in the test fermentation tanks with equal amounts of inoculate. The inoculate was normalized to contain the same cellular density.

The resulting tank cultures will be allowed to proceed for a determined time before harvest. Yield and productivity measurements will be calculated from substrate and product titers in samples taken from the tank at various points throughout the fermentation. Samples will be analyzed for particular small molecule concentrations by high pressure liquid chromatography using the appropriate standards.

### Example 4: HTP Genomic Engineering—Implementation of a Promoter Swap Library to Improve an Industrial Microbial Strain

Previous examples have demonstrated the power of the HTP strain improvement programs of the present disclosure for rehabilitating industrial strains. Examples 2 and 3 described the implementation of SNP swap techniques and libraries exploring the existing genetic diversity within various base, intermediate, and industrial strains

This example illustrates embodiments of the HTP strain improvement programs using the PRO swap techniques of the present disclosure. Unlike Example 3, this example teaches methods for the de-novo generation of mutations via PRO swap library generation.

### A. Identification of a Target for Promoter Swapping

As aforementioned, promoter swapping is a multi-step process that comprises a step of: Selecting a set of “n” genes to target.

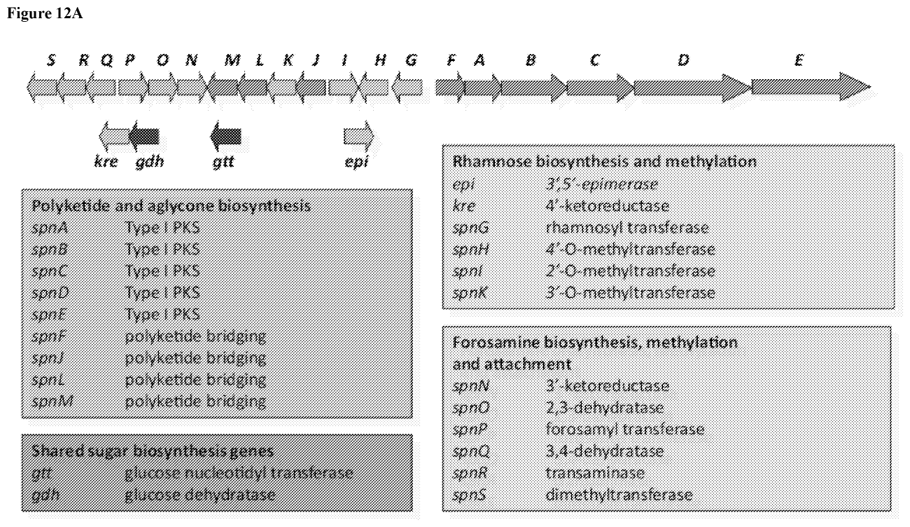

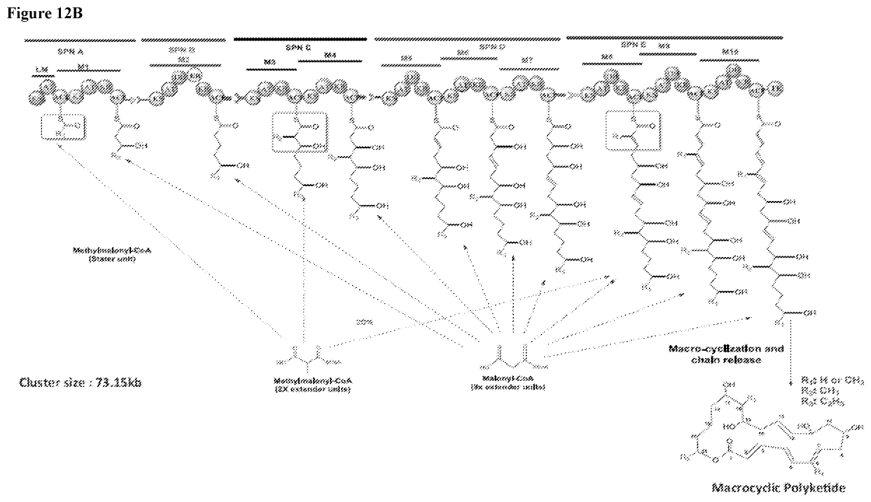

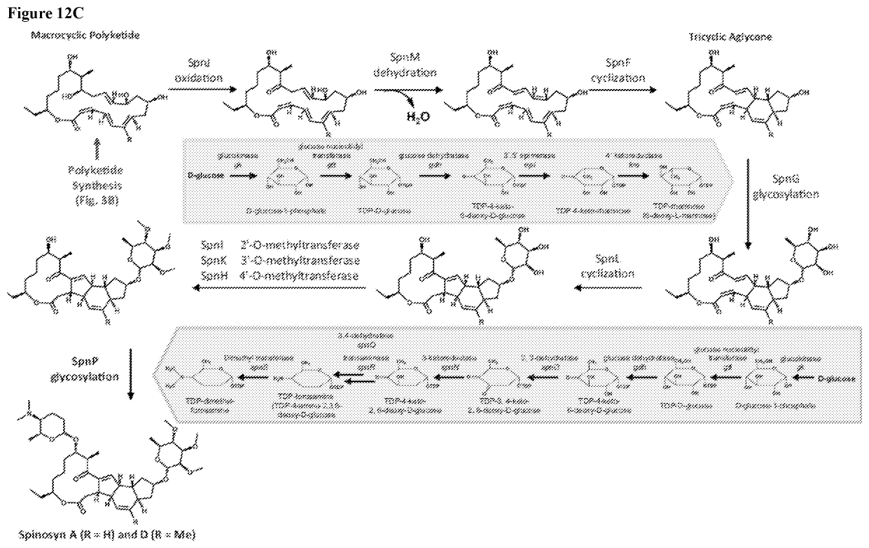

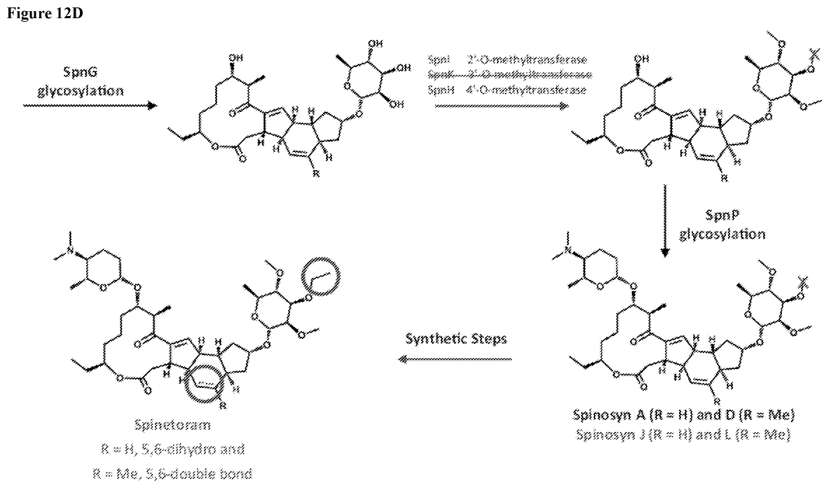

The method for genome engineering described here enables targeting any location in the genome for promoter swapping. In this example, the inventors have identified genes to modulate via the promoter ladder methods of the present disclosure, including core biosynthetic pathway genes listed below. (See, FIG. 12A to FIG. 12D). Additionally, genes related to precursor pools, cofactor availability, competing secondary metabolites, polyketide chaperones, key transcriptional regulators and sigma factors for secondary metabolite production, substrate and product transporters, as well as genes that have an unknown relationship to product formation (off-pathway genes) are all candidates for promoter swapping to enable strain improvement.

TABLE 7
Potential Genes involved in Spinosyn Production in S. spinosa

| Spinosyn Synthesis | |
|---|---|
| Pathway Genes | Gene information (Sequence, Function, etc.) |
| spnA | polyketide synthase loading and extender module |
| | 1 spnA |
| spnB | polyketide synthase extender module 2 spnB |
| spnC | polyketide synthase extender modules 3-4 spnC |
| spnD | polyketide synthase extender modules 5-7 spnD |
| spnE | polyketide synthase extender modules 8-10 spnE |
| spnF | methyltransferase-like protein spnF |
| spnG | putative NDP-rhamnosyltransferase spnG |
| spnH | putative O-methyltransferase spnH |
| spnI | putative O-methyltransferase spnI |
| spnJ | putative oxidoreductase spnJ |
| spnK | putative O-methyltransferase spnK |
| spnL | methyltransferase-like protein spnL |
| spnM | SpnM |
| spnN | putative NDP-hexose-3-ketoreductase spnN |
| spnO | putative NDP-hexose-2 3-dehydratase spnO |
| spnP | putative NDP-forosamyltransferase spnP |
| spnQ | putative NDP-hexose-3 4-dehydratase spnQ |
| spnR | putative aminotransferase spnR |
| spnS | putative N-dimethyltransferase spnS |
| kre | dTDP-4-dehydrorhamnose reductase kre |
| gdh | dTDP-glucose 4 6-dehydratase gdh |
| epi | dTDP-4-dehydrorhamnose 3 5-epimerase epi |
| gtt | Glucose-1-phosphate thymidylyltransferase 1 gtt |
| MetK | S-adenosylmethionine synthase MetK |
| PFK | Pyrophosphate--fructose 6-phosphate 1- |
| | phosphotransferase PFK |
| rsmG | Ribosomal RNA small subunit methyltransferase |
| | G rsmG |
| rpsL | 30S ribosomal protein S12 rpsL |
| gk | Glucokinase |
| asb1 | Anthranilate synthase component 1 asb1 |
| pntA | NAD(P) transhydrogenase subunit alpha part 1 |
| | pntA |
| pntB | NAD(P) transhydrogenase subunit alpha pntB |
| mmsd | Methylmalonate-semialdehyde dehydrogenase |
| | (acylating) |
| Acat | acetyl-CoA acetyltransferase |
| glcP | Glucopyranose |
| sucA | Oxoglutarate dehydrogenase E1 component |

### B. Creation of Promoter Ladder

Another step in the implementation of a promoter swap process is the selection of a set of “x” promoters to act as a “ladder”. Ideally these promoters have been shown to lead to highly variable expression across multiple genomic loci, but the only requirement is that they perturb gene expression in some way.

These promoter ladders, in particular embodiments, are created by: identifying natural, native, or wild-type promoters associated with the target gene of interest and then mutating said promoter to derive multiple mutated promoter sequences. Each of these mutated promoters is tested for effect on target gene expression. In some embodiments, the edited promoters are tested for expression activity across a variety of conditions, such that each promoter variant's activity is documented/characterized/annotated and stored in a database. The resulting edited promoter variants are subsequently organized into “ladders” arranged based on the strength of their expression (e.g., with highly expressing variants near the top, and attenuated expression near the bottom, therefore leading to the term “ladder”).

In the present exemplary embodiment, the inventors will create promoter ladder: ORF combinations for each of the target genes in the spinosyn synthesis pathway.

A major goal of our genetic engineering efforts, and metabolic engineering more broadly, is to alter host metabolism, optimize biosynthetic pathways, and introduce or duplicate pathway genes in order to improve the yield of a desired product. Success relies on the ability to perturb and balance expression of genes both within (on-pathway) and outside (off-pathway) of the biosynthetic gene cluster or over-express non-native genes or copies of genes that are introduced. This invention is a genetic tool which allows us to perturb and tune gene expression in S. spinosa.

Multiple rounds of engineering are often required for engineering improved phenotypes. The genetic diversity of this ladder circumvents engineering challenges associated with using repeated DNA sequences (e.g. homology regions for off-target recombination) and for transcriptional dilution effects. Because of the sequence and source diversity of sequences in this ladder, this invention circumvents these challenges.

Promoter ladders exist for other, more common hosts (model organisms; for examples see Siegl et al. (2013, “Design, construction and characterization of a synthetic promoter library for fine-tuned gene expression in actinomycetes.” Metab Eng. 19:98-106) and Seghezzi et al. (2011, “The construction of a library of synthetic promoters revealed some specific features of strong Streptomyces promoters.” Appl Microbiol Biotechnol. 90(2):615-23), but S. spinosa is a largely intractable host and few genetic tools have been developed for this organism. This invention represents the first promoter ladder developed and quantitatively characterized in S. spinosa. Additionally, we anticipate that promoters described here will show predictable dynamics in nearby hosts.

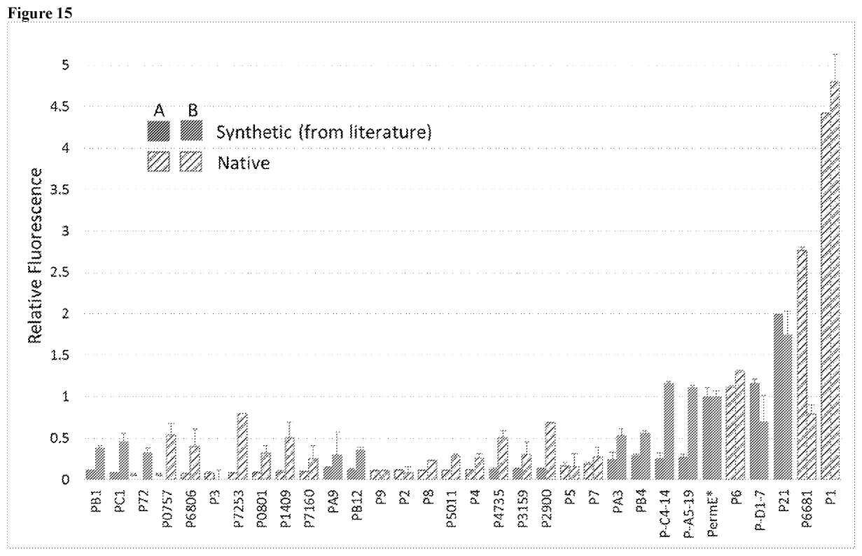

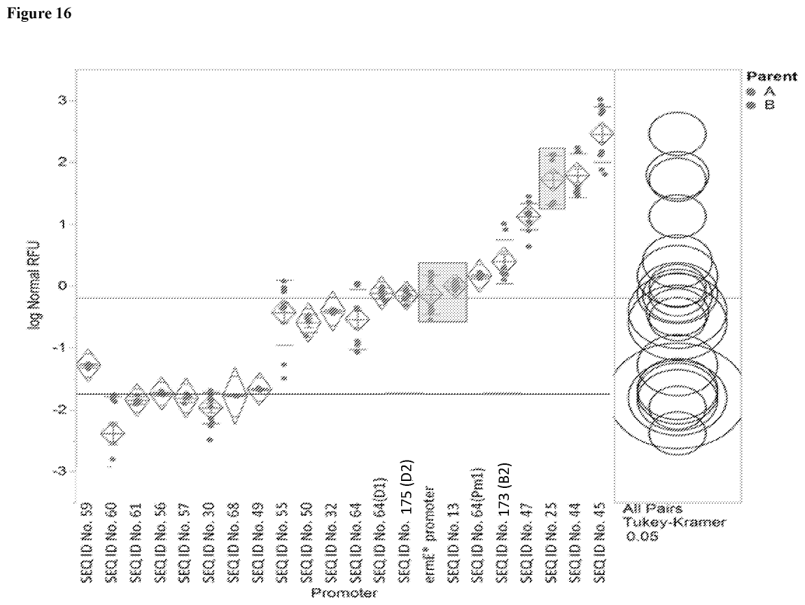

The approach that we employed to identify and select putative native promoter sequences made use of data available to us. An assembled and annotated reference genome for S. spinosa was used to identify intergenic regions upstream of the predicted coding sequences of genes. RNAseq data (a replicated time series sampled during fermentation and comparing expression in two strains) was used to identify strongly expressed genes and genes with different temporal expression profiles. Sequences upstream of genes of interest (GOIs) were then selected for construction of the promoter-fluorescent protein expression cassette. Promoter strength was assessed indirectly by quantifying and comparing relative GFP fluorescence in promoter ladder strains grown under fermentation-relevant seed culture conditions and production culture conditions. Potentially useful promoters are listed in the Table 8 below. The first round of promoter evaluation resulted in a ladder of promoter strengths (FIG. 15). Through subsequent evaluation we were able to identify additional functional promoters, including some that were significantly stronger than those originally identified (FIG. 16).

TABLE 8
Summary of the promoter ladder: sequence names,
sources, characteristics and test status

| SEQ ID | Name | Assoc. Gene | bp | Type | Status |
|---|---|---|---|---|---|
| 1 | P7160 | chaperonin GroEL | 141 | native | tested |
| 2 | P7253 | Elongation factor Tu | 198 | native | tested |
| 3 | P6681 | F-type ATPase subunit delta | 232 | native | tested |
| 4 | P6316 | PspA/IM30 family protein | 361 | native | tested |
| 5 | P6806 | 2-oxoglutarate decarboxylase | 303 | native | tested |
| 6 | P3159 | putative enoyl-CoA hydratase echA8 | 253 | native | tested |
| 7 | P0757 | putative L-lysine-epsilon aminotransferase | 168 | native | tested |
| 8 | P5011 | hypothetical protein | 363 | native | tested |
| 9 | P1409 | NAD-specific glutamate dehydrogenase | 300 | native | tested |
| 10 | P4735 | leucyl aminopeptidase (aminopeptidase T) | 322 | native | tested |
| 11 | P2900 | Cytochrome P450-terp | 149 | native | tested |
| 12 | P0801 | Periplasmic murein peptide-binding protein precursor | 199 | native | tested |
| 13 | P21 | — | 41 | Siegl et al. | tested |
| 14 | PA9 | — | 41 | Siegl et al. | tested |
| 15 | PA3 | — | 41 | Siegl et al. | tested |
| 16 | PB4 | — | 41 | Siegl et al. | tested |
| 17 | PB12 | — | 41 | Siegl et al. | tested |
| 18 | PB1 | — | 41 | Siegl et al. | tested |
| 19 | PC1 | — | 41 | Siegl et al. | tested |
| 20 | P72 | — | 41 | Siegl et al. | tested |
| 21 | P-C4-1 | — | 44 | Seghezzi et al. | tested |
| 22 | P-A5-19 | — | 44 | Seghezzi et al. | tested |
| 23 | P-C4-14 | — | 44 | Seghezzi et al. | tested |
| 24 | P-D1-7 | — | 44 | Seghezzi et al. | tested |
| 25 | P1 | secreted protein | 242 | native | tested |
| 26 | P2 | hypothetical protein | 65 | native | tested |
| 27 | P3 | RNA polymerase sigma factor SigD | 201 | native | tested |
| 28 | P3v2 | RNA polymerase sigma factor SigD | 300 | native | |
| 29 | P4 | Antigen Ag88 | 220 | native | tested |
| 30 | P4v2 | Antigen Ag88 | 177 | native | |
| 31 | P5 | DNA-directed RNA polymerase subunit beta | 200 | native | tested |
| 32 | P5v2 | DNA-directed RNA polymerase subunit beta | 299 | native | |
| 33 | P6 | molecular chaperone GroEL | 240 | native | tested |
| 34 | P7 | UDP-4-amino-4-deoxy-L-arabinose--oxoglutarate | 242 | native | tested |
| | | aminotransferase | | | |
| 35 | P8 | Proline racemase | 300 | native | tested |
| 36 | P9 | Phenyloxazoline synthase MbtB | 359 | native | tested |
| 37 | PspnA | polyketide synthase loading and extender module 1 | 433 | native | tested |
| 38 | PspnA_v2 | polyketide synthase loading and extender module 1 | 115 | native | tested |
| 39 | PspnF | methyltransferase-like protein | 496 | native | tested |
| 40 | PspnG | putative NDP-rhamnosyltransferase | 202 | native | tested |
| 41 | PspnQ | putative NDP-hexose-3 | 379 | native | tested |
| 42 | PspnQ_v2 | putative NDP-hexose-3 | 261 | native | tested |
| 49 | P1765 | Glutamine synthetase 1 | 332 | native | tested |
| 50 | P3747 | hypothetical protein | 300 | native | tested |
| 51 | P5078 | hypothetical protein | 247 | native | tested |
| 52 | P7419 | anaerobic benzoate catabolism transcriptional regulator | 230 | native | tested |
| 53 | P7156 | RNA polymerase sigma factor SigD | 300 | native | tested |
| 54 | P7256 | 30S ribosomal protein S12 | 300 | native | tested |
| 55 | P1941 | Response regulator protein vraR | 298 | native | tested |
| 56 | P3405 (P8) | Proline racemase | 300 | native | tested |
| 57 | P3407 | ABC transporter arginine-binding protein 1 precursor | 300 | native | tested |
| 58 | P2428 | acetyl-CoA synthetase | 248 | native | tested |
| 59 | P0927 | 4-hydroxyphenylpyruvate dioxygenase | 250 | native | tested |
| 60 | P0889 | Linear gramicidin dehydrogenase LgrE | 250 | native | tested |
| 61 | P0186 | L,D-transpeptidase catalytic domain | 298 | native | tested |
| 62 | P3702_v2 | hypothetical protein | 296 | native | tested |
| 63 | P7156_v2 | RNA polymerase sigma factor SigD | 300 | native | tested |
| 64 | P7256_v2 | 30S ribosomal protein S12 | 226 | native | tested |
| 65 | P1765_v2 | Glutamine synthetase 1 | 191 | native | tested |
| 66 | P7539_v2 | Antigen Ag88 | 177 | native | tested |
| 67 | P7276_v2 | DNA-directed RNA polymerase subunit beta | 299 | native | tested |
| 68 | P0941_v2 | hypothetical protein | 266 | native | tested |
| 69 | P0889_v2 | Linear gramicidin dehydrogenase LgrE | 163 | native | tested |
| 43 | P21_mutant | — | 41 | synth | tested |
| 44 | P1_core | secreted protein | 40 | native | tested |
| 45 | P1(−33) | secreted protein | 209 | native | tested |
| 46 | P1 + ribswtch | secreted protein | 433 | native | tested |
| 47 | P21-P1 | — | 53 | synth | tested |
| 48 | P1-P21 | — | 52 | synth | tested |
| 172 | Pmut-1 | — | 41 | synth | tested |
| 173 | B2 | — | 41 | synth | tested |
| 174 | D1 | — | 41 | synth | tested |
| 175 | D2 | — | 41 | synth | tested |

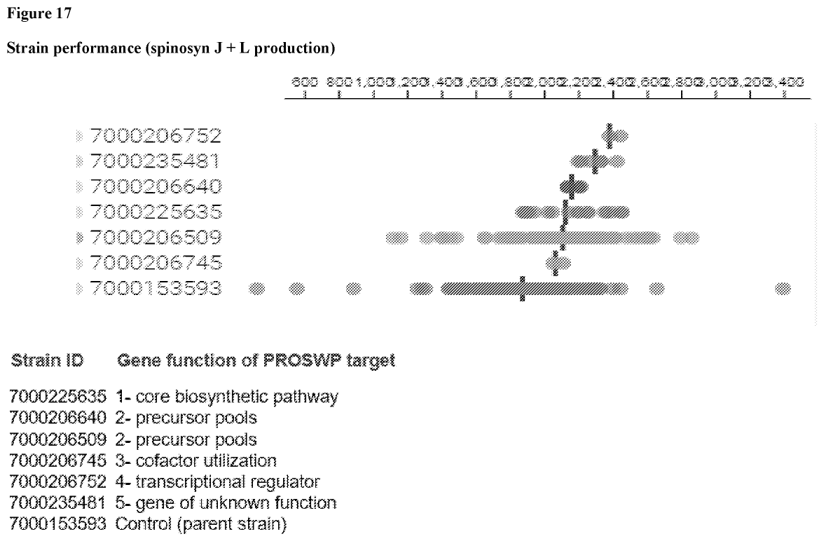

Expression strength of promoters in the library was characterized by using a fluorescent reporter protein placed downstream of the promoter sequence. Promoter-reporter sequences were integrated into a neutral integration site in the genome of two distinct experimental strains and fluorescence was measured under different growth regimes to provide a quantitative metric for promoter strength. This promoter library allows for modulation of gene expression (increase, decrease or alter temporal dynamics) in S. spinosa and related hosts and for engineering improved phenotypes. This invention has several applications for the genetic engineering of this host: 1) for use with PROSWP (Zymergen technology); 2) for overexpression of heterologous or duplicated copies of native genes; 3) for balancing expression of multi-gene integrations of biosynthetic or related genes. Engineering select promoter-gene pairs results in improved spinosyn production in certain strains (FIG. 17).

Thus, the inventors at least provide the following promoters to form the promoter library:

(1) Native promoter sequences newly identified from the S. spinosa genome;
(2) Synthetic promoter sequences (Siegl et al. and Seghezzi et al) described for use in related host organisms;
(3) A mutagenesis library of diverse promoter sequences; and
(4) Hybrid promoter sequences consisting of combinatorial re-arrangements of promoters (in progress).

These promoters show a range of expression strengths while consisting of significant nucleotide diversity (see FIG. 15 and FIG. 16). This library of promoters provide a set of DNA sequences that regulate expression of downstream genes, which can be used in S. spinosa and related hosts. The library described here exhibits a “ladder” of expression strengths, e.g., which span about 50 to 100 folds dynamic range (See FIG. 15 and FIG. 16), and additionally shows a range of nucleotide diversity. Together this library of promoters can be used in combination for precise tuning of a host genome for iterative rounds of engineering to improve any measurable phenotype. Each promoter type, strength and unique sequence provides an opportunity to overcome unknowns and challenges often faced in metabolic engineering. Such changes include, but are not limited to: (1) the inability to accurately predict how a promoter will function in each unique context (how it will effect expression of a given gene); (2) the level of expression that will be optimal for given gene; (3) the inability to predict how temporal dynamics or regulation the success of a perturbation; and (4) the levels of expression that will result in a balanced or optimized biosynthetic pathway. The promoters described herein can interact with specific gene targets to confer strain genotypes for improved production of chemicals in S. spinosa, such as spinosyns.

C. Associating Promoters from the Ladder with Target Genes

Another step in the implementation of a promoter swap process is the HTP engineering of various strains that comprise a given promoter from the promoter ladder associated with a particular target gene.

If a native promoter exists in front of target gene n and its sequence is known, then replacement of the native promoter with each of the x promoters in the ladder can be carried out. When the native promoter does not exist or its sequence is unknown, then insertion of each of the x promoters in the ladder in front of gene n can be carried out. In this way a library of strains is constructed, wherein each member of the library is an instance of x promoter operably linked to n target, in an otherwise identical genetic context (see e.g., FIG. 13).

### D. HTP Screening of the Strains

A final step in the promoter swap process is the HTP screening of the strains in the aforementioned library. Each of the derived strains represents an instance of x promoter linked to n target, in an otherwise identical genetic background.

By implementing a HTP screening of each strain, in a scenario where their performance against one or more metrics is characterized, the inventors are able to determine what promoter/target gene association is most beneficial for a given metric (e.g. optimization of production of a molecule of interest). See, FIG. 13.

In the exemplary embodiment illustrated in FIG. 17, the inventors have utilized the promoter swap process to optimize the production of spinosyn. An application of the Pro SWAP methods described above is described in Example 5, below.

### Example 5: HTP Genomic Engineering—Implementation of a PRO Swap Library to Improve Strain Performance for Spinosyn Production

The section below provides an illustrative implementation of the PRO swap HTP design strain improvement program tools of the present disclosure, as described in Example 4. In this example, a S. spinosa strain was subjected to the PRO swap methods of the present disclosure in order to increase host cell yield of spinosyns.

### A. Promoter Swap

Promoter Swaps were conducted as described in Example 4. Genes across the genome hypothesized to play a role in spinosyn production were targeted for promoter swaps using the promoter ladder listed (e.g. FIG. 13). Such genes for promoter Swaps include, but are not limited to: (1) genes in core biosynthetic pathway of a compound of interest, such as a spinosyn; (2) genes involved in precursor pool availability of a compound of interest, such as a gene directly involved in precursor synthesis or regulation of pool availability; (3) genes involved in cofactor utilization; (4) genes encoding with transcriptional regulators; (5) genes encoding transporters of nutrient availability; and (6) product exporters, etc.

### B. HTP Engineering and High Throughput Screening

HTP engineering of the promoter swaps was conducted as described in Example 1 and 3. HTP screening of the resulting promoter swap strains was conducted as described in Example 3. A number of genes across different functional dimensions (ranging from the core biosynthetic cluster to off-pathway) were targeted for promoter swap, and data showing improved strain performance compared to the parent strain is presented in FIG. 17.

Similarly, Promoter Swaps will be conducted for selected genes from the spinosyn biosynthetic pathway described on the left panel of FIG. 13, and genes across the genome to identify new improved strains, which will be targeted for promoter swaps using the promoters described in Table 8 above.

When visualized, the results of the promoter swap library screening will serve to identify gene targets that are most closely correlated with the performance metric being measured.

Selected strains will be re-cultured in small plates and tested for spinosyn yield as describe above.

### Example 6: Epistasis Mapping—an Algorithmic Tool for Predicting Beneficial Mutation Consolidations

This example describes an embodiment of the predictive modeling techniques utilized as part of the HTP strain improvement program of the present disclosure. After an initial identification of potentially beneficial mutations (through the use of genetic design libraries as described above), the present disclosure teaches methods of consolidating beneficial mutations in second, third, fourth, and additional subsequent rounds of HTP strain improvement. In some embodiments, the present disclosure teaches that mutation consolidations may be based on the individual performance of each of said mutations. In other embodiments, the present disclosure teaches methods for predicting the likelihood that two or more mutations will exhibit additive or synergistic effects if consolidated into a single host cell. The example below illustrates an embodiment of the predicting tools of the present disclosure.

Selected mutations from the SNP swap and promoter swapping (PRO swap) libraries of Examples 3 and 5 will be analyzed to identify SNP/PRO swap combinations that would be most likely to lead to strain host performance improvements.

SNP swapping library sequences will be compared to each other using a cosine similarity matrix, as described in the “Epistasis Mapping” section of the present disclosure. The results of the analysis will yield functional similarity scores for each SNP/PRO swap combination. A visual representation of the functional similarities among all SNPs/PRO swaps is depicted in a heat map in FIG. 53. The resulting functional similarity scores will also be used to develop a dendrogram depicting the similarity distance between each of the SNPs/PRO swaps, similar to the example in FIG. 54A.

Mutations from the same or similar functional group (i.e., SNPs/PRO swaps with high functional similarity) are more likely to operate by the same mechanism, and are thus more likely to exhibit negative or neutral epistasis on overall host performance when combined. In contrast, mutations from different functional groups would be more likely to operate by independent mechanisms, and thus more likely to produce beneficial additive or combinatorial effects on host performance.

In order to illustrate the effects of biological pathways on epistasis, SNPs and PRO swaps exhibiting various functional similarities will be combined and tested on host strains. Three SNP/PRO swap combinations will be engineered into the genome of S. spinosa as described in Example 1.

The performance of each of the host cells containing the SNP/PRO swap combinations will be tested as described in Example 3, and will compared to that of a control host cell.

Thus, the epistatic mapping procedure is useful for predicting/programming/informing effective and/or positive consolidations of designed genetic changes. The analytical insight from the epistatic mapping procedure allows for the creation of predictive rule sets that can guide subsequent rounds of microbial strain development. The predictive insight gained from the epistatic library may be used across microbial types and target molecule types.

### Example 7: HTP Genomic Engineering—Pro Swap Mutation Consolidation and Multi-Factor Combinatorial Testing

Previous examples have illustrated methods for consolidating a small number of pre-selected PRO swap mutations with SNP swap libraries (Example 3). Other examples have illustrated the epistatic methods for selecting mutation consolidations that are most likely to yield additive or synergistic beneficial host cell properties (Example 6). This example illustrates the ability of the HTP methods of the present disclosure to effectively explore the large solution space created by the combinatorial consolidation of multiple gene/genetic design library combinations (e.g., PRO swap library x SNP Library or combinations within a PRO swap library).

In this illustrative application of the HTP strain improvement methods of the present disclosure, promoter swaps identified as having a positive effect on host performance in Example 5 will be consolidated in second order combinations with the original PRO swap library. The decision to consolidate PRO swap mutations is based on each mutation's overall effect on yield or productivity, and the likelihood that the combination of the two mutations would produce an additive or synergistic effect.

### A. Consolidation Round for PRO Swap Strain Engineering

Strains will be transformed as described in previous Example 1. Briefly, strains already containing one desired PRO swap mutation will be once again transformed with the second desired PRO swap mutation.

The HTP methods for exploring solution space of single and double consolidated mutations, can also be applied to third, fourth, and subsequent mutation consolidations.

### Example 8: HTP Genomic Engineering—Implementation of a Terminator Library to Improve an Industrial Host Strain

The present example applies the HTP methods of the present disclosure to additional HTP genetic design libraries, including STOP swap. The example further illustrates the ability of the present disclosure to combine elements from basic genetic design libraries (e.g., PRO swap, SNP swap, STOP swap, etc.) to create more complex genetic design libraries (e.g., PRO-STOP swap libraries, incorporating both a promoter and a terminator). In some embodiments, the present disclosure teaches any and all possible genetic design libraries, including those derived from combining any of the previously disclosed genetic design libraries.

In this example, a small scale experiment will be conducted to demonstrate the effect of the STOP swap methods of the present invention on gene expression. Terminators of the present disclosure will be paired with one of two native S. spinosa promoters as described below, and will be analyzed for their ability to impact expression of a fluorescent protein.

Combinatorial genetic engineering and metabolic pathway refactoring approaches rely on libraries of DNA elements (e.g., promoters, ribosomal binding sites, transcription terminators) that can be employed in combination or inserted into the host genome at precise locations in order to perturb gene expression and affect production of a target molecule or alter a desired host phenotype. An improved understanding, and the quantitative assessment/characterization, of these libraries is desirable as it offers an opportunity to improve predictability of genetic changes. Of the common DNA library types, transcription terminators, are arguably the least understood. Terminators play a role in (1) completing transcription, but they also (2) influence mRNA half-life (Curran et al., 2015, “Short Synthetic Terminators for Improved Heterologous Gene Expression in Yeast.” ACS Synth. Biol. 4(7): 824-832), and in turn protein expression. Accordingly, terminators should be considered an important component of any synthetic biology toolkit. The creation of terminator libraries, or ladders, requires a mechanism to assess and quantify terminator performance based on both of two criteria: (1) ability to terminate transcription; (2) ability to influence mRNA half-life and expression of the upstream gene. The present disclosure provides a robust, and first, tool with which to do this in S. spinosa.

Similar solutions exist and have been employed in other organisms (Chen et al., 2013, “Characterization of 582 natural and synthetic terminators and quantification of their design constraints.” Nat. Methods 10, 659-664, and Cambray et al., 2013, “Measurement and modeling of intrinsic transcription terminators.” Nucleic Acids Research. 41(9): 5139-5148), but the present disclosure provides the first (1) system and assay for assessing terminator functionality; (2) transcription terminator library that has been developed and characterized, in S. spinosa.

To identify putative terminators, genomic sequence from S. spinosa and S. erythraea were entered into an online tool for the prediction of rho-independent terminators in nucleic acid sequences). Twelve terminator sequences (four native and eight heterologous sequences see Table 9 below) predicted by the online tool that occurred downstream (in intergenic regions) of well-annotated genes were selected for analysis.

TABLE 9
Sequences, sources and size
of putative terminators tested.

| | Associated | | | Size |
|---|---|---|---|---|
| ID | Gene | Sequence | Source | (bp) |
| T1 | (elongation | CCCGAACCTTCGGGG | S. | 37 |
| | factor | GCGGGCCCTCTTGCT | spinosa | |
| | tu) | TTTCAAT | | |
| | | (SEQ ID No. 70) | | |
| T2 | (Leucyl | CGGGCAATAATACGT | S. | 49 |
| | amino- | GCCCGGACGGTAGTG | spinosa | |
| | peptidase) | CGAGCACGAGGTGGG | | |
| | | TACG | | |
| | | (SEQ ID No. 71) | | |
| T3 | (cytochrome | AGTTTGTCGAACCGG | S. | 41 |
| | P450 | CGGCGTTCGCCGGcT | spinosa | |
| | hydroxylase) | TTACCTTGCGC | | |
| | | (SEQ ID No. 72) | | |
| T4 | (F0F1 ATP | GGTTTCTCGAACCAG | S. | 42 |
| | synthase | TGCTTTGCGTACTGG | spinosa | |
| | subunit | TTGTCGTTGCAG | | |
| | beta) | (SEQ ID No. 73) | | |
| T5 | (FAD-linked | CGGAGCCAGAGGGCG | S. | 37 |
| | oxido- | CCTGAGTGCCTGTTT | erythraea | |
| | reductase) | TTGATCC | | |
| | | (SEQ ID No. 74) | | |
| T6 | (phospho- | AAACGCCCCCGGCTC | S. | 39 |
| | ribosyl- | CGGCCGGGGGCgTTT | erythraea | |
| | transferase) | TTGGTTGTG | | |
| | | (SEQ ID No. 75) | | |
| T7 | (ATP-binding | AGACGCAGGAGGTCT | S. | 37 |
| | protein) | CGTGAGGGGCTTTTC | erythraea | |
| | | CGCGAGC | | |
| | | (SEQ ID No. 76) | | |
| T8 | (50 s | CGTGTGACTTGTCCC | S. | 35 |
| | Ribosomal | ACTCGGGGTTTTTGT | erythraea | |
| | protein L32) | CGCGA | | |
| | | (SEQ ID No. 77) | | |
| T9 | (tRNA-Arg) | GGATTCGTCCGGCCG | S. | 39 |
| | | AGGCCAATCGGCTTT | erythraea | |
| | | TCGGGGCCC | | |
| | | (SEQ ID No. 78) | | |
| T11 | (lsr2) | GCTTTCGTCGGCCGG | S. | 38 |
| | | GAACGCCCTGGTGTT | erythraea | |
| | | TCTTACCG | | |
| | | (SEQ ID No. 79) | | |
| T12 | (AraC) | TTGGGTGGATTCACC | S. | 38 |
| | | CCTACCGGGTGTTTT | erythraea | |
| | | TCTCGGCT | | |
| | | (SEQ ID No. 80) | | |
| NoT | none | — | — | 0 |

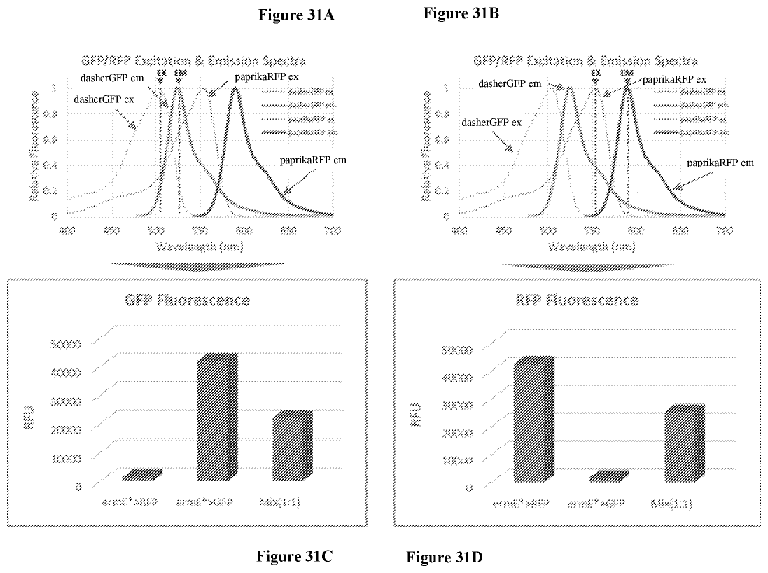

To test these putative terminators, a dual, reporter design and assay was utilized. The dual, reporter design and assay used in the test (which is further described in Example 10) enables the rapid assessment of functionality and relative strength of putative transcription terminator sequences. The assay uses two fluorescent reporter proteins (dasherGFP and paprikaRFP; IP-free sequences from DNA2.0) with distinct spectral signatures (FIG. 31A-D) to assess the performance of putative transcription terminators. The system enables the user to assess a putative terminator for its ability to 1) stop transcription, and 2) influence expression of the upstream gene. The dual, fluorescent reporter test cassette enables the quantitative assessment of strength, and a mechanisms by which to evaluate the influence on mRNA stability, of putative terminator sequences required for genetic engineering of S. spinosa.

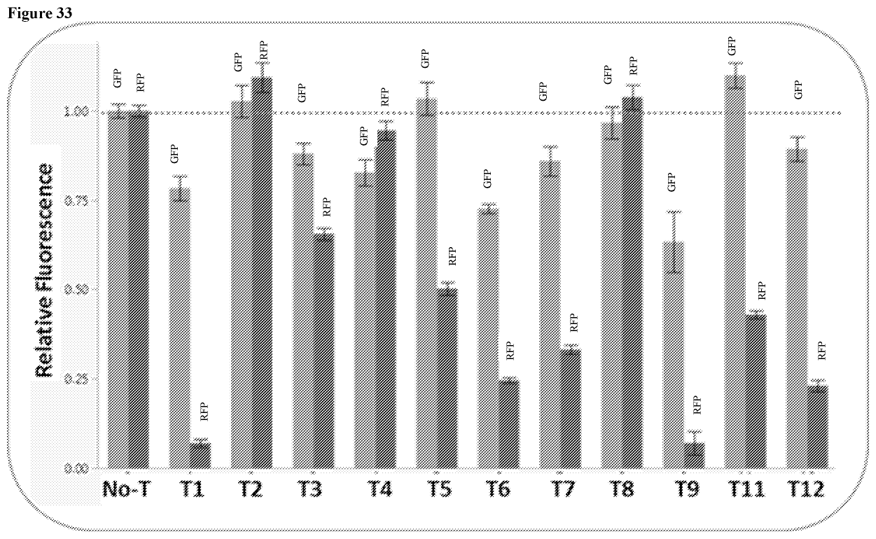

The quantitative assessment of these performance criteria is enabled by the design which utilizes bi-cistronic expression of the two fluorescent proteins driven by the ermE* promoter (Bibb et al., 1985, “Cloning and analysis of the promoter region of the erythromycin resistance gene (ermE) of Streptomyces erythraeus.” Gene. 38 (1-3):215-226). Each putative terminator sequence was cloned between the two reporters (downstream of GFP and upstream of an RBS and RFP). Expression (fluorescence) of the downstream reporter (RFP) was determined relative to that of the upstream reporter (GFP) after normalization using the GFP and RFP fluorescence of a positive control (the same polycistronic cassette without a terminator sequence between the reporters; NoT; see, FIG. 33). This system provides a robust mechanism for the quantitative assessment of terminator libraries and has utility for identifying and characterizing performance of putative terminator sequences for use in genetic engineering of S. spinosa. The strength of the system lies in application of two fluorescent reporters with distinct fluorescent spectra (FIG. 31A-D). The reporters allow quantification of fluorescence (protein expression of each reporter) across a large dynamic range (˜50×) without spectral interference from the other reporter and therefore eliminate the need for complicated signal correction (disentanglement of overlapping fluorescence signals). The expression of each reporter can be measured independently. These values then allow one to assess the performance of the genetic elements that contribute to each reporter's expression by comparing fluorescence (RFU) relative to the other reporter and the fluorescence resulting from the control strain without the terminator. By keeping all other elements constant while swapping the putative terminator sequence between the two reporters, one is able to indirectly evaluate: (1) the impact of a terminator on mRNA stability, by comparing the relative fluorescence of the upstream reporter (GFP), when different terminators are present; (2) the ability of a terminate to stop transcription, by comparing the relative fluorescence of the downstream reporter (RFP) to that of the upstream reporter (GFP) after normalization by fluorescence of the control strain without the terminator. This system allow us to identify (1) functional terminators and (2) terminators that differ in their ability to influence, or have characteristics that promote, mRNA stability.

Nucleic acid sequences of candidate terminators were cloned into the test cassette and integrated into the S. spinosa genome at a known neutral integration site. Resulting strains were grown in liquid culture (seed media) for 48 hours, washed with PBS and fluorescence (GFP and RFP) was measured using a plate reader. Fluorescence was normalized to absorbance at OD₅₄₀.

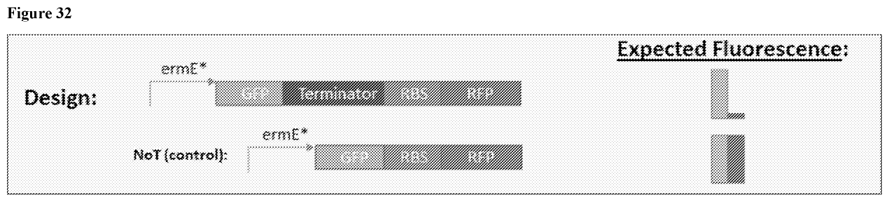

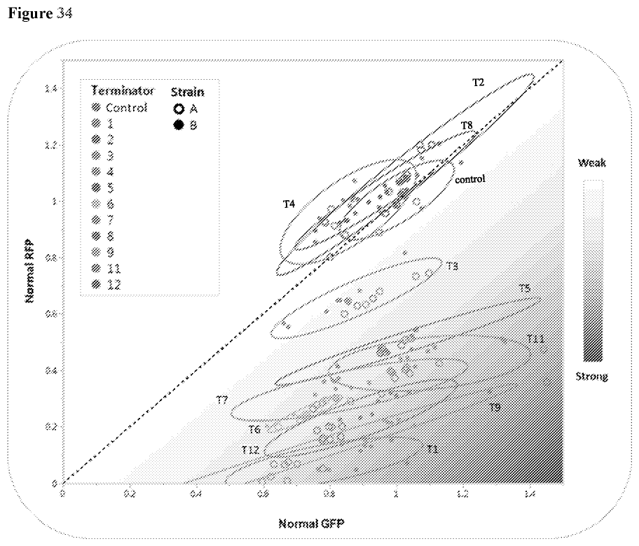

Based on the assay, a library of eleven transcription terminator sequences (four native and seven heterologous sequences—from S. erythraea) with a range of functionality or strengths (ability to cease transcription to downstream genes or attenuate transcription of upstream genes) in S. spinosa. These sequences range from 35-49 nucleotides in length and can easily be incorporated into engineering designs (Table 3; Table 8; FIG. 32 and FIG. 33). The result is a diverse library of terminators that vary in their strengths and influence on mRNA stability that offers the engineer a larger and more diverse solution space and opportunity to perturb and manipulate expression of target genes (FIG. 34).

The library of transcription terminator sequences of the present disclosure provides a tool required for the genetic engineering of S. spinosa. Transcription terminator sequences have several engineering applications: (1) as insulators of promoters or gene integrations, to protect against unintended consequences of upstream regulation; (2) as transcription terminators for gene insertions; and (3) for tuning expression and balancing pathways through their influence on mRNA stability or by insertion upstream of the coding sequence of a gene, between a promoter and the translation start site. This latter application is able to knock down, or effectively prevent, expression of the downstream gene.

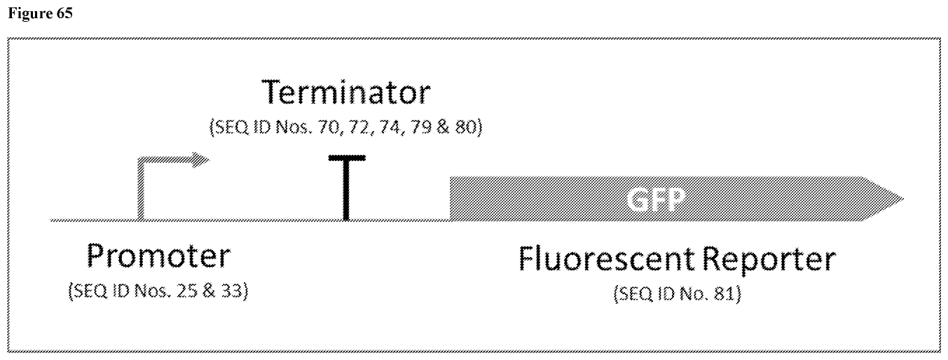

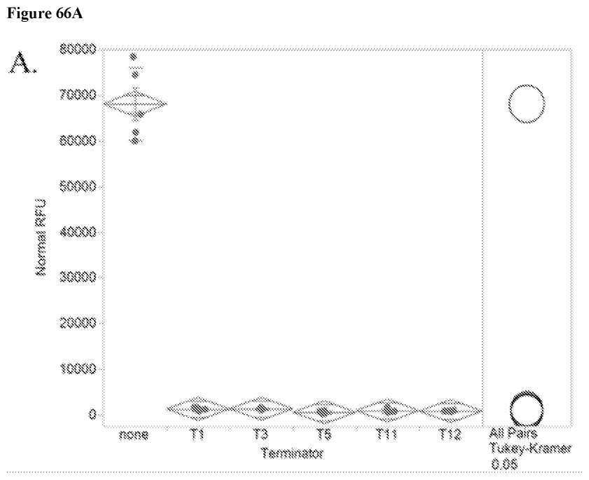

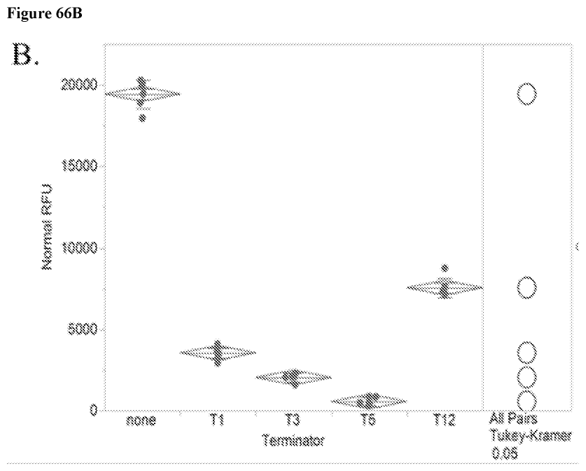

To evaluate the application of this terminator library for knocking down, or eliminating, gene expression, we tested this application by inserting individual terminators (a subset of our terminator library: SEQ ID Nos. 70, 72, 74, 79 & 80) between one of two different promoters (SEQ ID No. 25 and 33) and a fluorescent reporter (SEQ ID No. 81) (FIG. 65). These test casettes were then integrated into Strain A and GFP expression of the resulting strains was used to evaluate the effect of the terminator insertions on attenuation of GFP expression (FIG. 66A-B). FIG. 66A shows expression of strains with T1, T3, T5, T11 and T12 (SEQ ID Nos. 70, 72, 74, 79 & 80) inserted between a strong promoter (SEQ ID No. 25) and GFP. “None” (left column) indicates the no-terminator control strain. FIG. 66B shows expression of strains with T1, T3, T5 and T12 (SEQ ID Nos. 70, 72, 74 & 80) inserted between a moderately strong promoter (SEQ ID No. 33) and GFP. “None” (left column) indicates the no-terminator control strain. Standard deviations are indicated by the horizontal dashes, typically observed above and below the diamonds. Circles on the rights side of the figure indicate significant differences between groups (non-overlapping/intersecting circles indicate groups that are significantly different from each other) based on Tukey-Kramer HSD test of all pairs.

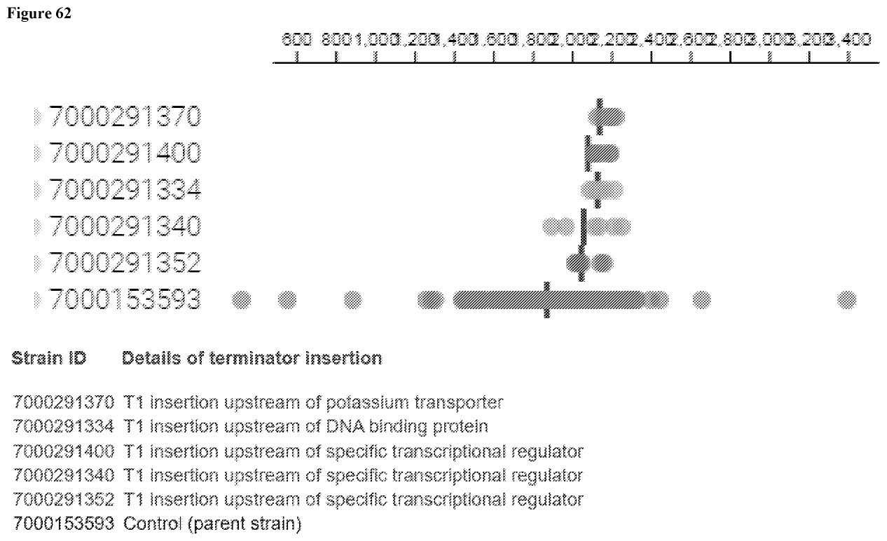

Data demonstrating the utility of this engineering approach is shown in FIG. 62. Terminators were inserted upstream of a number of targeted genes to modify gene expression, and these engineered strains were tested in comparison to parent strain in a plate assay for polyketide productivity. Several “terminator insertion” strains exhibited an improvement compared to the parent strain. In some embodiments, collections of terminator insertions (sequences, terminator-gene combinations, or strains) are referred to as a “terminator insertion microbial library.”

### Example 9: Rapid Consolidation of Genetic Changes and for Generating Genetic Diversity in Saccharopolyspora

This example demonstrates methods for rapid consolidation of genetic changes and for generating genetic diversity in Saccharopolyspora spinose. Engineering of S. spinosa strains is a lengthy process largely due to the slow growth and lack of genetic tools for the organism. This problem is further exacerbated in production strains, which are more likely to have reduced growth rates and reduced robustness. For example, the method for engineering S. spinosa before the present invention is to introduce foreign DNA by conjugation (Matsushima et al, 1994. Gene, 146 39-45). The process is based on single cross-over of the delivered plasmid into the host DNA. The process to introduce foreign DNA and select the strains of interest takes approximately 14-21 days. If the engineering has to be “scar-less”, the elements of plasmids used to deliver the mutations (e.g., plasmid backbone) must be removed after the initial integration, leaving only the “pay-load”. The “pay-load” is the desired mutation, which can be a single nucleotide polymorphism (SNP), a change in the gene promoter, a change in the ribosomal binding site, a change of the gene terminator, a multigene cassette, any genetic element having about 1-10000 bp in size, or a deletion of any size. The removal of the elements of delivery plasmid adds another ˜20 days to the engineering process. In some instances, removal of the elements of delivery plasmids is not immediately required, as is the case for full gene integrations at a neutral site. In those cases the plasmid, and the plasmid-encoded selectable (kanR) and counter-selectable (sacB) markers, are retained in the host chromosome, and the mutation is considered “marked”. The traditional way of combining such mutations is to generate the first mutation into a base strain through integration and counter-selection (˜45 days) thus generating a mutant strain (Mut1 for example) and then proceed to repeat the process with the next mutation using the Mut1 strain as a recipient and going through the 45 day engineering process again thus generating a new strain with two mutations (e.g. Mut2). To add a 3rd mutation it would take a minimum of another 45 days, and so on.

The present disclosure teaches new methods for accelerating the strain improvement programs of host cells, through rapid consolidation of genetic changes. To reduce the engineering time the inventors designed (improved on existing methods) a method for rapid consolidation of rationally engineered mutations. The new methods are based on protoplast fusion of selected strains, such as previously engineered strains, and/or strains that with “good” mutation(s).

### A. General Methods

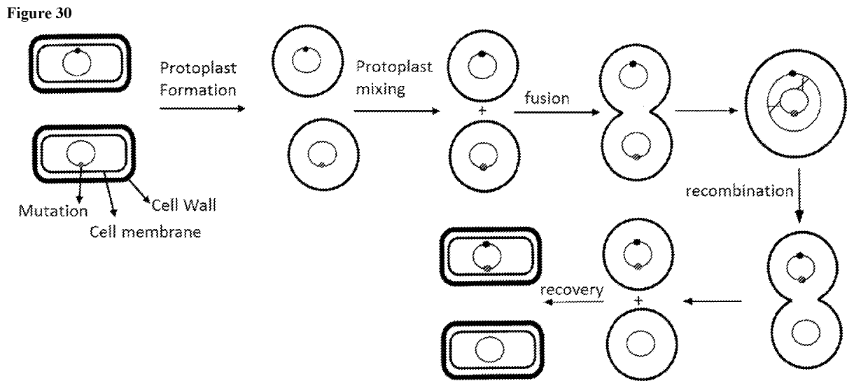

An exemplary procedure for consolidating mutations is demonstrated in FIG. 30. As a starting point, parent strains with genomes containing interested mutations are generated and selected.

In some embodiments, it is desired to have one of those mutations being marked. Once the strains are generated and tested, the best mutations can be rapidly consolidated using the process outlined herein. Briefly, protoplasts are generated form strains of interest and then mixed together at different ratios, with the “marked” strains used at a much lower concentration as compared to unmarked strains. After the fusion, resultant strains are recovered on a media modified for the process, and selection is applied for the “marked” strain thus killing any cells that did not receive the “marked” mutation. HTP strain QC can rapidly determine which of the other mixed mutations are present in the thus selected strains. The expectations is that most strains contain at least one of the other mutations and in some cases more than one.

This process normally takes 7-10 days to generate strains, and a single consolidation reaction can result in several different genotypes depending on the number of mixed strains. For example, a four-way fusion of strains M1, S1, S2, and P1 can result in 4 rare single mutants and 10 different combinations: M1 S1; M1 S2; M1 P1; S1 S2; S1 P1; S2 P1; M1 S1 S2; M1 S2 P1; S1 S2 P1; M1 S1 S2 P1. In addition, S1 S2; S1 P1; S2P1; S1 S2 P1 types will be lost if selection of the marked mutation in M1 is applied.

For example, the methods described herein may contain the following steps:

(1) Choosing parent strains from a pool of engineered strains, then selected strains will be consolidated. In some embodiments, at least one of the stains has a “marked” mutation. Interesting strains used in for parents greatly increase the chances of useful strains being generated in subsequent steps
(2) Preparing protoplasts (e.g., removing the cell wall, etc.) from the strains that are to be consolidated. Cells need to be grown in osmotically stabilized media and buffers, which buffers and media differ from prior art.
(3) Fusing the strains of interest. In some embodiments, to increase the odds of generating useful (novel) combinations of mutants, fewer cells of the stain with “marked” mutation can be used, thus increasing the chances that these “marked” cells would have interacted and fused with cells carrying different mutations. This is the step where cells are fused together and consolidation happens. The exact fraction of strains used during this step would affect the likelihood of obtaining certain combinations.
(4) Recovery of cells. In some embodiments, cells are plated on osmotically stabilized media without the use of agar overlay, which simplifies the procedure and allows for easier automation. The osmo-stabilizers are such that allow for the growth of cells which might contain the counter-selection marker gene (e.g., sacB gene). Protoplasted cells are very sensitive to treatment and are easy to kill. This step ensures that enough cells are recovered. The better this step works, the more material can be used for downstream analysis.
(5) Selection of cells which carry the “marked” mutation. This is accomplished by overlaying appropriate antibiotic onto the growing cells. In case neither of the parent cell carries a “marked” mutation, the strains can be genotyped by other means to identify strains of interest. This step could be optional but it ensures that cells that have most likely undergone cell fusion are enriched. It is possible to “mark” multiple loci and this way one can generate the combinations of interest faster, but then multiple plasmids may have to be removed if one would like to have “scarless” strains.
(6) Genotype growing cells for the presence of mutations coming for the other parent strains. This step looks for the presence of the other mutations that are to be consolidated. The number of colonies to genotype will depend on the complexity of the cross as well as the selection scheme.
(7) (optional) Removing the plasmid form the “marked” mutation. This is optional and is recommended for additional verification or client delivery. In some embodiments, at the end of engineering cycles for a strain, all plasmid remnants need to be removed. When and how often this is carried out is at the discretion of the user. In some embodiments, the presence of the counter-selectable sacB gene makes this step straightforward.

The generated strains can be tested for the desired phenotype of interest. Mutations that are genetically very close on the genome will be harder to consolidate. It will be prudent to know what mutations are selected for consolidation to increase the chances of successful consolidation. In addition, Steps 2, 3, and 4 as described herein are essential for success and if skipped or not executed properly, the outcome of the protocol would be.

In some embodiments, none of the mutations is “marked”. For example, there are no markers genetically linked to the mutations. When in total N (N≥3) different strains where each contains a unique unmarked mutation are consolidated, the methods of the present disclosure provide reduction in circle time through recursive shuffling events and maximized opportunity for recombination between different genomes. In this case, the methods comprise the following steps: (1) Choosing parent strains from a pool of engineered strains, then selected strains will be consolidated; (2) Preparing protoplasts (e.g., removing the cell wall, etc.) from the strains that are to be consolidated; (3) Fusing the strains of interest. In this step, cells are fused together and consolidation happens; (4) Recovering cells; (5) selecting of cells which carry at least one of the interested mutations. This can be done by genotyping or by any other suitable means to identify a mutation of interest; (6) selecting of cells which carry additional one or more interested mutations coming for the other parent strains.

Methods for generating propoplasts include, but are not limited to those described in Kieser et al. (Practical Streptomyces Genetics, John Innes Center, ISBN0708406238).

### B. Results

In one experiment, one marked strain and three unmarked strains each carrying a SNP mutation at different distance from the marked locus. The fused protoplasts will be selected in the presence of antibiotic, which killed all unmarked strains. Then locus of each SNP will be sequenced to verify genetic exchange. Without wishing to be bound by any particular theory, if loci are well separated, exchange may be more frequent.

In another experiment, for producing the fused protoplasts derived from different strains, 1% marked strain and 99% unmarked strain will be mixed and selected. The relative spinosyn production will be tested in the selected strains with consolidated mutations, and compared to the parental strains (both marked and unmarked parental strains). The result will indicate that there is diversity generated: some strains will perform better than both parents, while some strains will perform worse or equally.

In a third example, phenotypic diversity generated by shuffling will be observed and shown. Only cells carrying the marker from the “marked” parent will grow on this media. The observed differences in colony morphology (bald-opaque color, and sporulating (white) cells) and colony sizes (large and small) is indicative of shuffling events. The cells contain the a counter-selection marker, such as sacB marker will be recovered on the R2YE Sorb/Man media.

### Example 10: Reporter Proteins and Related Assays for Use in Saccharopolyspora spinosa

S. spinosa is a largely intractable host with very few molecular biology tools required to support the development of engineering tools and engineering efforts for this organism. Reporter proteins represent critical tools that were lacking for this organism.

A major goal of inventors' genetic engineering efforts, and metabolic engineering more broadly, is to alter host metabolism, optimize biosynthetic pathways, and introduce or duplicate pathway genes in order to improve the yield of a desired product. Success relies on the ability to perturb and balance expression of genes both within (on-pathway) and outside (off-pathway) of the biosynthetic gene cluster or over-express non-native genes or copies of genes that are introduced. These efforts require the development and characterization of libraries genetic (DNA) elements (e.g., promoters, ribosomal binding sites, transcription terminators) that can be employed in engineering designs. Reporter proteins and assays to evaluate their expression are essential for characterization of these libraries.

In this example, the present disclosure provides the demonstration and quantitative evaluation of three reporter genes in Saccharopolyspora spinosa. The three reporter genes described here include two fluorescent reporter proteins (Dasher GFP and Paprika RFP; ATUM, https://www.atum.bio/products/protein-paintbox?exp=2) and the enzyme beta-glucuronidase (gusA) (Jefferson et al. (1986). “Beta-Glucuronidase from Escherichia coli as a gene-fusion marker”. Proceedings of the National Academy of Sciences of the United States of America. 83 (22): 8447-51). The present invention represents the first time that these markers have been successfully employed as molecular tools in S. spinosa. The present disclosure also describes the optimization and application of a colorimetric assay that enables quantitative evaluation of GusA expression in S. spinosa.

The nucleotide sequences encoding DasherGFP (ATUM) and PaprikaRFP (ATUM) were codon-optimized for E. coli. (SEQ ID No. 81 and SEQ ID No. 82). The nucleotide sequence encoding beta-glucuronidase (gusA) was codon optimized for S. spinosa (SEQ ID No. 83).

To test the reporter genes, the ermE* promoter (SEQ ID No. 149) was cloned in front of the reporter coding sequences and the resulting constructs were integrated into a known neutral site in the S. spinosa genome. Resulting strains were grown for 48 hours in liquid culture (growth media). Aliquots of cultures were washed with PBS then either (1) fluorescence measurements were made on aliquots of replicate cultures in 96-well plates using a Tecan Infinite M1000 Pro (Life Sciences) plate reader; (2) absorbance (OD₄₀₅) of cell-free extracts after incubation at 37° C. in the presence of 4-Nitrophenyl β-D-glucuronide, following a modified OpenWetWare protocol for Lactobacillus spp. (http://www.openwetware.org/wiki/Beta-glucuronidase_protocols).

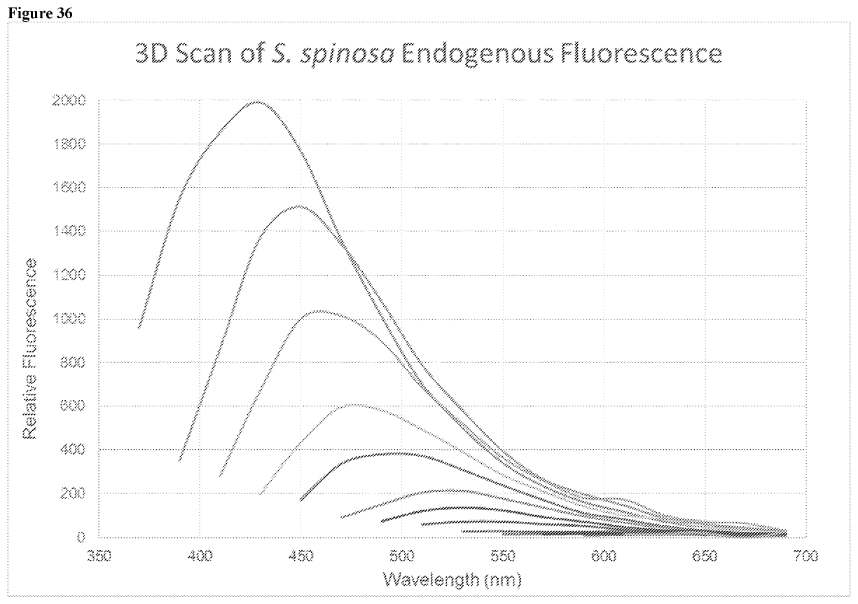

Fluorescence of the reporters DasherGFP and PaprikaRFP were measured in S. spinosa strains engineered to contain such reporters. The results show that both reporters work in S. spinosa and that they have distinct fluorescence signatures (see FIG. 31A-D). This is unexpected because even though the nucleotide sequences encoding the reporters DasherGFP and PaprikaRFP were optimized for E. coli, they resulted in expression of the proteins in S. spinosa. This may not have been the case had we selected different reporter genes. Also, the fluorescent proteins selected had spectra that did not overlap the spectrum of endogenous fluorescence observed in S. spinosa (FIG. 36).

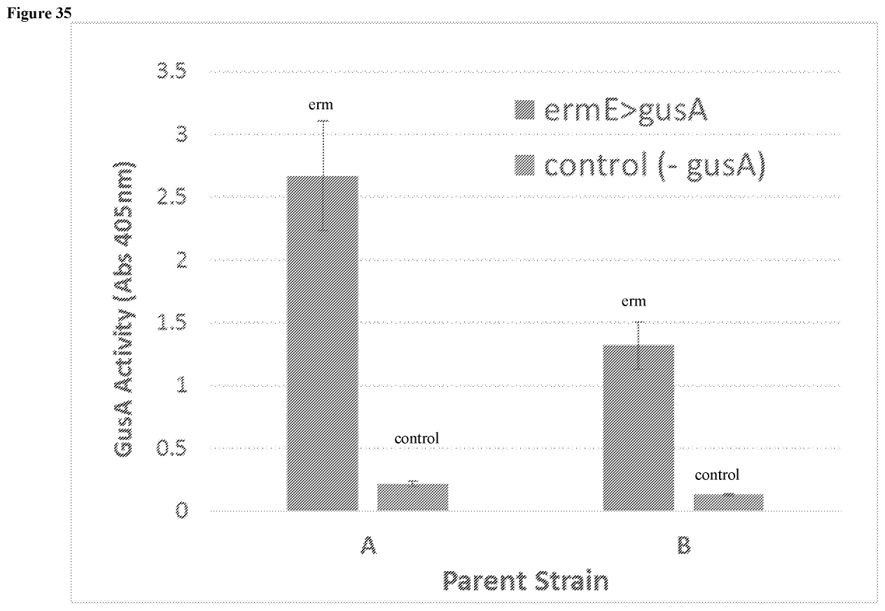

GusA activity of the optimized beta-glucuronidase (gusA) in S. spinosa were measured using a colorimetric 4-Nitrophenyl β-D-glucuronide assay developed for use in Lactobacillus spp. (Jefferson et al. (1986). “Beta-Glucuronidase from Escherichia coli as a gene-fusion marker”. Proceedings of the National Academy of Sciences of the United States of America. 83 (22): 8447-51.) The results indicate that the 4-Nitrophenyl β-D-glucuronide assay (including the cell lysis and enzymatic reaction) developed for use in Lactobacillus spp. also works in S. spinosa (FIG. 35).

The GusA assay protocol is briefly described as below:

1. Grow culture until OD600 is between 0.6 and 1.0

2. Prepare 10 mL of GUS Buffer (measures 10 samples) by adding:
5 mL of sodium phosphate buffer (pH=7)
3 mL H2O
1 mL of potassium chloride solution
1 mL of magnesium sulfate solution
354 β-mercaptoethanol
20 mg Lysozyme

3. Pellet 1.5 ml of culture by centrifugation for 1 minute.

4. Resuspend in 1 ml 100 mM sodium phosphate buffer, which contains:
0.1M potassium chloride solution
10 mM magnesium sulfate solution
1M Na2CO3
4-Nitrophenyl β-D-glucuronide (4-NPG) stock solution (10 mg/ml in 50 mM sodium phosphate buffer (pH=7)) only make 1 mL of this!!!
β-mercaptoethanol
10% Triton X-100 (in water)

5. Pellet again by centrifugation.

6. Resuspend in 750 μL GUS buffer.

7. Vortex briefly to mix.

8. Incubate for 30 min in 37° C. water bath.

9. Add 8 ul of 10% Triton-X.

10. Vortex briefly and incubate on ice for 5 mins.

11. Add 80 ul of 4-NPG solution and start the timer.

12. Incubate in 37° C. water bath.

13. When the color is clearly yellow (between 10 and 30 mins), stop reaction by adding 300 μM Na2CO3

14. Record the time.

15. Centrifuge the reaction for 1 minute at full speed.

16. Measure the OD405 of the supernatant.

This invention enables the quantitative evaluation of such libraries but also has other potential applications (e.g., use in the development of biosensors and screening of colonies, and a marker and target for demonstration of gene-editing technologies). The three reporters described here are the first reporter genes and quantitative assays developed for use in S. spinosa. Additionally, they have the benefit of being common reporters in other biological systems, and, as such, it is possible to use established methods and instruments already optimized for their detection.

### Example 11: HTP Genomic Engineering—Integrase Based System for Targeted and Efficient Genomic Integration in Saccharopolyspora spinosa

Integration of exogenous DNA is an effective method for improving strain performance, however this is highly inefficient in S. spinosa, particularly for large pieces of DNA (>10 kb). The ability to duplicate and refactor biosynthetic pathways in hosts like S. spinosa is critical for metabolic engineering efforts, however the sizes of these pathways make these efforts prohibitive.

The present Example describes an integrase-based system for integration of genetic elements into the genome of S. spinosa. Integrases direct targeted integration of DNA payloads through recognition and attachment at conserved sites (att sites; conserved nucleotide sequences typically located within tRNA genes in the host chromosome). We anticipate that the integrase-based system described by this invention would allow for the delivery of genetic payloads tens of kilobases in size, thereby enabling efficient introduction of exogenous DNA from heterologous organisms or duplication of native genes from S. spinosa. We anticipate being able to show that one or multiple of the following selected integrases enable efficient introduction of DNA to specific sites in the genome:

TABLE 10
Integrases for integration of genetic
elements into the genome of S. spinosa.

| Integrase | Origin | Sequences |
|---|---|---|
| pCM32 | S. endophytica | PCM32integrase + attP (SEQ ID No. 84) |
| [1] | | atgccgcgtaagaaccgcgatgaaggcacccgggcgcccaacggcgcgagcagca |
| | | tctacaagggcaaagacggctactggcacggccgcgtctggatgggcaccaagga |
| | | cgacggcagtgaggaccgtcgccacaggtcagcgaagagcgaaacagagctcctc |
| | | aataaggttcgcaagctcgaacgggagcgggacagcggcaaggtgcagaagcctg |
| | | gccgcgcctggaccgtcgagaaatggcttacgcactgggtggagaacatcgccgc |
| | | tcccaccgtgcggccgaccacgatggtcggctaccgcgcctcggtgtataagcat |
| | | ctgatccccggcgtgggcaagcaccggatcgacaggttgcagccggaacacctcg |
| | | aaaagctctacgccaagatgcagcgcgatggactcaaggccgcgacagcgcacct |
| | | cgcgcaccggacggtgcgggtcgcgctgaacgaggccaagaagcgacgtcacatc |
| | | accgagaacccggccaatatcgcgaagccgcccagggtggacgaggaggagattg |
| | | tcccgttcacggtggatgaagcccgccggatcctcgcagcagctgcggagacgcg |
| | | gaacggcgctcgctttgtcatcgcgctgacccttggcctgcgcaggggtgaagca |
| | | ctcgggttgaagtggtcggatctctcgatcacctggaagcacggatgccggaagg |
| | | ggagcgcgtgccgggtgggtcgccgagccgagcagtgcggcgagcgtcgcggcag |
| | | cggcacgctcgtcatccggcgcgcgattcagcagcaggtttggcagcacggttgc |
| | | tcagaggacaagccgtgcgaccaccgctacggcgctcactgcccgcgccggcata |
| | | gcggcggtgtggtcgtgaccgatgtgaagtccagggcgggtcggcgaaccgtggg |
| | | ccttccgcacccggtggtggaagcgctcgaagagcaccgcgcccgccagcggaca |
| | | gagcgggagaaggcgcgcaacgagtgggacgacgccgattgggtcttcacgaaca |
| | | ggtggggtcgcccggttcatccgaccgttgactacgacgcctggaaggcactgct |
| | | cagggcagcgaacgtgcgcaacgcgcggttgcacgacgcacgccacaccgcggcg |
| | | acgatgttgctggtgttgaaggttccgctgcctgcggtcatggaaatcatgggct |
| | | ggtcggaagcctctatggccaagcgctacatgcacgtgccgcacgagctcgtgac |
| | | cgcgatcgcggaccaggtgggtgacctggtgtggcccgtcccagagaccgaggag |
| | | gaggcgccaccgcctgaggaggagtgggcgctggacgccaaccaggtggcggcga |
| | | tccggaagctggccggagctctcccgccgcagttgcgggagcagttcgaggcgct |
| | | gctgcccggcgacgacgaggacgacggcccgacttcgggagtggtcatccctgcg |
| | | taaccagtgcggccagaacccggcctaacggggcctactgagacgaaaactgaga |
| | | ctggacatgcgagaggcccggaagcgagatcgcttccgggcctctgacctgcgga |
| | | ggatacgggattcgaacccgtgagggctattaacccaacacgatttccaattccg |
| | | atggcgcgagtgccagggggtagctgaacgtgccttttgcctggtcagtggcact |
| | | acggcaacatcaggtgtggcttgatccgtgcgcgt |
| | | >pCM32integrase_protein (SEQ ID No. 85) |
| | | MPRKNRDEGTRAPNGASSIYKGKDGYWHGRVWMGTKDDGSEDRRHRSAKSETELL |
| | | NKVRKLERERDSGKVQKPGRAWTVEKWLTHWVENIAAPTVRPTTMVGYRASVYKH |
| | | LIPGVGKHRIDRLQPEHLEKLYAKMQRDGLKAATAHLAHRTVRVALNEAKKRRHI |
| | | TENPANIAKPPRVDEEEIVPFTVDEARRILAAAAETRNGARFVIALTLGLRRGEA |
| | | LGLKWSDLSITWKHGCRKGSACRVGRRAEQCGERRGSGTLVIRRAIQQQVWQHGC |
| | | SEDKPCDHRYGAHCPRRHSGGVVVTDVKSRAGRRTVGLPHPVVEALEEHRARQRT |
| | | EREKARNEWDDADWVETNRWGRPVHPTVDYDAWKALLRAANVRNARLHDARHTAA |
| | | TMLLVLKVPLPAVMEIMGWSEASMAKRYMHVPHELVTAIADQVGDLVWPVPETEE |
| | | EAPPPEEEWALDANQVAAIRKLAGALPPQLREQFEALLPGDDEDDGPTSGVVIPA |
| | | * |
| | | >attP site in pCM32 (SEQ ID No. 167) |
| | | Gcgagaggcccggaagcgagatcgcttccgggcctctgacctgcggaggatacgg |
| | | gattcgaacccgtgagggctattaacccaacacgatttccaattccgatggcgcg |
| | | agtgccagggggtagctgaacgtgccttttgcctggtcag |
| pSE101 | S. erythraea | pSE101integrase + attP (SEQ ID No. 86) |
| [2] | | atgccccgcaaacgccgcccagaaggcacccgagcccccaacggcgccagcagca |
| | | tctactacagcgagacggacggctactggcacgggcgcgtcacgatgggcgtccg |
| | | cgacgacggcaagcccgaccgtcgccacgtccaagccaagaccgagaccgaggtc |
| | | atcgataaggtccgcaagctcgaacgtgaccgggatagcggcaacgcgcggaagc |
| | | ctggtcgcgcgtggacagtcgagaagtggctgactcactgggtcgagaacatcgc |
| | | ggtgcactccgttcggtacaagacgcttcagggctaccgaacggcggtctacaag |
| | | cacctgatccccggtatcggcgcgcaccggatggaccgcatcgagccggagcact |
| | | tcgagcggttctacgccaggatgcaggccgccggcgccagtgcagggaccgcaca |
| | | tcaggtgcaccggactgccaaaacggcattcaacgaatacttccggcggcagcgc |
| | | atcaccgggaaccccatcgccttcgtgaaagcgccgcgcgtcgaggaaaaggaag |
| | | tggagccgttcacgccgcaggaagccaagagcatcatcacggccgcgctcaagcg |
| | | gcgcaacggcgtgcgatacgtcgtcgccttggctctcggttgtcgccaaggcgaa |
| | | gccctggggttcaagtgggaccgcctcgaccgcgggaaccggctttaccgcgtac |
| | | ggcaggcattgcagcggcaggcttggcaacacggatgcgacgacccgcacgcctg |
| | | cggagcacgacttcatcgggtggcgtgcccggacaactgcacccagcatcgcaac |
| | | cgcaagagctgcattcgcgacgagaagggccaccaccgtccgtgcccgccgaact |
| | | gcaccaggcacgcgagcagttgcccgcagcggcacggtggtgggctcgtcgaggt |
| | | cgacgtgaagtcgaaggctggtcgccggagcttcgttctgccagatgaggtcttc |
| | | gatctgctgatgcgccacgagcaggcgcagcagcgggagcgcaagcacgccggta |
| | | gcgagtggcaggaggggggctgggtcttcacccagcccaacggccggccgatcga |
| | | tccgcggcgcgactggggtgagtggaaggacatcttgggggaggcaggtgttcgg |
| | | gatgctcggctgcacgacgcgcgccacactgcggcgacggtcctcatgctgctcc |
| | | gcgttccagaccgggccgtccaggatcacatgggctggtcctcgatccggatgaa |
| | | ggagcgctacatgcacgtcaccgaggaactgcgacgagagatcgccgatcagctc |
| | | aacgggtacttctgggacgtcaactgagacggaaagtgagacgaaaagcgcctgg |
| | | tcagggacctgtcgacggcgtttccgctggtagtttcggagccgctgaggggact |
| | | cgaacccctgaccgtccgcttacaaggcgggcgctctaccaactgagctacagcg |
| | | gcgtgcgctacgtcgcgcgcgaacatcgtaagcgtccacc |
| | | >pSE101integrase_protein (SEQ ID No. 87) |
| | | MPRKRRPEGTRAPNGASSIYYSETDGYWHGRVTMGVRDDGKPDRRHVQAKTETEV |
| | | IDKVRKLERDRDSGNARKPGRAWTVEKWLTHWVENIAVHSVRYKTLQGYRTAVYK |
| | | HLIPGIGAHRMDRIEPEHFERFYARMQAAGASAGTAHQVHRTAKTAFNEYFRRQR |
| | | ITGNPIAFVKAPRVEEKEVEPFTPQEAKSIITAALKRRNGVRYVVALALGCRQGE |
| | | ALGFKWDRLDRGNRLYRVRQALQRQAWQHGCDDPHACGARLHRVACPDNCTQHRN |
| | | RKSCIRDEKGHHRPCPPNCTRHASSCPQRHGGGLVEVDVKSKAGRRSEVLPDEVF |
| | | DLLMRHEQAQQRERKHAGSEWQEGGWVFTQPNGRPIDPRRDWGEWKDILGEAGVR |
| | | DARLHDARHTAATVLMLLRVPDRAVQDHMGWSSIRMKERYMHVTEELRREIADQL |
| | | NGYFWDVN* |
| | | >attP site in pSE101 (SEQ ID No. 168) |
| | | Tcggagccgctgaggggactcgaacccctgaccgtccgcttacaa |
| pSE211 | S. erythraea | >pSE211integrase + attP (SEQ ID No. 88) |
| [3] and | | acgtcacccaactcgccgccacgctcgcctcgctcgcggccctgctcgccgaaca |
| [4] | | gcagcccgccccggaacccgagcccgaaccggccgcccgcaggctgcccaaccgc |
| | | gtgctgctcacggtcgaggaagcggccaagcaactggggctcggcaggaccaaga |
| | | cctacgcgctggtggcgtctggcgagatcgaatctgtccggatcggtcggctcag |
| | | gcgcatcccgcgcaccgccatcgacgactacgccgcccgactcatcgcccagcag |
| | | agcgccgcctgaagggaaccactatggaacaaaagcgcacccgaaaccccaacgg |
| | | tcgatcgacgatctacctcgggaacgacggctactggcacggccgcgtcaccatg |
| | | ggcatcggcgacgacggcaagcctgaccggcgccacgtcaagcgcaaggacaagg |
| | | acgaagttgtcgaggaggtcggcaagctcgaacgggagcgggactccggcaacgt |
| | | ccgcaagaagggccagccgtggacagtcgagcggtggctgacgcactgggtggag |
| | | agcatcgcgccgctgacctgccggtacaagaccatgcggggctaccagacggccg |
| | | tgtacaagcacctcatccccggtttgggcgcgcacaggctcgatcggatccagaa |
| | | ccatccggagtacttcgagaagttctacctgcgaatgatcgagtcgggactgaag |
| | | ccggcgacggctcaccaggtacaccgcacggcgcgaacggctttcggcgaggcgt |
| | | acaagcggggacgcatccagaggaacccggtttcgatcgcaaaggcacctcgggt |
| | | ggaagaggaggaggtcgaaccgcttgaggtcgaggacatgcagctggtcatcaag |
| | | gccgccctggaacgccgaaacggcgtccgctacgtcatcgcactggctctcggaa |
| | | ctcggcagggcgaatcgctcgcgctgaagtggccgcggctgaaccggcagaagcg |
| | | cacgctgcggatcaccaaggcactccaacgtcagacgtggaagcacgggtgctct |
| | | gacccgcatcggtgcggcgcgacctaccacaagaccgagccgtgcaaggcggcct |
| | | gcaagcggcacacgcgagcttgtccgccgccatgcccgccagcttgcaccgaaca |
| | | cgcccggtggtgcccgcagcgaaccggtggcgggctggtcgaggtcgacgtcaag |
| | | tcgagggctggacgacggaccgtgacgctgcccgaccaactgttcgacttgatcc |
| | | tcaagcacgaaaagcttcagggggccgaacgggagctcgcgggcacggagtggca |
| | | cgacggcgagtggatgttcacccagcccaacggcaagccgatcgatccacgtcag |
| | | gacctcgacgagtggaaagcaatccttgttgaagccggagtccgcgaggcgcggc |
| | | tacatgacgcacggcacaccgccgcgactgtgctgttggtcctcggagtgcccga |
| | | ccgggtcgtgatggagctgatgggctggtcgtccgtcaccatgaagcagcggtac |
| | | atgcacgtcatcgactccgtccggaacgacgtagcggaccgcctgaacacctact |
| | | tctggggcaccaactgagacccagactgagacccaaaacgcccccgtcgagatcg |
| | | acgggggcgttttggcagctcttggtggtggccaggggcggggtcgaaccgccga |
| | | ccttccgcttttcaggcggacgctcgtaccaactgagctacctggccgttcgcgc |
| | | ccggctcaaagccgaaccgctgtggcgacccagacgggactcgaacccgcgacct |
| | | ccgccgtgacagggcggcgcgctaaccaactgcgccactgggccatgttctgttg |
| | | ttgcgtacccccaacgggattcgaacccgcgctaccgccttgaaagggcggcgtc |
| | | ctaggccgctagacgatgggggcttggccgattcggaaccgacccggcctcgcct |
| | | ccaaccggctttccctttcggggcgccccgttgggagcagtgaaagcttacgaca |
| | | caccccccagcgccccacaacgggggggtccccaaacctcacgagcccccgcgcg |
| | | gcccacgcccgccggtcacgtcggtcgccaccatatgccatctgaccagcctttt |
| | | ccatcgcctatcctcagtcggcccact |
| | | >pSE211integrase_pro (SEQ ID No. 89) |
| | | MEQKRTRNPNGRSTIYLGNDGYWHGRVTMGIGDDGKPDRRHVKRKDKDEVVEEVG |
| | | KLERERDSGNVRKKGQPWTVERWLTHWVESIAPLTCRYKTMRGYQTAVYKHLIPG |
| | | LGAHRLDRIQNHPEYFEKFYLRMIESGLKPATAHQVHRTARTAFGEAYKRGRIQR |
| | | NPVSIAKAPRVEEEEVEPLEVEDMQLVIKAALERRNGVRYVIALALGTRQGESLA |
| | | LKWPRLNRQKRTLRITKALQRQTWKHGCSDPHRCGATYHKTEPCKAACKRHTRAC |
| | | PPPCPPACTEHARWCPQRTGGGLVEVDVKSRAGRRTVTLPDQLFDLILKHEKLQG |
| | | AERELAGTEWHDGEWMFTQPNGKPIDPRQDLDEWKAILVEAGVREARLHDARHTA |
| | | ATVLLVLGVPDRVVMELMGWSSVTMKQRYMHVIDSVRNDVADRLNTYFWGTN* |
| | | >attP site in pSE211 (SEQ ID No. 169) |
| | | ggcagctcttggtggtggccaggggcggggtcgaaccgccgaccttccgctt |
| pSE101 | S. spinosa | >SS101_homolog(3g00449)_CDS + attP (SEQ ID No. 90) |
| homolog | | atgccacgcaaacgccgcccggaaggcacccgggcacccaacggagccagcagca |
| | | tctacctcggcaaggacggctactggcacggccgcgtcaccgtcggagttcgcga |
| | | cgacggtaagcccgaccgccctcacgtccaggccaagaccgaggccgaagtcatc |
| | | gacaaggtgcgcaagctcgaacgcgatcgcgatgcggggaaggtgcgaaagcctg |
| | | gccgggcctggaccgtcgagaagtggcttacgcactgggtcgagaacatcgccgc |
| | | gccatccgtccgttacaagacccttcagggctaccgcacggcggtgtacaagcac |
| | | ttgatccccggcatcggcgcgcaccggatcgaccgaattgaaccggagcacttcg |
| | | agaagctctacgcgaagatgcaggaatccggcgcgaaagcgggaaccgcgcacca |
| | | ggtgcaccgcaccgctcgggccgcctttaacgaagccttccggcgtcggcacctc |
| | | accgaaagcccggtgcggttcgtgaaagcgccgaaggtcgaagaagaggaagtcg |
| | | agcccttcacgccgaaggaagcccagcagatcattacggccgcgctcaatcgtcg |
| | | aaacggcgtgcgattcgtgatcgctctcgcactgggctgccgccagggtgaagcg |
| | | ctgggcttcaagtgggaacggctcgaccgggaaaacaggctctaccacgttcgga |
| | | gggcgcttcagcgtcaagcctggcaacacggctgtgaagatccgcacaactgcgg |
| | | tgcgaggttccaccgggttgcttgcgccgagaactgcaagcggcaccgcaatcgg |
| | | aagaactgcattcgcaacgagaagggacacgctcgaccgtgcccgccgaactgcg |
| | | accgacacgccagcagctgcccgaaacggcacggcggaggcctgcgcgaggtgga |
| | | tgtgaagtcgaaggctggccgccggcggttcgttcttcctgacgagatcttcgac |
| | | ctgctcatgcggcatgaggaagtccagcggcacgaacgggttcacgccggtaccg |
| | | agtggcaggagggcggctggatcttcacgcagcccaacggcaggccgatcgatcc |
| | | gcgccgcgattggggcgagtggaaggagatcctcgcggaggccggtgttcgggat |
| | | gcccggctgcacgacgcgcggcacaccgcagcgacggtgctcatgctgctccgtg |
| | | ttccggaccgggccgttcaggaccacatgggatggtcgtcgatccggatgaaaga |
| | | gcggtacatgcacgtcaccgaggaactgcgccgcgagatcgccgatcagctgaat |
| | | gggtatttctggaaccccaactgagaccgaaagtgagacggatcgcgcctggtca |
| | | ccgggtgggcaggcgcgtttccgctggtacggtcggagccgctgaggggactcga |
| | | acccctgaccgtccgcttacaaggcgggcgctctaccaactgagctacagcggca |
| | | tgcacttcgtcgtgcggggacatcgtaagcggcgat |
| | | >SS101_homolog(3g00449)_protein (SEQ ID No. 91) |
| | | MPRKRRPEGTRAPNGASSIYLGKDGYWHGRVTVGVRDDGKPDRPHVQAKTEAEVI |
| | | DKVRKLERDRDAGKVRKPGRAWTVEKWLTHWVENIAAPSVRYKTLQGYRTAVYKH |
| | | LIPGIGAHRIDRIEPEHFEKLYAKMQESGAKAGTAHQVHRTARAAFNEAFRRRHL |
| | | TESPVRFVKAPKVEEEEVEPFTPKEAQQIITAALNRRNGVREVIALALGCRQGEA |
| | | LGEKWERLDRENRLYHVRRALQRQAWQHGCEDPHNCGAREHRVACAENCKRHRNR |
| | | KNCIRNEKGHARPCPPNCDRHASSCPKRHGGGLREVDVKSKAGRRREVLPDEIFD |
| | | LLMRHEEVQRHERVHAGTEWQEGGWIFTQPNGRPIDPRRDWGEWKEILAEAGVRD |
| | | ARLHDARHTAATVLMLLRVPDRAVQDHMGWSSIRMKERYMHVTEELRREIADQLN |
| | | GYFWNPN* |
| | | >attP site in pSE101 homolog (SEQ ID No. 170) |
| | | tcggagccgctgaggggactcgaacccctgaccgtccgcttacaaggc |
| pSE211 | S. spinosa | >SS211_homolog(3g00347)_CDS + attP (SEQ ID No. 92) |
| homolog | | atgccacgcaagcgccgcccggaaggcacccgggcacccaacggagccagcagca |
| | | tctacctcggaaacgacggctactggcacggccgcgtcacgatgggaacccgtga |
| | | cgacggccgccccgaccgacggcatgtccagggcaagaccgaggccgaagtcata |
| | | gacaaagtgcgcaagctcgaacgcgaccgcgacgccggacggatgcgcaagcctg |
| | | gccgggcctggaccgtcgagaagtggctgatgcactggctggagcacattgcgaa |
| | | gccatcggtccggccgaaaaccgtcgcccggtatcggacttccgtcgagcaatac |
| | | ctgattcctggtctcggtgcgcaccgcatcgaccgcttgcagccggagaacattg |
| | | agaagctgtacgcaaaattgctcgctcgcgggttggcgccgtccactgtgcacca |
| | | tgttcaccggactctgcgcgtcgctttcaacgaggcgttcaagcgggaacacatc |
| | | acgaaaaacccggtcctcgttgcgaaagcgccgaagctggtcgaaccggagatcg |
| | | agccgttcaccgtggccgaagcacaacgaattctcgatgttgcacggacacggcg |
| | | gaatggtgctcggttcgcactcgcgctcgcgctgggaatgcgccagggcgaagct |
| | | ctcggactcaagtggtccgacctgcgaatcacctggcaccacgggtgcgcatccg |
| | | gactcaccgaagaacagcaggcggccatcgaaatgctcgcgaaggtcgatccgca |
| | | gcgatggaagcggcctgacgattccgggtgcggattcaaggacgtggaggactgc |
| | | ccgcaggctcacccggccgcgacactgaacattcggcgcgcattgcagcgccaca |
| | | cctggcaacacgggtgcggtgacaaaccgacgtgcggcaagaaacggggcgcgga |
| | | ctgcccgcagcgtcatggcggcggcttggccatcgtcccggtgaagtcgagggcg |
| | | gggacgcgctcgatcagcgtgcctgagccgctgattcatgcgttgctcgatcacg |
| | | acgaggcgcaggatgaggaacggcacttggcccggaacctgtggcacgacgatgg |
| | | atggatgttcgctcagcccaacgggaaggcgacggacccgagggccgactatggc |
| | | gaatggcgcgagctgctggacgccgcgaaggttcggccggcgcggctgcacgacg |
| | | cgcggcacaccgccgcgacgatgttgctggttctcaaggtcgcaccacgggcaat |
| | | catggacgtgatgggctggtcggaggcgtcgatgctgacccgctacgtccacgtg |
| | | ccggacgagatcaagcagggcatcgcgggccaggtcggcggactgctgtggaagg |
| | | actggcagcagcccgacgacggcccagacgacgaggacggcggcaccgccgggca |
| | | ccctgtcccggcctgacgtgcccactgccagaggaggcgtttgagccggaaactg |
| | | agccggaacgacaccaggcgctttccgtgtccacggaaagcgcctggtgagagcg |
| | | gagccgcctaagggaatcgaacccttgacctacgcattacgagtgcgtcgctcta |
| | | gccgactgagctaaggcggcgttgcacggccaagtgtagcgggccggacctcgcc |
| | | gtcgttcatggccccgact |
| | | >SS211_homolog (3g00347)_protein (SEQ ID No. 93) |
| | | MPRKRRPEGTRAPNGASSIYLGNDGYWHGRVTMGTRDDGRPDRRHVQGKTEAEVI |
| | | DKVRKLERDRDAGRMRKPGRAWTVEKWLMHWLEHIAKPSVRPKTVARYRTSVEQY |
| | | LIPGLGAHRIDRLQPENIEKLYAKLLARGLAPSTVHHVHRTLRVAFNEAFKREHI |
| | | TKNPVLVAKAPKLVEPETEPFTVAEAQRILDVARTRRNGARFALALALGMRQGEA |
| | | LGLKWSDLRITWHHGCASGLTEEQQAATEMLAKVDPQRWKRPDDSGCGEKDVEDC |
| | | PQAHPAATLNIRRALQRHTWQHGCGDKPTCGKKRGADCPQRHGGGLAIVPVKSRA |
| | | GTRSISVPEPLIHALLDHDEAQDEERHLARNLWHDDGWMFAQPNGKATDPRADYG |
| | | EWRELLDAAKVRPARLHDARHTAATMLLVLKVAPRAIMDVMGWSEASMLTRYVHV |
| | | PDEIKQGIAGQVGGLLWKDWQQPDDGPDDEDGGTAGHPVPA* |
| | | >attP site in pSE211 homolog (SEQ ID No. 171) |
| | | ggagccgcctaagggaatcgaacccttgacctacgcattacgagtgcgtcgctct |
| | | agccgactgagctaaggcggc |

[1] Chen J, Xia H, Dang F, Xu Q, Li W, Qin Z. Characterization of the chromosomal integration of Saccharopolyspora plasmid pCM32 and its application to improve production of spinosyn in Saccharopolyspora spinosa. Applied Microbiology and Biotechnology. PMID 26260388 DOI: 10.1007/s00253-015-6871-z
[2] Brown DP, Chiang SJ, Tuan JS, Katz L (1988a) Site-specific integration in Saccharopolyspora erythraea and multisite integration in Streptomyces lividans of actinomycete plasmid pSE101. J Bacteriol 170:2287-2295
[3] Brown, D.P., Idler, K.B. and Katz, L. (1990) Characterization of the genetic elements required for site-specific integration of plasmid pSE211 in Saccharopolyspora erythraea. J. Bacteriol., 172, 1877-1888.
[4] Te Poele E. M., Bolhuis H., Dijkhuizen L. (2008) Actinomycete integrative and conjugative elements. Antonie Van Leeuwenhoek 94, 127-143.

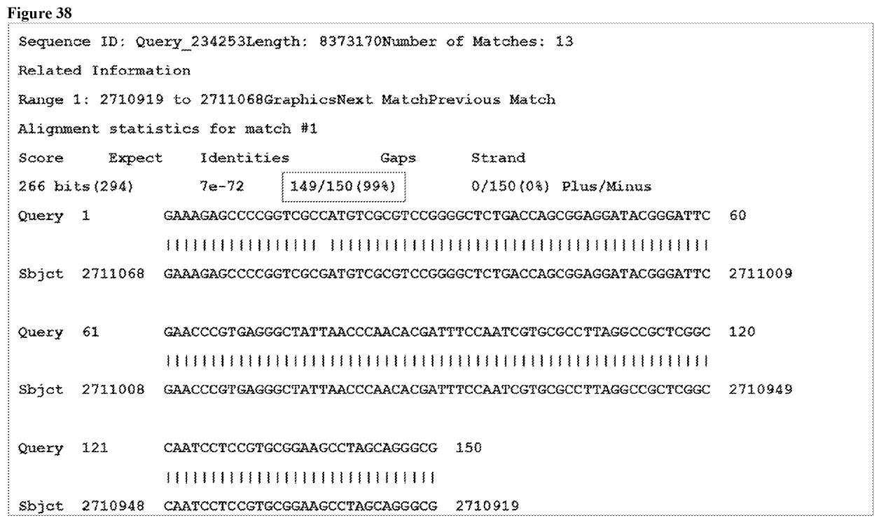

The pCM32 integrase has been shown to work in S. spinosa (Chen et al., “Characterization of the chromosomal integration of Saccharopolyspora plasmid pCM32 and its application to improve production of spinosyn in Saccharopolyspora spinosa. Applied Microbiology and Biotechnology.” PMID 26260388 DOI: 10.1007/s00253-015-6871-z). This is not surprising, as an attachment site with 99% identity to the pCM32 attachment site is found in the S. spinosa genome (FIG. 38). The authors of Chen et al. achieved targeted integration of two genes that resulted in a strain with improved spinosyn titer (see Patent Application CN 105087507A, incorporated by reference in its entirety).

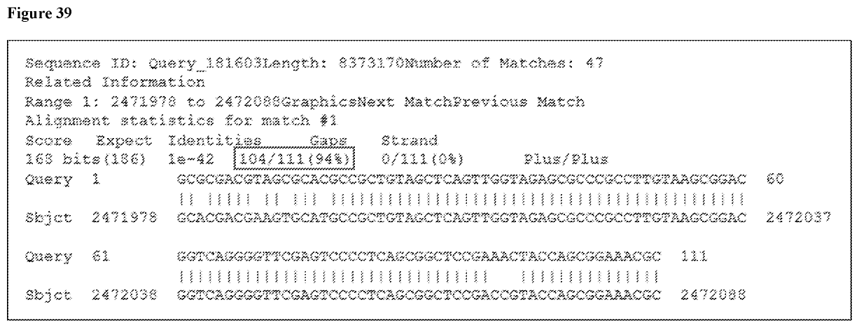

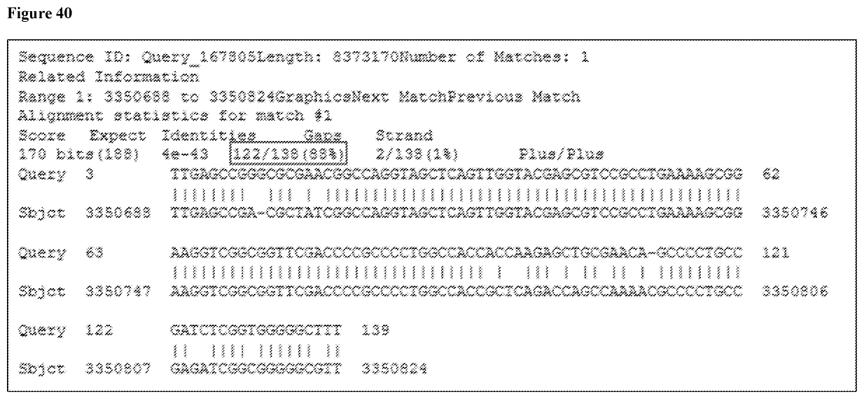

The pSE101 and pSE211 integrases and their attachment sites have been described. The cores of both the pSE101 and pSE211 attachment sites are found in S. spinosa (FIG. 39 and FIG. 40, respectively). These integrase systems were tested and did not work. Inventors will test modified systems and other integrase system.

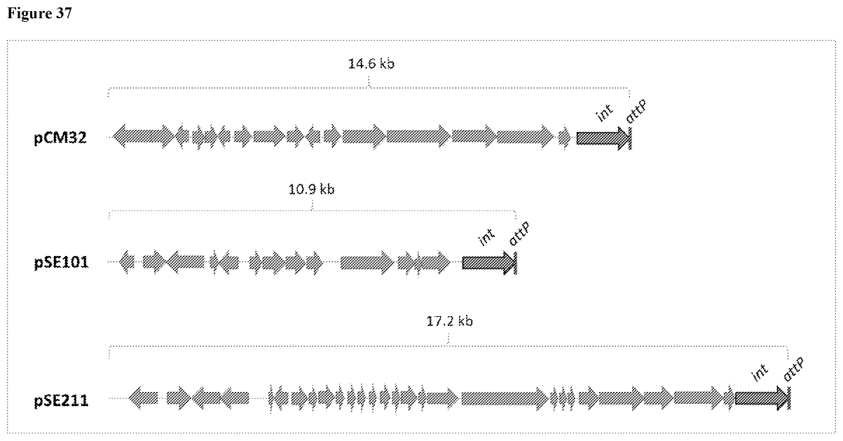

Vectors for integration of sequence into S. spinosa using pCM32, pSE101 and pSE211 are described in FIG. 37. Similarly, vectors using pSE101 homolog or pSE101 homolog of S. spinosa can also be constructed. These vectors will be tested to investigate their ability of integrating exogenous DNA into the genome of S. spinosa.

S. spinosa strains containing integrated exogenous DNA generated by the method described in the present disclosure can be used as a basis to improve strain performance in Saccharopolyspora spinosa. For example, such strains can be combined with the SNP Swap Library, the Promoter Swap Library, and/or the Terminator Library described in the above examples in a HTP system to create new S. spinosa strains having improved production of desired products, such as spinosyns.

The integrase systems described in Table 10 were tested and did not work. Inventors will test modified systems and other integrase system.

### Example 12. Origins of Replication for Self-Replicating Plasmid Systems for Saccharopolyspora spinosa

In the present example, origins of replication and replicative elements (e.g., genes encoding enzymes required for plasmid replication) are provided. These genetic elements may provide replication functionality in S. spinosa, thus they may enable the construction of a self-replicating plasmid system for S. spinosa. A self-replicating plasmid system would enhance the types of genetic engineering and screening that can be performed in this host.

One important molecular genetic tool currently lacking for S. spinosa is a self-replicating plasmid system. A plasmid system would expand the engineering capacity of S. spinosa in numerous ways. For example, it could (1) eliminate the need for successful integration by homologous recombination for testing metabolic engineering designs (e.g., gene duplications or heterologous enzymes could be introduced using the plasmid system to determine effects on host phenotype); (2) enable more rapid screening of libraries (genes, promoters, terminators, or ribosomal binding sites); (3) it would facilitate CRISPR-based genome editing by allowing the user to introduce CRISPR system components on and under control of the plasmid system.

Other plasmids from closely related species, including pWHM4, a self-replicating plasmid used extensively in S. erythraea (Vara et al., 1989, “Cloning of genes governing the deoxysugar portion of the erythromycin biosynthesis pathway in Saccharopolyspora erythraea (Streptomyces erythraeus”. J. Bacteriol. 171, 5872-5881) and pIJ101, a multi-copy broad host-range plasmid from Streptomyces lividans (Kieser et al., 1982, “pIJ101, a multi-copy broad host-range Streptomyces plasmid: functional analysis and development of DNA cloning vectors.” Mol Gen Genet 185:223-228) have been investigated for use in S. spinosa, but to our knowledge have not been used successfully.

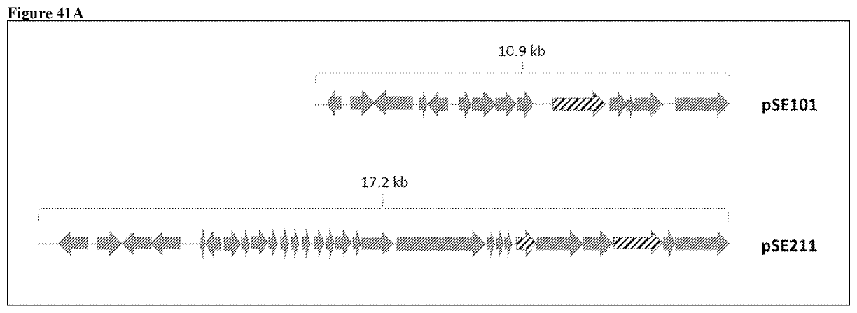

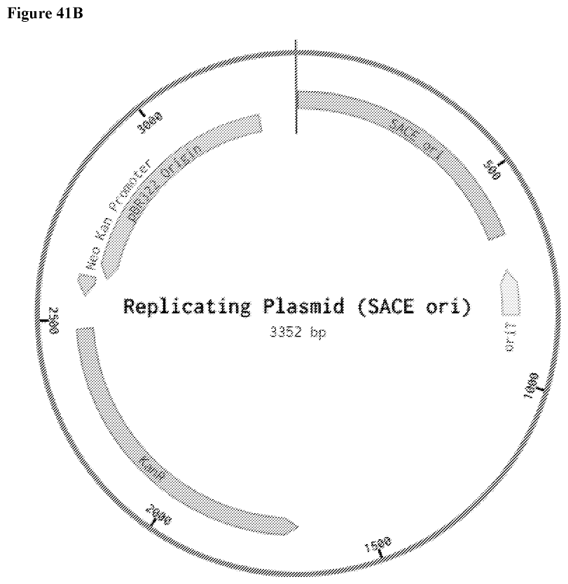

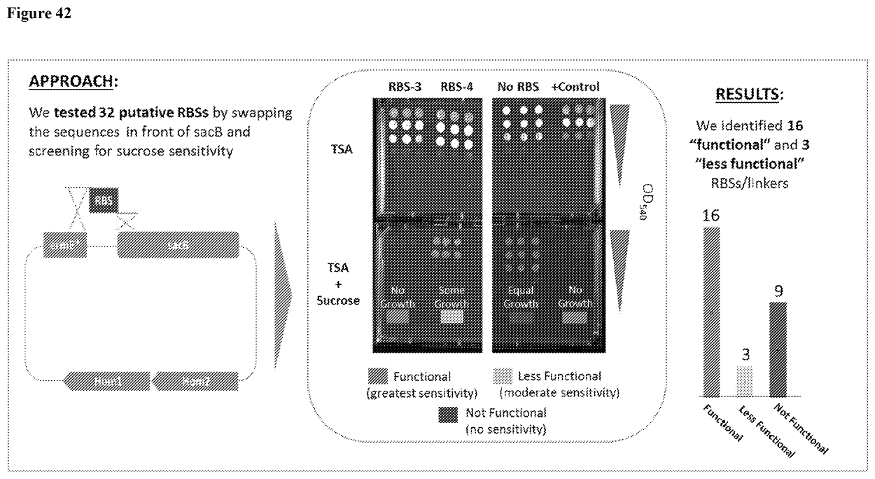

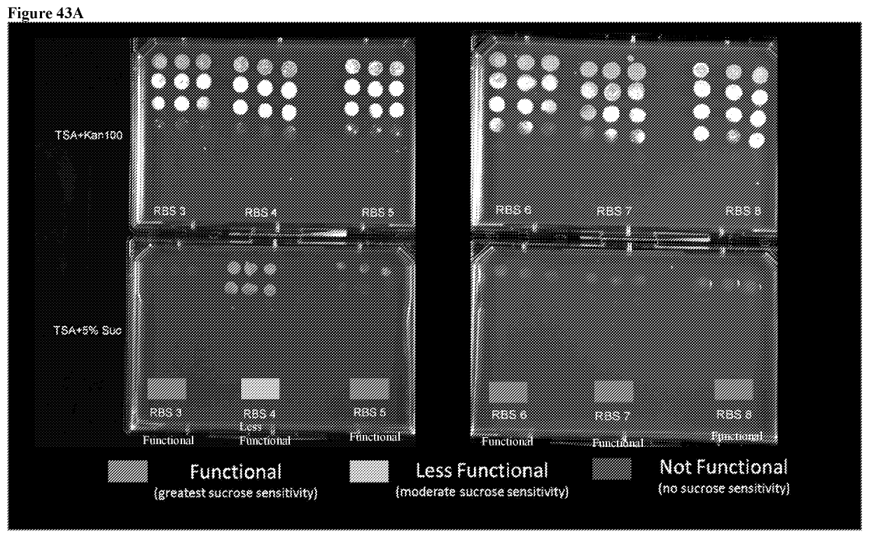

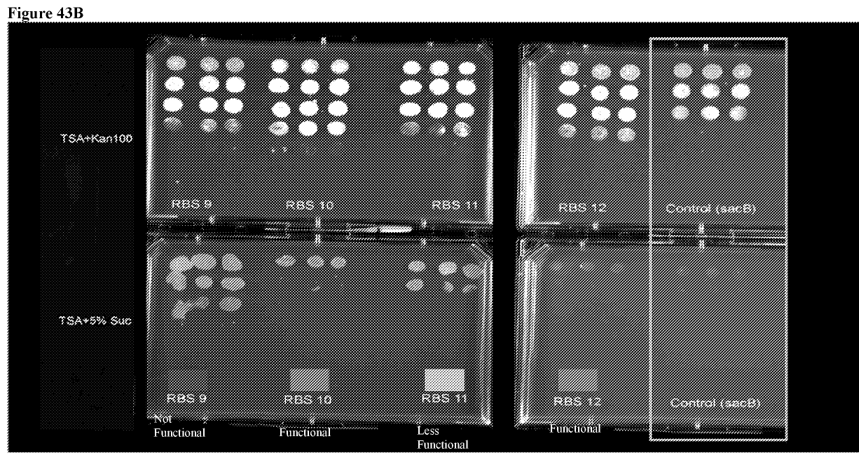

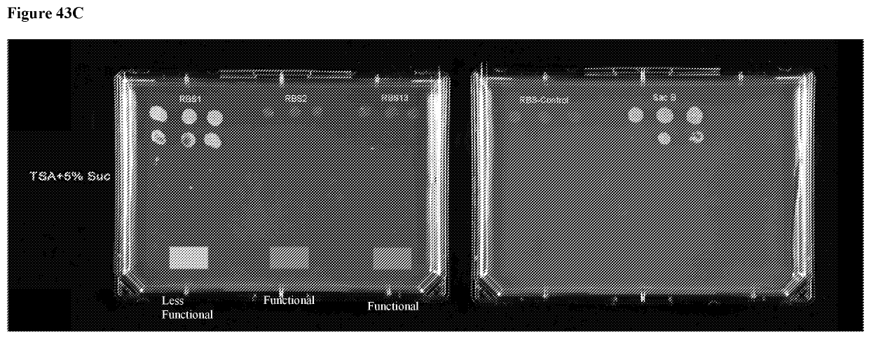

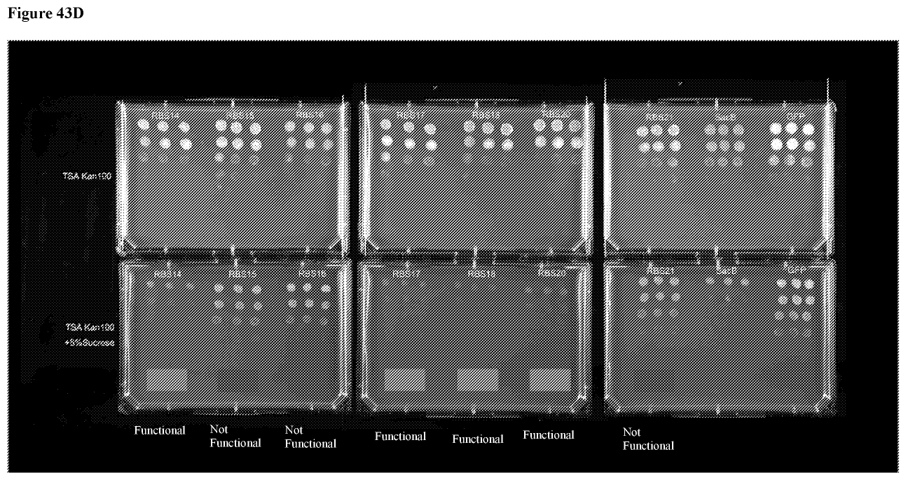

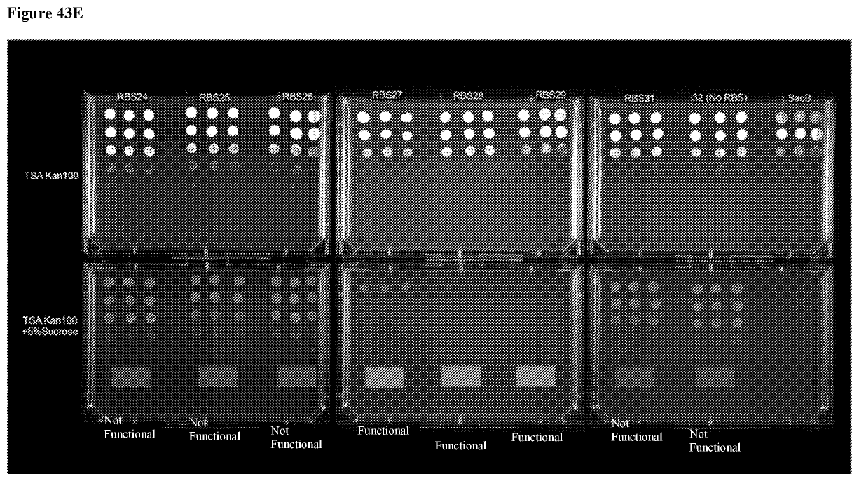

In some embodiments, sources of origins of replication include the putative chromosomal origin of replication found in S. erythraea, and Actinomycete Integrative and Conjugative Elements (AICEs) in plasmids pSE101 and pSE211 from S. erythraea (Te Poele et al., (2008) Actinomycete integrative and conjugative elements. Antonie Van Leeuwenhoek 94, 127-143), see FIG. 41A. Actinomycete Integrative and Conjugative Elements (AICES) are mobile genetic elements that are common in actinomycetes, including Saccharopolyspora spp. These elements can be found integrated in the genome or as autonomous, self-replicating plasmids.

To test these putative origins of replication, plasmids containing an antibiotic resistance marker and the putative origins of replication+/−other genes required for self-replication (e.g., in the case of AICEs) were assembled. The assembled plasmids were delivered to S. spinosa and antibiotic selection was used to select for transformants possessing the plasmid. PCR was used to confirm maintenance and stability of the plasmid. An exemplary plasmid is shown in FIG. 41B. These putative origins of replication were tested and did not work. Inventors will test modified designs and other putative origins of replication.

### Example 13. HTP Genomic Engineering—Implementation of a Ribosome Binding Site (RBS) Library to Improve Strain Performance in Spinosyns Production in Saccharopolyspora

Previous examples have demonstrated the power of the HTP strain improvement programs of the present disclosure for rehabilitating industrial strains. Examples 2 and 3 described the implementation of SNP swap techniques and libraries exploring the existing genetic diversity within various base, intermediate, and industrial strains.

This example illustrates embodiments of the HTP strain improvement programs using the Ribosomal Binding Site library techniques of the present disclosure.

### A. Identification of a Target for Applying RBS Library

Applying RBS library is a multi-step process that comprises a step of selecting a set of “n” genes to target.

The inventors have identified a group of potential pathway genes to modulate via the promoter ladder methods of the present disclosure. (See, Example 4 and FIG. 12A to FIG. 12D).

### B. Creation of RBS Library

A major goal of our genetic engineering efforts, and metabolic engineering more broadly, is to alter host metabolism, optimize biosynthetic pathways, and introduce or duplicate pathway genes in order to improve the yield of a desired product. Success relies on the ability to perturb and balance expression of genes both within (on-pathway) and outside (off-pathway) of the biosynthetic gene cluster or over-express non-native genes or copies of genes that are introduced. There are limited available genetic tools in S. spinosa, including characterized RBSs. This invention is a genetic engineering tool, which allows the design of multi-gene polycistronic operons for integration and the tuning of protein expression in S. spinosa.

Ribosomal binding sites (RBSs) are short sequences of nucleotides that are located upstream of the start codon on an mRNA transcript that is responsible for recruiting ribosomes and initiating translation of protein. Accordingly, they are important regulators of translation and protein expression. However, RBSs can also interact with nearby nucleotides in the 5′UTR, the promoter or coding region of a gene to influence rates of transcription and/or translation. Through these interactions and resulting secondary structure, ribosomal binding sites can “tune” expression of genes.

RBS libraries are common components of synthetic biology toolkits and have been developed for various organisms. In addition, tools have been developed for predicting synthetic RBSs that will interact favorably with a gene of interest (Salis et al., “Automated design of synthetic ribosome binding sites to control protein expression.” Nat Biotechnol. 2009; 27:946-950. doi: 10.1038/nbt.1568). However, this is the first such library and first native RBSs described and characterized for S. spinosa.

To identify putative native RBSs, the nucleotide sequences upstream from the START codon or intergenic regions between genes in polycistronic operons were selected. RBSs were selected for genes expected to be highly expressed, based on proteomic data from the literature (Luo et al., “Comparative proteomic analysis of Saccharopolyspora spinosa SP06081 and PR2 strains reveals the differentially expressed proteins correlated with the increase of spinosad yield.” Proteome SCI. 2011, 9: 1-12), or for genes related to spinosyn production. Predictions were based on annotations available in the PATRIC database (https://www.patricbrc.org/) at the time of analysis. RBSs were assayed using a counterselectable marker (sacB)-level of growth on selective media constituted a metric for functionality.

In this example, the inventors have created a library of a group of 19 ribosomal binding sites (RBSs) with varying degrees of translational activity for use in S. spinosa and related hosts. The library is comprised of synthetic sequences previously described in different hosts and sequences native to S. spinosa that have not previously been characterized:

TABLE 11
Summary of the RBS sequences,
their source, size, and relative function

| | Gene | RBS seq | (bp) | Function |
|---|---|---|---|---|
| RBS1 | PermE* | aggaggtcccat | 12 | + |
| | | (SEQ ID NO. 97) | | |
| RBS2 | spnA (polyketide synthase | ccaggaatcggagggg | 25 | ++ |
| | loading & extender module1) | cagtaccga | | |
| | | (SEQ ID NO. 98) | | |
| RBS3 | spnC (polyketide synthase | gcaacttcctggaggg | 25 | ++ |
| | extender modules 3-4) | aaacgccac | | |
| | | (SEQ ID NO. 99) | | |
| RBS4 | spnO (putative NDP-hexose- | tcgtcacggcagtgag | 25 | + |
| | 2,3-dehydratase) | ggattgggc | | |
| | | (SEQ ID NO. 100) | | |
| RBS5 | gdh (gdh (dTDP-glucose | cgaaatcccggcgagg | 25 | ++ |
| | 4,6-dehydratase)) | aagggcgcg | | |
| | | (SEQ ID NO. 101) | | |
| RBS6 | linker_A (aldehyde | cgcctcggcccccttc | 28 | ++ |
| | dehydrogenase, AldA) | aggaggagacag | | |
| | | (SEQ ID NO. 102) | | |
| RBS7 | linker_B (acetolactate | ctccagacgcccacgc | 26 | ++ |
| | synthase) | aaggagaccc | | |
| | | (SEQ ID NO. 103) | | |
| RBS8 | linker_C | actagtaaggaggtcc | 19 | ++ |
| | | aac | | |
| | | (SEQ ID NO. 104) | | |
| RBS9 | linker_D | aagaggtatatatta | 15 | - |
| | | (SEQ ID NO. 105) | | |
| RBS10 | gtt (Glucose-1-phosphate | ccaccgctggaggtat | 20 | ++ |
| | thymidylyltransferase 1) | ccgg | | |
| | | (SEQ ID NO. 106) | | |
| RBS11 | TDH (Glyceraldehyde-3- | aggagagatcggc | 13 | + |
| | phosphate dehydrogenase) | (SEQ ID NO. 107) | | |
| RBS12 | BioBrick_1 | aaagaggagaaa | 12 | ++ |
| | | (SEQ ID NO. 108) | | |
| RBS13 | BioBrick_2 | attaaagaggagaaa | 15 | ++ |
| | | (SEQ ID NO. 109) | | |
| RBS14 | GroES (Molecular chaperon | agaaggtggaggtcac | 19 | ++ |
| | GroES) | acc | | |
| | | (SEQ ID NO. 110) | | |
| RBS15 | GroEL (Molecular chaperon | aagggctgttggaatc | 16 | - |
| | GroEL) | (SEQ ID NO. 111) | | |
| RBS16 | IF-1 (Translation initiation | attgaggtcgagggtc | 18 | - |
| | factor IF-1) | gg | | |
| | | (SEQ ID NO. 112) | | |
| RBS17 | XNR_1700 (Periplasmic | ggcggtgaatgatccg | 22 | ++ |
| | murein peptide-binding | ccgcgc | | |
| | protein precursor) | (SEQ ID NO. 113) | | |
| RBS18 | S20 (30 s ribosomal | gacgaggaagaggcgc | 20 | ++ |
| | protein S20) | caca | | |
| | | (SEQ ID NO. 114) | | |
| RBS19 | S12 (ribosomal protein S12) | acgttacgctcgtcgc | 15 | NA |
| | | (SEQ ID NO. 115) | | |
| RBS20 | S12 (ribosomal protein S12) | gggacgttacgctcgt | 19 | ++ |
| | | cgc | | |
| | | (SEQ ID NO. 116) | | |
| RBS21 | DnaK (Hsp 70) | tcgtgacctcggtgct | 21 | - |
| | | gaaca | | |
| | | (SEQ ID NO. 117) | | |
| RBS22 | elongation factor Tu | aggaggaacaatcca | 15 | NA |
| | | (SEQ ID NO. 118) | | |
| RBS23 | F0F1 ATP synthase subunit | ccgcaggaagtgagtg | 18 | NA |
| | beta | ac | | |
| | | (SEQ ID NO. 119) | | |
| RBS24 | molecular chaperone DnaK | cgtgacctcggtgctg | 20 | - |
| | | aaca | | |
| | | (SEQ ID NO. 120) | | |
| RBS25 | phage shock protein A, | aattcccggggatcta | 18 | - |
| | PspA | cc | | |
| | | (SEQ ID NO. 121) | | |
| RBS26 | 2-oxoglutarate | cgaggcgaacgcagcc | 17 | - |
| | decarboxylase | (SEQ ID NO. 122) | | |
| RBS27 | 5-methyltetrahydropteroyl- | gcgaaggagagccccc | 16 | ++ |
| | triglutamate homocysteine | (SEQ ID NO. 123) | | |
| | methyltransferase | | | |
| RBS28 | 50 S ribosomal protein | ccgaaaggaacgccga | 17 | ++ |
| | L7/L12 | c | | |
| | | (SEQ ID NO. 124) | | |
| RBS29 | DNA-directed RNA polymerase | gaggaaaggaaaacga | 17 | ++ |
| | subunit alpha | a | | |
| | | (SEQ ID NO. 125) | | |
| RBS30 | 30 S ribosomal protein S5 | gaacggaagggacgcc | 18 | NA |
| | | tg | | |
| | | (SEQ ID NO. 126) | | |
| RBS31 | DnaK (6929) | cggcgggtcggagagg | 21 | — |
| | | agtgc | | |
| | | (SEQ ID NO. 127) | | |
| RBS32 | Negative_1 (ermE only) | — | 0 | |

Note:
The library contains 26 native RBSs (those w/ associated Gene IDs). The remaining five sequences come from synthetic or heterologous sources.
(-) = not functional;
(+) = less functional;
(++) = functional
“NA” indicates RBSs for which we do not have data

Thus, the present disclosure provides a diverse library of functional RBS sequences that are required as spacers between genes in multi-gene, polycistronic integrations. The sequence diversity and variation in strengths these RBSs provides an opportunity to use these to tune expression of genes up or down by inserting different RBSs between promoters and genes.

C. Associating RBS from the Library with Target Genes

Another step in the implementation of a RBS libraries is the HTP engineering of various strains that comprise a given RBS from the RBS library associated with a particular target gene.

If a native RBS exists in front of target gene n and its sequence is known, then replacement of the native RBS with each of the RBSs in the library can be carried out. When the native RBS does not exist or its sequence is unknown, then insertion of each of the RBS in the library in front of gene n can be carried out. In this way a library of strains is constructed, wherein each member of the library is an instance of a RBS operably linked to n target, in an otherwise identical genetic context.

### D. HTP Screening of the Strains

A final step in the applying the RBS library is the HTP screening of the strains in the aforementioned library. Each of the derived strains represents an instance of a RBS linked to n target, in an otherwise identical genetic background.

By implementing a HTP screening of each strain, in a scenario where their performance against one or more metrics is characterized, the inventors will be able to determine what RBS/target gene association is most beneficial for a given metric (e.g. optimization of production of a molecule of interest).

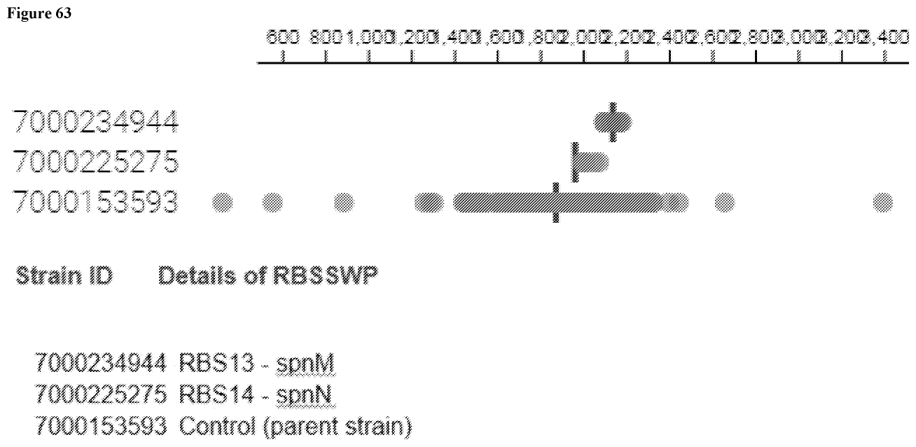

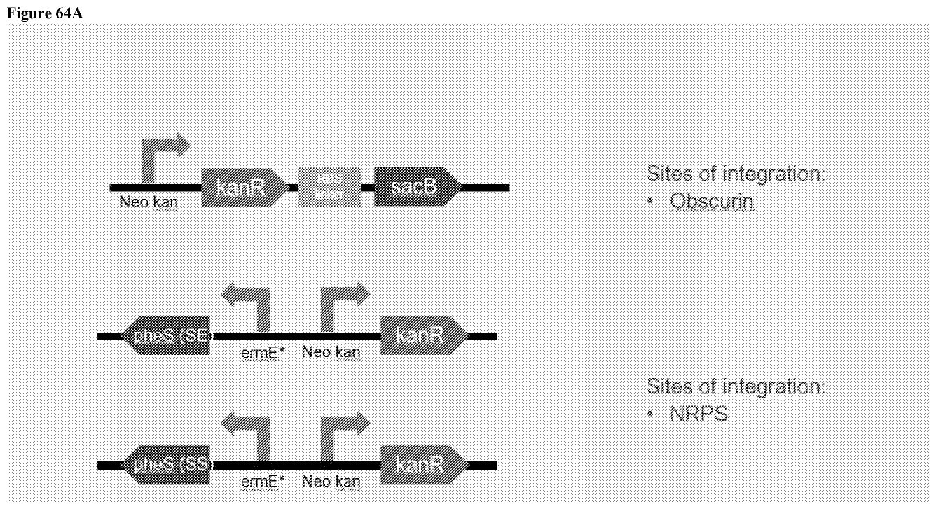

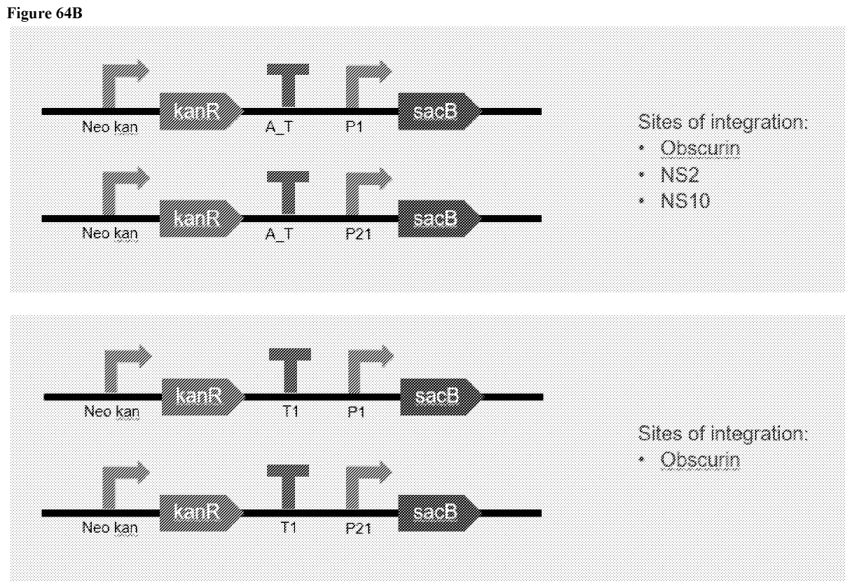

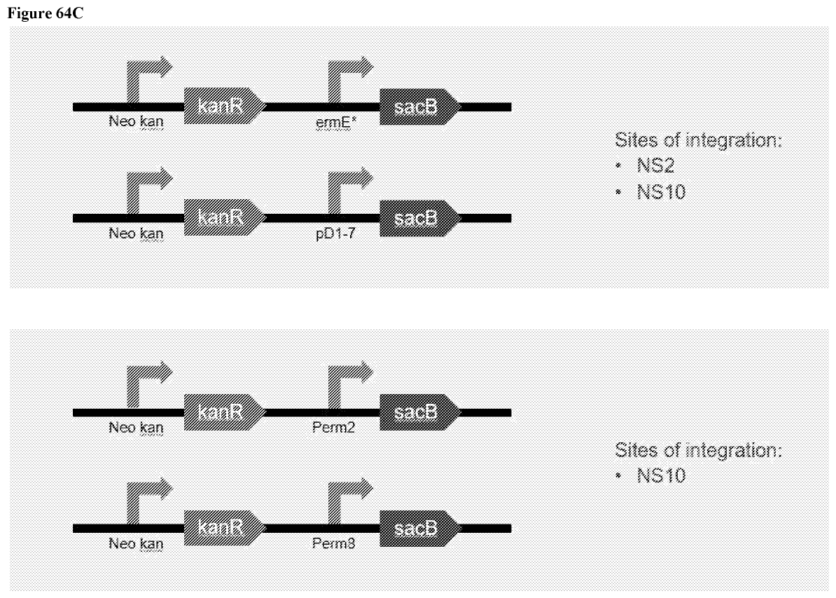

Data demonstrating the utility of this engineering approach is shown in FIG. 63. Ribosome binding sites were inserted upstream of a number of targeted genes to modify translational efficiency, and these engineered strains were tested in comparison to parent strain in a plate assay for polyketide productivity. Several “RBS swap” strains exhibited an improvement compared to the parent strain.

### Example 14—HTP Genomic Engineering—Implementation of a Transposon Mutagenesis Library to Improve Strain Performance in Saccharopolyspora

This example describes a method to produce strain libraries by in vivo transposon mutagenesis in S. spinosa. Resulting libraries can be screened to identify strains that exhibit improved phenotypes (e.g. titer of a specific compound, such as spinosyns). Strains can be further used in rounds of cyclical engineering or to decipher genotypes that contribute to strain performance. Strains in the library can also be used for consolidation with other strains having different genetic perturbation(s) for creation of improved strain having increased production of one or more desired compounds, similar to SNP Swap Library used in Example 3 above.

Thus, the present disclosure describes a method of using an EZ-Tn5 Transposome system (Epicenter Bio) in S. spinosa to create a transposon mutagenesis microbial strain library. The transposase enzyme can be first complexed with a DNA payload sequence flanked by mosaic element (ME) sequences and the resulting protein-DNA complex can be transformed into cells. This will result in the random integration of the DNA payload into the organism's genomic DNA. Depending on the payload to be introduced, either Loss-of-Function (LoF) libraries or Gain-of-Function (GoF) libraries can be produced.

Loss-of-Function (LoF) transposon libraries—The sequence of the payload may be varied to elicit diverse phenotypic responses. In the basal case of a loss-of-function (LoF) library, this payload comprises a marker that allows for the selection of successful transposon integration events.

Random loss-of-function mutations can be made in vivo in a microorganism using an Tn5 transposase system (EZ-Tn5; EpiCentre®). The EZ-Tn5 transposase system is stable and can be introduced into living microorganisms by electroporation. Once in the cell, the transposon system is activated by Mg2+ in the host cell and the transposon is randomly inserted into the host's genomic DNA.

Gain-of-Function (GoF) transposon libraries—To create GoF libraries, more complex incarnations of the genetic payload build upon the basal case, by incorporating additional features such as promoter elements, solubility tags (in this case, called Gain-of-Function solubility tag transposon), and/or counter-selectable markers to facilitate loop-out of a portion of the payload containing the selectable marker thus allowing serial transposon mutagenesis (in this case, called Gain-of-Function recyclable transposon). Together these implementations enable the creation of diverse libraries to improve a host phenotype.

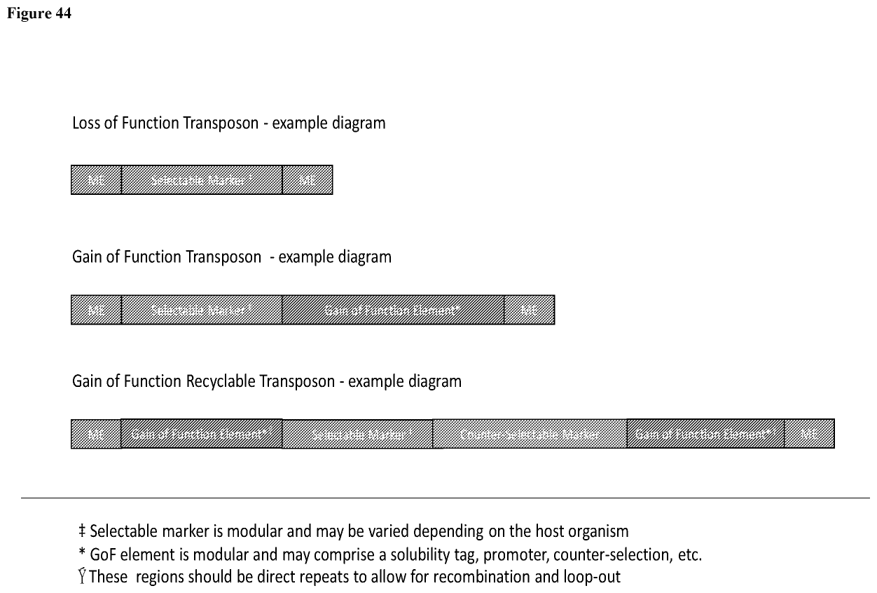

Non-limiting exemplary constructs for transposons of the present disclosure are shown in FIG. 44, and the sequences of representative Loss-of-Function (LoF) transposon, Gain-of-Function (GoF) transposon, Gain-of-Function recyclable transposon, and Gain-of-Function solubility tag transposon are provided as SEQ ID No. 128, SEQ ID No. 129, SEQ ID No. 130, and SEQ ID No. 131, respectively. These transposons can be complexed with transposase and transformed into cells. The resulting cells will have random integration of the DNA payload, thus forming transposon mutagenesis microbial strain libraries. The libraries can be further screened according to the HTP procedure described herein and evaluated for phenotype improvements. Strains with desired phenotypes due to the transposon integration can be isolated for further characterization, and further engineering, according to any method described in the present disclosure.

For example, LoF transposon libraries and GoF transposon libraries can be screened against the parent strains, and the performance data (titer of spinosyn) can be analyzed. Some of the new strains created in these libraries will have improved performance compared to the parent strain.

Methods described herein solve two main problems. First, even in a well studied organism, large portions of the genomic landscape remain poorly understood. It has also been noted that well-understood genetic elements may interact in unexpected ways. To this end, the present disclosure provides effective genetic engineering method for elicitation of phenotypic perturbations. Second, with slow growing or genetically recalcitrant organisms—especially those with large genomes—it maybe be time or cost prohibitive to perform targeted genetic perturbations on all possible genetic targets. The present disclosure provides an effective way to create strains with perturbed genome, which may lead to improved performance in producing a desired compound in the strain. Thus, the present disclosure addresses these problems, by a method for readily and randomly modulating genetic elements of host organisms using in vivo transposon mutagenesis. In this manner, strain libraries that harbor different mutations (gain-of-function and loss-of-function) can be made very quickly and can implicate new genetic targets to further improve a host's phenotype.

### Example 15. Neutral Integration Sites for the Insertion of Genetic Elements in Saccharopolyspora

Engineering gene duplications and refactored biosynthetic pathways in S. spinosa is limited by the number of known neutral integration sites that have been characterized for this host. It is likely that several neutral sites exist within the S. spinosa genome, but, to date, only one neutral integration site has been characterized. This particular site, obsA (US20100282624, incorporated by reference in its entirety), has been previously reported, but the lack of additional sites constrains our ability to make multiple, serial genetic changes. Additional neutral integration sites would enhance the capacity, and speed at which we are able, to engineer and test multiple combinatorial gene integrations.

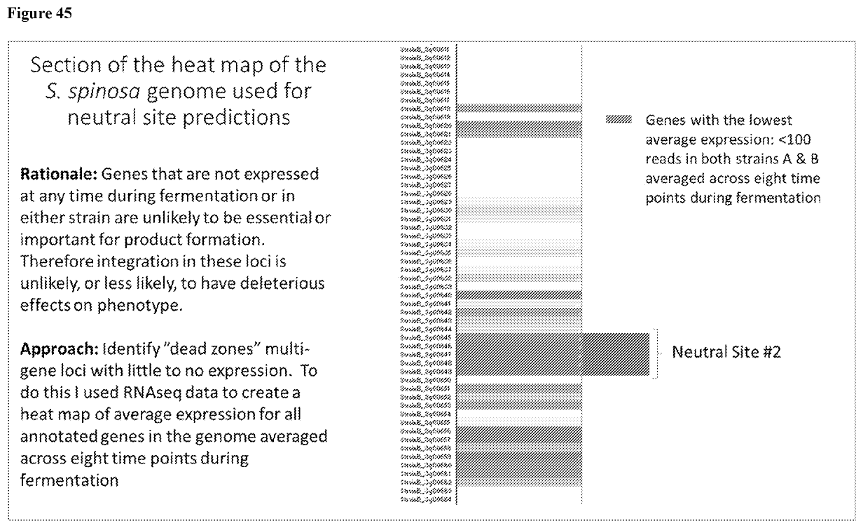

RNAseq data (a replicated time series sampled during fermentation and comparing expression in two strains), was used to identify multi-gene loci with little or no expression in either strain or at any time point during fermentation. The guiding rationale was that genes not expressed at any time during fermentation or in either strain are unlikely to be essential or important for production (see FIG. 45). Therefore integration into these loci is unlikely, or less likely, to have deleterious effects on phenotype. Once these sites were identified, the loci were located within the reference genome and integration constructs were designed to introduce a single base-pair mutation in the center of the site.

Thus, the present disclosure provides a set of neutral integration sites—e.g., genetic loci into which individual genes or multi-gene cassettes can be stably and efficiently integrated within the genome of S. spinosa by conjugation and homologous recombination. To be deemed a neutral site, genetic integration of a payload will show limited effects on growth and predictable levels of expression. The sites we have identified and are currently exploring include eleven loci that are dispersed throughout the genome. Each site has the potential to add expand our genetic engineering capacity by yielding an integration site to integrate genetic payloads. The number of sites available is proportional to the number of factors that we can include in full-factorial, combinatorial gene integration designs and thus enhances our engineering capacity. These sites are summarized in the Table 12 below.

TABLE 12
Summary of eleven putative neutral integrations sites,
associated genes, the mutations introduced and integration
efficiency - colony-forming units (CFUs) for each parent
strain.

CFUs

| Neutral Site | SEQ ID No. | Mutation | A | B |
|---|---|---|---|---|
| 1 | 132 | C:G | 88 | 47 |
| 2 | 133 | T:A | 50 | 33 |
| 3 | 134 | C:G | 91 | 17 |
| 4 | 135 | C:G | 67 | 28 |
| 5 | 136 | G:C | 16 | 0 |
| 6 | 137 | G:C | 129 | 18 |
| 7 | 138 | C:G | 84 | 32 |
| 8 | 139 | T:A | — | — |
| 9 | 140 | A:T | 94 | 25 |
| 10 | 141 | C:G | 55 | 41 |
| 11 | 142 | A:T | — | — |

Annotation refers to the gene in the center of the neutral site into which we introduced the indicated mutation.
CFUs indicate the number of colonies (ex-conjugants) counted in a single well of a divided (48well) Q-Tray
* Conjugations are in progress (results are pending)

The sites are located within multi-gene loci for which little to no expression (transcription; mRNA) is observed. They were identified using a time series of RNAseq data comparing gene expression in two different strains.

To evaluate integration efficiency, a single nucleotide polymorphism was introduced into the center of each site. Conjugation efficiency is reported for each site in Strain A and B (Table 12).

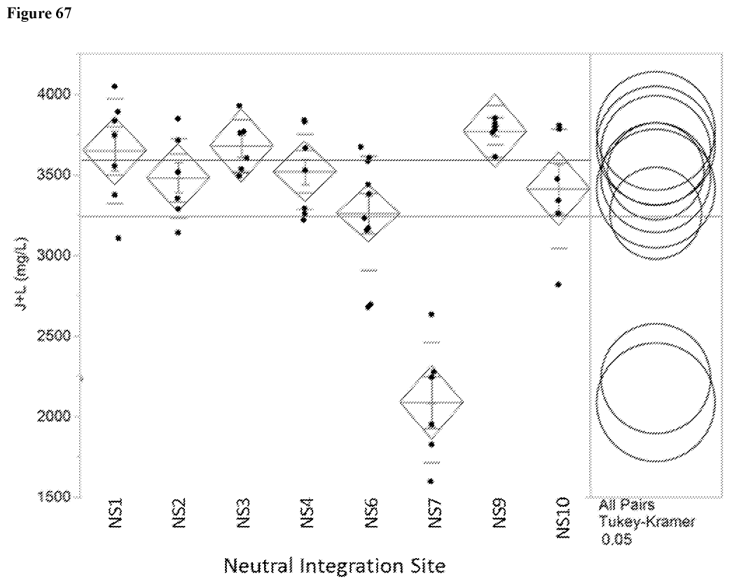

The resulting strain B-derived strains were evaluated for product titer, relative to the strain B parent (FIG. 67). Product titer (spinosyns J+L) of strain B-derived strains with SNPswap payloads integrated at the indicated neutral site was analyzed. Strains with integration at sites 1, 2, 3, 4, 6, 9 & 10 have similar product titers and do not differ from the expected titer (i.e., average titer of strain B; higher bar on the figure). Integration at neutral site 7 appears to have a negative impact on product titer.

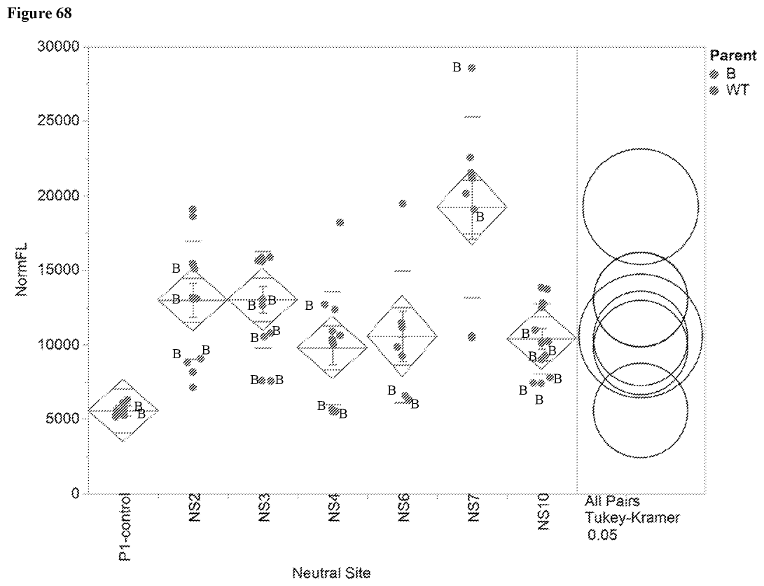

To further evaluate these sites and compare the expression of integrated payloads, we evaluated the expression of a fluorescent reporter (SEQ ID No. 81) under control of a strong promoter (SEQ ID No. 25) following integration at each site in both strains A (WT) and B (FIG. 68). Expression is similar at most sites. Only NS7 was significantly different from other neutral sites we evaluated (NS2, NS3, NS4, NS6, and NS10).

### Example 16. HTP Genomic Engineering—Implementation of an Anti-Metabolite Selection/Fermentation Product Resistance Library to Improve Strain Performance in Saccharopolyspora

This example illustrates embodiments of creating anti-metabolite selection/fermentation product resistance libraries for generating genetic diversity in Saccharopolyspora and methods of using such libraries for HTP genetic engineering.

In order to improve production of desired compounds by microbes it is often needed to bypass forms of molecular regulation that are not immediately amenable to rational engineering. Examples include end-product inhibition by different pathways. In this example we subjected S. spinosa to either anti-metabolites (alpha-methyl methionine) or fermentation products (spinosyn J/L) and isolated colonies that have improved growth under these conditions. High-throughput screening of such colonies identified isolates that have better fermentation performance in plate model as compared to parent strain, which indicated that the strategies are potentially useful for improving strain performance.

Microbes produce a variety of compounds as a part of the fermentation process. Sometimes the accumulation of such compounds severely inhibits the growth and physiology of the microbes. Ethanol production is an example of growth inhibition (toxicity) by the fermentation product. At molecular level, the products of pathways can often inhibit the enzymes responsible for their production in effort to minimize waste. While this is beneficial for microbial evolution and survival, these feedback mechanisms can severely hamper industrial fermentation (Fermentation Microbiology and Biotechnology, Third Edition, ISBN 9781439855799), where the goal is to radically increase flux through certain pathways and buildup of product. To improve fermentation and lengthen the time during which the microbe can synthesize the desired metabolites is need to overcome a) the potential toxicity of the end product, and b) feed-back inhibition of molecular pathways needed for the formation of the desired end-product.

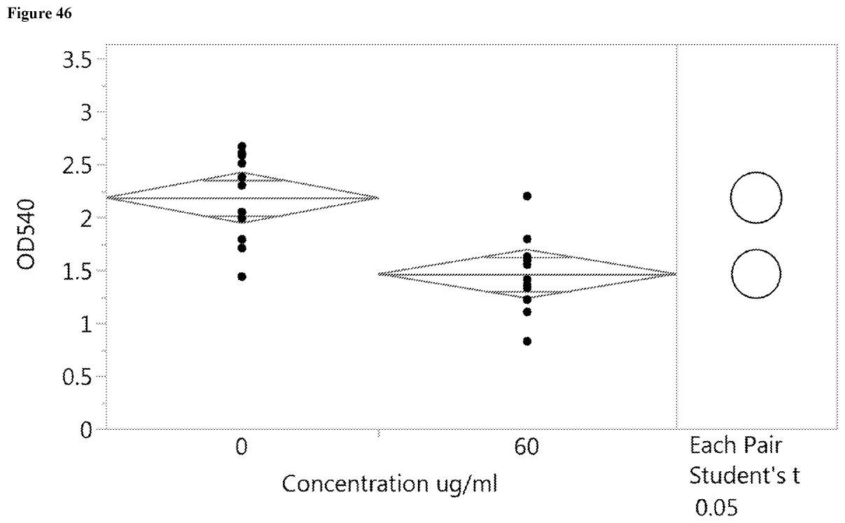

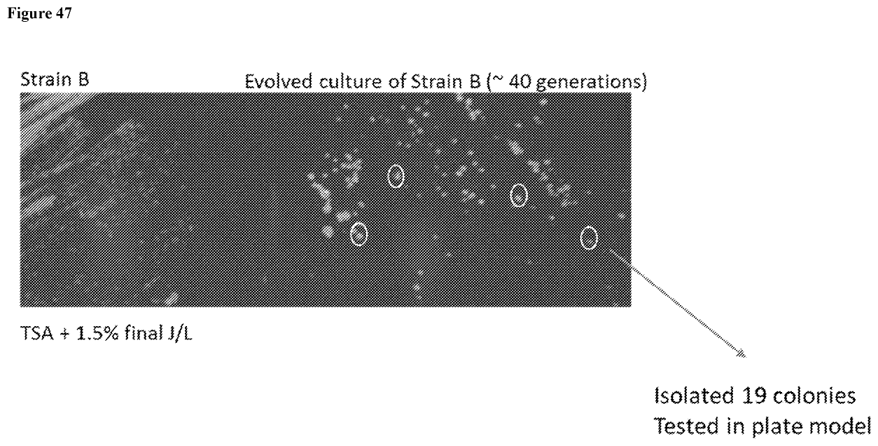

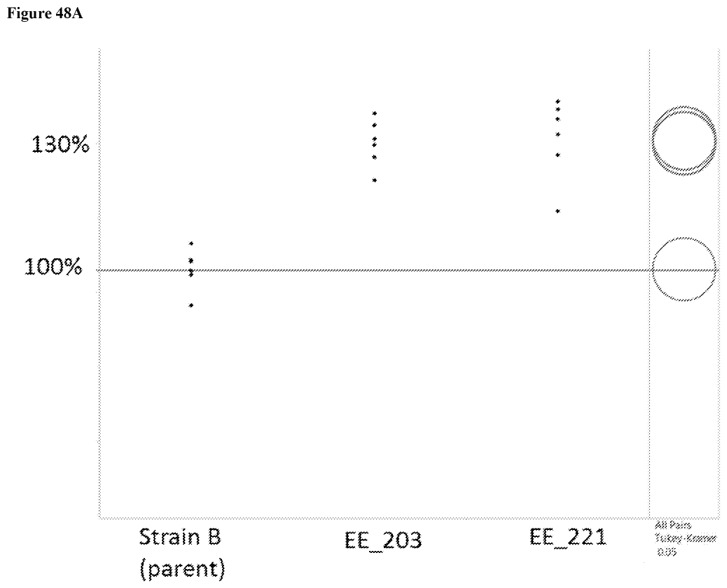

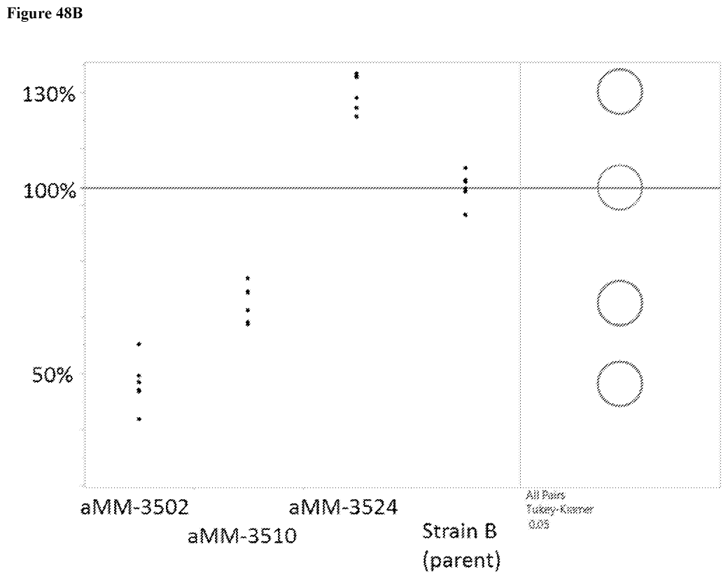

S. spinosa growth is sensitive to the presence of its fermentation product. (FIG. 46). We hypothesized that if we improved its tolerance to product we may improve the strain productivity. Therefore the steps outlined below were undertaken to select strains better capable of surviving the fermentation product. We isolated more resistant strains (FIG. 47). Interestingly two of the isolates performed much better against spinosyn J/L than the parent in a plate model for spinosyn production (FIG. 48A). We also isolated one strain performed much better against metabolite alpha-Methyl-methionine (aMM) than the parent in a plate model for spinosyn production (FIG. 48B).

Spinosyn production required NADPH and SAM as co-factors. As either of those can be limiting to spinosyn formation, and each can inhibit enzymes responsible for their respective synthesis, we sought ways remove feedback inhibition by SAM. In E. coli SAM can inhibit the MetA protein, which is responsible for the synthesis of precursors to SAM. The typical approach in E. coli has been to grow strains in the presence of the anti-metabolite alpha-Methyl-methionine (aMM), which selects for metA mutants that are insensitive to feed-back regulation (Ususda and Kurahashi, 2005, Appl Env. Micro, June 2005, p 3228-3234). There is no clear metA homologue in S. spinosa, but since S. spinosa is sensitive to aMM, we took a similar approach and selected resistant mutants, hoping that they have increased SAM accumulation and maybe better spinosyn production. In order to improve production of desired compounds by microbes, it is often needed to bypass forms of molecular regulation that are not immediately amenable to rational engineering. Examples include end-product inhibition by different pathways.

Particularly, we subjected parent S. spinosa strains to either anti-metabolites (e.g., alpha-methyl methionine) or fermentation products (e.g., spinosyn J/L). We first determined sensitivity of S. spinosa to selection agent and the conditions and media for the experiment. Without proper starting point in terms of concentrations used, the experiments may fail completely. For the spinosyn J/L experiment it took several weeks and multiple attempts to find a concentration that inhibited growth but that did not kill the cells. The solution was a combination of adjusting spinosyn concentration and the amount of biomass used for inoculation. aMM requires the use of minimal media, which we had to identify and validate first before we could proceed with selection. This step lays the foundation for a successful selection strategy. The minimal media has the composition listed below:
Ingredients (per 1 L):
Starch, soluble 10.0 g
Dipotassium phosphate 1.0 g
Magnesium sulphate. heptahydrate 1.0 g
Sodium chloride 1.0 g
Ammonium sulphate 2.0 g
Calcium carbonate 2.0 g
Ferrous sulphate, heptahydrate 0.001 g

Once the concentrations for selection were determined, select for more resistant isolates in the conditions described above was carried out. For selection, multiple passaging of cultures was needed for selection in liquid (7 passages, ˜40 generations). Multiple independent cultures were maintained in parallel to increase the chance of independent mutation events which satisfy the imposed selection. The duration and frequency of each passage can be empirically determined. The selection strategy determines what traits are selected for. Poor design can result in selecting for strains that would not perform well under desired industrial conditions. A good alignment and/or mitigation strategies (secondary screens) will be need to improve the success of the selection. An example of selection of strains in the presence of spinosyn J/L was demonstrated in FIG. 47. Selected strains clearly grew better than the parent in the presence of spinosyn J/L.

Selected strains were further validated to demonstrate that these isolates are indeed more resistant than parent strains. This validation of the selection is a good indicator that the strategy is working and may be used as a decision point of when to proceed to the next step.

Next, selected strains were further analyzed by HTP screening to determine if the selected characteristics are beneficial for the desired industrial process. Since the cells can solve a particular selection challenge in many ways, most of which may not be of industrial interest, the HTP screening is a crucial step in identifying the isolates that are to be further characterized and used for consolidation. From our first studies it appears that only ˜2-5% of selected isolates are of interest. An example of selected strains that have better performance than parent in HTP plate fermentation model is shown in FIG. 48A (spinosyn J/L) and FIG. 48B (aMM).

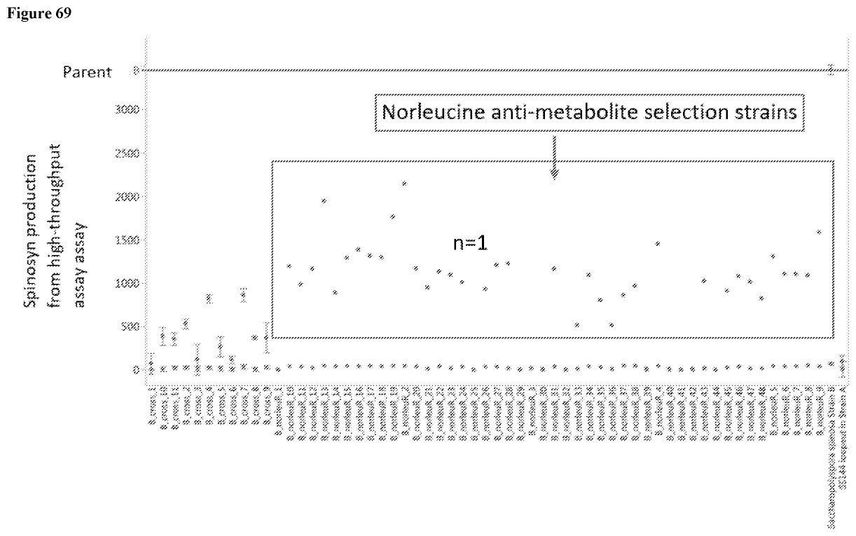

Optionally, mutations that caused the improved performance in the selected strains can be identified and associated sequence can be isolated. This will facilitate consolidating these mutations into other desired strains as described herein. An initial test result is shown in FIG. 69.

### Example 17. HTP Genomic Engineering—Use of sacB or pheS as Counterselection Markers in S. spinosa for the Generation of Scarless Mutant Strains

This example illustrates embodiments of creating “scarless” mutant Saccharopolyspora strains using sacB or/and pheS as counterselection markers.

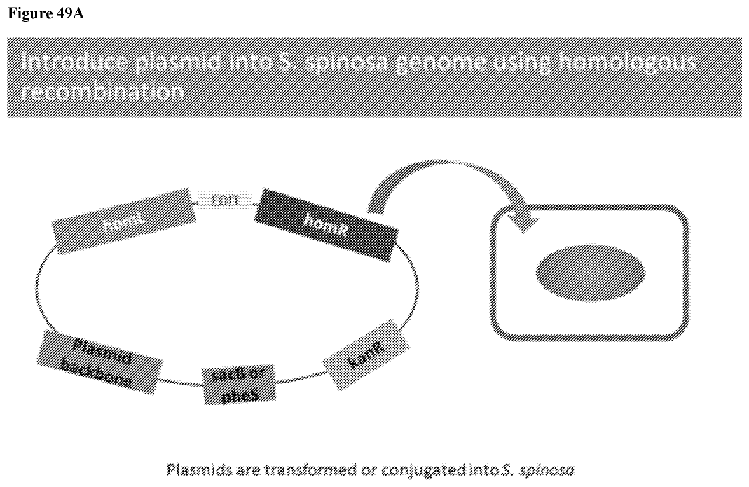

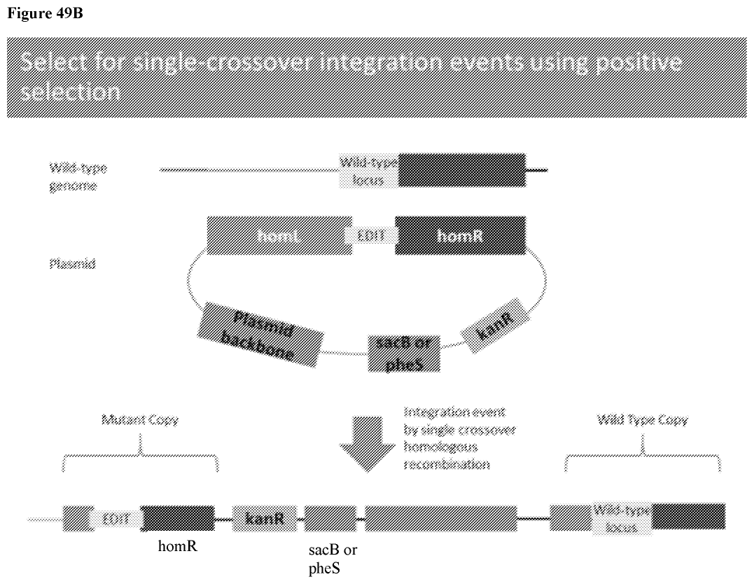

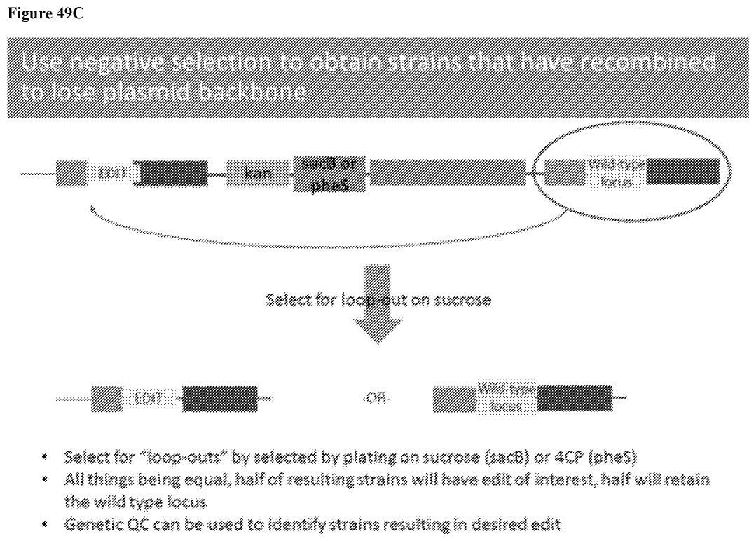

Previously described in the art, US20170101659A1 discusses engineering polyketide producing strains for improved productivity at the polyketide synthase gene loci using tools such as temperature sensitive origins of replication and selection markers. The detailed requirements and constraints of this methodology (including relying on the repetitive nature of the PKS coding regions) as well as the limited additional examples in the art illustrate the challenges of engineering industrially-relevant microbes like S. spinosa. However, precise genome editing at any location in the genome is important to be able to make intended modifications in the S. spinosa host strain including for the application of improving a phenotype of an organism. Additionally, resistance marker recycling enables stacking genetic modifications in a single strain where limited resistance markers exist, and is also important for facilitating registration of these microbes in manufacturing applications (i.e. antibiotic resistance-free). In the present example, we demonstrate the use of sacB and/or pheS as counterselection markers together with homology arms used to target any location in the genome to enable scarless, markerless directed genome editing (see FIG. 49A to FIG. 49C).

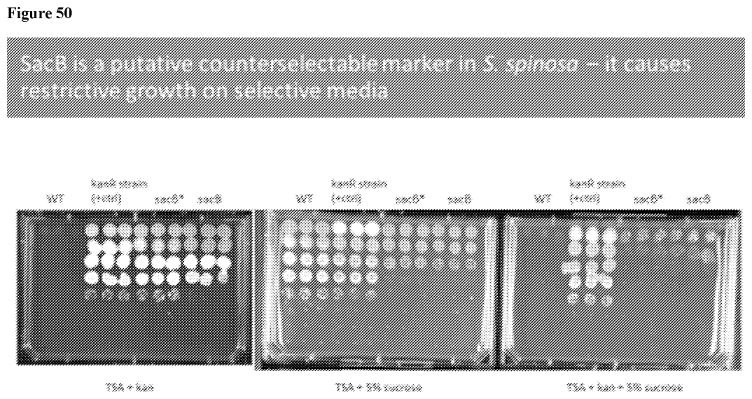

The sacB gene encodes a levansucrase that converts sucrose to levans, which are known to be toxic to many microbes (Reyrat et al., “Counterselectable Markers: Untapped Tools for Bacterial Genetics and Pathogenesis”, Infect Immun. 1998 September; 66(9): 4011-4017, and Jäger et al., “Expression of the Bacillus subtilis sacB gene leads to sucrose sensitivity in the gram-positive bacterium Corynebacterium glutamicum but not in Streptomyces lividans.”, J Bacteriol. 1992 August; 174(16):5462-5). In absence of sucrose, carriers of the sacB gene grow in a healthy manner, in presence of sucrose only strains that have lost the sacB gene can survive. This concept has heavily been used in many gram-negative microbes, however, gram-positive microbes (with the exception of Corynebacterium glutamicum and Mycobacterium sp.), are typically resistant to the effects of levans. We demonstrate here (FIG. 50) that the sacB gene confers sucrose sensitivity of 2-3 logs in S. spinosa. Therefore our experiments indicate that sacB can be harnessed as counterselectable marker in S. spinosa for markerless strain generation. The sacB gene sequence was codon optimized for S. spinosa (SEQ ID No. 143).

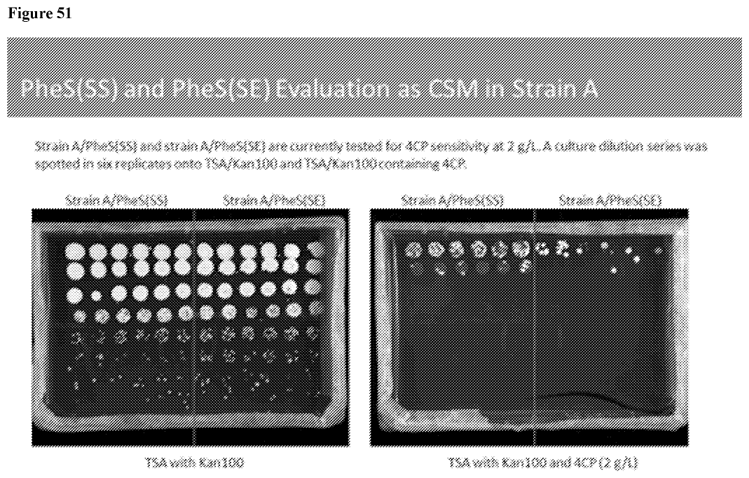

The pheS gene encodes the a subunit of phenylalanine-tRNA synthetase, which makes bacteria sensitive to 4-chlorophenylalanine (4CP) (Miyazaki, “Molecular engineering of a PheS counterselection marker for improved operating efficiency in Escherichia coli.” Biotechniques. 2015 Feb. 1; 58(2):86-8). In absence of 4-chlorophenylalanine, carriers of the pheS gene grow in a healthy manner, in presence of 4-chlorophenylalanine, however, only strains that have lost the pheS gene can survive. We demonstrate here (FIG. 51) that a mutated version of the pheS gene derived from Saccharopolyspora erythraea confers 4-cholorophenlylalanine sensitivity in S. spinosa and can hence be harnessed as counterselectable marker. pheS genes derived from both S. erythraea and S. spinosa with mutations described in Miyazaki 2015 were tested and were found functional (SEQ ID No. 144 and SEQ ID No. 145, respectively).

Vector backbones for strain engineering were designed in a number of configurations (FIGS. 49A-C) to alter strain engineering efficiency depending on background strain characteristics (e.g. basal strain resistance/sensitivity to selection and counteserlection agents). This includes using one or both counterselectable genes expressed with different promoters to alter the expression of the encoded markers.

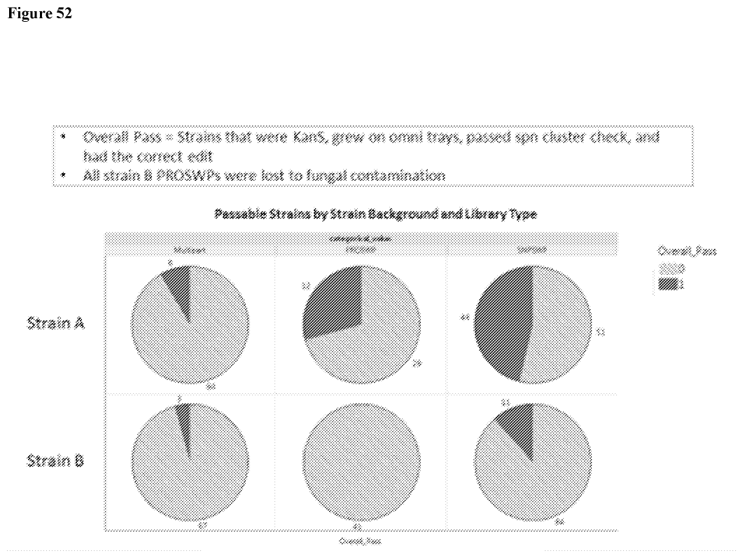

This tool was applied to HTP system of the present disclosure to generate engineered scarless S. spinosa strains, and the quality control results show successful application of the tool (FIG. 52). Thus, described here is the use of sacB and pheS as counterselection markers in S. spinosa and their application to gene editing. Microbial expression of counterselectable markers, or negative selection markers, causes restricted growth on a specific substrate (sucrose and 4-chlorophenylalanine respectively for sacB and pheS), and therefore enables the selection of microbes not containing the counterselection marker. sacB and pheS are described as counterselection markers in the literature in other hosts, but to our knowledge, this is the first characterization of their use in S. spinosa. Here, we use counterselection in combination with homologous recombination to perform targeted, scarless gene editing in S. spinosa, which is a powerful tool for HTP genomic engineering.

### Example 18: HTP Conjugation of Saccharopolyspora & Demonstration of Introducing Exogenous DNA into Saccharopolyspora

This example illustrates embodiments of the HTP genetic engineering methods of the present disclosure. Particularly, it demonstrates a high throughput process for interspecies conjugation of Saccharopolyspora (e.g., S. spinosa) using E. coli as a donor organism. This process enables the genetic modification of Saccharopolyspora (e.g., S. spinosa) using automation and automation-compatible cultivation formats to introduce genetic material by single crossover homologous recombination.

S. spinosa is an industrially relevant host and this invention enables highly parallelized efforts for genome engineering in this host to be realized. The results demonstrate that the methods of the present disclosure are able to generate rapid genetic changes of any kind or introduce any exogenous DNA, across the entire genome of a host cell.

Interspecies conjugation (a.k.a. intergeneric conjugation) is an effective mechanism for gene transfer in Saccharopolyspora, particularly to circumvent its potent restriction barrier. However, current methodologies for conjugation have yielded relatively low efficiencies and require manual procedures for completion (i.e. completed by a manual operator, with less than ten modifications at a time). The goal of this work was to improve conjugation efficiency in S. spinosa and to develop an automated protocol for conjugation to enable high-throughput (HTP) genome engineering in S. spinosa. Solving this problem necessitated 1) increasing conjugation efficiency to produce exconjugants in a HTP format and 2) developing automated protocols for culturing, plating, and colony picking.

We initiated protocol development for HTP conjugation using parameters from standard conjugation procedures on petri dishes. Several conjugation protocols using petri dishes were under development at the time this work began, and we selected an internal protocol as the basis for further development. Although the protocol resulted in lower conjugation efficiencies than other protocols, this protocol did not require any specialized steps that would require manual handling (e.g., cell scraping) and therefore was most amenable to automating.

We chose to take an integrated approach, working toward increasing conjugation efficiency while developing protocols for automated procedures in parallel. We initiated this process by using a Design of Experiment (DOE) approach to optimize the Early Strain protocol for conjugation on petri dishes, and this served as a basis for performing conjugation on 48-well divided Q-trays and additional DOE-based optimization. Compared with standard petri dishes, divided Q-trays maintain a 2D agar plate format with a reduced surface area (8-fold reduction from a petri dish) and interface well with automated systems. The 48-well Q-tray format provided a basis for development of standard procedures to automate the entire process of conjugation: donor cultivation, plating donor and recipient cells, antibiotic selection for exconjugants, exconjugant colony picking, patching and cultivation. The experimental inputs, including the design of experiment approach to explore the large parameter space of experimental factors for improved conjugation are provided below.

For initial experiments using DOE to improve conjugation efficiency, we chose to use the Definitive Screening Design strategy, which is generally effective for evaluating a large number of experimental factors in combination. Importantly, Definitive Screening Designs can identify the main effects governing a model in spite of factor interactions and they can also identify non-linear effects of quantitative factors. DOE is an optimization tool, and the limited experimental data for conjugation (i.e., experiments that resulted in non-zero exconjugants) suggested that multiple rounds of optimization would be required to achieve a protocol amenable to a HTP format.

Therefore, our work to improve conjugation efficiency took three general phases. In Phase I, we worked with experimental results that did not inform a statistically significant model of conjugation and improved efficiency through iteration. Upon identifying a set of conjugation conditions that repeatedly produced colonies, in Phase II, we attempted to identify new conditions that would further improve conjugation results. In Phase III, we used data on these conditions to develop a new set of experimental conditions for optimized conjugation, which were then validated with biological replicates across different operators.

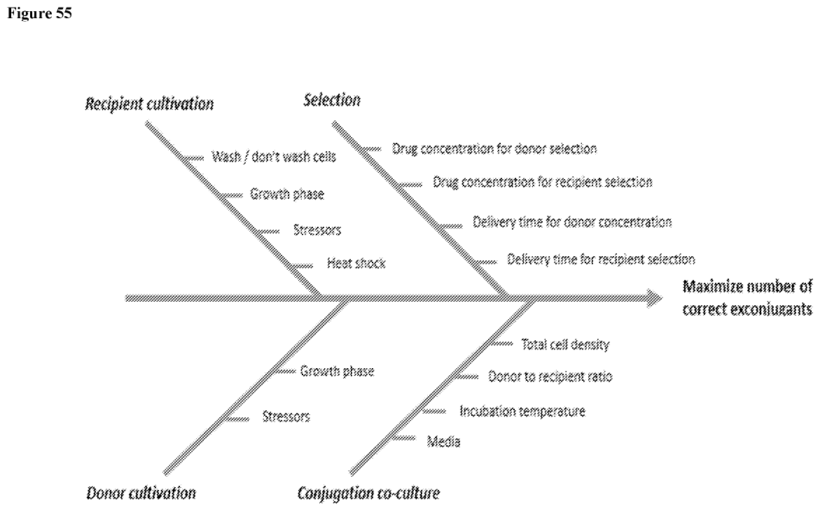

Factors considered for DOE-based optimization of conjugation were categorized into the four main parts of the conjugation protocol detailed in FIG. 55, which include cultivation of the recipient strain, cultivation of the donor strain, co-culture conjugation conditions, and selection of exconjugants. Each of these factors was considered for modification/optimization and was prioritized for experimental testing. Data was analyzed using JMP software version 11.2.1. Results are reported below in the context of statistical significance, unless otherwise stated.

The main steps for conjugation are:
1) Subculture recipient cells to mid-exponential phase
2) Subculture donor cells to mid-exponential phase
3) Combine donor and recipient cells
4) Plate donor and recipient cell mixture on conjugation media
5) Incubate plates to allow cells to conjugate
6) Apply antibiotic selection against donor cells
7) Apply antibiotic selection against non-integrated recipient cells
8) Further incubate plates to allow for the outgrowth of integrated recipient cells (exconjugants)

We describe the experiments and results for increasing conjugation efficiency in Section 1 below and development of automation procedures in Section 2.

### Section 1: Improving Conjugation Efficiency

### Experiment 1

### Experimental Goals:

To optimize conjugation on petri dishes using a DOE approach

### Experimental Design:

Conjugation on petri dishes using the Early Strain protocol resulted in low efficiencies and we anticipated that moving to a Q-tray format would result in even lower efficiencies due to the reduction in area. We therefore sought to improve conjugation efficiency on petri dishes such that the protocol transferred to a HTP format would have the greatest opportunity for success. To optimize the Early Strain protocol, we used a DOE approach and varied experimental conditions that were hypothesized to have the strongest influence on conjugation:
Recipient subculture time: 24-48 hrs
Nalidixic acid concentration: 14-50 ug/ml
Apramycin concentration: 36-100 ug/ml
Nalidixic acid delivery time: 2-24 hrs
Apramycin delivery time: 16-48 hrs
Expected donor concentration: 105-108
Expected recipient concentration: 105-109
Ratio of donor to recipient: 6:1, 1:100
Donor stress: no antibiotic stress or donor cells treated with 4 ug/ml-8 ug/ml nalidixic acid for 1.5 hrs

### Results:

Conditions that yielded exconjugants were shown in Table 13.

### Interpretation of Results:

(1) Condition 3 resulted in the greatest amount of exconjugants, yielding a total of 6 exconjugants per Q-tray well.
(2) Statistical analysis of experimental data did not show a significant effect of any single parameter on conjugation efficiency. However, of note, all conditions that produced colonies used donor and recipient antibiotic selection times separated by ≥24 hrs.

### Experiment 2

### Experimental Goals:

To determine if the Early Strain protocol for conjugation on petri dishes could be used for conjugation on divided Q-trays
To test if applying antibiotic stress to donor cells improves conjugation efficiency
To test if increased apramycin concentration for exconjugant positive selection improves conjugation efficiency

### Experimental Design:

1) We used two concentrations of recipient cells:
50 ul of a S. spinosa culture at OD=12, per the original petri dish protocol
5 ul of a S. spinosa culture at OD=12, considering the reduction in space of a Q-tray well
2) We used a fixed concentration of donor cells:
10-fold less than the original petri dish protocol, considering the reduction in space of a Q-tray well
3) We explored the effects of donor stress:
Half of the donor cell culture was treated with 4 ug/ml nalidixic acid for 1.5 hrs
Half of the donor cell culture remained untreated
4) We used two apramycin concentrations for selection of exconjugants:
Final concentration of 62.5 ug/ml agar
Final concentration of 100 ug/ml agar
5) Each condition was repeated across multiple wells to provide statistically significant data
   Results: Conditions that yielded exconjugants were shown in Table 14.

### Interpretation of Results:

1) Condition E resulted in the greatest amount of exconjugants, yielding a total of 1.5 exconjugants per Q-tray well.
2) Reduction in recipient cell concentration decreased conjugation efficiency.
3) Apramycin concentration and donor stress did not affect conjugation efficiency.
4) Overall, these results showed that conjugation could be performed on 48-well Q-trays.

### Experiment 3

### Experimental Goals:

To determine if optimized parameters for conjugation on petri dishes, from Experiment 1, could improve conjugation efficiency on Q-trays.

### Experimental Design:

We sought to test each set of conditions that yielded colonies on petri dishes for conjugation on Q-trays. However, because a Q-tray well is approximately 8-fold smaller in area than a petri dish, we were interested in testing two cell concentrations for conjugation on a single Q-tray well:
Approximately the same total cell concentrations used in the petri dish experiment.
Approximately ⅛ of the total cell concentrations used in the petri dish experiment.

### Results:

Conditions that yielded exconjugants were shown in Table 15.

### Interpretation of Results:

1) Condition #8 resulted in the greatest amount of exconjugants, yielding a total of 3.3 exconjugants per Q-tray well. Of note, this condition, scaled for donor concentration, had also resulted in the greatest amount of exconjugants in petri dish format.
2) Overall, our optimized petri dish conditions resulted in improved conjugation on 48-well Q-trays.

### Experiment 4

### Experimental Goals:

To run a DOE for conjugation on Q-trays to optimize conditions used for conjugation.

### Experimental Design:

from the results of above experiments, it was evident that conjugation could be performed on 48-well Q-trays. Because these efficiencies were very low, we sought to vary conditions that were anticipated to have the strongest influence on conjugation:
Recipient subculture time: 24-48 hrs
Nalidixic acid concentration: 25-100 ug/ml
Apramycin concentration: 50-200 ug/ml
Expected donor concentration: 10⁵-10⁶
Expected recipient concentration: 10⁵-10⁶
Ratio of donor to recipient: 3:1, 1:1, 1:3
Donor stress: no antibiotic stress or donor cells treated with 4 ug/ml nalidixic acid+4 ug/ml apramycin for 1.5 hrs

### Results:

Conditions that yielded exconjugants were shown in Table 16.

### Interpretation of Results:

The greatest number of exconjugants yielded was 0.7 exconjugants per Q-tray well.
This low value may have been attributed to the fact that Q-trays were incubated without being fully dry.
Performing an additional DOE would be important to understand if parameters tested were affected by inconsistent experimental conditions.

### Experiment 5

### Experimental Goals:

1) To run a DOE using condition #8 from Experiment 2 as a local optimum, varying experimental parameters around this condition.
2) To test if using the Tecan automated liquid handler for plating affects conjugation efficiency compared to manual plating (Note: up until this experiment, automated and manual liquid handling had both been used to complete conjugation, but it remained unclear if automated liquid handling resulted in greater or lesser conjugation efficiency compared to manual plating).
3) To test the effect of Q-tray dryness on conjugation.

### Experimental Design:

we sought to test each set of conditions that yielded colonies on petri dishes for conjugation on Q-trays. However, because a Q-tray well is approximately 8-fold smaller in area than a petri dish, we were interested in testing two cell concentrations for conjugation on a single Q-tray well:
1) Approximately the same total cell concentrations used in the petri dish experiment.
2) Approximately ⅛ of the total cell concentrations used in the petri dish experiment.

### Results:

Conditions that yielded exconjugants were shown in Table 17.

### Interpretation of Results:

Conditions 12 and 7 resulted in the greatest amount of exconjugants per Q-tray well, with condition 12 yielding a total of 8.4 exconjugants per Q-tray well.
Increasing apramycin concentration (200 ug/ml) resulted in increased conjugation efficiency.
Extra drying yielded a greater total number of exconjugants, although these data were not statistically significant. Furthermore, extra drying resulted in plates becoming cracked and too thin which was challenging for downstream procedures, such as colony picking.
Automated liquid handling did not affect conjugation efficiency compared to manual plating.
At this point in our experimental plan, we had identified multiple conditions that yielded ≥5 colonies per Q-tray well. Although we did not have data to construct a statistically significant linear model for conjugation, these conditions suggested that we had pinpointed certain experimental conditions that could be further improved by exploring new factors.

### Experiment 6

### Experimental Goals:

1) To identify new experimental factors to further improve conjugation efficiency and inform a statistical model for conjugation
2) To run a DOE around Q-tray media components to determine optimal media conditions for conjugation

### Experimental Design:

We chose the following conditions to vary:
ISP4 powder: 27.8 g/L-55.5 g/L
Yeast extract: 0.5 g/L-2 g/L
Glucose: 1.5 g/L-6 g/L
MgCl2: 10 mM-40 mM
Additional agar: 0 g/L-7.5 g/L
   We chose to test the effects of these different media conditions using experimental conditions that reflected previous high-performing conditions and additional new conditions:
condition #12 from Experiment 5;
condition #8: using higher nalidixic acid and apramycin concentrations to facilitate plating procedures;
altered version of condition #8, termed #8A, to account for donor cell concentration variability;
Four new conditions were generated by varying donor to recipient ratios between 15:1 to 1:5 and the total expected cell concentration between 105-106 based on previous results.

### Results:

Conditions that yielded exconjugants were shown in Table 18.

### Interpretation of Results:

High glucose resulted in increased conjugation efficiency. All other media components were not determined to have a significant effect on conjugation efficiency.
High nalidixic concentration (100 ug/ml) resulted in increased conjugation efficiency.
Non-linear partition modeling by JMP predicted that lower apramycin concentration (100 ug/ml) would increase conjugation efficiency.
Conditions #12, #8A, and #8, in order, resulted in the greatest numbers of exconjugants, with condition #12 yielding 18 exconjugants/Q-tray well.

### Experiment 7

### Experimental Goals:

To re-run top performing conditions and test if varying donor and recipient concentrations could improve performance of these conditions

### Experimental Design:

1) We chose condition #7 from Experiment 5, condition #12 from Experiment 5, and condition #8 from Experiment 6 as baseline conditions.
2) We chose to alter donor and recipient concentrations from these baseline conditions using quantified variability across experiments. Because our protocol uses OD as a proxy for cell concentration, there is inherent variability in donor and recipient concentrations between experiments. We calculated this amount of variation (CV), and altered donor and recipient concentrations proportionally. We performed conjugation experiments with all combinations of low (proportional decrease by CV), high (proportional increase by CV), and baseline donor and recipient concentrations.

### Results:

Conditions that yielded exconjugants were shown in Table 19.

### Interpretation of Results:

1) Low or high donor and recipient concentrations did not improve conjugation efficiency.
2) Condition #8 and condition #12 with original baseline cell concentrations resulted in the highest numbers of exconjugants, with ˜5 exconjugants per Q-tray well.

### Experiment 8

### Experimental Goals:

1) To repeat conditions #8A and #12 from media optimization experiment on experiment 6 to validate that new media conditions improved conjugation efficiency.
2) To test if condition #12 adjusted to reflect JMP predictions from experiment 6 (apramycin concentration 100 ug/ml) improved conjugation efficiency (this condition was termed #12JA)
3) To test condition #7 from Experiment 5 using new media conditions, since condition 7 had been demonstrated to perform well on standard media

### Experimental Design:—

We ran conditions #12, #12JA, #8A, and #7 on new media conditions and on the standard conjugation media conditions for comparison.

### Results:

Conditions that yielded exconjugants were shown in Table 20.
Interpretation of results: The highest number of exconjugants for this experimental design was for condition #12JA, yielding 40 exconjugants/Q-tray well.
New media conditions were validated to improve conjugation efficiency.
Lower apramycin concentration resulted in a greater number of exconjugants/Q-tray well, although there were not enough data to assess statistical significance.

### Experiment 9

### Experimental Goals:

To evaluate sensitivities around current optimized conjugation conditions by using donor and recipient cells at incorrect densities/growth state. This would provide an indication of how sensitive the conjugation protocol is to concentration or growth phase of cells, as variability of these parameters would be expected to occur from site to site.

### Experimental Design:

We used conditions #8 and #12 as baseline conditions for conjugation experiments.
We performed conjugation experiments of all combinations of low, standard, and high densities for donor and recipient cells.
Low donor cell cultures were used at OD600=0.2
Standard donor cell cultures were used at OD600=0.4
High donor cell cultures were used at OD600=0.8
Low recipient cell cultures at OD540=9.6
Standard recipient cell cultures at OD540=13.0
High recipient cell cultures at OD540=14
Experiments were performed using new optimized media conditions (media 3 from experiment on Experiment 6).

### Results:

1) Using donor cells at low density resulted in a ˜60% reduction in total exconjugants.
2) Using donor cells at high density resulted in a ˜50% reduction in total exconjugants.
3) Using recipient cells at low density resulted in ˜80% reduction in total exconjugants.
4) Using recipient cells at high density resulted in 0 total exconjugants.
Interpretation of results: Condition #12 with standard cell densities resulted in 40 exconjugants/Q-tray well.
Incorrect donor and recipient cell concentrations/growth phases resulted in much lower conjugation efficiencies, with correct recipient culture conditions being particularly important.

### Experiment 10

### Experimental Goals:

1) To validate optimized conditions in the hands of a new operator
2) To evaluate sensitivities around current optimized conjugation conditions by using donor and recipient cells at incorrect densities/growth state.

### Experimental Design:

We used condition #12JA with new optimized media conditions.
We performed conjugation experiments of all combinations of low, standard, and high densities for donor and recipient cells.
Low donor cell cultures were used at OD600=0.3
Standard donor cell cultures were used at OD600=0.4
High donor cell cultures were used at OD600=1.0
Low recipient cell cultures at OD540=4.6
Standard recipient cell cultures at OD540=8.0
High recipient cell cultures at OD540=10.6

### Results:

Using donor cells at low density resulted in a ˜100% increase in total exconjugants.
Using donor cells at high density resulted in a ˜70% increase in total exconjugants.
Using recipient cells at low density resulted in ˜80% reduction in total exconjugants.
Using recipient cells at high density resulted in ˜80% reduction in total exconjugants.

### Interpretation of Results:

1) Condition #12JA completed by a new operator resulted in 15 exconjugants/Q-tray well. This was a reduction from previous results, and was likely due to the new operator attempting the procedure for the first time.
2) Sensitivity of recipient cell concentration/growth phase was consistent with experimental results determined by previous operator on in experiment 9.
3) Results using incorrect donor cell concentration/growth phase were inconsistent with data from in Experiment 9. Using incorrect donor cell concentrations resulted in improved conjugation efficiency from standard protocol, however these data were of inconclusive significance in the context of previous experimental data.
4) Microscopy of recipient cells was useful in verifying cell state. Late log cells appear more fragmented in liquid culture.

TABLE 13
Results from Design of Experiment based optimization of low-throughput conjugation

| | | | | | | | | | NA con- | Apra | |
|---|---|---|---|---|---|---|---|---|---|---|---|
| | Recipient | Con- | Recipient | | | | NA | Apra | centration | concentration | |
| | wash | jugation | subculture | Donor | Ratio | | delivery | delivery | (ug/ml | (ug/ml | #exconjugants/ |
| | condition | temp (C.) | time (hr) | stres | (D:R) | Total cells | time | time | agar) | agar) | petridish |
| 1 | no wash | 30 | 24 | 8 | 0.10 | 2.74E+06 | 16 | 46 | 28 | 36 | 4 |
| 2 | no wash | 37 | 36 | 4 | 1.00 | 3.34E+07 | 16 | 41.5 | 28 | 54 | 3 |
| 3 | wash | 30 | 48 | 0 | 42.2 | 5.00E+06 | 21 | 48 | 28 | 72 | 6 |
| 4 | no wash | 37 | 24 | 0 | 1.53 | 1.00E+07 | 24 | 48 | 14 | 100 | 2 |
| 5 | no wash | 30 | 24 | 4 | 2.45 | 8.40E+06 | 16 | 42 | 32 | 68 | 3 |
| SOP | no wash | 30 | 48 | 0 | NA | NA | 20 | 20 | 14 | 35 | 0 |

TABLE 14
Results from initial Q-tray conjugation experiment 2

| | | Donor Stress | | |
|---|---|---|---|---|
| | | Nalidixic | Apramycin | # Exconjugants/ |
| | Recipient | acid | concentration | Q-tray |
| Condition | (cfu/ml) | (ug/ml) | (ug/ml agar) | well |
| A | 1.65 × 10⁵ | 0 | 62.5 | 0.5 |
| B | 1.65 × 10⁵ | 4 | 62.5 | 0.5 |
| C | 1.65 × 10⁴ | 0 | 62.5 | 0 |
| D | 1.65 × 10⁴ | 4 | 62.5 | 0 |
| E | 1.65 × 10⁵ | 0 | 100 | 1.5 |
| F | 1.65 × 10⁵ | 4 | 100 | 0.5 |
| G | 1.65 × 10⁴ | 0 | 100 | 0 |
| H | 1.65 × 10⁴ | 4 | 100 | 0 |

TABLE 15
Results from transferring LTP conjugation conditions to HTP format

| | | | | | | | Relative | | | NA | Apra | |
|---|---|---|---|---|---|---|---|---|---|---|---|---|
| | | | | Donor | | | amount of | | Apra | concen- | concen- | |
| | Recipient | - | Recipient | stress | | Total | cell from | NA | delivery | tration | tration | #exconjugants/ |
| | wash | Conjugation | subculture | NA | Ratio | cells | LTP | delivery | time | (ug/ml | (ug/ml | Q- |
| | condition | temp | time (hrs) | (ug/ml) | (D:R) | (cfu/ml) | condition | time (hrs) | (hrs) | agar) | agar) | tray well |
| 1 | no wash | 30 | 24 | 8 | 0.04 | 2.1E+06 | 1x | 16 | 46 | 28 | 36 | 0.2 |
| 2 | no wash | 37 | 36 | 4 | 1.15 | 7.3E+06 | 1x | 16 | 41.5 | 28 | 54 | 0.0 |
| 3 | wash | 30 | 48 | 0 | 1.43 | 2.1E+06 | 1x | 21 | 48 | 28 | 72 | 1.5 |
| 4 | no wash | 37 | 24 | 0 | 0.06 | 3.6E+07 | 1x | 24 | 48 | 14 | 100 | 0.0 |
| 5 | no wash | 30 | 24 | 4 | 0.29 | 2.7E+07 | 1x | 16 | 42 | 32 | 68 | 0.5 |
| SOP | no wash | 30 | 48 | 0 | 16.67 | 2.4E+06 | 1x | 20 | 20 | 14 | 35 | 0.2 |
| 6 | no wash | 30 | 24 | 8 | 0.04 | 3.5E+05 | 1/8x | 16 | 46 | 28 | 36 | 0.0 |
| 7 | no wash | 37 | 36 | 4 | 1.15 | 1.2E+06 | 1/8x | 16 | 41.5 | 28 | 54 | 0.0 |
| 8 | wash | 30 | 48 | 0 | 1.43 | 3.5E+05 | 1/8x | 21 | 48 | 28 | 72 | 3.3 |
| 9 | no wash | 37 | 24 | 0 | 0.06 | 6.0E+06 | 1/8x | 24 | 48 | 14 | 100 | 0.0 |
| 10 | no wash | 30 | 24 | 4 | 0.29 | 4.4E+06 | 1/8x | 16 | 42 | 32 | 68 | 0.7 |
| SOP | no wash | 30 | 48 | 0 | 16.67 | 4.0E+05 | 1/8x | 20 | 20 | 14 | 35 | 0.0 |
| (dilute ) | | | | | | | | | | | | |

TABLE 16
Best conditions from conjugation experiment 4

| | | | NA | | NA | Apra | | | |
|---|---|---|---|---|---|---|---|---|---|
| Recipient | | Recipient | delivery | Apra | concentration | concentration | | Total cell | |
| wash | Conjugation | subculture | time | delivery | (ug/ml | (ug/ml | Ratio | concentration | #exconjugants/ |
| condition | temp (C.) | time (hrs) | (hrs) | time (hrs) | agar) | agar) | (D:R) | (cfu/ml) | well |
| Wash | 30 | 48 | 21 | 45 | 62.5 | 50 | 1.1 | 1.5E+0.6 | 0.7 |

TABLE 17
Best conditions from conjugation experiment 5

| | | | | NA | Apra | | Apra | | | |
|---|---|---|---|---|---|---|---|---|---|---|
| | Recipient | | Recipient | delivery | delivery | NA | concentration | | Total cell | |
| | wash | Conjugation | subculture | time | time | concentration | (ug/ml | Ratio | concentration | #exconjugants/ |
| | condition | temp (C) | time (hrs) | (hrs) | (hrs) | (ug/ml agar) | agar) | (D:R) | (cfu/ml) | well |
| 12 | Wash | 30 | 48 | 20 | 42 | 100 | 200 | 14.3 | 3.0E+0.6 | 8.4 |
| 7 | Wash | 30 | 24 | 20 | 42 | 62.5 | 200 | 0.1 | 1.5E+0.6 | 5.4 |

TABLE 18
Best conditions from conjugation experiment 6

| | | | | | NA | | | Apra | | | |
|---|---|---|---|---|---|---|---|---|---|---|---|
| | | Recipient | | Recipient | delivery | Apra | NA | concentration | | Total cell | |
| | | wash | Conjugation | subculture | time | delivery | concentration | (ug/ml | Ratio | concentration | #exconjugants/ |
| | Media | condition | temp (C) | time (hrs) | (hrs) | time (hrs) | (ug/ml agar) | agar) | (D:R) | (cfu/ml) | well |
| 12 | 3 | Wash | 30 | 48 | 18 | 42 | 100 | 200 | 8.1 | 1.76E+06 | 18.0 |
| 8A | 3 | Wash | 30 | 48 | 18 | 42 | 50 | 100 | 0.8 | 2.66E+05 | 7.5 |
| 8 | 8 | Wash | 30 | 48 | 18 | 42 | 50 | 100 | 5.5 | 9.47E+05 | 6.5 |

TABLE 19
Best conditions from conjugation experiment 7

| | | | | | NA | Apra | NA | Apra | | | |
|---|---|---|---|---|---|---|---|---|---|---|---|
| | | Recipient | | Recipient | delivery | delivery | concentration | concentration | | Total cell | |
| | | wash | Conjugation | subculture | time | time | (ug/ml | (ug/ml | | concentration | #exconjugants/ |
| | Media | condition | temp (° C.) | time (hrs) | (hrs) | (hrs) | agar) | agar) | Ratio | (cfu/ml) | well |
| 12 | Standard | Wash | 30 | 48 | 20 | 42 | 100 | 200 | 0.006 | 3.87E+08 | 5.2 |
| 8 | Standard | Wash | 30 | 48 | 20 | 42 | 50 | 100 | 0.019 | 6.54E+07 | 5.5 |

TABLE 20
Best conditions from conjugation experiment 8

| | | | | | NA | | | Apra | | | |
|---|---|---|---|---|---|---|---|---|---|---|---|
| | | Recipient | | Recipient | delivery | Apra | NA | concentration | | Total cell | |
| | | wash | Conjugation | subculture | time | delivery | concentration | (ug/ml | Ratio | concentration | #exconjugants/ |
| | Media | condition | temp (C) | time (hrs) | (hrs) | time (hrs) | (ug/ml agar) | agar) | (D:R) | (cfu/ml) | well |
| 12J | 3 | Wash | 30 | 48 | 20 | 42 | 100 | 100 | 1.23 | 4.19E+06 | 39.9 |
| 12 | 3 | Wash | 30 | 48 | 20 | 42 | 100 | 200 | 1.23 | 4.19E+06 | 19.9 |
| 8A | 3 | Wash | 30 | 48 | 20 | 42 | 50 | 100 | 0.19 | 1.11E+06 | 0.8 |
| 7 | 3 | Wash | 30 | 24 | 20 | 42 | 62.5 | 200 | 0.08 | 2.02E+06 | 1.1 |

### Section 2: Automation Development

### Experiment 11. High Throughput Donor Cultivation (Automation Component)

### Experimental Goals:

To grow donor cells in a HTP format for conjugation

### Experimental Design:

1) We tested growth of E. coli donor cultures in 96 well deep well square plates (E&K EK-2440-ST). Cultures were inoculated by normalizing inoculation volume based on OD600 of overnight culture such that the culture with the lowest OD reading corresponded to a 1:100 inoculation.
2) We tested three volumes of LB media for growth: 250 ul, 500 ul, 750 ul.
3) To assess the effects of HTP growth on conjugation, we performed conjugation using E. coli S17+SS015 grown in this HTP format.

### Results:

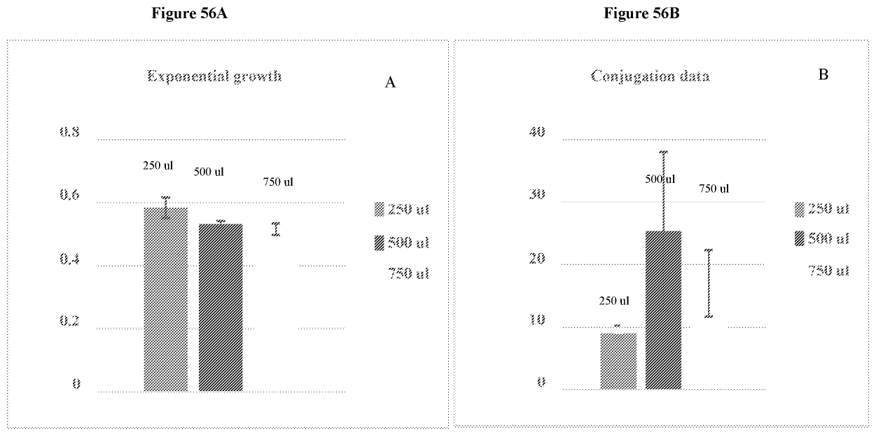

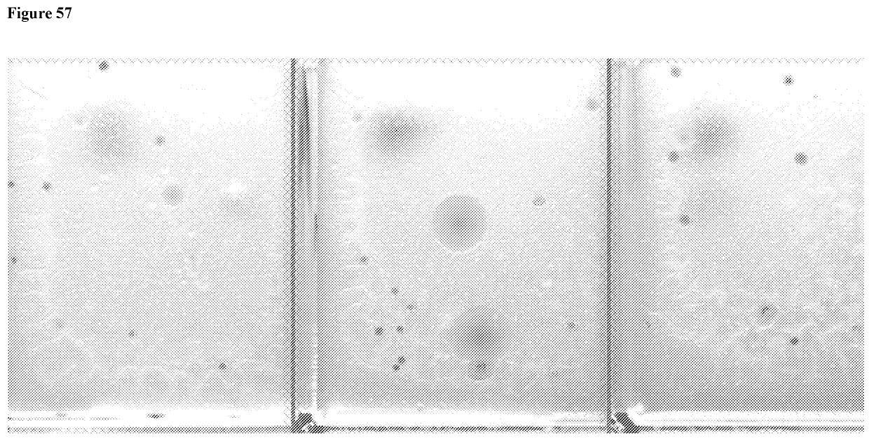

Cell growth and conjugation data is shown in FIG. 56A-B.

### Interpretation of Results:

Cultures grew robustly at all volumes tested. In addition, cultures grown at a volume of 500 ul yielded the highest number of exconjugants, although differences were not statistically significant. The 500 ul volume offered easy liquid handling and ample volume for checking ODs and was therefore selected for high throughput donor growth.

### Experiment 12. Plating Cells and Antibiotics in an HTP Format for Conjugation (Automation Component)

### Experimental Goals:

1) To plate cells and antibiotics in a HTP format
2) To achieve consistent plating throughout the conjugation protocol, since multiple plating steps to layer antibiotics on top of donor and recipient cells are required for conjugation

### Experimental Design:

We identified three potential procedures for plating cells and antibiotics on 48-well divided Q-trays:
Spot plating—plating liquid volume in a single spot and letting it dry in the area it was plated
Plating with beads—plating liquid volume in a single spot, then using beads to disperse the liquid over the whole area of the well
Flooding a Q-tray well—plating ample liquid volume such that with a rocking motion, liquid would be dispersed over the whole area of the well

### Results:

Spot plating resulted in inconsistent cell plating and additionally, the hydrophobicity of plated cells made it challenging to plate antibiotics for exconjugant selection using this method. The spotted antibiotic volume did not disperse to the full area of the plated cells and could not be spread without manually breaking the surface tension.
Plating with beads resulted in consistent plating but at the consequence of contamination. Shaking Q-trays containing beads in each well caused splattering of plated liquid, and on occasion beads would cross between wells. Additionally, plating with beads would require significant customization to interface with an automated system.
Flooding a Q-tray well allowed for consistent plating throughout the conjugation procedure, such that cells and antibiotics were plated evenly over the well area. However, plates required long incubation periods to dry completely after cultures and antibiotics were plated.
Additionally, an automated solution would need to be developed to rock plates back and forth to disperse liquid.

### Interpretation of Results:

Based on our plating trials, we found that plating enough liquid to flood a Q-tray well was the most promising procedure to use for automated conjugation. This procedure resulted in consistent, even plating and could readily interface with an automated liquid handler.
To overcome the manual step of rocking Q-trays to disperse liquid, we purchased the 3D Rotator Wave from VWR and modified its platform with a custom part to accommodate Q-tray dimensions. Because the 3D Rotator Wave can move in orbital motion and also in the z plane, it provided the same movement as when plate rocking was performed manually.

### Experiment 13. Exconjugant Picking (Automation Component)

### Experimental Goals:

To develop a standard procedure for detecting exconjugants on conjugation plates
To patch/stamp colonies from conjugation Q-trays onto selective agar omni trays

### Experimental Design:

1) We used the Qpix 420 and corresponding software to identify S. spinosa exconjugants on conjugation plates.
2) We experimented with the following imaging parameters to detect exconjugants:
   Threshold
   Exposure
   Gain
   Inverting image
   Subtract background
3) We experimented with the following feature selection parameters to include detect pickable exconjugants:
   1) Compactness
   2) Axis ratio
   3) Min diameter
   4) Max diameter
   5) Min proximity
4) We tested using the picking head with two different types of pins:
   1. Yeast picking pins (X4377)
   2. E. coli picking head (X4370)
5) We tried using two different functions for inoculation onto solid agar omni trays in an effort to produce a large, robust patch:
   Single dip
   Stir

### Results:

We found that inverting the image during the Q-tray imaging process was very useful for detecting S. spinosa exconjugants.
After imaging multiple conjugation plates, we found that no single threshold and exposure values could be used to accurately identify S. spinosa exconjugants (See FIG. 57). Because of the variability of background on each plate (e.g. leftover dead donor and recipient cells) it was necessary to adjust threshold and exposure values for each plate. We identified a range of values to use for these parameters.
We found that using E. coli pins to transfer exconjugants did not work—this was likely because these pins cannot pick S. spinosa cells well enough to allow for subsequent inoculation.
Colony picking with yeast pins worked well for plate inoculation. However, after picking, the picking head did not fully detach from the omni tray and carried the omni tray along with it. After taping the destination omni tray down, this was no longer an issue.
The dip function worked well for inoculation. The stir function also seemed like a promising method for inoculation. Unfortunately these results were not conclusive as omni trays inoculated with the stir function experienced fungal contamination.

### Interpretation of Results:

We established a general set of parameters for picking S. spinosa exconjugants that could be adjusted based on plate variability using the Qpix picking head fitted with yeast pins and the dip inoculation function. This protocol resulted in robust S. spinosa growth with no visible E. coli contamination.

### Experiment 14

### Experimental Goals:

To pick exconjugant patches from omni trays into 96 well deep well plates for cultivation and stocking.

### Experimental Design:

1. We tested picking of S. spinosa patches into in 96 well deep well square plates (E&K EK-2440-ST) using standard picking conditions.
2. We tested three volumes of DAS media 2 for growth: 300 ul, 400 ul, 500 ul.

### Results:

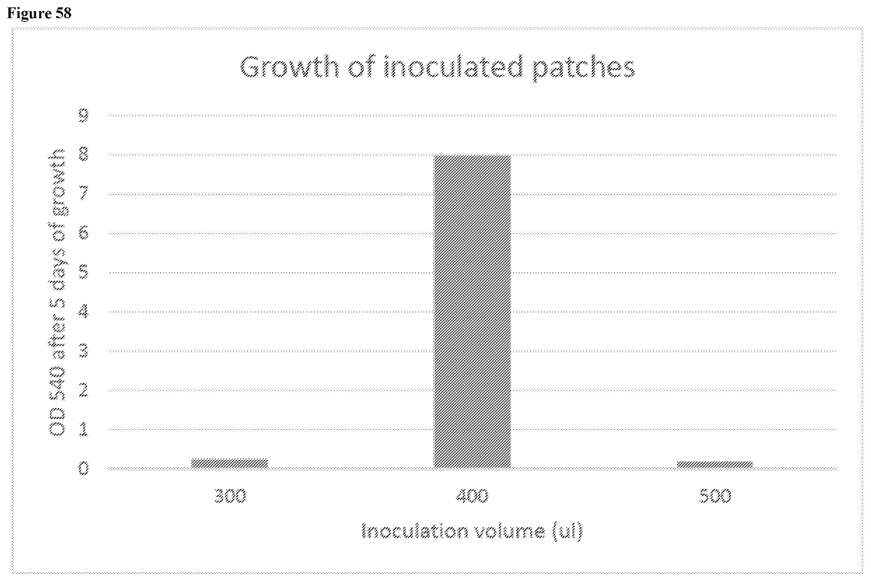

As shown in FIG. 58, only wells inoculated using 400 ul of media resulted in robust growth.

### Interpretation of Results:

1. It was unclear as to why 300 ul and 500 ul inoculation volumes resulted in no growth of exconjugant cultures. We suspected that this was associated with the inoculation process rather than the media volume itself.
2. This protocol will require some additional validation and optimization to ensure robust growth of picked exconjugant patches.

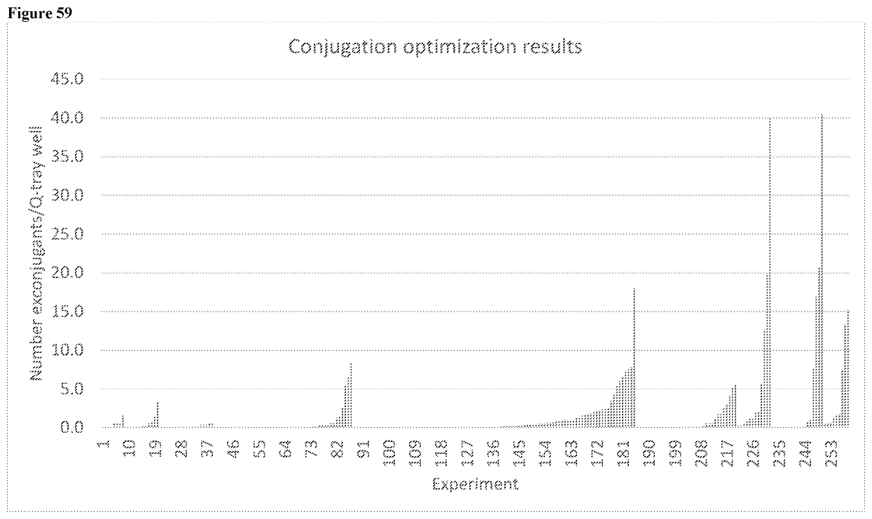

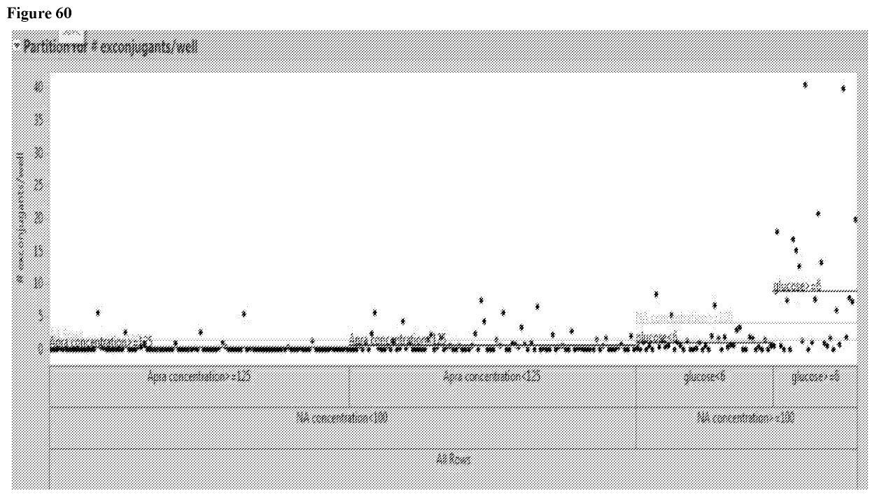

Summary: FIG. 59 summarizes results of conjugation experiments completed through course of DOE-based optimization. From the optimization process, statistical analyses suggested that the most critical conditions for conjugation were drug selection concentrations and glucose concentration of the media (See FIG. 60). Experimental analyses further suggested that the recipient culture growth stage was also a critical condition for conjugation. Optical density readings appear to be a relatively good indicator of determining when the recipient culture is ready for conjugation, however more recent experiments suggest that cell morphology is also useful in verifying cell state. Therefore recipient cultures should be checked for correct cell morphology in addition to optical density. The optimized conjugation protocol did not show great sensitivity around donor concentration and growth phase, and therefore the established protocol may not be sensitive to deviations in donor cell growth. This will be convenient when working with various strains in a HTP format.

This invention enables a process for improved conjugation efficiency in Saccharopolyspora (e.g., S. spinosa), accompanying an automated protocol for conjugation to enable high-throughput (HTP) genome engineering. We developed a protocol for high-throughput conjugation that resulted in an overall average of 24 exconjugants/Q-tray well (run in duplicate by two separate operators). Conjugation conditions yielding the maximum number of exconjugants included: washing recipient cells, conjugating at 30° C., subculturing the recipient strain for approximately 48 hrs, selecting with 100 ug/ml nalidixic acid 20 hrs after conjugation, selecting with 100 ug/ml apramycin 42 hrs after conjugation, using ISP4 modified media with 6 g/L glucose, donor to recipient ratio of ˜1:0.8, with a total number of ˜7×10⁶ cells.

Sequences of the Disclosure with SEQ ID NO Identifiers

| | | | |
|---|---|---|---|
| | | | SEQ |
| | | | ID |
| Source | Name | Description | NO: |

Promoter Ladder

| | | | |
|---|---|---|---|
| Saccharopolyspora | P7160 | Promoter sequence associated with chaperonin | 1 |
| spinosa | | GroEL | |
| Saccharopolyspora | P7253 | Promoter sequence associated with Elongation factor | 2 |
| spinosa | | Tu | |
| Saccharopolyspora | P6681 | Promoter sequence associated with F-type ATPase | 3 |
| spinosa | | subunit delta | |
| Saccharopolyspora | P6316 | Promoter sequence associated with PspA/IM30 | 4 |
| spinosa | | family protein | |
| Saccharopolyspora | P6806 | Promoter sequence associated with 2-oxoglutarate | 5 |
| spinosa | | decarboxylase | |
| Saccharopolyspora | P3159 | Promoter sequence associated with putative enoyl- | 6 |
| spinosa | | CoA hydratase echA8 | |
| Saccharopolyspora | P0757 | Promoter sequence associated with putative L- | 7 |
| spinosa | | lysine-epsilon aminotransferase | |
| Saccharopolyspora | P5011 | Promoter sequence associated with hypothetical | 8 |
| spinosa | | protein | |
| Saccharopolyspora | P1409 | Promoter sequence associated with NAD-specific | 9 |
| spinosa | | glutamate dehydrogenase | |
| Saccharopolyspora | P4735 | Promoter sequence associated with leucyl | 10 |
| spinosa | | aminopeptidase (aminopeptidase T) | |
| Saccharopolyspora | P2900 | Promoter sequence associated with Cytochrome | 11 |
| spinosa | | P450-terp | |
| Saccharopolyspora | P0801 | Promoter sequence associated with Periplasmic | 12 |
| spinosa | | murein peptide-binding protein precursor | |
| Synthetic | P21 | Synthetic promoter described in Siegl et al. | 13 |
| Synthetic | PA9 | Synthetic promoter described in Siegl et al. | 14 |
| Synthetic | PA3 | Synthetic promoter described in Siegl et al. | 15 |
| Synthetic | PB4 | Synthetic promoter described in Siegl et al. | 16 |
| Synthetic | PB12 | Synthetic promoter described in Siegl et al. | 17 |
| Synthetic | PB1 | Synthetic promoter described in Siegl et al. | 18 |
| Synthetic | PC1 | Synthetic promoter described in Siegl et al. | 19 |
| Synthetic | P72 | Synthetic promoter described in Siegl et al. | 20 |
| Synthetic | P-C4-1 | Synthetic promoter described in Seghezzi et al. | 21 |
| Synthetic | P-A5-19 | Synthetic promoter described in Seghezzi et al. | 22 |
| Synthetic | P-C4-14 | Synthetic promoter described in Seghezzi et al. | 23 |
| Synthetic | P-D1-7 | Synthetic promoter described in Seghezzi et al. | 24 |
| Saccharopolyspora | P1 | Promoter sequence associated secreted protein | 25 |
| spinosa | | | |
| Saccharopolyspora | P2 | Promoter sequence associated hypothetical protein | 26 |
| spinosa | | | |
| Saccharopolyspora | P3 | Promoter sequence associated RNA polymerase | 27 |
| spinosa | | sigma factor SigD | |
| Saccharopolyspora | P3v2 | Promoter sequence associated RNA polymerase | 28 |
| spinosa | | sigma factor SigD, version 2 | |
| Saccharopolyspora | P4 | Promoter sequence associated Antigen Ag88 | 29 |
| spinosa | | | |
| Saccharopolyspora | P4v2 | Promoter sequence associated Antigen Ag88, | 30 |
| spinosa | | version 2 | |
| Saccharopolyspora | P5 | Promoter sequence associated DNA-directed RNA | 31 |
| spinosa | | polymerase subunit beta | |
| Saccharopolyspora | P5v2 | Promoter sequence associated DNA-directed RNA | 32 |
| spinosa | | polymerase subunit beta | |
| Saccharopolyspora | P6 | Promoter sequence associated molecular chaperone | 33 |
| spinosa | | GroEL | |
| Saccharopolyspora | P7 | Promoter sequence associated UDP-4-amino-4- | 34 |
| spinosa | | deoxy-L-arabinose--oxoglutarate aminotransferase | |
| Saccharopolyspora | P8 | Promoter sequence associated Proline racemase | 35 |
| spinosa | | | |
| Saccharopolyspora | P9 | Promoter sequence associated Phenyloxazoline | 36 |
| spinosa | | synthase MbtB | |
| Saccharopolyspora | PspnA | Promoter sequence associated polyketide synthase | 37 |
| spinosa | | loading and extender module 1 | |
| Saccharopolyspora | PspnA_v2 | Promoter sequence associated polyketide synthase | 38 |
| spinosa | | loading and extender module 1, version 2 | |
| Saccharopolyspora | PspnF | Promoter sequence associated methyltransferase-like | 39 |
| spinosa | | protein | |
| Saccharopolyspora | PspnG | Promoter sequence associated putative NDP- | 40 |
| spinosa | | rhamnosyltransferase | |
| Saccharopolyspora | PspnQ_v2 | Promoter sequence associated putative NDP-hexose-3 | 41 |
| spinosa | | | |
| Saccharopolyspora | PspnQ_v2 | Promoter sequence associated putative NDP-hexose- | 42 |
| spinosa | | 3, version 2 | |
| Synthetic | P21_mutant | Synthetic promoter | 43 |
| Saccharopolyspora | P1_core | Promoter sequence associated secreted protein | 44 |
| spinosa | | | |
| Saccharopolyspora | P1(−33) | Promoter sequence associated secreted protein | 45 |
| spinosa | | | |
| Saccharopolyspora | P1 + ribswtch | Promoter sequence associated secreted protein | 46 |
| spinosa | | | |
| Synthetic | P21-P1 | Synthetic promoter | 47 |
| Synthetic | P1-P21 | Synthetic promoter | 48 |
| Saccharopolyspora | P1765 | Promoter sequence associated Glutamine synthetase 1 | 49 |
| spinosa | | | |
| Saccharopolyspora | P3747 | Promoter sequence associated hypothetical protein | 50 |
| spinosa | | | |
| Saccharopolyspora | P5078 | Promoter sequence associated hypothetical protein | 51 |
| spinosa | | | |
| Saccharopolyspora | P7419 | Promoter sequence associated anaerobic benzoate | 52 |
| spinosa | | catabolism transcriptional regulator | |
| Saccharopolyspora | P7156 | Promoter sequence associated RNA polymerase | 53 |
| spinosa | | sigma factor SigD | |
| Saccharopolyspora | P7256 | Promoter sequence associated 30S ribosomal | 54 |
| spinosa | | protein S12 | |
| Saccharopolyspora | P1941 | Promoter sequence associated Response regulator | 55 |
| spinosa | | protein vraR | |
| Saccharopolyspora | P3405 (P8) | Promoter sequence associated Proline racemase | 56 |
| spinosa | | | |
| Saccharopolyspora | P3407 | Promoter sequence associated ABC transporter | 57 |
| spinosa | | arginine-binding protein 1 precursor | |
| Saccharopolyspora | P2428 | Promoter sequence associated Promoter sequence | 58 |
| spinosa | | associated acetyl-CoA synthetase | |
| Saccharopolyspora | P0927 | Promoter sequence associated 4- | 59 |
| spinosa | | hydroxyphenylpyruvate dioxygenase | |
| Saccharopolyspora | P0889 | Promoter sequence associated Linear gramicidin | 60 |
| spinosa | | dehydrogenase LgrE | |
| Saccharopolyspora | P0186 | Promoter sequence associated L,D-transpeptidase | 61 |
| spinosa | | catalytic domain | |
| Saccharopolyspora | P3702_v2 | Promoter sequence associated hypothetical protein | 62 |
| spinosa | | | |
| Saccharopolyspora | P7156_v2 | Promoter sequence associated RNA polymerase | 63 |
| spinosa | | sigma factor SigD | |
| Saccharopolyspora | P7256_v2 | Promoter sequence associated 30S ribosomal | 64 |
| spinosa | | protein S12 | |
| Saccharopolyspora | P1765_v2 | Promoter sequence associated Glutamine synthetase 1 | 65 |
| spinosa | | | |
| Saccharopolyspora | P7539_v2 | Promoter sequence associated Antigen Ag88 | 66 |
| spinosa | | | |
| Saccharopolyspora | P7276_v2 | Promoter sequence associated DNA-directed RNA | 67 |
| spinosa | | polymerase subunit beta | |
| Saccharopolyspora | P0941_v2 | Promoter sequence associated hypothetical protein | 68 |
| spinosa | | | |
| Saccharopolyspora | P0889_v2 | Promoter sequence associated Linear gramicidin | 69 |
| spinosa | | dehydrogenase LgrE | |
| Synthetic | Pmut-1 | Synthetic promoter | 172 |
| Synthetic | B2 | Synthetic promoter | 173 |
| Synthetic | D1 | Synthetic promoter | 174 |
| Synthetic | D2 | Synthetic promoter | 175 |

putative terminators

| | | | |
|---|---|---|---|
| Saccharopolyspora | T1 | Terminator sequence associated with elongation | 70 |
| spinosa | | factor tu | |
| Saccharopolyspora | T2 | Terminator sequence associated Leucyl | 71 |
| spinosa | | aminopeptidase | |
| Saccharopolyspora | T3 | Terminator sequence associated cytochrome P450 | 72 |
| spinosa | | hydroxylase | |
| Saccharopolyspora | T4 | Terminator sequence associated F0F1 ATP synthase | 73 |
| spinosa | | subunit beta | |
| Saccharopolyspora | T5 | Terminator sequence associated FAD-linked | 74 |
| erythraea | | oxidoreductase | |
| Saccharopolyspora | T6 | Terminator sequence associated | 75 |
| erythraea | | phosphoribosyltransferase | |
| Saccharopolyspora | T7 | Terminator sequence associated ATP-binding | 76 |
| erythraea | | protein | |
| Saccharopolyspora | T8 | Terminator sequence associated 50s Ribosomal | 77 |
| erythraea | | protein L32 | |
| Saccharopolyspora | T9 | Terminator sequence associated tRNA-Arg | 78 |
| erythraea | | | |
| Saccharopolyspora | T11 | Terminator sequence associated lsr2 | 79 |
| erythraea | | | |
| Saccharopolyspora | T12 | Terminator sequence associated AraC | 80 |
| erythraea | | | |

Reporter genes

| | | | |
|---|---|---|---|
| Artificial sequence | DasherGFP | codon optimized reporter gene DasherGFP | 81 |
| | reporter gene | | |
| Artificial sequence | PaprikaRFP | codon optimized reporter gene PaprikaRFP | 82 |
| | reporter gene | | |
| Artificial sequence | gusA reporter gene | codon optimized reporter gene gusA | 83 |
| Artificial sequence | DasherGFP | DasherGFP reporter protein | 143 |
| Artificial sequence | PaprikaRFP | PaprikaRFP reporter protein | 144 |
| Artificial sequence | gusA | gusA reporter protein | 145 |
| Artificial sequence | Terminator | Terminator sequence used for expressing GFP or | 150 |
| | sequence for | RFP reporter gene | |
| | GFP/RFP | | |

Integrases related sequences

| | | | |
|---|---|---|---|
| S. endophytica | pCM32 integrase | Integrase in pCM32 + attP sequence | 84 |
| | gene | | |
| S. endophytica | pCM32 integrase | Protein sequence of Integrase in plasmid pCM32 | 85 |
| | protein | | |
| S. endophytica | attP in pCM32 | attP site sequence in pCM32 | 167 |
| S. erythraea | pSE101 integrase | Integrase in pSE101 + attP sequence | 86 |
| | gene | | |
| S. erythraea | pSE101 integrase | Protein sequence of Integrase in plasmid pSE101 | 87 |
| | protein | | |
| S. erythraea | attP in pSE101 | attP site sequence in pSE101 | 168 |
| S. erythraea | pSE211 integrase | Integrase in pSE211 + attP sequence | 88 |
| | gene | | |
| S. erythraea | pSE211 integrase | Protein sequence of Integrase in plasmid pSE211 | 89 |
| | protein | | |
| S. erythraea | attP in pSE211 | attP site sequence in pSE211 | 169 |
| S. spinosa | pSE101 homolog | pSE101 integrase homolog gene in S. spinosa + attP | 90 |
| | integrase gene | sequence | |
| S. spinosa | pSE101 homolog | Protein sequence of pSE101 integrase homolog gene | 91 |
| | integrase protein | in S. spinosa | |
| S. spinosa | attP pSE101 | attP site sequence in pSE101 homolog construct | 170 |
| | homolog | | |
| S. spinosa | pSE211 homolog | pSE211 integrase homolog gene in S. spinosa + attP | 92 |
| | integrase gene | sequence | |
| S. spinosa | pSE211 homolog | Protein sequence of pSE211 integrase homolog gene | 93 |
| | integrase protein | in S. spinosa | |
| S. spinosa | attP pSE211 | attP site sequence in pSE211 homolog construct | 171 |
| | homolog | | |

Origins and elements of replication

| | | | |
|---|---|---|---|
| S. erythraea | origin of | putative chromosomal origin of replication from S. erythraea | 94 |
| | replication in S. erythraea | | |
| S. erythraea | pSE101 AICE | Actinomycete Integrative and Conjugative Element | 95 |
| | element | (AICEs) in replicating plasmid pSE101 | |
| S. erythraea | pSE211 AICE | Actinomycete Integrative and Conjugative Elements | 96 |
| | element | (AICEs) in replicating plasmid pSE211 | |

Ribosomal Binding Sites (RBS) sequences

| | | | |
|---|---|---|---|
| S. spinosa | RBS1 | RBS associated with PermE* | 97 |
| S. spinosa | RBS2 | RBS associated with spnA (polyketide synthase | 98 |
| | | loading & extender module1) | |
| S. spinosa | RBS3 | RBS associated with spnC (polyketide synthase | 99 |
| | | extender modules 3-4) | |
| S. spinosa | RBS4 | RBS associated with spnO (putative NDP-hexose- | 100 |
| | | 2,3-dehydratase) | |
| S. spinosa | RBS5 | RBS associated with gdh (gdh (dTDP-glucose 4,6- | 101 |
| | | dehydratase)) | |
| S. spinosa | RBS6 | RBS associated with linker_A (aldehyde | 102 |
| | | dehydrogenase, AldA) | |
| S. spinosa | RBS7 | RBS associated with linker_B (acetolactate | 103 |
| | | synthase) | |
| S. spinosa | RBS8 | RBS associated with linker_C | 104 |
| S. spinosa | RBS9 | RBS associated with linker_D | 105 |
| S. spinosa | RBS10 | RBS associated with gtt (Glucose-1-phosphate | 106 |
| | | thymidylyltransferase 1) | |
| S. spinosa | RBS11 | RBS associated with TDH (Glyceraldehyde-3- | 107 |
| | | phosphate dehydrogenase) | |
| S. spinosa | RBS12 | RBS associated with BioBrick_1 | 108 |
| S. spinosa | RBS13 | RBS associated with BioBrick_2 | 109 |
| S. spinosa | RBS14 | RBS associated with GroES (Molecular chaperon | 110 |
| | | GroES) | |
| S. spinosa | RBS15 | RBS associated with GroEL (Molecular chaperon | 111 |
| | | GroEL) | |
| S. spinosa | RBS16 | RBS associated with IF-1 (Translation initiation | 112 |
| | | factor IF-1) | |
| S. spinosa | RBS17 | RBS associated with XNR_1700 (Periplasmic | 113 |
| | | murein peptide-binding protein precursor) | |
| S. spinosa | RBS18 | RBS associated with S20 (30s ribosomal protein | 114 |
| | | S20) | |
| S. spinosa | RBS19 | RBS associated with S12 (ribosomal protein S12) | 115 |
| S. spinosa | RBS20 | RBS associated with S12 (ribosomal protein S12) | 116 |
| S. spinosa | RBS21 | RBS associated with DnaK (Hsp 70) | 117 |
| S. spinosa | RBS22 | RBS associated with elongation factor Tu | 118 |
| S. spinosa | RBS23 | RBS associated with F0F1 ATP synthase subunit | 119 |
| | | beta | |
| S. spinosa | RBS24 | RBS associated with molecular chaperone DnaK | 120 |
| S. spinosa | RBS25 | RBS associated with phage shock protein A, PspA | 121 |
| S. spinosa | RBS26 | RBS associated with 2-oxoglutarate decarboxylase | 122 |
| S. spinosa | RBS27 | RBS associated with 5- | 123 |
| | | methyltetrahydropteroyltriglutamate homocysteine | |
| | | methyltransferase | |
| S. spinosa | RBS28 | RBS associated with 50S ribosomal protein L7/L12 | 124 |
| S. spinosa | RBS29 | RBS associated with DNA-directed RNA | 125 |
| | | polymerase subunit alpha | |
| S. spinosa | RBS30 | RBS associated with 30S ribosomal protein S5 | 126 |
| S. spinosa | RBS31 | RBS associated with DnaK (6929) | 127 |

Transposon sequences

| | | | |
|---|---|---|---|
| Artificial sequence | Loss-of-Function | transposon mutagenesis payload sequence LoF | 128 |
| | (LoF) transposon | | |
| Artificial sequence | Gain-of-Function | transposon mutagenesis payload sequence Gain of | 129 |
| | (GoF) transposon | Function with a promoter | |
| Artificial sequence | Gain-of-Function | transposon mutagenesis payload sequence Gain of | 130 |
| | recyclable | Function with a counter-selection marker | |
| | transposon | | |
| Artificial sequence | Gain-of-Function | transposon mutagenesis payload sequence Gain of | 131 |
| | solubility tag | Function with a solubility tag | |
| | transposon | | |
| Artificial sequence | solubility tag | GST solubility tag sequence that can be included in | 166 |
| | | a transposon construct | |

Neutral Site sequences

| | | | |
|---|---|---|---|
| S. spinosa | SS_NeutralSite_1 | S. spinosa neutral site 1 | 132 |
| S. spinosa | SS_NeutralSite_2 | S. spinosa neutral site 2 | 133 |
| S. spinosa | SS_NeutralSite_3 | S. spinosa neutral site 3 | 134 |
| S. spinosa | SS_NeutralSite_4 | S. spinosa neutral site 4 | 135 |
| S. spinosa | SS_NeutralSite_5 | S. spinosa neutral site 5 | 136 |
| S. spinosa | SS_NeutralSite_6 | S. spinosa neutral site 6 | 137 |
| S. spinosa | SS_NeutralSite_7 | S. spinosa neutral site 7 | 138 |
| S. spinosa | SS_NeutralSite_8 | S. spinosa neutral site 8 | 139 |
| S. spinosa | SS_NeutralSite_9 | S. spinosa neutral site 9 | 140 |
| S. spinosa | SS_NeutralSite_10 | S. spinosa neutral site 10 | 141 |
| S. spinosa | SS_NeutralSite_11 | S. spinosa neutral site 11 | 142 |

Selection and Counter-Selection Markers

| | | | |
|---|---|---|---|
| Artificial sequence | SacB gene | sacB gene sequence, codon optimized for S. spinosa | 146 |
| Artificial sequence | Mutated pheS | S. erythraea gene sequence used with mutations | 147 |
| | gene (S. erythraea) | described in Miyazaki 2015 | |
| Artificial sequence | Mutated pheS | S. spinosa gene sequence used with mutations | 148 |
| | gene (S. spinosa) | described in Miyazaki 2015 | |
| S. erythraea | ermE promoter | ermE promoter sequence driving expression of ermE | 149 |
| | sequence | selection gene | |
| Artificial sequence | aac(3)IV | aac(3)IV protein conffering resistance to apramycin | 151 |
| Artificial sequence | aacC1 | aacC1 protein conferring resistance to Gentamycin | 152 |
| Artificial sequence | aacC8 | aacC8 protein conferring resistance to Neomycin B | 153 |
| Artificial sequence | aadA | aadA protein conferring resistance to | 154 |
| | | Spectinomycin/Streptomycin | |
| Artificial sequence | ble | ble protein conferring resistance to Bleomycin | 155 |
| Artificial sequence | cat | cat protein conferring resistance to | 156 |
| | | Chloramphenicol | |
| Artificial sequence | ermE | ermE protein conferring resistance to Erythromycin | 157 |
| Artificial sequence | hyg | hyg protein conferring resistance to Hygromycin | 158 |
| Artificial sequence | neo | neo protein conferring resistance to Kanamycin | 159 |
| Artificial sequence | amdSYM | Counter selection marker amdSYM gene | 160 |
| Artificial sequence | tetA | Counter selection marker tetA gene | 161 |
| Artificial sequence | lacY | Counter selection marker lacY gene | 162 |
| Artificial sequence | sacB | Counter selection marker sacB gene | 163 |
| Artificial sequence | pheS, S. erythraea | Counter selection marker pheS gene derived from S. erythraea | 164 |
| Artificial sequence | pheS, | Counter selection marker pheS gene derived from | 165 |
| | Corynebacterium | Corynebacterium | |

Integrase Attachment Sites (att sites)

| | | | |
|---|---|---|---|
| Saccharopolyspora | — | attP site in pCM32 | 167 |
| endophytica | | | |
| Saccharopolyspora | — | attP site in pSE101 | 168 |
| erythraea | | | |
| Saccharopolyspora | — | attP site in pSE211 | 169 |
| erythraea | | | |
| S. spinosa | — | attP site in pSE101 homolog | 170 |
| S. spinosa | — | attP site in pSE211 homolog | 171 |

### Numbered Embodiments of the Disclosure

Notwithstanding the appended clauses, the disclosure sets forth the following numbered embodiments.

### High-Throughput Genomic Engineering to Evolve a Saccharopolyspora sp.

1. A high-throughput (HTP) method of genomic engineering to evolve a Saccharopolyspora sp. microbe to acquire a desired phenotype, comprising:
a. perturbing the genomes of an initial plurality of Saccharopolyspora microbes having the same genomic strain background, to thereby create an initial HTP genetic design Saccharopolyspora strain library comprising individual Saccharopolyspora strains with unique genetic variations;
b. screening and selecting individual Saccharopolyspora strains of the initial HTP genetic design Saccharopolyspora strain library for the desired phenotype;
c. providing a subsequent plurality of Saccharopolyspora microbes that each comprise a unique combination of genetic variation, said genetic variation selected from the genetic variation present in at least two individual Saccharopolyspora strains screened in the preceding step, to thereby create a subsequent HTP genetic design Saccharopolyspora strain library;
d. screening and selecting individual Saccharopolyspora strains of the subsequent HTP genetic design Saccharopolyspora strain library for the desired phenotype; and
e. repeating steps c)-d) one or more times, in a linear or non-linear fashion, until a Saccharopolyspora microbe has acquired the desired phenotype, wherein each subsequent iteration creates a new HTP genetic design Saccharopolyspora strain library comprising individual Saccharopolyspora strains harboring unique genetic variations that are a combination of genetic variation selected from amongst at least two individual Saccharopolyspora strains of a preceding HTP genetic design Saccharopolyspora strain library.
   1.1 A high-throughput (HTP) method of genomic engineering to evolve a Saccharopolyspora sp. microbe to acquire a desired phenotype, comprising:
a. obtaining an initial plurality of Saccharopolyspora microbes comprising individual Saccharopolyspora strains with unique genetic variations, to thereby create an initial HTP genetic design Saccharopolyspora strain library;
b. screening and selecting individual Saccharopolyspora strains of the initial HTP genetic design Saccharopolyspora strain library for the desired phenotype;
c. providing a subsequent plurality of Saccharopolyspora microbes that each comprise a unique combination of genetic variation, said genetic variation selected from the genetic variation present in at least two individual Saccharopolyspora strains screened in the preceding step, to thereby create a subsequent HTP genetic design Saccharopolyspora strain library;
d. screening and selecting individual Saccharopolyspora strains of the subsequent HTP genetic design Saccharopolyspora strain library for the desired phenotype; and
e. repeating steps c)-d) one or more times, in a linear or non-linear fashion, until a Saccharopolyspora microbe has acquired the desired phenotype, wherein each subsequent iteration creates a new HTP genetic design Saccharopolyspora strain library comprising individual Saccharopolyspora strains harboring unique genetic variations that are a combination of genetic variation selected from amongst at least two individual Saccharopolyspora strains of a preceding HTP genetic design Saccharopolyspora strain library.
   1.2 The HTP method of clause 1.1, wherein the initial plurality of Saccharopolyspora microbes comprising individual Saccharopolyspora strains with unique genetic variations are produced by perturbing the genomes of an initial plurality of Saccharopolyspora microbes having the same genomic strain background.
   2. The HTP method of genomic engineering according to clause 1 to 1.2, wherein the function and/or identity of the genes that contain the genetic variations are not considered before the genetic variations are combined in step (b).
   3. The HTP method of genomic engineering according to any one of clauses 1-2, wherein at least one genetic variation to be combined is not in a genomic region that contains repeating segments of encoding DNA modules.
   4. The HTP method of genomic engineering according to claim 1, wherein the subsequent plurality of Saccharopolyspora microbes that each comprises a unique combination of genetic variations in step (c) are produced by:
   1) introducing a plasmid into an individual Saccharopolyspora strain belonging to the initial HTP genetic design Saccharopolyspora strain library, wherein the plasmid comprises a selection marker, a counterselection marker, a DNA fragment having homology to the genomic locus of the base Saccharopolyspora strain, and plasmid backbone sequence, wherein the DNA fragment has a genetic variation derived from another individual Saccharopolyspora strain also belonging to the initial HTP genetic design Saccharopolyspora strain library;
   2) selecting for Saccharopolyspora strains with integration event based on the presence of the selection marker in the genome;
   3) selecting for Saccharopolyspora strains having the plasmid backbone looped out based on the absence of the counterselection marker gene.
   5. The HTP method of any one of clauses 1-4, wherein the plasmid does not comprise a temperature sensitive replicon.
   6. The HTP method of any one of clauses 1-5, wherein the selection step (3) is performed without replication of the integrated plasmid.
   7. The HTP method of genomic engineering according to any one of clauses 1-6, wherein the initial HTP genetic design Saccharopolyspora strain library comprises at least one library selected from the group consisting of a promoter swap microbial strain library, SNP swap microbial strain library, start/stop codon microbial strain library, optimized sequence microbial strain library, a terminator swap microbial strain library, a transposon mutagenesis microbial strain diversity library, a ribosomal binding site microbial strain library, an anti-metabolite/fermentation product resistance library, a termination insertion microbial strain library, and any combination thereof.
   8. The HTP method of genomic engineering according to any one of clauses 1-7, wherein the subsequent HTP genetic design Saccharopolyspora strain library is a full combinatorial Saccharopolyspora strain library of the initial HTP genetic design microbial strain library.
   9. The HTP method of genomic engineering according to any one of clauses 1-8, wherein the subsequent HTP genetic design Saccharopolyspora strain library is a subset of a full combinatorial Saccharopolyspora strain library derived from the genetic variations in the initial HTP genetic design Saccharopolyspora strain library.
   10. The HTP method of genomic engineering according to clause any one of clauses 1-9, wherein the subsequent HTP genetic design derived from the genetic variations in strain library is a full combinatorial microbial strain library derived from the genetic variations in a preceding HTP genetic design Saccharopolyspora strain library.
   11. The HTP method of genomic engineering according to any one of clauses 1-10, wherein the subsequent HTP genetic design Saccharopolyspora strain library is a subset of a full combinatorial Saccharopolyspora strain library derived from the genetic variations in a preceding HTP genetic design Saccharopolyspora strain library.
   12. The HTP method of genomic engineering according to any one of clauses 1-11, wherein perturbing the genome comprises utilizing at least one method selected from the group consisting of: random mutagenesis, targeted sequence insertions, targeted sequence deletions, targeted sequence replacements, transposon mutagenesis, and any combination thereof.
   13. The HTP method of genomic engineering according to any one of clauses 1-12, wherein the initial plurality of Saccharopolyspora microbes comprise unique genetic variations derived from a production Saccharopolyspora strain.
   14. The HTP method of genomic engineering according to any one of clauses 1-13, wherein the initial plurality of Saccharopolyspora microbes comprise production strain microbes denoted S₁Gen₁ and any number of subsequent microbial generations derived therefrom denoted SnGenn.
   15. The HTP method of genomic engineering according to any one of clauses 1-14, wherein the step c comprises rapidly consolidating the genetic variations by using protoplast fusion techniques.
   16. The HTP method of genomic engineering according to any one of clauses 1-15, wherein the initial HTP genetic design Saccharopolyspora strain library or the subsequent HTP genetic design Saccharopolyspora strain library comprises a promoter swap microbial strain library.
   17. The HTP method of genomic engineering according to clause 16, wherein the promoter swap microbial strain library comprises at least one promoter with a nucleotide sequence selected from SEQ ID Nos. 1 to 69 and 172 to 175.
   18. The HTP method of genomic engineering according to clause any one of clauses 1-17, wherein the initial HTP genetic design Saccharopolyspora strain library or the subsequent HTP genetic design Saccharopolyspora strain library comprises a SNP swap microbial strain library.
   19. The HTP method of genomic engineering according to clause any one of clauses 1-18, wherein the initial HTP genetic design Saccharopolyspora strain library or the subsequent HTP genetic design Saccharopolyspora strain library comprises a terminator swap microbial strain library.
   20. The HTP method of genomic engineering according to clause 19, wherein the terminator swap microbial strain library comprises at least one terminator with a nucleotide sequence selected from SEQ ID Nos. 70 to 80.
   21. The HTP method of genomic engineering according to clause any one of clauses 1-20, wherein the initial HTP genetic design Saccharopolyspora strain library or the subsequent HTP genetic design Saccharopolyspora strain library comprises a transposon mutagenesis microbial strain diversity library.
   22. The HTP method of genomic engineering according to clause 21, wherein the initial HTP genetic design Saccharopolyspora strain library or the subsequent HTP genetic design Saccharopolyspora strain library comprises a Loss-of-Function (LoF) transposon and/or a Gain-of-Function (GoF) transposon.
   23. The HTP method of genomic engineering according to clause 22, wherein the GoF transposon comprises a solubility tag, a promoter, and/or a counter-selection marker.
   24. The HTP method of genomic engineering according to clause any one of clauses 1-23, wherein the initial HTP genetic design Saccharopolyspora strain library or the subsequent HTP genetic design Saccharopolyspora strain library comprises a ribosomal binding site microbial strain library.
   25. The HTP method of genomic engineering according to clause 24, wherein ribosomal binding site microbial strain library comprises at least one ribosomal binding site (RBS) with a nucleotide sequence selected from SEQ ID Nos. 97 to 127.
   26. The HTP method of genomic engineering according to clause any one of clauses 1-25, wherein the initial HTP genetic design Saccharopolyspora strain library or the subsequent HTP genetic design Saccharopolyspora strain library comprises an anti-metabolite/fermentation product resistance library.
   27. The HTP method of genomic engineering according to clause 26, wherein the anti-metabolite/fermentation product resistance library comprises a Saccharopolyspora strain resistance to a molecule involved in spinosyn synthesis in Saccharopolyspora.
   Generating a SNP sawp Saccharopolyspora Strain Library
   28. A method for generating a SNP swap Saccharopolyspora strain library, comprising the steps of:
a. providing a reference Saccharopolyspora strain and a second Saccharopolyspora strain, wherein the second Saccharopolyspora strain comprises a plurality of identified genetic variations selected from single nucleotide polymorphisms, DNA insertions, and DNA deletions, which are not present in the reference Saccharopolyspora strain; and
b. perturbing the genome of either the reference Saccharopolyspora strain, or the second Saccharopolyspora strain, to thereby create an initial SNP swap Saccharopolyspora strain library comprising a plurality of individual Saccharopolyspora strains with unique genetic variations found within each strain of said plurality of individual Saccharopolyspora strains, wherein each of said unique genetic variations corresponds to a single genetic variation selected from the plurality of identified genetic variations between the reference Saccharopolyspora strain and the second Saccharopolyspora strain.
   29. The method for generating a SNP swap Saccharopolyspora strain library according to clause 28, wherein the genome of the reference Saccharopolyspora strain is perturbed to add one or more of the identified single nucleotide polymorphisms, DNA insertions, or DNA deletions, which are found in the second Saccharopolyspora strain.
   30. The method for generating a SNP swap Saccharopolyspora strain library according to any one of clauses 28-29, wherein the genome of the second Saccharopolyspora strain is perturbed to remove one or more of the identified single nucleotide polymorphisms, DNA insertions, or DNA deletions, which are not found in the reference Saccharopolyspora strain.
   31. The method for generating a SNP swap Saccharopolyspora strain library according to any one of clauses 28-30, wherein the resultant plurality of individual Saccharopolyspora strains with unique genetic variations, together comprise a full combinatorial library of all the identified genetic variations between the reference Saccharopolyspora strain and the second Saccharopolyspora strain.
   32. The method for generating a SNP swap Saccharopolyspora strain library according to any one of clauses 28-31, wherein the resultant plurality of individual Saccharopolyspora strains with unique genetic variations, together comprise a subset of a full combinatorial library of all the identified genetic variations between the reference Saccharopolyspora strain and the second Saccharopolyspora strain.

### Rehabilitating and Improving the Phenotypic Performance of a Production Saccharopolyspora Strain

33. A method for rehabilitating and improving the phenotypic performance of a production Saccharopolyspora strain, comprising the steps of:
a. providing a parental lineage Saccharopolyspora strain and a production Saccharopolyspora strain derived therefrom, wherein the production Saccharopolyspora strain comprises a plurality of identified genetic variations selected from single nucleotide polymorphisms, DNA insertions, and DNA deletions, not present in the parental lineage Saccharopolyspora strain;
b. perturbing the genome of either the parental lineage Saccharopolyspora strain, or the production Saccharopolyspora strain, to thereby create an initial Saccharopolyspora strain library. Wherein each strain in the initial library comprises a unique genetic variation from the plurality of identified genetic variations between the parental lineage Saccharopolyspora strain and the production Saccharopolyspora strain;
c. screening and selecting individual Saccharopolyspora strains of the initial SNP swap Saccharopolyspora strain library for phenotype performance improvements over a reference Saccharopolyspora strain, thereby identifying unique genetic variations that confer phenotypic performance improvements;
d. providing a subsequent plurality of microbes that each comprise a combination of unique genetic variation from the variations present in at least two individual Saccharopolyspora strains screened in the preceding step, to thereby create a subsequent library of Saccharopolyspora strains;
e. screening and selecting individual strains of the subsequent strain library for phenotypic performance improvements over the reference Saccharopolyspora strain, thereby identifying unique combinations of genetic variation that confer additional phenotypic performance improvements; and
f. repeating steps d)-e) one or more times, in a linear or non-linear fashion, until a Saccharopolyspora strain exhibits a desired level of improved phenotypic performance compared to the phenotypic performance of the production Saccharopolyspora strain, wherein each subsequent iteration creates a new library of Saccharopolyspora strains—where each strain in the new library comprises genetic variations that are a combination of genetic variations selected from amongst at least two individual Saccharopolyspora strains of a preceding library.
   34. The method for rehabilitating and improving the phenotypic performance of a production Saccharopolyspora strain according to clause 33, wherein the initial library of Saccharopolyspora strains is a full combinatorial library comprising all of the identified genetic variations between the parental lineage Saccharopolyspora strain and the production Saccharopolyspora strain.
   35. The method for rehabilitating and improving the phenotypic performance of a production Saccharopolyspora strain according to any one of clauses 33-34, wherein the initial library of Saccharopolyspora strains is a subset of a full combinatorial library comprising a subset of the identified genetic variations between the reference parental lineage Saccharopolyspora strain and the production Saccharopolyspora strain.
   36. The method for rehabilitating and improving the phenotypic performance of a production Saccharopolyspora strain according to any one of clauses 33-35, wherein the subsequent library of Saccharopolyspora strains is a full combinatorial library of the initial library.
   37. The method for rehabilitating and improving the phenotypic performance of a production Saccharopolyspora strain according to any one of clauses 33-36, wherein the subsequent library of Saccharopolyspora strains is a full combinatorial library of the initial library.
   38. The method for rehabilitating and improving the phenotypic performance of a production Saccharopolyspora strain according to any one of clauses 33-37, wherein the subsequent library of Saccharopolyspora strains is a full combinatorial library of a preceding library.
   39. The method for rehabilitating and improving the phenotypic performance of a production Saccharopolyspora strain according to any one of clauses 33-38, wherein the subsequent library of Saccharopolyspora strains is a subset of a full combinatorial library of a preceding library.
   40. The method for rehabilitating and improving the phenotypic performance of a production Saccharopolyspora strain according to any one of clauses 33-39, wherein the genome of the parental lineage Saccharopolyspora strain is perturbed to add one or more of the identified single nucleotide polymorphisms, DNA insertions, or DNA deletions, which are found in the production Saccharopolyspora strain.
   41. The method for rehabilitating and improving the phenotypic performance of a production Saccharopolyspora strain according to any one of clauses 33-40, wherein the genome of the production Saccharopolyspora strain is perturbed to remove one or more of the identified single nucleotide polymorphisms, DNA insertions, or DNA deletions, which are not found in the parental lineage Saccharopolyspora strain.
   42. The method for rehabilitating and improving the phenotypic performance of a production Saccharopolyspora strain according to any one of clauses 33-41, wherein perturbing the genome comprises utilizing at least one method selected from the group consisting of: random mutagenesis, targeted sequence insertions, targeted sequence deletions, targeted sequence replacements, and combinations thereof.
   43. The method for rehabilitating and improving the phenotypic performance of a production Saccharopolyspora strain according to any one of clauses 33-42, wherein steps d)-e) are repeated until the phenotypic performance of a Saccharopolyspora strain of a subsequent library exhibits at least a 10% increase in a measured phenotypic variable compared to the phenotypic performance of the production Saccharopolyspora strain.
   44. The method for rehabilitating and improving the phenotypic performance of a production Saccharopolyspora strain according to any one of clauses 33-43, wherein steps d)-e) are repeated until the phenotypic performance of a Saccharopolyspora strain of a subsequent library exhibits at least a one-fold increase in a measured phenotypic variable compared to the phenotypic performance of the production Saccharopolyspora strain.
   45. The method for rehabilitating and improving the phenotypic performance of a production Saccharopolyspora strain according to any one of clauses 33-44, wherein the improved phenotypic performance of step f) is selected from the group consisting of: volumetric productivity of a product of interest, specific productivity of a product of interest, yield of a product of interest, titer of a product of interest, and combinations thereof.
   46. The method for rehabilitating and improving the phenotypic performance of a production Saccharopolyspora strain according to any one of clauses 33-45, wherein the improved phenotypic performance of step f) is: increased or more efficient production of a product of interest, said product of interest selected from the group consisting of: a small molecule, enzyme, peptide, amino acid, organic acid, synthetic compound, fuel, alcohol, primary extracellular metabolite, secondary extracellular metabolite, intracellular component molecule, and combinations thereof.
   47. The method for rehabilitating and improving the phenotypic performance of a production Saccharopolyspora strain according to clause 46, wherein the product of interest is selected from the group consisting of a spinosyn, spinosad, spinetoram, genistein, choline oxidase, a coumamidine compound, erythromycin, ivermectin aglycone, a HMG-CoA reductase inhibitor, a carboxylic acid isomer, alpha-methyl methionine, thialysine, alpha-ketobytarate, aspartate hydoxymate, azaserine, 5-fuoroindole, beta-hydroxynorvaline, cerulenin, purine, pyrimidine, and analogs thereof.
   48. The method for rehabilitating and improving the phenotypic performance of a production Saccharopolyspora strain according to clause 46, wherein the spinosyn is spinosyn A, spinosyn D, spinosyn J, spinosyn L, or combinations thereof.
   49. The method for rehabilitating and improving the phenotypic performance of a production Saccharopolyspora strain according to any one of clauses 33-48, wherein the identified genetic variations further comprise artificial promoter swap genetic variations from a promoter swap library.
   50. The method for rehabilitating and improving the phenotypic performance of a production Saccharopolyspora strain according to any one of clauses 33-49, further comprising engineering the genome of at least one microbial strain of either the initial library of Saccharopolyspora strains, or a subsequent library of Saccharopolyspora strains, to comprise one or more promoters from a promoter ladder operably linked to an endogenous Saccharopolyspora target gene.
   51. The method for rehabilitating and improving the phenotypic performance of a production Saccharopolyspora strain according to any one of clauses 33-50, wherein the strain library comprises at least one library selected from the group consisting of a promoter swap microbial strain library, SNP swap microbial strain library, start/stop codon microbial strain library, optimized sequence microbial strain library, a terminator swap microbial strain library, a transposon mutagenesis microbial strain diversity library, a ribosomal binding site microbial strain library, an anti-metabolite/fermentation product resistance library, a termination insertion microbial strain library, and any combination thereof.
   52. The method for rehabilitating and improving the phenotypic performance of a production Saccharopolyspora strain according to clause 51, wherein the strain library comprises at least one library selected from the group consisting of:
   1) a promoter swap microbial strain library comprising at least one promoter having a sequence selected from SEQ ID No. 1-69;
   2) a terminator swap microbial strain library comprising at least one terminator having a sequence selected from SEQ ID Nos. 70 to 80; and
   3) a ribosomal binding site (RBS) library comprising at least one RBS having a sequence selected from SEQ ID Nos. 97 to 127.

### Generating a Promoter Swap Saccharopolyspora Strain Library and Using the Same for Improving the Phenotypic Performance of a Production Saccharopolyspora Strain

53. A method for generating a promoter swap Saccharopolyspora strain library, said method comprising the steps of:
a. providing a plurality of target genes endogenous to a base Saccharopolyspora strain, and a promoter ladder, wherein said promoter ladder comprises a plurality of promoters exhibiting different expression profiles in the base Saccharopolyspora strain; and
b. engineering the genome of the base Saccharopolyspora strain, to thereby create an initial promoter swap Saccharopolyspora strain library comprising a plurality of individual Saccharopolyspora strains with unique genetic variations found within each strain of said plurality of individual Saccharopolyspora strains, wherein each of said unique genetic variations comprises one or more of the promoters from the promoter ladder operably linked to one of the target genes endogenous to the base Saccharopolyspora strain.
   54. The method for generating a promoter swap Saccharopolyspora strain library according to clause 53, wherein at least one of the plurality of promoters comprises a promoter having a sequence selected from SEQ ID No. 1-69.
   55. A promoter swap method for improving the phenotypic performance of a production Saccharopolyspora strain, comprising the steps of:
a. providing a plurality of target genes endogenous to a base Saccharopolyspora strain, and a promoter ladder, wherein said promoter ladder comprises a plurality of promoters exhibiting different expression profiles in the base Saccharopolyspora strain;
b. engineering the genome of the base Saccharopolyspora strain, to thereby create an initial promoter swap Saccharopolyspora strain library comprising a plurality of individual Saccharopolyspora strains with unique genetic variations found within each strain of said plurality of individual Saccharopolyspora strains, wherein each of said unique genetic variations comprises one or more of the promoters from the promoter ladder operably linked to one of the target genes endogenous to the base Saccharopolyspora strain;
c. screening and selecting individual Saccharopolyspora strains of the initial promoter swap Saccharopolyspora strain library for phenotypic performance improvements over a reference Saccharopolyspora strain, thereby identifying unique genetic variations that confer the phenotypic performance improvements;
d. providing a subsequent plurality of Saccharopolyspora microbes that each comprise a combination of unique genetic variations from the genetic variations present in at least two individual Saccharopolyspora strains screened in the preceding step, to thereby create a subsequent promoter swap Saccharopolyspora strain library;
e. screening and selecting individual Saccharopolyspora strains of the subsequent promoter swap Saccharopolyspora strain library for the desired phenotypic performance improvements over the reference E. coli strain, thereby identifying unique combinations of genetic variation that confer additional phenotypic performance improvements; and
f. repeating steps d)-e) one or more times, in a linear or non-linear fashion, until a Saccharopolyspora strain exhibits a desired level of improved phenotypic performance compared to the phenotypic performance of the production Saccharopolyspora strain, wherein each subsequent iteration creates a new promoter swap Saccharopolyspora strain library of Saccharopolyspora strains, wherein each strain in the new library comprises genetic variations that are a combination of genetic variations selected from amongst at least two individual Saccharopolyspora strains of a preceding promoter swap Saccharopolyspora strain library.
   56. The promoter swap method for improving the phenotypic performance of a production Saccharopolyspora strain according to clause 55, wherein the subsequent promoter swap Saccharopolyspora strain library is a full combinatorial library of the initial promoter swap Saccharopolyspora strain library.
   57. The promoter swap method for improving the phenotypic performance of a production Saccharopolyspora strain according to any one of clauses 55-56, wherein the subsequent promoter swap Saccharopolyspora strain library is a full combinatorial library of the initial promoter swap Saccharopolyspora strain library.
   58. The promoter swap method for improving the phenotypic performance of a production Saccharopolyspora strain according to any one of clauses 55-57, wherein the subsequent promoter swap Saccharopolyspora strain library is a subset of a full combinatorial library of the initial promoter swap Saccharopolyspora strain library.
   59. The promoter swap method for improving the phenotypic performance of a production Saccharopolyspora strain according to any one of clauses 55-58, wherein the subsequent promoter swap Saccharopolyspora strain library is a full combinatorial library of a preceding promoter swap Saccharopolyspora strain library.
   60. The promoter swap method for improving the phenotypic performance of a production Saccharopolyspora strain according to any one of clauses 55-59, wherein the subsequent promoter swap Saccharopolyspora strain library is a subset of a full combinatorial library of a preceding promoter swap Saccharopolyspora strain library.
   61. The promoter swap method for improving the phenotypic performance of a production Saccharopolyspora strain according to any one of clauses 55-60, wherein steps d)-e) are repeated until the phenotypic performance a Saccharopolyspora strain of a subsequent promoter swap Saccharopolyspora strain library exhibits at least a 10% increase in a measured phenotypic variable compared to the phenotypic performance of the production Saccharopolyspora strain.
   62. The promoter swap method for improving the phenotypic performance of a production Saccharopolyspora strain according to any one of clauses 55-61, wherein steps d)-e) are repeated until the phenotypic performance of a Saccharopolyspora strain of a subsequent promoter swap Saccharopolyspora strain library exhibits at least a one-fold increase in a measured phenotypic variable compared to the phenotypic performance of the production Saccharopolyspora strain.
   63. The promoter swap method for improving the phenotypic performance of a production Saccharopolyspora strain according to any one of clauses 55-62, wherein the improved phenotypic performance of step f) is selected from the group consisting of: volumetric productivity of a product of interest, specific productivity of a product of interest, yield of a product of interest, titer of a product of interest, and combinations thereof.
   64. The promoter swap method for improving the phenotypic performance of a production Saccharopolyspora strain according to any one of clauses 55-63, wherein the improved phenotypic performance of step f) is: increased or more efficient production of a product of interest, said product of interest selected from the group consisting of: a small molecule, enzyme, peptide, amino acid, organic acid, synthetic compound, fuel, alcohol, primary extracellular metabolite, secondary extracellular metabolite, intracellular component molecule, and combinations thereof.
   65. The promoter swap method for improving the phenotypic performance of a production Saccharopolyspora strain according to clause 64, wherein the product of interest is selected from the group consisting of a spinosyn, spinosad, spinetoram, genistein, choline oxidase, a coumamidine compound, erythromycin, ivermectin aglycone, a HMG-CoA reductase inhibitor, a carboxylic acid isomer, alpha-methyl methionine, thialysine, alpha-ketobytarate, aspartate hydoxymate, azaserine, 5-fuoroindole, beta-hydroxynorvaline, cerulenin, purine, pyrimidine, and analogs thereof.
   66. The promoter swap method for improving the phenotypic performance of a production Saccharopolyspora strain according to clause 65, wherein the spinosyn is spinosyn A, spinosyn D, spinosyn J, spinosyn L, or combinations thereof.
   67. The promoter swap method for improving the phenotypic performance of a production Saccharopolyspora strain according to any one of clauses 55-66, wherein the promoter ladder comprises at least one promoter with a nucleotide sequence selected from SEQ ID No. 1-69.

### Generating a Terminator Swap Saccharopolyspora Strain Library and Using the Same for Improving the Phenotypic Performance of a Production Saccharopolyspora Strain

68. A method for generating a terminator swap Saccharopolyspora strain library, comprising the steps of:
a. providing a plurality of target genes endogenous to a base Saccharopolyspora strain, and a terminator ladder, wherein said terminator ladder comprises a plurality of terminators exhibiting different expression profiles in the base Saccharopolyspora strain; and
b. engineering the genome of the base Saccharopolyspora strain, to thereby create an initial terminator swap Saccharopolyspora strain library comprising a plurality of individual Saccharopolyspora strains with unique genetic variations found within each strain of said plurality of individual Saccharopolyspora strains, wherein each of said unique genetic variations comprises one or more of the terminators from the terminator ladder operably linked to one of the target genes endogenous to the base Saccharopolyspora strain.
   69. A terminator swap method for improving the phenotypic performance of a production Saccharopolyspora strain, comprising the steps of:
a. providing a plurality of target genes endogenous to a base Saccharopolyspora strain, and a terminator ladder, wherein said terminator ladder comprises a plurality of terminators exhibiting different expression profiles in the base Saccharopolyspora strain;
b. engineering the genome of the base Saccharopolyspora strain, to thereby create an initial terminator swap Saccharopolyspora strain library comprising a plurality of individual Saccharopolyspora strains with unique genetic variations found within each strain of said plurality of individual Saccharopolyspora strains, wherein each of said unique genetic variations comprises one or more of the terminators from the terminator ladder operably linked to one of the target genes endogenous to the base Saccharopolyspora strain;
c. screening and selecting individual Saccharopolyspora strains of the initial terminator swap Saccharopolyspora strain library for phenotypic performance improvements over a reference Saccharopolyspora strain, thereby identifying unique genetic variations that confer phenotypic performance improvements;
d. providing a subsequent plurality of Saccharopolyspora microbes that each comprise a combination of unique genetic variations from the genetic variations present in at least two individual Saccharopolyspora strains screened in the preceding step, to thereby create a subsequent terminator swap Saccharopolyspora strain library;
e. screening and selecting individual Saccharopolyspora strains of the subsequent terminator swap Saccharopolyspora strain library for phenotypic performance improvements over the reference Saccharopolyspora strain, thereby identifying unique combinations of genetic variation that confer additional phenotypic performance improvements; and
f. repeating steps d)-e) one or more times, in a linear or non-linear fashion, until a Saccharopolyspora strain exhibits a desired level of improved phenotypic performance compared to the phenotypic performance of the production Saccharopolyspora strain, wherein each subsequent iteration creates a new terminator swap Saccharopolyspora strain library of microbial strains, where each strain in the new library comprises genetic variations that are a combination of genetic variations selected from amongst at least two individual Saccharopolyspora strains of a preceding library.
   70. The terminator swap method for improving the phenotypic performance of a production Saccharopolyspora strain according to clause 69, wherein the subsequent terminator swap Saccharopolyspora strain library is a full combinatorial library of the initial terminator swap Saccharopolyspora strain library.
   71. The terminator swap method for improving the phenotypic performance of a production Saccharopolyspora strain according to any one of clauses 69-70, wherein the subsequent terminator swap Saccharopolyspora strain library is a subset of a full combinatorial library of the initial terminator swap Saccharopolyspora strain library.
   72. The terminator swap method for improving the phenotypic performance of a production Saccharopolyspora strain according to any one of clauses 69-71, wherein the subsequent terminator swap Saccharopolyspora strain library is a full combinatorial library of a preceding terminator swap Saccharopolyspora strain library.
   73. The terminator swap method for improving the phenotypic performance of a production Saccharopolyspora strain according to any one of clauses 69-72, wherein the subsequent terminator swap Saccharopolyspora strain library is a subset of a full combinatorial library of a preceding terminator swap Saccharopolyspora strain library.
   74. The terminator swap method for improving the phenotypic performance of a production Saccharopolyspora strain according to any one of clauses 69-73, wherein steps d)-e) are repeated until the phenotypic performance of a Saccharopolyspora strain of a subsequent terminator swap Saccharopolyspora strain library exhibits at least a 10% increase in a measured phenotypic variable compared to the phenotypic performance of the production Saccharopolyspora strain.
   75. The terminator swap method for improving the phenotypic performance of a production Saccharopolyspora strain according to any one of clauses 69-74, wherein steps d)-e) are repeated until the phenotypic performance of a Saccharopolyspora strain of a subsequent terminator swap Saccharopolyspora strain library exhibits at least a one-fold increase in a measured phenotypic variable compared to the phenotypic performance of the production Saccharopolyspora strain.
   76. The terminator swap method for improving the phenotypic performance of a production Saccharopolyspora strain according to any one of clauses 69-75, wherein the improved phenotypic performance of step f) is selected from the group consisting of: volumetric productivity of a product of interest, specific productivity of a product of interest, yield of a product of interest, titer of a product of interest, and combinations thereof.
   77. The terminator swap method for improving the phenotypic performance of a production Saccharopolyspora strain according to any one of clauses 69-76, wherein the improved phenotypic performance of step f) is: increased or more efficient production of a product of interest, said product of interest selected from the group consisting of: a small molecule, enzyme, peptide, amino acid, organic acid, synthetic compound, fuel, alcohol, primary extracellular metabolite, secondary extracellular metabolite, intracellular component molecule, and combinations thereof.
   78. The terminator swap method for improving the phenotypic performance of a production Saccharopolyspora strain according to any one of clauses 69-77, wherein the product of interest is selected from the group consisting of a spinosyn, spinosad, spinetoram, genistein, choline oxidase, a coumamidine compound, erythromycin, ivermectin aglycone, a HMG-CoA reductase inhibitor, a carboxylic acid isomer, alpha-methyl methionine, thialysine, alpha-ketobytarate, aspartate hydoxymate, azaserine, 5-fuoroindole, beta-hydroxynorvaline, cerulenin, purine, pyrimidine, and analogs thereof.
   79. The terminator swap method for improving the phenotypic performance of a production Saccharopolyspora strain according to clause 78, wherein the spinosyn is spinosyn A, spinosyn D, spinosyn J, spinosyn L, or combinations thereof.
   80. The terminator swap method for improving the phenotypic performance of a production Saccharopolyspora strain according to any one of clauses 69-79, wherein the terminator ladder comprises at least one terminator with a nucleotide sequence selected from SEQ ID No. 70-80.

### Generating a Ribosomal Binding Site (RBS) Saccharopolyspora Strain Library and Using the Same for Improving the Phenotypic Performance of a Production Saccharopolyspora Strain

81. A method for generating a ribosomal binding site (RBS) Saccharopolyspora strain library, comprising the steps of:
a. providing a plurality of target genes endogenous to a base Saccharopolyspora strain, and a RBS ladder, wherein said RBS ladder comprises a plurality of RBSs exhibiting different expression profiles in the base Saccharopolyspora strain; and
b. engineering the genome of the base Saccharopolyspora strain, to thereby create an initial RBS Saccharopolyspora strain library comprising a plurality of individual Saccharopolyspora strains with unique genetic variations found within each strain of said plurality of individual Saccharopolyspora strains, wherein each of said unique genetic variations comprises one or more of the RBSs from the RBS ladder operably linked to one of the target genes endogenous to the base Saccharopolyspora strain.
82. A method for improving the phenotypic performance of a production Saccharopolyspora strain, comprising the steps of:
a. providing a plurality of target genes endogenous to a base Saccharopolyspora strain, and a RBS ladder, wherein said RBS ladder comprises a plurality of RBSs exhibiting different expression profiles in the base Saccharopolyspora strain;
b. engineering the genome of the base Saccharopolyspora strain, to thereby create an initial RBS Saccharopolyspora strain library comprising a plurality of individual Saccharopolyspora strains with unique genetic variations found within each strain of said plurality of individual Saccharopolyspora strains, wherein each of said unique genetic variations comprises one or more of the RBSs from the RBS ladder operably linked to one of the target genes endogenous to the base Saccharopolyspora strain;
c. screening and selecting individual Saccharopolyspora strains of the initial RBS Saccharopolyspora strain library for phenotypic performance improvements over a reference Saccharopolyspora strain, thereby identifying unique genetic variations that confer phenotypic performance improvements;
d. providing a subsequent plurality of Saccharopolyspora strains that each comprise a combination of unique genetic variations from the genetic variations present in at least two individual Saccharopolyspora strains screened in the preceding step, to thereby create a subsequent RBS Saccharopolyspora strain library;
e. screening and selecting individual Saccharopolyspora strains of the subsequent RBS Saccharopolyspora strain library for phenotypic performance improvements over the reference Saccharopolyspora strain, thereby identifying unique combinations of genetic variation that confer additional phenotypic performance improvements; and
f. repeating steps d)-e) one or more times, in a linear or non-linear fashion, until a Saccharopolyspora strain exhibits a desired level of improved phenotypic performance compared to the phenotypic performance of the production Saccharopolyspora strain, wherein each subsequent iteration creates a new RBS Saccharopolyspora strain library of microbial strains, where each strain in the new library comprises genetic variations that are a combination of genetic variations selected from amongst at least two individual Saccharopolyspora strains of a preceding library.
83. The method for improving the phenotypic performance of a production Saccharopolyspora strain according to clause 82, wherein the subsequent RBS Saccharopolyspora strain library is a full combinatorial library of the initial RBS Saccharopolyspora strain library.
84. The method for improving the phenotypic performance of a production Saccharopolyspora strain according to any one of clauses 82-83, wherein the subsequent RBS Saccharopolyspora strain library is a subset of a full combinatorial library of the initial RBS Saccharopolyspora strain library.
85. The method for improving the phenotypic performance of a production Saccharopolyspora strain according to any one of clauses 82-84, wherein the subsequent RBS Saccharopolyspora strain library is a full combinatorial library of a preceding RBS Saccharopolyspora strain library.
86. The method for improving the phenotypic performance of a production Saccharopolyspora strain according to any one of clauses 82-85, wherein the subsequent RBS Saccharopolyspora strain library is a subset of a full combinatorial library of a preceding RBS Saccharopolyspora strain library.
87. The method for improving the phenotypic performance of a production Saccharopolyspora strain according to any one of clauses 82-86, wherein steps d)-e) are repeated until the phenotypic performance of a Saccharopolyspora strain of a subsequent RBS Saccharopolyspora strain library exhibits at least a 10% increase in a measured phenotypic variable compared to the phenotypic performance of the production Saccharopolyspora strain.
88. The method for improving the phenotypic performance of a production Saccharopolyspora strain according to any one of clauses 82-87, wherein steps d)-e) are repeated until the phenotypic performance of a Saccharopolyspora strain of a subsequent RBS Saccharopolyspora strain library exhibits at least a one-fold increase in a measured phenotypic variable compared to the phenotypic performance of the production Saccharopolyspora strain.
89. The method for improving the phenotypic performance of a production Saccharopolyspora strain according to any one of clauses 82-88, wherein the improved phenotypic performance of step f) is selected from the group consisting of: volumetric productivity of a product of interest, specific productivity of a product of interest, yield of a product of interest, titer of a product of interest, and combinations thereof.
90. The method for improving the phenotypic performance of a production Saccharopolyspora strain according to any one of clauses 82-89, wherein the improved phenotypic performance of step f) is: increased or more efficient production of a product of interest, said product of interest selected from the group consisting of: a small molecule, enzyme, peptide, amino acid, organic acid, synthetic compound, fuel, alcohol, primary extracellular metabolite, secondary extracellular metabolite, intracellular component molecule, and combinations thereof.
91. The method for improving the phenotypic performance of a production Saccharopolyspora strain according to any one of clauses 82-90, wherein the product of interest is selected from the group consisting of a spinosyn, spinosad, spinetoram, genistein, choline oxidase, a coumamidine compound, erythromycin, ivermectin aglycone, a HMG-CoA reductase inhibitor, a carboxylic acid isomer, alpha-methyl methionine, thialysine, alpha-ketobytarate, aspartate hydoxymate, azaserine, 5-fuoroindole, beta-hydroxynorvaline, cerulenin, purine, pyrimidine, and analogs thereof.
92. The method for improving the phenotypic performance of a production Saccharopolyspora strain according to clause 91, wherein the spinosyn is spinosyn A, spinosyn D, spinosyn J, spinosyn L, or combinations thereof.
93. The method for improving the phenotypic performance of a production Saccharopolyspora strain according to any one of clauses 82-92, wherein the RBS ladder comprises at least one RBS with a nucleotide sequence selected from SEQ ID No. 97-127.

### Generating a Transposon Mutagenesis Saccharopolyspora Strain Library and Using the Same for Improving the Phenotypic Performance of a Production Saccharopolyspora Strain

94. A method for generating a transposon mutagenesis Saccharopolyspora strain diversity library, comprising
a) introducing a transposon into a population of cells of one or more base Saccharopolyspora strains; and
b) selecting for Saccharopolyspora strain comprising randomly integrated transposon, thereby creating an initial Saccharopolyspora strain library comprising a plurality of individual Saccharopolyspora strains with unique genetic variations found within each strain of said plurality of individual Saccharopolyspora strains, wherein each of said unique genetic variations comprises one or more randomly integrated transposon.
95. The method of clause 94, further comprising:
c). selecting for a subsequence Saccharopolyspora strain library exhibits at least one increase in a measured phenotypic variable compared to the phenotypic performance of the base Saccharopolyspora strain.
96. The method of any one of clauses 94-95, wherein the transposon is introduced into the base Saccharopolyspora strain using a complex of transposon and transposase protein which allows for in vivo transposition of the transposon into the genome of the Saccharopolyspora strain.
97. The method of any one of clauses 94-96, wherein the transposase protein is derived from EZ-Tn5 transposome system.
98. The method of any one of clauses 94-97, wherein the transposon is a Loss-of-Function (LoF) transposon, or a Gain-of-Function (GoF) transposon.
99. The method of any one of clauses 94-98, wherein the GoF transposon comprises a solubility tag, a promoter, and/or a counter-selection marker.
100. A method for improving the phenotypic performance of a production Saccharopolyspora strain, comprising the steps of:
a. engineering the genome of a base Saccharopolyspora strain by transposon mutagenesis, to thereby create an initial transposon mutagenesis Saccharopolyspora strain library comprising a plurality of individual Saccharopolyspora strains with unique genetic variations found within each strain of said plurality of individual Saccharopolyspora strains, wherein each of said unique genetic variations comprises one or more transposon;
b. screening and selecting individual Saccharopolyspora strains of the initial transposon mutagenesis Saccharopolyspora strain library for phenotypic performance improvements over a reference Saccharopolyspora strain, thereby identifying unique genetic variations that confer phenotypic performance improvements;
c. providing a subsequent plurality of Saccharopolyspora strains that each comprise a combination of unique genetic variations from the genetic variations present in at least two individual Saccharopolyspora strains screened in the preceding step, to thereby create a subsequent transposon mutagenesis Saccharopolyspora strain library;
d. screening and selecting individual Saccharopolyspora strains of the subsequent transposon mutagenesis Saccharopolyspora strain library for phenotypic performance improvements over the reference Saccharopolyspora strain, thereby identifying unique combinations of genetic variation that confer additional phenotypic performance improvements; and
e. repeating steps c)-d) one or more times, in a linear or non-linear fashion, until a Saccharopolyspora strain exhibits a desired level of improved phenotypic performance compared to the phenotypic performance of the production Saccharopolyspora strain, wherein each subsequent iteration creates a new transposon mutagenesis Saccharopolyspora strain library of microbial strains, where each strain in the new library comprises genetic variations that are a combination of genetic variations selected from amongst at least two individual Saccharopolyspora strains of a preceding library.
101. The method for improving the phenotypic performance of a production Saccharopolyspora strain according to clause 100, wherein the subsequent transposon mutagenesis Saccharopolyspora strain library is a full combinatorial library of the initial transposon mutagenesis Saccharopolyspora strain library.
102. The method for improving the phenotypic performance of a production Saccharopolyspora strain according to any one of clauses 100-101, wherein the subsequent transposon mutagenesis Saccharopolyspora strain library is a subset of a full combinatorial library of the initial transposon mutagenesis Saccharopolyspora strain library.
103. The method for improving the phenotypic performance of a production Saccharopolyspora strain according to any one of clauses 100-102, wherein the subsequent transposon mutagenesis Saccharopolyspora strain library is a full combinatorial library of a preceding transposon mutagenesis Saccharopolyspora strain library.
104. The method for improving the phenotypic performance of a production Saccharopolyspora strain according to any one of clauses 100-103, wherein the subsequent transposon mutagenesis Saccharopolyspora strain library is a subset of a full combinatorial library of a preceding transposon mutagenesis Saccharopolyspora strain library.
105. The method for improving the phenotypic performance of a production Saccharopolyspora strain according to any one of clauses 100-104, wherein steps c)-d) are repeated until the phenotypic performance of a Saccharopolyspora strain of a subsequent transposon mutagenesis Saccharopolyspora strain library exhibits at least a 10% increase in a measured phenotypic variable compared to the phenotypic performance of the production Saccharopolyspora strain.
106. The method for improving the phenotypic performance of a production Saccharopolyspora strain according to any one of clauses 100-105, wherein steps c)-d) are repeated until the phenotypic performance of a Saccharopolyspora strain of a subsequent transposon mutagenesis Saccharopolyspora strain library exhibits at least a one-fold increase in a measured phenotypic variable compared to the phenotypic performance of the production Saccharopolyspora strain.
107. The method for improving the phenotypic performance of a production Saccharopolyspora strain according to any one of clauses 100-106, wherein the improved phenotypic performance of step e) is selected from the group consisting of: volumetric productivity of a product of interest, specific productivity of a product of interest, yield of a product of interest, titer of a product of interest, and combinations thereof.
108. The method for improving the phenotypic performance of a production Saccharopolyspora strain according to any one of clauses 100-107, wherein the improved phenotypic performance of step e) is: increased or more efficient production of a product of interest, said product of interest selected from the group consisting of: a small molecule, enzyme, peptide, amino acid, organic acid, synthetic compound, fuel, alcohol, primary extracellular metabolite, secondary extracellular metabolite, intracellular component molecule, and combinations thereof.
109. The method for improving the phenotypic performance of a production Saccharopolyspora strain according to clause 108, wherein the product of interest is selected from the group consisting of a spinosyn, spinosad, spinetoram, genistein, choline oxidase, a coumamidine compound, erythromycin, ivermectin aglycone, a HMG-CoA reductase inhibitor, a carboxylic acid isomer, alpha-methyl methionine, thialysine, alpha-ketobytarate, aspartate hydoxymate, azaserine, 5-fuoroindole, beta-hydroxynorvaline, cerulenin, purine, pyrimidine, and analogs thereof.
110. The method for improving the phenotypic performance of a production Saccharopolyspora strain according to clause 109, wherein the spinosyn is spinosyn A, spinosyn D, spinosyn J, spinosyn L, or combinations thereof.
111. The method for improving the phenotypic performance of a production Saccharopolyspora strain according to any one of clauses 100-110, wherein the transposon comprises is a Loss-of-Function (LoF) transposon, or a Gain-of-Function (GoF) transposon.
112. The method of clause 111, wherein the GoF transposon comprises a solubility tag, a promoter, and/or a counter-selection marker.

### Generating a Anti-Metabolite/Fermentation Product Resistant Saccharopolyspora Strain Library and Using the Same for Improving the Phenotypic Performance of a Production Saccharopolyspora Strain

113. A method for generating an anti-metabolite/fermentation product resistant Saccharopolyspora strain library, comprising the step of:
a) selecting for Saccharopolyspora strains resistant to a predetermined metabolite and/or a fermentation product, thereby creating an initial Saccharopolyspora strain library comprising a plurality of individual Saccharopolyspora strains with unique genetic variations found within each strain of said plurality of individual Saccharopolyspora strains, wherein at least one of said unique genetic variations results in resistance to the predetermined metabolite and/or a fermentation product; and
b) collecting Saccharopolyspora strains resistant to the predetermined metabolite and/or the fermentation product to generate the anti-metabolite/fermentation product resistant Saccharopolyspora strain library.
114. The method for generating an anti-metabolite/fermentation product resistant Saccharopolyspora strain library of clause 113, wherein the predetermined metabolite and/or fermentation product is selected from the group consisting of molecules involved in the spinosyn synthesis pathway, molecules involved in the SAM/methionine pathway, molecules involved in the lysine production pathway, molecules involved in the tryptophan pathway, molecules involved in the threonine pathway, molecules involved in the acetyl-CoA production pathway, and molecules involved in the de-novo or salvage purine and pyrimidine pathways.
115. The method for generating an anti-metabolite/fermentation product resistant Saccharopolyspora strain library of any one of clauses 113-114, wherein:
1) the molecule involved in the spinosyn synthesis pathway is a spinosyn, and optionally each strain is resistant to about 50 ug/ml to about 2 mg/ml spinosyn J/L;
2) the molecule involved in the SAM/methionine pathway is alpha-methyl methionine (aMM) or norleucine, and optionally each strain is resistant to about 1 mM to about 5 mM alpha-methyl methionine (aMM);
3) the molecule involved in the lysine production pathway is thialysine or a mixture of alpha-ketobytarate and aspartate hydoxymate;
4) the molecule involved in the tryptophan pathway is azaserine or 5-fuoroindole;
5) the molecule involved in the threonine pathway is beta-hydroxynorvaline;
6) the molecule involved in the acetyl-CoA production pathway is cerulenin, and
7) the molecule involved in the de-novo or salvage purine and pyrimidine pathways is a purine or a pyrimidine analog.
116. The method for generating an anti-metabolite/fermentation product resistant Saccharopolyspora strain library of any one of clauses 113-115, further comprising the step of:
b). selecting for a subsequence Saccharopolyspora strain library exhibits at least one increase in a measured phenotypic variable compared to the phenotypic performance of the base Saccharopolyspora strain.
117. The method for generating an anti-metabolite/fermentation product resistant Saccharopolyspora strain library of clause 116, wherein each strain in the subsequence Saccharopolyspora strain library exhibits an increased synthesis of a spinosyn.
118. A method for improving the phenotypic performance of a production Saccharopolyspora strain, comprising the steps of:
a) providing an initial anti-metabolite/fermentation product resistant Saccharopolyspora strain library comprising a plurality of individual Saccharopolyspora strains with unique genetic variations found within each strain of said plurality of individual Saccharopolyspora strains, wherein each of said unique genetic variations comprises one or more of genetic variations, wherein the genetic variations confer resistance to a predetermined metabolite or a fermentation product;
b) screening and selecting individual Saccharopolyspora strains of the initial anti-metabolite/fermentation product resistant Saccharopolyspora strain library for phenotypic performance improvements over a reference Saccharopolyspora strain, thereby identifying unique genetic variations that confer phenotypic performance improvements;
c) providing a subsequent plurality of Saccharopolyspora strains that each comprise a combination of unique genetic variations from the genetic variations present in at least two individual Saccharopolyspora strains screened in the preceding step, to thereby create a subsequent anti-metabolite/fermentation product resistant Saccharopolyspora strain library;
d) screening and selecting individual Saccharopolyspora strains of the subsequent anti-metabolite/fermentation product resistant Saccharopolyspora strain library for phenotypic performance improvements over the reference Saccharopolyspora strain, thereby identifying unique combinations of genetic variation that confer additional phenotypic performance improvements; and
e) repeating steps c)-d) one or more times, in a linear or non-linear fashion, until a Saccharopolyspora strain exhibits a desired level of improved phenotypic performance compared to the phenotypic performance of the production Saccharopolyspora strain, wherein each subsequent iteration creates a new anti-metabolite/fermentation product resistant Saccharopolyspora strain library of microbial strains, where each strain in the new library comprises genetic variations that are a combination of genetic variations selected from amongst at least two individual Saccharopolyspora strains of a preceding library.
119. The method for improving the phenotypic performance of a production Saccharopolyspora strain according to clause 118, wherein the subsequent anti-metabolite/fermentation product resistant Saccharopolyspora strain library is a full combinatorial library of the initial anti-metabolite/fermentation product resistant Saccharopolyspora strain library.
120. The method for improving the phenotypic performance of a production Saccharopolyspora strain according to any one of clauses 118-119, wherein the subsequent anti-metabolite/fermentation product resistant Saccharopolyspora strain library is a subset of a full combinatorial library of the initial anti-metabolite/fermentation product resistant Saccharopolyspora strain library.
121. The method for improving the phenotypic performance of a production Saccharopolyspora strain according to any one of clauses 118-120, wherein the subsequent anti-metabolite/fermentation product resistant Saccharopolyspora strain library is a full combinatorial library of a preceding anti-metabolite/fermentation product resistant Saccharopolyspora strain library.
122. The method for improving the phenotypic performance of a production Saccharopolyspora strain according to any one of clauses 118-122, wherein the subsequent anti-metabolite/fermentation product resistant Saccharopolyspora strain library is a subset of a full combinatorial library of a preceding anti-metabolite/fermentation product resistant Saccharopolyspora strain library.
123. The method for improving the phenotypic performance of a production Saccharopolyspora strain according to any one of clauses 118-122, wherein steps c)-d) are repeated until the phenotypic performance of a Saccharopolyspora strain of a subsequent anti-metabolite/fermentation product resistant Saccharopolyspora strain library exhibits at least a 10% increase in a measured phenotypic variable compared to the phenotypic performance of the production Saccharopolyspora strain.
124. The method for improving the phenotypic performance of a production Saccharopolyspora strain according to any one of clauses 118-123, wherein steps c)-d) are repeated until the phenotypic performance of a Saccharopolyspora strain of a subsequent anti-metabolite/fermentation product resistant Saccharopolyspora strain library exhibits at least a one-fold increase in a measured phenotypic variable compared to the phenotypic performance of the production Saccharopolyspora strain.
125. The method for improving the phenotypic performance of a production Saccharopolyspora strain according to any one of clauses 118-124, wherein the improved phenotypic performance of step e) is selected from the group consisting of: volumetric productivity of a product of interest, specific productivity of a product of interest, yield of a product of interest, titer of a product of interest, and combinations thereof.
126. The method for improving the phenotypic performance of a production Saccharopolyspora strain according to clause 125, wherein the improved phenotypic performance of step e) is: increased or more efficient production of a product of interest, said product of interest selected from the group consisting of: a small molecule, enzyme, peptide, amino acid, organic acid, synthetic compound, fuel, alcohol, primary extracellular metabolite, secondary extracellular metabolite, intracellular component molecule, and combinations thereof.
127. The method for improving the phenotypic performance of a production Saccharopolyspora strain according to clause 126, wherein the product of interest is selected from the group consisting of a spinosyn, spinosad, spinetoram, genistein, choline oxidase, a coumamidine compound, erythromycin, ivermectin aglycone, a HMG-CoA reductase inhibitor, a carboxylic acid isomer, alpha-methyl methionine, thialysine, alpha-ketobytarate, aspartate hydoxymate, azaserine, 5-fuoroindole, beta-hydroxynorvaline, cerulenin, purine, pyrimidine, and analogs thereof.
128. The method for improving the phenotypic performance of a production Saccharopolyspora strain according to clause 127, wherein the spinosyn is spinosyn A, spinosyn D, spinosyn J, spinosyn L, or combinations thereof.

### Saccharopolyspora Host Cells and Strain Libraries

129. A Saccharopolyspora host cell comprising a promoter operably linked to an endogenous gene of the host cell, wherein the promoter is heterologous to the endogenous gene, wherein the promoter has a sequence selected from the group consisting of SEQ ID Nos. 1-69.
130. The Saccharopolyspora host cell of clause 129, wherein the endogenous gene is involved in synthesis of a spinosyn in the Saccharopolyspora host cell.
131. The Saccharopolyspora host cell of any one of clauses 129-130, wherein Saccharopolyspora host cell has a desired level of improved phenotypic performance compared to the phenotypic performance of a reference Saccharopolyspora strain without the promoter operably linked to the endogenous gene.
132. A Saccharopolyspora strain library, wherein each Saccharopolyspora strain in the library comprises a promoter operably linked to an endogenous gene of the host cell, wherein the promoter is heterologous to the endogenous gene, wherein the promoter has a sequence selected from the group consisting of SEQ ID Nos. 1-69.
133. A Saccharopolyspora host cell comprising a terminator linked to an endogenous gene of the host cell, wherein the terminator is heterologous to the endogenous gene, wherein the promoter has a sequence selected from the group consisting of SEQ ID Nos. 70-80.
134. The Saccharopolyspora host cell of clause 133, wherein the endogenous gene is involved in synthesis of a spinosyn in the Saccharopolyspora host cell.
135. The Saccharopolyspora host cell of any one of clauses 133-134, wherein Saccharopolyspora host cell has a desired level of improved phenotypic performance compared to the phenotypic performance of a reference Saccharopolyspora strain without the promoter operably linked to the endogenous gene.
136. A Saccharopolyspora strain library, wherein each Saccharopolyspora strain in the library comprises a terminator linked to an endogenous gene of the host cell, wherein the terminator is heterologous to the endogenous gene, wherein the terminator has a sequence selected from the group consisting of SEQ ID Nos. 70-80.
137. A Saccharopolyspora host cell comprising a ribosomal binding site operably linked to an endogenous gene of the host cell, wherein the ribosomal binding site is heterologous to the endogenous gene, wherein the ribosomal binding site has a sequence selected from the group consisting of SEQ ID Nos. 97-127.
138. The Saccharopolyspora host cell of clause 137, wherein the endogenous gene is involved in synthesis of a spinosyn in the Saccharopolyspora host cell.
139. The Saccharopolyspora host cell of any one of clauses 137-138, wherein Saccharopolyspora host cell has a desired level of improved phenotypic performance compared to the phenotypic performance of a reference Saccharopolyspora strain without the RBS operably linked to the endogenous gene.
140. A Saccharopolyspora strain library, wherein each Saccharopolyspora strain in the library comprises a ribosomal binding site operably linked to an endogenous gene of the host cell, wherein the ribosomal binding site is heterologous to the endogenous gene, wherein the ribosomal binding site has a sequence selected from the group consisting of SEQ ID Nos. 97-127.
141. A Saccharopolyspora host cell comprising a transposon, wherein Saccharopolyspora host cell has a desired level of improved phenotypic performance compared to the phenotypic performance of a reference Saccharopolyspora strain without the transposon.
142. The Saccharopolyspora host cell of clause 141, wherein the transposon is a Loss-of-Function (LoF) transposon, or a Gain-of-Function (GoF) transposon.
143. The Saccharopolyspora host cell of clause 142, wherein the Gain-of-Function (GoF) transposon comprises a promoter, a counterselection marker, and/or a solubility tag.
144. The Saccharopolyspora host cell of any one of clauses 141-143, wherein the transposon comprises a sequence selected from the group consisting of SEQ ID No. 128-131.
145. A Saccharopolyspora strain library, wherein each Saccharopolyspora strain in the library comprises a transposon having a sequence selected from the group consisting of SEQ ID No. 128-131, wherein the transposon in each strain is at a different genomic locus.
146. A Saccharopolyspora strain library, wherein each Saccharopolyspora strain in the library comprises a genetic variation that results in resistance of the strain to
1) a molecule involved in the spinosyn synthesis pathway,
2) a molecule involved in the SAM/methionine pathway,
3) a molecule involved in the lysine production pathway,
4) a molecule involved in the tryptophan pathway,
5) a molecule involved in the threonine pathway,
6) a molecule involved in the acetyl-CoA production pathway, and/or
7) a molecule involved in the de-novo or salvage purine and pyrimidine pathways.
147. The Saccharopolyspora strain library of clause 146, wherein:
1) the molecule involved in the spinosyn synthesis pathway is a spinosyn;
2) the molecule involved in the SAM/methionine pathway is alpha-methyl methionine (aMM) or norleucine;
3) the molecule involved in the lysine production pathway is thialysine or a mixture of alpha-ketobytarate and aspartate hydoxymate;
4) the molecule involved in the tryptophan pathway is azaserine or 5-fuoroindole;
5) the molecule involved in the threonine pathway is beta-hydroxynorvaline;
6) the molecule involved in the acetyl-CoA production pathway is cerulenin; and
7) the molecule involved in the de-novo or salvage purine and pyrimidine pathways is a purine or a pyrimidine analog.
148. The Saccharopolyspora strain library of clause 147, wherein the molecule is spinosyn J/L, and wherein each strain is resistant to about 50 ug/ml to about 2 mg/ml spinosyn J/L.
149. The Saccharopolyspora strain library of clause 147, wherein the molecule is alpha-methyl methionine (aMM), wherein each strain is resistant to about 1 mM to about 5 mM aMM.
150. A Saccharopolyspora strain comprising a reporter gene, wherein the reporter gene is selected from the group consisting of:
a) genes encoding a green fluorescent reporter protein, optionally the genes are codon optimized for expression in Saccharopolyspora;
b) genes encoding a green fluorescent reporter protein, optionally the genes are codon optimized for expression in Saccharopolyspora; and
c) genes encoding a beta-glucuronidase (gusA) protein, optionally the genes are codon optimized for expression in Saccharopolyspora.
151. The Saccharopolyspora strain of clause 150, wherein:
a) the green fluorescent reporter protein has the amino acid sequence of SEQ ID No. 143;
b) the red fluorescent reporter protein has the amino acid sequence of SEQ ID No. 144; and
c) the gusA protein has the amino acid sequence of SEQ ID No. 145.
152. The Saccharopolyspora strain of clause 150, wherein:
a) the gene encoding the green fluorescent reporter protein has the sequence of SEQ ID No. 81;
b) the gene encoding the red fluorescent reporter protein has the sequence of SEQ ID No. 82; and
c) the gene encoding the gusA protein has sequence of SEQ ID No. 83.
153. The Saccharopolyspora strain of any one of clauses 150-153, wherein the strain comprises both the gene encoding the green fluorescent reporter protein, and the gene encoding the red fluorescent reporter protein, wherein the fluorescent excitation and emission spectra of the green fluorescent reporter protein and the red fluorescent reporter protein are distinct from each other.
154. The Saccharopolyspora strain of any one of clauses 150-153, wherein the strain comprises both the gene encoding the green fluorescent reporter protein, and the gene encoding the red fluorescent reporter protein, wherein the fluorescent excitation and emission spectra of the green fluorescent reporter protein and the red fluorescent reporter protein are distinct from the endogenous fluorescence of the Saccharopolyspora strain.
155. A Saccharopolyspora strain comprising a DNA fragment integrated into one or more neutral integration sites in the genome of the Saccharopolyspora strain, wherein the neutral integration sites are selected from the group of positions within a genomic fragment having a sequence selected from SEQ ID Nos. 132-142, or genomic fragments homologous to any one of SEQ ID Nos. 132-142.
156. The Saccharopolyspora strain of clause 155, wherein the Saccharopolyspora strain has a desired level of improved phenotypic performance compared to the phenotypic performance of a reference Saccharopolyspora strain without the integrated DNA fragment.
157. The Saccharopolyspora strain of clause 156, wherein the Saccharopolyspora strain has a desired level of improved spinosyn production compared to the phenotypic performance of a reference Saccharopolyspora strain without the integrated DNA fragment.
158. The Saccharopolyspora strain of any one of clauses 155-157, wherein the integrated DNA fragment comprises a sequence encoding for a reporter protein.
159. The Saccharopolyspora strain of any one of clauses 155-158, wherein the integrated DNA fragment comprises a transposon.
160. The Saccharopolyspora strain of any one of clauses 155-159, wherein the integrated DNA fragment comprises an attachment site (attB) which can be recognized by its corresponding integrase.

### Neutral Integration Sites (NISs) for Integrating DNA Fragment in Saccharopolyspora Strain

161. A method of integrating a DNA fragment into the genome of a Saccharopolyspora strain, wherein the DNA fragment is integrated into a neutral integration site in the genome of the Saccharopolyspora strain, wherein the neutral integration site is selected from the group of positions within a genomic fragment having a sequence selected from SEQ ID Nos. 132-142, or genomic fragments homologous to any one of SEQ ID Nos. 132-142.
162. The method of integrating a DNA fragment into the genome of a Saccharopolyspora strain of clause 161, wherein the DNA fragment comprises an attachment site (attB) which can be recognized by its corresponding integrase.
163. A method for rapidly consolidating genetic mutations derived from at least two parental Saccharopolyspora strains, comprising the steps of:
(1) providing at least two parental Saccharopolyspora strains, wherein each strain comprises a unique genomic mutation that does not exist in the other strains;
(2) preparing protoplasts from each of the parental strains;
(3) fusing the protoplasts from the parental strains to produce fused protoplast comprising the genomes of two parental Saccharopolyspora strains, wherein homologous recombination between the genomes of each parental strain occurs;
(4) recovering Saccharopolyspora cells from the fused protoplast produced in step (3); and
(5) selecting for Saccharopolyspora cells comprising the unique genomic mutation of a first parental Saccharopolyspora strain; and
(6) genotyping the Saccharopolyspora cells obtained in step (5) for the presence of the unique genomic mutation of a second parental strain,
thereby obtaining a new Saccharopolyspora strain comprising the unique genomic mutations derived from two parental Saccharopolyspora strains.
164. The method of clause 163, wherein one of the unique genomic mutations is linked to a selectable marker, while the other unique genomic mutation is not linked to any selectable marker.
165. The method of clause 164, wherein in step (3) the ratio of protoplasts of the stain originally containing the unique genomic mutation linked to the selectable marker:protoplasts of the stain originally containing the unique genomic mutation not linked to the selectable marker is less than 1:1.
166. The method of clause 165, wherein the ratio is about 1:10 to about 1:100, or less.
167. The method of any one of clauses 163-166, wherein in step (4), protoplast cells are plated on an osmotically stabilized media without the use of agar overlay.
168. The method of any one of clauses 163-167, wherein step (5) is accomplished by overlaying an appropriate selection drug antibiotic onto the growing cells, when one of the unique genomic mutations is linked to a selectable marker which results in resistance to the selection drug.
169. The method of any one of clauses 163-168, wherein step (5) is accomplished by genotyping, when none of the unique genomic mutations is linked to a selectable marker.
170. The method of any one of clauses 163-170, wherein genetic mutations derived from more than two strains are randomly consolidated during a single consolidation process.
171. The method of any one of clauses 163-171, wherein in step (2) the protoplasts are initially collected by centrifuging at a speed about 5000×g for about 5 minutes.
172. The method of any one of clauses 163-172, wherein the method does not comprise of filtrating the protoplasts through cotton wool.
173. The method of any one of clauses 163-173, wherein the fused protoplasts are recovered on a R2YE media rather than top-agar.
174. The method of clause 173, wherein the R2YE media comprises 0.5M sorbitol and 0.5M mannose.

### Targeted Genome Editing in Saccharopolyspora Strains

175. A method of targeted genome editing in a Saccharopolyspora strain, comprising:
a) introducing a plasmid comprising a selection marker, a counterselection marker, a DNA fragment having homology to the genomic locus of the Saccharopolyspora strain to be edited, and plasmid backbone sequence into a base Saccharopolyspora strain;
b) selecting for Saccharopolyspora strains with integration event based on the presence of the selection marker in the genome;
c) selecting for Saccharopolyspora strains having the plasmid backbone looped out based on the absence of the counterselection marker gene, wherein the counterselection marker is a sacB gene or a pheS gene.
176. The method of clause 175, wherein the resulted Saccharopolyspora strain with edited genome has better performance compared to the parent strain without the editing.
177. The method of clause 176, wherein the resulted Saccharopolyspora strain has increased spinosyn production compared to the parent strain without the editing.
178. The method of any one of clauses 175-177, wherein the sacB gene is codon-optimized for Saccharopolyspora spinosa.
179. The method of clause 178, wherein the sacB gene encodes an amino acid sequence with 90% sequence identity to the amino acid sequence encoded by SEQ ID No. 146.
180. The method of any one of clauses 175-177, wherein the pheS gene is codon-optimized for Saccharopolyspora spinosa.
181. The method of clause 180, wherein the pheS gene encodes an amino acid sequence with 90% sequence identity to the amino acid encoded by SEQ ID No. 147 or SEQ ID No. 148.
Transferring Genetic Material from Donor Microorganism Cells to Recipient Cells of a Saccharopolyspora Microorganism Using Conjugation
182. A method of transferring genetic material from donor microorganism cells to recipient cells of a Saccharopolyspora microorganism, wherein the method comprises the steps of:
1) Optionally, subculturing recipient cells to late-exponential or stationary phase;
2) Optionally, subculturing donor cells to mid-exponential phase;
3) Combining donor and recipient cells;
4) Plating donor and recipient cell mixture on conjugation media;
5) Incubating plates to allow cells to conjugate;
6) Applying antibiotic selection against donor cells;
7) Applying antibiotic selection against non-integrated recipient cells; and
8) further incubating plates to allow for the outgrowth of integrated recipient cells.
   183. The method of clause 182, wherein the donor microorganism cells are E. coli cells.
   184. The method of any one of clauses 182-183, wherein at least two, three, four, five, six, seven or more of the following conditions are utilized:
1) recipient cells are washed before conjugating;
2) donor cells and recipient cells are conjugated at a temperature of about 30° C.;
3) recipient cells are sub-cultured for at least about 48 hours before conjugating;
4) the ratio of donor cells:recipient cells for conjugation is about 1:0.6 to 1:1.0;
5) an antibiotic drug for selection against the donor cells is delivered to the mixture about 15 to 24 hours after the donor cells and the recipient cells are mixed;
6) an antibiotic drug for selection against the recipient cells is delivered to the mixture about 40 to 48 hours after the donor cells and the recipient cells are mixed;
7) the conjugation media plated with donor and recipient cell mixture is dried for at least about 3 hours to 10 hours;
8) the conjugation media comprises at least about 3 g/L glucose;
9) the concentration of donor cells is about OD600=0.1 to 0.6;
10) the concentration of recipient cells is about OD540=5.0 to 15.0;
   185. The method of clause 184, wherein the antibiotic drug for selection against the donor cells is nalidixic, and the concentration is about 50 to about 150 μg/ml.
   186. The method of clause 185, wherein the antibiotic drug for selection against the donor cells is nalidixic, and the concentration is about 100 μg/ml.
   187. The method of clause 184, wherein the antibiotic drug for selection against the recipient cells is apramycin, and the concentration is about 50 to about 250 μg/ml.
   188. The method of clause 187, wherein the antibiotic drug for selection against the recipient cells is apramycin, and the concentration is about 100 μg/ml.
   189. The method of any one of clauses 182-188, wherein the method is performed in a high-throughput process.
   190. The method of clause 189, wherein the method is performed on a 48-well Q-trays.
   191. The method of clause 189, wherein the high-throughput process is automated.
   192. The method of clause 191, where the mixture of donor cells and recipient cells is a liquid mixture, and ample volume of the liquid mixture is plated on the medium with a rocking motion, wherein the liquid mixture is dispersed over the whole area of the medium.
   193. The method of clause 191, wherein the method comprises automated process of transferring exconjugants by colony picking with yeast pins for subsequent inoculation of recipient cells with integrated DNA provided by the donor cells.
   194. The method of clause 193, the colony picking is performed in either a dipping motion, or a stirring motion.
   195. The method of any one of clauses 184-194, wherein the conjugating media is a modified ISP4 media comprising about 3-10 g/L glucose.
   196. The method of any one of clauses 184-194, wherein the total number of donor cells or recipient cells in the mixture is about 5×10⁶ to about 9×10⁶.
   197. The method of any one of clauses 182-196, wherein the method is performed with at least four of the following conditions:
1) recipient cells are washed before conjugating;
2) donor cells and recipient cells are conjugated at a temperature of about 30° C.;
3) recipient cells are sub-cultured for at least about 48 hours before conjugating;
4) the ratio of donor cells:recipient cells for conjugation is about 1:0.8;
5) an antibiotic drug for selection against the donor cells is delivered to the mixture about 20 hours after the donor cells and the recipient cells are mixed;
6) the amount of the donor cells or the amount of the recipient cells in the mixture is about 7×10⁶, and
7) the conjugation media comprises about 6 g/L glucose.

### Scarless Method of Targeted Genomic Editing in a Saccharopolyspora Strain

198. A method of targeted genomic editing in a Saccharopolyspora strain, resulting in a scarless Saccharopolyspora strain containing a genetic variation at a targeted genomic locus, comprising:
a) introducing a plasmid into a Saccharopolyspora strain, said plasmid comprising:
   i. a selection marker,
   ii. a counterselection marker,
   iii. a DNA fragment containing a genetic variation to be integrated into the Saccharopolyspora genome at a target locus, said DNA fragment having homology arms to the target genomic locus flanking the desired genetic variation, and
   iv. plasmid backbone sequence;
b) selecting for a Saccharopolyspora strain that has undergone an initial homologous recombination and has the genetic variation integrated into the target locus based on the presence of the selection marker in the genome; and
c) selecting for a Saccharopolyspora strain that has the genetic variation integrated into the target locus, but has undergone an additional homologous recombination that loops-out the plasmid backbone, based on the absence of the counterselection marker,
wherein said targeted genomic locus may comprise any region of the Saccharopolyspora genome, including genomic regions that do not contain repeating segments of encoding DNA modules.
   199. The method of clause 198, wherein the plasmid does not comprise a temperature sensitive replicon.
   200. The method of any one of clauses 198-199, wherein the plasmid does not comprise an origin of replication.
   201. The method of any one of clauses 198-200, wherein the selection step (c) is performed without replication of the integrated plasmid.
   202. The method of any one of clauses 198-201, wherein the plasmid is a single homologous recombination vector.
   203. The method of any one of clauses 198-202, wherein the plasmid is a double homologous recombination vector.
   204. The method of any one of clauses 198-203, wherein the counterselection marker is a sacB gene or a pheS gene.
   205. The method of clause 204, wherein the sacB gene or pheS gene is codon-optimized for Saccharopolyspora spinosa.
   206. The method of clause 205, wherein the sacB gene encodes an amino acid sequence with 90% sequence identity to the amino acid sequence encoded by SEQ ID NO. 146.
   207. The method of clause 205, wherein the pheS gene encodes an amino acid sequence with 90% sequence identity to the amino acid encoded by SEQ ID NO. 147 or SEQ ID NO. 148.
   208. The method of any one of clauses 198-207, wherein the plasmid is introduced into the Saccharopolyspora strain by transformation.
   209. The method of any one of clauses 198-208, wherein the transformation is a protoplast transformation.
   210. The method of any one of clauses 198-209, wherein the plasmid is introduced into the Saccharopolyspora strain by conjugation, wherein the Saccharopolyspora strain is a recipient cell, and a donor cell comprising the plasmid transfers the plasmid to the Saccharopolyspora strain.
   211. The method of any one of clauses 198-210, wherein the conjugation is based on an E. coli donor cell comprising the plasmid.
   212. The method of any one of clauses 198-211, wherein the target locus is a locus associated with production of a compound of interest in the Saccharopolyspora strain.
   213. The method of any one of clauses 198-212, wherein the resulting Saccharopolyspora strain has increased production of a compound of interest compared to a control strain without the genomic editing.
   214. The method of clause 212 or 213, wherein the compound of interest is a spinosyn.
   215. The method of any one of clauses 198-214, wherein the method is performed as a high-throughput procedure.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

In addition, International Application No. PCT/US2016/065464, filed on Dec. 7, 2016, which claims the benefit of priority to U.S. Provisional Application No. 62/264,232, filed on Dec. 7, 2015, U.S. Nonprovisional application Ser. No. 15/140,296, filed on Apr. 27, 2016, International Application No. PCT/US2017/29725, filed Apr. 27, 2017, U.S. Nonprovisional application Ser. No. 15/396,230, filed on Dec. 30, 2016, and U.S. Provisional Application No. 62/368,786, filed on Jul. 29, 2016, are all hereby incorporated by reference in their entirety, including all descriptions, references, figures, and claims for all purposes.

What is claimed is:

1. A high-throughput (HTP) method of genomic engineering to evolve a Saccharopolyspora sp. microbe to acquire a desired phenotype, comprising:
a. perturbing the genomes of an initial plurality of Saccharopolyspora microbes having the same genomic strain background, to thereby create an initial HTP genetic design Saccharopolyspora strain library comprising individual Saccharopolyspora strains with unique genetic variations;
b. screening and selecting individual Saccharopolyspora strains of the initial HTP genetic design Saccharopolyspora strain library for the desired phenotype;
c. providing a subsequent plurality of Saccharopolyspora microbes that each comprise a unique combination of genetic variation, said genetic variation selected from the genetic variation present in at least two individual Saccharopolyspora strains screened in the preceding step, to thereby create a subsequent HTP genetic design Saccharopolyspora strain library;
d. screening and selecting individual Saccharopolyspora strains of the subsequent HTP genetic design Saccharopolyspora strain library for the desired phenotype; and
e. repeating steps c)-d) one or more times, in a linear or non-linear fashion, until a Saccharopolyspora microbe has acquired the desired phenotype, wherein each subsequent iteration creates a new HTP genetic design Saccharopolyspora strain library comprising individual Saccharopolyspora strains harboring unique genetic variations that are a combination of genetic variation selected from amongst at least two individual Saccharopolyspora strains of a preceding HTP genetic design Saccharopolyspora strain library.

2. The HTP method of genomic engineering according to claim 1, wherein the function and/or identity of the genes that contain the genetic variations are not considered before the genetic variations are combined in step (b).

3. The HTP method of genomic engineering according to claim 1, wherein at least one genetic variation to be combined is not in a genomic region that contains repeating segments of encoding DNA modules.

4. The HTP method of genomic engineering according to claim 1, wherein the subsequent plurality of Saccharopolyspora microbes that each comprises a unique combination of genetic variations in step (c) are produced by:
1) introducing a plasmid into an individual Saccharopolyspora strain belonging to the initial HTP genetic design Saccharopolyspora strain library, wherein the plasmid comprises a selection marker, a counterselection marker, a DNA fragment having homology to the genomic locus of the base Saccharopolyspora strain, and plasmid backbone sequence, wherein the DNA fragment has a genetic variation derived from another individual Saccharopolyspora strain also belonging to the initial HTP genetic design Saccharopolyspora strain library;
2) selecting for Saccharopolyspora strains with integration event based on the presence of the selection marker in the genome;
3) selecting for Saccharopolyspora strains having the plasmid backbone looped out based on the absence of the counterselection marker gene.

5. The HTP method of claim 4, wherein the plasmid does not comprise a temperature sensitive replicon.

6. The HTP method of claim 4, wherein the selection step (3) is performed without replication of the integrated plasmid.

7. The HTP method of genomic engineering according to claim 1, wherein the initial HTP genetic design Saccharopolyspora strain library comprises at least one library selected from the group consisting of a promoter swap microbial strain library, SNP swap microbial strain library, start/stop codon microbial strain library, optimized sequence microbial strain library, a terminator swap microbial strain library, a transposon mutagenesis microbial strain diversity library, a ribosomal binding site microbial strain library, an anti-metabolite/fermentation product resistance library, a termination insertion microbial strain library, and any combination thereof.

8. The HTP method of genomic engineering according to claim 1, wherein the subsequent HTP genetic design Saccharopolyspora strain library is a full combinatorial Saccharopolyspora strain library of the initial HTP genetic design microbial strain library.

9. The HTP method of genomic engineering according to claim 1, wherein the subsequent HTP genetic design Saccharopolyspora strain library is a subset of a full combinatorial Saccharopolyspora strain library derived from the genetic variations in the initial HTP genetic design Saccharopolyspora strain library.

10. The HTP method of genomic engineering according to claim 1, wherein the subsequent HTP genetic design derived from the genetic variations in strain library is a full combinatorial microbial strain library derived from the genetic variations in a preceding HTP genetic design Saccharopolyspora strain library.

11. The HTP method of genomic engineering according to claim 1, wherein the subsequent HTP genetic design Saccharopolyspora strain library is a subset of a full combinatorial Saccharopolyspora strain library derived from the genetic variations in a preceding HTP genetic design Saccharopolyspora strain library.

12. The HTP method of genomic engineering according to claim 1, wherein perturbing the genome comprises utilizing at least one method selected from the group consisting of: random mutagenesis, targeted sequence insertions, targeted sequence deletions, targeted sequence replacements, transposon mutagenesis, and any combination thereof.

13. The HTP method of genomic engineering according to claim 1, wherein the initial plurality of Saccharopolyspora microbes comprise unique genetic variations derived from a production Saccharopolyspora strain.

14. The HTP method of genomic engineering according to claim 1, wherein the initial plurality of Saccharopolyspora microbes comprise production strain microbes denoted S₁Gen₁ and any number of subsequent microbial generations derived therefrom denoted SₙGenₙ.

15. The HTP method of genomic engineering according to claim 1, wherein the step c comprises rapidly consolidating the genetic variations by using protoplast fusion techniques.

16. The HTP method of genomic engineering according to claim 1, wherein the initial HTP genetic design Saccharopolyspora strain library or the subsequent HTP genetic design Saccharopolyspora strain library comprises a promoter swap microbial strain library.

17. The HTP method of genomic engineering according to claim 16, wherein the promoter swap microbial strain library comprises at least one promoter with a nucleotide sequence selected from SEQ ID Nos. 1 to 69 and 172 to 175.

18. The HTP method of genomic engineering according to claim 1, wherein the initial HTP genetic design Saccharopolyspora strain library or the subsequent HTP genetic design Saccharopolyspora strain library comprises a SNP swap microbial strain library.

19. The HTP method of genomic engineering according to claim 1, wherein the initial HTP genetic design Saccharopolyspora strain library or the subsequent HTP genetic design Saccharopolyspora strain library comprises a terminator swap microbial strain library.

20. The HTP method of genomic engineering according to claim 19, wherein the terminator swap microbial strain library comprises at least one terminator with a nucleotide sequence selected from SEQ ID Nos. 70 to 80.

21. The HTP method of genomic engineering according to claim 1, wherein the initial HTP genetic design Saccharopolyspora strain library or the subsequent HTP genetic design Saccharopolyspora strain library comprises a transposon mutagenesis microbial strain diversity library.

22. The HTP method of genomic engineering according to claim 21, wherein the initial HTP genetic design Saccharopolyspora strain library or the subsequent HTP genetic design Saccharopolyspora strain library comprises a Loss-of-Function (LoF) transposon and/or a Gain-of-Function (GoF) transposon.

23. The HTP method of genomic engineering according to claim 22, wherein the GoF transposon comprises a solubility tag, a promoter, and/or a counter-selection marker.

24. The HTP method of genomic engineering according to claim 1, wherein the initial HTP genetic design Saccharopolyspora strain library or the subsequent HTP genetic design Saccharopolyspora strain library comprises a ribosomal binding site microbial strain library.

25. The HTP method of genomic engineering according to claim 24, wherein ribosomal binding site microbial strain library comprises at least one ribosomal binding site (RBS) with a nucleotide sequence selected from SEQ ID Nos. 97 to 127.

26. The HTP method of genomic engineering according to claim 1, wherein the initial HTP genetic design Saccharopolyspora strain library or the subsequent HTP genetic design Saccharopolyspora strain library comprises an anti-metabolite/fermentation product resistance library.

27. The HTP method of genomic engineering according to claim 26, wherein the anti-metabolite/fermentation product resistance library comprises a Saccharopolyspora strain resistance to a molecule involved in spinosyn synthesis in Saccharopolyspora.

28. A method for generating a SNP swap Saccharopolyspora strain library, comprising the steps of:
a. providing a reference Saccharopolyspora strain and a second Saccharopolyspora strain, wherein the second Saccharopolyspora strain comprises a plurality of identified genetic variations selected from single nucleotide polymorphisms, DNA insertions, and DNA deletions, which are not present in the reference Saccharopolyspora strain; and
b. perturbing the genome of either the reference Saccharopolyspora strain, or the second Saccharopolyspora strain, to thereby create an initial SNP swap Saccharopolyspora strain library comprising a plurality of individual Saccharopolyspora strains with unique genetic variations found within each strain of said plurality of individual Saccharopolyspora strains, wherein each of said unique genetic variations corresponds to a single genetic variation selected from the plurality of identified genetic variations between the reference Saccharopolyspora strain and the second Saccharopolyspora strain.

29. The method for generating a SNP swap Saccharopolyspora strain library according to claim 28, wherein the genome of the reference Saccharopolyspora strain is perturbed to add one or more of the identified single nucleotide polymorphisms, DNA insertions, or DNA deletions, which are found in the second Saccharopolyspora strain.

30. The method for generating a SNP swap Saccharopolyspora strain library according to claim 28, wherein the genome of the second Saccharopolyspora strain is perturbed to remove one or more of the identified single nucleotide polymorphisms, DNA insertions, or DNA deletions, which are not found in the reference Saccharopolyspora strain.

31. The method for generating a SNP swap Saccharopolyspora strain library according to claim 28, wherein the resultant plurality of individual Saccharopolyspora strains with unique genetic variations, together comprise a full combinatorial library of all the identified genetic variations between the reference Saccharopolyspora strain and the second Saccharopolyspora strain.

32. The method for generating a SNP swap Saccharopolyspora strain library according to claim 28, wherein the resultant plurality of individual Saccharopolyspora strains with unique genetic variations, together comprise a subset of a full combinatorial library of all the identified genetic variations between the reference Saccharopolyspora strain and the second Saccharopolyspora strain.

33. A method for rehabilitating and improving the phenotypic performance of a production Saccharopolyspora strain, comprising the steps of:
a. providing a parental lineage Saccharopolyspora strain and a production Saccharopolyspora strain derived therefrom, wherein the production Saccharopolyspora strain comprises a plurality of identified genetic variations selected from single nucleotide polymorphisms, DNA insertions, and DNA deletions, not present in the parental lineage Saccharopolyspora strain;
b. perturbing the genome of either the parental lineage Saccharopolyspora strain, or the production Saccharopolyspora strain, to thereby create an initial Saccharopolyspora strain library. Wherein each strain in the initial library comprises a unique genetic variation from the plurality of identified genetic variations between the parental lineage Saccharopolyspora strain and the production Saccharopolyspora strain;
c. screening and selecting individual Saccharopolyspora strains of the initial SNP swap Saccharopolyspora strain library for phenotype performance improvements over a reference Saccharopolyspora strain, thereby identifying unique genetic variations that confer phenotypic performance improvements;
d. providing a subsequent plurality of microbes that each comprise a combination of unique genetic variation from the variations present in at least two individual Saccharopolyspora strains screened in the preceding step, to thereby create a subsequent library of Saccharopolyspora strains;
e. screening and selecting individual strains of the subsequent strain library for phenotypic performance improvements over the reference Saccharopolyspora strain, thereby identifying unique combinations of genetic variation that confer additional phenotypic performance improvements; and
f. repeating steps d)-e) one or more times, in a linear or non-linear fashion, until a Saccharopolyspora strain exhibits a desired level of improved phenotypic performance compared to the phenotypic performance of the production Saccharopolyspora strain, wherein each subsequent iteration creates a new library of Saccharopolyspora strains, where each strain in the new library comprises genetic variations that are a combination of genetic variations selected from amongst at least two individual Saccharopolyspora strains of a preceding library.

34. The method for rehabilitating and improving the phenotypic performance of a production Saccharopolyspora strain according to claim 33, wherein the initial library of Saccharopolyspora strains is a full combinatorial library comprising all of the identified genetic variations between the parental lineage Saccharopolyspora strain and the production Saccharopolyspora strain.

35. The method for rehabilitating and improving the phenotypic performance of a production Saccharopolyspora strain according to claim 33, wherein the initial library of Saccharopolyspora strains is a subset of a full combinatorial library comprising a subset of the identified genetic variations between the reference parental lineage Saccharopolyspora strain and the production Saccharopolyspora strain.

36. The method for rehabilitating and improving the phenotypic performance of a production Saccharopolyspora strain according to claim 33, wherein the subsequent library of Saccharopolyspora strains is a full combinatorial library of the initial library.

37. The method for rehabilitating and improving the phenotypic performance of a production Saccharopolyspora strain according to claim 33, wherein the subsequent library of Saccharopolyspora strains is a full combinatorial library of the initial library.

38. The method for rehabilitating and improving the phenotypic performance of a production Saccharopolyspora strain according to claim 33, wherein the subsequent library of Saccharopolyspora strains is a full combinatorial library of a preceding library.

39. The method for rehabilitating and improving the phenotypic performance of a production Saccharopolyspora strain according to claim 33, wherein the subsequent library of Saccharopolyspora strains is a subset of a full combinatorial library of a preceding library.

40. The method for rehabilitating and improving the phenotypic performance of a production Saccharopolyspora strain according to claim 33, wherein the genome of the parental lineage Saccharopolyspora strain is perturbed to add one or more of the identified single nucleotide polymorphisms, DNA insertions, or DNA deletions, which are found in the production Saccharopolyspora strain.

41. The method for rehabilitating and improving the phenotypic performance of a production Saccharopolyspora strain according to claim 33, wherein the genome of the production Saccharopolyspora strain is perturbed to remove one or more of the identified single nucleotide polymorphisms, DNA insertions, or DNA deletions, which are not found in the parental lineage Saccharopolyspora strain.

42. The method for rehabilitating and improving the phenotypic performance of a production Saccharopolyspora strain according to claim 33, wherein perturbing the genome comprises utilizing at least one method selected from the group consisting of: random mutagenesis, targeted sequence insertions, targeted sequence deletions, targeted sequence replacements, and combinations thereof.

43. The method for rehabilitating and improving the phenotypic performance of a production Saccharopolyspora strain according to claim 33, wherein steps d)-e) are repeated until the phenotypic performance of a Saccharopolyspora strain of a subsequent library exhibits at least a 10% increase in a measured phenotypic variable compared to the phenotypic performance of the production Saccharopolyspora strain.

44. The method for rehabilitating and improving the phenotypic performance of a production Saccharopolyspora strain according to claim 33, wherein steps d)-e) are repeated until the phenotypic performance of a Saccharopolyspora strain of a subsequent library exhibits at least a one-fold increase in a measured phenotypic variable compared to the phenotypic performance of the production Saccharopolyspora strain.

45. The method for rehabilitating and improving the phenotypic performance of a production Saccharopolyspora strain according to claim 33, wherein the improved phenotypic performance of step f) is selected from the group consisting of: volumetric productivity of a product of interest, specific productivity of a product of interest, yield of a product of interest, titer of a product of interest, and combinations thereof.

46. The method for rehabilitating and improving the phenotypic performance of a production Saccharopolyspora strain according to claim 33, wherein the improved phenotypic performance of step f) is: increased or more efficient production of a product of interest, said product of interest selected from the group consisting of: a small molecule, enzyme, peptide, amino acid, organic acid, synthetic compound, fuel, alcohol, primary extracellular metabolite, secondary extracellular metabolite, intracellular component molecule, and combinations thereof.

47. The method for rehabilitating and improving the phenotypic performance of a production Saccharopolyspora strain according to claim 46, wherein the product of interest is selected from the group consisting of a spinosyn, spinosad, spinetoram, genistein, choline oxidase, a coumamidine compound, erythromycin, ivermectin aglycone, a HMG-CoA reductase inhibitor, a carboxylic acid isomer, alpha-methyl methionine, thialysine, alpha-ketobytarate, aspartate hydoxymate, azaserine, 5-fuoroindole, beta-hydroxynorvaline, cerulenin, purine, pyrimidine, and analogs thereof.

48. The method for rehabilitating and improving the phenotypic performance of a production Saccharopolyspora strain according to claim 46, wherein the spinosyn is spinosyn A, spinosyn D, spinosyn J, spinosyn L, or combinations thereof.

49. The method for rehabilitating and improving the phenotypic performance of a production Saccharopolyspora strain according to claim 33, wherein the identified genetic variations further comprise artificial promoter swap genetic variations from a promoter swap library.

50. The method for rehabilitating and improving the phenotypic performance of a production Saccharopolyspora strain according to claim 33, further comprising engineering the genome of at least one microbial strain of either the initial library of Saccharopolyspora strains, or a subsequent library of Saccharopolyspora strains, to comprise one or more promoters from a promoter ladder operably linked to an endogenous Saccharopolyspora target gene.

51. The method for rehabilitating and improving the phenotypic performance of a production Saccharopolyspora strain according to claim 33, wherein the strain library comprises at least one library selected from the group consisting of a promoter swap microbial strain library, SNP swap microbial strain library, start/stop codon microbial strain library, optimized sequence microbial strain library, a terminator swap microbial strain library, a transposon mutagenesis microbial strain diversity library, a ribosomal binding site microbial strain library, an anti-metabolite/fermentation product resistance library, a termination insertion microbial strain library, and any combination thereof.

52. The method for rehabilitating and improving the phenotypic performance of a production Saccharopolyspora strain according to claim 51, wherein the strain library comprises at least one library selected from the group consisting of:
1) a promoter swap microbial strain library comprising at least one promoter having a sequence selected from SEQ ID No. 1-69;
2) a terminator swap microbial strain library comprising at least one terminator having a sequence selected from SEQ ID Nos. 70 to 80; and
3) a ribosomal binding site (R BS) library comprising at least one RBS having a sequence selected from SEQ ID Nos. 97 to 127.

53. A method for generating a promoter swap Saccharopolyspora strain library, said method comprising the steps of:
a. providing a plurality of target genes endogenous to a base Saccharopolyspora strain, and a promoter ladder, wherein said promoter ladder comprises a plurality of promoters exhibiting different expression profiles in the base Saccharopolyspora strain; and
b. engineering the genome of the base Saccharopolyspora strain, to thereby create an initial promoter swap Saccharopolyspora strain library comprising a plurality of individual Saccharopolyspora strains with unique genetic variations found within each strain of said plurality of individual Saccharopolyspora strains, wherein each of said unique genetic variations comprises one or more of the promoters from the promoter ladder operably linked to one of the target genes endogenous to the base Saccharopolyspora strain.

54. The method for generating a promoter swap Saccharopolyspora strain library according to claim 53, wherein at least one of the plurality of promoters comprises a promoter having a sequence selected from SEQ ID No. 1-69.

55. A promoter swap method for improving the phenotypic performance of a production Saccharopolyspora strain, comprising the steps of:
a. providing a plurality of target genes endogenous to a base Saccharopolyspora strain, and a promoter ladder, wherein said promoter ladder comprises a plurality of promoters exhibiting different expression profiles in the base Saccharopolyspora strain;
b. engineering the genome of the base Saccharopolyspora strain, to thereby create an initial promoter swap Saccharopolyspora strain library comprising a plurality of individual Saccharopolyspora strains with unique genetic variations found within each strain of said plurality of individual Saccharopolyspora strains, wherein each of said unique genetic variations comprises one or more of the promoters from the promoter ladder operably linked to one of the target genes endogenous to the base Saccharopolyspora strain;
c. screening and selecting individual Saccharopolyspora strains of the initial promoter swap Saccharopolyspora strain library for phenotypic performance improvements over a reference Saccharopolyspora strain, thereby identifying unique genetic variations that confer the phenotypic performance improvements;
d. providing a subsequent plurality of Saccharopolyspora microbes that each comprise a combination of unique genetic variations from the genetic variations present in at least two individual Saccharopolyspora strains screened in the preceding step, to thereby create a subsequent promoter swap Saccharopolyspora strain library;
e. screening and selecting individual Saccharopolyspora strains of the subsequent promoter swap Saccharopolyspora strain library for the desired phenotypic performance improvements over the reference E. coli strain, thereby identifying unique combinations of genetic variation that confer additional phenotypic performance improvements; and
f. repeating steps d)-e) one or more times, in a linear or non-linear fashion, until a Saccharopolyspora strain exhibits a desired level of improved phenotypic performance compared to the phenotypic performance of the production Saccharopolyspora strain, wherein each subsequent iteration creates a new promoter swap Saccharopolyspora strain library of Saccharopolyspora strains, wherein each strain in the new library comprises genetic variations that are a combination of genetic variations selected from amongst at least two individual Saccharopolyspora strains of a preceding promoter swap Saccharopolyspora strain library.

56. The promoter swap method for improving the phenotypic performance of a production Saccharopolyspora strain according to claim 55, wherein the subsequent promoter swap Saccharopolyspora strain library is a full combinatorial library of the initial promoter swap Saccharopolyspora strain library.

57. The promoter swap method for improving the phenotypic performance of a production Saccharopolyspora strain according to claim 55, wherein the subsequent promoter swap Saccharopolyspora strain library is a full combinatorial library of the initial promoter swap Saccharopolyspora strain library.

58. The promoter swap method for improving the phenotypic performance of a production Saccharopolyspora strain according to claim 55, wherein the subsequent promoter swap Saccharopolyspora strain library is a subset of a full combinatorial library of the initial promoter swap Saccharopolyspora strain library.

59. The promoter swap method for improving the phenotypic performance of a production Saccharopolyspora strain according to claim 55, wherein the subsequent promoter swap Saccharopolyspora strain library is a full combinatorial library of a preceding promoter swap Saccharopolyspora strain library.

60. The promoter swap method for improving the phenotypic performance of a production Saccharopolyspora strain according to claim 55, wherein the subsequent promoter swap Saccharopolyspora strain library is a subset of a full combinatorial library of a preceding promoter swap Saccharopolyspora strain library.

61. The promoter swap method for improving the phenotypic performance of a production Saccharopolyspora strain according to claim 55, wherein steps d)-e) are repeated until the phenotypic performance a Saccharopolyspora strain of a subsequent promoter swap Saccharopolyspora strain library exhibits at least a 10% increase in a measured phenotypic variable compared to the phenotypic performance of the production Saccharopolyspora strain.

62. The promoter swap method for improving the phenotypic performance of a production Saccharopolyspora strain according to claim 55, wherein steps d)-e) are repeated until the phenotypic performance of a Saccharopolyspora strain of a subsequent promoter swap Saccharopolyspora strain library exhibits at least a one-fold increase in a measured phenotypic variable compared to the phenotypic performance of the production Saccharopolyspora strain.

63. The promoter swap method for improving the phenotypic performance of a production Saccharopolyspora strain according to claim 55, wherein the improved phenotypic performance of step f) is selected from the group consisting of: volumetric productivity of a product of interest, specific productivity of a product of interest, yield of a product of interest, titer of a product of interest, and combinations thereof.

64. The promoter swap method for improving the phenotypic performance of a production Saccharopolyspora strain according to claim 55, wherein the improved phenotypic performance of step f) is: increased or more efficient production of a product of interest, said product of interest selected from the group consisting of: a small molecule, enzyme, peptide, amino acid, organic acid, synthetic compound, fuel, alcohol, primary extracellular metabolite, secondary extracellular metabolite, intracellular component molecule, and combinations thereof.

65. The promoter swap method for improving the phenotypic performance of a production Saccharopolyspora strain according to claim 64, wherein the product of interest is selected from the group consisting of a spinosyn, spinosad, spinetoram, genistein, choline oxidase, a coumamidine compound, erythromycin, ivermectin aglycone, a HMG-CoA reductase inhibitor, a carboxylic acid isomer, alpha-methyl methionine, thialysine, alpha-ketobytarate, aspartate hydoxymate, azaserine, 5-fuoroindole, beta-hydroxynorvaline, cerulenin, purine, pyrimidine, and analogs thereof.

66. The promoter swap method for improving the phenotypic performance of a production Saccharopolyspora strain according to claim 65, wherein the spinosyn is spinosyn A, spinosyn D, spinosyn J, spinosyn L, or combinations thereof.

67. The promoter swap method for improving the phenotypic performance of a production Saccharopolyspora strain according to claim 55, wherein the promoter ladder comprises at least one promoter with a nucleotide sequence selected from SEQ ID No. 1-69.

68. A method for generating a terminator swap Saccharopolyspora strain library, comprising the steps of:
a. providing a plurality of target genes endogenous to a base Saccharopolyspora strain, and a terminator ladder, wherein said terminator ladder comprises a plurality of terminators exhibiting different expression profiles in the base Saccharopolyspora strain; and
b. engineering the genome of the base Saccharopolyspora strain, to thereby create an initial terminator swap Saccharopolyspora strain library comprising a plurality of individual Saccharopolyspora strains with unique genetic variations found within each strain of said plurality of individual Saccharopolyspora strains, wherein each of said unique genetic variations comprises one or more of the terminators from the terminator ladder operably linked to one of the target genes endogenous to the base Saccharopolyspora strain.

69. A terminator swap method for improving the phenotypic performance of a production Saccharopolyspora strain, comprising the steps of:
a. providing a plurality of target genes endogenous to a base Saccharopolyspora strain, and a terminator ladder, wherein said terminator ladder comprises a plurality of terminators exhibiting different expression profiles in the base Saccharopolyspora strain;
b. engineering the genome of the base Saccharopolyspora strain, to thereby create an initial terminator swap Saccharopolyspora strain library comprising a plurality of individual Saccharopolyspora strains with unique genetic variations found within each strain of said plurality of individual Saccharopolyspora strains, wherein each of said unique genetic variations comprises one or more of the terminators from the terminator ladder operably linked to one of the target genes endogenous to the base Saccharopolyspora strain;
c. screening and selecting individual Saccharopolyspora strains of the initial terminator swap Saccharopolyspora strain library for phenotypic performance improvements over a reference Saccharopolyspora strain, thereby identifying unique genetic variations that confer phenotypic performance improvements;
d. providing a subsequent plurality of Saccharopolyspora microbes that each comprise a combination of unique genetic variations from the genetic variations present in at least two individual Saccharopolyspora strains screened in the preceding step, to thereby create a subsequent terminator swap Saccharopolyspora strain library;
e. screening and selecting individual Saccharopolyspora strains of the subsequent terminator swap Saccharopolyspora strain library for phenotypic performance improvements over the reference Saccharopolyspora strain, thereby identifying unique combinations of genetic variation that confer additional phenotypic performance improvements; and
f. repeating steps d)-e) one or more times, in a linear or non-linear fashion, until a Saccharopolyspora strain exhibits a desired level of improved phenotypic performance compared to the phenotypic performance of the production Saccharopolyspora strain, wherein each subsequent iteration creates a new terminator swap Saccharopolyspora strain library of microbial strains, where each strain in the new library comprises genetic variations that are a combination of genetic variations selected from amongst at least two individual Saccharopolyspora strains of a preceding library.

70. The terminator swap method for improving the phenotypic performance of a production Saccharopolyspora strain according to claim 69, wherein the subsequent terminator swap Saccharopolyspora strain library is a full combinatorial library of the initial terminator swap Saccharopolyspora strain library.

71. The terminator swap method for improving the phenotypic performance of a production Saccharopolyspora strain according to claim 69, wherein the subsequent terminator swap Saccharopolyspora strain library is a subset of a full combinatorial library of the initial terminator swap Saccharopolyspora strain library.

72. The terminator swap method for improving the phenotypic performance of a production Saccharopolyspora strain according to claim 69, wherein the subsequent terminator swap Saccharopolyspora strain library is a full combinatorial library of a preceding terminator swap Saccharopolyspora strain library.

73. The terminator swap method for improving the phenotypic performance of a production Saccharopolyspora strain according to claim 69, wherein the subsequent terminator swap Saccharopolyspora strain library is a subset of a full combinatorial library of a preceding terminator swap Saccharopolyspora strain library.

74. The terminator swap method for improving the phenotypic performance of a production Saccharopolyspora strain according to claim 69, wherein steps d)-e) are repeated until the phenotypic performance of a Saccharopolyspora strain of a subsequent terminator swap Saccharopolyspora strain library exhibits at least a 10% increase in a measured phenotypic variable compared to the phenotypic performance of the production Saccharopolyspora strain.

75. The terminator swap method for improving the phenotypic performance of a production Saccharopolyspora strain according to claim 69, wherein steps d)-e) are repeated until the phenotypic performance of a Saccharopolyspora strain of a subsequent terminator swap Saccharopolyspora strain library exhibits at least a one-fold increase in a measured phenotypic variable compared to the phenotypic performance of the production Saccharopolyspora strain.

76. The terminator swap method for improving the phenotypic performance of a production Saccharopolyspora strain according to claim 69, wherein the improved phenotypic performance of step f) is selected from the group consisting of: volumetric productivity of a product of interest, specific productivity of a product of interest, yield of a product of interest, titer of a product of interest, and combinations thereof.

77. The terminator swap method for improving the phenotypic performance of a production Saccharopolyspora strain according to claim 69, wherein the improved phenotypic performance of step f) is: increased or more efficient production of a product of interest, said product of interest selected from the group consisting of: a small molecule, enzyme, peptide, amino acid, organic acid, synthetic compound, fuel, alcohol, primary extracellular metabolite, secondary extracellular metabolite, intracellular component molecule, and combinations thereof.

78. The terminator swap method for improving the phenotypic performance of a production Saccharopolyspora strain according to claim 77, wherein the product of interest is selected from the group consisting of a spinosyn, spinosad, spinetoram, genistein, choline oxidase, a coumamidine compound, erythromycin, ivermectin aglycone, a HMG-CoA reductase inhibitor, a carboxylic acid isomer, alpha-methyl methionine, thialysine, alpha-ketobytarate, aspartate hydoxymate, azaserine, 5-fuoroindole, beta-hydroxynorvaline, cerulenin, purine, pyrimidine, and analogs thereof.

79. The terminator swap method for improving the phenotypic performance of a production Saccharopolyspora strain according to claim 78, wherein the spinosyn is spinosyn A, spinosyn D, spinosyn J, spinosyn L, or combinations thereof.

80. The terminator swap method for improving the phenotypic performance of a production Saccharopolyspora strain according to claim 69, wherein the terminator ladder comprises at least one terminator with a nucleotide sequence selected from SEQ ID No. 70-80.

81. A method for generating a ribosomal binding site (RBS) Saccharopolyspora strain library, comprising the steps of:
a. providing a plurality of target genes endogenous to a base Saccharopolyspora strain, and a RBS ladder, wherein said RBS ladder comprises a plurality of RBSs exhibiting different expression profiles in the base Saccharopolyspora strain; and
b. engineering the genome of the base Saccharopolyspora strain, to thereby create an initial RBS Saccharopolyspora strain library comprising a plurality of individual Saccharopolyspora strains with unique genetic variations found within each strain of said plurality of individual Saccharopolyspora strains, wherein each of said unique genetic variations comprises one or more of the RBSs from the RBS ladder operably linked to one of the target genes endogenous to the base Saccharopolyspora strain.

82. A method for improving the phenotypic performance of a production Saccharopolyspora strain, comprising the steps of:
a. providing a plurality of target genes endogenous to a base Saccharopolyspora strain, and a RBS ladder, wherein said RBS ladder comprises a plurality of RBSs exhibiting different expression profiles in the base Saccharopolyspora strain;
b. engineering the genome of the base Saccharopolyspora strain, to thereby create an initial RBS Saccharopolyspora strain library comprising a plurality of individual Saccharopolyspora strains with unique genetic variations found within each strain of said plurality of individual Saccharopolyspora strains, wherein each of said unique genetic variations comprises one or more of the RBSs from the RBS ladder operably linked to one of the target genes endogenous to the base Saccharopolyspora strain;
c. screening and selecting individual Saccharopolyspora strains of the initial RBS Saccharopolyspora strain library for phenotypic performance improvements over a reference Saccharopolyspora strain, thereby identifying unique genetic variations that confer phenotypic performance improvements;
d. providing a subsequent plurality of Saccharopolyspora strains that each comprise a combination of unique genetic variations from the genetic variations present in at least two individual Saccharopolyspora strains screened in the preceding step, to thereby create a subsequent RBS Saccharopolyspora strain library;
e. screening and selecting individual Saccharopolyspora strains of the subsequent RBS Saccharopolyspora strain library for phenotypic performance improvements over the reference Saccharopolyspora strain, thereby identifying unique combinations of genetic variation that confer additional phenotypic performance improvements; and
f. repeating steps d)-e) one or more times, in a linear or non-linear fashion, until a Saccharopolyspora strain exhibits a desired level of improved phenotypic performance compared to the phenotypic performance of the production Saccharopolyspora strain, wherein each subsequent iteration creates a new RBS Saccharopolyspora strain library of microbial strains, where each strain in the new library comprises genetic variations that are a combination of genetic variations selected from amongst at least two individual Saccharopolyspora strains of a preceding library.

83. The method for improving the phenotypic performance of a production Saccharopolyspora strain according to claim 82, wherein the subsequent RBS Saccharopolyspora strain library is a full combinatorial library of the initial RBS Saccharopolyspora strain library.

84. The method for improving the phenotypic performance of a production Saccharopolyspora strain according to claim 82, wherein the subsequent RBS Saccharopolyspora strain library is a subset of a full combinatorial library of the initial RBS Saccharopolyspora strain library.

85. The method for improving the phenotypic performance of a production Saccharopolyspora strain according to claim 82, wherein the subsequent RBS Saccharopolyspora strain library is a full combinatorial library of a preceding RBS Saccharopolyspora strain library.

86. The method for improving the phenotypic performance of a production Saccharopolyspora strain according to claim 82, wherein the subsequent RBS Saccharopolyspora strain library is a subset of a full combinatorial library of a preceding RBS Saccharopolyspora strain library.

87. The method for improving the phenotypic performance of a production Saccharopolyspora strain according to claim 82, wherein steps d)-e) are repeated until the phenotypic performance of a Saccharopolyspora strain of a subsequent RBS Saccharopolyspora strain library exhibits at least a 10% increase in a measured phenotypic variable compared to the phenotypic performance of the production Saccharopolyspora strain.

88. The method for improving the phenotypic performance of a production Saccharopolyspora strain according to claim 82, wherein steps d)-e) are repeated until the phenotypic performance of a Saccharopolyspora strain of a subsequent RBS Saccharopolyspora strain library exhibits at least a one-fold increase in a measured phenotypic variable compared to the phenotypic performance of the production Saccharopolyspora strain.

89. The method for improving the phenotypic performance of a production Saccharopolyspora strain according to claim 82, wherein the improved phenotypic performance of step f) is selected from the group consisting of: volumetric productivity of a product of interest, specific productivity of a product of interest, yield of a product of interest, titer of a product of interest, and combinations thereof.

90. The method for improving the phenotypic performance of a production Saccharopolyspora strain according to claim 82, wherein the improved phenotypic performance of step f) is: increased or more efficient production of a product of interest, said product of interest selected from the group consisting of: a small molecule, enzyme, peptide, amino acid, organic acid, synthetic compound, fuel, alcohol, primary extracellular metabolite, secondary extracellular metabolite, intracellular component molecule, and combinations thereof.

91. The method for improving the phenotypic performance of a production Saccharopolyspora strain according to claim 90, wherein the product of interest is selected from the group consisting of a spinosyn, spinosad, spinetoram, genistein, choline oxidase, a coumamidine compound, erythromycin, ivermectin aglycone, a HMG-CoA reductase inhibitor, a carboxylic acid isomer, alpha-methyl methionine, thialysine, alpha-ketobytarate, aspartate hydoxymate, azaserine, 5-fuoroindole, beta-hydroxynorvaline, cerulenin, purine, pyrimidine, and analogs thereof.

92. The method for improving the phenotypic performance of a production Saccharopolyspora strain according to claim 91, wherein the spinosyn is spinosyn A, spinosyn D, spinosyn J, spinosyn L, or combinations thereof.

93. The method for improving the phenotypic performance of a production Saccharopolyspora strain according to claim 82, wherein the RBS ladder comprises at least one RBS with a nucleotide sequence selected from SEQ ID No. 97-127.

94. A method for generating a transposon mutagenesis Saccharopolyspora strain diversity library, comprising
a) introducing a transposon into a population of cells of one or more base Saccharopolyspora strains; and
b) selecting for Saccharopolyspora strain comprising randomly integrated transposon, thereby creating an initial Saccharopolyspora strain library comprising a plurality of individual Saccharopolyspora strains with unique genetic variations found within each strain of said plurality of individual Saccharopolyspora strains, wherein each of said unique genetic variations comprises one or more randomly integrated transposon.

95. The method of claim 94, further comprising:
c). selecting for a subsequence Saccharopolyspora strain library exhibits at least one increase in a measured phenotypic variable compared to the phenotypic performance of the base Saccharopolyspora strain.

96. The method of claim 94, wherein the transposon is introduced into the base Saccharopolyspora strain using a complex of transposon and transposase protein which allows for in vivo transposition of the transposon into the genome of the Saccharopolyspora strain.

97. The method of claim 94, wherein the transposase protein is derived from EZ-Tn5 transposome system.

98. The method of claim 94, wherein the transposon is a Loss-of-Function (LoF) transposon, or a Gain-of-Function (GoF) transposon.

99. The method of claim 94, wherein the GoF transposon comprises a solubility tag, a promoter, and/or a counter-selection marker.

100. A method for improving the phenotypic performance of a production Saccharopolyspora strain, comprising the steps of:
a. engineering the genome of a base Saccharopolyspora strain by transposon mutagenesis, to thereby create an initial transposon mutagenesis Saccharopolyspora strain library comprising a plurality of individual Saccharopolyspora strains with unique genetic variations found within each strain of said plurality of individual Saccharopolyspora strains, wherein each of said unique genetic variations comprises one or more transposon;
b. screening and selecting individual Saccharopolyspora strains of the initial transposon mutagenesis Saccharopolyspora strain library for phenotypic performance improvements over a reference Saccharopolyspora strain, thereby identifying unique genetic variations that confer phenotypic performance improvements;
c. providing a subsequent plurality of Saccharopolyspora strains that each comprise a combination of unique genetic variations from the genetic variations present in at least two individual Saccharopolyspora strains screened in the preceding step, to thereby create a subsequent transposon mutagenesis Saccharopolyspora strain library;
d. screening and selecting individual Saccharopolyspora strains of the subsequent transposon mutagenesis Saccharopolyspora strain library for phenotypic performance improvements over the reference Saccharopolyspora strain, thereby identifying unique combinations of genetic variation that confer additional phenotypic performance improvements; and
e. repeating steps c)-d) one or more times, in a linear or non-linear fashion, until a Saccharopolyspora strain exhibits a desired level of improved phenotypic performance compared to the phenotypic performance of the production Saccharopolyspora strain, wherein each subsequent iteration creates a new transposon mutagenesis Saccharopolyspora strain library of microbial strains, where each strain in the new library comprises genetic variations that are a combination of genetic variations selected from amongst at least two individual Saccharopolyspora strains of a preceding library.

101. The method for improving the phenotypic performance of a production Saccharopolyspora strain according to claim 100, wherein the subsequent transposon mutagenesis Saccharopolyspora strain library is a full combinatorial library of the initial transposon mutagenesis Saccharopolyspora strain library.

102. The method for improving the phenotypic performance of a production Saccharopolyspora strain according to claim 100, wherein the subsequent transposon mutagenesis Saccharopolyspora strain library is a subset of a full combinatorial library of the initial transposon mutagenesis Saccharopolyspora strain library.

103. The method for improving the phenotypic performance of a production Saccharopolyspora strain according to claim 100, wherein the subsequent transposon mutagenesis Saccharopolyspora strain library is a full combinatorial library of a preceding transposon mutagenesis Saccharopolyspora strain library.

104. The method for improving the phenotypic performance of a production Saccharopolyspora strain according to claim 100, wherein the subsequent transposon mutagenesis Saccharopolyspora strain library is a subset of a full combinatorial library of a preceding transposon mutagenesis Saccharopolyspora strain library.

105. The method for improving the phenotypic performance of a production Saccharopolyspora strain according to claim 100, wherein steps c)-d) are repeated until the phenotypic performance of a Saccharopolyspora strain of a subsequent transposon mutagenesis Saccharopolyspora strain library exhibits at least a 10% increase in a measured phenotypic variable compared to the phenotypic performance of the production Saccharopolyspora strain.

106. The method for improving the phenotypic performance of a production Saccharopolyspora strain according to claim 100, wherein steps c)-d) are repeated until the phenotypic performance of a Saccharopolyspora strain of a subsequent transposon mutagenesis Saccharopolyspora strain library exhibits at least a one-fold increase in a measured phenotypic variable compared to the phenotypic performance of the production Saccharopolyspora strain.

107. The method for improving the phenotypic performance of a production Saccharopolyspora strain according to claim 100, wherein the improved phenotypic performance of step e) is selected from the group consisting of: volumetric productivity of a product of interest, specific productivity of a product of interest, yield of a product of interest, titer of a product of interest, and combinations thereof.

108. The method for improving the phenotypic performance of a production Saccharopolyspora strain according to claim 100, wherein the improved phenotypic performance of step e) is: increased or more efficient production of a product of interest, said product of interest selected from the group consisting of: a small molecule, enzyme, peptide, amino acid, organic acid, synthetic compound, fuel, alcohol, primary extracellular metabolite, secondary extracellular metabolite, intracellular component molecule, and combinations thereof.

109. The method for improving the phenotypic performance of a production Saccharopolyspora strain according to claim 108, wherein the product of interest is selected from the group consisting of a spinosyn, spinosad, spinetoram, genistein, choline oxidase, a coumamidine compound, erythromycin, ivermectin aglycone, a HMG-CoA reductase inhibitor, a carboxylic acid isomer, alpha-methyl methionine, thialysine, alpha-ketobytarate, aspartate hydoxymate, azaserine, 5-fuoroindole, beta-hydroxynorvaline, cerulenin, purine, pyrimidine, and analogs thereof.

110. The method for improving the phenotypic performance of a production Saccharopolyspora strain according to claim 109, wherein the spinosyn is spinosyn A, spinosyn D, spinosyn J, spinosyn L, or combinations thereof.

111. The method for improving the phenotypic performance of a production Saccharopolyspora strain according to claim 100, wherein the transposon comprises is a Loss-of-Function (LoF) transposon, or a Gain-of-Function (GoF) transposon.

112. The method of claim 111, wherein the GoF transposon comprises a solubility tag, a promoter, and/or a counter-selection marker.

113. A method for generating an anti-metabolite/fermentation product resistant Saccharopolyspora strain library, comprising the step of:
a) selecting for Saccharopolyspora strains resistant to a predetermined metabolite and/or a fermentation product, thereby creating an initial Saccharopolyspora strain library comprising a plurality of individual Saccharopolyspora strains with unique genetic variations found within each strain of said plurality of individual Saccharopolyspora strains, wherein at least one of said unique genetic variations results in resistance to the predetermined metabolite and/or a fermentation product; and
b) collecting Saccharopolyspora strains resistant to the predetermined metabolite and/or the fermentation product to generate the anti-metabolite/fermentation product resistant Saccharopolyspora strain library.

114. The method for generating an anti-metabolite/fermentation product resistant Saccharopolyspora strain library of claim 113, wherein the predetermined metabolite and/or fermentation product is selected from the group consisting of molecules involved in the spinosyn synthesis pathway, molecules involved in the SAM/methionine pathway, molecules involved in the lysine production pathway, molecules involved in the tryptophan pathway, molecules involved in the threonine pathway, molecules involved in the acetyl-CoA production pathway, and molecules involved in the de-novo or salvage purine and pyrimidine pathways.

115. The method for generating an anti-metabolite/fermentation product resistant Saccharopolyspora strain library of claim 114, wherein:
1) the molecule involved in the spinosyn synthesis pathway is a spinosyn, and optionally each strain is resistant to about 50 ug/ml to about 2 mg/ml spinosyn J/L;
2) the molecule involved in the SAM/methionine pathway is alpha-methyl methionine (aMM) or norleucine, and optionally each strain is resistant to about 1 mM to about 5 mM alpha-methyl methionine (aMM);
3) the molecule involved in the lysine production pathway is thialysine or a mixture of alpha-ketobytarate and aspartate hydoxymate;
4) the molecule involved in the tryptophan pathway is azaserine or 5-fuoroindole;
5) the molecule involved in the threonine pathway is beta-hydroxynorvaline;
6) the molecule involved in the acetyl-CoA production pathway is cerulenin, and
7) the molecule involved in the de-novo or salvage purine and pyrimidine pathways is a purine or a pyrimidine analog.

116. The method for generating an anti-metabolite/fermentation product resistant Saccharopolyspora strain library of claim 113, further comprising the step of:
b). selecting for a subsequence Saccharopolyspora strain library exhibits at least one increase in a measured phenotypic variable compared to the phenotypic performance of the base Saccharopolyspora strain.

117. The method for generating an anti-metabolite/fermentation product resistant Saccharopolyspora strain library of claim 116, wherein each strain in the subsequence Saccharopolyspora strain library exhibits an increased synthesis of a spinosyn.

118. A method for improving the phenotypic performance of a production Saccharopolyspora strain, comprising the steps of:
a) providing an initial anti-metabolite/fermentation product resistant Saccharopolyspora strain library comprising a plurality of individual Saccharopolyspora strains with unique genetic variations found within each strain of said plurality of individual Saccharopolyspora strains, wherein each of said unique genetic variations comprises one or more of genetic variations, wherein the genetic variations confer resistance to a predetermined metabolite or a fermentation product;
b) screening and selecting individual Saccharopolyspora strains of the initial anti-metabolite/fermentation product resistant Saccharopolyspora strain library for phenotypic performance improvements over a reference Saccharopolyspora strain, thereby identifying unique genetic variations that confer phenotypic performance improvements;
c) providing a subsequent plurality of Saccharopolyspora strains that each comprise a combination of unique genetic variations from the genetic variations present in at least two individual Saccharopolyspora strains screened in the preceding step, to thereby create a subsequent anti-metabolite/fermentation product resistant Saccharopolyspora strain library;
d) screening and selecting individual Saccharopolyspora strains of the subsequent anti-metabolite/fermentation product resistant Saccharopolyspora strain library for phenotypic performance improvements over the reference Saccharopolyspora strain, thereby identifying unique combinations of genetic variation that confer additional phenotypic performance improvements; and
e) repeating steps c)-d) one or more times, in a linear or non-linear fashion, until a Saccharopolyspora strain exhibits a desired level of improved phenotypic performance compared to the phenotypic performance of the production Saccharopolyspora strain, wherein each subsequent iteration creates a new anti-metabolite/fermentation product resistant Saccharopolyspora strain library of microbial strains, where each strain in the new library comprises genetic variations that are a combination of genetic variations selected from amongst at least two individual Saccharopolyspora strains of a preceding library.

119. The method for improving the phenotypic performance of a production Saccharopolyspora strain according to claim 118, wherein the subsequent anti-metabolite/fermentation product resistant Saccharopolyspora strain library is a full combinatorial library of the initial anti-metabolite/fermentation product resistant Saccharopolyspora strain library.

120. The method for improving the phenotypic performance of a production Saccharopolyspora strain according to claim 118, wherein the subsequent anti-metabolite/fermentation product resistant Saccharopolyspora strain library is a subset of a full combinatorial library of the initial anti-metabolite/fermentation product resistant Saccharopolyspora strain library.

121. The method for improving the phenotypic performance of a production Saccharopolyspora strain according to claim 118, wherein the subsequent anti-metabolite/fermentation product resistant Saccharopolyspora strain library is a full combinatorial library of a preceding anti-metabolite/fermentation product resistant Saccharopolyspora strain library.

122. The method for improving the phenotypic performance of a production Saccharopolyspora strain according to claim 118, wherein the subsequent anti-metabolite/fermentation product resistant Saccharopolyspora strain library is a subset of a full combinatorial library of a preceding anti-metabolite/fermentation product resistant Saccharopolyspora strain library.

123. The method for improving the phenotypic performance of a production Saccharopolyspora strain according to claim 118, wherein steps c)-d) are repeated until the phenotypic performance of a Saccharopolyspora strain of a subsequent anti-metabolite/fermentation product resistant Saccharopolyspora strain library exhibits at least a 10% increase in a measured phenotypic variable compared to the phenotypic performance of the production Saccharopolyspora strain.

124. The method for improving the phenotypic performance of a production Saccharopolyspora strain according to claim 118, wherein steps c)-d) are repeated until the phenotypic performance of a Saccharopolyspora strain of a subsequent anti-metabolite/fermentation product resistant Saccharopolyspora strain library exhibits at least a one-fold increase in a measured phenotypic variable compared to the phenotypic performance of the production Saccharopolyspora strain.

125. The method for improving the phenotypic performance of a production Saccharopolyspora strain according to claim 118, wherein the improved phenotypic performance of step e) is selected from the group consisting of: volumetric productivity of a product of interest, specific productivity of a product of interest, yield of a product of interest, titer of a product of interest, and combinations thereof.

126. The method for improving the phenotypic performance of a production Saccharopolyspora strain according to claim 125, wherein the improved phenotypic performance of step e) is: increased or more efficient production of a product of interest, said product of interest selected from the group consisting of: a small molecule, enzyme, peptide, amino acid, organic acid, synthetic compound, fuel, alcohol, primary extracellular metabolite, secondary extracellular metabolite, intracellular component molecule, and combinations thereof.

127. The method for improving the phenotypic performance of a production Saccharopolyspora strain according to claim 126, wherein the product of interest is selected from the group consisting of a spinosyn, spinosad, spinetoram, genistein, choline oxidase, a coumamidine compound, erythromycin, ivermectin aglycone, a HMG-CoA reductase inhibitor, a carboxylic acid isomer, alpha-methyl methionine, thialysine, alpha-ketobytarate, aspartate hydoxymate, azaserine, 5-fuoroindole, beta-hydroxynorvaline, cerulenin, purine, pyrimidine, and analogs thereof.

128. The method for improving the phenotypic performance of a production Saccharopolyspora strain according to claim 127, wherein the spinosyn is spinosyn A, spinosyn D, spinosyn J, spinosyn L, or combinations thereof.

129. A Saccharopolyspora host cell comprising a promoter operably linked to an endogenous gene of the host cell, wherein the promoter is heterologous to the endogenous gene, wherein the promoter has a sequence selected from the group consisting of SEQ ID Nos. 1-69.

130. The Saccharopolyspora host cell of claim 129, wherein the endogenous gene is involved in synthesis of a spinosyn in the Saccharopolyspora host cell.

131. The Saccharopolyspora host cell of claim 129, wherein Saccharopolyspora host cell has a desired level of improved phenotypic performance compared to the phenotypic performance of a reference Saccharopolyspora strain without the promoter operably linked to the endogenous gene.

132. A Saccharopolyspora strain library, wherein each Saccharopolyspora strain in the library comprises a promoter operably linked to an endogenous gene of the host cell, wherein the promoter is heterologous to the endogenous gene, wherein the promoter has a sequence selected from the group consisting of SEQ ID Nos. 1-69.

133. A Saccharopolyspora host cell comprising a terminator linked to an endogenous gene of the host cell, wherein the terminator is heterologous to the endogenous gene, wherein the promoter has a sequence selected from the group consisting of SEQ ID Nos. 70-80.

134. The Saccharopolyspora host cell of claim 133, wherein the endogenous gene is involved in synthesis of a spinosyn in the Saccharopolyspora host cell.

135. The Saccharopolyspora host cell of claim 133, wherein Saccharopolyspora host cell has a desired level of improved phenotypic performance compared to the phenotypic performance of a reference Saccharopolyspora strain without the promoter operably linked to the endogenous gene.

136. A Saccharopolyspora strain library, wherein each Saccharopolyspora strain in the library comprises a terminator linked to an endogenous gene of the host cell, wherein the terminator is heterologous to the endogenous gene, wherein the terminator has a sequence selected from the group consisting of SEQ ID Nos. 70-80.

137. A Saccharopolyspora host cell comprising a ribosomal binding site operably linked to an endogenous gene of the host cell, wherein the ribosomal binding site is heterologous to the endogenous gene, wherein the ribosomal binding site has a sequence selected from the group consisting of SEQ ID Nos. 97-127.

138. The Saccharopolyspora host cell of claim 137, wherein the endogenous gene is involved in synthesis of a spinosyn in the Saccharopolyspora host cell.

139. The Saccharopolyspora host cell of claim 137, wherein Saccharopolyspora host cell has a desired level of improved phenotypic performance compared to the phenotypic performance of a reference Saccharopolyspora strain without the RBS operably linked to the endogenous gene.

140. A Saccharopolyspora strain library, wherein each Saccharopolyspora strain in the library comprises a ribosomal binding site operably linked to an endogenous gene of the host cell, wherein the ribosomal binding site is heterologous to the endogenous gene, wherein the ribosomal binding site has a sequence selected from the group consisting of SEQ ID Nos. 97-127.

141. A Saccharopolyspora host cell comprising a transposon, wherein Saccharopolyspora host cell has a desired level of improved phenotypic performance compared to the phenotypic performance of a reference Saccharopolyspora strain without the transposon.

142. The Saccharopolyspora host cell of claim 141, wherein the transposon is a Loss-of-Function (LoF) transposon, or a Gain-of-Function (GoF) transposon.

143. The Saccharopolyspora host cell of claim 142, wherein the Gain-of-Function (GoF) transposon comprises a promoter, a counterselection marker, and/or a solubility tag.

144. The Saccharopolyspora host cell of claim 141, wherein the transposon comprises a sequence selected from the group consisting of SEQ ID No. 128-131.

145. A Saccharopolyspora strain library, wherein each Saccharopolyspora strain in the library comprises a transposon having a sequence selected from the group consisting of SEQ ID No. 128-131, wherein the transposon in each strain is at a different genomic locus.

146. A Saccharopolyspora strain library, wherein each Saccharopolyspora strain in the library comprises a genetic variation that results in resistance of the strain to
1) a molecule involved in the spinosyn synthesis pathway,
2) a molecule involved in the SAM/methionine pathway,
3) a molecule involved in the lysine production pathway,
4) a molecule involved in the tryptophan pathway,
5) a molecule involved in the threonine pathway,
6) a molecule involved in the acetyl-CoA production pathway, and/or
7) a molecule involved in the de-novo or salvage purine and pyrimidine pathways.

147. The Saccharopolyspora strain library of claim 146, wherein:
1) the molecule involved in the spinosyn synthesis pathway is a spinosyn;
2) the molecule involved in the SAM/methionine pathway is alpha-methyl methionine (aMM) or norleucine;
3) the molecule involved in the lysine production pathway is thialysine or a mixture of alpha-ketobytarate and aspartate hydoxymate;
4) the molecule involved in the tryptophan pathway is azaserine or 5-fuoroindole;
5) the molecule involved in the threonine pathway is beta-hydroxynorvaline;
6) the molecule involved in the acetyl-CoA production pathway is cerulenin; and
7) the molecule involved in the de-novo or salvage purine and pyrimidine pathways is a purine or a pyrimidine analog.

148. The Saccharopolyspora strain library of claim 147, wherein the molecule is spinosyn J/L, and wherein each strain is resistant to about 50 ug/ml to about 2 mg/ml spinosyn J/L.

149. The Saccharopolyspora strain library of claim 147, wherein the molecule is alpha-methyl methionine (aMM), wherein each strain is resistant to about 1 mM to about 5 mM aMM.

150. A Saccharopolyspora strain comprising a reporter gene, wherein the reporter gene is selected from the group consisting of:
a) genes encoding a green fluorescent reporter protein, optionally the genes are codon optimized for expression in Saccharopolyspora;
b) genes encoding a green fluorescent reporter protein, optionally the genes are codon optimized for expression in Saccharopolyspora; and
c) genes encoding a beta-glucuronidase (gusA) protein, optionally the genes are codon optimized for expression in Saccharopolyspora.

151. The Saccharopolyspora strain of claim 150, wherein:
a) the green fluorescent reporter protein has the amino acid sequence of SEQ ID No. 143;
b) the red fluorescent reporter protein has the amino acid sequence of SEQ ID No. 144; and
c) the gusA protein has the amino acid sequence of SEQ ID No. 145.

152. The Saccharopolyspora strain of claim 150, wherein:
a) the gene encoding the green fluorescent reporter protein has the sequence of SEQ ID No. 81;
b) the gene encoding the red fluorescent reporter protein has the sequence of SEQ ID No. 82; and
c) the gene encoding the gusA protein has sequence of SEQ ID No. 83.

153. The Saccharopolyspora strain of claim 150, wherein the strain comprises both the gene encoding the green fluorescent reporter protein, and the gene encoding the red fluorescent reporter protein, wherein the fluorescent excitation and emission spectra of the green fluorescent reporter protein and the red fluorescent reporter protein are distinct from each other.

154. The Saccharopolyspora strain of claim 150, wherein the strain comprises both the gene encoding the green fluorescent reporter protein, and the gene encoding the red fluorescent reporter protein, wherein the fluorescent excitation and emission spectra of the green fluorescent reporter protein and the red fluorescent reporter protein are distinct from the endogenous fluorescence of the Saccharopolyspora strain.

155. A Saccharopolyspora strain comprising a DNA fragment integrated into one or more neutral integration sites in the genome of the Saccharopolyspora strain, wherein the neutral integration sites are selected from the group of positions within a genomic fragment having a sequence selected from SEQ ID Nos. 132-142, or genomic fragments homologous to any one of SEQ ID Nos. 132-142.

156. The Saccharopolyspora strain of claim 155, wherein the Saccharopolyspora strain has a desired level of improved phenotypic performance compared to the phenotypic performance of a reference Saccharopolyspora strain without the integrated DNA fragment.

157. The Saccharopolyspora strain of claim 156, wherein the Saccharopolyspora strain has a desired level of improved spinosyn production compared to the phenotypic performance of a reference Saccharopolyspora strain without the integrated DNA fragment.

158. The Saccharopolyspora strain of claim 155, wherein the integrated DNA fragment comprises a sequence encoding for a reporter protein.

159. The Saccharopolyspora strain of claim 155, wherein the integrated DNA fragment comprises a transposon.

160. The Saccharopolyspora strain of claim 155, wherein the integrated DNA fragment comprises an attachment site (attB) which can be recognized by its corresponding integrase.

161. A method of integrating a DNA fragment into the genome of a Saccharopolyspora strain, wherein the DNA fragment is integrated into a neutral integration site in the genome of the Saccharopolyspora strain, wherein the neutral integration site is selected from the group of positions within a genomic fragment having a sequence selected from SEQ ID Nos. 132-142, or genomic fragments homologous to any one of SEQ ID Nos. 132-142.

162. The method of integrating a DNA fragment into the genome of a Saccharopolyspora strain of claim 161, wherein the DNA fragment comprises an attachment site (attB) which can be recognized by its corresponding integrase.

163. A method for rapidly consolidating genetic mutations derived from at least two parental Saccharopolyspora strains, comprising the steps of:
(1) providing at least two parental Saccharopolyspora strains, wherein each strain comprises a unique genomic mutation that does not exist in the other strains;
(2) preparing protoplasts from each of the parental strains;
(3) fusing the protoplasts from the parental strains to produce fused protoplast comprising the genomes of two parental Saccharopolyspora strains, wherein homologous recombination between the genomes of each parental strain occurs;
(4) recovering Saccharopolyspora cells from the fused protoplast produced in step (3); and
(5) selecting for Saccharopolyspora cells comprising the unique genomic mutation of a first parental Saccharopolyspora strain; and
(6) genotyping the Saccharopolyspora cells obtained in step (5) for the presence of the unique genomic mutation of a second parental strain,
thereby obtaining a new Saccharopolyspora strain comprising the unique genomic mutations derived from two parental Saccharopolyspora strains.

164. The method of claim 163, wherein one of the unique genomic mutations is linked to a selectable marker, while the other unique genomic mutation is not linked to any selectable marker.

165. The method of claim 164, wherein in step (3) the ratio of protoplasts of the stain originally containing the unique genomic mutation linked to the selectable marker:protoplasts of the stain originally containing the unique genomic mutation not linked to the selectable marker is less than 1:1.

166. The method of claim 165, wherein the ratio is about 1:10 to about 1:100, or less.

167. The method of claim 163, wherein in step (4), protoplast cells are plated on an osmotically stabilized media without the use of agar overlay.

168. The method of claim 163, wherein step (5) is accomplished by overlaying an appropriate selection drug antibiotic onto the growing cells, when one of the unique genomic mutations is linked to a selectable marker which results in resistance to the selection drug.

169. The method of claim 163, wherein step (5) is accomplished by genotyping, when none of the unique genomic mutations is linked to a selectable marker.

170. The method of claim 163, wherein genetic mutations derived from more than two strains are randomly consolidated during a single consolidation process.

171. The method of claim 163, wherein in step (2) the protoplasts are initially collected by centrifuging at a speed about 5000×g for about 5 minutes.

172. The method of claim 163, wherein the method does not comprise of filtrating the protoplasts through cotton wool.

173. The method of claim 163, wherein the fused protoplasts are recovered on a R2YE media rather than top-agar.

174. The method of claim 173, wherein the R2YE media comprises 0.5M sorbitol and 0.5M mannose.

175. A method of targeted genome editing in a Saccharopolyspora strain, comprising:
a) introducing a plasmid comprising a selection marker, a counterselection marker, a DNA fragment having homology to the genomic locus of the Saccharopolyspora strain to be edited, and plasmid backbone sequence into a base Saccharopolyspora strain;
b) selecting for Saccharopolyspora strains with integration event based on the presence of the selection marker in the genome;
c) selecting for Saccharopolyspora strains having the plasmid backbone looped out based on the absence of the counterselection marker gene, wherein the counterselection marker is a sacB gene or a pheS gene.

176. The method of claim 175, wherein the resulted Saccharopolyspora strain with edited genome has better performance compared to the parent strain without the editing.

177. The method of claim 176, wherein the resulted Saccharopolyspora strain has increased spinosyn production compared to the parent strain without the editing.

178. The method of claim 175, wherein the sacB gene is codon-optimized for Saccharopolyspora spinosa.

179. The method of claim 178, wherein the sacB gene encodes an amino acid sequence with 90% sequence identity to the amino acid sequence encoded by SEQ ID No. 146.

180. The method of claim 175, wherein the pheS gene is codon-optimized for Saccharopolyspora spinosa.

181. The method of claim 180, wherein the pheS gene encodes an amino acid sequence with 90% sequence identity to the amino acid encoded by SEQ ID No. 147 or SEQ ID No. 148.

182. A method of transferring genetic material from donor microorganism cells to recipient cells of a Saccharopolyspora microorganism, wherein the method comprises the steps of:
1) Optionally, subculturing recipient cells to late-exponential or stationary phase;
2) Optionally, subculturing donor cells to mid-exponential phase;
3) Combining donor and recipient cells;
4) Plating donor and recipient cell mixture on conjugation media;
5) Incubating plates to allow cells to conjugate;
6) Applying antibiotic selection against donor cells;
7) Applying antibiotic selection against non-integrated recipient cells; and
8) further incubating plates to allow for the outgrowth of integrated recipient cells.

183. The method of claim 182, wherein the donor microorganism cells are E. coli cells.

184. The method of claim 182, wherein at least two, three, four, five, six, seven or more of the following conditions are utilized:
1) recipient cells are washed before conjugating;
2) donor cells and recipient cells are conjugated at a temperature of about 30° C.;
3) recipient cells are sub-cultured for at least about 48 hours before conjugating;
4) the ratio of donor cells:recipient cells for conjugation is about 1:0.6 to 1:1.0;
5) an antibiotic drug for selection against the donor cells is delivered to the mixture about 15 to 24 hours after the donor cells and the recipient cells are mixed;
6) an antibiotic drug for selection against the recipient cells is delivered to the mixture about 40 to 48 hours after the donor cells and the recipient cells are mixed;
7) the conjugation media plated with donor and recipient cell mixture is dried for at least about 3 hours to 10 hours;
8) the conjugation media comprises at least about 3 g/L glucose;
9) the concentration of donor cells is about OD600=0.1 to 0.6;
10) the concentration of recipient cells is about OD540=5.0 to 15.0;

185. The method of claim 184, wherein the antibiotic drug for selection against the donor cells is nalidixic, and the concentration is about 50 to about 150 μg/ml.

186. The method of claim 185, wherein the antibiotic drug for selection against the donor cells is nalidixic, and the concentration is about 100 μg/ml.

187. The method of claim 184, wherein the antibiotic drug for selection against the recipient cells is apramycin, and the concentration is about 50 to about 250 μg/ml.

188. The method of claim 187, wherein the antibiotic drug for selection against the recipient cells is apramycin, and the concentration is about 100 μg/ml.

189. The method of claim 182, wherein the method is performed in a high-throughput process.

190. The method of claim 189, wherein the method is performed on a 48-well Q-trays.

191. The method of claim 189, wherein the high-throughput process is automated.

192. The method of claim 191, where the mixture of donor cells and recipient cells is a liquid mixture, and ample volume of the liquid mixture is plated on the medium with a rocking motion, wherein the liquid mixture is dispersed over the whole area of the medium.

193. The method of claim 191, wherein the method comprises automated process of transferring exconjugants by colony picking with yeast pins for subsequent inoculation of recipient cells with integrated DNA provided by the donor cells.

194. The method of claim 193, the colony picking is performed in either a dipping motion, or a stirring motion.

195. The method of claim 184, wherein the conjugating media is a modified ISP4 media comprising about 3-10 g/L glucose.

196. The method of claim 184, wherein the total number of donor cells or recipient cells in the mixture is about 5×10⁶ to about 9×10⁶.

197. The method of claim 182, wherein the method is performed with at least four of the following conditions:
1) recipient cells are washed before conjugating;
2) donor cells and recipient cells are conjugated at a temperature of about 30° C.;
3) recipient cells are sub-cultured for at least about 48 hours before conjugating;
4) the ratio of donor cells:recipient cells for conjugation is about 1:0.8;
5) an antibiotic drug for selection against the donor cells is delivered to the mixture about 20 hours after the donor cells and the recipient cells are mixed;
6) the amount of the donor cells or the amount of the recipient cells in the mixture is about 7×10⁶, and
7) the conjugation media comprises about 6 g/L glucose.

198. A method of targeted genomic editing in a Saccharopolyspora strain, resulting in a scarless Saccharopolyspora strain containing a genetic variation at a targeted genomic locus, comprising:
a) introducing a plasmid into a Saccharopolyspora strain, said plasmid comprising:
i. a selection marker,
ii. a counterselection marker,
iii. a DNA fragment containing a genetic variation to be integrated into the Saccharopolyspora genome at a target locus, said DNA fragment having homology arms to the target genomic locus flanking the desired genetic variation, and
iv. plasmid backbone sequence;
b) selecting for a Saccharopolyspora strain that has undergone an initial homologous recombination and has the genetic variation integrated into the target locus based on the presence of the selection marker in the genome; and
c) selecting for a Saccharopolyspora strain that has the genetic variation integrated into the target locus, but has undergone an additional homologous recombination that loops-out the plasmid backbone, based on the absence of the counterselection marker,
wherein said targeted genomic locus may comprise any region of the Saccharopolyspora genome, including genomic regions that do not contain repeating segments of encoding DNA modules.

199. The method of claim 198, wherein the plasmid does not comprise a temperature sensitive replicon.

200. The method of claim 198, wherein the plasmid does not comprise an origin of replication.

201. The method of claim 198, wherein the selection step (c) is performed without replication of the integrated plasmid.

202. The method of claim 198, wherein the plasmid is a single homologous recombination vector.

203. The method of claim 198, wherein the plasmid is a double homologous recombination vector.

204. The method of claim 198, wherein the counterselection marker is a sacB gene or a pheS gene.

205. The method of claim 204, wherein the sacB gene or pheS gene is codon-optimized for Saccharopolyspora spinosa.

206. The method of claim 205, wherein the sacB gene encodes an amino acid sequence with 90% identity to the amino acid sequence encoded by SEQ ID NO. 146.

207. The method of claim 205, wherein the pheS gene encodes an amino acid sequence with 90% sequence identity to the amino acid encoded by SEQ ID NO. 147 or SEQ ID NO. 148.

208. The method of claim 198, wherein the plasmid is introduced into the Saccharopolyspora strain by transformation.

209. The method of claim 198, wherein the transformation is a protoplast transformation.

210. The method of claim 198, wherein the plasmid is introduced into the Saccharopolyspora strain by conjugation, wherein the Saccharopolyspora strain is a recipient cell, and a donor cell comprising the plasmid transfers the plasmid to the Saccharopolyspora strain.

211. The method of claim 198, wherein the conjugation is based on an E. coli donor cell comprising the plasmid.

212. The method of claim 198, wherein the target locus is a locus associated with production of a compound of interest in the Saccharopolyspora strain.

213. The method of claim 198, wherein the resulting Saccharopolyspora strain has increased production of a compound of interest compared to a control strain without the genomic editing.

214. The method of claim 212 or claim 213, wherein the compound of interest is a spinosyn.

215. The method of claim 198, wherein the method is performed as a high-throughput procedure.
